(19) United States
(12) Patent Application Publication
(10) Pub. No.: US 2021/0024971 A1
(43) Pub. Date: Jan. 28, 2021

(54) METHODS FOR PRODUCING, DISCOVERING, AND OPTIMIZING LASSO PEPTIDES

(71) Applicant: Lassogen, Inc., San Diego, CA (US)

(72) Inventors: Mark J. Burk, San Diego, CA (US); I-Hsiung Brandon Chen, San Diego, CA (US)

(21) Appl. No.: 17/043,605

(22) PCT Filed: Mar. 29, 2019
(86) PCT No.: PCT/US2019/024811

§ 371 Date: Sep. 29, 2020

Related U.S. Application Data
(60) Provisional application No. 62/652,213, filed on Apr. 3, 2018.
(60) Provisional application No. 62/651,028, filed on Mar. 30, 2018.

(51) Int. Cl. C12P 21/02; C07K 14/47; G01N 33/68; C12N 15/10; C12N 9/52

(52) U.S. Cl. CPC C12P 21/02; C07K 14/4723; G01N 2500/10; C12N 15/1089; C12N 9/52; G01N 33/6845

(57) ABSTRACT

Provided herein are lasso peptides and methods and systems of synthesizing lasso peptides, methods of discovering lasso peptides, methods of optimizing the properties of lasso peptides, and methods of using lasso peptides.

This application claims the benefit of priority to U.S. Provisional Patent Application No. 62/651,028 filed Mar. 30, 2018 and U.S. Provisional Patent Application No. 62/652,213 filed Apr. 3, 2018, the disclosure of each of which is incorporated by reference herein in its entirety.

The instant application contains a Sequence Listing which has been submitted electronically in ASCII format and is hereby incorporated by reference in its entirety. Said ASCII copy, created on Mar. 28, 2019, is named 12956-445-228_SL.txt and is 1,681,979 bytes in size.

### 1. FIELD

The field of invention covers methods for synthesis, discovery, and optimization of lasso peptides, and uses thereof.

### 2. BACKGROUND

Peptides serve as useful tools and leads for drug development since they often combine high affinity and specificity for their target receptor with low toxicity. In addition, peptides are potentially much safer drugs since degradation in the body affords non-toxic, nutritious amino acids. (Sato, A K., et al., Curr. Opin. Biotechnol, 2006, 17, 638-642; Antosova, Z., et al., Trends Biotechnol., 2009, 27, 628-635). However, their clinical use as efficacious drugs has been limited due to undesirable physicochemical and pharmacokinetic properties, including poor solubility and cell permeability, low bioavailability, and instability due to rapid proteolytic degradation under physiological conditions (Antosova, Z., et al., Trends Biotechnol, 2009, 27, 628-635).

Peptides with a knotted topology may be used as stable molecular frameworks for potential therapeutic applications. For example, ribosomally assembled natural peptides sharing the cyclic cysteine knot (CCK) motif, have been recently characterized (Weidmann, J.; Craik, D. J., J. Experimental Bot., 2016, 67, 4801-4812; Burman, R, et al., J. Nat. Prod. 2014, 77, 724-736; Reinwarth, M., et al., Molecules, 2012, 17, 12533-12552; Lewis, R J., et al., Pharmacol. Rev., 2012, 64, 259-298). These knotted peptides require the formation of three disulfide bonds to hold them into a defined conformation. However, these knotted peptide scaffolds are not readily accessible by genetic manipulation and heterologous production in cells and discovery relies on traditional extraction and fractionation methods that are slow and costly. Moreover, their production relies either on solid phase peptide synthesis (SPPS) or on expressed protein ligation (EPL) methods to generate the circular peptide backbone, followed by oxidative folding to form the correct three disulfide bonds required for the knotted structure (Craik, D. J., et al., Cell Mol. Lift Sci. 2010, 67, 9-16; Benade, L. & Camarero, J. A. Cell Mol. Lift Sci., 2009, 66, 3909-22).

Thus, there exists a need for new classes of peptide-based therapeutic compounds with readily available methods for their discovery, genetic manipulation and optimization, cost-effective production, and high-throughput screening. The inventions described herein meet these needs in the field.

### 3. SUMMARY

Provided herein are lasso peptides and methods and systems of synthesizing lasso peptides, methods of discovering lasso peptides, methods of optimizing the properties of lasso peptides, and methods of using lasso peptides.

In some embodiments, provided herein are methods for production and optional screening of one or more lasso peptides (LPs) or one or more lasso peptide analogs or their combination using a cell-free biosynthesis (CFB) reaction mixture, comprising the steps: (i) combining and contacting one or more lasso precursor peptides (LPP), one or more lasso core peptide (LCP), or their combination, with a lasso cyclase (LCase) enzyme, and optionally with a lasso peptidase (LPase) enzyme when the one or more LPP is present, in a CFB reaction mixture; (ii) synthesizing the one or more lasso peptides or LP analogs in the CFB reaction mixture, and (iii) optionally screening the one or more lasso peptides or LP analogs for one or more desired properties or activities by (1) screening the CFB reaction mixture, or (2) screening the partially purified or substantially purified lasso peptide or LP analog.

In some embodiments, the method further comprises: (i) obtaining at least one of the LPP, the LCP, the LPase or the LCase by chemical synthesis or by biological synthesis, optionally; (ii) where the biological synthesis comprises transcription and/or translation of a gene or oligonucleotide encoding the LCP, a gene or oligonucleotide encoding the LPP, a gene or oligonucleotide encoding the LPAse, or a gene or oligonucleotide encoding the LCase, and optionally where the transcription and/or translation of these genes or oligonucleotides occurs in the CFB reaction mixture.

In some embodiments, the method further comprising: (i) designing the LP gene or oligonucleotide, the LPP gene or oligonucleotide, the LPase gene or oligonucleotide, or the LCase gene or oligonucleotide for transcription and/or translation in the CFB reaction mixture, and optionally; where the designing uses genetic sequences for the lasso precursor peptide gene, the lasso core peptide gene, the lasso peptidase gene, and/or the lasso cyclase gene, and optionally where the genetic sequences are identified using a genome-mining algorithm, and optionally where the genome-mining algorithm is anti-SMASH, BAGEL3, or RODEO.

In some embodiments, in any of the preceding methods, wherein the combining and contacting comprises a minimal set of lasso peptide biosynthesis components in the CFB reaction mixture, where the minimal set of lasso peptide biosynthesis components comprises the one or more lasso precursor peptides (A), one lasso peptidase (B), and one lasso cyclase (C), each of which may be independently generated by the biological and/or chemical synthesis methods, or the minimal set optionally further comprises the one or more lasso core peptide and one lasso cyclase, each of which may be independently generated by the biological and/or the chemical synthesis methods.

In some embodiments, in any preceding methods, wherein the CFB reaction mixture contains a minimal set of lasso peptide biosynthesis components and comprises one or more of: (i) a substantially isolated lasso precursor peptide or lasso precursor peptide fusion, a substantially isolated lasso cyclase enzyme or fusion thereof, and a substantially isolated lasso peptidase enzyme or fusion thereof, or (ii) oligonucleotides (linear or circular constructs of DNA or RNA) that encode for a lasso precursor peptide or a fusion thereof, a substantially isolated lasso cyclase enzyme or fusion thereof, and a substantially isolated lasso peptidase enzyme or fusion thereof, or (iii) a substantially isolated precursor peptide or fusion thereof, an oligonucleotide that encodes for a lasso cyclase or fusion thereof, and an oligonucleotide that encodes for a lasso peptidase or fusion thereof, or (iv) an oligonucleotide that encodes for a precursor peptide, an oligonucleotide that encodes for a lasso cyclase or fusion thereof, and an oligonucleotide that encodes for a lasso peptidase, or fusion thereof, or (v) a substantially isolated lasso core peptide or fusion thereof and a substantially isolated lasso cyclase or fusion thereof, or (vi) an oligonucleotide that encodes for a lasso core peptide and a substantially isolated lasso cyclase or fusion thereof, or (vii) an oligonucleotide that encodes for a lasso core peptide and an oligonucleotide that encodes for a lasso cyclase or fusion thereof.

In some embodiments, in any preceding methods, the lasso precursor (A) is a peptide or polypeptide produced chemically or biologically, with a sequence corresponding to the even number of SEQ ID Nos: 1-2630 or a sequence with at least 30% identity of the even number of SEQ ID Nos: 1-2630, or a protein or peptide fusion or portion thereof. In any preceding methods, wherein the lasso peptidase (B) is an enzyme produced chemically or biologically, with a sequence corresponding to peptide Nos 1316-2336 or a natural sequence with at least 30% identity of peptide Nos: 1316-2336.

In some embodiments, in any preceding methods, wherein the lasso cyclase (C) is an enzyme produced chemically or biologically with a sequence corresponding to peptide Nos: 2337-3761 or a natural sequence with at least 30% identity of peptide Nos: 2337-3761.

In some embodiments, in any preceding methods, wherein the CFB reaction mixture further comprises one or more RiPP recognition elements (RREs) or the genes encoding such RREs. In some embodiments, in any preceding methods, wherein the RiPP recognition elements (RREs) are proteins produced chemically or biologically with a natural sequence corresponding to peptide Nos: 3762-4593 or a natural sequence of at least 30% identity of peptide Nos: 3762-4593.

In some embodiments, in any preceding methods, wherein the CFB reaction mixture contains a lasso peptidase or a lasso cyclase that is fused at the N- or C-terminus with one or more RiPP recognition elements (RREs).

In some embodiments, in any preceding methods, wherein the one or more lasso peptide or the one or more lasso peptide analog or their combination is produced.

In some embodiments, in any preceding methods, wherein the one or more lasso peptides or the one or more lasso peptide analogs or their combination is produced and screened.

In some embodiments, in any preceding methods, wherein the one or more lasso core peptide or lasso peptide or lasso peptide analogs, containing no fusion partners, comprises at least eleven amino acid residues and a maximum of about fifty amino acid residues.

In some embodiments, in any preceding methods, wherein the CFB reaction mixture (or system) comprises a whole cell extract, a cytoplasmic extract, a nuclear extract, or any combination thereof, wherein each are independently derived from a prokaryotic or a eukaryotic cell.

In some embodiments, in any preceding methods, wherein the CFB reaction mixture comprises substantially isolated individual transcription and/or translation components derived from a prokaryotic or a eukaryotic cell.

In some embodiments, in any preceding methods, wherein the CFB reaction mixture further comprises one or more lasso peptide modifying enzymes or genes that encode the lasso peptide modifying enzymes, and optionally wherein the one or more lasso peptide modifying enzymes is independently selected from the group consisting of N-methyltransferases, O-methyltransferases, biotin ligases, glycosyltransferases, esterases, acylases, acyltransferases, aminotransferases, amidases, hydroxylases, dehydrogenases, halogenases, kinases, RiPP heterocyclases, RiPP cyclodehydratases, and prenyltransferases.

In some embodiments, in any preceding methods, wherein the CFB reaction mixture comprises a buffered solution comprising salts, trace metals, ATP and co-factors required for activity of one or more of the LPase, the LCase, an enzyme required for the translation, an enzyme required for the transcription, or a lasso peptide modifying enzyme.

In some embodiments, in any preceding methods, wherein the CFB reaction mixture comprises the substantially isolated lasso precursor peptides or lasso core peptide, or fusions thereof, combined and contacted with the substantially isolated enzymes that include a lasso cyclase, and optionally a lasso peptidase, or fusions thereof, in a buffered solution containing salts, trace metals, ATP, and co-factors required for enzymatic activity

In some embodiments, in any preceding methods, wherein the CFB system is used to facilitate the discovery of new lasso peptides from Nature, further comprising the steps: (i) analyzing bacterial genome sequence data and predict the sequence of lasso peptide gene clusters and associated genes, optionally using the genome-mining algorithm, optionally where the genome-mining algorithm is anti-SMASH, BAGEL3, or RODEO, (ii) cloning or synthesizing the minimal set of lasso peptide biosynthesis genes (A-C) or oligonucleotides containing these gene sequences, and (iii) synthesizing known or previously undiscovered natural lasso peptides using the cell-free biosynthesis methods described herein.

In some embodiments, in any preceding methods, wherein the one or more lasso peptides, the one or more lasso peptide analogs, or their combination comprises a library containing at least one lasso peptide analog in which at least one amino acid residue is changed from its natural residue.

In some embodiments, in any preceding methods, wherein the one or more lasso peptides, the one or more lasso peptide analogs, or their combination comprises a library wherein substantially all or all amino acid mutational variants of the lasso core peptide or the lasso precursor peptide, optionally where the amino acid mutational variants of the lasso core peptide or the lasso precursor peptide are obtained by biological or chemical synthesis, and optionally where the biological synthesis uses a gene library encoding substantially all or all genetic mutational variants of the lasso core peptide or the lasso precursor peptide, optionally where the gene library is rationally designed, and optionally where the mutational variants of the lasso core peptide or the lasso precursor peptide are converted to lasso peptide mutational variants, and optionally where the lasso peptide mutational variants are screened for desired properties or activities.

In some embodiments, a library of lasso peptides or lasso peptide analogs is created by (1) directed evolution technologies, or (2) chemical synthesis of lasso precursor peptide or lasso core peptide variants and enzymatic conversion to lasso peptide mutational variants, or (3) display technologies, optionally wherein the display technologies are in vitro display technologies, and optionally wherein in vitro display technologies are RNA or DNA display technologies, or combination thereof, and optionally where the library of lasso peptides or lasso peptide analogs is screened for desired properties or activities.

In some embodiments, provided herein is a lasso peptide library, a LP analog library or a combination thereof, comprising at least two lasso peptides, at least two lasso peptide analogs, or at least one lasso peptide and one lasso peptide analog, which may be pooled together in one vessel or where each member is separated into individual vessels (e.g., wells of a plate), and wherein the library members are isolated and purified, or partially isolated and purified, or substantially isolated and purified, or optionally wherein the library members are contained in a CFB reaction mixture.

In some embodiments, the library is created using the system and methods provided herein.

In some embodiments, the CFB reaction mixture useful for the synthesis of lasso peptides and lasso peptide analogs comprising one or more cell extracts or cell-free reaction media that support and facilitate a biosynthetic process wherein one or more lasso peptides or lasso peptide analogs is formed by converting one or more lasso precursor peptides or one or more lasso core peptides through the action of a lasso cyclase, and optionally a lasso peptidase, and optionally wherein transcription and/or translation of oligonucleotide inputs occurs to produce the lasso cyclase, lasso peptidase, lasso precursor peptides, and/or lasso core peptides.

In some embodiments, the CFB reaction mixture further comprising a supplemented cell extract.

In some embodiments, the CFB reaction mixture also comprises the oligonucleotides, genes, biosynthetic gene clusters, enzymes, proteins, and final peptide products, including lasso precursor peptides, lasso core peptides, lasso peptides, or lasso peptide analogs that result from performing a CFB reaction.

In some embodiments, provided herein are a kit for the production of lasso peptides and/or lasso peptide analogs according to any of the preceding methods comprising a CFB reaction mixture, a cell extract or cell extracts, cell extract supplements, a lasso precursor peptide or gene or a library of such, a lasso core peptide or gene or a library of such, a lasso cyclase or gene or genes, and/or a lasso peptidase or gene, along with information about the contents and instructions for producing lasso peptides or lasso peptide analogs.

In some embodiments, provided herein is a lasso peptidase library comprising at least two lasso peptidases, wherein the lasso peptidases are encoded by genes of a same organism or encoded by genes of different organisms. In some embodiments, each lasso peptidase of the at least two lasso peptidases comprises an amino acid sequence selected from peptide Nos: 1316-2336, or a natural sequence with at least 30% identity of peptide Nos: 1316-2336. In some embodiments, the library is produced by a cell-flee biosynthesis system.

In some embodiments, provided herein is a lasso cyclase library comprising at least two lasso cyclases, wherein the lasso cyclases are encoded by genes of a same organism or encoded by genes of different organisms. In some embodiments, each lasso peptidase of the at least two lasso cyclases comprises an amino acid sequence selected from peptide Nos: 2337-3761, or a natural sequence having at least 30% identity of peptide Nos: 2337-3761. In some embodiments, the natural sequence is identified using a genome mining tool as described herein. In some embodiments, the lasso cyclase library is produced by a cell-flee biosynthesis system.

In some embodiments, provided herein is a cell flee biosynthesis (CFB) system for producing one or more lasso peptide or lasso peptide analogs, wherein the CFB system comprises at least one component capable of producing one or more lasso precursor peptide. In some embodiments, the CFB system further comprises at least one component capable of producing one or more lasso peptidase. In some embodiments, the CFB system further comprises at least one component capable of producing one or more lasso cyclase. In some embodiments, the at least one component capable of producing the one or more lasso precursor peptide comprises the one or more lasso precursor peptide. In some embodiments, the one or more lasso precursor peptide is synthesized outside the CFB system.

In some embodiments, the one or more lasso precursor peptide is isolated from a naturally-occurring microorganism.

In some embodiments, the one or more lasso precursor peptide is isolated from a plurality naturally-occurring microorganisms.

In some embodiments, the lasso precursor peptide is isolated as a cell extract of the naturally occurring microorganism.

In some embodiments, the at least one component capable of producing the one or more lasso precursor peptide comprises a polynucleotide encoding for the one or more lasso precursor peptide. In some embodiments, the polynucleotide comprises a genomic sequence of a naturally-existing microbial organism. In some embodiments, the polynucleotide comprises a mutated genomic sequence of a naturally-existing microbial organism. In some embodiments, the polynucleotide comprises a plurality polynucleotides. In some embodiments, the plurality of polynucleotides each comprises a genomic sequence of a naturally existing microbial organism and/or a mutated genomic sequence of a naturally existing microbial organism. In some embodiments, the at least two of the plurality of polynucleotides comprise genomic sequences or mutated genomic sequences of different naturally existing microbial organisms. In some embodiments, the polynucleotide comprises a sequence selected from the odd numbers of SEQ ID Nos: 1-2630, or a homologous sequence having at least 30% identity of the odd numbers of SEQ ID Nos: 1-2630.

In some embodiments, the at least one component capable of producing the one or more lasso peptidase comprises the one or more lasso peptidase. In some embodiments, the one or more lasso peptidase is synthesized outside the CFB system. In some embodiments, the one or more lasso peptidase is isolated from a naturally-occurring microorganism. In some embodiments, the lasso peptidase is isolated as a cell extract of the naturally occurring microorganism.

In some embodiments, the at least one component capable of producing the one or more lasso peptidase comprises a polynucleotide encoding for the one or more lasso peptidase.

In some embodiments, the polynucleotide encoding for the lasso peptidase comprises a genomic sequence of a naturally-existing microbial organism. In some embodiments, the polynucleotide encoding for the one or more lasso peptidase comprises a plurality of polynucleotide encoding for the one or more lasso peptidase. In some embodiments, the plurality of polynucleotides each comprises a genomic sequence of a naturally existing microbial organism. In some embodiments, the at least two of the plurality of polynucleotides encoding the one or more lasso peptidase comprise genomic sequences of different naturally existing microbial organisms.

In some embodiments, the at least one component capable of producing the one or more lasso cyclase comprises the one or more lasso cyclase. In some embodiments, the one or more lasso cyclase is synthesized outside the CFB system. In some embodiments, the one or more lasso cyclase is isolated from a naturally-occurring microorganism. In some embodiments, the at least two of the one or more lasso cyclases are isolated from different naturally-occurring microorganisms. In some embodiments, the lasso peptidase is isolated as a cell extract of the naturally occurring microorganism.

In some embodiments, the at least one component capable of producing the one or more lasso cyclase comprises a polynucleotide encoding for the one or more lasso cyclase. In some embodiments, the at least one component capable of producing the one or more lasso cyclase comprises a plurality of polynucleotides encoding for the one or more lasso cyclase. In some embodiments, the polynucleotide encoding for the lasso cyclase comprises a genomic sequence of a naturally-existing microbial organism. In some embodiments, the at least two of the plurality of polynucleotides encoding the one or more lasso cyclase comprise genomic sequences of different naturally existing microbial organisms.

In some embodiments, the one or more lasso precursor peptide each comprises an amino acid sequence selected from the even number of SEQ ID Nos: 1-2630 or a sequence having at least 30% identity to the even number of SEQ ID Nos: 1-2630. In some embodiments, the one or more lasso peptidase each comprises an amino acid sequence selected from peptide Nos: 1316-2336 or a natural sequence having at least 30% identity to peptide Nos: 1316-2336. In some embodiments, the one or more lasso peptidase each comprises an amino acid sequence selected from peptide Nos: 2337-3761 or a natural sequence having at least 30% identity of peptide Nos: 2337-3761. In some embodiments, wherein the natural sequence is identified using a genomic mining tool described herein. In some embodiments, the CFB system further comprises at least one component capable of producing one or more RIPP recognition element (RRE).

In some embodiments, the one or more RRE each comprises an amino acid sequence selected from peptide Nos: 3762-4593, or a natural sequence having at least 30% identity of peptide Nos: 3762-4593. In some embodiments, the at least one component capable of producing the one or more RRE comprises the one more RRE. In some embodiments, the RRE comprises at least one component capable of producing the one or more RRE comprises a polynucleotide encoding for the one or more RRE. In some embodiments, the polynucleotide encoding for the one or more RRE comprises a plurality of polynucleotides encoding for the one or more RRE. In some embodiments, the polynucleotide encoding for the one or more RRE comprises a genomic sequence or a naturally existing microorganism. In some embodiments, at least two of the plurality of polynucleotides encoding the one or more RREs comprise genomic sequences of different naturally existing microbial organisms.

In some embodiments, the CFB system comprises a minimal set of lasso biosynthesis components. In some embodiments, the CFB system is capable of producing a combination of (i) lasso precursor peptide or a lasso core peptide, (ii) lasso cyclase, and (iii) lasso peptidase as listed in Table 1. In some embodiments, the CFB system is capable of producing a lasso peptide library. In some embodiments, the CFB system comprises a cell extract. In some embodiments, the CFB system comprises a supplemented cell extract. In some embodiments, the CFB system comprises a CFB reaction mixture. In some embodiments, the CFB system is capable of producing at least one lasso peptide or lasso peptide analog when incubated under a suitable condition. In some embodiments, the suitable condition is a substantially anaerobic condition. In some embodiments, the CFB comprises a cell extract, and the suitable condition comprises the natural growth condition of the cell where the cell extract is derived.

In some embodiments, the CFB system is in the form of a kit. In some embodiments, the one or more components of the CFB systems are separated into a plurality of parts forming the kit. In some embodiments, the plurality of parts forming the kit, when separated from one another, are substantially free of chemical or biochemical activity.

### 4. BRIEF DESCRIPTION OF THE FIGURES

The details of one or more embodiments of the invention are set forth in the accompanying drawings and the description below. Other features, objects, and benefits of the invention will be apparent from the description and drawings, and from the claims. All publications, patents and patent applications cited herein are hereby expressly incorporated by reference for all purposes.

The embodiments of the description described herein are not intended to be exhaustive or to limit the disclosure to the precise forms disclosed in the following drawings or detailed description. Rather, the embodiments are chosen and described so that others skilled in the art can appreciate and understand the principles and practices of the description.

FIG. 1A is a schematic illustration of the conversion of a lasso precursor peptide into a lasso peptide 1 with the lasso (lariat) topology.

FIG. 1B is a schematic illustration of the conversion of a lasso precursor peptide into a lasso peptide, where the leader peptidase (enzyme B) cleaves the leader sequence and conformationally positions the linear core peptide for closure, and the lasso cyclase (enzyme C) activates Glu or Asp at position 7, 8, or 9 of the core peptide and catalyzes cyclization with the N-terminus.

FIG. 2 shows a generalized 26-mer linear core peptide corresponding to a lasso peptide.

FIG. 3 is a schematic illustration of the process of discovering lasso peptide encoding genes by genomic mining, and cell-free biosynthesis of lasso peptide.

FIG. 4 is a schematic illustration of cell-flee biosynthesis of lasso peptides using in vitro transcription/translation, and construction of a lasso peptide library for screening of activities.

FIG. 5 illustrates a comparison between cell-based and cell-flee biosynthesis of lasso peptides.

FIG. 6 shows the results for detecting MccJ25 by LC/MS analysis.

FIG. 7 shows the results for detecting ukn22 by LC/MS analysis.

FIG. 8 shows the results for detecting capistruin, ukn22 and burhizin in individual vessels by MALDI-TOF analysis

FIG. 9 shows the results for detecting capistruin, ukn22 and burhizin in a single vessel by MALDI-TOF analysis

FIG. 10 shows the results for detecting ukn22 and five ukn22 variants, ukn22 W1Y, ukn22 W1F, ukn22 W1H, ukn22 W1L and ukn22 W1A, in individual vessels by MALDI-TOF analysis

FIG. 11 shows the results for detecting ukn22 and five ukn22 variants, ukn22 W1Y, ukn22 W1F, ukn22 W1H, ukn22 W1L and ukn22 W1A, in a single vessel by MALDI-TOF analysis.

FIG. 12 shows the results for detecting cellulonodin in a single vessel by MALDI-TOF analysis.

### 5. DETAILED DESCRIPTION

The novel features of this invention are set forth specifically in the appended claims. A better understanding of the features and benefits of the present invention will be obtained by reference to the following detailed description that sets forth illustrative embodiments, in which the principles of the invention are utilized. To facilitate a full understanding of the disclosure set forth herein, a number of terms are defined below.

### 5.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (4th ed. 2012); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2nd ed. 2010). Molecular Biology of the Cell (6th Ed., 2014). Organic Chemistry, (Thomas Sorrell, 1999). March's Advanced Organic Chemistry (6^{th} ed. 2007). Lasso Peptides, (Li, Y.; Zirah, S.; Rebuffet, S., Springer; New York, 2015).

### 5.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

As used herein, the singular terms “a,” “an,” and “the” include the plural reference unless the context clearly indicates otherwise.

Unless otherwise indicated, the terms “oligonucleotides” and “nucleic acids” are used interchangeably and are written left to right in 5′ to 3′ orientation; amino acid sequences are written left to right in amino to carboxy orientation, respectively. Therefore, in general, the codon at the 5′-terminus of an oligonucleotide will correspond to the N-terminal amino acid residue that is incorporated into a translated protein or peptide product. Similarly, in general, the codon at the 3′-terminus of an oligonucleotide will correspond to the C-terminal amino acid residue that is incorporated into a translated protein or peptide product. It is to be understood that this invention is not limited to the particular methodology, protocols, and reagents described, as these may vary, depending upon the context they are used by those of skill in the art.

As used herein, the term “naturally occurring” or “natural” or “native” when used in connection with naturally occurring biological materials such as nucleic acid molecules, oligonucleotides, amino acids, polypeptides, peptides, metabolites, small molecule natural products, host cells, and the like, refers to materials that are found in or isolated directly from Nature and are not changed or manipulated by humans. The term “natural” or “naturally occurring” refers to organisms, cells, genes, biosynthetic gene clusters, enzymes, proteins, oligonucleotides, and the like that are found in Nature and are unchanged relative to these components found in Nature. The term “wild-type” refers to organisms, cells, genes, biosynthetic gene clusters, enzymes, proteins, oligonucleotides, and the like that are found in Nature and are unchanged relative to these components found in Nature (in the wild).

As defined herein, the term “natural product” refers to any product, a small molecule, organic compound, or peptide produced by living organisms, e.g., prokaryotes or eukaryotes, found in Nature, and which are produced through natural biosynthetic processes. As defined herein, “natural products” are produced through an organism's secondary metabolism or through biosynthetic pathways that are not essential for survival and not directly involved in cell growth and proliferation.

As used herein, the term “non-naturally occurring” or “non-natural” or “unnatural” or “non-native” refer to a material, substance, molecule, cell, enzyme, protein or peptide that is not known to exist or is not found in Nature or that has been structurally modified and/or synthesized by humans. The term “non-natural” or “unnatural” or “non-naturally occurring” when used in reference to a microbial organism or microorganism or cell extract or gene or biosynthetic gene cluster of the invention is intended to mean that the microbial organism or derived cell extract or gene or biosynthetic gene cluster has at least one genetic alteration not normally found in a naturally occurring strain or a naturally occurring gene or biosynthetic gene cluster of the referenced species, including wild-type strains of the referenced species. Genetic alterations include, for example, introduction of expressible oligonucleotides or nucleic acids encoding polypeptides, other nucleic acid additions, nucleic acid deletions and/or other functional disruption of the microbial organism's genetic material. Such modifications include, for example, nucleotide changes, additions, or deletions in the genomic coding regions and functional fragments thereof, used for heterologous, homologous or both heterologous and homologous expression of polypeptides. Additional modifications include, for example, nucleotide changes, additions, or deletions in the genomic non-coding and/or regulatory regions in which the modifications alter expression of a gene or operon. Exemplary polypeptides include enzymes, proteins, or peptides within a lasso peptide biosynthetic pathway.

The terms “cell-free biosynthesis” and “CFB” are used interchangeably herein and refer to an in vitro (outside the cell) biosynthetic process that employs a “cell-flee biosynthesis reaction mixture”, including all the genes, enzymes, proteins, pathways, and other biosynthetic machinery necessary to carry out the biosynthesis of products, including RNA, proteins, enzymes, co-factors, natural products, small molecules, organic molecules, lasso peptides and the like, without the agency of a living cellular system.

The terms “cell-free biosynthesis system” and “CFB system” are used interchangeably and refer to the experimental design, set-up, apparatus, equipment, and materials, including a cell-flee biosynthesis reaction mixture and cell extracts, as defined below, that carries out a cell-free biosynthesis reaction and produce a desired product, such as a lasso peptide or lasso peptide analog.

The terms “cell-free biosynthesis reaction mixture” and “CFB reaction mixture” are used interchangeably and refer to the composition, in part or in its entirety, that enables a cell-flee biosynthesis reaction to occur and produce the biosynthetic proteins, enzymes, and peptides, as well as other products of interest, including but not limited to lasso precursor peptides, lasso core peptides, lasso peptides, or lasso peptide analogs. As defined herein, a “CFB reaction mixture” comprises one or more cell extracts or cell-free reaction media or supplemented cell extracts that support and facilitate a biosynthetic process in the absence of cells, wherein the CFB reaction mixture supports and facilitates the formation of a lasso peptide or lasso peptide analog through the activity of a lasso cyclase, and optionally the activity of a lasso peptidase, and optionally activities of polynucleotides that are converted into a lasso cyclase, a lasso peptidase, a lasso precursor peptide, a lasso core peptide, a lasso peptide, and/or a lasso peptide analog. A CFB reaction mixture may also comprise the oligonucleotides, genes, biosynthetic gene clusters, enzymes, proteins, and final peptide products, including lasso precursor peptides, lasso core peptides, lasso peptides, and/or lasso peptide analogs that result from performing a CFB reaction.

The terms “cell extract” and “cell-free extract” are used interchangeably and refer to the material and composition obtained by: (i) growing cells, (ii) breaking open or lysing the cells by mechanical, biological or chemical means, (iii) removing cell debris and insoluble materials e.g., by filtration or centrifugation, and (iv) optionally treating to remove residual RNA and DNA, but retaining the active enzymes and biosynthetic machinery for transcription and translation, and optionally the metabolic pathways for co-factor recycle, including but not limited to co-factors such as THF, S-adenosylmethionine, ATP, NADH, NAD and NADP and NADPH. In some embodiments, to produce a CFB reaction mixture, a cell extract or cell extracts may be supplemented to create a “supplemented cell extract” as described below.

As used herein, the term “supplemented cell extract” refers to a cell extract, used as part of a CFB reaction mixture, which is supplemented with all twenty proteinogenic naturally occurring amino acids and corresponding transfer ribonucleic acids (tRNAs), and optionally, may be supplemented with additional components, including but not limited to: (1) glucose, xylose, fructose, sucrose, maltose, or starch, (2) adenosine triphosphate (ATP), and/or adenosine diphosphate (ADP), purine and guanidine nucleotides, adenosine triphosphate, guanosine triphosphate, cytosine triphosphate, and/or uridine triphosphate, or combinations thereof, (3) cyclic-adenosine monophosphate (cAMP) and/or 3-phosphoglyceric acid (3-PGA), (4) nicotimamide adenine dinucleotides NADH and/or NAD, or nicotimamide adenine dinucleotide phosphates, NADPH, and/or NADP, or combinations thereof, (5) amino acid salts such as magnesium glutamate and/or potassium glutamate, (6) buffering agents such as HEPES, TRIS, spermidine, or phosphate salts, (7) inorganic salts, including but not limited to, potassium phosphate, sodium chloride, magnesium phosphate, and magnesium sulfate, (8) cofactors such as folinic acid and co-enzyme A (CoA), L(−)-5-formyl-5,6,7,8-tetrahydrofolic acid (THF), and/or biotin, (8) RNA polymerase, (9) 1,4-dithiothreitol (DTT), (10) magnesium acetate, and/or ammonium acetate, and/or (11) crowding agents such as PEG 8000, Ficoll 70, or Ficoll 400, or combinations thereof.

The terms “in vitro transcription and translation” and “TX-TL” are used interchangeably and refer to a cell-free biosynthesis process whereby biosynthetic genes, enzymes, and precursors are added to a cell-free biosynthesis system that possesses the machinery to carry out DNA transcription of genes or oligonucleotides leading to messenger ribonucleic acids (mRNA), and mRNA translation leading to proteins and peptides, including proteins that serve as enzymes to convert a lasso precursor peptide or lasso core peptide into a lasso peptide or lasso peptide analog. As used herein, the term “in vitro TX-TL machinery” refers to the components of a cell-free biosynthesis system that carry out DNA transcription of genes or oligonucleotides leading to messenger ribonucleic acids (mRNA), and mRNA translation leading to proteins and peptides.

The term “minimal set of lasso peptide biosynthesis components” as used herein refers to the minimum combination of components that is able to biosynthesize a lasso peptide without the help of any additional substance or functionality. The make-up of the minimal set of lasso peptide biosynthesis components may vary depending on the content and functionality of the components. Furthermore, the components forming the minimal set may present in varied forms, such as peptides, proteins, and nucleic acids.

The terms “analog” and “derivative” are used interchangeably to refer to a molecule such as a lasso peptide, that have been modified in some fashion, through chemical or biological means, to produce a new molecule that is similar but not identical to the original molecule.

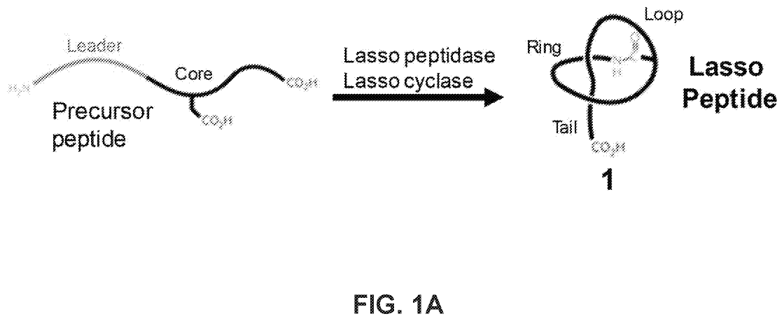

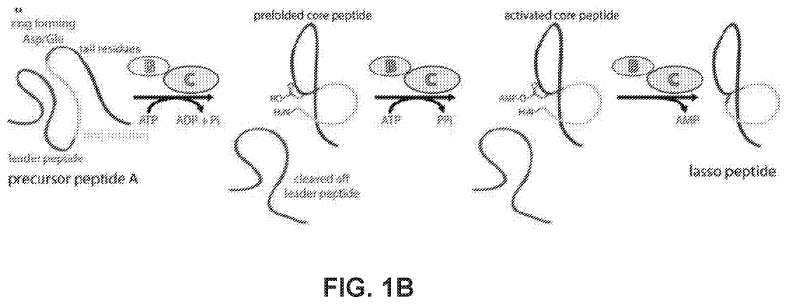

The term “lasso peptide” as used herein refers to a naturally-existing peptide or polypeptide having the general structure 1 as shown in FIG. 1A. In some embodiments, a lasso peptide is a peptide or polypeptide of at least eleven and up to about fifty amino acids sequence, which comprises an N-terminal core peptide, a middle loop region, and a C-terminal tail. The N-terminal core peptide forms a ring by cyclizing through the formation of an isopeptide bond between the N-terminal amino group of the core peptide and the side chain carboxyl groups of glutamate or aspartate residues located at positions 7, 8, or 9 of the core peptide, wherein the resulting macrolactam ring is formed around the C-terminal linear tail, which is threaded through the ring leading to the lasso (also referred to as lariat) topology held in place through sterically bulky side chains above and below the plane of the ring. In some embodiments, a lasso peptide contains one or more disulfide bond(s) formed between the tail and the ring. In some embodiments, a lasso peptide contains one or more disulfide bond(s) formed within the amino acid sequence of the tail.

The terms “lasso peptide analog” or “lasso peptide variant” are used herein interchangeably and refer to a derivative of a lasso peptide that has been modified or changed relative to its original structure or atomic composition. In various embodiments, the lasso peptide analog can (i) have at least one amino acid substitution(s), insertion(s) or deletion(s) as compared to the sequence of a lasso peptide; (ii) have at least one different modification(s) to the amino acids as compared to a lasso peptide, such modifications include but are not limited to acylation, biotinylation, O-methylation, N-methylation, amidation, glycosylation, esterification, halogenation, amination, hydroxylation, dehydrogenation, prenylation, lipidoylation, heterocyclization, phosphorylation; (iii) have at least one unnatural amino acid(s) as compared to the sequence of a lasso peptide; (iv) have at least one different isotope(s) as compared to the lasso peptide molecule; or any combination of (i) to (iv). As used herein, the term of “lasso peptide analog” also includes a conjugate or fusion made of a lasso peptide or a lasso peptide analog and one or more additional molecule(s). In some embodiments, the additional molecule can be another peptide or protein, including but not limited a lasso peptide and a cell surface receptor or an antibody or an antibody fragment. In some embodiments, the additional molecule can be a non-peptidic molecule, such as a drug molecule. In some embodiments, the lasso peptide analogs retain the same general lasso topology as shown in FIG. 1A. In some embodiments, production of a lasso peptide analog may occur by introducing a modification into the gene of a lasso precursor or core peptide, followed by transcription and translation and cyclization using CFB methods, as described herein, leading to a lasso peptide containing that modification. In an alternative embodiment, production of a lasso peptide analog may occur by introducing a modification into a lasso precursor or core peptide, followed by cyclization of each using CFB methods, as described herein, leading to a lasso peptide containing that modification. In another embodiment, production of a lasso peptide analog may occur by introducing a modification into a pre-formed lasso peptide, leading to a lasso peptide containing that modification.

The term “lasso peptide library” as used herein refers to a collection of at least two lasso peptides or lasso peptide analogs, or combinations thereof, which may be pooled together as a mixture or kept separated from one another. In some embodiments, the lasso peptide library is kept in vitro, such as in tubes or wells. In some embodiments, the lasso peptide library may be created by biosynthesis of at least two lasso peptides or lasso peptide variants using a CFB system. In some embodiments, the lasso peptides or lasso peptide variants of the library may be mixed with one or more component of the CFB system. In other embodiments, the lasso peptides or lasso peptide variants may be purified from the CFB system. In some embodiments, the lasso peptides or lasso peptide variants may be partially purified. In some embodiments, the lasso peptides or lasso peptide variants may be substantially purified. In some embodiments, the lasso peptides may be isolated. In some embodiments, the lasso peptide library may be created by isolating at least two lasso peptides from their natural environment. In some embodiments, the lasso peptides may be partially isolated. In some embodiments, the lasso peptides may be substantially isolated.

The term “isotopic variant” of a lasso peptide refers to a lasso peptide analog that contains an unnatural proportion of an isotope at one or more of the atoms that constitute such a peptide. In certain embodiments, an “isotopic variant” of a lasso peptide analog contains unnatural proportions of one or more isotopes, including, but not limited to, hydrogen (¹H), deuterium (²H), tritium (³H), carbon-11 (¹¹C), carbon-12 (¹²C) carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O) fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I) iodine-125 (¹²⁵I) iodine-127 (¹²⁷I) iodine-129 (¹²⁹I) and iodine-131 (¹³¹I). In certain embodiments, an “isotopic variant” of a lasso peptide is in a stable form, that is, non-radioactive. In certain embodiments, an “isotopic variant” of a lasso peptide contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O) oxygen-17 (¹⁷O), oxygen-18 (¹⁸O) fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a lasso peptide is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I) iodine-125 (¹²⁵I) iodine-129 (¹²⁹I) and iodine-131 (¹³¹I). It will be understood that, in a lasso peptide or lasso peptide analog as provided herein, any hydrogen can be ²H, as example, or any carbon can be ¹³C, as example, or any nitrogen can be ¹⁵N, as example, and any oxygen can be ¹⁸O, as example, where feasible according to the judgment of one of skill in the art. In certain embodiments, an “isotopic variant” of a lasso peptide contains an unnatural proportion of deuterium. Unless otherwise stated, structures of compounds (including peptides) depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

A “metabolic modification” refers to a biochemical reaction or biosynthetic pathway that is altered from its naturally-occurring state. Therefore, non-naturally occurring microorganisms can have genetic modifications to nucleic acids encoding metabolic polypeptides, or functional fragments thereof, which do not occur in the wild-type or natural organism.

As used herein, the term “isolated” when used in reference to a microbial organism or a biosynthetic gene, or a biosynthetic gene cluster, or a protein, or an enzyme, or a peptide, is intended to mean an organism, gene or biosynthetic gene cluster, protein, enzyme, or peptide that is substantially free of at least one component relative to the referenced microbial organism, gene, biosynthetic gene cluster, protein, enzyme, or peptide is found in nature or in its natural habitat. The term includes a microbial organism, gene, biosynthetic gene cluster, protein, enzyme, or peptide that is removed from some or all components as it is found in its natural environment. Therefore, an isolated microbial organism, gene, biosynthetic gene cluster, protein, enzyme, or peptide is partly or completely separated from other substances as it is found in nature or as it is grown, stored or subsisted in non-naturally occurring environments (e.g., laboratories). Specific examples of isolated microbial organisms, genes, biosynthetic gene clusters, proteins, enzymes, or peptides include partially pure microbes, genes, biosynthetic gene clusters, proteins, enzymes, or peptides, substantially pure microbes, genes biosynthetic gene clusters, proteins, enzymes, or peptides, and microbes cultured in a medium that is non-naturally occurring, or genes or biosynthetic gene clusters cloned in non-naturally occurring plasmids, or proteins, enzymes, or peptides purified from other components and substances present their natural environment, including other proteins, enzymes, or peptides.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “CoA” or “coenzyme A” is intended to mean an organic cofactor or prosthetic group (nonprotein portion of an enzyme) whose presence facilitates the activity of many enzymes (the apoenzyme) to form an active enzyme system. Coenzyme A functions in certain condensing enzymes, acts in acetyl or other acyl group transfer and in fatty acid synthesis and oxidation, pyruvate oxidation and in other acetylation.

As used herein, the term “substantially anaerobic” when used in reference to a culture or growth condition is intended to mean that the amount of oxygen is less than about 10% of saturation for dissolved oxygen in liquid media. The term also is intended to include sealed chambers of liquid or solid medium maintained with an atmosphere of less than about 1% oxygen.

The term “exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into a microbial organism or into a cell extract for cell-free expression. When used in reference to a biosynthetic activity, the term refers to an activity that is introduced into the host reference organism or into a cell extract for cell-free activity. The source can be, for example, a homologous or heterologous encoding nucleic acid that expresses the referenced activity following introduction into the host microbial organism or into a cell extract for cell-free expression of activity. Therefore, the term “endogenous” refers to a referenced molecule or activity that is present in a microbial host. Similarly, the term when used in reference to expression of an encoding nucleic acid refers to expression of an encoding nucleic acid contained within the microbial organism or into a cell extract. The term “heterologous” refers to a molecule or activity derived from a source other than the referenced species whereas “homologous” refers to a molecule or activity derived from the host microbial organism or organism used to produce a cell-flee extract. Accordingly, exogenous expression of an encoding nucleic acid of the invention can utilize either or both a heterologous or homologous encoding nucleic acid.

The term “stable,” as used herein, refers to compounds that are not substantially altered when subjected to conditions to allow for their production, detection, and, in certain embodiments, their recovery, purification, and use for one or more of the purposes disclosed herein.

The term “semi-synthesis” refers to modifying a natural material synthetically to create a new variant, derivative, or analog of the original natural material. For example, semisynthesis of a lasso peptide analog could involve chemical or enzymatic addition of biotin to an amino or sulfhydryl group on an amino acid side chain of a lasso peptide. The terms “derivative” or “analog” refer to a structural variant of compound that derives from a natural or non-natural material.

The terms “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of one enantiomer and about 5% or less of the other enantiomer based on the total weight of the racemate in question. In describing an optically active compound, the prefixes R and S are used to denote the absolute configuration of the molecule about its chiral center(s). The symbols (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “drug” and “therapeutic agent” refer to a compound, or a pharmaceutical composition thereof, which is administered to a subject for treating, preventing, or ameliorating one or more symptoms of a disorder, disease, or condition.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “therapeutically effective amount” are meant to include the amount of a therapeutic agent that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” refers an amount, concentration, or dosage of a compound that results in 50% inhibition of a maximal response in an assay that measures such response. The term “EC₅₀” refers an amount, concentration, or dosage of a compound that results in for 50% of a maximal response in an assay that measures such response. The term “CC₅₀” refers an amount, concentration, or dosage of a compound that results in 50% reduction of the viability of a host. In certain embodiments, the CC₅₀ of a compound is the amount, concentration, or dosage of the compound that that reduces the viability of cells treated with the compound by 50%, in comparison with cells untreated with the compound. The term “K_{d}” refers to the equilibrium dissociation constant for a ligand and a protein, which is measured to assess the binding strength that a small molecule ligand (such as a small molecule drug) has for a protein or receptor, such as a cell surface receptor. The dissociation constant, K_{d}, is commonly used to describe the affinity between a ligand and a protein or receptor; i.e., how tightly a ligand binds to a particular protein or receptor, and is the inverse of the association constant. Ligand-protein affinities are influenced by non-covalent intermolecular interactions between the two molecules such as hydrogen bonding, electrostatic interactions, hydrophobic and van der Waals forces. The analogous term “Kᵢ” is the inhibitor constant or inhibition constant, which is the equilibrium dissociation constant for an enzyme inhibitor, and provides an indication of the potency of an inhibitor.

As used herein, the phrase “biologically active” refers to a characteristic of any substance that has activity in a biological system and/or organism. For instance, a substance that, when administered to an organism, has a biological effect on that organism is considered to be biologically active. In particular embodiments, where a peptide or polypeptide is biologically active, a portion of that peptide or polypeptide that shares at least one biological activity of the peptide or polypeptide is typically referred to as a “biologically active” portion.

The terms “polypeptide” and “protein” are used interchangeably herein to refer to a polymer of greater than about fifty (50) amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “peptide” as used herein refers to a polymer chain containing between two and fifty (2-50) amino acid residues. The terms apply to naturally occurring amino acid polymers as well as amino acid polymers in which one or more amino acid residues is a non-naturally occurring amino acid, e.g., an amino acid analog or non-natural amino acid.

The term “amino acid” refers to naturally occurring and non-naturally occurring alpha-amino acids, as well as alpha-amino acid analogs and amino acid mimetics that function in a manner similar to the naturally occurring alpha-amino acids. Naturally encoded amino acids are the 22 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, pyrrolysine and selenocysteine). Amino acid analogs or derivatives refers to compounds that have the same basic chemical structure as a naturally occurring amino acid, i.e., a carbon that is bound to a hydrogen, a carboxyl group, an amino group, and a side chain R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “non-natural amino acid” or “non-proteinogenic amino acid” or “unnatural amino acid” refer to alpha-amino acids that contain different side chains (different R groups) relative to those that appear in the twenty-two common or naturally occurring amino acids listed above. In addition, these terms also can refer to amino acids that are described as having D-stereochemistry, rather than L-stereochemistry of natural amino acids, despite the fact that some amino acids do occur in the D-stereochemical form in Nature (e.g., D-alanine and D-serine).

The terms “oligonucleotide” and “nucleic acid” refer to oligomers of deoxyribonucleotides (e.g., DNA) or ribonucleotides (e.g., RNA) and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer, M. A., et al., Nucleic Acid Res., 1991, 19, 5081-1585; Ohtsuka, E. et al., J. Biol. Chem., 1985, 260, 2605-2608; and Rossolini, G. M., et al., Mol. Cell. Probes, 1994, 8, 91-98).

The term “antibody” describes an immunoglobulin whether natural or partly or wholly synthetically produced. The term also covers any peptide or protein having a binding domain which is, or is homologous to, an antigen binding domain. CDR grafted antibodies are also contemplated by this term. The term antibody as used herein will also be understood to mean one or more fragments of an antibody that retain the ability to specifically bind to an antigen, (Holliger, P. et al., Nature Biotech., 2005, 23 (9), 1126-1129). Non-limiting examples of such antibodies include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward, E. S., et al., Nature, 1989, 341, 544-546), which consists of a VH domain: and (vi) an isolated complementarity determining region (CDR). Furthermore, although the two domains of the Fv fragment, VL and VH, are coded for by separate genes, they are optionally joined, using recombinant methods, by a synthetic linker that enables them to be made as a single protein chain in which the VL and VH regions pair to form monovalent molecules (known as single chain Fv (scFv); see e.g., Bird, R E., et al., Science, 1988, 242, 423-426; Huston, J. S., et al., Proc. Natl. Acad. Sci. USA, 1988, 85, 5879-5883; and Osboum, J. K., et al., Nat. Biotechnol., 1998, 16, 778-781). Such single chain antibodies are also intended to be encompassed within the term antibody.

The term “assaying” is meant the creation of experimental conditions and the gathering of data regarding a particular result of the exposure to specific experimental conditions. For example, enzymes can be assayed based on their ability to act upon a detectable substrate. A lasso peptide can be assayed based on its ability to bind to a particular target molecule or molecules.

As used herein, the term “modulating” or “modulate” refers to an effect of altering a biological activity (i.e. increasing or decreasing the activity), especially a biological activity associated with a particular biomolecule such as a cell surface receptor. For example, an inhibitor of a particular biomolecule modulates the activity of that biomolecule, e.g., an enzyme, by decreasing the activity of the biomolecule, such as an enzyme. Such activity is typically indicated in terms of an inhibitory concentration (IC₅₀) of the compound for an inhibitor with respect to, for example, an enzyme.

As defined herein, the term “contacting” means that the compound(s) are combined and/or caused to be in sufficient proximity to particular other components, including, but not limited to, molecules, enzymes, peptides, oligonucleotides, complexes, cells, tissues, or other specified materials that potential binding interactions and/or chemical reaction between the compound and other components can occur.

It is understood that when more than one exogenous nucleic acid is included in a microbial organism or in a cell extract from a microbial organism that the more than one exogenous nucleic acids refer to the referenced encoding nucleic acid or biosynthetic activity, as discussed above. It is further understood, as disclosed herein, that such more than one exogenous nucleic acids can be introduced into the host microbial organism or into a cell extract, on separate nucleic acid molecules, on polycistronic nucleic acid molecules, or a combination thereof, and still be considered as more than one exogenous nucleic acid. For example, as disclosed herein, a microbial organism or a cell extract can be engineered to express two or more exogenous nucleic acids encoding a desired biosynthetic pathway enzyme, peptide, or protein. In the case where two exogenous nucleic acids encoding a desired activity are introduced into a host microbial organism or into a cell extract, it is understood that the two exogenous nucleic acids can be introduced as a single nucleic acid, for example, on a single plasmid or as linear strands of DNA, or on separate plasmids, or can be integrated into the host chromosome at a single site or multiple sites, and still be considered as two exogenous nucleic acids. Similarly, it is understood that more than two exogenous nucleic acids can be introduced into a host organism or into a cell extract in any desired combination, for example, on a single plasmid, or on separate plasmids, or as linear strands of DNA, or can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism or into a cell extract.

Those skilled in the art will understand that the genetic alterations, including metabolic modifications exemplified herein, are described with reference to a suitable host organism or a cell extract from a suitable host organism, such as E. coli and their corresponding metabolic reactions or a suitable source organism for desired genetic material such as genes, oligonucleotides, proteins, enzymes, and peptides for any desired metabolic pathways. However, given the complete genome sequencing of a wide variety of organisms and the high level of skill in the area of genomics, those skilled in the art will readily be able to apply the teachings and guidance provided herein to essentially all other organisms. For example, alterations to E. coli metabolic pathways and cell extracts derived thereof, and exemplified herein, can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less than 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism or cell extract. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease or the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms or cell extracts used in the invention having lasso peptide biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter off. Those skilled in the art will know what modifications can be made to the above parameters to either increase or decrease the stringency of the comparison, for example, and determine the relatedness of two or more sequences.

The term “partially” means that something takes place, as a function or activity, to provide the expected outcome or result in part and to a limited extent, not to the fullest extent. For example, if a lasso peptide is partially purified, the lasso peptide is isolated and purification steps afford the lasso peptide at purity level that is greater than about 20% and less than about 90%.

The term “substantially” means that something takes place, as a function or activity, to provide the expected outcome or result to a large degree and to a great extent, but still not to the fullest extent. For example, if a lasso peptide is substantially purified, the lasso peptide is isolated and purification steps afford the lasso peptide at purity level above 90% and as high as 99.99%.

The terms “plasmid” and “vector” are used interchangeably herein and refer to genetic constructs that incorporate genes of interest, along with regulatory components such as promoters, ribosome binding sites, and terminator sequences, along with a compatible origin of replication and a selectable marker (e.g., an antibiotic resistance gene), and which facilitate the cloning and expression of genes (e.g., from a lasso peptide biosynthetic pathway).

Provided herein are methods for the production of lasso peptides, lasso peptide analogs and lasso peptide libraries using cell-free biosynthesis systems and a minimal set of lasso peptide biosynthesis components. Also, provided herein are methods for the discovery of lasso peptides from Nature using cell-free biosynthesis systems and a minimal set of lasso peptide biosynthesis components. Also, provided herein are methods for the mutagenesis and production of lasso peptide variants using cell-flee biosynthesis systems and a minimal set of lasso peptide biosynthesis components. Also, provided herein are methods for optimization of lasso peptides using cell-flee biosynthesis systems and a minimal set of lasso peptide biosynthesis components.

The present invention provides herein methods for the synthesis of lasso peptides or lasso peptide analogs involving in vitro cell-free biosynthesis (CFB) systems that employ the enzymes and the biosynthetic and metabolic machinery present inside cells, but without using living cells. Cell-free biosynthesis systems provided herein for the production of lasso peptides and lasso peptide analogs have numerous applications for drug discovery. For example, cell-free biosynthesis systems allow rapid expression of natural biosynthetic genes and pathways and facilitate targeted or phenotypic activity screening of natural products, without the need for plasmid-based cloning or in vivo cellular propagation, thus enabling rapid process/product pipelines (e.g., creation of large lasso peptide libraries). A key feature of the CFB methods and systems provided herein for lasso peptide production is that oligonucleotides (linear or circular constructs of DNA or RNA) encoding a minimal set of lasso peptide biosynthesis pathway genes (e.g., lasso peptide genes A-C) may be added to a cell extract containing the biosynthetic machinery for transcribing and translating the minimal set of genes into the essential enzymes and lasso precursor peptides for production of lasso peptides and lasso peptide analogs.

Methods provided herein include cell-free (in vitro) biosynthesis (CFB) methods for making, synthesizing or altering the structure of lasso peptides. The CFB compositions, methods, systems, and reaction mixtures can be used to rapidly produce analogs of known compounds, for example lasso peptide analogs. Accordingly, the CFB methods can be used in the processes described herein that generate lasso peptide diversity. The CFB methods can produce in a CFB reaction mixture at least two or more of the altered lasso peptides to create a library of lasso peptides; preferably the library is a lasso peptide analog library, prepared, synthesized or modified by the CFB method or the present invention.

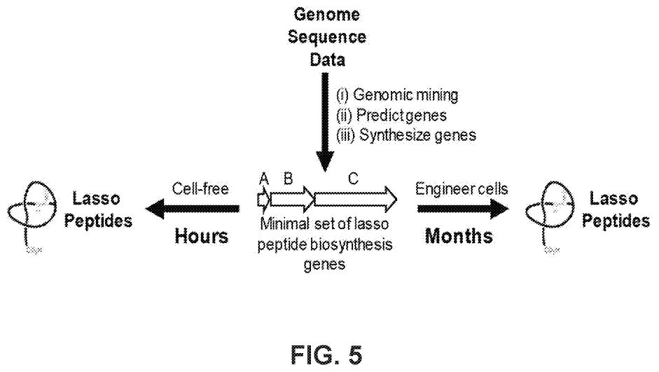

There are numerous benefits associated with using cell-free biosynthesis methods and systems for production of lasso peptides and lasso peptide analogs from a minimal set of lasso peptide biosynthesis components. When considering the analysis of large genomic databases that contain sequence information corresponding to lasso peptide biosynthetic genes and pathways, the minimal set of biosynthesis genes are predicted and then cloned, if the native organism is known and available. Alternatively, the minimal set of lasso peptide biosynthetic genes may be synthesized faster and cheaper as linear DNA or as plasmid-based genes. Production of a lasso peptide may then take place in cells, through cloning of the genes into a series of vectors in different configurations, followed by transformation of the vectors into appropriate host cells, growing the host cells with different vector configurations, and screening for host cells and conditions that lead to lasso peptide production. Cell-based production of lasso peptides can take months to enable. By contrast cell-free biosynthesis of lasso peptides requires no time-consuming cloning, plasmid propogation, transformation, in vivo selection or cell growth steps, but rather simply involves addition of the lasso peptide biosynthesis components (e.g., genes, as linear or circular DNA, or on plasmids), into a CFB reaction mixture containing supplemented cell extract, and lasso peptide production can occur in hours. Thus, one major benefit of cell-free biosynthesis of lasso peptides is speed (months for cell-based vs hours for cell-free). The specific lasso peptides and lasso peptide analogs formed when using the CFB methods and systems are defined by the input genes. Thus, CFB methods and systems for lasso peptide production, as described herein, lead only to formation of the desired lasso precursor peptides and lasso peptides of interest, which greatly facilitates isolation and purification of the desired lasso peptides and lasso peptide analogs. In addition, by using the CFB method, biosynthesis pathway flux to the target compound, such as lasso peptides, can be optimized by directing resources (e.g., carbon, energy, and redox sources) to production of the lasso peptides rather than supporting cellular growth and maintenance of the cells. Moreover, central metabolism, oxidative phosphorylation, and protein synthesis can be co-activated by the user, for example to recycle ATP, NADH, NADPH, and other co-factors, without the need to support cellular growth and maintenance. The lack of a cell wall precludes membrane transport limitations that can occur when using cells, provides for the ability to easily screen metabolites, proteins, and products (e.g., lasso peptides) by direct sampling, and also can allow production of products that ordinarily would be toxic or inhibitory to cell growth and survival. Finally, since no cells are involved, a cell-free biosynthesis processes can be conducted easily using liquid handling and robotic automation in order to enable high throughput biosynthesis of products, such as lasso peptides or lasso peptide analogs. FIG. 5 illustrates a comparison between cell-based and cell-free biosynthesis of lasso peptides.

### 5.3 Lasso Peptides

Bacterially-derived lasso peptides are emerging as a class of natural molecular scaffolds for drug design (Hegemann, J. D. et al., Acc. Chem. Res., 2015, 48, 1909-1919; Zhao, N., et al., Amino Acids, 2016, 48, 1347-1356; Maksimov, M. O., et al., Nat. Prod. Rep., 2012, 29, 996-1006). Lasso peptides are members of the larger class of natural ribosomally synthesized and post-translationally modified peptides (RiPPs). Lasso peptides are derived from a precursor peptide, comprising a leader sequence and core peptide sequence, which is cyclized through formation of an isopeptide bond between the N-terminal amino group of the linear core peptide and the side chain carboxyl groups of glutamate or aspartate residues located at positions 7, 8, or 9 of the linear core peptide. The resulting macrolactam ring is formed around the C-terminal linear tail, which is threaded through the ring leading to the characteristic lasso (also referred to as lariat) topology of general structure 1 as shown in FIG. 1, which is held in place through sterically bulky side chains above and below the plane of the ring, and sometimes containing disulfide bonds between the tail and the ring or alternatively only in the tail.

Lasso peptide gene clusters typically consist of three main genes, one coding for the precursor peptide (referred to as Gene A), and two for the processing enzymes, a lasso peptidase (referred to as Gene B) and a lasso cyclase (referred to as Gene C) that close the macrolactam ring around the tail to form the unique lariat structure. The precursor peptide consists of a leader sequence that binds to and directs the enzymes that carry out the cyclization reaction, and a core peptide sequence which contains the amino acids that together form the nascent lasso peptide upon cyclization. In addition, most lasso peptide gene clusters contain additional genes, such as those that encode for a small facilitator protein called a RIPP recognition element (RRE), those that encode for lasso peptide transporters, those that encode for kinases, or those that encode proteins that are believed to play a role in immunity, such as an isopeptidase (Burkhart, B. J., et al., Nat. Chem. Biol., 2015, 11, 564-570; Knappe, T. A. et al., J. Am. Chem. Soc., 2008, 130, 11446-11454; Solbiati, J. O. et al. J. Bacteriol., 1999, 181, 2659-2662; Fage, C D., et al., Angew. Chem. Int. Ed., 2016, 55, 12717-12721; Zhu, S., et al., J. Biol. Chem. 2016, 291, 13662-13678).

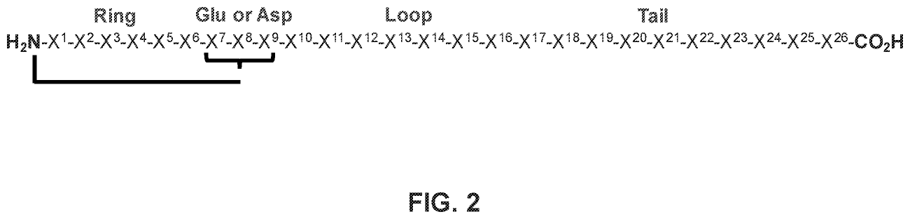

The ultimate lasso peptide directly derives from a core peptide that typically comprises a linear sequence ranging from about 11-50 amino acids in length. The macrolactam ring of a lasso peptide may contain 7, 8, or 9 amino acids, while the loop and tail vary in length. FIG. 2 shows an example of the general structure of a 26-mer linear core peptide corresponding to a lasso peptide.

Lasso peptides embody unique characteristics that are relevant to their potential utility as robust scaffolds for the development of drugs, agricultural and consumer products. Unique features of lasso peptides include: (1) small (1.5-3.0 kDa), compact, topologically unique and diverse structures, with rings, loops, folds, and tails that present amino acid residues in constrained conformations for receptor binding, (2) extraordinary stability against proteolytic degradation, high temperature, low pH and chemical denaturants; (3) gene-encoded lasso peptide precursor peptides; (4) gene clusters of bacterial origin allowing heterologous production in bacterial strains such as E. coli; (5) promiscuous biosynthetic machinery and lasso folding which tolerates amino acid substitutions at up to 80% of positions within the lasso peptide sequence, (6) ability to accept receptor epitope binding motifs grafted within the lasso structure in order to enhance potency and specificity for receptor binding, (7) ability to be further processed by biochemical or chemical means following lasso formation, and (8) ability to form fusion products using the free C-terminal tail of lasso peptides.

Historically, the barriers to lasso peptide development have included: (1) long, tedious, and costly extraction and fractionation processes for the discovery of new natural lasso peptides, (2) low yield or no production of lasso peptides by native hosts, (3) challenges associated with accurately predicting small lasso peptide gene clusters and precursor peptide genes within large genomic sequence datasets, (4) low throughput associated with cloning of lasso peptide biosynthetic gene clusters and poor yields in production of lasso peptides using common heterologous hosts, (5) lack of compelling demonstration of unique biological activities that address unmet needs, and (6) requirement for biosynthetic production of lasso peptides, which cannot be produced with the lasso topology by standard chemical peptide synthesis methods.

A genomic sequence mining algorithm called RODEO, has enabled identification of over 1300 entirely new lasso peptide gene clusters associated with a broad range of different bacterial species in the GenBank database, which is a vast increase over the 38 lasso peptides previously described in the literature (Tietz, J. I., et al., Nature Chem Bio, 2017, 13, 470-478). Previous genome mining tools struggled to identify lasso peptide biosynthetic gene clusters due to the small size of the gene clusters and particularly the precursor peptide genes (Hegemann, J. D., et al., Biopolymers, 2013, 100, 527-542; Maksimov, M O., et al., Proc. Nat. Acad Sci., 2012, 109, 15223-15228). This study also demonstrated that lasso peptides are much more widespread in Nature than previously expected.

A large percentage (>95%) of recently identified lasso peptide biosynthesis gene clusters have not been transformed into molecules, but rather remain as prophetic entities predicted on the basis of genome sequence analyses. Lasso peptide development is severely constrained by the lack of effective methods to rapidly convert virtual lasso peptide biosynthetic gene cluster sequences into actual molecules that can be characterized and screened for biological activity. Provided herein are methods and systems that enable the discovery, production, and optimization of lasso peptides and catalyze development of these unique peptide products for useful pharmaceutical, agricultural, and consumer applications.

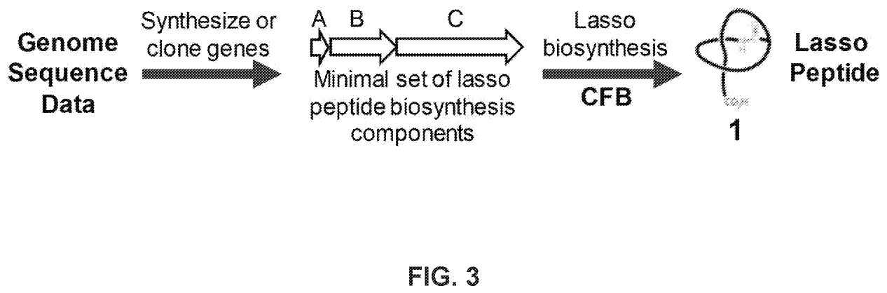

Naturally, lasso peptides are a unique class of ribosomally synthesized peptides produced by, for example, bacteria. In bacteria, lasso peptide gene clusters often include genes for functions such as transporters and immunity, which, in addition to the lasso biosynthesis pathway genes, are used for producing lasso peptides inside cells. These additional genes can be eliminated since transport, immunity, and other functions not directly linked to biosynthesis are superfluous in a cell-free system. Accordingly, systems and related methods of the present disclosure enable the rapid biosynthesis of lasso peptides from a minimal set of lasso peptide biosynthesis components (e.g., enzymes, proteins, peptides, genes and/or oligonucleotide sequences) using the in vitro cell-free biosynthesis (CFB) system as provided herein. Relative to lasso peptide production in cells, the use of a cell-free biosynthesis system not only simplifies the process, lowers cost, and greatly reduces the time for lasso peptide production and screening, but also enables the use of liquid handling and robotic automation in order to generate large libraries of lasso peptides and lasso peptide analogs in a high throughput manner. Additionally, the methods as provided herein enable the rapid evolution of lasso peptides to improve or optimize specific properties of interest, such as solubility, cell membrane permeability, metabolic stability, and pharmacokinetics. The present systems and methods thus enable the discovery and optimization of candidate lasso peptides and lasso peptide analogs for use in pharmaceutical, agricultural, and consumer applications. FIG. 3 shows the process of discovering lasso peptide encoding genes by genomic mining, and cell-free biosynthesis of lasso peptide.

### 5.4 Cell-Free Biosynthesis (CFB) Systems and Methods

In one aspect, provided herein are systems and related methods for producing lasso peptides or lasso peptide analogs through in vitro cell-free biosynthesis (CFB).

Cell-free methods, and especially cell-free protein synthesis methods, have been established and used as a technology to produce proteins froms single genes and to devise and prototype genetic circuits (Hodgman, C. E., Jewett, M. C., Metab. Eng., 2012, 14(3), 261-269). CFB methods and systems involve the production and/or use of at least two proteins or enzymes, which together interact and may serve as catalysts that lead to formation an independent third entity which is not a direct product of the input genes, but which is the final isolated product of interest. In a CFB method involving in vitro transcription and translation (TX-TL), protein or enzyme production can be accomplished directly from the corresponding oligonucleotides (RNA or DNA), including linear or plasmid-based DNA. The CFB methods and systems enable the user to modulate the concentrations of encoding DNA inputs in order to deliver individual pathway enzymes in the right ratios to optimally carry out production of a desired product. The ability to express multi-enzyme pathways using linear DNA in the CFB methods and systems bypasses the need for time-consuming steps such as cloning, in vivo selection, propagation of plasmids, and growth of host organisms. Linear DNA fragments can be assembled in 1 to 3 hours (hrs) via isothermal or Golden Gate assembly techniques and can be immediately used for a CFB reaction. The CFB reaction can take place to deliver a desired product in several hours, e.g. approximately 4-8 hours, or may be run for longer periods up to 48 hours. The use of linear DNA provides a valuable platform for rapidly prototyping libraries of DNA/genes. In the CFB methods and systems, mechanisms of regulation and transcription exogenous to the extract host, such as the tet repressor and T7 RNA polymerase, can be added as a supplement to CFB reaction mixtures and cell extracts in order to optimize the CFB system properties, or improve compound diversity or elevate production levels. The CFB methods and systems can be optimized to further enhance diversity and production of target compounds by modifying properties such as mRNA and DNA degradation rates, as well as proteolytic degradation of peptides and pathway enzymes. ATP regeneration systems that allow for the recycling of inorganic phosphate, a strong inhibitor of protein synthesis, also can be manipulated in the CFB methods and systems (Wang, Y., et al, BMC Biotechnology, 2009, 9:58 doi:10.1186/1472-6750-9-58). Redox co-factors and ratios, including e.g., NAD/NADH, NADP/NADPH, can be regenerated and controlled in CFB systems (Kay, J., et al., Metabolic Engineering, 2015, 32, 133-142).

As defined and used herein, cell-free biosynthesis methods and systems are to be distinguished from cell-free protein production systems. Cell-free protein production involves the addition of a single gene to a cell extract, whereby the gene is transcribed and translated to afford a single protein of interest, which is not necessarily catalytically active, and which is the final isolated product. Cell-free protein production methods have been used to produce: (1) proteins (Carlson, E. D., et al., Biotechnol. Adv., 2012, 30(5), 1185-1194; Swartz, J., et al., U.S. Pat. No. 7,338,789; Goerke, A. R., et al., U.S. Pat. No. 8,715,958), and (2) antibodies and antibody analogs (Zimmerman, E. S., et al., Bioconjugate Chem., 2014, 25, 351-361; Thanos, C. D., et al., US Patent No. 2015/0017187 A1).

By contrast, CFB methods involve the production and/or use of at least two proteins or enzymes, which together interact and may serve as catalysts that lead to formation an independent third entity, which is not a direct product of the input genes, but which is the final isolated product of interest. Cell-free biosynthesis methods involve the use of multistep biosynthesis pathways that may encompass: (i) the use of at least two isolated proteins or enzymes added to a CFB reaction mixture to produce a third independent product, (ii) the use of at least one gene and one protein or enzyme added to a CFB reaction mixture to produce a third independent product, or (iii) the use of at least two genes added to a CFB reaction mixture to produce a third independent product. The CFB methods (ii) and (iii) above involve the addition of genes to the CFB reaction mixture, and thus require the genes to undergo in vitro transcription and translation (TX-TL) to yield the peptides, proteins or enzymes to form the desired independent product of interest (e.g., a small molecule that is not a direct product of the input genes). CFB processes recently have been used for the production of small molecules (1,3-Butanediol-Kay, J., et al., Metabolic Engineering, 2015, 32, 133-142; Carbapenem-Blake, W. J., et al., U.S. Pat. No. 9,469,861). However, these reports do not implement CFB methods involving TX-TL, and cell-free biosynthesis methods involving TX-TL have not been implemented for the production of lasso peptides or lasso peptide analogs using a minimal set of lasso peptide biosynthesis components, as described herein.

In some embodiments, for the CFB methods to function, the expressed enzymes in the CFB system fold and function properly with other additional components (e.g., trace metals, chaperons, precursors, recycled co-factors, and recycled energy molecules) for the biosynthetic pathway to form the desired product. In some embodiments, a CFB reaction mixtures comprise optimized cell extracts that provide these components along with the transcription and translation machinery that: (i) accepts the accessible oligonucleotide codon usage (e.g., GC content >60%), and (ii) can transcribe small and large genes (e.g., >3 kilobases) and translate and properly fold small and large proteins (e.g., >100 kDa). Most cell extracts described in the literature or available commercially for in vitro expression have been optimized for cell-free protein synthesis, not for cell-free biosynthesis (Hoffmann, M., et al., Biotech. Ann. Rev., 2004, 10, 1-29; Gagoski, D., et al., Biotechnol. Bioeng., 2016; 113: 292-300; Shimizu, Y., et al., Cell-Free Protein Production: Methods and Protocols, in Methods in Molecular Biology, Y. Endo et al. (eds.), vol. 607, Chapter 2, pp 11-21, Springer New York, 2010; Takai, K, et al., Nature Protocols, 2010, 5, 227-238; Li, J., et al., PLoS ONE, 2014, 9, e106232. doi:10.1371/journal.pone.0106232; Kigawa, T., et al., J. Struct. Functional Genomics, 2004, 5, 63-68; see also website of Promega Corporation (Fitchburg Center, Wis., USA) at www.promega.com). Descriptions and comparisons of the performance of cell extracts derived from different cell types have been reported (Carlson, E. D., et al., Biotechnol. Adv., 2012, 30(5), 1185-1194; Gagoski, D., et al., Biotechnol. Bioeng., 2016; 113: 292-300).

CFB methods and systems provided herein for the synthesis of lasso peptides and lasso peptide analogs from a minimal set of lasso peptide biosynthesis components, are conducted in a CFB reaction mixture, comprising one or more cell extracts that are supplemented with all twenty proteinogenic naturally occurring amino acids and corresponding transfer ribonucleic acids (tRNAs). Cell extracts used in the CFB reaction mixture, provided herein for the synthesis of lasso peptides and lasso peptide analogs from a minimal set of lasso peptide biosynthesis components also may be supplemented with additional components, including but not limited to, glucose, xylose, fructose, sucrose, maltose, starch, adenosine triphosphate (ATP), and/or adenosine diphosphate (ADP), purine and guanidine nucleotides, adenosine triphosphate, guanosine triphosphate, cytosine triphosphate, and uridine triphosphate, cyclic-adenosine monophosphate (cAMP) and/or 3-phosphoglyceric acid (3-PGA), nicotimamide adenine dinucleotides NADH and/or NAD, or nicotimamide adenine dinucleotide phosphates, NADPH, and/or NADP, or combinations thereof, amino acid salts such as magnesium glutamate and/or potassium glutamate, buffering agents such as HEPES, TRIS, spermidine, or phosphate salts, inorganic salts, including but not limited to, potassium phosphate, sodium chloride, magnesium phosphate, and magnesium sulfate, folinic acid and co-enzyme A (CoA), crowding agents such as PEG 8000, Ficoll 70, or Ficoll 400, L(−)-5-formyl-5,6,7,8-tetrahydrofolic acid, RNA polymerase, biotin, 1,4-dithiothreitol (DTT), magnesium acetate, ammonium acetate, or combinations thereof. For a general description of cell-free extract production and preparation. (Krinsky, N., et al., PLoS ONE, 2016, 11(10): e0165137).

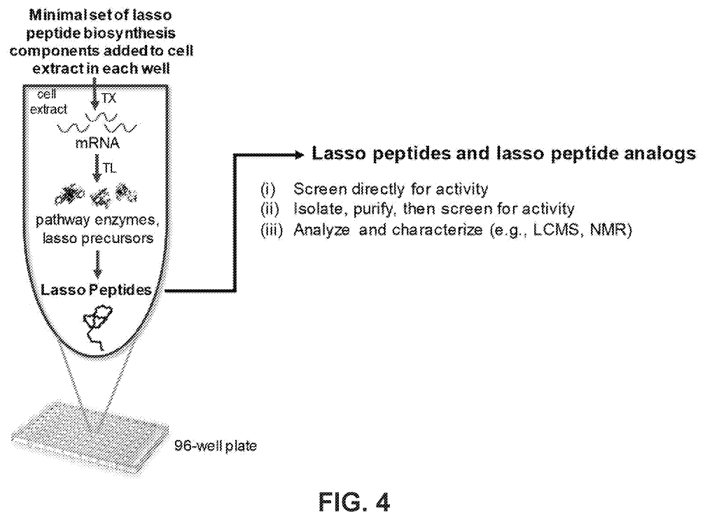

In some embodiments, the CFB system employs the enzymes, and the biosynthetic and metabolic machinery of a cell, without using a living cell. The present CFB systems and related methods provided herein for the production of lasso peptides and lasso peptide analogs have numerous applications for drug discovery involving rapid expression of lasso peptide biosynthetic genes and pathways and by allowing targeted or phenotypic activity screening of lasso peptides and lasso peptide analogs, without the need for plasmid-based cloning or in vivo cellular propagation, thus enabling rapid process/product pipelines (e.g., creation of large lasso peptide libraries). The CFB methods and systems provided herein for lasso peptide production have the feature that oligonucleotides (linear or circular constructs of DNA or RNA) encoding a minimal set of lasso peptide biosynthetic pathway genes (e.g., Genes A-C) may be added to a cell extract containing the biosynthetic machinery for transcribing and translating the genes into precursor peptide and the enzymes for processing the lasso precursor peptide into a lasso peptide. By using a CFB system, biosynthesis pathway flux to the target compound can be optimized by directing resources (e.g., carbon, energy, and redox sources) to user-defined objectives. Thus, central metabolism, oxidative phosphorylation, and protein synthesis can be co-activated by the user without the need to support cellular growth and maintenance. The lack of a cell wall also provides for the ability to easily screen metabolites, proteins, and products (e.g., lasso peptides) that are toxic or inhibitory to cell growth and survival. Finally, since no cells are involved, cell-free biosynthesis reactions or processes can be conducted using liquid handling and robotic automation in order to enable high throughput synthesis of products, such as lasso peptide and lasso peptide analog libraries. FIG. 4 illustrates cell-free biosynthesis of lasso peptides using in vitro transcription/translation, and construction of a lasso peptide library for screening of activities.

In certain embodiments, cell-free biosynthesis methods and systems described herein are used to produce lasso peptides and lasso peptide analogs by combining and contacting a minimal set of lasso peptide biosynthesis components, including, for example: (1) isolated precursor peptides or precursor peptide fusions, combined together and contacted with isolated proteins that include a lasso peptidase and a lasso cyclase, or fusions thereof, (2) oligonucleotides (linear or circular constructs of DNA or RNA) that encode for precursor peptides or precursor peptide fusions, combined together and contacted with isolated proteins that include a lasso peptidase and a lasso cyclase, or fusions thereof, (3) isolated precursor peptides or precursor peptide fusions, combined together and contacted with oligonucleotides that encode for a lasso peptidase and a lasso cyclase, or fusions thereof, (4) oligonucleotides that encode for precursor peptides, a lasso peptidase, and a lasso cyclase, or fusions thereof, combined together and contacted, (5) isolated core lasso peptides combined and contacted with isolated lasso cyclases, or fusions thereof, (6) oligonucleotides that encode for core lasso peptides combined and contacted with isolated lasso cyclases, or fusions thereof, or (7) oligonucleotides that encode for core lasso peptides combined and contacted with oligonucleotides that encode for lasso cyclases, or fusions thereof, in a cell-free reaction mixture.

In some embodiments, the CFB system comprises the biosynthetic and metabolic machinery of a cell, without using a living cell. In some embodiments, the CFB system comprises a CFB reaction mixture as provided herein. In some embodiments, the CFB system comprises a cell extract as provided. In some embodiments, the cell extract is derived from prokaryotic cells. In some embodiments, the cell extract is derived from eukaryotic cells. In some embodiments, the CFB system comprises a supplemented cell extract provided herein. In some embodiments, the CFB system comprises in vitro transcription and translation machinery as provided herein.

In some embodiments, the CFB system comprises a minimal set of lasso peptide biosynthesis components. In some embodiments, the minimal set of lasso peptide biosynthesis components are capable of producing a lasso peptide or a lasso peptide analog of interest without the help of any additional substance of functionality. In some embodiments, the minimal set of lasso peptide biosynthesis components comprises at least one component that functions to provide a lasso precursor peptide and at least one component that functions to process the lasso precursor peptide into a lasso peptide or a lasso peptide analog. In some embodiments, the minimal set of lasso peptide biosynthesis components comprises at least one component that functions to provide a lasso core peptide and at least one component that functions to process the lasso core peptide into a lasso peptide or a lasso peptide analog.

In some embodiments, the CFB system comprises a minimal set of lasso peptide biosynthesis components. In particular embodiments, the minimal set of lasso peptide biosynthesis components comprises at least one component that functions to produce a lasso precursor peptide. In particular embodiments, the minimal set of lasso peptide biosynthesis components comprises at least one component that functions to produce a lasso core peptide. In particular embodiments, the minimal set of lasso peptide biosynthesis components comprises at least one component that functions to produce a lasso peptidase. In particular embodiments, the minimal set of lasso peptide biosynthesis components comprises at least one component that functions to produce a lasso cyclase. In particular embodiments, the minimal set of lasso peptide biosynthesis components comprises at least one component that functions to produce a RIPP recognition element (RRE). In particular embodiments, the minimal set of lasso peptide biosynthesis components comprises at least one component that functions to produce (i) a lasso precursor peptide, (ii) a lasso peptidase, and (iii) a lasso cyclase. In particular embodiments, the minimal set of lasso peptide biosynthesis components comprises at least one component that functions to produce (i) a lasso precursor peptide, (ii) a lasso peptidase, (iii) a lasso cyclase, and (iv) an RRE. In particular embodiments, the minimal set of lasso peptide biosynthesis components comprises at least one component that functions to produce (i) a lasso core peptide, and (ii) a lasso cyclase. In particular embodiments, the minimal set of lasso peptide biosynthesis components comprises at least one component that functions to produce (i) a lasso core peptide, (ii) a lasso cyclase; and (iii) an RRE.

In some embodiments, the component functions to produce a peptide or polypeptide (e.g., a lasso precursor peptide, a lasso peptidase, or a lasso cyclase) in the minimal set of lasso peptide biosynthesis components comprises the peptide or polypeptide to be produced. In some embodiments, the component functions to produce a peptide or polypeptide (e.g., a lasso precursor peptide, a lasso peptidase, or a lasso cyclase) in the minimal set of lasso peptide biosynthesis components comprises a polynucleotide encoding such peptide or polypeptide. In some embodiments, the component functions to produce a peptide or polypeptide (e.g., a lasso precursor peptide, a lasso peptidase, or a lasso cyclase) in the minimal set of lasso peptide biosynthesis components is the peptide or polypeptide to be produced. In some embodiments, the component functions to produce a peptide or polypeptide (e.g., a lasso precursor peptide, a lasso peptidase, or a lasso cyclase) in the minimal set of lasso peptide biosynthesis components is a polynucleotide encoding such peptide or polypeptide. In some embodiments, the component functions to produce a peptide or polypeptide (e.g., a lasso precursor peptide, a lasso peptidase, or a lasso cyclase) in the minimal set of lasso peptide biosynthesis components comprises a polynucleotide encoding such peptide or polypeptide, and the minimal set of lasso peptide biosynthesis components further comprises in vitro TX-TL machinery capable of producing such peptide or polypeptide from the polynucleotide encoding such peptide or polypeptide.

In certain embodiments, the CFB systems described herein are used to produce lasso peptides and lasso peptide analogs by combining and contacting a minimal set of lasso peptide biosynthesis components, including, for example: (1) isolated precursor peptides or precursor peptide fusions, combined together and contacted with isolated proteins that include a lasso peptidase and a lasso cyclase, or fusions thereof, (2) oligonucleotides (linear or circular constructs of DNA or RNA) that encode for precursor peptides or precursor peptide fusions, combined together and contacted with isolated proteins that include a lasso peptidase and a lasso cyclase, or fusions thereof, (3) isolated precursor peptides or precursor peptide fusions, combined together and contacted with oligonucleotides that encode for a lasso peptidase and a lasso cyclase, or fusions thereof, (4) oligonucleotides that encode for precursor peptides, a lasso peptidase, and a lasso cyclase, or fusions thereof, combined together and contacted, (5) isolated core lasso peptides combined and contacted with isolated lasso cyclases, or fusions thereof, (6) oligonucleotides that encode for core lasso peptides combined and contacted with isolated lasso cyclases, or fusions thereof, or (7) oligonucleotides that encode for core lasso peptides combined and contacted with oligonucleotides that encode for lasso cyclases, or fusions thereof, in a cell-free reaction mixture.

Particularly, in some embodiments, the CFB system comprises one or more components that function to provide a lasso precursor peptide. In some embodiments, the one or more components that function to provide the lasso precursor peptide comprise the lasso precursor peptide. In some embodiments, the one or more components that function to provide the lasso precursor peptide comprise a nucleic acid encoding the lasso precursor peptide and in vitro TX-TL machinery.

In some embodiments, the CFB system comprises one or more components that function to provide a lasso peptidase. In some embodiments, the one or more components that function to provide the lasso peptidase comprise the lasso peptidase. In some embodiments, the one or more components that function to provide the lasso peptidase comprise a nucleic acid encoding the lasso peptidase and in vitro TX-TL machinery.

In some embodiments, the CFB system comprises one or more components that function to provide a lasso cyclase. In some embodiments, the one or more components that function to provide the lasso cyclase comprise the lasso cyclase. In some embodiments, the one or more components that function to provide the lasso cyclase comprise a nucleic acid encoding the lasso cyclase and in vitro TX-TL machinery.

In some embodiments, the CFB system comprises one or more components that function to provide a RIPP recognition element (RRE). In some embodiments, the one or more components that function to provide the RRE comprise the RRE. In some embodiments, the one or more components that function to provide the lasso cyclase comprise a nucleic acid encoding the RRE and in vitro TX-TL machinery.

In some embodiments, the CFB system comprises one or more components that function to provide a lasso core peptide. In some embodiments, the one or more components that function to provide the lasso core peptide comprise the lasso core peptide. In some embodiments, the one or more components that function to provide the lasso core peptide comprise a nucleic acid encoding the lasso core peptide and in vitro TX-TL machinery.

In some embodiments, the CFB system comprises (i) a nucleic acid encoding the lasso precursor peptide; (ii) a nucleic acid encoding the lasso peptidase; (iii) a nucleic acid encoding the lasso cyclase; and (iv) in vitro TX-TL machinery. In some embodiments, the CFB system comprises (i) a nucleic acid encoding the lasso precursor peptide; (ii) a lasso peptidase; (iii) a nucleic acid encoding the lasso cyclase; and (iv) in vitro TX-TL machinery. In some embodiments, the CFB system comprises (i) a nucleic acid encoding the lasso precursor peptide; (ii) a nucleic acid encoding the lasso peptidase; (iii) a lasso cyclase; and (iv) in vitro TX-TL machinery. In some embodiments, the CFB system comprises (i) a nucleic acid encoding the lasso precursor peptide; (ii) a lasso peptidase; (iii) a lasso cyclase; and (iv) in vitro TX-TL machinery. In some embodiments, the CFB system comprises (i) a lasso precursor peptide; (ii) a lasso peptidase; and (iii) a lasso cyclase. In some embodiments, the CFB system comprises (i) a precursor peptide; (ii) a lasso peptidase; (iii) a nucleic acid encoding the lasso cyclase; and (iv) in vitro TX-TL machinery. In some embodiments, the CFB system comprises (i) a lasso precursor peptide; (ii) a nucleic acid encoding the lasso peptidase; (iii) a lasso cyclase; and (iv) in vitro TX-TL machinery. In some embodiments, the CFB system comprises (i) a lasso precursor peptide; (ii) a nucleic acid encoding the lasso peptidase; (iii) a nucleic acid encoding the lasso cyclase; and (iv) in vitro TX-TL machinery.

In some embodiments, the CFB system comprises (i) a nucleic acid encoding the lasso precursor peptide; (ii) a nucleic acid encoding the lasso peptidase; (iii) a nucleic acid encoding the lasso cyclase; (iv) a nucleic acid encoding the RRE; and (v) in vitro TX-TL machinery. In some embodiments, the CFB system comprises (i) a nucleic acid encoding the lasso precursor peptide; (ii) a lasso peptidase; (iii) a nucleic acid encoding the lasso cyclase; (iv) a nucleic acid encoding the RRE; and (v) in vitro TX-TL machinery. In some embodiments, the CFB system comprises (i) a nucleic acid encoding the lasso precursor peptide; (ii) a nucleic acid encoding the lasso peptidase; (iii) a lasso cyclase; (iv) a nucleic acid encoding the RRE; and (v) in vitro TX-TL machinery. In some embodiments, the CFB system comprises (i) a nucleic acid encoding the lasso precursor peptide; (ii) a nucleic acid encoding the lasso peptidase; (iii) a nucleic acid encoding the lasso cyclase; (iv) a RRE; and (v) in vitro TX-TL machinery. In some embodiments, the CFB system comprises (i) a nucleic acid encoding the lasso precursor peptide; (ii) a lasso peptidase; (iii) a lasso cyclase; (iv) a nucleic acid encoding the RRE; and (v) in vitro TX-TL machinery. In some embodiments, the CFB system comprises (i) a nucleic acid encoding the lasso precursor peptide; (ii) a lasso peptidase; (iii) a nucleic acid encoding the lasso cyclase; (iv) a RRE; and (v) in vitro TX-TL machinery. In some embodiments, the CFB system comprises (i) a nucleic acid encoding the lasso precursor peptide; (ii) a nucleic acid encoding the lasso peptidase; (iii) a lasso cyclase; (iv) a RRE; and (v) in vitro TX-TL machinery. In some embodiments, the CFB system comprises (i) a nucleic acid encoding the lasso precursor peptide; (ii) a lasso peptidase; (iii) a lasso cyclase; (iv) a RRE; and (v) in vitro TX-TL machinery. In some embodiments, the CFB system comprises (i) a lasso precursor peptide; (ii) a nucleic acid encoding the lasso peptidase; (iii) a nucleic acid encoding the lasso cyclase; (iv) a nucleic acid encoding the RRE; and (v) in vitro TX-TL machinery. In some embodiments, the CFB system comprises (i) a lasso precursor peptide; (ii) a lasso peptidase; (iii) a nucleic acid encoding the lasso cyclase; (iv) a nucleic acid encoding the RRE; and (v) in vitro TX-TL machinery. In some embodiments, the CFB system comprises (i) a lasso precursor peptide; (ii) a nucleic acid encoding the lasso peptidase; (iii) a lasso cyclase; (iv) a nucleic acid encoding the RRE; and (v) in vitro TX-TL machinery. In some embodiments, the CFB system comprises (i) a lasso precursor peptide; (ii) a nucleic acid encoding the lasso peptidase; (iii) a nucleic acid encoding the lasso cyclase; (iv) a RRE; and (v) in vitro TX-TL machinery. In some embodiments, the CFB system comprises (i) a lasso precursor peptide; (ii) a lasso peptidase; (iii) a lasso cyclase; (iv) a nucleic acid encoding the RRE; and (v) in vitro TX-TL machinery. In some embodiments, the CFB system comprises (i) a lasso precursor peptide; (ii) a lasso peptidase; (iii) a nucleic acid encoding the lasso cyclase; (iv) a RRE; and (v) in vitro TX-TL machinery. In some embodiments, the CFB system comprises (i) a lasso precursor peptide; (ii) a nucleic acid encoding the lasso peptidase; (iii) a lasso cyclase; (iv) a RRE; and (v) in vitro TX-TL machinery. In some embodiments, the CFB system comprises (i) a lasso precursor peptide; (ii) a lasso peptidase; (iii) a lasso cyclase; and (iv) a RRE.

In some embodiments, the CFB system comprises (i) a nucleic acid encoding the lasso core peptide; (ii) a nucleic acid encoding the lasso cyclase; and (iii) in vitro TX-TL machinery. In some embodiments, the CFB system comprises (i) a nucleic acid encoding the lasso core peptide; (ii) a lasso cyclase; and (iii) in vitro TX-TL machinery. In some embodiments, the CFB system comprises (i) a lasso core peptide; (ii) a nucleic acid encoding the lasso cyclase; and (iii) in vitro TX-TL machinery. In some embodiments, the CFB system comprises (i) a lasso core peptide; and (ii) a cyclase.

In some embodiments, the CFB system comprises (i) a nucleic acid encoding the lasso precursor peptide; (ii) a nucleic acid encoding the lasso cyclase; (iii) a nucleic acid encoding the RRE; and (iv) in vitro TX-TL machinery. In some embodiments, the CFB system comprises (i) a nucleic acid encoding the lasso precursor peptide; (ii) a lasso cyclase; (iii) a nucleic acid encoding the RRE; and (iv) in vitro TX-TL machinery. In some embodiments, the CFB system comprises (i) a nucleic acid encoding the lasso precursor peptide; (ii) a nucleic acid encoding the lasso cyclase; (iii) a RRE; and (iv) in vitro TX-TL machinery. In some embodiments, the CFB system comprises (i) a nucleic acid encoding the lasso precursor peptide; (ii) a lasso cyclase; (iii) a RRE; and (iv) in vitro TX-11_, machinery. In some embodiments, the CFB system comprises (i) a lasso precursor peptide; (ii) a nucleic acid encoding the lasso cyclase; (iii) a nucleic acid encoding the RRE; and (iv) in vitro TX-TL machinery. In some embodiments, the CFB system comprises (i) a lasso precursor peptide; (ii) a lasso cyclase; (iii) a nucleic acid encoding the RRE; and (iv) in vitro TX-TL machinery. In some embodiments, the CFB system comprises (i) a lasso precursor peptide; (ii) a nucleic acid encoding the lasso cyclase; (iii) a RRE; and (iv) in vitro TX-TL machinery. In some embodiments, the CFB system comprises (i) a lasso precursor peptide; (ii) a lasso cyclase; and (iii) a RRE.

In some embodiments, the CFB system comprises one or more gene(s) of a lasso peptide gene cluster, or protein coding fragment thereof, or encoded product thereof. In some embodiments, the protein coding fragment is an open reading frame. In some embodiments, the CFB system comprises components that function to provide (i) at least one lasso precursor peptide having an amino acid sequence selected from the even number of SEQ ID Nos: 1-2630, or the corresponding core peptide fragment thereof (ii) at least one lasso peptidase having an amino acid sequence selected from peptide Nos: 1316-2336; (iii) at least one lasso cyclase having an amino acid sequence selected from peptide Nos: 2337-3761; (iv) at least one RRE having nucleic acid sequence selected from peptide Nos: 3762-4593; or (v) any combinations of (i) through (iv). In particular embodiments, the CFB system comprises components that function to provide at least one combination of one or more selected from a lasso precursor peptide, a lasso peptidase, a lasso cyclase and a RRE as shown in Table 2. In some embodiments, the components of a CFB system that function to provide a peptide or polypeptide having the amino acid sequence selected from peptide Nos: 1-4593 comprise the peptide or polypeptide having the amino acid sequence selected from peptide Nos: 1-4593 themselves. In other embodiments, the components of a CFB system that function to provide a peptide or polypeptide having the amino acid sequence selected from peptide Nos: 1-4593 comprises a polynucleotide encoding the peptide or polypeptide having the amino acid sequence selected from peptide Nos: 1-4593. Non-limiting examples of genomic sequences from specified microbial species that encode for the amino acid sequences having peptide Nos: 1-4593 are provided in Tables 3, 4 and 5, and the even numbers of SEQ ID Nos: 1-2630. Further, those skilled in the art would be readily capable of identifying and/or recognizing additional coding nucleic acid sequences, either synthetic or naturally-occurring in the same or different microbial organism as disclosed herein, using genetic tools well-known in the art.

In some embodiments, the CFB system comprises one or more components function to provide a fusion protein. In some embodiments, the one or more components function to provide the fusion protein comprise the fusion protein. In some embodiments, the one or more components function to provide the fusion protein comprise a polynucleotide encoding the fusion protein.

In some embodiments, the fusion protein comprised a lasso precursor peptide or a lasso core peptide fused to one or more additional peptide or polypeptide. In some embodiments, the one or more additional peptide or polypeptide is fused to the N-terminus of the lasso precursor peptide or lasso core peptide. In some embodiments, the one or more additional peptide or polypeptide is fused at the C-terminus of the lasso precursor peptide or lasso core peptide. In some embodiments, a polynucleotide encoding the fusion protein comprises a nucleic acid sequence encoding the lasso precursor peptide or the lasso core peptide, wherein the 5′ end of the nucleic acid sequence is linked to a nucleic acid sequence encoding the one or more additional peptide or polypeptide. In some embodiments, a polynucleotide encoding the fusion protein comprises a nucleic acid sequence encoding the lasso precursor peptide or the lasso core peptide, wherein the 3′ end of the nucleic acid sequence is linked to a nucleic acid sequence encoding the one or more additional peptide or polypeptide. In some embodiments, the fusion protein comprises an amino acid linker between the lasso precursor peptide or lasso core peptide and the one or more additional peptide or polypeptide. In some embodiments, the fusion protein does not comprise an amino acid linker between the lasso precursor peptide or lasso core peptide and the one or more additional peptide or polypeptide.

In some embodiments, the fusion protein comprised a lasso precursor peptide or a lasso core peptide fused to one or more additional peptide or polypeptide. In some embodiments, the one or more additional peptide or polypeptide comprises a peptide or polypeptide encoded by a lasso peptide gene cluster. Examples of peptide or polypeptide that can be fused with a lasso precursor peptide or a lasso core peptide according to the present disclosure include but are not limited to (i) a lasso precursor peptide, (ii) a lasso core peptide; (iii) a lasso peptidase; (iv) a lasso cyclase; (v) a RRE; or (vi) any combinations of (i) to (vi). In specific embodiments, the fusion protein comprises a lasso precursor peptide fused to a RRE. In specific embodiments, the fusion protein comprises a lasso core peptide fused to a RRE. In specific embodiments, the fusion protein comprises multiple lasso precursor peptides and/or lasso core peptides. In specific embodiments, at least one of the multiple lasso precursor peptides and/or lasso core peptides is different from another of the multiple lasso precursor peptide and/or lasso core peptide.

In some embodiments, the fusion protein comprised a lasso precursor peptide or a lasso core peptide fused to one or more additional peptide or polypeptide. In some embodiments, the one or more additional peptide or polypeptide comprises a peptide or polypeptide that facilitates production of the lasso precursor peptide or lasso core peptide or the lasso peptide derived therefrom through cell-free biosynthesis. Examples of peptide or polypeptide that can be fused with a lasso precursor peptide or a lasso core peptide according to the present disclosure include but are not limited to (i) a peptide or polypeptide that increases the level of transcription of the lasso precursor peptide or lasso core peptide in the CFB system; (ii) a peptide or polypeptide that increases the level of translation of the lasso precursor peptide or lasso core peptide in the CFB system; (iii) a peptide or polypeptide that facilitates the processing of the lasso precursor peptide or lasso core peptide into the lasso peptide; (iv) a peptide or polypeptide that improves stability of the lasso precursor peptide or lasso core peptide or the lasso peptide derived therefrom; (v) a peptide or polypeptide that improves solubility of the lasso precursor peptide or lasso core peptide or the lasso peptide derived therefrom; (vi) a peptide or polypeptide that enables or facilitates the detection of the lasso precursor peptide or lasso core peptide or the lasso peptide derived therefrom; (vii) a peptide or polypeptide that enables or facilitates purification of the lasso precursor peptide or lasso core peptide or the lasso peptide derived therefrom; (viii) a peptide or polypeptide that enables or facilitates immobilization of the lasso precursor peptide or lasso core peptide or the lasso peptide derived therefrom; or (ix) any combination of (i) to (viii).

In some embodiments, the fusion protein comprised a lasso precursor peptide or a lasso core peptide fused to one or more additional peptide or polypeptide. In some embodiments, the one or more additional peptide or polypeptide comprises a biologically active peptide or polypeptide. Examples of biologically active peptide or polypeptide that can be fused with a lasso precursor peptide or lasso core peptide according to the present disclosure include but are not limited to (i) a peptide or polypeptide capable of binding to a target molecule (e.g., an antibody or an antigen); (ii) a peptide or polypeptide that enhance cell permeability of the fusion protein; (iii) a peptide or polypeptide capable of conjugating the fusion protein to at least one additional copy of the fusion protein; (iv) a peptide or polypeptide capable of linking the fusion protein to one or more peptidic or non-peptidic molecule; (v) a peptide or polypeptide capable of modulating activity of the lasso precursor peptide or lasso core peptide; (vi) a peptide or polypeptide capable of modulating activity of the lasso peptide derived from the lasso precursor peptide or the lasso core peptide; or (vii) any combinations of (i) to (vi).

In some embodiments, the fusion protein comprised a lasso peptidase or a lasso cyclase fused to one or more additional peptide or polypeptide. In some embodiments, the one or more additional peptide or polypeptide is fused to the N-terminus of the lasso peptidase or the lasso cyclase. In some embodiments, the one or more additional peptide or polypeptide is fused at the C-terminus of the lasso peptidase or the lasso cyclase. In some embodiments, a polynucleotide encoding the fusion protein comprises a nucleic acid sequence encoding the lasso peptidase or the lasso cyclase, wherein the 5′ end of the nucleic acid sequence is linked to a nucleic acid sequence encoding the one or more additional peptide or polypeptide. In some embodiments, a polynucleotide encoding the fusion protein comprises a nucleic acid sequence encoding the lasso peptidase or the lasso cyclase, wherein the 3′ end of the nucleic acid sequence is linked to a nucleic acid sequence encoding the one or more additional peptide or polypeptide. In some embodiments, the fusion protein comprises an amino acid linker between the lasso peptidase or the lasso cyclase and the one or more additional peptide or polypeptide. In some embodiments, the fusion protein does not comprise an amino acid linker between the lasso peptidase or the lasso cyclase and the one or more additional peptide or polypeptide.

In some embodiments, the fusion protein comprised a lasso peptidase or a lasso cyclase fused to one or more additional peptide or polypeptide. In some embodiments, the more additional peptide or polypeptide comprises a peptide or polypeptide encoded by a lasso peptide gene cluster. Examples of peptide or polypeptide that can be fused with a lasso precursor peptide or a lasso core peptide according to the present disclosure include but are not limited to (i) a lasso precursor peptide; (ii) a lasso core peptide; (iii) a lasso peptidase; (iv) a lasso cyclase, (v) a RRE; or (vi) any combinations of (i) to (vi). In specific embodiments, the fusion protein comprises at least one lasso cyclase and at least one lasso peptidase. In specific embodiments, the fusion protein comprises at least one lasso cyclase fused to a RRE. In specific embodiments, the fusion protein comprises at least one lasso peptidase fused to a RRE.

In some embodiments, the fusion protein comprised a lasso peptidase or a lasso cyclase fused to one or more additional peptide or polypeptide. In some embodiments, the one or more additional peptide or polypeptide comprises a peptide or polypeptide that facilitates production of the lasso peptidase or lasso cyclase through cell-free biosynthesis. Examples of peptide or polypeptide that can be fused with the lasso peptidase or lasso cyclase according to the present disclosure include but are not limited to (i) a peptide or polypeptide that increases the level of transcription of the lasso peptidase or lasso cyclase in the CFB system; (ii) a peptide or polypeptide that increases the level of translation of the lasso peptidase or lasso cyclase in the CFB system; (iii) a peptide or polypeptide that improves stability of the lasso peptidase or lasso cyclase; (vi) a peptide or polypeptide that improves solubility of the lasso peptidase or lasso cyclase; (v) a peptide or polypeptide that enables or facilitates the detection of the lasso peptidase or lasso cyclase; (vi) a peptide or polypeptide that enables or facilitates purification of the lasso peptidase or lasso cyclase; (vii) a peptide or polypeptide that enables or facilitates immobilization of the lasso peptidase or lasso cyclase; or (viii) any combination of (i) to (vii).

In some embodiments, the fusion protein comprised a lasso peptidase or a lasso cyclase fused to one or more additional peptide or polypeptide. In some embodiments, the one or more additional peptide or polypeptide comprises a biologically active peptide or polypeptide. Examples of biologically active peptide or polypeptide that can be fused with a lasso peptidase or a lasso cyclase according to the present disclosure include but are not limited to (i) a peptide or polypeptide capable of modulating the reaction catalyzing activity of the lasso peptidase or lasso cyclase; (ii) a peptide or polypeptide capable of modulating target specificity of the lasso peptidase or lasso cyclase; (iii) an enzyme having the same or different enzymatic activity as the lasso peptidase or lasso cyclase; or any combination of (i) to (iii).

In some embodiments, the fusion protein comprised a RIPP recognition element (RRE) fused to one or more additional peptide or polypeptide. In some embodiments, the one or more additional peptide or polypeptide is fused to the N-terminus of the RRE. In some embodiments, the one or more additional peptide or polypeptide is fused at the C-terminus of the RRE. In some embodiments, a polynucleotide encoding the fusion protein comprises a nucleic acid sequence encoding the RRE, wherein the 5′ end of the nucleic acid sequence is linked to a nucleic acid sequence encoding the one or more additional peptide or polypeptide. In some embodiments, a polynucleotide encoding the fusion protein comprises a nucleic acid sequence encoding the RRE, wherein the 3′ end of the nucleic acid sequence is linked to a nucleic acid sequence encoding the one or more additional peptide or polypeptide. In some embodiments, the fusion protein comprises an amino acid linker between the RRE and the one or more additional peptide or polypeptide. In some embodiments, the fusion protein does not comprise an amino acid linker between RRE and the one or more additional peptide or polypeptide.

In some embodiments, the fusion protein comprised a RIPP recognition element (RRE) fused to one or more additional peptide or polypeptide. In some embodiments, the more additional peptide or polypeptide comprises a peptide or polypeptide encoded by a lasso peptide gene cluster. Examples of peptide or polypeptide that can be fused with a lasso precursor peptide or a lasso core peptide according to the present disclosure include but are not limited to (i) a lasso precursor peptide; (ii) a lasso core peptide; (iii) a lasso peptidase; (iv) a lasso cyclase, (v) a RRE; or (vi) any combinations of (i) to (vi). In specific embodiments, the fusion protein comprises at least one lasso precursor peptide fused to a RRE. In specific embodiments, the fusion protein comprises at least one lasso core peptide fused to a RRE. In specific embodiments, the fusion protein comprises at least one lasso cyclase fused to a RRE. In specific embodiments, the fusion protein comprises at least one lasso peptidase fused to a RRE.

In some embodiments, the fusion protein comprised a RIPP recognition element (RRE) fused to one or more additional peptide or polypeptide. In some embodiments, the one or more additional peptide or polypeptide comprises a peptide or polypeptide that facilitates production of the RRE through cell-free biosynthesis. Examples of peptide or polypeptide that can be fused with the RRE according to the present disclosure include but are not limited to (i) a peptide or polypeptide that increases the level of transcription of the RRE in the CFB system; (ii) a peptide or polypeptide that increases the level of translation of the RRE in the CFB system; (iii) a peptide or polypeptide that improves stability of the RRE; (vi) a peptide or polypeptide that improves solubility of the RRE; (v) a peptide or polypeptide that enables or facilitates the detection of the RRE; (vi) a peptide or polypeptide that enables or facilitates purification of the RRE; (vii) a peptide or polypeptide that enables or facilitates immobilization of the RRE; or (viii) any combination of (i) to (vii).

In some embodiments, the fusion protein comprised a RIPP recognition element (RRE) fused to one or more additional peptide or polypeptide. In some embodiments, the one or more additional peptide or polypeptide comprises a biologically active peptide or polypeptide. Examples of biologically active peptide or polypeptide that can be fused with a RRE according to the present disclosure include but are not limited to (i) a peptide or polypeptide capable of modulating the reaction catalyzing activity of the lasso peptidase or lasso cyclase; (ii) a peptide or polypeptide capable of modulating target specificity of the lasso peptidase or lasso cyclase; (iii) an enzyme having the same or different enzymatic activity as the lasso peptidase or lasso cyclase; or any combination of (i) to (iii).

In particular embodiments, the lasso precursor peptide genes are fused at the 5 ‘-terminus of the DNA template strand of the gene to oligonucleotide sequences that encode peptides or proteins, such as sequences encoding maltose-binding protein (MBP) or small ubiquitin-like modifier protein (SUMO), which enhance the stability, solubility, and production of the desired TX-TL products (Marblestone, J. G., et al., Protein Sci, 2006, 15, 182-189). In particular embodiments, the lasso precursor peptides are fused at the C-terminus of the leader sequences to form conjugates with peptides or proteins, such as maltose-binding protein or small ubiquitin-like modifier protein, which enhance the stability, solubility, and production of the fused MBP-lasso or SUMO-lasso precursor peptide.

In particular embodiments, the lasso precursor peptide genes or lasso core peptide genes are fused at the 3′-terminus of the DNA template strand of the gene to oligonucleotide sequences that encode peptides or proteins, such as sequences encoding maltose-binding protein (MBP) or small ubiquitin-like modifier protein (SUMO), which enhance the stability, solubility, and production of the desired TX-TL products. In particular embodiments, the lasso precursor peptides, lasso core peptides, or lasso peptides are fused at the N-terminus to form conjugates with peptides or proteins, such as maltose-binding protein or small ubiquitin-like modifier protein, which enhance the stability, solubility, and production of the fused MBP-lasso or SUMO-lasso precursor peptide.

In particular embodiments, the lasso precursor peptide genes or lasso core peptide genes are fused at the 5′-terminus of the DNA template strand of the gene to oligonucleotide sequences that encode a peptide or protein, with or without a linker, such as sequences encoding amino acid linkers connected to antibodies or antibody fragments, which provide bivalent lasso-antibody products that have enhanced activity against a single target cell or receptor or enhanced activity against two different target cells or receptors. In yet other embodiments, the lasso precursor peptides, lasso core peptides, or lasso peptides are fused at the C-terminus, with or without a linker, to form conjugates with peptides or proteins, such as amino acid linkers connected to antibodies or antibody fragments, which provide bivalent lasso-antibody products that have enhanced activity against a single target cell or receptor or enhanced activity against two different target cells or receptors.

In particular embodiments, the lasso precursor peptide genes or lasso core peptide genes are fused at the 5′-terminus of the DNA template strand of the gene to oligonucleotide sequences that encode peptides or proteins, with or without a linker, such as sequences encoding peptide tags for affinity purification or immobilization, including his-tags, a strep-tags, or FLAG-tags. In some embodiments, the lasso precursor peptides, lasso core peptides, or lasso peptides are fused at the C-terminus of the core peptides to form conjugates with other peptides or proteins, with or without a linker, such as peptide tags for affinity purification or immobilization, including his-tags, a strep-tags, or FLAG-tags.

In particular embodiments, lasso precursor peptides, lasso core peptides, or lasso peptides are fused to molecules that can enhance cell permeability or penetration into cells, for example through the use of arginine-rich cell-penetrating peptides such as TAT peptide, penetratin, and flock house virus (FHV) coat peptide (Brock, R, Bioconjug. Chem., 2014, 25, 863-868). In particular embodiments, a lasso precursor peptide gene or core peptide gene is fused at the 3′-terminus to oligonucleotide sequences that encode arginine-rich cell-penetrating peptides or proteins, including oligonucleotide sequences that encode penetratin, and flock house virus (FHV) coat peptide or similar peptides that contain guanidinium groups or a combination of lysine and guanidinium groups (Wender, P. A., et al., Adv. Drug Deliv. Rev., 2008, 60, 452-472). In particular embodiments, a lasso precursor peptide, lasso core peptide, or lasso peptide is fused at the C-terminus to peptides that promote cell penetration such as arginine-rich cell-penetrating peptides or proteins, including amino acid sequences that encode TAT peptide, penetratin, and flock house virus (FHV) coat peptide or similar peptides that contain guanidinium groups or a combination of lysine and guanidinium groups.

In particular embodiments, the lasso precursor peptide genes or lasso core peptide genes are fused at the 5′-terminus of the DNA template strand of the gene to oligonucleotide sequences that encode peptides or proteins, with or without a linker, such as sequences encoding peptide epitopes that are known to bind with high affinity to antibodies, cell surface proteins, or cell surface receptors, including cytokine binding epitopes, integrin ligand binding epitopes, and the like. In particular embodiments, the lasso precursor peptides, lasso core peptides, or lasso peptides are fused at the C-terminus to peptides or proteins, with or without a linker, such as peptide epitopes that are known to bind with high affinity to antibodies, cell surface proteins, or cell surface receptors, including cytokine binding epitopes, integrin ligand binding epitopes, and the like.

In particular embodiments, the cell-free biosynthesis reactions are conducted with a minimal set of lasso peptide biosynthesis components combined and contacted with genes that encode additional proteins or enzymes, including genes that encode RIPP recognition elements (RREs). In other embodiments, cell-free biosynthesis reactions are conducted with a minimal set of lasso peptide biosynthesis components combined with additional isolated proteins or enzymes, including RREs.

In particular embodiments, cell-free biosynthesis reactions are conducted with a minimal set of lasso peptide biosynthesis components combined and contacted with genes that encode additional proteins or enzymes, including genes that encode lasso peptide modifying enzymes such as N-methyltransferases, O-methyltransferases, biotin ligases, glycosyltransferases, esterases, acylases, acyltransferases, aminotransferases, amidases, hydroxylases, dehydrogenases, halogenases, kinases, RiPP heterocyclases, RiPP cyclodehydratases, and prenyltransfemses.

In particular embodiments, cell-free biosynthesis reactions are conducted with a minimal set of lasso peptide biosynthesis components combined and contacted with additional isolated proteins or enzymes, including lasso peptide modifying enzymes such as N-methyltransferases, O-methyltransferases, biotin ligases, glycosyltransferases, esterases, acylases, acyltransferases, aminotransferases, amidases, hydroxylases, dehydrogenases, halogenases, kinases, RiPP heterocyclases, RiPP cyclodehydratases, and prenyltransfemses.

In particular embodiments, cell-free biosynthesis methods described herein are used to produce lasso peptides and lasso peptide analogs by combining and contacting a minimal set of lasso peptide biosynthesis components, including, for example: (1) isolated precursor peptides or precursor peptide fusions, combined together and contacted with isolated proteins that include a lasso peptidase and a lasso cyclase, or fusions thereof, (2) oligonucleotides (linear or circular constructs of DNA or RNA) that encode for precursor peptides or precursor peptide fusions, combined together and contacted with isolated proteins that include a lasso peptidase and a lasso cyclase, or fusions thereof, (3) isolated precursor peptides or precursor peptide fusions, combined together and contacted with oligonucleotides that encode for a lasso peptidase and a lasso cyclase, or fusions thereof, (4) oligonucleotides that encode for lasso precursor peptides, a lasso peptidase, and a lasso cyclase, or fusions thereof, combined together and contacted, (5) isolated core lasso peptides combined and contacted with isolated lasso cyclases, or fusions thereof, (6) oligonucleotides that encode for core lasso peptides combined and contacted with isolated lasso cyclases, or fusions thereof, or (7) oligonucleotides that encode for core lasso peptides combined and contacted with oligonucleotides that encode for lasso cyclases, or fusions thereof, in a cell-free reaction mixture.

In particular embodiments, cell-free biosynthesis of lasso peptides is conducted with isolated peptide and enzyme components in standard buffered media, such as phosphate-buffered saline or tris-buffered saline, in each case containing salts, ATP, and co-factors facilitating enzyme activity. In some embodiments, cell-free biosynthesis of lasso peptides is conducted in a CFB reaction mixture using genes that require transcription (TX) and translation (TL) to afford the lasso precursor peptide and/or lasso peptide biosynthetic enzymes in situ, and such cell-free biosynthesis processes are conducted in cell extracts derived from prokaryotic or eukaryotic cells (Gagoski, D., et al., Biotechnol. Bioeng. 2016; 113: 292-300; Culler, S. et al., PCT Appl. No. WO2017/031399).

In some embodiments, lasso precursor peptides, lasso core peptides, lasso peptides, lasso peptide analogs, lasso peptidases, and/or lasso cyclases are fused to other peptides or proteins, with or without linkers between the partners, to enhance expression, to enhance solubility, to enhance cell permeability or penetration, to provide stability, to facilitate isolation and purification, and/or to add a distinct functionality. A variety of protein scaffolds may be used as fusion partners for lasso peptides, lasso peptide analogs, lasso core peptides, lasso precursor peptides, lasso peptidases, and/or lasso cyclases, including but not limited to maltose-binding protein (MBP), glutathione S-transferase (GST), thioredoxin (TRX), Nus A protein, ubiquitin (UB), and the small ubiquitin-like modifier protein SUMO (De Marco, V., et al., Biochem. Biophys. Res. Commun., 2004, 322, 766-771; Wang, C., et al., Biochem. J., 1999, 338, 77-81). In other embodiments, peptide fusion partners are used for rapid isolation and purification of lasso precursor peptides, lasso core peptides, lasso peptides, lasso peptide analogs, lasso peptidases, and/or lasso cyclases, including His6-tags, strep-tags, and FLAG-tags (Pryor, K. D., Leiting, B., Protein Expr. Purif., 1997, 10, 309-319; Einhauer A. Jungbauer A., J. Biochem. Biophys. Methods, 2001, 49, 455-465; Schmidt, T. G., Skerra, A., Nature Protocols, 2007, 2, 1528-1535). In other embodiments, lasso peptides, lasso core peptides, or lasso precursor peptides are fused to molecules that can enhance cell permeability or pentration into cells, for example through the use of arginine-rich cell-penetrating peptides such as TAT peptide, penetratin, and flock house virus (FHV) coat peptide (Brock, R, Bioconjug. Chem., 2014, 25, 863-868; Herce, H. D., et al., J. Am. Chem. Soc., 2014, 136, 17459-17467; Ter-Avetisyan, G. et al., J. Biol. Chem., 2009, 284, 3370-3378; Schmidt, N., et al., FEBS Lett., 2010, 584, 1806-1813; Tunnemann, G. et al., FASEB J., 2006, 20, 1775-1784; Lattig-Tunnemann, G. et al., Nat. Commun., 2011, 2, 453, DOI: 10.1038/ncomms1459; Reissmann, S., J Pept Sci., 2014, 20, 760-784).

In other embodiments, peptide or protein fusion partners are used to introduce new functionality into lasso core peptides, lasso peptides or lasso peptide analogs, such as the ability to bind to a separate biological target, e.g., to form a bispecific molecule for multitarget engagement. In such cases, a variety of peptide or protein partners may be fused with lasso core peptides, lasso peptides or lasso peptide analogs, with or without linkers between the partners, including but not limited to peptide binding epitopes, cytokines, antibodies, monoclonal antibodies, single domain antibodies, antibody fragments, nanobodies, monobodies, affibodies, nanofitins, fluorescent proteins (e.g., GFP), avimers, fibronectins, designed ankyrins, lipocallans, cyclotides, conotoxins, or a second lasso peptide with the same or different binding specificity, e.g., to form bivalent or bispecific lasso peptides (Huet, S., et al., PLoS One, 2015, 10 (11): e0142304., doi:10.1371/journal.pone.0142304; Steeland, S., et al., Drug Discov. Today, 2016, 21, 1076-1113; Lipovsek, D., Prot. Eng., Des. Sel., 2011, 24, 3-9; Sha, F., et al., Prot. Sci., 2017, 26, 910-924; Silverman, J., et al., Nat. Biotech., 2005, 23, 1556-1561; Pluckthun, A., Diagnostics, and Therapy, Annu. Rev. Pharmacol. Toxicol., 2015, 55, 489-511; Nelson, A. L., mAbs, 2010, 2, 77-83; Boldicke, T., Prot. Sci, 2017, 26, 925-945; Liu, Y., et al., ACS Chem Biol., 2016, 11, 2991-2995; Liu, T., et al., Proc. Nat. Acad. Sci. USA., 2015, 112, 1356-1361; Müller D., Pharmacol Ther., 2015, 154, 57-66; Weidmann, J.; Craik, D. J., J. Experimental Bot., 2016, 67, 4801-4812; Burman, R., et al., J. Nat. Prod. 2014, 77, 724-736; Reinwarth, M., et al., Molecules, 2012, 17, 12533-12552; Uray, K., Hudecz, F., Amino Acids, Pept. Prot., 2014, 39, 68-113).

In other embodiments, a lasso precursor peptide gene is fused at the 3′-terminus of the leader sequence, or at the 5′-terminus of the core peptide sequence of the DNA template strand of the gene, to oligonucleotide sequences that encode peptides or proteins, including sequences that encode maltose-binding protein (MBP) or small ubiquitin-like modifier protein (SUMO), which enhance the stability and/or production of the desired products formed using a TX-TL-based CFB method or process (Marblestone, J. G., et al., Protein Sci, 2006, 15, 182-189). In some embodiments, the lasso precursor peptides are fused at the N-terminus of the leader sequence or at the C-terminus of the core sequence to form conjugates with peptides or proteins, including maltose-binding protein or small ubiquitin-like modifier protein, which enhance the stability and/or production of the lasso peptide precursor fusion product, e.g., MBP-lasso precursor peptide or SUMO-lasso precursor peptide. In yet other embodiments, a lasso core peptide gene is fused at at the 5′-terminus of the core peptide sequence of the DNA template strand of the gene to oligonucleotide sequences that encode peptides or proteins, including sequences that encode maltose-binding protein (MBP) or small ubiquitin-like modifier protein (SUMO), which enhance the stability and/or production of the desired products formed using a TX-TL-based CFB method or process. In alternative embodiments, a lasso core peptide is fused at the C-terminus of the core sequence to form conjugates with peptides or proteins, including maltose-binding protein or small ubiquitin-like modifier protein, which enhance the stability and/or production of the lasso peptide precursor fusion product, e.g., MBP-lasso core peptide or SUMO-lasso core peptide. In alternative embodiments, a lasso peptide is fused at the N-terminus or at the C-terminus of the lasso peptide to form conjugates with peptides or proteins, including maltose-binding protein or small ubiquitin-like modifier protein, which enhance the stability and/or production of the lasso peptide precursor fusion product, e.g., MBP-lasso peptide or SUMO-lasso peptide.

In other embodiments, lasso peptidase or lasso cyclase genes are fused at the 5′- or 3′-terminus with oligonucleotide sequences that encode peptides or proteins, including sequences that encode maltose-binding protein (MBP) or small ubiquitin-like modifier protein (SUMO). In alternative embodiments, lasso peptidases or lasso cyclases are fused at the N-terminus or the C-terminus to peptides or proteins, such as maltose-binding protein (MBP) or small ubiquitin-like modifier protein (SUMO), which enhance the stability and/or production of the desired TX-TL products.

In other embodiments, a lasso precursor peptide gene or core peptide gene is fused at the 5′-terminus of the DNA template strand of the gene to oligonucleotide sequences that encode arginine-rich cell-penetrating peptides or proteins, including oligonucleotide sequences that encode penetratin, and flock house virus (FHV) coat peptide or similar peptides that contain guanidinium groups or a combination of lysine and guanidinium groups (Wender, P. A., et al., Adv. Drug Deliv. Rev., 2008, 60, 452-472). In other embodiments, a lasso precursor peptide, lasso core peptide, or lasso peptide is fused at the C-terminus to peptides that promote cell penetration such as arginine-rich cell-penetrating peptides or proteins, including amino acid sequences that encode TAT peptide, penetratin, and flock house virus (FHV) coat peptide or similar peptides that contain guanidinium groups or a combination of lysine and guanidinium groups.

In alternative embodiments, the lasso precursor peptide genes or lasso core peptide genes are fused at the 5′-terminus of the DNA template strand of the gene to oligonucleotide sequences that encode a peptide or protein, with or without a linker, such as sequences encoding amino acid linkers connected to antibodies or antibody fragments, which provide bivalent lasso-antibody products that exhibit enhanced activity against an individual biological target, receptor, or cell type, or enhanced activity against two different biological targets, receptors, or cell types. In some embodiments, the lasso precursor peptides or lasso core peptides or lasso peptides are fused at the C-terminus to form conjugates with peptides or proteins, such as amino acid linkers connected to antibodies or antibody fragments, which provide bivalent lasso-antibody products that exhibit enhanced activity against an individual biological target, receptor, or cell type, or enhanced activity against two different biological targets, receptors, or cell types.

In alternative embodiments, the lasso precursor peptide genes or lasso core peptide genes are fused at the 5′-terminus of the DNA template strand of the gene to oligonucleotide sequences that encode a peptide or protein, with or without a linker, such as sequences encoding peptide tags for affinity purification or immobilization, including His-tags, strep-tags, or FLAG-tags. In some embodiments, the lasso precursor peptides or lasso core peptides or lasso peptides are fused at the C-terminus to form conjugates with peptides or proteins, such as, such as sequences that encode peptide tags for affinity purification or immobilization, including His-tags, strep-tags, or FLAG-tags.

In some embodiments, the lasso precursor peptide genes or lasso core peptide genes are fused at the 5′-terminus of the DNA template strand of the gene to oligonucleotide sequences that encode peptides or proteins, with or without a linker, such as sequences encoding peptide epitopes that are known to bind with high affinity to antibodies, cell surface proteins, or cell surface receptors, including cytokine binding epitopes, integrin ligand binding epitopes, and the like. In some embodiments, the lasso precursor peptides, lasso core peptides, or lasso peptides are fused at the C-terminus to peptides or proteins, with or without a linker, such as peptide epitopes that are known to bind with high affinity to antibodies, cell surface proteins, or cell surface receptors, including cytokine binding epitopes, integrin ligand binding epitopes, and the like.

In other embodiments, cell-free biosynthesis reactions are conducted with a minimal set of lasso peptide biosynthesis components combined with genes that encode additional peptides, proteins or enzymes, including genes that encode RIPP recognition elements (RREs) or oligonucleotides that encode RREs that are fused to the 5′ or 3′ end of a lasso precursor peptide gene, a lasso core peptide gene, a lasso peptidase gene or a lasso cyclase gene. In other embodiments, cell-free biosynthesis reactions are conducted with a minimal set of lasso peptide biosynthesis components, including lasso precursor peptides, lasso peptidases, or lasso cyclase that are fused to RREs at the N-terminus or C-terminus. In other embodiments, cell-free biosynthesis reactions are conducted with a minimal set of lasso peptide biosynthesis components combined and contacted with additional isolated proteins or enzymes, including (RREs).

In some embodiments, cell-free biosynthesis reactions are conducted with a minimal set of lasso peptide biosynthesis components combined with genes that encode additional proteins or enzymes, including genes that encode lasso peptide modifying enzymes such as N-methyltransferases, O-methyltonsferases, biotin ligases, glycosyltransferases, esterases, acylases, acyltransferases, aminotransferases, amidases, halogenases, kinases, RiPP heterocyclases, RiPP cyclodehydratases, and prenyltransferases.

In some embodiments, cell-free biosynthesis reactions are conducted with a minimal set of lasso peptide biosynthesis components combined and contacted with additional isolated proteins or enzymes, including lasso peptide modifying enzymes such as N-methyltransferases, O-methyltransferases, biotin ligases, glycosyltransferases, esterases, acylases, acyltransferases, aminotransferases, amidases, halogenases, kinases, RiPP heterocyclases, RiPP cyclodehydratases, and prenyltransferases.

In some embodiments, cell-free biosynthesis of lasso peptides is conducted with isolated peptide and enzyme components in standard buffered media, such as phosphate-buffered saline or tris-buffered saline, in each case containing salts, ATP, and co-factors for lasso peptidase and lasso cyclase enzymatic activity. In some embodiments, cell-free biosynthesis of lasso peptides is conducted using genes that require transcription (TX) and translation (TL) to afford the lasso precursor peptide and/or lasso peptide biosynthetic enzymes in situ, and such in vitro biosynthesis processes are conducted in cell extracts derived from prokaryotic or eukaryotic cells (Gagoski, D., et al., Biotechnol. Bioeng. 2016; 113: 292-300; Culler, S. et al., PCT Appl. No. W2017/031399).

Particularly, in some embodiments, the CFB system further comprises co-factors for one or more enzymes to perform the enzymatic function. In some embodiments, the CFB system comprises co-factors of the lasso peptidase. In some embodiments, the CFB system comprises co-factors of the lasso cyclase. In some embodiments, the CFB system further comprises ATP. In some embodiments, the CFB system further comprises salts. In some embodiments, the CFB system components are contained in a buffer media. In some embodiments, the CFB system components are contained in phosphate-buffered saline solution. In some embodiments, the CFB system components are contained in a tris-buffered saline solution.

In some embodiments, the CFB system comprises the biosynthetic and metabolic machinery of a cell, without using a living cell. In some embodiments, the CFB system comprises a CFB reaction mixture as provided herein. In some embodiments, the CFB system comprises a cell extract as provided. In some embodiments, the cell extract is derived from prokaryotic cells. In some embodiments, the cell extract is derived from eukaryotic cells. In some embodiments, the CFB system comprises a supplemented cell extract provided herein. In some embodiments, the CFB system comprises in vitro transcription and translation machinery as provided herein.

In some embodiments, the CFB system comprises cell extract from one type of cell. In some embodiments, the CFB system comprises cell extracts from two or more types of cells. In some embodiments, the CFB system comprises cell extracts of 2, 3, 4, 5 or more than 5 types of cells. In some embodiments, the different types of cells are from the same species. In other embodiments, the different types of cells are from different species. In particular embodiments, the CFB system comprises cell extract from one or more types of cell, species, or class of organism, such as E. coli and/or Saccharomyces cerevisiae, and/or Streptomyces lividans. In some embodiments, the CFB system comprises cell extracts from yeast. In some embodiments, the CFB system comprises cell extracts from both E. coli and yeast.

Cell extract from cells that natively produce a lasso peptide can offer a robust transcription/translation machinery, and/or cellular context that facilitates proper protein folding or activity, or supply precursors for the lasso peptide pathway. Accordingly, in some embodiments, the CFB system comprises cell extract from a chassis organism cells, mixed with one or a combination of two or more cell extracts derived from different species. In particular embodiments, the CFB system comprises cell extract from E. coli cells, mixed with cell extracts from one or more organism that natively produces lasso peptide. In particular embodiments, the CFB system comprises cell extract from E. coli cells, mixed with cell extracts from one or more organism that relates to an organism that natively produces lasso peptide. In alternative embodiments, CFB system comprises cell extract from a chassis organism cells supplemented with one or more purified or isolated factors known to facilitate lasso peptide production from an organism that natively produces a lasso peptide.

In some embodiments, the CFB systems including in vitro transcription/translation (TX-TL) systems, provided herein to produce lasso peptides and lasso peptide analogs comprises whole cell, cytoplasmic or nuclear extract from a single organism. In some embodiments, the CFB systems comprise whole cell, cytoplasmic or nuclear extract from E. coli. In some embodiments, the CFB systems comprise whole cell, cytoplasmic or nuclear extract from Saccharomyces cerevisiae (S. cerevisiae). In some embodiments, the CFB systems comprise whole cell, cytoplasmic or nuclear extract from an organism of the Actinomyces genus, e.g., a Streptomyces. In some embodiments, the CFB systems including in vitro transcription/translation (TX-TL) systems, provided herein to produce lasso peptides and lasso peptide analogs comprises mixtures of whole cell, cytoplasmic, and/or nuclear extracts from the same or different organisms, such as one or more species selected from E. coli, S. cerevisiae, or the Actinomyces genus.

In some embodiments, strain engineering approaches as well as modification of the growth conditions are used (on the organism from which an at least one extract is derived) towards the creation of cell extracts as provided herein, to generate mixed cell extracts with varying proteomic and metabolic capabilities in the final CFB reaction mixture. In alternative embodiments, both approaches are used to tailor or design a final CFB reaction mixture for the purpose of synthesizing and characterizing lasso peptides, or for the creation of lasso peptide analogs through combinatorial biosynthesis approaches.

In some embodiments, the CFB system provided herein comprises whole cell, cytoplasmic or nuclear extracts from a bacterial cell or eukaryotic cell, including insect, plant, fungal, yeast, or mammalian cells. In alternative embodiments, the CFB system provided herein comprises whole cell, cytoplasmic or nuclear extracts from a bacterial cell or eukaryotic cell, including insect, plant, fungal, yeast, or mammalian cells, and are designed, produced and processed in away to maximize efficacy and yield in the production of desired lasso peptides or lasso peptide analogs.

In some embodiment, the CFB system comprises cell extract from at least two different bacterial cells. In some embodiment, the CFB system comprises cell extract from at least two different fungal cells. In some embodiment, the CFB system comprises cell extract from at least two different yeast cells. In some embodiment, the CFB system comprises cell extract from at least two different insect cells. In some embodiment, the CFB system comprises cell extract from at least two different plant cells. In some embodiment, the CFB system comprises cell extract from at least two different mammalian cells. In some embodiment, the CFB system comprises cell extract from at least two different species selected from bacteria, fungus, yeast, insect, plant, and mammal. In particular embodiments, the CFB system comprises cell extract derived from an Escherichia or a Escherichia coli (E. coli). In particular embodiments, the CFB system comprises cell extract derived from a Streptomyces or an Actinobacteria. In particular embodiments, the CFB system comprises cell extract derived from an Ascomycota, Basidiomycota or a Saccharomycetales. In particular embodiments, the CFB system comprises cell extract derived from a Penicillium or a Trichocomaceae. In particular embodiments, the CFB system comprises cell extract derived from a Spodoptera, a Spodoptera frugiperda, a Trichoplusia or a Trichoplusia ni. In particular embodiments, the CFB system comprises cell extract derived from a Poaceae, a Triticum, or a wheat germ. In particular embodiments, the CFB system comprises cell extract derived from a rabbit reticulocyte. In particular embodiments, the CFB system comprises cell extract derived from a HeLa cell.

In alternative embodiments, the CFB system comprises cell extract derived from any prokaryotic and eukaryotic organism including, but not limited to, bacteria, including Archaea, eubacteria, and eukaryotes, including yeast, plant, insect, animal, and mammal, including human cells. In alternative embodiments, at least one of the cell extracts used in the CFB system provided herein comprises an extract derived from: Escherichia coli, Saccharomyces cerevisiae, Saccharomyces kluyveri, Candida boidinii, Clostridium kluyveri, Clostridium acetobutylicum, Clostridium beyerinckii, Clostridium saccharoperbutylacetonicum, Clostridium pefringens, Clostridium dificile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandn, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermo-anaerobacter brockii, Metallosphaera sedula, Leuconostoc mesenteroides, Chlorofexus aurantiacus, Roseiexus castenholzii, Erythrobacter, Simmondsia chinensis, Acinetobacter species, including Acinetobacter calcoaceticus and Acinetobacter baylyi, Porphyromonas gingivalis, Sulfolobus tokodai, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2155, Mycobacterium avium subsp. paratuberculosis K-10, Mycobacterium marinum M, Tsukamurella paurometabola DSM20162, Cyanobium PCC7001, Dictyostelium discoideum AX4.

In alternative embodiments, at least one cell, cytoplasmic or nuclear extract used in the CFB system provided herein comprises a cell extract from or comprises an extract derived from: Acinetobacter baumannii Naval-82, Acinetobacter sp. ADPI, Acinetobacter sp. strain M-1, Actinobacillus succinogenes 130Z, Allochromatium vinosum DSM180, Amycolatopsis methanolica, Arabidopsis thaliana, Atopobium parvulum DSM20469, Azotobacter vinelandii DJ, Bacillus alcalophilus ATCC 27647, Bacillus azotoformans IMG 9581, Bacillus coagulans 36D1, Bacillus megaterium, Bacillus methanolicus MGA3, Bacillus methanolicus PB1, Bacillus methanolicus PB-1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DM 9485, Chlorofexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM13275, Clostridium hylemonae DSM15053, Clostridium kluyveri, Clostridium kluyveri DSM555, Clostridium jungdahli, Clostridium ljungdahlii DSM13528, Clostridium methylpentosum DSM5476, Clostridium pasteurianum, Clostridium pasteurianum DSM525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desuftobacterium hafniense, Desulfotobacterium metallireducens DSM15288, Desulfotomaculum reducens M-1, Desulfovibrio africanus str. Walvis Bay, Desulfovibrio fructosovorans JJ, Desulfovibrio vulgaris str. Hildenborough, Desulfovibrio vulgaris sr. Miazaki F, Dictyostelium discoideum AX4, Escherichia coli, Escherichia coli K-12, Escherichia coli K-12MG1655, Eubacterium hallii DSM3353, Flavobacterium frigoris, Fusobacterium nucleatum subsp. polymorphum ATCC 10953, Geobacillus sp. Y4.1MC1, Geobacillus themodenitrifcans NG80-2, Geobacter bemidjiensis Bem, Geobacter sulfurreducens, Geobacter sulfurreducens PCA, Geobacillus stearothermophilus DSM2334, Haemophilus influenzae, Helicobacter pylori, Homo sapiens, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF303099, Metallosphaera sedula, Methanosarcina acetivorans, Methanosarcina acetivorans C2A, Methanosarcina barkeri, Methanosarcina mazei Tuc01, Methylobacter marinus, Methylobacterium extorquens, Methylobacterium extorquens AM1, Methylococcus capsulatas, Methylomonas aminofaciens, Moorella thermoacetica, Mycobacter sp. strain JCI DSM3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2155, Mycobacterium tuberculosis, Nitrosopumilus salaria BD31, Nirososphaera gargensis Ga9.2, Nocardia farcinica IFM10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrifcans, Penicillium chrysogenum, Photobacterium profundum 3TCK, Phytofermentans ISDg Pichia pastoris, Picrophilus torridus DSM9790, Porphyromonas gingivalis, Porphyromonas gingivalis W83, Pseudomonas aeruginosa PA01, Pseudomonas denitrifcans, Pseudomonas knackmussii, Pseudomonas putida, Pseudomonas sp, Pseudomonas syringae pv. syringae B728a, Pyrobaculum islandicum DSM4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxdans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, or Zea mays.

In alternative embodiments, CFB system provided herein comprises cell extract supplemented with additional ingredients, compositions, compounds, reagents, ions, trace metals, salts, elements, buffers and/or solutions. In alternative embodiments, the CFB system provided herein uses or fabricates environmental conditions to optimize the rate of formation or yield of a lasso peptide or lasso peptide analog.

In alternative embodiments, CFB system provided herein comprises a reaction mixture or cell extracts that are supplemented with a carbon source and other nutrients. In some embodiments, the CFB system can comprise any carbohydrate source, including but not limited to sugars or other carbohydrate substances such as glucose, xylose, maltose, arabinose, galactose, mannose, maltodextin, fuctose, sucrose and/or starch.

In alternative embodiments, CFB system provided herein comprises cell extract supplemented with all twenty proteinogenic naturally occurring amino acids and corresponding transfer ribionucleic acids (tRNAs). In alternative embodiments, CFB system provided herein comprises cell extract supplemented with adenosine triphosphate (ATP), and/or adenosine diphosphate (ADP). In alternative embodiments, CFB system provided herein comprises cell extract supplemented with glucose, xylose, maltose, arabinose, galactose, mannose, maltodextrin, fructose, sucrose and/or starch. In alternative embodiments, CFB system provided herein comprises cell extract supplemented with purine and guanidine nucleotides, adenosine triphosphate, guanosine triphosphate, cytosine triphosphate, and uridine triphosphate. In alternative embodiments, CFB system provided herein comprises cell extract supplemented with cyclic-adenosine monophosphate (cAMP) and/or 3-phosphoglyceric acid (3-PGA). In alternative embodiments, CFB system provided herein comprises cell extract supplemented with nicotimamide adenine dinucleotides NADH and/or NAD, or nicotimamide adenine dinucleotide phosphates, NADPH, and/or NADP, or combinations thereof. In alternative embodiments, CFB system provided herein comprises cell extract supplemented with amino acid salts such as magnesium glutamate and/or potassium glutamate. In alternative embodiments, CFB system provided herein comprises cell extract supplemented with buffering agents such as HEPES, TRIS, spermidine, or phosphate salts. In alternative embodiments, CFB system provided herein comprises cell extract supplemented with salts, including but not limited to, potassium phosphate, sodium chloride, magnesium phosphate, and magnesium sulfate. In alternative embodiments, CFB system provided herein comprises cell extract supplemented with folinic acid and co-enzyme A (CoA). In alternative embodiments, CFB system provided herein comprises cell extract supplemented with crowding agents such as PEG 8000, Ficoll 70, or Ficoll 400, or combinations thereof. For a general description of cell-free extract production and preparation, see: Krinsky, N., et al., PLoS ONE, 2016, 11(10): e0165137.

In alternative embodiments, the CFB system is maintained under aerobic or substantially aerobic conditions. In some embodiments, the aerobic or substantially aerobic conditions can be achieved, for example, by sparging with air or oxygen, shaking under an atmosphere of air or oxygen, stirring under an atmosphere of air or oxygen, or combinations thereof. In alternative embodiments, the CFB system is maintained is maintained under anaerobic or substantially anaerobic conditions. In some embodiments, the anaerobic or substantially anaerobic conditions can be achieved, for example, by first sparging the medium with nitrogen and then sealing the wells or reaction containers, or by shaking or stirring under a nitrogen atmosphere. Briefly, anaerobic conditions refer to an environment devoid of oxygen. In some embodiments, substantially anaerobic conditions include, for example, CFM processes conducted such that the dissolved oxygen concentration in the medium remains between 0 and 10% of saturation. In some embodiments, substantially anaerobic conditions also include performing the CFB methods and processes inside a sealed chamber maintained with an atmosphere of less than 1% oxygen. The percent of oxygen can be maintained by, for example, sparging the CFB reaction with an N₂/CO₂ mixture or other suitable non-oxygen gas or gases.

In some embodiments, the CFB system is maintained at a desirable pH for high rates and yields in the production of lasso peptides and lasso peptide analogs. In some embodiments, the CFB system is maintained at neutral pH. In some embodiments, the CFB system is maintained at a pH of around 7 by addition of a buffer. In some embodiments, the CFB system is maintained at a pH of around 7 by addition of base, such as NaOH. In some embodiments, the CFB system is maintained at a pH of around 7 by addition of an acid.

In alternative embodiments, the CFB system comprises cell extract supplemented with one or more enzymes of the central metabolism pathways of a microorganism. In alternative embodiments, the CFB system comprises cell extract supplemented with one or more nucleic acids that encode one or more enzymes of the central metabolism pathway of a microorganism. In some embodiments, the central metabolism pathway enzyme is selected from enzymes of the tricarboxylic acid cycle (TCA, or Krebs cycle), the glycolysis pathway or the Citric Acid Cycle, or enzymes that promote the production of amino acids.

In some embodiments, the preparation CFB reaction mixtures and cell extracts employed for the CFB system as provided herein comprises characterization of the CFB reaction mixtures and cell extracts using proteomic approaches to assess and quantify the proteome available for the production of lasso peptides and lasso peptide analogs. In alternative embodiments, ¹³C metabolic flux analysis (MFA) and/or metabolomics studies are conducted on CFB reaction mixtures and cell extracts to create a flux map and characterize the resulting metabolome of the CFB reaction mixture and cell extract or extracts.

In some embodiments, the CFB systems provided herein comprise one or more nucleic acid that (i) encodes one or more lasso precursor peptide; (ii) encodes one or more lasso core peptide; (iii) encodes one or more lasso peptide synthesizing enzyme; (iv) encodes one or more lasso peptidase; (v) encodes one or more lasso cylase; (vi) encodes one or more RRE; (vii) forms or encodes one or more components of the in vitro TX-TL machinery; (viii) form or encodes one or more lasso peptide biosynthetic pathway operon; (ix) form one or more biosynthetic gene cluster; (x) form one or more lasso peptide gene cluster; (xi) encodes one or more additional enzymes; (xii) encodes one or more enzyme co-factors; or (xiii) any combination of (i) to (xii). In some embodiments, the nucleic acid that encodes or forms any combination of (i) to (xii) is a single nucleic acid molecule.

In some embodiments, the nucleic acid molecule comprises one or more sequences selected from the odd numbers of SEQ ID Nos: 1-2630, or a sequence having at least 30% identity thereto. In some embodiments, the nucleic acid molecule comprises at least one sequences selected from the odd numbers of SEQ ID Nos: 1-2630, or a sequence having at least 30% identity thereto, and at least one sequence encoding a lasso peptidase as described herein. In some embodiments, the nucleic acid molecule comprises at least one sequences selected from the odd numbers of SEQ ID Nos: 1-2630 or a sequence encoding a lasso cyclase as described herein. In some embodiments, the nucleic acid molecule comprises at least one sequences selected the odd numbers of SEQ ID Nos: 1-2630 or a sequence having at least 30% identity thereto, and at least one sequence encoding a lasso RRE as described herein. In some embodiments, the nucleic acid molecule comprises at least one sequences selected from the odd numbers of SEQ ID Nos: 1-2630, or a sequence having at least 30% identity thereto, at least one sequence encoding a lasso peptidase as described herein, and at least one sequence encoding a lasso cyclase as described herein. In some embodiments, the nucleic acid molecule comprises at least one sequences selected from the odd numbers of SEQ ID Nos: 1-2630 or a sequence having at least 30% identity thereto, at least one sequence encoding a lasso peptidase as described herein, and at least one sequence encoding a lasso RRE as described herein. In some embodiments, the nucleic acid molecule comprises at least one sequences selected from the odd numbers of SEQ ID Nos: 1-2630 or a sequence having at least 30% identity thereto, at least one sequence encoding a lasso cyclase as described herein, and at least one sequence encoding a lasso RRE as described herein. In some embodiments, the nucleic acid molecule comprises at least one sequences selected from the odd numbers of SEQ ID Nos: 1-2630 or a sequence having at least 30% identity thereto, at least one sequence encoding a lasso peptidase as described herein, and at least one sequence encoding a lasso cyclase as described herein, and at least one sequence encoding a lasso RRE as described herein. In some embodiments, the nucleic acid molecule comprises one or more combination of nucleic acid sequences listed in Table 2.

In some embodiments, the CFB system comprises one or more nucleic acids encoding for a peptide or polypeptide having a sequence selected from the even number of SEQ ID Nos: 1-2630 or a sequence having at least 30% identity thereto. In some embodiments, the CFB system comprises one or more nucleic acids encoding for a peptide or polypeptide having a sequence selected from peptide Nos: 1316-2336 or a natural sequence having at least 30% identity thereto. In some embodiments, the CFB system comprises one or more nucleic acids encoding for a peptide or polypeptide having a sequence selected from peptide Nos: 2337-3761 or a natural sequence having at least 30% identity thereto. In some embodiments, the CFB system comprises one or more nucleic acids encoding for a peptide or polypeptide having a sequence selected from peptide Nos: 37624593 or a natural sequence having at least 30% identity thereto. In some embodiments, the CFB system comprises at least one nucleic acid encoding for a peptide or polypeptide having a sequence selected from the even number of SEQ ID Nos: 1-2630 or a sequence having at least 30% identity thereto, and at least one nucleic acid encoding for a peptide or polypeptide having a sequence selected from peptide Nos: 1316-2336 or a natural sequence having at least 30% identity thereto. In some embodiments, the CFB system comprises at least one nucleic acid encoding for a peptide or polypeptide having a sequence selected from peptide Nos: 1316-2336 or a natural sequence having at least 30% identity thereto, and at least one nucleic acid encoding for a peptide or polypeptide having a sequence selected from peptide Nos: 2337-3761 or a natural sequence having at least 30% identity thereto. In some embodiments, the CFB system comprises at least one nucleic acid encoding for a peptide or polypeptide having a sequence selected from the even number of SEQ ID Nos: 1-2630 or a sequence having at least 30% identity thereto, and at least one nucleic acid encoding for a peptide or polypeptide having a sequence selected from peptide Nos: 2337-3761 or a natural sequence having at least 30% identity thereto. In some embodiments, the CFB system comprises at least one nucleic acid encoding for a peptide or polypeptide having a sequence selected from the even number of SEQ ID Nos: 1-2630 or a sequence having at least 30% identity thereto, and at least one nucleic acid encoding for a peptide or polypeptide having a sequence selected from peptide Nos: 3762-4593 or a natural sequence having at least 30% identity thereto. In some embodiments, the CFB system comprises at least one nucleic acid encoding for a peptide or polypeptide having a sequence selected from peptide Nos: 1316-2336 or a natural sequence having at least 30% identity thereto, and at least one nucleic acid encoding for a peptide or polypeptide having a sequence selected from peptide Nos: 37624593 or a natural sequence having at least 30% identity thereto. In some embodiments, the CFB system comprises at least one nucleic acid encoding for a peptide or polypeptide having a sequence selected from peptide Nos: 2337-3761 or a natural sequence having at least 30% identity thereto, and at least one nucleic acid encoding for a peptide or polypeptide having a sequence selected from peptide Nos: 37624593 or a natural sequence having at least 30% identity thereto. In some embodiments, the CFB system comprises at least one nucleic acid encoding for a peptide or polypeptide having a sequence selected from the even number of SEQ ID Nos: 1-2630 or a sequence having at least 30% identity thereto, at least one nucleic acid encoding for a peptide or polypeptide having a sequence selected from peptide Nos: 1316-2336 or a natural sequence having at least 30% identity thereto, and at least one nucleic acid encoding for a peptide or polypeptide having a sequence selected from peptide Nos: 2337-3761 or a natural sequence having at least 30% identity thereto. In some embodiments, the CFB system comprises at least one nucleic acid encoding for a peptide or polypeptide having a sequence selected from the even number of SEQ ID Nos: 1-2630 or a sequence having at least 30% identity thereto, at least one nucleic acid encoding for a peptide or polypeptide having a sequence selected from peptide Nos: 1316-2336 or a natural sequence having at least 30% identity thereto, and at least one nucleic acid encoding for a peptide or polypeptide having a sequence selected from peptide Nos: 37624593 or a natural sequence having at least 30% identity thereto. In some embodiments, the CFB system comprises at least one nucleic acid encoding for a peptide or polypeptide having a sequence selected from peptide Nos: 1316-2336 or a natural sequence having at least 30% identity thereto, at least one nucleic acid encoding for a peptide or polypeptide having a sequence selected from peptide Nos: 2337-3761 or a natural sequence having at least 30% identity thereto, and at least one nucleic acid encoding for a peptide or polypeptide having a sequence selected from peptide Nos: 37624593 or a natural sequence having at least 30% identity thereto. In some embodiments, the CFB system comprises at least one nucleic acid encoding for a peptide or polypeptide having a sequence selected from the even number of SEQ ID Nos: 1-2630 or a sequence having at least 30% identity thereto, at least one nucleic acid encoding for a peptide or polypeptide having a sequence selected from peptide Nos: 1316-2336 or a natural sequence having at least 30% identity thereto, at least one nucleic acid encoding for a peptide or polypeptide having a sequence selected from peptide Nos: 2337-3761 or a natural sequence having at least 30% identity thereto, and at least one nucleic acid encoding for a peptide or polypeptide having a sequence selected from peptide Nos: 3762-4593 or a natural sequence having at least 30% identity thereto. In some embodiments, the nucleic acid molecules encode one or more combination of peptides or polypeptides listed in Table 2.

In some embodiment, a variant of a peptide or of a polypeptide has an amino acid sequence having at least about 30% identity to the peptide or polypeptide. In some embodiment, a homolog of a peptide of a polypeptide has an amino acid sequence having at least about 40% identity to the peptide or polypeptide. In some embodiment, a homolog of a peptide of a polypeptide has an amino acid sequence having at least about 50% identity to the peptide or polypeptide. In some embodiment, a homolog of a peptide of a polypeptide has an amino acid sequence having at least about 60% identity to the peptide or polypeptide. In some embodiment, a homolog of a peptide of a polypeptide has an amino acid sequence having at least about 70% identity to the peptide or polypeptide. In some embodiment, a homolog of a peptide of a polypeptide has an amino acid sequence having at least about 80% identity to the peptide or polypeptide. In some embodiment, a homolog of a peptide of a polypeptide has an amino acid sequence having at least about 90% identity to the peptide or polypeptide. In some embodiment, a homolog of a peptide of a polypeptide has an amino acid sequence having at least about 95% identity to the peptide or polypeptide. In some embodiment, a homolog of a peptide of a polypeptide has an amino acid sequence having at least about 97% identity to the peptide or polypeptide. In some embodiment, a homolog of a peptide of a polypeptide has an amino acid sequence having at least about 98% identity to the peptide or polypeptide. As described herein a peptidic variant includes natural or non-natural variant of the lasso precursor peptide and/or lasso core peptide. As described herein a peptidic variant include natural variant of the lasso peptidase, lasso cyclase and/or RRE.

In some embodiments, the nucleic acids are isolated or substantially isolated before added into the CFB system. In some embodiments, the nucleic acids are endogenous to a cell extract forming the CFB system. In some embodiments, the nucleic acids are synthesized in vitro. In alternative embodiments, the nucleic acids are in a linear or a circular form. In some embodiments, the nucleic acids are contained in a circular or a linearized plasmid, vector or phage DNA. In alternative embodiments, the nucleic acids comprise enzyme coding sequences operably linked to a homologous or a heterologous transcriptional regulatory sequence, optionally a transcriptional regulatory sequence is a promoter, an enhancer, or a terminator of transcription. In alternative embodiments, the substantially isolated or synthetic nucleic acids comprise at least about 50, 100, 200, 250, 300, 350, 400, 450, 500, 550, 600 or more base pair ends upstream of the promoter and/or downstream of the terminator.

In alternative embodiments, the CFB system provided herein comprises one or more nucleic acid sequences in the form of expression constructs, vehicles or vectors. In alternative embodiments, nucleic acids used in the CFB system provided herein are operably linked to an expression (e.g., transcription or translational) control sequence, e.g., a promoter or enhancer, e.g., a control sequence functional in a cell from which an extract has been derived. In alternative embodiments, the CFB system comprises one or more nucleic acid molecules in the forms of expression constructs, expression vehicles or vectors, plasmids, phage vectors, viral vectors or recombinant viruses, episomes and artificial chromosomes, including vectors and selection sequences or markers containing nucleic acids. In alternative embodiments, the expression vectors also include one or more selectable marker genes and appropriate expression control sequences.

In some embodiments, selectable marker genes also can be included, for example, on plasmids that contain genes for lasso peptide synthesis to provide resistance to antibiotics or toxins, to complement auxotrophic deficiencies, or to supply critical nutrients not in an extract. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vehicle (e.g., a vector or plasmid) or in separate expression vehicles. For single vehicle/vector expression, the encoding nucleic acids can be operationally linked to one common expression control sequence or linked to different expression control sequences, such as one inducible promoter and one constitutive promoter.

In alternative embodiments, nucleic acid analysis such as Northern blots or polymerase chain reaction (PCR) amplification of mRNA, or immunoblotting, are used for analysis of expression of gene products, e.g., enzyme-encoding message; any analytical method can be used to test the expression of an introduced nucleic acid sequence or its corresponding gene product. The exogenous nucleic acid can be expressed in a sufficient amount to produce the desired product, and expression levels can be optimized to obtain sufficient expression.

In alternative embodiments, multiple enzyme-encoding nucleic acids (e.g., two or more genes) are fabricated on one polycistronic nucleic acid. In alternative embodiments, one or more enzyme-coding nucleic acids of a desired lasso peptide synthetic pathway are fabricated on one linear or circular DNA. In alternative embodiments, all or a subset of the enzyme-encoding nucleic acid of an enzyme-encoding lasso peptide synthesizing operon or biosynthetic gene cluster are contained on separate linear nucleic acids (separate nucleic acid strands), optionally in equimolar concentrations in a whole cell, cytoplasmic or nuclear extract, as described above, and optionally, each separate linear nucleic acid comprises 1, 2, 3, 4, 5, 6, 7, 8, 9, or 10 or more genes or enzyme-encoding sequences, and optionally the linear nucleic acid is present in a cell extract at a concentration of about 10 nM (nanomolar), 15 nM, 20 nM, 25 nM, 30 nM, 35 nM, 40 nM, 45 nM or 50 nM or more or between about 1 nM and 100 nM.

5.5 Optimization and Diversifying of Lasso Peptides

In one aspect, provided herein are CFB systems and related methods for optimizing lasso peptides or lasso peptide analogs for desirable properties and functionality.

Chemical or Enzymatic Modification

In some embodiments, the CFB systems comprises one or more components function to modify the lasso peptide or lasso peptide analog produced by the CFB system. In some embodiments, the lasso peptides or lasso peptide analogs produced by the CFB systems or methods are chemically modified. In some embodiments, the lasso peptides or lasso peptide analogs produced by the CFB systems or methods are enzymatically modified.

In particular embodiments, the core peptides or the lasso peptides produced by cell-free biosynthesis are modified further through chemical steps. In some embodiments, the core peptides or the lasso peptides produced by cell-free biosynthesis are modified through chemical steps that allow the attachment of chemical linker units connected to small molecules to the C-terminus of the core peptide or the lasso peptide. In some embodiments, the core peptides or the lasso peptides produced by cell-free biosynthesis are modified through the attachment of chemical linkers connected to small molecules to the side chain of functionalized amino acids (e.g., the OH or serine, threonine, or tyrosine, or the N of lysine). In other embodiments, the lasso core peptides or the lasso peptides produced by cell-free biosynthesis are modified further through chemical steps. In other embodiments, the lasso core peptides or the lasso peptides produced by cell-free biosynthesis are modified by PEGylation. In other embodiments, the lasso core peptides or the lasso peptides produced by cell-free biosynthesis are modified by biotinylation. In other embodiments, the lasso core peptides or the lasso peptides produced by cell-free biosynthesis are modified through the formation of esters, sulfonyl esters, phosphonate esters, or amides by reaction with the side chain of functionalized amino acids (e.g., the OH or serine, threonine, or tyrosine, or the N of lysine). In yet other embodiments, the core peptides or the lasso peptides produced by cell-free biosynthesis may contain non-natural amino acids which are modified further through chemical steps. In yet other embodiments, the core peptides or the lasso peptides produced by cell-free biosynthesis may contain non-natural amino acids which are modified through the use of click chemistry involving amino acids with azide or alkyne functionality within the side chains (Presolski, S. I., et al., Curr Protoc Chem Biol., 2011, 3, 153-162). In yet other embodiments, the core peptides or the lasso peptides produced by cell-free biosynthesis may contain non-natural amino acids which are modified further through metathesis chemistry involving alkene or alkyne groups within the amino acid side chains (Cromm, P. M., et al., Nat. Comm., 2016, 7, 11300; Gleeson, E. C., et al., Tetrahedron Lett., 2016, 57, 4325-4333).

In particular embodiments, the lasso peptide or lasso peptide analogs generated by a CFB method or system are modified chemically or by enzyme modification. Exemplary modifications to the lasso peptide or lasso peptide analogs include but are not limited to halogenation, lipidation, pegylation, glycosylation, adding hydrophobic groups, myristoylation, palmitoylation, isoprenylation, prenylation, lipoylation, adding a flavin moiety (optionally comprising addition of: a flavin adenine dinucleotide (FAD) an FADH₂, a flavin mononucleotide (FMN), an FMNH₂), phospho-pantetheinylation, heme C addition, phosphorylation, acylation, alkylation, butyrylation, carboxylation, malonylation, hydroxylation, adding a halide group, iodination, propionylation, S-glutathionylation, succinylation, glycation, adenylation, thiolation, condensation (optionally the “condensation” comprising addition of: an amino acid to an amino acid, an amino acid to a fatty acid, an amino acid to a sugar), or a combination thereof, and optionally the enzyme modification comprises modification of the lasso peptide by one or more enzymes comprising: a CoA ligase, a phosphorylase, a kinase, a glycosyl-transferase, a halogenase, a methyltransferase, a hydroxylase, a lambda phage GamS enzyme (optionally used with a bacterial or an E. coli extract, optionally at a concentration of about 3.5 mM), a Dsb (disulfide bond) family enzyme (optionally DsbA), or a combination thereof, or optionally the enzymes comprise one or more central metabolism enzyme (optionally tricarboxylic acid cycle (TCA, or Krebs cycle) enzymes, glycolysis enzymes or Pentose Phosphate Pathway enzymes), and optionally the chemical or enzyme modification comprises addition, deletion or replacement of a substituent or functional groups, optionally a hydroxyl group, an amino group, a halogen, an alkyl or a cycloalkyl group, optionally by hydration, biotinylation, hydrogenation, an aldol condensation reaction, condensation polymerization, halogenation, oxidation, dehydrogenation, or creating one or more double bonds.

In some embodiments, cell-free biosynthesis is used to facilitate the creation of mutational variants of lasso peptides using the above method. For example, in some embodiments, the synthesis of codon mutants of the core lasso peptide gene sequence which are used in the cell-free biosynthesis process, thus enabling the creation of high density lasso peptide diversity libraries. In some embodiments, cell-free biosynthesis is used to facilitate the creation of large mutational lasso peptide libraries using, for example, using site-saturation mutagenesis and recombination methods or in vitro display technologies (Josephson, K., et al., Drug Discov. Today, 2014, 19, 388-399; Doi, N., et al., PLoS ONE, 2012, 7, e30084, pp 1-8; Josephson, K., et al., J. Am. Chem. Soc., 2005, 127, 11727-11735; Kretz, K. A., et al, Methods Enzymol., 2004, 388, 3-11; Nannemann, D. P, et al., Future Med Chem., 2011, 3, 809-819).

In some embodiments, cell-free biosynthesis methods are used to facilitate the creation of mutational variants of lasso peptides by introducing non-natural amino acids into the core peptide sequence, through either biological or chemical means, followed by formation of the lasso structure using the cell-free biosynthesis methods involving, at minimum, a lasso cyclase gene or a lasso cyclase for lasso peptide production as described above.

Optimization Via Directed Evolution, Mutagenesis or Display Libraries

As disclosed herein, a set of nucleic acids encoding the desired activities of a lasso peptide biosynthesis pathway can be introduced into a host organism to produce a lasso peptide, or can be introduced into a cell-free biosynthesis reaction mixture containing a cell extract or other suitable medium to produce a lasso peptide. In some cases, it can be desirable to modify the properties or biological activities of a lasso peptide to improve its therapeutic potential. In other cases, it can be desirable to modify the activity or specificity of lasso peptide biosynthesis pathway enzymes or proteins to improve the production of lasso peptides. For example, mutations can be introduced into an encoding nucleic acid molecule (e.g., a gene), which ultimately leads to a change in the amino acid sequence of a protein, enzyme, or peptide, and such mutated proteins, enzymes, or peptides can be screened for improved properties. Such optimization methods can be applied, for example, to increase or improve the activity or substrate scope of an enzyme, protein, or peptide and/or to decrease an inhibitory activity. Lasso peptides are derived from precursor peptides that are ribsomally produces by transcription and translation of a gene. Ribosomally produced peptides, such as lasso precursor peptides, are known to be readily evolved and optimized through variation of nucleotide sequences within genes that encode for the amino acid residues that comprise the peptide. Large libraries of peptide mutational variants have been produced by methods well known in the art, and some of these methods are referred to as directed evolution.

Directed evolution is a powerful approach that involves the introduction of mutations targeted to a specific gene or an oligonucleotide sequence containing a gene in order to improve and/or alter the properties or production of an enzyme, protein or peptide (e.g., a lasso peptide). Improved and/or altered enzymes, proteins or peptides can be identified through the development and implementation of sensitive high-throughput assays that allow automated screening of many enzyme or peptide variants (for example, >10⁴). Iterative rounds of mutagenesis and screening typically are performed to afford an enzyme or peptide with optimized properties. Computational algorithms that can help to identify areas of the gene for mutagenesis also have been developed and can significantly reduce the number of enzyme or peptide variants that need to be generated and screened (See: Fox, R J., et al., Trends Biotechnol., 2008, 26, 132-138; Fox, R J., et al., Nature Biotechnol., 2007, 25, 338-344). Numerous directed evolution technologies have been developed and shown to be effective at creating diverse variant libraries, and these methods have been successfully applied to the improvement of a wide range of properties across many enzyme and protein classes (for reviews, see: Hibbert et al., Biomol. Eng., 2005,22, 11-19; Huisman and Lalonde, In Biocatalysis in the pharmaceutical and biotechnology industries, pgs. 717-742 (2007), Patel (ed.), CRC Press; Otten and Quax, Biomol. Eng., 2005, 22, 1-9; and Sen et al., Appl. Biochem. Biotechnol., 2007, 143, 212-223). Enzyme and protein characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening of ligand or substrate binding to include non-natural substrates; inhibition (Kᵢ), to remove inhibition by products, substrates, or key intermediates; activity (k_{cat}), to increase enzymatic reaction rates to achieve desired flux; isoelectric point (pI) to improve protein or peptide solubility; acid dissociation (pKₐ) to vary the ionization state of the protein or peptide with repect to pH; expression levels, to increase protein or peptide yields and overall pathway flux; oxygen stability, for operation of air-sensitive enzymes or peptides under aerobic conditions; and anaerobic activity, for operation of an aerobic enzyme or peptide in the absence of oxygen.

A number of exemplary methods have been developed for the mutagenesis and diversification of genes and oligonucleotides to intorduce desired properties into specific enzymes, proteins and peptides. Such methods are well known to those skilled in the art. Any of these can be used to alter and/or optimize the activity of a lasso peptide biosynthetic pathway enzyme, protein, or peptide, including a lasso precursor peptide, a lasso core peptide, or a lasso peptide. Such methods include, but are not limited to error-prone polymerase chain reaction (EpPCR), which introduces random point mutations by reducing the fidelity of DNA polymerase in PCR reactions (See: Pritchard et al., J Theor. Biol., 2005, 234:497-509); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res., 2004, 32:e145; and Fujii et al., Nat. Protoc., 2006, 1, 2493-2497); DNA, Gene, or Family Shuffling, which typically involves digestion of two or more variant genes with nucleases such as Dnase I or EndoV to generate a pool of random fragments that are reassembled by cycles of annealing and extension in the presence of DNA polymerase to create a library of chimeric genes (Stemmer, Proc. Nat. Acad. Sci. USA., 1994, 91, 10747-10751; and Stemmer, Nature, 1994, 370, 389-391); Staggered Extension (StEP), which entails template priming followed by repeated cycles of 2-step PCR with denaturation and very short duration of annealing/extension (as short as 5 sec) (Zhao et al., Nat. Biotechnol., 1998, 16, 258-261); Random Priming Recombination (RPR), in which random sequence primers are used to generate many short DNA fragments complementary to different segments of the template (Shao et al., Nucleic Acids Res., 1998, 26, 681-683).

Additional methods include Heteroduplex Recombination, in which linearized plasmid DNA is used to form heteroduplexes that are repaired by mismatch repair (See: Volkov et al, Nucleic Acids Res., 1999, 27:e18; Volkov et al., Methods Enzymol., 2000, 328, 456463); Random Chimeragenesis on Transient Templates (RACHITI), which employs Dnase I fragmentation and size fractionation of single-stranded DNA (ssDNA) (See: Coco et al., Nat. Biotechnol., 2001, 19, 354-359); Recombined Extension on Truncated Templates (RETT), which entails template switching of unidirectionally growing strands from primers in the presence of unidirectional ssDNA fragments used as a pool of templates (See: Lee et al., J Mol. Cat., 2003, 26, 119-129); Degenerate Oligonucleotide Gene Shuffling (DOGS), in which degenerate primers are used to control recombination between molecules; (Bergquist and Gibbs, Methods Mol. Biol., 2007, 352, 191-204; Bergquist et al., Biomol. Eng., 2005, 22, 63-72; Gibbs et al., Gene, 2001, 271, 13-20); Incremental Truncation for the Creation of Hybrid Enzymes (ITCHY), which creates a combinatorial library with 1 base pair deletions of a gene or gene fragment of interest (See: Ostermeier et al., Proc. Nat. Acad Sci. USA., 1999, 96, 3562-3567; and Ostermeier et al., Nat. Biotechnol., 1999, 17, 1205-1209); Thio-Incremental Truncation for the Creation of Hybrid Enzymes (THIO-ITCHY), which is similar to ITCHY except that phosphothioate dNTPs are used to generate truncations (See: Lutz et al., Nucleic Acids Res., 2001, 29, E16); SCRATCHY, which combines two methods for recombining genes, ITCHY and DNA Shuffling (See: Lutz et al., Proc. Nat. Acad. Sci. USA., 2001, 98, 11248-11253); Random Drift Mutagenesis (RNDM), in which mutations made via epPCR are followed by screening/selection for those retaining usable activity (See: Bergquist et al., Biomol. Eng., 2005, 22, 63-72); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (See: Wong et al., Biotechnol. J., 2008, 3, 74-82; Wong et al., Nucleic Acids Res., 2004, 32, e26; Wong et al., Anal. Biochem., 2005, 341, 187-189); Synthetic Shuffling, which uses overlapping oligonucleotides designed to encode “all genetic diversity in targets” and allows a very high diversity for the shuffled progeny (See: Ness et al., Nat. Biotechnol., 2002, 20, 1251-1255); Nucleotide Exchange and Excision Technology NexT, which exploits a combination of dUTP incorporation followed by treatment with uracil DNA glycosylase and then piperidine to perform endpoint DNA fragmentation (See: Muller et al., Nucleic Acids Res., 33:e117).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (See: Sieber et al., Nat. Biotechnol., 2001, 19, 456460); Gene Site Saturation Mutagenesis™ (GSSM™), in which the starting materials include a supercoiled double stranded DNA (dsDNA) plasmid containing an insert and two primers which are degenerate at the desired site of mutations, enabling all amino acid variations to be introduced individually at each position of a protein or peptide (See: Kretz et al., Methods Enzymol., 2004, 388, 3-11); Combinatorial Cassette Mutagenesis (CCM), which involves the use of short oligonucleotide cassettes to replace limited regions with a large number of possible amino acid sequence alterations (See: Reidhaar-Olson et al. Methods Enzymol., 1991, 208, 564-586; Reidhaar-Olson et al. Science, 1988, 241, 53-57); Combinatorial Multiple Cassette Mutagenesis (CMCM), which is essentially similar to CCM and uses epPCR at high mutation rate to identify hot spots and hot regions and then extension by CMCM to cover a defined region of protein sequence space (See: Reetz et al., Angew. Chem. Int. Ed Engl., 2001, 40, 3589-3591); the Mutator Strains technique, in which conditional ts mutator plasmids, utilizing the mutD5 gene, which encodes a mutant subunit of DNA polymerase III, to allow increases of 20 to 4000× in random and natural mutation frequency during selection and block accumulation of deleterious mutations when selection is not required (See: Selifonova et al., Appl. Environ. Microbiol., 2001, 67, 3645-3649); Low et al., J Mol. Biol., 1996, 260, 3659-3680).

Additional exemplary methods include Look-Through Mutagenesis (LTM), which is a multidimensional mutagenesis method that assesses and optimizes combinatorial mutations of a selected set of amino acids (See: Rajpal et al, Proc. Natl. Acad Sci. USA., 2005, 102, 8466-8471); Gene Reassembly, which is a homology-independent DNA shuffling method that can be applied to multiple genes at one time or to create a large library of chimeras (multiple mutations) of a single gene (See: Short, J. M., U.S. Pat. No. 5,965,408, Tunable GeneReassembly™); in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (See: Hayes et al., Proc. Natl. Acad. Sci. USA., 2002, 99, 15926-15931); and Iterative Saturation Mutagenesis (ISM), which involves using knowledge of structure/function to choose a likely site for enzyme improvement, performing saturation mutagenesis at chosen site using a mutagenesis method such as Stratagene QuikChange (Stratagene; San Diego Calif.), screening/selecting for desired properties, and, using improved clone(s), starting over at another site and continue repeating until a desired activity is achieved (See: Reetz et al., Nat. Protoc., 2007, 2, 891-903; Reetz et al., Angew. Chem. Int. Ed Engl., 2006, 45, 7745-7751).

In some embodiments, the systems and libraries disclosed herein may be used in connection with a display technology, such that the components in the present systems and/or libraries may be conveniently screened for a property of interest. Various display technologies are known in the art, for example, involving the use of microbial organism to present a substance of interest (e.g., a lasso peptide or lasso peptide analog) on their cell surface. Such display technology may be used in connection with the present disclosure.

Furthermore, a rapid way to create large libranes of diverse peptides involves the use of display technologies (For a review, see: Ullman, C. G., et al., Briefings Functional Genomics, 2011, 10, 125-134). Peptide display technologies offer the benefit that specific peptide encoding information (e.g., RNA or DNA sequence information) is linked to, or otherwise associated with, each corresponding peptide in a library, and this information is accessible and readable (e.g., by amplifying and sequencing the attached DNA oligonucleotide) after a screening event, thus enabling identification of the individual peptides within a large library that exhibit desirable properties (e.g., high binding affinity). The cell-free biosynthesis methods provided herein can facilitate and enable the creation of large lasso peptide libraries containing lasso peptide analogs that can be screened for favorable properties. Lasso peptide mutants that exhibit the desired improved properties (hits) may be subjected to additional rounds of mutagenesis to allow creation of highly optimized lasso peptide variants. The CFB methods and systems described herein for the production of lasso peptides and lasso peptide analogs, used in combination with peptide display technologies, establishes a platform to rapidly produce high density libraries of lasso peptide variants and to identify promising lasso peptide analogs with desirable properties.

In addition to biological methods for the evolution of lasso peptides, also can be conducted using chemical synthesis methods. For example, large combinatorial peptide libraries (e.g., >10⁶ members) containing mutational variants can be synthesized by using known solution phase or solid phase peptide synthesis technologies (See review. Shin, D.-S., et al., J Biochem. Mol. Bio., 2005, 38, 517-525). Chemical peptide synthesis methods can be used to produce lasso precursor peptide variants, or alternatively, lasso core peptide variants, containing a wide range of alpha-amino acids, including the natural proteinogenic amino acids, as well as non-natural and/or non-proteinogenic amino acids, such as amino acids with non-proteinogenic side chains, or alternatively D-amino acids, or alternatively beta-amino acids. Cyclization of these chemically synthesized lasso precursor peptides or lasso core peptides can provide vast lasso peptide diversity that incorporates stereochemical and functional properties not seen in natural lasso peptides.

Any of the aforementioned methods for lasso peptide mutagenesis and/or display can be used alone or in any combination to improve the performance of lasso peptide biosynthesis pathway enzymes, proteins, and peptides. Similarly, any of the aforementioned methods for mutagenesis and/or display can be used alone or in any combination to enable the creation of lasso peptide variants which may be selected for improved properties.

In one embodiment of the invention, a mutational library of lasso peptide precursor peptides is created and converted by a lasso peptidase and a lasso cyclase into a library of lasso peptide variants that are screened for improved properties. In another embodiment, a mutational library of lasso core peptides is created and converted by a lasso cyclase into a library of lasso peptide variants that are screened for improved properties.

In other embodiments of the invention, a mutational library of lasso peptidases is created and screened for improved properties, such as increased temperature stability, tolerance to a broader pH range, improved activity, improved activity without requiring an RRE, broader lasso precursor peptide substrate scope, improved tolerance and rate of conversion of lasso precursor peptide mutational variants, improved tolerance and rate of conversion of lasso precursor peptide N-terminal or C-terminal fusions, improved yield of lasso peptides and lasso peptide analogs, and/or lower product inhibition. In other embodiments of the invention, a mutational library of lasso cyclases is created and screened for improved properties, such as increased temperature stability, tolerance to a broader pH range, improved activity when used in combination with a lasso peptidase to convert a lasso precursor peptide, improved activity on a core peptide lacking a leader peptide, broader lasso precursor peptide substrate scope, broader lasso core peptide substrate scope, improved tolerance and rate of conversion of lasso core peptide mutational variants, improved tolerance and rate of conversion of lasso core peptide C-terminal fusions, improved yield of lasso peptides and lasso peptide analogs, and/or lower product inhibition.

5.6 Methods of Producing Lasso Peptides and Lasso Peptide Libraries

Provided herein are various uses of the present CFB system. In certain aspects, disclosed herein are methods for producing a lasso peptide or lasso peptide analog using the CFB system. In some embodiments, the method for producing a lasso peptide comprises (a) providing a CFB system comprising a minimal set of lasso peptide biosynthesis components; and (b) incubating the CFB system under a suitable condition to produce the lasso peptide. In some embodiments, the minimal set of lasso peptide biosynthesis components comprises one or more components functions to provide a lasso precursor peptide, and one or more components function to process the lasso precursor peptide into the lasso peptide. In some embodiments, the one or more components function to process the lasso precursor peptide into the lasso peptide comprises one or more selected from a lasso peptidase, a lasso cyclase and a RRE. In some embodiments, the one or more components function to process the lasso precursor peptide into the lasso peptide consist of a lasso peptidase and a lasso cyclase.

In some embodiments, the method for producing a lasso peptide comprises (a) providing a CFB system comprising a minimal set of lasso peptide biosynthesis components; and (b) incubating the CFB system under a suitable condition to produce the lasso peptide. In some embodiments, the minimal set of lasso peptide biosynthesis components comprises one or more components functions to provide a lasso core peptide, and one or more components function to process the lasso core peptide into the lasso peptide. In some embodiments, the one or more components function to process the lasso core peptide into the lasso peptide comprises one or more selected from a lasso peptidase, a lasso cyclase and a RRE. In some embodiments, the one or more components function to process the lasso core into the lasso peptide consist of a lasso cyclase.

In some embodiments, the method for producing a lasso peptide analog comprises (a) providing a CFB system comprising a minimal set of lasso peptide biosynthesis components; and (b) incubating the CFB system under a suitable condition to produce the lasso peptide analog. In some embodiments, the minimal set of lasso peptide biosynthesis components comprises one or more components functions to provide a lasso precursor peptide, and one or more components function to process the lasso precursor into the lasso peptide analog. In some embodiments, the lasso precursor peptide comprises a lasso core peptide sequence that is mutated as compared to a wild-type sequence. In various embodiments, such mutation can be one or more amino acid substitution, deletion or addition. In some embodiments, the lasso precursor peptide comprises a lasso core peptide sequence that comprises at least one non-natural amino acid. In some embodiments, the one or more components function to process the lasso precursor peptide into the lasso peptide analog comprises an enzyme or chemical entity capable of modifying the lasso precursor peptide sequence or lasso peptide sequence. In various embodiments, such modification can be any chemical or enzymatic modifications described herein.

In particular embodiments, CFB methods and systems, provided herein for the synthesis of lasso peptides and lasso peptide analogs from a minimal set of lasso peptide biosynthetic pathway components, including processes for in vitro, or cell free, transcription/translation (TX-TL), comprise: (a) providing a CFB reaction mixture, including cell extracts or cell-free reaction media, as described or provided herein; (b) incubating the CFB reaction mixture with substantially isolated or synthetic nucleic acids encoding: a lasso precursor peptide; a lasso core peptide; a lasso peptide synthesizing enzyme or enzymes; a lasso peptide biosynthetic gene cluster, a lasso peptide biosynthetic pathway operon. In other embodiments, optionally provided is, a lasso peptide biosynthetic gene cluster comprising coding sequences for all or substantially all or a minimum set of enzymes for the synthesis of a lasso peptide or lasso peptide analog; a plurality of enzyme-encoding nucleic acids; a plurality of enzyme-encoding nucleic acids for at least two, several or all of the steps in the synthesis of a lasso peptide or lasso peptide analog; and optionally where the substantially isolated or synthetic nucleic acids comprise: (i) a gene or an oligonucleotide from a source other than the cell used for the cell extract (an exogenous nucleic acid), or an exogenous nucleic acid, gene, or oligonucleotide that has been engineered or mutated, optionally engineered or mutated in a protein coding region or in a non-coding region, (ii) a gene or an oligonucleotide from a cell used for the cell extract (an endogenous nucleic acid), or an endogenous nucleic acid that has been engineered or mutated, optionally engineered or mutated in a protein coding region or in a non-coding region, (iii) a gene or an oligonucleotide from one, both or several of the organisms used as a source for the cell extract, or, (iv) any or all of (i) to (iii).

In certain aspects, disclosed herein are methods for producing a lasso peptide library using the CFB system, the lasso peptide library comprising a plurality of species of lasso peptides and/or lasso peptide analogs, herein referred to as “lasso species.” In various embodiments, the plurality of lasso species in the library may have the same amino acid sequence or different amino acid sequences based on the process the library is generated. For example, in some embodiments, a plurality of lasso species in the library have the same amino acid sequences, while having different chemical or enzymatic modifications to the amino acid residues or side chains in the sequence. In some embodiments, a plurality of lasso species in the library have different amino acid sequences. In some embodiments, the plurality of lasso species in the library may be mixed together. In other embodiments, the plurality of lasso species in the library may be enclosed separately. In some embodiments, the plurality of lasso species forming the library may be individual purified. In other embodiments, the plurality of lasso species forming the library may be mixed with one or more components from the CFB system.

Various process may be used for generating a lasso peptide library using the CFB system. For example, to generate a lasso peptide library having a plurality of lasso species having different amino acid sequences, in some embodiments, the method comprises (a) providing a CFB system comprising a minimal set of lasso peptide biosynthesis components; and (b) incubating the CFB system under a suitable condition to produce the lasso peptide library; wherein the minimal set of lasso peptide biosynthesis components comprises (i) one or more polynucleotide encoding for a plurality of species of lasso precursor peptides and/or lasso core peptides, (ii) one or more components function to process the lasso precursor peptide and/or lasso core peptide into a plurality of lasso species. In some embodiments, the method further comprises separating the plurality of lasso species from one another.

In another exemplary embodiments, to generate a lasso peptide library having a plurality of lasso species having different amino acid sequences, in some embodiments, the method comprises (a) providing a CFB system comprising a minimal set of lasso peptide biosynthesis components; and (b) incubating the CFB system under a suitable condition to produce the lasso peptide library; wherein the minimal set of lasso peptide biosynthesis components comprises (i) one or more components function to provide a single species of lasso precursor peptide or lasso core peptide; and (ii) one or more components function to provide a plurality of species of lasso peptidases. In some embodiments, the plurality of species of lasso peptidases are capable of processing the lasso precursor peptide or lasso core peptide into a plurality of species of lasso peptides or lasso peptide analogs. In particular embodiments, the plurality of species of lasso peptidase are capable of cleaving the lasso precursor peptide at different locations to release a plurality of species of lasso core peptides.

In another exemplary embodiments, to generate a lasso peptide library having a plurality of lasso species having different conformations, in some embodiments, the method comprises (a) providing a CFB system comprising a minimal set of lasso peptide biosynthesis components; and (b) incubating the CFB system under a suitable condition to produce the lasso peptide library; wherein the minimal set of lasso peptide biosynthesis components comprises (i) one or more components function to provide a single species of lasso precursor peptide or lasso core peptide; and (ii) one or more components function to provide a plurality of species of lasso cyclase. In some embodiments, the plurality of species of lasso cyclase are capable of processing the lasso precursor peptide or lasso core peptide into a plurality of lasso species. In particular embodiments, the plurality of species of lasso cyclase are capable of linking the N-terminus of the lasso core peptide to a side chain of an amino acid residue located at different positions within the core peptide.

In another exemplary embodiments, to generate a lasso peptide library having a plurality of lasso species having both different amino acid sequences and conformations, in some embodiments, the method comprises (a) providing a CFB system comprising a minimal set of lasso peptide biosynthesis components; and (b) incubating the CFB system under a suitable condition to produce the lasso peptide library; wherein the minimal set of lasso peptide biosynthesis components comprises (i) one or more components function to provide a single species of lasso precursor peptide or lasso core peptide; (ii) one or more components function to provide a plurality of species of lasso peptidase; and (iii) one or more components function to provide a plurality of species of lasso cyclase. In some embodiments, the plurality of species of lasso peptidase and lasso cyclase are capable of processing the lasso precursor peptide or lasso core peptide into a plurality of lasso species. In particular embodiments, the plurality of species of lasso peptidase are capable of cleaving the lasso precursor peptide at different locations to release a plurality of species of lasso core peptides, and/or the plurality of species of lasso cyclase are capable of linking the N-terminus of the lasso core peptide to a side chain of an amino acid residue located at different positions within the core peptide.

In another exemplary embodiments, to generate a lasso peptide library having a plurality of lasso species having the same amino acid sequences with different amino acid modifications, the method comprises (a) providing a CFB system comprising a minimal set of lasso peptide biosynthesis components; and (b) incubating the CFB system under a suitable condition to produce the lasso peptide library; wherein the minimal set of lasso peptide biosynthesis components comprises (i) one or more polynucleotide encoding for a single species of a lasso precursor peptide or lasso core peptide, (ii) one or more components function to process the lasso precursor peptide or lasso core peptide into a single species of lasso peptide; (iii) one or more components function to modify the lasso peptide into a plurality of species having different amino acid modifications. In some embodiments, the method further comprises incubating the CFB system under a first condition suitable for generating a first species, and incubating the CFB system under a second condition suitable for generating a second species. In some embodiments, the method further comprises incubating the CFB system under a third or more conditions for generating a third or more species. In some embodiments, to generate species having diversified modifications, the method further comprises sequentially supplementing the CFB system with multiple components, each capable of generating a different species. In some embodiments, the method further comprises separating the species from one another.

In yet exemplary embodiments, to generate a lasso peptide library comprising lasso species having both diversified amino acid sequences and diversified amino acid modifications, the method comprises (a) providing a CFB system comprising a minimal set of lasso peptide biosynthesis components; and (b) incubating the CFB system under a suitable condition to produce the lasso peptide library; wherein the minimal set of lasso peptide biosynthesis components comprises (i) one or more components function to provide a plurality of species of lasso precursor peptides or lasso core peptides, (ii) one or more components function to process the lasso precursor peptide or lasso core peptide into a plurality of lasso species; and (iii) one or more components function to further diversify the lasso species into a plurality of species having different amino acid modifications.

In some embodiments, methods for generating a lasso peptide library comprises (a) providing a CFB system comprising a minimal set of lasso peptide biosynthesis components; and (b) incubating the CFB system under a suitable condition to produce the lasso peptide library; wherein the CFB system comprises (i) one or more components function to provide at least one lasso precursor peptides or lasso core peptides; (ii) one or more components function to provide a plurality of species of lasso peptidase; (ii) one or more components function to provide a plurality of species of lasso cyclase; (iv) one or more components function to further diversify the lasso species generated in the CFB system into a plurality of species having different amino acid modifications.

In some embodiments of the method for generating the library, the amino acid modifications are selected from the chemical modifications and enzymatic modifications described herein. In some embodiments, the polynucleotides encoding for a lasso precursor peptides or lasso core peptides is identified using a genomic mining algorithm as described herein. In some embodiments, the polynucleotides encoding for a lasso precursor peptides or lasso core peptides is identified using a mutagenesis method as described herein.

In some embodiments, cell-free biosynthesis systems are used to facilitate the discovery of new lasso peptides from Nature using the above methods involving, for example, the identification of lasso peptide biosynthesis genes using bioinformatic genome-mining algorithms followed by cloning or synthesis of pathway genes which are used in the cell-free biosynthesis process, thus enabling the rapid generation of new lasso peptide diversity libraries.

In some embodiments, cell-free biosynthesis systems are used to facilitate the creation of mutational variants of lasso peptides using methods involving, for example, the synthesis of codon mutants of the lasso precursor peptide or lasso core peptide gene sequence. Lasso precursor peptide or lasso core peptide gene or oligonucleotide mutants can be used in a cell-free biosynthesis process, thus enabling the creation of high density lasso peptide diversity libraries. In some embodiments, cell-free biosynthesis is used to facilitate the creation of large mutational lasso peptide libraries using, for example, site-saturation mutagenesis and recombination methods, or in vitro display technologies such as, for example, phage display, RNA display or DNA display (See: Josephson, K., et al., Drug Discov. Today, 2014, 19, 388-399; Doi, N., et al., PLoS ONE, 2012, 7, e30084, pp 1-8; Josephson, K., et al., J Am. Chem. Soc., 2005, 127, 11727-11735; Odegrip, R., et al., Proc. Nat. Acad Sci. USA., 2004, 101, 2806-2810; Gamkrelidze, M., Dabrowska, K., Arch Microbiol, 2014, 196, 473-479; Kretz, K. A., et al, Methods Enzymol., 2004, 388, 3-11; Nannemann, D. P, et al., Future Med Chem., 2011, 3, 809-819). In some embodiments, cell-free biosynthesis systems are used to facilitate the creation of mutational variants of lasso peptides by introducing non-natural amino acids into the core peptide sequence, followed by formation of the lasso structure using the cell-free biosynthesis methods for lasso peptide production as described above.

In various embodiments of the method for generating the library, the one or more components function to provide the lasso precursor peptide comprises the lasso precursor peptide. In some embodiments, the lasso precursor peptide comprises a sequence selected from the even number of SEQ ID Nos: 1-2630. In some embodiments, the one or more components function to provide the lasso precursor peptide comprises a polynucleotide encoding the lasso precursor peptide. In some embodiments, the polynucleotide encoding the lasso precursor peptide comprises a sequence selected from the odd number of SEQ ID Nos: 1-2630. In some embodiments, the polynucleotide comprises an open reading frame encoding the lasso peptide operably linked to at least one TX-TL regulatory element. In some embodiments, the at least one TX-TL regulatory element is known in the art.

In various embodiments of the method for generating the library, the one or more components function to process the lasso precursor peptide into the lasso peptide comprises one or more components function to provide a lasso peptidase activity in the CFB system. In some embodiments, the one or more components function to process the lasso precursor peptide into the lasso peptide comprises one or more components function to provide a lasso cyclase activity in the CFB system. In some embodiments, the one or more components function to process the lasso precursor peptide into the lasso peptide comprises one or more components function to provide a lasso peptidase activity and a lasso cyclase activity in the CFB system.

In various embodiments of the method for generating the library, the components function to provide the lasso peptidase activity in the CFB system comprise a lasso peptidase. In some embodiments, the components function to provide the lasso peptidase activity in the CFB system comprise a peptide or polypeptide having a sequence selected from peptide Nos: 1316-2336. In some embodiments, the components function to provide the lasso cyclase activity in the CFB system comprise a lasso cyclase. In some embodiments, the components function to provide the lasso cyclase activity in the CFB system comprise a peptide or polypeptide having a sequence selected from peptide Nos: 2337-3761. In some embodiments, the components function to provide the lasso peptidase activity in the CFB system comprise a polynucleotide encoding the lasso peptidase. In some embodiments, the components function to provide the lasso cyclase activity in the CFB system comprise a polynucleotide encoding the lasso cyclase.

In various embodiments of the method for generating the library, the one or more components function to process the lasso precursor peptide into the lasso peptide comprises one or more components function to provide a RRE. In some embodiments, the components function to provide the RRE in the CFB system comprise a peptide or polypeptide having a sequence selected from peptide Nos: 37624593. In some embodiments, the components function to provide the RRE in the CFB system comprise a polynucleotide encoding the RRE.

In alternative embodiments, CFB methods and systems enable in vitro cell-free transcription/translation systems (TX-TL) and function as rapid prototyping platforms for the synthesis, modification and identification of products, e.g., lasso peptides or lasso peptide analogs, from a minimal set of lasso peptide biosynthetic pathway components. In alternative embodiments, CFB systems are used for the combinatorial biosynthesis of lasso peptides or lasso peptide analogs, from a minimal set of lasso peptide biosynthetic pathway components, such as those provided in the present invention. In alternative embodiments, CFB systems are used for the rapid prototyping of complex biosynthetic pathways as a way to rapidly assess combinatorial designs for the synthesis of lasso peptides that bind to a specific biological target. In alternative embodiments, these CFB systems are multiplexed for high-throughput automation to rapidly prototype lasso peptide biosynthetic pathway genes and proteins, the lasso peptides they encode and synthesize, and lasso peptide analogs, such as the lasso peptides cited in the present invention. CFB methods and systems, including those involving the use of in vitro TX-TL, are described in Culler, S. et al., PCT Application WO2017/031399 A1, and is incorporated herein by reference.

In alternative embodiments, CFB methods and systems provided herein to produce lasso peptides and lasso peptide analogs from a minimal set of lasso peptide biosynthetic pathway components are used for the rapid identification and combinatorial biosynthesis of lasso peptide or lasso peptide analogs. An exemplary feature of this platform is that an unprecedented level of chemical diversity of lasso peptides and lasso peptide analogs can be created and explored. In alternative embodiments, combinatorial biosynthesis approaches are executed through the variation and modification of lasso peptide pathway genes, using different refactored lasso peptide gene cluster combinations, using combinations of genes from different lasso peptide gene clusters, using genes that encode enzymes that introduce chemical modifications before or after formation of the lasso peptide, using alternative lasso peptide precursor combinations (e.g., varied amino acids), using different CFB reaction mixtures, supplements or conditions, or by a combination of these alternatives.

Combinatorial CFB methods as provided herein can be used to produce libraries of new compounds, including lasso peptide libraries. For example, an exemplary refactored lasso peptide pathway can vary enzyme specificity at any step or add enzymes to introduce new functional groups and analogs at any one or more sites in a lasso peptide. Exemplary processes can vary enzyme specificity to allow only one functional group in a mixture to pass to the next step, thus allowing each reaction mixture to generate a specific lasso peptide analog. Exemplary processes can vary the availability of functional groups at any step to control which group or groups are added at that step. Exemplary processes can vary a domain of an enzyme to modify its specificity and lasso peptide analog created. Exemplary processes can add a domain of an enzyme or an entire enzyme module to add novel chemical reaction steps to the lasso peptide pathway.

In alternative embodiments, CFB methods and systems provided herein to produce lasso peptides and lasso peptide analogs from a minimal set of lasso peptide biosynthetic pathway components overcome a primary challenge in lasso peptide discovery—that many predicted lasso peptide gene clusters cannot be expressed under laboratory conditions in the native host, or when cloned into a heterologous host. In alternative embodiments, CFB methods and systems provided herein to produce lasso peptides and lasso peptide analogs from a minimal set of lasso peptide biosynthetic pathway components, including the use of cell extracts for in vitro transcription/translation (TX-TL) systems express novel lasso peptide biosynthetic gene clusters without the regulatory constraints of the cell. In alternative embodiments, some or all of the lasso peptide pathway biosynthetic genes are refactored to remove native transcriptional and translational regulation. In alternative embodiments, some or all of the lasso peptide pathway biosynthetic genes are refactored and constructed into operons on plasmids.

Metabolic modeling and simulation algorithms can be utilized to optimize conditions for the CFB process and to optimize lasso peptide production rates and yields in the CFB system. Modeling can also be used to design gene knockouts that additionally optimize utilization of the lasso peptide pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on shifting the primary metabolism towards more efficient production of lasso peptides and lasso peptide analogs.

One computational method for identifying and designing metabolic alterations favoring biosynthesis of a desired product is the OptKnock computational framework (Burgard et al., Biotechnol. Bioeng., 2003, 84, 647-657). OptKnock is a metabolic modeling and simulation program that suggests gene deletion or disruption strategies that result in genetically stable metabolic network which overproduces the target product. Specifically, the framework examines the complete metabolic and/or biochemical network in order to suggest genetic manipulations that lead to maximum production of a lasso peptide or lasso peptide analog. Such genetic manipulations can be performed on strains used to produce cell extracts for the CFB methods and processes provided herein. Also, this computational methodology can be used to either identify alternative pathways that lead to biosynthesis of a desired lasso peptide or used in connection with non-naturally occurring systems for further optimization of biosynthesis of a desired lasso peptide.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in different ways. Biological systems are designed through evolutionary mechanisms that have been restricted by fundamental constraints that all living systems face. Therefore, constraints-based modeling strategy embraces these general realities. Further, the ability to continuously impose further restrictions on a network model via the tightening of constraints results in a reduction in the size of the solution space, thereby enhancing the precision with which biosynthetic performance can be predicted.

Given the teachings and guidance provided herein, those skilled in the art will be able to apply various computational frameworks for metabolic modeling and simulation to design and implement biosynthesis of lasso peptides or lasso peptide analogs using cell extracts and the CFB methods and processes provided herein for the synthesis of lasso peptides and lasso peptide analogs from a minimal set of lasso peptide biosynthetic pathway genes. Such metabolic modeling and simulation methods include, for example, the computational systems exemplified above as SimPheny® and OptKnock. Those skilled in the art will know how to apply the identification, design and implementation of the metabolic alterations using OptKnock to any of such other metabolic modeling and simulation computational frameworks and methods well known in the art.

5.7 Methods for Screening for CFB Products

In certain aspects, provided herein are also methods for screening products produced by the CFB system and related methods provided herein, including methods for screening lasso peptide and/or lasso peptide analogs for those with desirable properties, such as therapeutic properties.

In some embodiments, provided herein are methods for screening candidate lasso peptide or lasso peptide analogs for binding affinity to a predetermined target. In some embodiments, the target is a cell surface molecule. In some embodiments, binding of the lasso peptide or lasso peptide analog to the target activates a signaling pathway in a cell. In some embodiments, binding of the lasso peptide or lasso peptide analog to the target inhibits a cellular signaling pathway. In some embodiments, the cellular signaling pathway can be intracellular and/or intercellular. In some embodiments, the activation and/or inhibition of the cellular signaling pathway is useful for treating or preventing a diseased condition in the cell. Accordingly, lasso peptides and lasso peptide analogs screened and selected herein can be suitable for treating or preventing the diseased condition in a subject.

In some embodiments, the method for screening lasso peptides or lasso peptide analogs comprises contacting a candidate lasso peptide with a target; and measuring the binding affinity between the lasso peptide or lasso peptide analog and the target. In some embodiments, the target is in purified form. In other embodiments, the target is present in a sample.

In some embodiments, the method for screening lasso peptides or lasso peptide analogs comprises contacting a candidate lasso peptide with a cell expressing the target; and detecting a signal associated with a cellular signaling pathway of interest from the cell. In some embodiments, the signaling pathway is inhibited by a candidate lasso peptide or lasso peptide analog. In other embodiments, the signaling pathway is activated by a candidate lasso peptide or lasso peptide analog. In particular embodiments, the target is G protein-couple receptors (GPCRs).

In some embodiments, the method for screening lasso peptides or lasso peptide analogs comprises contacting a candidate lasso peptide with a subject expressing the target; and measuring a signal associated with a phenotype of interest from the subject. In some embodiments, the phenotype is a disease phenotype.

In some embodiments, binding of the lasso peptide or lasso peptide analog to the target facilitates delivery of the lasso peptide or lasso peptide analog to the target. Accordingly, in some embodiments, the method for screening lasso peptides or lasso peptide analogs comprises contacting a candidate lasso peptide or lasso peptide analog with a target; and detecting localization of the lasso peptide or lasso peptide analog near the target. In some embodiments, the lasso peptide or lasso peptide analog is comprised within a larger molecule, and detecting localization of the lasso peptide or lasso peptide analog is performed by detecting the localization of such larger molecule or a portion thereof. In various embodiments, the larger molecule is a conjugate, a complex or a fusion molecule comprising the lasso peptide or lasso peptide analog. In some embodiments, detecting localization of the larger molecule comprising the lasso peptide or lasso peptide analog is performed by detecting a signal produced by such larger molecule. In some embodiments, detecting localization of the larger molecule comprising the lasso peptide or lasso peptide analog is performed by detecting an effect produced by such larger molecule. In some embodiments, the larger molecule comprises the lasso peptide and a therapeutic agent, and detecting localization of the larger molecule is performed by detecting a therapeutic effect of the therapeutic agent. In some embodiments, the therapeutic effect is in vivo. In other embodiments, the therapeutic effect is in vitro. Accordingly, lasso peptides and lasso peptide analogs screened and selected herein can be suitable for targeted delivery of a therapeutic agent to a target location within a subject.

In some embodiments, binding of the lasso peptide or lasso peptide analog to the target facilitates purifying the target from the sample. In some embodiments, the target is comprised in a sample, and binding of the lasso peptide or lasso peptide analog to the target facilitates detecting the target from the sample. In some embodiments, detecting the target from the sample is indicative of the presence of a phenotype of interest in a subject providing the sample. In some embodiments, the phenotype is a diseased phenotype. Accordingly, lasso peptides and lasso peptide analogs screened and selected herein can be suitable for diagnosing the disease from a subject.

In various embodiments, any method for screening for a desired enzyme activity, e.g., production of a desired product, e.g., such as a lasso peptide or lasso peptide analog, can be used. Any method for isolating enzyme products or final products, e.g., lasso peptides or lasso peptide analogs, can be used. In alternative embodiments, methods and compositions of the invention comprise use of any method or apparatus to detect a purposefully biosynthesized organic product, e.g., lasso peptide or lasso peptide analog, or supplemented or microbially-produced organic products (e.g., amino acids, CoA, ATP, carbon dioxide), by e.g., employing invasive sampling of either cell extract or headspace followed by subjecting the sample to gas chromatography or liquid chromatography often coupled with mass spectrometry.

In some embodiments, the methods of screening lasso peptides and lasso peptide analogs comprises screening lasso peptides and lasso peptide analogs from a lasso peptide library as provided herein. In alternative embodiments, the apparatus and instruments are designed or configured for High Throughput Screening (HTS) and analysis of products, e.g., lasso peptides or lasso peptide analogs, produced by CFB methods and processes as provided herein, by detecting and/or measuring the products, e.g., lasso peptides, either directly or indirectly, in soluble form by sampling a CFB cell-free extract or medium. For example, either the FastQuan™ High-Throughput LCMS System from Thermo Fisher (Waltham, Mass., USA) or the StreamSelect™ LCMS System from Agilent Technologies (Santa Clara, Calif., USA) can be used to rapidly assay and identify production of lasso peptides or lasso peptide analogs in a CFB process implemented using 96-well, 384-well, or 1536-well plates.

In alternative embodiments, CFB methods and processes are automatable and suitable for use with laboratory robotic systems, eliminating or reducing operator involvement, while providing for high-throughput biosynthesis and screening.

Also provided are methods for screening a lasso peptide or lasso peptide analog or a library of lasso peptides or lasso peptide analogs, produced by a CFB method or process, including the use of a TX-TL system, for an activity of interest. For example, the activity can be for a pharmaceutical, agricultural, nutraceutical, nutritional or animal veterinary or health and wellness function.

Also provided are methods for screening the CFB reaction mixture for: (i) a modulator of protein activity or metabolic function; (ii) a toxic metabolite, peptide or protein; (iii) an inhibitor of transcription or translation, comprising: (a) providing a CFB reaction mixture as described or provided herein, wherein the CFB reaction mixture comprises at least one protein-encoding nucleic acid which leads to the formation of a lasso peptide or lasso peptide analog; (b) providing a test compound; (c) combining or mixing the test compound with the CFB reaction mixture under conditions wherein the CFB reaction mixture initiates or completes transcription and/or translation, or modifies a molecule, optionally a protein, a small molecule, a natural product, a lasso peptide, or a lasso peptide analog, and, (d) determining or measuring any change in the functioning of the CFB reaction mixture, or the transcription and/or translation machinery, or in the formation of lasso peptide products, wherein determining or measuring a change in the protein activity, transcription or translation or metabolic function identifies the test compound as a modulator of that protein activity, transcription or translation or metabolic function.

Also provided are methods screening for: a modulator of protein activity, transcription, or translation or cell function; a toxic metabolite or a protein; a cellular toxin; an inhibitor or of transcription or translation, comprising: (a) providing a CFB method and a cell extract or TX-TL composition described herein, wherein the composition comprises at least one protein-encoding nucleic acid; (b) providing a test compound; (c) combining or mixing the test compound with the cell extract under conditions wherein the TX-TL extract initiates or completes transcription and/or translation, or modifies a molecule (optionally a protein, a small molecule, a natural product, natural product analog, a lasso peptide, or a lasso peptide analog) and (d) determining or measuring any change in the functioning or products of the extract, or the transcription and/or translation, wherein determining or measuring a change in the protein activity, transcription or translation or cell function identifies the test compound as a modulator of that protein activity, transcription or translation or cell function.

Also provided are methods for screening of lasso peptides or lasso peptide analogs produced in a CFB system, whereby the CFP reaction mixture is directly assayed for biological activity, or optionally lasso peptides and analogs are substantially isolated and purified, comprising: (a) providing a CFB reaction mixture with a cell extract as described herein, wherein the composition comprises at least one protein-encoding nucleic acid; (b) providing a lasso precursor peptide, lasso precursor peptide gene, lasso core peptide, or lasso core peptide gene; (c) combining or mixing the lasso precursor peptide, lasso precursor gene, lasso core peptide, or lasso core peptide gene with the cell extract under conditions wherein the lasso precursor peptide, lasso peptide gene, lasso core peptide, or lasso core peptide gene is converted to form a lasso peptide or lasso peptide analog, and (d) directly contacting the CFB reaction mixture, containing the products of transcription and/or translation, including lasso peptides or lasso peptide analogs, with a protein, enzyme, receptor, or cell, wherein a change in protein activity, transcription or translation, or cell function is measured and detected and identifies the lasso peptide or lasso peptide analog as a modulator of biological activity, such as protein binding, enzyme activity, cell surface receptor activity, or cell growth; or (e) optionally substantially isolating and purifying the lasso peptides or lasso peptide analogs and contacting the lasso peptides or lasso peptide analogs, with a protein, enzyme, receptor, or cell, wherein the biological activity or cell function is measured and detected and identifies the lasso peptide or lasso peptide analog as a modulator of biological activity, such as protein binding, enzyme activity, cell surface receptor activity, or cell growth.

5.8 Analysis and Isolation of Lasso Peptides and Lasso Peptide Analogs

Suitable purification and/or assays to test for the production of lasso peptides or lasso peptide analogs can be performed using well known methods. Suitable replicates such as triplicate CFB reactions, can be conducted and analyzed to verify lasso peptide production and concentrations. The final lasso peptide product and any intermediates, and other organic compounds, can be analyzed by methods such as HPLC (High Performance Liquid Chromatography), GC-MS (Gas Chromatography-Mass Spectrometry), LC-MS (Liquid Chromatography-Mass Spectrometry), MALDI or other suitable analytical methods using routine procedures well known in the art. Byproducts and residual amino acids or glucose can be quantified by HPLC using, for example, a refractive index detector for glucose and saturated fatty acids, and a UV detector for amino acids and other organic acids (Lin et al., Biotechnol. Bioeng, 2005, 90, 775-779), or other suitable assay and detection methods well known in the art. The individual enzyme or protein activities from the exogenous or endogenous DNA sequences can also be assayed using methods well known in the art. For example, the activity of phenylpyruvate decarboxylase can be measured using a coupled photometric assay with alcohol dehydrogenase as an auxiliary enzyme (See: Weiss et al., Biochem, 1988, 27, 2197-2205). NADH- and NADPH-dependent enzymes such as acetophenone reductase can be followed spectrophotometrically at 340 nm (See: Schlieben et al, J. Mol. Biol., 2005, 349, 801-813). For typical hydrocarbon assay methods, see Manual on Hydrocarbon Analysis (ASTM Manula Series, A. W. Drews, ed., 6th edition, 1998, American Society for Testing and Materials, Baltimore, Md.

Lasso peptides and lasso peptide analogs can be isolated, separated purified from other components in the CFB reaction mixtures using a variety of methods well known in the art. Such separation methods include, for example, extraction procedures, including extraction of CFB reaction mixtures using organic solvents such as methanol, butanol, ethyl acetate, and the like, as well as methods that include continuous liquid-liquid extraction, solid-liquid extraction, solid phase extraction, pervaporation, membrane filtration, membrane separation, reverse osmosis, electrodialysis, dialysis, distillation, crystallization, centrifugation, extractive filtration, ion exchange chromatography, size exclusion chromatography, adsorption chromatography, ultrafiltration, medium pressure liquid chromatography (MPLC), and high pressure liquid chromatography (HPLC). All of the above methods are well known in the art and can be implemented in either analytical or preparative modes.

5.9 Identifying and Modifying Lasso Peptide Biosynthetic Genes, Gene Clusters, Enzymes, and Pathways

Provided herein are methods of identifying and/or modifying an enzyme-encoding lasso peptide synthesizing operon; a lasso peptide biosynthetic gene cluster; a plurality of enzyme-encoding nucleic acids for lasso precursor peptides or lasso core peptides and at least one, several or all of the steps in the synthesis of a lasso peptide or lasso peptide analog upon transforming a lasso precursor peptide or lasso core peptide. In alternative embodiments, provided are engineered or modified enzyme-encoding lasso peptide synthesizing operons; lasso peptide biosynthetic gene clusters; and/or enzyme-encoding nucleic acids for lasso precursor peptides or lasso core peptides and at least one, several or all of the steps in the synthesis of a lasso peptide or lasso peptide analog upon transforming a lasso precursor peptide or lasso core peptide, or libraries thereof, made by these methods. In alternative embodiments, provided are libraries of lasso peptides or lasso peptide analogs made by these methods, and compositions as provided herein. In alternative embodiments, these modifications comprise one or more combinatorial modifications that result in generation of desired lasso peptides or lasso peptide analogs, or libraries of lasso peptides or lasso peptide analogs.

In alternative embodiments, the one or more combinatorial modifications comprise deletion or inactivation one or more individual genes, in a gene cluster for the biosynthesis, or altered biosynthesis, ultimately leading to a minimal optimum gene set for the biosynthesis of lasso peptides or lasso peptide analogs.

In alternative embodiments, the one or more combinatorial modifications comprise domain engineering to fuse protein (e.g., enzyme) domains, shuffled domains, adding an extra domain, exchange of one or more (multiple) domains, or other modifications to alter substrate activity or specificity of an enzyme involved in the biosynthesis or modification of the lasso peptides or lasso peptide analogs.

In alternative embodiments, the one or more combinatorial modifications comprise modifying, adding or deleting a “tailoring” enzyme that act after the biosynthesis of a core backbone of the lasso peptide or lasso peptide analog is completed, optionally comprising N-methyltransferases, O-methyltransferases, biotin ligases, glycosyltransferases, esterases, acylases, acyltransferases, aminotransferases, amidases, hydroxylases, dehydrogenases, halogenases, kinases, RiPP heterocyclases, RiPP cyclodehydratases, and prenyltransferases. In this embodiment, lasso peptides or lasso peptide analogs are generated by the action (e.g., modified action, additional action, or lack of action (as compared to wild type)) of the “tailoring” enzymes.

In alternative embodiments, the one or more combinatorial modifications comprise combining lasso peptide biosynthetic genes from various sources to construct artificial lasso peptide biosynthesis gene clusters, or modified lasso peptide biosynthesis gene clusters.

In alternative embodiments, functional or bioinformatic screening methods are used to discover and identify biocatalysts, genes and gene clusters, e.g., lasso peptide biosynthetic gene clusters, for use the CFB methods and processes as described herein. Environmental habitats of interest for the discovery of lasso peptides includes soil and marine environments, for example, through DNA sequence data generated through either genomic or metagenomic sequencing.

In alternative embodiments, enzyme-encoding lasso peptide synthesizing operons; lasso peptide biosynthetic gene clusters; and/or enzyme-encoding nucleic acids for lasso precursor peptides or lasso core peptides and at least one, several or all of the steps in the synthesis of a lasso peptide or lasso peptide analog upon transforming a lasso precursor peptide or lasso core peptide, or libraries thereof, made by the CFB methods and processes provided herein, are identified by methods comprising e.g., use of: a genomic or biosynthetic search engine, optionally WARP DRIVE BIO™ software, anti-SMASH (ANTI-SMASH™) software (See: Blin, K., et al., Nucleic Acids Res., 2017, 45, W36-W41), iSNAP™ algorithm (See: Ibrahim, A., et al., Proc. Nat. Acad Sci., USA., 2012, 109, 19196-19201), CLUSTSCAN™ (Starcevic, et al., Nucleic Acids Res., 2008, 36, 6882-6892), NP searcher (Li et al. (2009) Automated genome mining for natural products. BMC Bioinformatics, 10, 185), SBSPKS™ (Anand, et al. Nucleic Acids Res., 2010, 38, W487-W496), BAGEL3™ (Van Heel, et al., Nucleic Acids Res., 2013, 41, W448-W453), SMURF™ (Khaldi et al., Fungal Genet. Biol., 2010, 47, 736-741), ClusterFinder (CLUSTERFINDER™) or ClusterBlast (CLUSTERBLAST™) algorithms, the RODEO algorithm (See: Tietz, J. I., et al., Nature Chem Bio, 2017, 13, 470-478), or a combination there of, or, an Integrated Microbial Genomes (IMG)-ABC system (DOE Joint Genome Institute (JGI)).

In alternative embodiments, lasso peptide biosynthetic gene clusters for use in CFB methods and processes as provided herein are identified by mining genome sequences of known bacterial natural product producers using established genome mining tools, such as anti-SMASH, BAGEL3, and RODEO. These genome mining tools can also be used to identify novel biosynthetic genes (for use in CFB systems and processes as provided herein) within metagenomic based DNA sequences.

In alternative embodiments, CFB reaction mixtures and cell extracts as provided herein use (incorporate, or comprise) protein machinery that is responsible for the biosynthesis of secondary metabolites inside prokaryotic and eukaryotic cells; this “machinery” can comprise enzymes encoded by gene clusters or operons. In alternative embodiments, so-called “secondary metabolite biosynthetic gene clusters (SMBGCs) are used; they contain all the genes for the biosynthesis, regulation and/or export of a product, e.g., a lasso peptide. In vivo genes are encoded (physically located) side-by-side, and they can be used in this “side-by-side” orientation in (e.g., linear or circular) nucleic acids used in the CFB method and processes using cell extracts as provided herein, or they can be rearranged, or segmented into one or more linear or circular nucleic acids.

In alternative embodiments, the identified lasso peptide biosynthetic gene clusters and/or biosynthetic genes are ‘refactored’, e.g., where the native regulatory parts (e.g. promoter, RBS, terminator, codon usage etc.) are replaced e.g., by synthetic, orthogonal regulation with the goal of optimization of enzyme expression in a cell extract as provided herein and/or in a heterologous host (See: Tan, G.-Y., et al., Metabolic Engineering, 2017, 39, 228-236). In alternative embodiments, refactored lasso peptide biosynthetic gene clusters and/or genes are modified and combined for the biosynthesis of other lasso peptide analogs (combinatorial biosynthesis). In alternative embodiments, refactored gene clusters are added to a CFB reaction mixture with a cell extract as provided herein, and they can be added in the form of linear or circular DNA, e.g., plasmid or linear DNA.

In alternative embodiments, refactoring strategies comprise changes in a start codon, for example, for Streptomyces it might be beneficial to change the start codon, e.g., to TTG. For Streptomyces it has been shown that genes starting with TTG are better transcribed than genes starting with ATG or GTG (See: Myronovskyi et al., Applied and Environmental Microbiology, 2011; 77, 5370-5383).

In alternative embodiments, refactoring strategies comprise changes in ribosome binding sites (RBSs), and RBSs and their relationship to a promoter, e.g., promoter and RBS activity can be context dependent. For example, the rate of transcription can be decoupled from the contextual effect by using ribozyme-based insulators between the promoter and the RBS to create uniform 5′-UTR ends of mRNA, (See: Lou, et al., Nat. Biotechnol., 2012, 30, 1137-42.

In alternative embodiment, exemplary processes and protocols for the functional optimization of biosynthetic gene clusters by combinatorial design and assembly comprise methods described herein including next generation sequencing and identification of genes, genes clusters and networks, and gene recombineering or recombination-mediated genetic engineering (See: Smanski et al., Nat. Biotechnol., 2014, 32, 1241-1249).

In parallel, refactored linear DNA fragments can also be cloned into a suitable expression vector for transformation into a heterologous expression host or for use in CFB methods and processes, as provided herein. In alternative embodiments, provided are CFB methods and reactions comprising refactored gene clusters with single organism or mixed cell extracts.

In alternative embodiments, products of the CFB methods and processes, including CFB reaction mixtures, are subjected to a suite of “-omics” based approaches including: metabolomics, transcriptomics and proteomics, towards understanding the resulting proteome and metabolome, as well as the expression of lasso peptide biosynthetic genes and gene clusters. In alternative embodiments, lasso peptides produced within CFB reaction mixtures as provided herein are identified and characterized using a combination of high-throughput mass spectrometry (MS) detection tools as well as chemical and biological based assays. Following the characterization of the CFB produced lasso peptides, the corresponding biosynthetic genes and gene clusters may be cloned into a suitable vector for expression and scale up in a heterologous or native expression host. Production of lasso peptides can be scaled up in an in vitro bioreactor or using a fermentor involving a heterologous or native expression host.

In alternative embodiments, metagenomics, the analysis of DNA from a mixed population of organisms, is used to discover and identify biocatalysts, genes, and biosynthetic gene clusters, e.g., lasso peptide biosynthetic gene clusters. In alternative embodiments, metagenomics is used initially to involve the cloning of either total or enriched DNA directly from the environment (eDNA) into a host that can be easily cultivated (See: Handelsman, J., Microbiol. Mol. Biol. Rev., 2004, 68, 669-685). Next generation sequencing (NGS) technologies also can be used e.g., to allow isolated eDNA to be sequenced and analyzed directly from environmental samples (See: Shokralla, et al., Mol. Ecol. 2012, 21, 1794-1805).

As described herein the CFB methods and reaction mixtures can produce analogs of known compounds, for example lasso peptide analogs. Accordingly, CFB reaction mixture compositions can be used in the processes described herein that generate lasso peptide diversity. Methods provided herein include a cell free (in vitro)method for making, synthesizing or altering the structure of a lasso peptide or lasso peptide analog, or a library thereof, comprising using the CFB reaction mixture compositions and CFB methods described herein. The CFB methods can produce in the CFB reaction mixture at least two or more of the altered lasso peptides to create a library of altered lasso peptides; preferably the library is a lasso peptide analog library, prepared, synthesized or modified by a CFB method comprising use of the cell extracts or extract mixtures described herein or by using the processor method described herein. Also provided is a library of lasso peptides or lasso peptide analogs, or a combination thereof, prepared, synthesized or modified by a CFB method comprising a CFB reaction mixture that produces lasso peptides or lasso peptide analogs from a minimal set of lasso peptide biosynthesis components, as described herein or by using the process or method described herein.

In alternative embodiments, practicing the invention comprises use of any conventional technique commonly used in molecular biology, microbiology, and recombinant DNA, which are within the skill of the art. Such techniques are known to those of skill in the art and are described in numerous texts and reference works (See e.g., Sambrook et al., “Molecular Cloning: A Laboratory Manual,” Second Edition, Cold Spring Harbor, 1989; and Ausubel et al., “Current Protocols in Molecular Biology,” 1987). Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, N Y (1994); and Hale and Marham, The Harper Collins Dictionary of Biology, Harper Perennial, NY (1991) provides those of skill in the art with general dictionaries of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole.

5.10 Conjugation

In alternative embodiments, CFB methods and systems, including those involving in vitro, or cell-free, transcription/translation (TX-TL), are used to produce a lasso peptide or lasso peptide analog that is fused or conjugated to a second molecule or molecules, optionally a pharmaceutically acceptable carrier molecule, optionally a polymer, a protein or peptide, an antibody or fragment thereof, an affibody, a nanobody, a PEG or a PEG derivative, a lipophilic carrier including a fatty acid, optionally palmitoyl, myristoyl, stearic acid, 3-pentadecylglutaric acid, that associates with a serum protein such as albumin, LDL or HDL, and wherein optionally the carrier increases blood circulation time or cell-targeting or both for the lasso peptide or lasso peptide analog; and optionally the lasso peptide or lasso peptide analog is fused or conjugated to a second molecule or molecules in the cell extract, and optionally is enriched before being fused or conjugated to the second molecule or molecules, or is isolated before being fused or conjugated to the second molecule or molecules, and optionally the lasso peptide or lasso peptide analog is site-specifically fused or conjugated to the second molecule or molecules, optionally wherein the lasso peptide or lasso peptide analog is modified to comprise a group capable of the site-specific fusion or conjugation to the second molecule or molecules, optionally where the lasso peptide or lasso peptide analog is synthesized in the CFB reaction mixture to comprise the site-specific reactive group, and, optionally wherein the library contains a plurality of lasso peptides or lasso peptide analogs, each having a site-specific reactive group at a different location on the lasso peptide or lasso peptide analogs, and optionally the site-specific reactive group can react with a cysteine or lysine or serine or tyrosine or glutamic acid or aspartic acid or azide or alkyne or alkene on the second molecule or molecules.

In alternative embodiments, provided are methods and compositions comprising: a lasso peptide or lasso peptide analog, obtained from a library as provided herein, wherein optionally the composition further comprises, is formulated with, or is contained in: a liquid, a solvent, a solid, a powder, a bulking agent, a filler, a polymeric carrier or stabilizing agent, a liposome, a particle or a nanoparticle, a buffer, a carrier, a delivery vehicle, or an excipient, optionally a pharmaceutically acceptable excipient.

In alternative embodiments, a lasso peptide or lasso peptide analog is fused or conjugated to a second molecule, optionally a pharmaceutically acceptable carrier molecule, optionally a polymer, a protein or peptide, an antibody or fragment thereof, an affibody, a nanobody, a PEG or a PEG derivative, biotin, a lipophilic carrier including a fatty acid, optionally palmitoyl, myristoyl, steric acid, 3-pentadecylglutaric acid, that associates with a serum protein such as albumin, LDL or HDL, and wherein optionally the carrier increases blood circulation time or cell-targeting or both for the lasso peptide or lasso peptide analog. In alternative embodiments, the lasso peptide or lasso peptide analog is fused or conjugated to the second molecule or molecules in the cell extract, and optionally is enriched before being fused or conjugated to the second molecule or molecules, or is isolated before being fused or conjugated to the second molecule or molecules.

In alternative embodiments, a lasso peptide or lasso peptide analog is site-specifically fused or conjugated to the second molecule, optionally wherein the lasso peptide or lasso peptide analog is modified to comprise a group capable of the site-specific fusion or conjugation to the second molecule or molecules, optionally where the lasso peptide or lasso peptide analog is synthesized in the cell extract to comprise the site-specific reactive group, and optionally wherein the library contains a plurality of lasso peptides or lasso peptide analogs each having a site-specific reactive group at a different location on the lasso peptide or lasso peptide analog, and optionally the site-specific reactive group can react with a cysteine or lysine or seine or tyrosine or glutamic acid or aspartic acid or azide or alkyne or alkene on the second molecule or molecules.

In alternative embodiments, provided are in vitro methods for making, synthesizing or altering the structure of a lasso peptide or lasso peptide analog, or library thereof, comprising use of a CFB reaction mixture with a cell extract as provided herein, or by using a CFB method or system as provided herein. In alternative embodiments, at least two or more of the altered lasso peptides are synthesized to create a library of altered lasso peptide variants, and optionally the library is a lasso peptide analog library.

In alternative embodiments, provided are libraries of lasso peptide or lasso peptide analogs, or a combination thereof, prepared, synthesized or modified by a CFB method or system comprising use of a CFB reaction mixture with a cell extract as provided herein, or by using a CFB method or system as provided herein. In alternative embodiments, the method for preparing, synthesizing or modifying the lasso peptide or lasso peptide analogs, or the combination thereof, comprises using a CFB reaction mixture with a cell extract from an Escherichia or from an Actinomyces, optionally a Streptomyces.

In alternative embodiments of the libraries: the lasso peptides or lasso peptide analogs, are site-specifically fused or conjugated to a second molecule or molecules; optionally wherein the lasso peptides or lasso peptide analogs are modified to comprise a group capable of the site-specific fusion or conjugation to the second molecule or molecules, optionally where the lasso peptides or lasso peptide analogs are synthesized in the CFB reaction mixture containing a cell extract to comprise the site-specific reactive group, and optionally wherein the library contains a plurality of lasso peptides or lasso peptide analogs, each having a site-specific reactive group at a different location on the lasso peptides or lasso peptide analogs, and optionally the site-specific reactive group can react with a cysteine or lysine or serine or tyrosine or glutamic acid or aspartic acid or azide or alkyne or alkene on the second molecule or molecules.

In alternative embodiments, the invention provides a method or composition according to any embodiment of the invention, substantially as herein before described, or described herein, with reference to any one of the examples. In alternative embodiments, practicing the invention comprises use of any conventional technique commonly used in molecular biology, microbiology, and recombinant DNA, which are within the skill of the art. Such techniques are known to those of skill in the art and are described in numerous texts and reference works (See e.g., Green and Sambrook, “Molecular Cloning: A Laboratory Manual,” 4th Edition, Cold Spring Harbor, 2012; and Ausubel et al., “Current Protocols in Molecular Biology,” 1987). Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, N Y (1994); and Hale and Marham, The Harper Collins Dictionary of Biology, Harper Perennial, NY (1991) provides those of skill in the art with general dictionaries of many of the terms used in the invention. Although any methods and materials similar or equivalent to those described herein find use in the practice of the present invention, the preferred methods and materials are described herein. Accordingly, the terms defined below are more fully described by reference to the Specification as a whole.

### 6. EXAMPLES

Examples related to the present invention are described below. In most cases, alternative techniques can be used. The examples are intended to be illustrative and are not limiting or restrictive to the scope of the invention. For example, where lasso peptides or lasso peptide analogs are prepared following a protocol of a Scheme, it is understood that conditions may vary, for example, any of the solvents, reaction times, reagents, temperatures, supplements, work up conditions, or other reaction parameters may be varied.

### General Methods

All molecular biology and cell-free biosynthesis reactions were conducted using standard plates, vial, and flasks typically employed when working with biological molecules such as DNA, RNA and proteins. LC-MS/MS analyses (including Hi-Res analysis) were performed on an Agilent 6530 Accurate-Mass Q-TOF MS equipped with a dual electrospray ionization source and an Agilent 1260 LC system with diode array detector. MS and UV data were analyzed with Agilent MassHunter Qualitative Analysis version B.05.00. All MALDI-TOF analyses were performed using a Bruker UltrafleXtreme MALDI TOF/TOF mass spectrometer. Preparative HPLC was carried out using an Agilent 218 purification system (ChemStation software, Agilent) equipped with a ProStar 410 automatic injector, Agilent ProStar UV-Vis Dual Wavelength Detector, a 440-LC fraction collector and preparative HPLC column indicated below. Semi-preparative HPLC purifications were performed on an Agilent 1260 Series Instrument with a multiple wavelength detector and Phenomenex Luna 5 μm C8(2) 250×100 mm semi preparative column. Unless otherwise specified, all HPLC purifications utilized 10 mM aq. NH4HCO3/MeCN and all analytical LCMS methods included a 0.1% formic acid buffer. NMR data are acquired using a 600 MHz Bruker Avance III spectrometer with a 1.7 mm cryoprobe. All signals are reported in ppm with the internal DMSO-d6 signal at 2.50 ppm (¹H-NMR) or 39.52 ppm (¹³C-NMR). 1D data is reported as s=singlet, d=doublet, t=triplet, q=quadruplet, m=multiplet or unresolved, br=broad signal, coupling constant(s) in Hz.

To prepare cell extracts, E. coli BL21 Star(DE3) cells were grown in the minimum medium containing MM9 salts (13 g/L), calcium chloride (0.1 mM), magnesium sulfate (2 mM), trace elements (2 mM) and glucose (10 g/L), in a 10 L bioreactor (Satorius) to the mid-log growth phase. The grown cells were then harvested and pelleted. The crude cell extracts were prepared as described in Kay, J., et al., Met. Eng., 2015, 32, 133-142 and Sun, Z. Z., J. Vis. Exp. 2013, 79, e50762, doi:10.3791/50762. For calibration of additional magnesium, potassium and DTT levels, a green fluorescence protein (GFP) reporter was used to determine the additional amount of Mg-glutamate, K-glutamate, and DTT that were subsequently added to each batch of the crude cell extracts to prepare the optimized cell extracts for optimal transcription-translation activities. Prior to cell-free biosynthesis of lasso peptide, the optimized cell extracts were pre-mixed with buffer that contains ATP, GTP, TTP, CTP, amino acids, t-RNA, magnesium glutamate, potassium glutamate, potassium phosphate, and other salts, NAD+, NADPH, glucose, 500 uM IPTG and 3 mM DTT to achieve a desirable reaction volume. An exemplary cell extract comprises the ingredients, and optionally with the amounts, as set forth in the following Table X1.

TABLE Xl

| Ingredients | Concentration | |
|---|---|---|
| E. coli BL21 | 33% v/v | |
| Star(DE3) extracts | (10 mg/ml of protein or higher) | |
| Amino Acids | 1.5 mM each | |
| | (Leucine, 1.25 mM) | |
| HEPES | 50 | mM |
| ATP | 1.5 | mM |
| GTP | 1.5 | mM |
| CTP & UTP | 0.9 | mM |
| tRNA | 0.2 | mg/mL |
| CoA | 0.26 | mM |
| NAD+ | 0.33 | mM |
| cAMP | 0.75 | mM |
| Folinic acid | 0.068 | mM |
| spermidine | 1 | mM |
| pEG-8000 | 2% | |
| magnesium glutamate | 4-12 | mM |
| potassium glutamate | 8-160 | mM |
| potassium phosphate | 1-10 | mM |
| DTT | 0-5 | mM |
| NADPH | 1 | mM |
| maltodexttin | 35 | mM |
| IPTG (optional) | 0.5 | mM |
| pyruvate | 30 | mM |
| NADH | 1 | mM |

Affinity chromatography procedures are carried out according to the manufacturers' recommendations to isolate lasso peptides fused to an affinity tag; for examples, Strep-tag® II based affinity purification (Strep-Tactin® resin, IBA Lifesciences), His-tag-based affinity purification (Ni-NTA resin, ThermoFisher), maltose-binding protein based affinity purification (amylose resin, New England BioLabs). The sample of lasso peptides fused to an affinity tag is lyophilized and resuspended in a binding buffer with respect to its affinity tag according to the manufacturer's recommendation. The resuspended lasso peptide sample is directly applied to an immobilized matrix corresponding to its fused affinity tag (Tactin for Strep-tag® II, Ni-NTA for His-tag, or amylose resin for maltose binding protein) and incubated at 4° C. for an hour. The matrix is then washed with at least 40× volume of washing buffer and eluted with three successive 1× volume of elution buffer containing 2.5 mM desthiobiotin for Strep-Tactin® resin, 250 mM imidizole for Ni-NTA resin or 10 mM maltose for amylose resin. The eluted fractions are analyzed on a gradient (10-20%) Tris-Tricine SDS-PAGE gel (Mini-PROTEAN, BioRad) and then stained with Coomassie brilliant blue.

The purity of eluted lasso peptide was examined by LC-MSMS on an Agilent 6530 Accurate-Mass Q-TOF mass spectrometer. Where possible, MSMS fragmentation is used to further characterize lasso peptides based on the rule described in Fouque, K. J. D, et al., Analyst, 2018, 143, 1157-1170. If impurities are observed in chromatographic spectra, preparative chromatography is performed to further enrich the purity of lasso peptides.

### Analytical LCMS Analytical Method:

Column: Phenomenex Kinetex 2.6 XB-C18 100 A, 150×4.6 mm column.
Flow rate: 0.7 mL/min
Temperature: RTMobile Phase A: 0.1% formic acid in water
Mobile Phase B: 0.1% formic acid in acetonitrile
Injection amount: 2 □L
HPLC Gradient: 10% B for 3.0 min, then 10 to 100% B over 20 minutes follow by 100% B for 3 min. 4 minute post run equilibration time

Preparative HPLC was carried out using an Agilent 218 purification system (ChemStation software, Agilent) equipped with a ProStar 410 automatic injector, Agilent ProStar UV-Vis Dual Wavelength Detector, a 440-LC faction collector. Fractions containing lasso peptides were identified using the LCMS method described above, or by direct injection (bypassing the LC column in the above method) prior to combining and freeze-drying. Analytical LC/MS (see method above) was then performed on the combined and concentrated lasso peptides.

### Preparative HPLC Method:

Column: Phenomenex Luna® preparative column 5 μM, C8(2) 100 Å100×21.2 mm
Flow rate: 15 m/min

### Temperature: RT

Mobile Phase A: 10 mM aq. NH4HCO3
Mobile Phase B: acetonitrile
Injection amount: vanes
HPLC Gradient: 20-40% MeCN for 20 min, then 40-95% MeCN for 5 min

If necessary, semi-preparative HPLC purifications were performed on an Agilent 1260 Series Instrument with a multiple wavelength detector

Semipreparative HPLC Method:

Column: Phenomenex Luna®5 μm C18(2)250×100 mm

Flow rate: 4 m/min

Temperature: RT

Mobile Phase A: 10 mM aq. NH4HCO3

Mobile Phase B: acetonitrile

Injection amount: vanes

HPLC Gradient: 20-40% MeCN for 20 min, then 40-95% MeCN for 5 min

Monoisotopic masses were extrapolated from the lasso peptide charge envelop [(M+H)¹⁺, (M+2H)²⁺, (M+3H)³⁺] in the m/z 500-3,200 range using a Agilent 6530 Accurate-Mass Q-TOF MS equipped with a dual electrospray ionization source and an Agilent 1260 LC system using an internal reference (see analytical procedure described above). Both MS and MS/MS analyses were performed in positive-ion mode.

NMR samples are dissolved in DMSO-d6 (Cambridge Isotope Lab-oratories). All NMR experiments are run on a 600 MHz Bruker Avance III spectrometer with a 1.7 mm cryoprobe. All signals are reported in ppm with the internal DMSO-d6 signal at 2.50 ppm (H-NMR) or 39.52 ppm (¹³C-NMR). Where applicable, structural characterization of lasso peptide follow the methods described in the literatures listed below:
1. Knappe et al., J. Am. Chem. Soc., 2008, 130 (34), 11446-11454
2. Maksimov et al., PNAS, 2012, 109 (38), 15223-15228
3. Tietz et al., Nature Chem. Bio., 2017, 13, 470-478
4. Zheng and Price, Prog Nucl Magn Reson Spectrosc, 2010, 56 (3), 267-288
5. Marion et al., J Magn Reson, 1989, 85 (2), 393-399
6. Davis et al., J Magn Reson, 1991, 94 (3), 637-644
7. Rucker and Shaka, Mol Phys, 1989, 68 (2), 509-517
8. Hwang and Shaka, J Magn Reson A, 1995, 112 (2), 275-27

Table X2 below lists examples of lasso peptides produced with cell-free biosynthesis using a minimum set of genes.

TABLE X2
minimum set of genes required for cell-free biosynthesis of lasso peptides

| | | Precursor | Peptidase | Cyclase | Cyclase- | RRE | RRE- |
|---|---|---|---|---|---|---|---|
| Lasso | Molecular | peptide | peptide | peptide | RRE | peptide | peptidase |
| peptide | mass | No: | No: | No: | peptide No: | No: | peptide No: |
| microcin J25 | 2107 | 92 | 1492 | 2571 | — | — | — |
| ukn22 | 2269 | 525 | 1584 | 2676 | — | 3975 | — |
| capistruin | 2049 | 15 | 1566 | 3438 | — | — | — |
| lariatin | 2204 | 162 | 1368 | 2406 | — | 3803 | — |
| ukn16 | 2306 | 823 | 1442 | — | 2504 | — | — |
| adanomysin | 1676 | 839 | — | 3128 | — | — | 4150 |
| burhizin | 1848 | 111 | 2033 | 2722 | — | — | — |
| cellulonodin | 2277 | 2645 | 2647 | 2649 | — | 2651 | — |

Table X3 below lists the amino acid sequence of ukn22 lasso peptide and ukn22 lasso peptide variants produced with cell-free biosynthesis.

TABLE X3
amino acid sequence of ukn22
lasso peptide and ukn22 lasso
peptide variants

| | | Amino acid |
|---|---|---|
| | | sequence of |
| Lasso | Molecular | the core |
| peptide | mass | lasso peptide |
| ukn22 | 2269 | WYTAEWGLELIFVFPRFI |
| | | (SEQ ID NO: 2632) |
| ukn22 W1Y | 2246 | YYTAEWGLELIFVFPRFI |
| | | (SEQ ID NO: 2638) |
| ukn22 W1F | 2230 | FYTAEWGLELIFVFPRFI |
| | | (SEQ ID NO: 2639) |
| ukn22 W1H | 2220 | HYTAEWGLELIFVFPRFI |
| | | (SEQ ID NO: 2640) |
| ukn22 W1L | 2196 | LYTAEWGLELIFVFPRFI |
| | | (SEQ ID NO: 2641) |
| ukn22 W1A | 2154 | AYTAEWGLELIFVFPRFI |
| | | (SEQ ID NO: 2642) |

### Example 1

This study demonstrates synthesis of microcin J25 (MccJ25) lasso peptide GGAGHVPEYFVGIGTPISFYG (the lasso peptide of peptide No: 92) (SEQ ID NO: 2631) where the N-terminal amine group of a glycine (G) residue at the first position was cyclized with the side-chain carboxylic acid group of a glutamic acid (E) residue at the eighth position

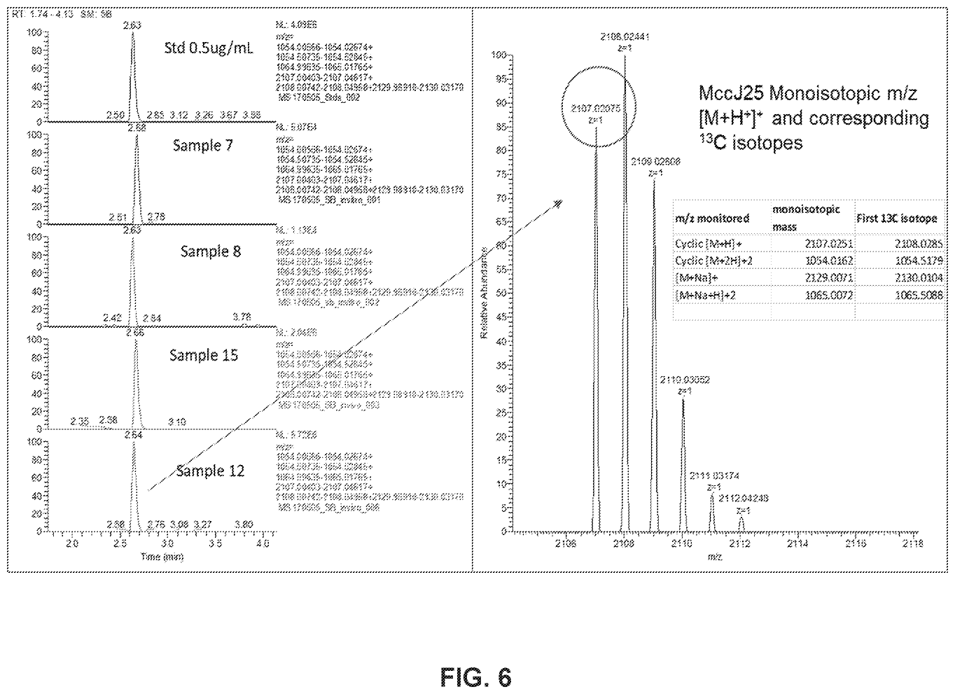

DNA encoding the sequences for the MccJ25 precursor peptide (peptide No: 92), peptidase (peptide No: 1492), and cyclase (peptide No: 2571) from Escherichia coli were synthesized (Thermo Fisher, Carlsbad, Calif.) and individually cloned into a pZE expression vector behind a T7 promoter (Expressys). The resulting plasmids encoding genes for the MccJ25 precursor peptide (peptide No: 92) without a C-terminal affinity tag, peptidase (peptide No: 1492) with a C-terminal Strep-tag®, and cyclase (peptide No: 2571) also with a C-terminal Strep-tag® were used for subsequent cell-free biosynthesis. The MccJ25 precursor peptide (peptide No: 92) was produced using the PURE system (New England BioLabs) according to the manufacturer's recommended protocol. The peptidase (peptide No: 1492) and cyclase (peptide No: 2571) were expressed in Escherichia coli as described by Yan et al., Chembiochem. 2012, 13(7):1046-52 (doi: 10.1002/cbic.201200016) and purified using Tactin resin (IBA Lifesciences) according to the manufacturer's recommendation. Production of MccJ25 lasso peptide was initiated by adding 5 μL of the PURE reaction containing the MccJ25 precursor peptide (peptide No: 92), and 10 μL of purified peptidase (peptide No: 1492), and 20 μL of purified cyclase (peptide No: 2571) in buffer that contains 50 mM Tris (pH8), 5 mM MgCl2, 2 mM DTT and 1 mM ATP to achieve a total volume of 50 μL. The cell-free biosynthesis of MccJ25 lasso peptide was accomplished by incubating the reaction for 3 hours at 30° C. The reaction sample was subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid fraction was subjected to LC/MS analysis on an Applied Biosystems 3200 APCI triple quadrupole mass spectrometer for lasso peptide detection. The molecular mass of 2107.02 m/z corresponding to MccJ25 lasso peptide (GGAGHVPEYFVGIGTPISFYG (SEQ ID NO: 2631) minus H₂O) was observed and compared to an authentic sample (Std) of MccJ25 (FIG. 6).

### Example 2

This study demonstrates synthesis of ukn22 lasso peptide WYTAEWGLELIFVFPRFI (the lasso peptide of peptide No: 525) (SEQ ID NO: 2632) where the N-terminal amine group of a tryptophan (W) residue at the first position was cyclized with the side-chain carboxylic acid group of a glutamic acid (E) residue at the ninth position.

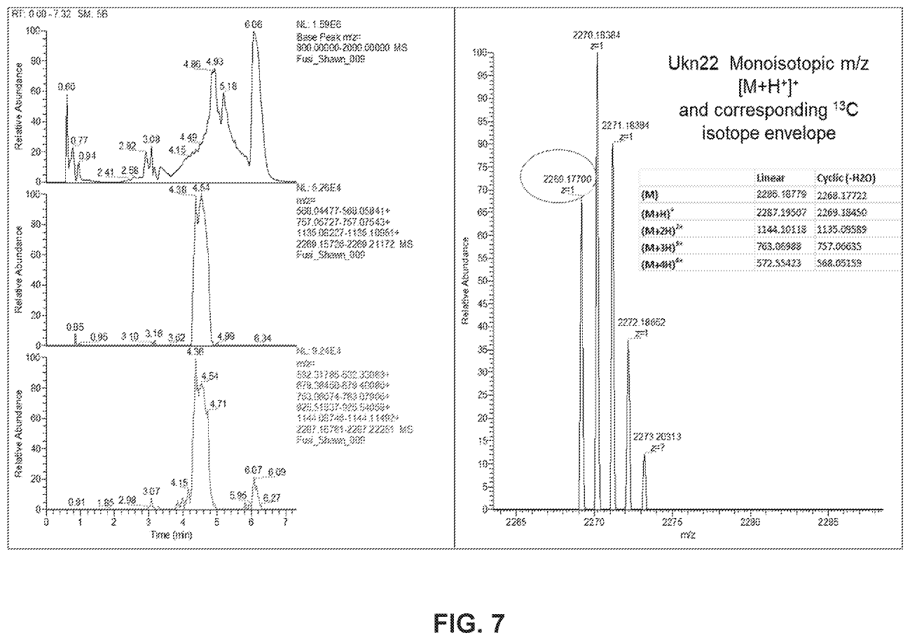

DNA encoding the sequences for the ukn22 precursor peptide (peptide No: 525), peptidase (peptide No: 1584), cyclase (peptide No: 2676) and RRE (peptide No: 3975) from Thermobifida fusca were used. Each of the DNA sequences was cloned into a pET28 plasmid vector behind a maltose binding protein (MBP) sequence to create an N-terminal MBP fusion protein. The resulting plasmids encoding fusion genes for the MBP-ukn22 precursor peptide (peptide No: 525), MBP-peptidase (peptide No: 1584), MBP-cyclase (peptide No: 2676) and MBP-RRE (peptide No: 3975) were driven by an IPTG-inducible T7 promoter. Production of ukn22 lasso peptide was initiated by adding the plasmid vectors encoding MBP-ukn22 precursor peptide (peptide No: 525), MBP-peptidase (peptide No: 1584), MBP-cyclase (peptide No: 2676) and MBP-RRE (peptide No: 3975) (20 nM each) to the optimized E. coli BL21 Star(DE3) cell extracts, which were pre-mixed with buffer as described earlier to achieve a total volume of 50 μL. The cell-free biosynthesis of ukn22 lasso peptide was accomplished by incubating the reaction for 16 hours at 22° C. The reaction sample was subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction was subjected to LC/MS analysis on an Applied Biosystems 3200 APCI triple quadrupole mass spectrometer for lasso peptide detection. The molecular mass of 2269.18 m/z corresponding to ukn22 lasso peptide (WYTAEWGLELIFVFPRFI (SEQ ID NO: 2632) minus H₂O) was observed (FIG. 7).

### Example 3

Synthesis of capistruin lasso peptide GTPGFQTPDARVISRFGFN (SEQ ID NO: 2633) (the lasso peptide of peptide No: 15) by adding the individually cloned genes for the capistruin precursor peptide (peptide No: 15), peptidase (peptide No: 1566) and cyclase (peptide No: 3438) where the N-terminal amine group of a glycine (G) residue at the first position is cyclized with the side-chain carboxylic acid group of an aspartic acid (D) residue at the ninth position.

Codon-optimized DNA encoding the sequences for the capistruin precursor peptide (peptide No: 15), peptidase (peptide No: 1566) and cyclase (peptide No: 3438) from Burkholderia thailandensis are synthesized (Thermo Fisher, Carlsbad, Calif.) and individually cloned into a pZE expression vector behind a T7 promoter (Expressys). The resulting plasmids encoding genes for the capistruin precursor peptide (peptide No: 15), peptidase (peptide No: 1566) and cyclase (peptide No: 3438) are used with or without a C-terminal affinity tag. Production of capistruin lasso peptide is initiated by adding the plasmid encoding the capistruin precursor peptide (peptide No: 15), peptidase (peptide No: 1566) and cyclase (peptide No: 3438) (15 nM each) to the optimized E. coli BL21 Star(DE3) cell extracts, which are pre-mixed with buffer that contains ATP, GTP, TTP, CTP, amino acids, t-RNA, magnesium glutamate, potassium glutamate, potassium phosphate, and other salts, NAD+, NADPH, and glucose to achieve a total volume of 400 μL. The cell-free biosynthesis of capistruin lasso peptide is accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid fraction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2049 m/z corresponding to capistruin lasso peptide (GTPGFQTPDARVISRFGFN (SEQ ID NO: 2633) minus H₂O) is observed. The collected lasso peptide sample is further purified by affinity chromatography and/or preparative HPLC, followed by high resolution mass spectrometry and NMR for structural characterization.

### Example 4

Synthesis of lariatin lasso peptide GSQLVYREWVGHSNVIKPGP (SEQ ID NO: 2634) (the lasso peptide of peptide No: 162) where the N-terminal amine group of a glycine (G) residue at the first position is cyclized with the side-chain carboxylic acid group of a glutamic acid (E) residue at the eighth position

Codon-optimized DNA encoding the sequences for the lariatin precursor peptide (peptide No: 162), peptidase (peptide No: 1368), cyclase (peptide No: 2406) and RRE (peptide No: 3803) from Rhodococcus jostii are synthesized (Thermo Fisher, Carlsbad, Calif.) and individually cloned into a pZE expression vector behind a T7 promoter (Expressys). The resulting plasmids encoding genes for the lariatin precursor peptide (peptide No: 162), peptidase (peptide No: 1368), cyclase (peptide No: 2406) and RRE (peptide No: 3803) are used with or without a C-terminal affinity tag. Production of lariatin lasso peptide is initiated by adding the plasmids encoding the lariatin precursor peptide (peptide No: 162), peptidase (peptide No: 1368), cyclase (peptide No: 2406) and RRE (peptide No: 3803) (15 nM each) to the optimized E. coli BL21 Star(DE3) cell extracts, which are pre-mixed with buffer that contains ATP, GTP, TTP, CTP, amino acids, t-RNA, magnesium glutamate, potassium glutamate, potassium phosphate, and other salts, NAD+, NADPH, and glucose to achieve a total volume of 400 μL. The cell-free biosynthesis of lariatin lasso peptide is accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2204 m/z corresponding to lariatin lasso peptide (GSQLVYREWVGHSNVIKPGP (SEQ ID NO: 2634) minus H₂O) is observed. The collected lasso peptide sample is further purified by affinity chromatography and/or preparative HPLC, followed by high resolution mass spectrometry and NMR for structural characterization.

### Example 5

Synthesis of ukn16 lasso peptide GVWFGNYVDVGGAKAPFPWGSN (SEQ ID NO: 2635)(the lasso peptide of peptide No: 823) where the N-terminal amine group of a glycine (G) residue at the first position is cyclized with the side-chain carboxylic acid group of an aspartic acid (D) residue at the ninth position

Codon-optimized DNA encoding the sequences for the ukn16 precursor peptide (peptide No: 823), peptidase (peptide No: 1442), and cyclase-RRE fusion protein (peptide No: 2504) from Bifidobacterium reuteri DSM 23975 are synthesized (Thermo Fisher, Carlsbad, Calif.) and individually cloned into a pZE expression vector behind a T7 promoter (Expressys). The resulting plasmids encoding genes for the ukn16 precursor peptide (peptide No: 823), peptidase (peptide No: 1442), and cyclase-RRE fusion protein (peptide No: 2504) are used with or without a C-terminal affinity tag. Production of ukn16 lasso peptide is initiated by adding the plasmids encoding the ukn16 precursor peptide (peptide No: 823), peptidase (peptide No: 1442), and cyclase-RRE fusion protein (peptide No: 2504) (15 nM each) to the optimized E. coli BL21 Star(DE3) cell extracts, which are pre-mixed with buffer that contains ATP, GTP, TTP, CTP, amino acids, t-RNA, magnesium glutamate, potassium glutamate, potassium phosphate, and other salts, NAD+, NADPH and glucose to achieve a total volume of 400 μL. The cell-free biosynthesis of ukn16 lasso peptide is accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2306 m/z corresponding to ukn16 lasso peptide (GVWFGNYVDVGGAKAPFPWGSN (SEQ ID NO: 2635) minus H₂O) is observed. The collected lasso peptide sample is further purified by affinity chromatography and/or preparative HPLC, followed by high resolution mass spectrometry and NMR for structural characterization.

### Example 6

Synthesis of adanomysin lasso peptide GSSTSGTADANSQYYW (the lasso peptide of peptide No: 839) (SEQ ID NO: 2636) where the N-terminal amine group of a glycine (G) residue at the first position is cyclized with the side-chain carboxylic acid group of an aspartic acid (D) residue at the ninth position

Codon-optimized DNA encoding the sequences for the adanomysin precursor peptide (peptide No: 839), cyclase (peptide No: 3128), and RRE-peptidase fusion protein (peptide No: 4150) from Streptomyces niveus are synthesized (Thermo Fisher, Carlsbad, Calif.) and individually cloned into a pZE expression vector behind a T7 promoter (Expressys). The resulting plasmids encoding genes for the adanomysin precursor peptide (peptide No: 839), cyclase (peptide No: 3128), and RRE-peptidase fusion protein (peptide No: 4150) are used with or without a C-terminal affinity tag. Production of adanomysin lasso peptide is initiated by adding the plasmids encoding the adanomysin precursor peptide (peptide No: 839), cyclase (peptide No: 3128), and RRE-peptidase fusion protein (peptide No: 4150) (15 nM each) to the optimized E. coli BL21 Star(DE3) cell extracts, which are pre-mixed with buffer that contains ATP, GTP, TTP, CTP, amino acids, t-RNA, magnesium glutamate, potassium glutamate, potassium phosphate, and other salts, NAD+, NADPH, and glucose to achieve a total volume of 400 μL. The cell-free biosynthesis of adanomysin lasso peptide is accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 1676 m/z corresponding to adanomysin lasso peptide (GSSTSGTADANSQYYW (SEQ ID NO: 2636) minus H₂O) is observed. The collected lasso peptide sample is further purified by affinity chromatography and/or preparative HPLC, followed by high resolution mass spectrometry and NMR for structural characterization.

### Example 7

Synthesis of ukn22 lasso peptide WYTAEWGLELIFVFPRFI (SEQ ID NO: 2632)(the lasso peptide of peptide No: 525) where the N-terminal amine group of a tryptophan (W) residue at the first position is cyclized with the side-chain carboxylic acid group of a glutamic acid (E) residue at the ninth position

Codon-optimized DNA encoding the sequences for the ukn22 precursor peptide (peptide No: 525), peptidase (peptide No: 1584), cyclase (peptide No: 2676) and RRE (peptide No: 3975) from Thermobifida fusca are synthesized (Thermo Fisher, Carlsbad, Calif.) and individually cloned into a pZE expression vector (Expressys) behind a maltose binding protein (MBP) sequence to create an N-terminal MBP fusion protein. The resulting plasmids encoding fusion genes for the MBP-ukn22 precursor peptide (peptide No: 525), MBP-peptidase (peptide No: 1584), MBP-cyclase (peptide No: 2676) and MBP-RRE (peptide No: 3975) are driven by a constitutive T7 promoter. The MBP fusion proteins are produced either separately in individual vessels or in combination in one single vessel by introducing DNA plasmid vectors into the vessel containing E. coli BL21 Star(DE3) cell extracts (15 mg/mL total protein) which is pre-mixed with the buffer described above to achieve a total volume of 50 μL. The MBP fusion proteins are then purified using amylose resin (New England BioLabs) according to the manufacturer's recommendation. The cell-free biosynthesis of ukn22 lasso peptide is accomplished by incubating the isolated MBP fusion proteins for 16 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2269 m/z corresponding to ukn22 lasso peptide (WYTAEWGLELIFVFPRFI (SEQ ID NO: 2632) minus H₂O) is observed. The collected lasso peptide sample is further purified by affinity chromatography and/or preparative HPLC, followed by high resolution mass spectrometry and NMR for structural characterization.

### Example 8 Screening of Lariatin Lasso Peptide Against G Protein-Couple Receptors (GPCRs)

Isolated lariatin lasso peptide is lyophilized and reconstituted in 100% DMSO to achieve 10 mM stock. Screening of lariatin lasso peptide against a panel of G protein-couple receptors (GPCRs) follows the manufacturer's recommendation (PathHunter® β-Arrestin eXpress GPCR Assay, Eurofins DiscoverX). The screen is performed at both “agonist” and “antagonist” modes if a known nature ligand is available, and only at “agonist” mode if no known ligand is available. The effect of lariatin lasso peptide on the selected GPCRs is measured by β-Arrestin recruitment using a technology developed by Eurofins DiscoverX called Enzyme Fragment Complementation (EFC) with β-galactosidase (β-Gal) as the functional reporter. PathHunter GPCR cells are expanded from freezer stocks according to the manufacture's procedures. Cells are seeded in a total volume of 20 μL into white walled, 384-well microplates and incubated at 37° C. for the appropriate time prior to testing. For agonist determination, cells are incubated with sample to induce response. Intermediate dilution of sample stocks is performed to generate 5× sample in assay buffer. Five microliters of 5× sample is added to cells and incubated at 37° C. or room temperature for 90 to 180 minutes. Vehicle (DMSO) concentration is 1%. For inverse agonist determination, cells are incubated with sample to induce response. Intermediate dilution of sample stocks is performed to generate 5× sample in assay buffer. Five microliters of 5× sample is added to cells and incubated at 37° C. or room temperature for 3 to 4 hours. Vehicle (DMSO) concentration is 1%. Extended incubation is typically required to observe an inverse agonist response in the PathHunter arrestin assay. For antagonist determination, cells are preincubated with antagonist followed by agonist challenge at the EC80 concentration. Intermediate dilution of sample stocks is performed to generate 5× sample in assay buffer. Five microliters of 5× sample is added to cells and incubated at 37° C. or room temperature for 30 minutes. Vehicle (DMSO) concentration is 1%. Five microliters of 6× EC80 agonist in assay buffer is added to the cells and incubated at 37° C. or room temperature for 90 or 180 minutes. After appropriate compound incubation, assay signal is generated through a single addition of 12.5 μL (50% v/v) of PathHunter Detection reagent cocktail for agonist and inverse agonist assays, followed by a one-hour incubation at room temperature. For some GPCRs that exhibit low basal signal, activity is detected using a high sensitivity detection reagent (PathHunter Flash Kit) to improve assay performance. For these assays an equal volume (25 μL) of detection reagent is added to the wells and incubated for one hour at room temperature. Microplates are read following signal generation with a PerkinElmer Envision™ instrument for chemiluminescent signal detection.

### Example 9 Creation of a Lasso Peptide Library

To create a library of lasso peptides, codon-optimized DNA encoding the sequences described above for capistruin precursor peptide (peptide No: 15), capistruin peptidase (peptide No: 1566), capistruin cyclase (peptide No: 3438), lariatin precursor peptide (peptide No: 162), lariatin peptidase (peptide No: 1368), lariatin cyclase (peptide No: 2406), lariatin RRE (peptide No: 3803), ukn16 precursor peptide (peptide No: 823), ukn16 peptidase (peptide No: 1442), ukn16 cyclase-RRE fusion protein (peptide No: 2504), adanomysin precursor peptide (peptide No: 839), adanomysin cyclase (peptide No: 3128), and adanomysin RRE-peptidase fusion protein (peptide No: 4150) are synthesized (Thermo Fisher, Carlsbad, Calif.) and individually cloned into a pZE expression vector behind a T7 promoter (Expressys). The resulting plasmids encode genes for biosynthesis of capistruin, lariatin, ukn16 and adanomysin with or without a C-terminal affinity tag. Production of the fours lasso peptides in one single vessel is initiated by adding all the plasmids (15 nM each) to the optimized E. coli BL21 Star(DE3) cell extracts, which are pre-mixed with buffer that contains ATP, GTP, TTP, CTP, amino acids, t-RNA, magnesium glutamate, potassium glutamate, potassium phosphate, and other salts, NAD+, NADPH, and glucose to achieve a total volume of 400 μL. The cell-free biosynthesis of the four lasso peptides are accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid fraction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2049 m/z corresponding to capistruin lasso peptide (GTPGFQTPDARVISRFGFN (SEQ ID NO: 2633) minus H₂O), the molecular mass of 2204 m/z corresponding to lariatin lasso peptide (GSQLVYREWVGHSNVIKPGP (SEQ ID NO: 2634) minus H₂O), the molecular mass of 2306 m/z corresponding to ukn16 lasso peptide (GVWFGNYVDVGGAKAPFPWGSN (SEQ ID NO: 2635) minus H₂O), and the molecular mass of 1676 m/z corresponding to adanomysin lasso peptide (GSSTSGTADANSQYYW (SEQ ID NO: 2636) minus H₂O) are observed. The collected lasso peptide sample is further purified by affinity chromatography and/or preparative HPLC, followed by high resolution mass spectrometry and NMR for structural characterization.

### Example 10 Evolution of Lariatin Lasso Peptide Via Site-Saturation Mutagenesis

Codon-optimized DNA encoding the sequences for the lariatin precursor peptide (peptide No: 162), peptidase (peptide No: 1368), cyclase (peptide No: 2406) and RRE (peptide No: 3803) from Rhodococcus jostii are synthesized (Thermo Fisher, Carlsbad, Calif.) and individually cloned into a pZE expression vector behind a T7 promoter (Expressys). The resulting plasmids encoding genes for the lariatin precursor peptide (peptide No: 162), peptidase (peptide No: 1368), cyclase (peptide No: 2406) and RRE (peptide No: 3803) are used with or without a C-terminal affinity tag. To generation a site-saturation library of lariatin lasso peptide variants, each amino acid codon of lanatin core peptide GSQLVYREWVGHSNVIKPGP (SEQ ID NO: 2634) is mutagenized to non-parental amino acid codons with the exception of the glycine (G) residue at the first position and the glutamic acid (E) at the eighth position that are required for cyclization. The site-saturation mutagenesis is performed using QuikChange Lightning Site-Directed Mutagenesis kit (Agilent Technologies, CA) following the manufacturer's recommended protocol. The mutagenic oligonucleotide primers are synthesized (Integrated DNA Technologies, IL) and used either individually to incorporate a non-parental codon into the lanatin core peptide in a single vessel or in combination to incorporate more than one non-parental codons (e.g., NNK) into the lariatin core peptide in a single vessel. To create combinatorial mutation variants of lariatin lasso peptide during a lasso peptide evolution cycle, the mutagenic oligonucleotide primers are synthesized (Integrated DNA Technologies, IL) to simultaneously incorporate more than one codon changes.

Production of a lariatin lasso peptide variant is initiated by adding the plasmids encoding a mutated lanatin precursor peptide (variant of peptide No: 162), lariatin peptidase (peptide No: 1368), lariatin cyclase (peptide No: 2406) and lanatin RRE (peptide No: 3803) (15 nM each) in a single vessel containing the optimized E. coli BL21 Star(DE3) cell extracts, which are pre-mixed with buffer that contains ATP, GTP, TIP, CTP, amino acids, t-RNA, magnesium glutamate, potassium glutamate, potassium phosphate, and other salts, NAD+, NADPH, and glucose to achieve a total volume of 400 μL. The cell-free biosynthesis of a lariatin lasso peptide variant is accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid fraction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass corresponding to the lariatin lasso peptide variant (linear core peptide sequence minus H₂O) is observed. The collected lasso peptide sample is further purified by affinity chromatography and/or preparative HPLC, followed by high resolution mass spectrometry and NMR for structural characterization.

### Example 11

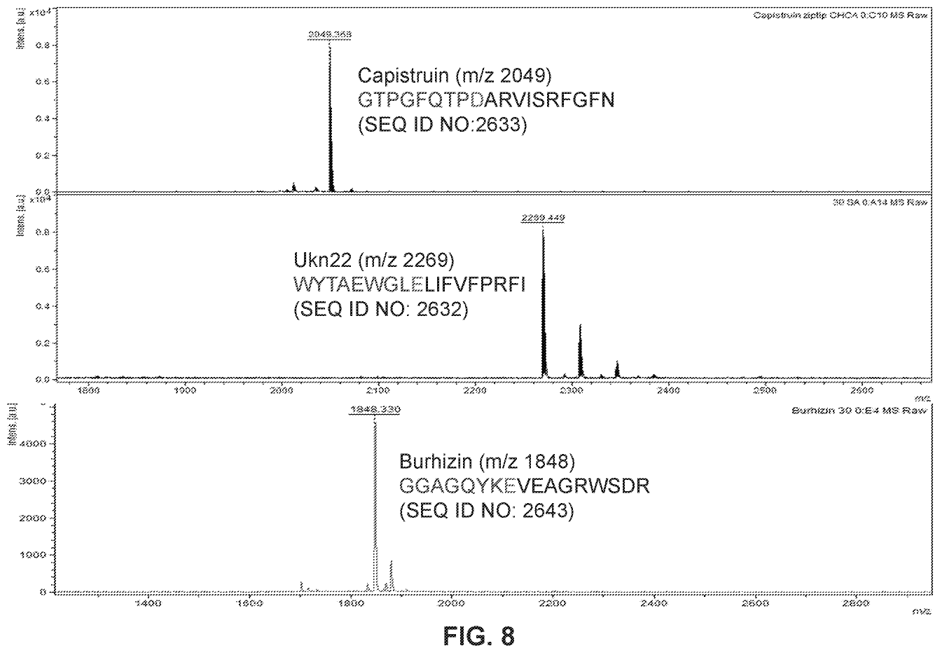

This study demonstrates cell-free biosynthesis of a three-member lasso peptide library in individual vessels. The library members comprised capsitruin (the lasso peptide of peptide No: 15 (SEQ ID NO: 2633)), ukn22 (the lasso peptide of peptide No: 525 (SEQ ID NO: 2632)) and burhizin (the lasso peptide of peptide No: 111) GGAGQYKEVEAGRWSDR (SEQ ID NO: 2643) (FIG. 8). Synthesis of capsitruin (SEQ ID NO: 2633) and burhizin (SEQ ID NO: 2643) was achieved by adding the corresponding BGC DNA sequences into the individual vessels.

The biosynthetic gene cluster (BGC) DNA sequence from Burkholderia thailandensis containing the open reading frames (ORFs) for a capistruin lasso precursor peptide (peptide No: 15), capistruin peptidase (peptide No: 1566) and capistruin cyclase (peptide No: 3438) was cloned into a pET41a plasmid vector. Similarly, the BGC DNA sequence from Burkholderia rhizoxinica containing the ORFs for a burhizin lasso precursor peptide (peptide No: 111), burhizin peptidase (peptide No: 2033) and burhizin cyclase (peptide No: 2722) was cloned into a second pET41a plasmid vector. Following the procedure described in Example 2, the four DNA plasmid vectors for biosynthesis of ukn22 were constructed to produce the MBP-ukn22 precursor peptide (peptide No: 525), MBP-peptidase (peptide No: 1584), MBP-cyclase (peptide No: 2676) and MBP-RRE (peptide No: 3975). The identity of all cloned DNA sequences was verified by Sanger DNA sequencing. High purity DNA plasmid vectors were prepared by Qiagen Plasmid Maxi Kit. Production of these three lasso peptides was initiated in individual vessels by adding the capistruin BGC plasmid vector into the first vessel, the burhizin BGC plasmid vector into the second vessel, and the four ukn22 plasmid vectors into the third vessel. Each of the three vessels contained the optimized E. coli BL21 Star(DE3) cell extracts, which were pre-mixed with buffer that contained ATP, GTP, TTP, CTP, amino acids, t-RNA, magnesium glutamate, potassium glutamate, potassium phosphate, and other salts, NAD+, NADPH, and glucose to achieve a total volume of 40 μL. The concentration of the DNA plasmid vectors was 20 nM for the capistruin BGC plasmid vector in the first vessel, 40 nM for the burhizin BGC plasmid vector in the second vessel and 10 nM each for the four ukn22 plasmid vectors in the third vessel. The cell-free biosynthesis of the lasso peptides was accomplished by incubating the reaction for 18 hours at 25° C. Each reaction sample was subsequently desalted, concentrated and purified with ZipTip® pipette tips (MilliporeSigma ZipTip®) and subjected to MALDI-TOF analysis on a Bruker UltrafleXtreme MALDI TOF/TOF mass spectrometer. The molecular mass corresponding to capsitruin (the linear core peptide of peptide No: 15 (SEQ ID NO: 2633) minus H₂O), ukn22 (the linear core peptide of peptide No: 525 (SEQ ID NO: 2632) minus H₂O) and burhizin (the linear core peptide of peptide No: 111 (SEQ ID NO: 2643) minus H₂O) was observed (FIG. 8).

### Example 12

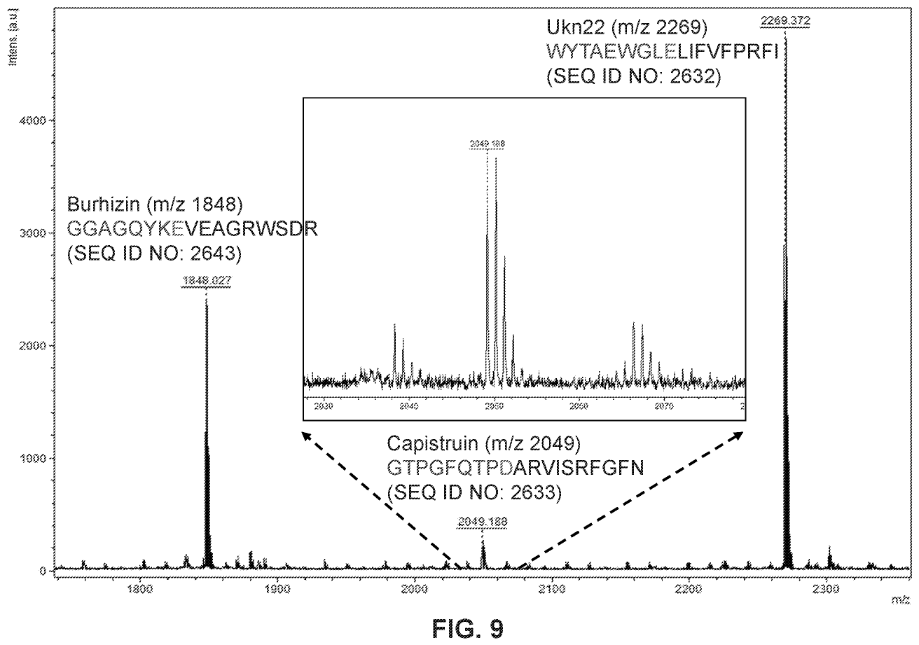

This study demonstrates cell-free biosynthesis of a three-member lasso peptide library in a single vessel. The library members comprised capsitruin (the lasso peptide of peptide No: 15 (SEQ ID NO: 2633)), ukn22 (the lasso peptide of peptide No: 525 (SEQ ID NO: 2632)) and burhizin (the lasso peptide of peptide No: 111 (SEQ ID NO: 2643)) (FIG. 9). Synthesis of capsitruin (SEQ ID NO: 2633) and burhizin (SEQ ID NO: 2643) was achieved by adding the corresponding BGC DNA sequences into the single vessel.

The biosynthetic gene cluster (BGC) DNA sequence from Burkholderia thailandensis containing the open reading frames (ORFs) for a capistruin lasso precursor peptide (peptide No: 15), capistruin peptidase (peptide No: 1566) and capistruin cyclase (peptide No: 3438) was cloned into a pET41a plasmid vector. Similarly, the BGC DNA sequence from Burkholderia rhizoxinica containing the ORFs for a burhizin lasso precursor peptide (peptide No: 111), burhizin peptidase (peptide No: 2033) and burhizin cyclase (peptide No: 2722) was cloned into a second pET41a plasmid vector. Following the procedure described in Example 2, the four DNA plasmid vectors for biosynthesis of ukn22 were constructed to produce the MBP-ukn22 precursor peptide (peptide No: 525), MBP-peptidase (peptide No: 1584), MBP-cyclase (peptide No: 2676) and MBP-RRE (peptide No: 3975). The identity of all cloned DNA sequences was verified by Sanger DNA sequencing. High purity DNA plasmid vectors were prepared by Qiagen Plasmid Maxi Kit. Production of these three lasso peptides was initiated in a single vessel by adding the capistruin and burhizin BGC plasmid vectors and the four ukn22 plasmid vectors into the vessel. The single vessel contained the optimized E. coli BL21 Star(DE3) cell extracts, which were pre-mixed with buffer that contained ATP, GTP, TIP, CTP, amino acids, t-RNA, magnesium glutamate, potassium glutamate, potassium phosphate, and other salts, NAD+, NADPH, and glucose to achieve a total volume of 40 μL. The concentration of the DNA plasmid vectors in the single vessel was 20 nM for the capistruin BGC plasmid vector, 10 nM for the burhizin BGC plasmid vector and 5 nM each for the four ukn22 plasmid vectors. The cell-free biosynthesis of the lasso peptides was accomplished by incubating the reaction for 18 hours at 25° C. The reaction sample was subsequently desalted, concentrated and purified with ZipTip® pipette tips (MilliporeSigma ZipTip®) and subjected to MALDI-TOF analysis on a Bruker UltrafleXtreme MALDI TOF/TOF mass spectrometer. The molecular mass corresponding to capsitruin (the linear core peptide of peptide No: 15 (SEQ ID NO: 2633) minus H₂O), ukn22 (the linear core peptide of peptide No: 525 (SEQ ID NO: 2632) minus H₂O) and burhizin (the linear core peptide of peptide No: 111 (SEQ ID NO: 2643) minus H₂O) was observed (FIG. 9).

### Example 13

This study demonstrates cell-free biosynthesis of a six-member lasso peptide library in individual vessels. The library members comprised ukn22 lasso peptide (the lasso peptide of peptide No: 525 (SEQ ID NO: 2632)) and the five variants of ukn22 lasso peptide, including ukn22 W1Y (SEQ ID NO: 2638), ukn22 W1F (SEQ ID NO: 2639), ukn22 W1H (SEQ ID NO: 2640), ukn22 W1L (SEQ ID NO: 2641) and ukn22 W1A (SEQ ID NO: 2642) as listed in Table X3.

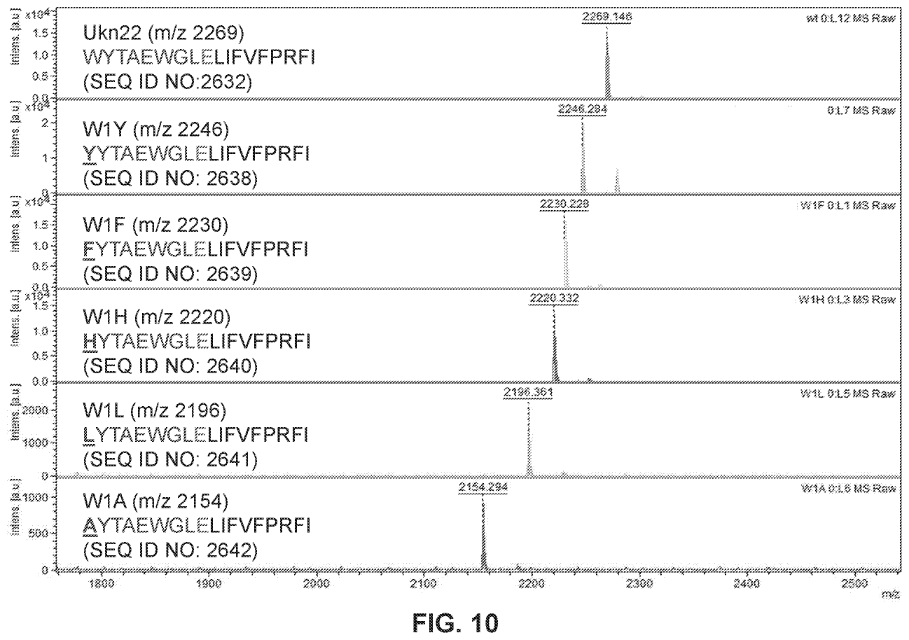

Construction of the six-member lasso peptide library followed the method described in Example 2. The plasmid vectors encoding the MBP-ukn22 precursor peptide (peptide No: 525) was mutagenized to generate five ukn22 precursor peptide variants (variants of peptide No: 525). Each of the five ukn22 precursor peptide variants comprised of the ukn22 leader peptide sequence MEKKKYTAPQLAKVGEFKEATG (SEQ ID NO: 2637) (the leader sequence of peptide No: 525) and a mutated ukn22 core peptide sequence WYTAEWGLELIFVFPRFI (SEQ ID NO: 2632) (the core sequence of peptide No: 525). Following the DNA mutagenesis procedure described in Example 10, the first Tryptophan residue (W) of the ukn22 core peptide sequence was changed to Tyrosin (Y), Phenylalanine (F), Histidine (H), Leucine (L) or Alanine (A). The resulting ukn22 precursor peptide variants were designated as ukn22 W1Y, ukn22 W1F, ukn22 W1H, ukn22 W1L and ukn22 W1A. The linear core sequence of each variant was listed in Table X3. Production of these six lasso peptides was initiated in six separate vessels by sequentially adding one precursor peptide plasmid vector per vessel for ukn22, ukn22 W1Y, ukn22 W1F, ukn22 W1H, ukn22 W1L and ukn22 W1A at the concentration of 10 nM per plasmid vector. Each of the six vessels contained the optimized E. coli BL21 Star(DE3) cell extracts, which were pre-mixed with buffer that contained ATP, GTP, TIP, CTP, amino acids, t-RNA, magnesium glutamate, potassium glutamate, potassium phosphate, and other salts, NAD+, NADPH, and glucose to achieve a total volume of 40 μL. The plasmid vectors encoding MBP-peptidase (peptide No: 1584), MBP-cyclase (peptide No: 2676) and MBP-RRE (peptide No: 3975) were subsequently added into each vessel at the concentration of 10 nM each. The cell-free biosynthesis of the lasso peptides was accomplished by incubating the reaction for 18 hours at 25° C. Each reaction sample was subsequently desalted, concentrated and purified with ZipTip® pipette tips (MilliporeSigma ZipTip®) and subjected to MALDI-TOF analysis on a Bruker UltrafleXtreme MALDI TOF/TOF mass spectrometer. The molecular mass corresponding to the lasso peptide of ukn22 (SEQ ID NO: 2632 minus H₂O), ukn22 W1Y (SEQ ID NO: 2638 minus H₂O), ukn22 W1F (SEQ ID NO: 2639 minus H₂O), ukn22 W1H (SEQ ID NO: 2640 minus H₂O), ukn22 W1L (SEQ ID NO: 2641 minus H₂O) and ukn22 W1A (SEQ ID NO: 2642 minus H₂O) was observed (FIG. 10)

### Example 14

This study demonstrates cell-free biosynthesis of a six-member lasso peptide library in a single vessel. The library members comprised ukn22 lasso peptide (the lasso peptide of peptide No: 525 (SEQ ID NO: 2632)) and the five variants of ukn22 lasso peptide, including ukn22 W1Y (SEQ ID NO: 2638), ukn22 W1F (SEQ ID NO: 2639), ukn22 W1H (SEQ ID NO: 2640), ukn22 W1L (SEQ ID NO: 2641) and ukn22 W1A (SEQ ID NO: 2642) as listed in Table X3

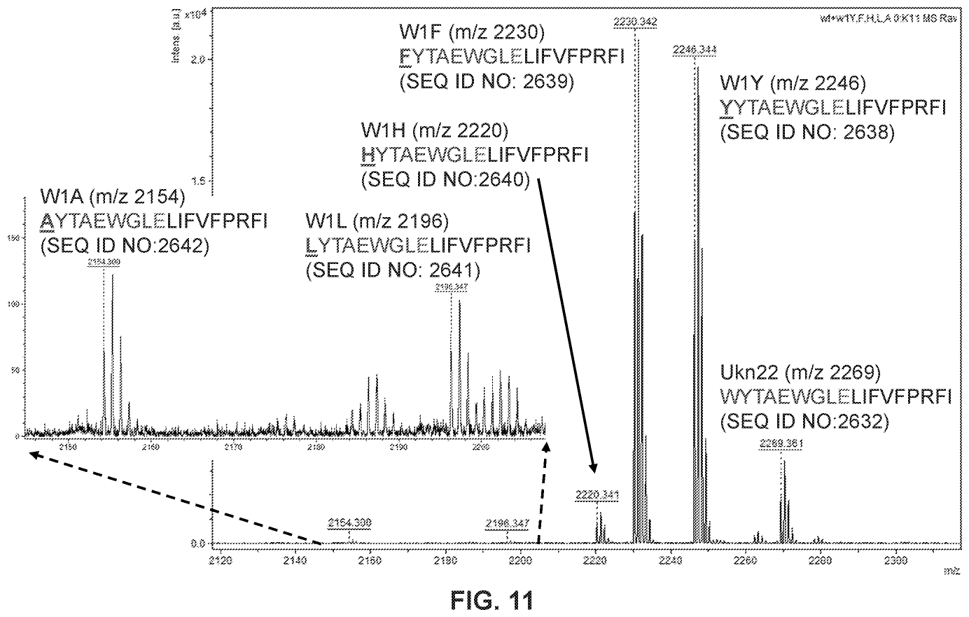

Construction of the six-member lasso peptide library followed the method described in Example 13. Production of these six lasso peptides was initiated in a single vessel by simultaneously adding the six precursor peptide plasmids for ukn22, ukn22 W1Y, ukn22 W1F, ukn22 W1H, ukn22 W1L and ukn22 W1A at the concentration of 10 nM per plasmid vector. The single vessel contained the optimized E. coli BL21 Star(DE3) cell extracts, which were pre-mixed with buffer that contained ATP, GTP, TTP, CTP, amino acids, t-RNA, magnesium glutamate, potassium glutamate, potassium phosphate, and other salts, NAD+, NADPH, and glucose to achieve a total volume of 40 μL. The plasmid vectors encoding MBP-peptidase (peptide No: 1584), MBP-cyclase (peptide No: 2676) and MBP-RRE (peptide No: 3975) were subsequently added into the vessel at the concentration of 10 nM each. The cell-free biosynthesis of the lasso peptides was accomplished by incubating the reaction for 18 hours at 25° C. The reaction sample was subsequently desalted, concentrated and purified with ZipTip® pipette tips (MilliporeSigma ZipTip®) and subjected to MALDI-TOF analysis on a Bruker UltrafleXtreme MALDI TOF/TOF mass spectrometer. The molecular mass corresponding to the lasso peptide of ukn22 (SEQ ID NO: 2632 minus H₂O), ukn22 W1Y (SEQ ID NO: 2638 minus H₂O), ukn22 W1F (SEQ ID NO: 2639 minus H₂O), ukn22 W1H (SEQ ID NO: 2640 minus H₂O), ukn22 W1L (SEQ ID NO: 2641 minus H₂O) and ukn22 W1A (SEQ ID NO: 2642 minus H₂O) was observed (FIG. 11).

### Example 15

This study demonstrates cell-free biosynthesis of cellulonodin lasso peptide WIQGKWGLEIYLIFPRYL (SEQ ID: 2652) where the N-terminal amine group of a tryptophan (W) residue at the first position was cyclized with the side-chain carboxylic acid group of a glutamic acid (E) residue at the ninth position.

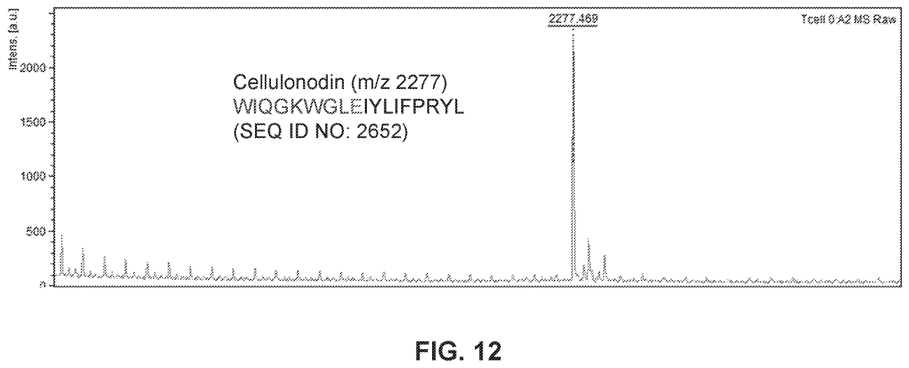

The biosynthetic gene cluster (BGC) DNA sequence from Thermobifida cellulosilytica TB100 containing the open reading fame (ORF) (SEQ ID NO: 2644) for a cellulonodin lasso precursor peptide (SEQ ID No: 2645), the ORF (SEQ ID NO: 2646) for cellulonodin peptidase (SEQ ID No: 2647), the ORF (SEQ ID NO: 2648) for cellulonodin cyclase (SEQ ID No: 2649), and the ORF (SEQ ID NO: 2650) for cellulonodin RRE (SEQ ID NO: 2651) were cloned into a pET41a plasmid vector. The identity of the cloned DNA sequences was verified by Sanger DNA sequencing. High purity DNA plasmid vector was prepared by Qiagen Plasmid Maxi Kit. Production of cellulonodin lasso peptide was initiated by adding the cellulonodin BGC plasmid vectors into a single vessel. The vessel contained the optimized E. coli BL21 Star(DE3) cell extracts, which were pre-mixed with buffer that contained ATP, GTP, TTP, CTP, amino acids, t-RNA, magnesium glutamate, potassium glutamate, potassium phosphate, and other salts, NAD+, NADPH, and glucose to achieve a total volume of 20 μL. The concentration of the cellulonodin BGC plasmid vector in the vessel was 40 nM. The cell-free biosynthesis of the lasso peptides was accomplished by incubating the reaction for 18 hours at 25° C. The reaction sample was subsequently desalted, concentrated and purified with ZipTip® pipette tips (MilliporeSigma ZipTip®) and subjected to MALDI-TOF analysis on a Bruker UltrafleXtreme MALDI TOF/TOF mass spectrometer. The molecular mass corresponding to cellulonodin (SEQ ID NO: 2652) minus H₂O) was observed (FIG. 12).

### 7. SEQUENCES

Various exemplary amino acid and nucleic acid sequences are disclosed in this application, a summary of which are provided in the Table 1. Additionally, Table 2 lists exemplary combinations of various components that can be used in connection with the present methods and systems. Table 3 lists examples of lasso peptidase. Table 4 lists examples of lasso cyclase. Table 5 lists examples of RREs.

TABLE 1
Summary Table

| Class | Description | Peptide No: # |
|---|---|---|
| A | Precursors | 1-1315 |
| B | Peptidase | 1316-2336 |
| C* | Cyclase | 2337-3761 |
| E** | RRE | 3762-4593 |
| CE | cyclase-RRE fusion | 2504 |
| CB | cyclase-peptidase fusion | 2903 |
| CE | cyclase-RRE fusion | 3608 |
| EB | RRE-peptidase fusion | 3768 |
| EB | RRE-peptidase fusion | 3770 |
| EB | RRE-peptidase fusion | 3793 |
| EB | RRE-peptidase fusion | 3811 |
| EB | RRE-peptidase fusion | 3818 |
| EB | RRE-peptidase fusion | 3851 |
| EB | RRE-peptidase fusion | 3855 |
| EB | RRE-peptidase fusion | 3887 |
| EB | RRE-peptidase fusion | 4004 |
| EB | RRE-peptidase fusion | 4018 |
| EB | RRE-peptidase fusion | 4045 |
| EB | RRE-peptidase fusion | 4076 |
| EB | RRE-peptidase fusion | 4132 |
| EB | RRE-peptidase fusion | 4150 |
| EB | RRE-peptidase fusion | 4167 |
| EB | RRE-peptidase fusion | 4168 |
| EB | RRE-peptidase fusion | 4225 |
| EB | RRE-peptidase fusion | 4262 |
| EB | RRE-peptidase fusion | 4379 |
| EB | RRE-peptidase fusion | 4414 |
| EB | RRE-peptidase fusion | 4499 |
| EB | RRE-peptidase fusion | 4504 |
| EB | RRE-peptidase fusion | 4507 |
| EB | RRE-peptidase fusion | 4512 |
| EB | RRE-peptidase fusion | 4517 |
| EB | RRE-peptidase fusion | 4518 |
| EB | RRE-peptidase fusion | 4529 |
| EB | RRE-peptidase fusion | 4532 |
| EB | RRE-peptidase fusion | 4542 |
| EB | RRE-peptidase fusion | 4559 |
| EB | RRE-peptidase fusion | 4561 |
| EB | RRE-peptidase fusion | 4562 |

*including CE and CB fusion sequences
**Including EB fusion sequences

TABLE 2
Exemplary Combinations of (i) Lasso Precursor Peptide; (ii) Lasso
Peptidase; (iii) Lasso Cyclase; (iv) RRE; (v) Peptidase Fusion; and/or
(vi) Cyclase Fusion

| Peptide No: #; GI#; | | | | | |
|---|---|---|---|---|---|
| Accession#; Nucleic | | | | | |
| Acid SEQ ID NO: #; | | | | | |
| Amino Acid SEQ ID | Peptidase | Cyclase | RRE | CE | EB |
| NO: #; Junction | Peptide | Peptide | Peptide | Peptide | Peptide |
| Position | No: # | No: # | No: # | No: # | No: # |
| 1; 167643973; | 1598 | 3360 | n/a | n/a | n/a |
| NC_010338.1; 1; 2; | | | | | |
| 22/23 | | | | | |
| 2; 167643973; | 1598 | 3360 | n/a | n/a | n/a |
| NC_010338.1; 3; 4; | | | | | |
| 21/22 | | | | | |
| 3; 167643973; | 1324 | 2349 | n/a | n/a | n/a |
| NC_010338.1; 5; 6; | | | | | |
| 21/22 | | | | | |
| 4; 167643973; | 1324 | 2349 | n/a | n/a | n/a |
| NC_010338.1; 7; 8; | | | | | |
| 22/23 | | | | | |
| 5; 737103862; | 1943 | 3191 | n/a | n/a | n/a |
| NZ_JQJP01000023.1; 9; | | | | | |
| 10; 21/22 | | | | | |
| 6; 737089868; | 1943 | 3191 | n/a | n/a | n/a |
| NZ_JQJN01000025.1; | | | | | |
| 11; 12; 21/22 | | | | | |
| 7; 737089868; | 1942 | 3190 | n/a | n/a | n/a |
| NZ_JQJN01000025.1; | | | | | |
| 13; 14; 21/22 | | | | | |
| 8; 737089868; | 1942 | 3190 | n/a | n/a | n/a |
| NZ_JQJN01000025.1; | | | | | |
| 15; 16; 21/22 | | | | | |
| 9; 930490730 | 2056 | 3614 | 4407 | n/a | n/a |
| NZ_LJCU01000014.1; | | | | | |
| 17; 18; 13/14 | | | | | |
| 10; 930490730 | 2279 | 3681 | 4541 | n/a | n/a |
| NZ_LJCU01000014.1; | | | | | |
| 19; 20; 13/14 | | | | | |
| 11; 657284919; | 1438 | 2500 | 3861 | n/a | n/a |
| JJMG01000143.1; 21; | | | | | |
| 22; 21/22 | | | | | |
| 12; 657284919; | 2114 | 3635 | 4459 | n/a | n/a |
| JJMG01000143.1; 23; | | | | | |
| 24; 21/22 | | | | | |
| 13; 657284919; | 1988 | 3570 | 4347 | n/a | n/a |
| JJMG01000143.1; 25; | | | | | |
| 26; 21/22 | | | | | |
| 14; 663380895; | n/a | 3091 | 4259 | n/a | n/a |
| NZ_JNZW01000001.1; | | | | | |
| 27; 28; 21/22 | | | | | |
| 15; 485035557; | 1566 | 3438 | n/a | n/a | n/a |
| NZ_AECN01000315.1; | | | | | |
| 29; 30; 28/29 | | | | | |
| 16; 485035557; | 1566 | 2971 | n/a | n/a | n/a |
| NZ_AECN01000315.1; | | | | | |
| 31; 32; 28/29 | | | | | |
| 17; 485035557; | 1565 | 2981 | n/a | n/a | n/a |
| NZ_AECN01000315.1; | | | | | |
| 33; 34; 28/29 | | | | | |
| 18; 485035557; | 1565 | 2970 | n/a | n/a | n/a |
| NZ_AECN01000315.1; | | | | | |
| 35; 36; 28/29 | | | | | |
| 19; 485035557; | 1318 | 2339 | n/a | n/a | n/a |
| NZ_AECN01000315.1; | | | | | |
| 37; 38; 28/29 | | | | | |
| 20; 485035557; | 1644 | 2772 | n/a | n/a | n/a |
| NZ_AECN01000315.1; | | | | | |
| 39; 40; 28/29 | | | | | |
| 21; 485035557; | 1533 | 3393 | n/a | n/a | n/a |
| NZ_AECN01000315.1; | | | | | |
| 41; 42; 28/29 | | | | | |
| 22; 485035557; | 1399 | 2451 | n/a | n/a | n/a |
| NZ_AECN01000315.1; | | | | | |
| 43; 44; 28/29 | | | | | |
| 23; 149147045; | 1571 | 3436 | n/a | n/a | n/a |
| NZ_ABBG01000168.1; | | | | | |
| 45; 46; 28/29 | | | | | |
| 24; 67639376; | 1525 | 3349 | n/a | n/a | n/a |
| NZ_AAHO01000116.1; | | | | | |
| 47; 48; 28/29 | | | | | |
| 25; 149147045; | 1570 | 3300 | n/a | n/a | n/a |
| NZ_ABBG01000168.1; | | | | | |
| 49; 50; 28/29 | | | | | |
| 26; 67639376; | 1523 | 2613 | n/a | n/a | n/a |
| NZ_AAHO01000116.1; | | | | | |
| 51; 52; 28/29 | | | | | |
| 27; 67639376; | 1525 | 3292 | n/a | n/a | n/a |
| NZ_AAHO01000116.1; | | | | | |
| 53; 54; 28/29 | | | | | |
| 28; 67639376; | 1523 | 3283 | n/a | n/a | n/a |
| NZ_AAHO01000116.1; | | | | | |
| 55; 56; 28/29 | | | | | |
| 29; 67639376; | 1526 | 3287 | n/a | n/a | n/a |
| NZ_AAHO01000116.1; | | | | | |
| 57; 58; 28/29 | | | | | |
| 30; 67639376; | 1525 | 2612 | n/a | n/a | n/a |
| NZ_AAHO01000116.1; | | | | | |
| 59; 60; 28/29 | | | | | |
| 31; 67639376; | 1525 | 3280 | n/a | n/a | n/a |
| NZ_AAHO01000116.1; | | | | | |
| 61; 62; 28/29 | | | | | |
| 32; 67639376; | 1526 | 3350 | n/a | n/a | n/a |
| NZ_AAHO01000116.1; | | | | | |
| 63; 64; 28/29 | | | | | |
| 33; 67639376; | 1525 | 3295 | n/a | n/a | n/a |
| NZ_AAHO01000116.1; | | | | | |
| 65; 66; 28/29 | | | | | |
| 34; 67639376; | 1525 | 3285 | n/a | n/a | n/a |
| NZ_AAHO01000116.1; | | | | | |
| 67; 68; 28/29 | | | | | |
| 35; 67639376; | 1523 | 3298 | n/a | n/a | n/a |
| NZ_AAHO01000116.1; | | | | | |
| 69; 70; 28/29 | | | | | |
| 36; 67639376; | 1526 | 3296 | n/a | n/a | n/a |
| NZ_AAHO01000116.1; | | | | | |
| 71; 72; 28/29 | | | | | |
| 37; 67639376; | 1525 | 3544 | n/a | n/a | n/a |
| NZ_AAHO01000116.1; | | | | | |
| 73; 74; 28/29 | | | | | |
| 38; 67639376; | 1526 | 3545 | n/a | n/a | n/a |
| NZ_AAHO01000116.1; | | | | | |
| 75; 76; 28/29 | | | | | |
| 39; 67639376; | 1524 | 2611 | n/a | n/a | n/a |
| NZ_AAHO01000116.1; | | | | | |
| 77; 78; 28/29 | | | | | |
| 40; 67639376; | 1523 | 2614 | n/a | n/a | n/a |
| NZ_AAHO01000116.1; | | | | | |
| 79; 80; 28/29 | | | | | |
| 41; 67639376; | 1526 | 3352 | n/a | n/a | n/a |
| NZ_AAHO01000116.1; | | | | | |
| 81; 82; 28/29 | | | | | |
| 42; 67639376; | 1525 | 3297 | n/a | n/a | n/a |
| NZ_AAHO01000116.1; | | | | | |
| 83; 84; 28/29 | | | | | |
| 43; 67639376; | 1525 | 3290 | n/a | n/a | n/a |
| NZ_AAHO01000116.1; | | | | | |
| 85; 86; 28/29 | | | | | |
| 44; 67639376; | 1396 | 2448 | n/a | n/a | n/a |
| NZ_AAHO01000116.1; | | | | | |
| 87; 88; 28/29 | | | | | |
| 45; 67639376; | 1523 | 3409 | n/a | n/a | n/a |
| NZ_AAHO01000116.1; | | | | | |
| 89; 90; 28/29 | | | | | |
| 46; 67639376; | 1525 | 3293 | n/a | n/a | n/a |
| NZ_AAHO01000116.1; | | | | | |
| 91; 92; 28/29 | | | | | |
| 47; 67639376; | 1526 | 3392 | n/a | n/a | n/a |
| NZ_AAHO01000116.1; | | | | | |
| 93; 94; 28/29 | | | | | |
| 48; 67639376; | 1525 | 3291 | n/a | n/a | n/a |
| NZ_AAHO01000116.1; | | | | | |
| 95; 96; 28/29 | | | | | |
| 49; 67639376; | 1525 | 2951 | n/a | n/a | n/a |
| NZ_AAHO01000116.1; | | | | | |
| 97; 98; 28/29 | | | | | |
| 50; 67639376; | 1525 | 3440 | n/a | n/a | n/a |
| NZ_AAHO01000116.1; | | | | | |
| 99; 100; 28/29 | | | | | |
| 51; 67639376; | 1997 | 3282 | n/a | n/a | n/a |
| NZ_AAHO01000116.1; | | | | | |
| 101; 102; 28/29 | | | | | |
| 52; 67639376; | 1526 | 2615 | n/a | n/a | n/a |
| NZ_AAHO01000116.1; | | | | | |
| 103; 104; 28/29 | | | | | |
| 53; 67639376; | 1395 | 2447 | n/a | n/a | n/a |
| NZ_AAHO01000116.1; | | | | | |
| 105; 106; 28/29 | | | | | |
| 54; 67639376; | 1523 | 2610 | n/a | n/a | n/a |
| NZ_AAHO01000116.1; | | | | | |
| 107; 108; 28/29 | | | | | |
| 55; 67639376; | 1523 | 3437 | n/a | n/a | n/a |
| NZ_AAHO01000116.1; | | | | | |
| 109; 110; 28/29 | | | | | |
| 56; 67639376; | 1526 | 3289 | n/a | n/a | n/a |
| NZ_AAHO01000116.1; | | | | | |
| 111; 112;28/29 | | | | | |
| 57; 67639376; | 1523 | 3351 | n/a | n/a | n/a |
| NZ_AAHO01000116.1; | | | | | |
| 113; 114; 28/29 | | | | | |
| 58; 67639376; | 1525 | 3294 | n/a | n/a | n/a |
| NZ_AAHO01000116.1; | | | | | |
| 115; 116; 28/29 | | | | | |
| 59; 67639376; | 1526 | 3281 | n/a | n/a | n/a |
| NZ_AAHO01000116.1; | | | | | |
| 117; 118; 28/29 | | | | | |
| 60; 67639376; | 1317 | 2338 | n/a | n/a | n/a |
| NZ_AAHO01000116.1; | | | | | |
| 119; 120; 28/29 | | | | | |
| 61; 67639376; | 1525 | 3286 | n/a | n/a | n/a |
| NZ_AAHO01000116.1; | | | | | |
| 121; 122; 28/29 | | | | | |
| 62; 67639376; | 1526 | 2690 | n/a | n/a | n/a |
| NZ_AAHO01000116.1; | | | | | |
| 123; 124; 28/29 | | | | | |
| 63; 67639376; | 1447 | 2509 | n/a | n/a | n/a |
| NZ_AAHO01000116.1; | | | | | |
| 125; 126; 28/29 | | | | | |
| 64; 67639376; | 1404 | 2458 | n/a | n/a | n/a |
| NZ_AAHO01000116.1; | | | | | |
| 127; 128; 28/29 | | | | | |
| 65; 67639376; | 1526 | 3284 | n/a | n/a | n/a |
| NZ_AAHO01000116.1; | | | | | |
| 129; 130; 28/29 | | | | | |
| 66; 67639376; | n/a | 2511 | n/a | n/a | n/a |
| NZ_AAHO01000116.1; | | | | | |
| 131; 132; 28/29 | | | | | |
| 67; 67639376; | 1523 | 3383 | n/a | n/a | n/a |
| NZ_AAHO01000116.1; | | | | | |
| 133; 134; 28/29 | | | | | |
| 68; 740958729; | 1998 | 3288 | n/a | n/a | n/a |
| NZ_JPWT01000001.1; | | | | | |
| 135; 136;28/29 | | | | | |
| 69; 485035557; | 1348 | 2380 | n/a | n/a | n/a |
| NZ_AECN01000315.1; | | | | | |
| 137; 138; 28/29 | | | | | |
| 70; 67639376; | 1520 | 2606 | n/a | n/a | n/a |
| NZ_AAHO01000116.1; | | | | | |
| 139; 140; 28/29 | | | | | |
| 71; 149147045; | 1571 | 2982 | n/a | n/a | n/a |
| NZ_ABBG01000168.1; | | | | | |
| 141; 142; 28/29 | | | | | |
| 72; 149147045; | 1570 | 3299 | n/a | n/a | n/a |
| NZ_ABBG01000168.1; | | | | | |
| 143; 144; 28/29 | | | | | |
| 73; 657295264; | n/a | 3465 | 4235 | n/a | n/a |
| NZ_AZSD01000040.1; | | | | | |
| 145; 146; 25/26 | | | | | |
| 74; 754788309; | 1695 | 2846 | 4184 | n/a | n/a |
| NZ_BBNO01000002.1; | | | | | |
| 147; 148; 29/30 | | | | | |
| 75; 928897585; | 2094 | 3458 | 4440 | n/a | n/a |
| NZ_LGKG01000196.1; | | | | | |
| 149; 150; 29/30 | | | | | |
| 76; 928897585; | 2271 | 3671 | 4537 | n/a | n/a |
| NZ_LGKG01000196.1; | | | | | |
| 151; 152; 29/30 | | | | | |
| 77; 754788309; | 2039 | 3370 | 4393 | n/a | n/a |
| NZ_BBNO01000002.1; | | | | | |
| 153; 154; 29/30 | | | | | |
| 78; 739918964; | 1901 | 3267 | 4494 | n/a | n/a |
| NZ_JJOH01000097.1; | | | | | |
| 155; 156; 29/30 | | | | | |
| 79; 928897585; | 1354 | 2386 | 3791 | n/a | n/a |
| NZ_LGKG01000196.1; | | | | | |
| 157; 158; 29/30 | | | | | |
| 80; 374982757; | 2058 | 3397 | 4029 | n/a | n/a |
| NC_016582.1; 159; 160; | | | | | |
| 13/14 | | | | | |
| 81; 374982757; | 2058 | 3397 | 4029 | n/a | n/a |
| NC_016582.1; 161; 162; | | | | | |
| 28/29 | | | | | |
| 82; 739918964; | 1901 | 3583 | 4295 | n/a | n/a |
| NZ_JJOH01000097.1; | | | | | |
| 163; 164; 29/30 | | | | | |
| 83; 852460626; | 1357 | 2392 | 3794 | n/a | n/a |
| CP011799.1; 165; 166; | | | | | |
| 29/30 | | | | | |
| 84; 514918665; | 1661 | 2797 | 4073 | n/a | n/a |
| NZ_AOPZ01000109.1; | | | | | |
| 167; 168; 32/33 | | | | | |
| 85; 396995461; | 2024 | 3338 | 3939 | n/a | n/a |
| AJGV01000085.1; 169; | | | | | |
| 170; 28/29 | | | | | |
| 86; 739830131; | n/a | 3259 | 4351 | n/a | n/a |
| NZ_JOJE01000039.1; | | | | | |
| 171; 172; 32/33 | | | | | |
| 87; 396995461; | 1400 | 2452 | 3833 | n/a | n/a |
| AJGV01000085.1; 173; | | | | | |
| 174; 28/29 | | | | | |
| 88; 374982757; | 1332 | 2357 | 3767 | n/a | 3768 |
| NC_016582.1; 175; 176; | | | | | |
| 13/14 | | | | | |
| 89; 374982757; | 1332 | 2357 | 3767 | n/a | 3768 |
| NC_016582.1; 177; 178; | | | | | |
| 28/29 | | | | | |
| 90; 664481891; | 2144 | 3121 | 4289 | n/a | n/a |
| NZ_JOJI01000011.1; | | | | | |
| 179; 180; 27/28 | | | | | |
| 91; 663732121; | n/a | 3094 | 4498 | n/a | n/a |
| NZ_JNZQ01000012.1; | | | | | |
| 181; 182; 22/23 | | | | | |
| 92; 742921760; | 1492 | 2571 | n/a | n/a | n/a |
| NZ_JWKL01000093.1; | | | | | |
| 183; 184; 37/38 | | | | | |
| 93; 742921760; | 1492 | 3303 | n/a | n/a | n/a |
| NZ_JWKL01000093.1; | | | | | |
| 185; 186; 37/38 | | | | | |
| 94; 389809081; | 2150 | 3328 | n/a | n/a | n/a |
| NZ_AJXW01000057.1; | | | | | |
| 187; 188; 26/27 | | | | | |
| 95; 389809081; | 1398 | 2450 | n/a | n/a | n/a |
| NZ_AJXW01000057.1; | | | | | |
| 189; 190; 26/27 | | | | | |
| 96; 655566937; | 1830 | 3056 | n/a | n/a | n/a |
| NZ_JAES01000046.1; | | | | | |
| 191; 192; 26/27 | | | | | |
| 97; 749673329; | 2020 | 3333 | 4374 | n/a | n/a |
| NZ_JROO01000009.1; | | | | | |
| 193; 194; 20/21 | | | | | |
| 98; 755108320; | 2046 | 3378 | 4399 | n/a | n/a |
| NZ_BBPN01000056.1; | | | | | |
| 195; 196; 16/17 | | | | | |
| 99; 755108320; | 2049 | 3380 | 4402 | n/a | n/a |
| NZ_BBPN01000056.1; | | | | | |
| 197; 198; 16/17 | | | | | |
| 100; 755077919; | 2047 | 3612 | 4400 | n/a | n/a |
| NZ_BBPQ01000048.1; | | | | | |
| 199; 200; 16/17 | | | | | |
| 101; 755077919; | 2048 | 3613 | 4401 | n/a | n/a |
| NZ_BBPQ01000048.1; | | | | | |
| 201; 202; 16/17 | | | | | |
| 102; 167643973; | 2136 | 2697 | n/a | n/a | n/a |
| NC_010338.1; 203; 204; | | | | | |
| 19/20 | | | | | |
| 103; 167643973; | 2136 | 2697 | n/a | n/a | n/a |
| NC_010338.1; 205; 206; | | | | | |
| 19/20 | | | | | |
| 104; 646523831; | 1607 | 2708 | n/a | n/a | n/a |
| NZ_BATN01000047.1; | | | | | |
| 207; 208; 18/19 | | | | | |
| 105; 646523831; | 2231 | 3420 | n/a | n/a | n/a |
| NZ_BATN01000047.1; | | | | | |
| 209; 210; 18/19 | | | | | |
| 106; 739598481; | 2190 | 3237 | n/a | n/a | n/a |
| NZ_JFHR01000062.1; | | | | | |
| 211; 212; 18/19 | | | | | |
| 107; 739598481; | 2190 | 3237 | n/a | n/a | n/a |
| NZ_JFHR01000062.1; | | | | | |
| 213; 214; 18/19 | | | | | |
| 108; 484272664; | 2203 | 3239 | n/a | n/a | n/a |
| NZ_AKIB01000015.1; | | | | | |
| 215; 216; 18/19 | | | | | |
| 109; 484272664; | 1666 | 2805 | n/a | n/a | n/a |
| NZ_AKIB01000015.1; | | | | | |
| 217; 218; 18/19 | | | | | |
| 110; 646523831; | 2241 | 2972 | n/a | n/a | n/a |
| NZ_BATN01000047.1; | | | | | |
| 219; 220; 18/19 | | | | | |
| 111; 312794749; | 2033 | 2722 | n/a | n/a | n/a |
| NC_014722.1; 221; 222; | | | | | |
| 10/11 | | | | | |
| 112; 312794749; | n/a | 2721 | n/a | n/a | n/a |
| NC_014722.1; 223; 224; | | | | | |
| 25/26 | | | | | |
| 113; 652527059; | n/a | 3434 | n/a | n/a | n/a |
| NZ_KE384226.1; 225; | | | | | |
| 226; 27/28 | | | | | |
| 114; 652527059; | n/a | 3007 | n/a | n/a | n/a |
| NZ_KE384226.1; 227; | | | | | |
| 228; 27/28 | | | | | |
| 115; 652527059; | 1790 | 3006 | n/a | n/a | n/a |
| NZ_KE384226.1; 229; | | | | | |
| 230; 28/29 | | | | | |
| 116; 652527059; | 1790 | 3006 | n/a | n/a | n/a |
| NZ_KE384226.1; 231; | | | | | |
| 232; 29/30 | | | | | |
| 117; 652527059; | 1790 | 3006 | n/a | n/a | n/a |
| NZ_KE384226.1; 233; | | | | | |
| 234; 28/29 | | | | | |
| 118; 483624586; | n/a | 2883 | n/a | n/a | n/a |
| NZ_KB889561.1; 235; | | | | | |
| 236; 23/24 | | | | | |
| 119; 221717172; | 1425 | 2481 | 3856 | n/a | n/a |
| DS999644.1; 237; 238; | | | | | |
| 27/28 | | | | | |
| 120; 221717172; | 1569 | 3148 | 3935 | n/a | n/a |
| DS999644.1; 239; 240; | | | | | |
| 27/28 | | | | | |
| 121; 221717172; | 1917 | 3526 | 3935 | n/a | n/a |
| DS999644.1; 241; 242; | | | | | |
| 27/28 | | | | | |
| 122; 221717172; | 1918 | 3536 | 3935 | n/a | n/a |
| DS999644.1; 243; 244; | | | | | |
| 27/28 | | | | | |
| 123; 664184565; | 1443 | 2505 | 3864 | n/a | n/a |
| NZ_JOGA01000019.1; | | | | | |
| 245; 246; 27/28 | | | | | |
| 124; 664184565; | 1919 | 3151 | 4305 | n/a | n/a |
| NZ_JOGA01000019.1; | | | | | |
| 247; 248; 27/28 | | | | | |
| 125; 764464761; | 1568 | 3140 | 3965 | n/a | n/a |
| NZ_JYBE01000113.1; | | | | | |
| 249; 250; 27/28 | | | | | |
| 126; 664184565; | 1882 | 3146 | 3965 | n/a | n/a |
| NZ_JOGA01000019.1; | | | | | |
| 251; 252; 27/28 | | | | | |
| 127; 764464761; | 1890 | 3156 | 3965 | n/a | n/a |
| NZ_JYBE01000113.1; | | | | | |
| 253; 254; 27/28 | | | | | |
| 128; 764464761; | 1452 | 2516 | 3867 | n/a | n/a |
| NZ_JYBE01000113.1; | | | | | |
| 255; 256; 27/28 | | | | | |
| 129; 764464761; | 1890 | 3411 | 3965 | n/a | n/a |
| NZ_JYBE01000113.1; | | | | | |
| 257; 258; 27/28 | | | | | |
| 130; 664051798; | 1873 | 3145 | 4269 | n/a | n/a |
| NZ_JNZK01000024.1; | | | | | |
| 259; 260; 27/28 | | | | | |
| 131; 664095100; | 1859 | 3154 | 4248 | n/a | n/a |
| NZ_JOED01000028.1; | | | | | |
| 261; 262; 24/25 | | | | | |
| 132; 664095100; | 1859 | 3147 | 4248 | n/a | n/a |
| NZ_JOED01000028.1; | | | | | |
| 263; 264; 24/25 | | | | | |
| 133; 664095100; | 1852 | 3531 | 4292 | n/a | n/a |
| NZ_JOED01000028.1; | | | | | |
| 265; 266; 24/25 | | | | | |
| 134; 664095100; | 1852 | 3123 | 4248 | n/a | n/a |
| NZ_JOED01000028.1; | | | | | |
| 267; 268; 24/25 | | | | | |
| 135; 664095100; | 1852 | 3649 | 4248 | n/a | n/a |
| NZ_JOED01000028.1; | | | | | |
| 269; 270; 24/25 | | | | | |
| 136; 664095100; | 1852 | 3144 | 4248 | n/a | n/a |
| NZ_JOED01000028.1; | | | | | |
| 271; 272; 24/25 | | | | | |
| 137; 664095100; | 1852 | 3141 | 4248 | n/a | n/a |
| NZ_JOED01000028.1; | | | | | |
| 273; 274; 24/25 | | | | | |
| 138; 664095100; | 1852 | 3534 | 4248 | n/a | n/a |
| NZ_JOED01000028.1; | | | | | |
| 275; 276; 24/25 | | | | | |
| 139; 664095100; | 1859 | 3530 | 4248 | n/a | n/a |
| NZ_JOED01000028.1; | | | | | |
| 277; 278; 24/25 | | | | | |
| 140; 664095100; | 1883 | 3527 | 4276 | n/a | n/a |
| NZ_JOED01000028.1; | | | | | |
| 279; 280; 24/25 | | | | | |
| 141; 664095100; | 1852 | 3391 | 4248 | n/a | n/a |
| NZ_JOED01000028.1; | | | | | |
| 281; 282; 24/25 | | | | | |
| 142; 664095100; | 1852 | 3528 | 4248 | n/a | n/a |
| NZ_JOED01000028.1; | | | | | |
| 283; 284; 24/25 | | | | | |
| 143; 484070161; | 1708 | 2862 | 4109 | n/a | n/a |
| NZ_KB898999.1; 285; | | | | | |
| 286; 24/25 | | | | | |
| 144; 664095100; | 1852 | 3529 | 4248 | n/a | n/a |
| NZ_JOED01000028.1; | | | | | |
| 287; 288; 24/25 | | | | | |
| 145; 664095100; | 1883 | 3651 | 4276 | n/a | n/a |
| NZ_JOED01000028.1; | | | | | |
| 289; 290; 24/25 | | | | | |
| 146; 664095100; | 1878 | 3152 | 4247 | n/a | n/a |
| NZ_JOED01000028.1; | | | | | |
| 291; 292; 24/25 | | | | | |
| 147; 664095100; | 1851 | 3153 | 4247 | n/a | n/a |
| NZ_JOED01000028.1; | | | | | |
| 293; 294; 24/25 | | | | | |
| 148; 664049400; | 1872 | 3176 | 4268 | n/a | n/a |
| NZ_JOEZ01000021.1; | | | | | |
| 295; 296; 24/25 | | | | | |
| 149; 695845602; | 1343 | 2375 | 3782 | n/a | n/a |
| NZ_JNWU01000018.1; | | | | | |
| 297; 298; 24/25 | | | | | |
| 150; 695845602; | 1645 | 3404 | 4413 | n/a | n/a |
| NZ_JNWU01000018.1; | | | | | |
| 299; 300; 24/25 | | | | | |
| 151; 695845602; | 1916 | 3143 | 4304 | n/a | n/a |
| NZ_JNWU01000018.1; | | | | | |
| 301; 302; 24/25 | | | | | |
| 152; 943927948; | 1902 | 3150 | 4296 | n/a | n/a |
| NZ_LIQV01000315.1; | | | | | |
| 303; 304; 24/25 | | | | | |
| 153; 654969845; | 2256 | 3647 | 4119 | n/a | n/a |
| NZ_ARPF01000020.1; | | | | | |
| 305; 306; 16/17 | | | | | |
| 154; 664095100; | 1869 | 3149 | 4265 | n/a | n/a |
| NZ_JOED01000028.1; | | | | | |
| 307; 308; 24/25 | | | | | |
| 155; 664021017; | 1869 | 3149 | 4265 | n/a | n/a |
| NZ_JOEM01000009.1; | | | | | |
| 309; 310; 26/27 | | | | | |
| 156; 664095100; | 1702 | 2856 | 4108 | n/a | n/a |
| NZ_JOED01000028.1; | | | | | |
| 311; 312; 24/25 | | | | | |
| 157; 654969845; | 1701 | 2855 | 4107 | n/a | n/a |
| NZ_ARPF01000020.1; | | | | | |
| 313; 314; 16/17 | | | | | |
| 158; 654969845; | 1821 | 3142 | 4119 | n/a | n/a |
| NZ_ARPF01000020.1; | | | | | |
| 315; 316; 16/17 | | | | | |
| 159; 221717172; | 1391 | 2441 | 3829 | n/a | n/a |
| DS999644.1; 317; 318; | | | | | |
| 27/28 | | | | | |
| 160; 315497051; | 1334 | 2360 | n/a | n/a | n/a |
| NC_014816.1; 319; 320; | | | | | |
| 28/29 | | | | | |
| 161; 315497051; | 1612 | 3364 | n/a | n/a | n/a |
| NC_014816.1; 321; 322; | | | | | |
| 28/29 | | | | | |
| 162; 380356103; | 1368 | 2406 | 3803 | n/a | n/a |
| AB593691.1; 323; 324; | | | | | |
| 26/27 | | | | | |
| 163; 383755859; | 1369 | 2407 | n/a | n/a | n/a |
| NC_017075.1; 325; 326; | | | | | |
| 20/21 | | | | | |
| 164; 383755859; | 1630 | 3401 | n/a | n/a | n/a |
| NC_017075.1; 327; 328; | | | | | |
| 20/21 | | | | | |
| 165; 381171950; | 2146 | 2596 | n/a | n/a | n/a |
| NZ_CAHO01000029.1; | | | | | |
| 329; 330; 29/30 | | | | | |
| 166; 325923334; | 1534 | 2622 | n/a | n/a | n/a |
| NZ_AEQX01000392.1; | | | | | |
| 331; 332; 26/27 | | | | | |
| 167; 325923334; | 1534 | 2622 | n/a | n/a | n/a |
| NZ_AEQX01000392.1; | | | | | |
| 333; 334; 28/29 | | | | | |
| 168; 565808720; | 2065 | 2946 | n/a | n/a | n/a |
| NZ_CM002307.1; 335; | | | | | |
| 336; 26/27 | | | | | |
| 169; 565808720; | 2065 | 2946 | n/a | n/a | n/a |
| NZ_CM002307.1; 337; | | | | | |
| 338; 28/29 | | | | | |
| 170; 825139250; | 2099 | 3467 | n/a | n/a | n/a |
| NZ_JZEH01000001.1; | | | | | |
| 339; 340; 26/27 | | | | | |
| 171; 325923334; | 2099 | 3467 | n/a | n/a | n/a |
| NZ_AEQX01000392.1; | | | | | |
| 341; 342; 28/29 | | | | | |
| 172; 507418017; | 2008 | 3314 | n/a | n/a | n/a |
| NZ_APMC02000050.1; | | | | | |
| 343; 344; 26/27 | | | | | |
| 173; 746486416; | 2008 | 3314 | n/a | n/a | n/a |
| NZ_KL638873.1; 345; | | | | | |
| 346; 28/29 | | | | | |
| 174; 746366822; | 2010 | 3316 | n/a | n/a | n/a |
| NZ_JSZF01000067.1; | | | | | |
| 347; 348; 26/27 | | | | | |
| 175; 746366822; | 2010 | 3316 | n/a | n/a | n/a |
| NZ_JSZF01000067.1; | | | | | |
| 349; 350; 28/29 | | | | | |
| 176; 825156557; | 2100 | 3468 | n/a | n/a | n/a |
| NZ_JZEI01000001.1; | | | | | |
| 351; 352; 25/26 | | | | | |
| 177; 920684790; | 2100 | 3468 | n/a | n/a | n/a |
| NZ_LHBW01000046.1; | | | | | |
| 353; 354; 28/29 | | | | | |
| 178; 507418017; | 2091 | 3451 | n/a | n/a | n/a |
| NZ_APMC02000050.1; | | | | | |
| 355; 356; 26/27 | | | | | |
| 179; 810489403; | 2091 | 3451 | n/a | n/a | n/a |
| NZ_CP011256.1; 357; | | | | | |
| 358; 28/29 | | | | | |
| 180; 746366822; | 2006 | 3312 | n/a | n/a | n/a |
| NZ_JSZF01000067.1; | | | | | |
| 359; 360;26/27 | | | | | |
| 181; 746366822; | 2006 | 3312 | n/a | n/a | n/a |
| NZ_JSZF01000067.1; | | | | | |
| 361; 362; 28/29 | | | | | |
| 182; 507418017; | 2007 | 3313 | n/a | n/a | n/a |
| NZ_APMC02000050.1; | | | | | |
| 363; 364; 26/27 | | | | | |
| 183; 507418017; | 2007 | 3313 | n/a | n/a | n/a |
| NZ_APMC02000050.1; | | | | | |
| 365; 366; 28/29 | | | | | |
| 184; 507418017; | 1665 | 3323 | n/a | n/a | n/a |
| NZ_APMC02000050.1; | | | | | |
| 367; 368; 26/27 | | | | | |
| 185; 507418017; | 1665 | 3323 | n/a | n/a | n/a |
| NZ_APMC02000050.1; | | | | | |
| 369; 370; 28/29 | | | | | |
| 186; 507418017; | 2007 | 3386 | n/a | n/a | n/a |
| NZ_APMC02000050.1; | | | | | |
| 371; 372; 26/27 | | | | | |
| 187; 507418017; | 2007 | 3386 | n/a | n/a | n/a |
| NZ_APMC02000050.1; | | | | | |
| 373; 374; 28/29 | | | | | |
| 188; 746494072; | 2009 | 3315 | n/a | n/a | n/a |
| NZ_KL638866.1; 375; | | | | | |
| 376; 26/27 | | | | | |
| 189; 507418017; | 2009 | 3315 | n/a | n/a | n/a |
| NZ_APMC02000050.1; | | | | | |
| 377; 378; 28/29 | | | | | |
| 190; 507418017; | 1665 | 2804 | n/a | n/a | n/a |
| NZ_APMC02000050.1; | | | | | |
| 379; 380; 26/27 | | | | | |
| 191; 507418017; | 1665 | 2804 | n/a | n/a | n/a |
| NZ_APMC02000050.1; | | | | | |
| 381; 382; 28/29 | | | | | |
| 192; 507418017; | 2245 | 3633 | n/a | n/a | n/a |
| NZ_APMC02000050.1; | | | | | |
| 383; 384; 26/27 | | | | | |
| 193; 920684790; | 2245 | 3633 | n/a | n/a | n/a |
| NZ_LHBW01000046.1; | | | | | |
| 385; 386; 28/29 | | | | | |
| 194; 941965142; | 1477 | 2551 | n/a | n/a | n/a |
| NZ_LKIT01000002.1; | | | | | |
| 387; 388; 26/27 | | | | | |
| 195; 941965142; | 1477 | 2551 | n/a | n/a | n/a |
| NZ_LKIT01000002.1; | | | | | |
| 389; 390; 29/30 | | | | | |
| 196; 893711378; | 1574 | 2663 | n/a | n/a | n/a |
| NZ_KQ236029.1; 391; | | | | | |
| 392; 23/24 | | | | | |
| 197; 893711378; | 2125 | 3501 | n/a | n/a | n/a |
| NZ_KQ236029.1; 393; | | | | | |
| 394; 23/24 | | | | | |
| 198; 893711378; | 1676 | 2818 | n/a | n/a | n/a |
| NZ_KQ236029.1; 395; | | | | | |
| 396; 23/24 | | | | | |
| 199; 763092879; | 2066 | 3403 | n/a | n/a | n/a |
| NZ_JXZE01000003.1; | | | | | |
| 397; 398; 23/24 | | | | | |
| 200; 103485498; | 1320 | 2342 | n/a | n/a | n/a |
| NC_008048.1; 399; 400; | | | | | |
| 18/19 | | | | | |
| 201; 103485498; | 1320 | 2342 | n/a | n/a | n/a |
| NC_008048.1; 401; 402; | | | | | |
| 21/22 | | | | | |
| 202; 103485498; | 2134 | 3357 | n/a | n/a | n/a |
| NC_008048.1; 403; 404; | | | | | |
| 18/19 | | | | | |
| 203; 103485498; | 2134 | 3357 | n/a | n/a | n/a |
| NC_008048.1; 405; 406; | | | | | |
| 21/22 | | | | | |
| 204; 924898949; | 1361 | 2396 | n/a | n/a | n/a |
| NZ_CP009452.1; 407; | | | | | |
| 408; 21/22 | | | | | |
| 205; 738613868; | 1964 | 3217 | n/a | n/a | n/a |
| NZ_JYZ01000002.1; | | | | | |
| 409; 410; 21/22 | | | | | |
| 206; 834156795; | n/a | 2497 | n/a | n/a | n/a |
| BBRO01000001.1; 411; | | | | | |
| 412; 12/13 | | | | | |
| 207; 834156795; | n/a | 2506 | n/a | n/a | n/a |
| BBRO01000001.1; 413; | | | | | |
| 414; 12/13 | | | | | |
| 208; 834156795; | 1985 | 3251 | n/a | n/a | n/a |
| BBRO01000001.1; 415; | | | | | |
| 416; 12/13 | | | | | |
| 209; 924898949; | 2255 | 3646 | n/a | n/a | n/a |
| NZ_CP009452.1; 417; | | | | | |
| 418; 21/22 | | | | | |
| 210; 937372567; | 2281 | 3689 | n/a | n/a | n/a |
| NZ_CP012700.1; 419; | | | | | |
| 420; 20/21 | | | | | |
| 211; 834156795; | 1434 | 2495 | n/a | n/a | n/a |
| BBRO01000001.1; 421; | | | | | |
| 422; 21/22 | | | | | |
| 212; 834156795; | 1434 | 2495 | n/a | n/a | n/a |
| BBRO01000001.1; 423; | | | | | |
| 424; 12/13 | | | | | |
| 213; 103485498; | 1321 | 2343 | n/a | n/a | n/a |
| NC_008048.1; 425; 426; | | | | | |
| 21/22 | | | | | |
| 214; 103485498; | 2028 | 3358 | n/a | n/a | n/a |
| NC_008048.1; 427; 428; | | | | | |
| 21/22 | | | | | |
| 215; 167621728; | 1597 | 2696 | n/a | n/a | n/a |
| NC_010335.1; 429; 430; | | | | | |
| 23/24 | | | | | |
| 216; 167621728; | 1597 | 2696 | n/a | n/a | n/a |
| NC_010335.1; 431; 432; | | | | | |
| 23/24 | | | | | |
| 217; 167621728; | 1597 | 2696 | n/a | n/a | n/a |
| NC_010335.1; 433; 434; | | | | | |
| 23/24 | | | | | |
| 218; 196476886; | 1326 | 2351 | n/a | n/a | n/a |
| CP000747.1; 435; 436; | | | | | |
| 16/17 | | | | | |
| 219; 295429362; | 1331 | 2356 | n/a | n/a | n/a |
| CP002008.1; 437; 438; | | | | | |
| 21/22 | | | | | |
| 220; 295429362; | 1331 | 2356 | n/a | n/a | n/a |
| CP002008.1; 439; 440; | | | | | |
| 18/19 | | | | | |
| 221; 295429362; | 1331 | 2356 | n/a | n/a | n/a |
| CP002008.1; 441; 442; | | | | | |
| 23/24 | | | | | |
| 222; 654573246; | 1817 | 3554 | n/a | n/a | n/a |
| NZ_AUEO01000025.1; | | | | | |
| 443; 444; 21/22 | | | | | |
| 223; 654573246; | 1817 | 3554 | n/a | n/a | n/a |
| NZ_AUEO01000025.1; | | | | | |
| 445; 446; 18/19 | | | | | |
| 224; 654573246; | 1817 | 3554 | n/a | n/a | n/a |
| NZ_AUEO01000025.1; | | | | | |
| 447; 448; 41/42 | | | | | |
| 225; 297196766; | 1389 | 2437 | 3825 | n/a | n/a |
| NZ_CM000951.1; 449; | | | | | |
| 450; 24/25 | | | | | |
| 226; 297196766; | n/a | 3543 | 3944 | n/a | n/a |
| NZ_CM000951.1; 451; | | | | | |
| 452; 24/25 | | | | | |
| 227; 754819815; | 1378 | 2424 | 3817 | n/a | n/a |
| NZ_CDME01000002.1; | | | | | |
| 453; 454; 24/25 | | | | | |
| 228; 754819815; | 1378 | 2424 | 3817 | n/a | n/a |
| NZ_CDME01000002.1; | | | | | |
| 455; 456; 24/25 | | | | | |
| 229; 754819815; | 2042 | 3615 | 4396 | n/a | n/a |
| NZ_CDME01000002.1; | | | | | |
| 457; 458; 24/25 | | | | | |
| 230; 754819815; | 2042 | 3615 | 4396 | n/a | n/a |
| NZ_CDME01000002.1; | | | | | |
| 459; 460; 24/25 | | | | | |
| 231; 487385965; | 1719 | 2878 | 4123 | n/a | n/a |
| NZ_KB911613.1; 461; | | | | | |
| 462; 23/24 | | | | | |
| 232; 487385965; | 1719 | 2878 | 4123 | n/a | n/a |
| NZ_KB911613.1; 463; | | | | | |
| 464; 22/23 | | | | | |
| 233; 458977979; | 1403 | 2457 | 3837 | n/a | n/a |
| NZ_AORZ01000024.1; | | | | | |
| 465; 466; 16/17 | | | | | |
| 234; 458977979; | 1528 | 3549 | 3930 | n/a | n/a |
| NZ_AORZ01000024.1; | | | | | |
| 467; 468; 16/17 | | | | | |
| 235; 825314728; | 2239 | 3470 | n/a | n/a | n/a |
| NZ_LASZ01000003.1; | | | | | |
| 469; 470; 26/27 | | | | | |
| 236; 483972948; | 1704 | 2858 | 4185 | n/a | n/a |
| NZ_KB891808.1; 471; | | | | | |
| 472; 28/29 | | | | | |
| 237; 937505789; | 1476 | 2550 | n/a | n/a | n/a |
| NZ_LJGM01000026.1; | | | | | |
| 473; 474; 26/27 | | | | | |
| 238; 938883590; | 2283 | 3692 | n/a | n/a | n/a |
| NZ_CP012900.1; 475; | | | | | |
| 476; 25/26 | | | | | |
| 239; 663737675; | 2191 | 3572 | 4263 | n/a | n/a |
| NZ_JOJF01000002.1; | | | | | |
| 477; 478; 29/30 | | | | | |
| 240; 835885587; | 2104 | 3593 | n/a | n/a | n/a |
| NZ_KN265462.1; 479; | | | | | |
| 480; 26/27 | | | | | |
| 241; 825314716; | 2101 | 3469 | n/a | n/a | n/a |
| NZ_LASZ01000002.1; | | | | | |
| 481; 482; 26/27 | | | | | |
| 242; 67639376; | 1449 | 2512 | n/a | n/a | n/a |
| NZ_AAHO01000116.1; | | | | | |
| 483; 484; 28/29 | | | | | |
| 243; 835885587; | 1448 | 2510 | n/a | n/a | n/a |
| NZ_KN265462.1; 485; | | | | | |
| 486; 33/34 | | | | | |
| 244; 433601838; | n/a | 2758 | 4044 | n/a | n/a |
| NC_019673.1; 487; 488; | | | | | |
| 26/27 | | | | | |
| 245; 653330442; | 1812 | 3032 | n/a | n/a | n/a |
| NZ_KE386531.1; 489; | | | | | |
| 490; 26/27 | | | | | |
| 246; 389798210; | 1543 | 2633 | n/a | n/a | n/a |
| NZ_AJXV01000032.1; | | | | | |
| 491; 492; 26/27 | | | | | |
| 247; 469816339; | 1643 | 2769 | n/a | n/a | n/a |
| NC_020541.1; 493; 494; | | | | | |
| 26/27 | | | | | |
| 248; 653308965; | 1809 | 3029 | n/a | n/a | n/a |
| NZ_AXBJ01000026.1; | | | | | |
| 495; 496; 24/25 | | | | | |
| 249; 919546651; | n/a | 3629 | n/a | n/a | n/a |
| NZ_JOEL01000060.1; | | | | | |
| 497; 498; 27/28 | | | | | |
| 250; 653321547; | 1810 | 3030 | n/a | n/a | n/a |
| NZ_ATYF01000013.1; | | | | | |
| 499; 500; 26/27 | | | | | |
| 251; 332527785; | 1564 | 2658 | n/a | n/a | n/a |
| NZ_AEWG01000155.1; | | | | | |
| 501; 502; 20/21 | | | | | |
| 252; 269954810; | 1605 | 3541 | 4000 | n/a | n/a |
| NC_013530.1; 503; 504; | | | | | |
| 20/21 | | | | | |
| 253; 943674269; | 1656 | 3565 | 4070 | n/a | n/a |
| NZ_LIQO01000205.1; | | | | | |
| 505; 506; 21/22 | | | | | |
| 254; 663414324; | 1656 | 2794 | 4070 | n/a | n/a |
| NZ_JOHQ01000068.1; | | | | | |
| 507; 508; 21/22 | | | | | |
| 255; 943674269; | 1656 | 3568 | 4070 | n/a | n/a |
| NZ_LIQO01000205.1; | | | | | |
| 509; 510; 21/22 | | | | | |
| 256; 269954810; | 1328 | 2353 | 3765 | n/a | n/a |
| NC_013530.1; 511; 512; | | | | | |
| 20/21 | | | | | |
| 257; 937505789; | 1760 | 3516 | n/a | n/a | n/a |
| NZ_LJGM01000026.1; | | | | | |
| 513; 514; 26/27 | | | | | |
| 258; 663414324; | 1864 | 3563 | 4070 | n/a | n/a |
| NZ_JOHQ01000068.1; | | | | | |
| 515; 516; 21/22 | | | | | |
| 259; 663414324; | 1656 | 3575 | 4070 | n/a | n/a |
| NZ_JOHQ01000068.1; | | | | | |
| 517; 518; 21/22 | | | | | |
| 260; 389759651; | 1548 | 3229 | n/a | n/a | n/a |
| NZ_AJXS01000437.1; | | | | | |
| 519; 520; 26/27 | | | | | |
| 261; 928998800; | 2274 | 3675 | n/a | n/a | n/a |
| NZ_BBYR01000083.1; | | | | | |
| 521; 522; 16/17 | | | | | |
| 262; 943674269; | 1656 | 3673 | 4070 | n/a | n/a |
| NZ_LIQO01000205.1; | | | | | |
| 523; 524; 21/22 | | | | | |
| 263; 856992287; | 2113 | 3484 | 4458 | n/a | n/a |
| NZ_LFKW01000127.1; | | | | | |
| 525; 526; 20/21 | | | | | |
| 264; 938956730; | 2285 | 3694 | n/a | n/a | n/a |
| NZ_CP009429.1; 527; | | | | | |
| 528; 19/20 | | | | | |
| 265; 563282524; | 1419 | 2474 | n/a | n/a | n/a |
| AYSC01000019.1; 529; | | | | | |
| 530; 22/23 | | | | | |
| 266; 399058618; | 1545 | 2636 | n/a | n/a | n/a |
| NZ_AKKE01000021.1; | | | | | |
| 531; 532; 22/23 | | | | | |
| 267; 937372567; | n/a | 3690 | n/a | n/a | n/a |
| NZ_CP012700.1; 533; | | | | | |
| 534; 19/20 | | | | | |
| 268; 825353621; | 2102 | 3471 | 4445 | n/a | n/a |
| NZ_LAYX01000011.1; | | | | | |
| 535; 536; 21/22 | | | | | |
| 269; 937505789; | 2282 | 3691 | n/a | n/a | n/a |
| NZ_LJGM01000026.1; | | | | | |
| 537; 538; 26/27 | | | | | |
| 270; 739702045; | 1446 | 2508 | n/a | n/a | n/a |
| NZ_JNFC01000030.1; | | | | | |
| 539; 540; 18/19 | | | | | |
| 271; 484867900; | n/a | 3448 | 4110 | n/a | n/a |
| NZ_AGNH01000612.1; | | | | | |
| 541; 542; 15/16 | | | | | |
| 272; 162960844; | 1989 | 3257 | 4349 | n/a | n/a |
| NC_003155.4; 543; 544; | | | | | |
| 23/24 | | | | | |
| 273; 162960844; | n/a | 2403 | 3800 | n/a | n/a |
| NC_003155.4; 545; 546; | | | | | |
| 23/24 | | | | | |
| 274; 399069941; | 1544 | 2635 | n/a | n/a | n/a |
| NZ_AKKF01000033.1; | | | | | |
| 547; 548; 22/23 | | | | | |
| 275; 399069941; | 1544 | 2635 | n/a | n/a | n/a |
| NZ_AKKF01000033.1; | | | | | |
| 549; 550; 22/23 | | | | | |
| 276; 738615271; | 1428 | 2485 | n/a | n/a | n/a |
| NZ_JFYZ01000008.1; | | | | | |
| 551; 552; 22/23 | | | | | |
| 277; 739659070; | 1445 | 2507 | n/a | n/a | n/a |
| NZ_JNFD01000017.1; | | | | | |
| 553; 554; 19/20 | | | | | |
| 278; 749188513; | 2011 | 3317 | n/a | n/a | n/a |
| NZ_CP009122.1; 555; | | | | | |
| 556; 19/20 | | | | | |
| 279; 345007964; | 1624 | 3548 | 4025 | n/a | n/a |
| NC_015957.1; 557; 558; | | | | | |
| 24/25 | | | | | |
| 280; 345007964; | 1624 | 3548 | 4025 | n/a | n/a |
| NC_015957.1; 559; 560; | | | | | |
| 24/25 | | | | | |
| 281; 345007964; | 1337 | 2364 | 3771 | n/a | n/a |
| NC_015957.1; 561; 562; | | | | | |
| 24/25 | | | | | |
| 282; 345007964; | 1337 | 2364 | 3771 | n/a | n/a |
| NC_015957.1; 563; 564; | | | | | |
| 24/25 | | | | | |
| 283; 928998724; | 1436 | 2498 | n/a | n/a | n/a |
| NZ_BBYR01000007.1; | | | | | |
| 565; 566; 19/20 | | | | | |
| 284; 484007841; | n/a | 2822 | 4087 | n/a | n/a |
| NZ_ANAD01000138.1; | | | | | |
| 567; 568; 20/21 | | | | | |
| 285; 162960844; | 1583 | 3256 | 4348 | n/a | n/a |
| NC_003155.4; 569; 570; | | | | | |
| 21/22 | | | | | |
| 286; 162960844; | 1366 | 2404 | 3801 | n/a | n/a |
| NC_003155.4; 571; 572; | | | | | |
| 21/22 | | | | | |
| 287; 662133033; | 1894 | 3271 | 4287 | n/a | n/a |
| NZ_KL570321.1; 573; | | | | | |
| 574; 21/22 | | | | | |
| 288; 662133033; | 1850 | 3494 | 4246 | n/a | n/a |
| NZ_KL570321.1; 575; | | | | | |
| 576; 21/22 | | | | | |
| 289; 487404592; | 1725 | 2886 | 4131 | n/a | n/a |
| NZ_ARVW01000001.1; | | | | | |
| 577; 578; 22/23 | | | | | |
| 290; 739659070; | 2215 | 3245 | n/a | n/a | n/a |
| NZ_JNFD01000017.1; | | | | | |
| 579; 580; 19/20 | | | | | |
| 291; 702808005; | 1925 | 3167 | 4311 | n/a | n/a |
| NZ_JNZA01000041.1; | | | | | |
| 581; 582; 21/22 | | | | | |
| 292; 664277815; | 1889 | 3574 | 4281 | n/a | n/a |
| NZ_JOIX01000041.1; | | | | | |
| 583; 584; 21/22 | | | | | |
| 293; 499136900; | 1972 | 3234 | 4345 | n/a | n/a |
| NZ_ASJB01000015.1; | | | | | |
| 585; 586; 20/21 | | | | | |
| 294; 487404592; | 1725 | 2886 | 4131 | n/a | n/a |
| NZ_ARVW01000001.1; | | | | | |
| 587; 588; 22/23 | | | | | |
| 295; 716912366; | 1928 | 3172 | 4314 | n/a | n/a |
| NZ_JRHJ01000016.1; | | | | | |
| 589; 590; 21/22 | | | | | |
| 296; 381200190; | 1567 | 2660 | 3964 | n/a | n/a |
| NZ_JH164855.1; 591; | | | | | |
| 592; 19/20 | | | | | |
| 297; 663300513; | 1856 | 3255 | 4252 | n/a | n/a |
| NZ_JNZY01000033.1; | | | | | |
| 593; 594; 21/22 | | | | | |
| 298; 822214995; | 1355 | 2388 | 3792 | n/a | n/a |
| NZ_CP007699.1; 595; | | | | | |
| 596; 21/22 | | | | | |
| 299; 664013282; | 1868 | 3261 | 4264 | n/a | n/a |
| NZ_JOAP01000011.1; | | | | | |
| 597; 598; 12/13 | | | | | |
| 300; 822214995; | 2095 | 3460 | 4441 | n/a | n/a |
| NZ_CP007699.1; 599; | | | | | |
| 600; 21/22 | | | | | |
| 301; 514916021; | 1409 | 2463 | 3841 | n/a | n/a |
| NZ_AOPZ01000017.1; | | | | | |
| 601; 602; 21/22 | | | | | |
| 302; 514916021; | 1658 | 3258 | 4071 | n/a | n/a |
| NZ_AOPZ01000017.1; | | | | | |
| 603; 604; 21/22 | | | | | |
| 303; 663421576; | 1865 | 3579 | 4260 | n/a | n/a |
| NZ_JOGE01000134.1; | | | | | |
| 605; 606; 21/22 | | | | | |
| 304; 928897596; | 2272 | 3672 | 4538 | n/a | n/a |
| NZ_LGKG01000207.1; | | | | | |
| 607; 608; 21/22 | | | | | |
| 305; 484007121; | n/a | 2756 | 4042 | n/a | n/a |
| NZ_ANAC01000010.1; | | | | | |
| 609; 610; 29/30 | | | | | |
| 306; 484007121; | 1779 | 3377 | 4042 | n/a | n/a |
| NZ_ANAC01000010.1; | | | | | |
| 611; 612; 29/30 | | | | | |
| 307; 646523831; | 2241 | 2972 | n/a | n/a | n/a |
| NZ_BATN01000047.1; | | | | | |
| 613; 614; 18/19 | | | | | |
| 308; 484007121; | 1779 | 2820 | 4042 | n/a | n/a |
| NZ_ANAC01000010.1; | | | | | |
| 615; 616; 29/30 | | | | | |
| 309; 651281457; | 1782 | 3556 | 4488 | n/a | n/a |
| NZ_JADG01000010.1; | | | | | |
| 617; 618; 19/20 | | | | | |
| 310; 664428976; | 1854 | 3080 | 4250 | n/a | n/a |
| NZ_KL585179.1; 619; | | | | | |
| 620; 21/22 | | | | | |
| 311; 926412104; | 2266 | 3663 | 4533 | n/a | n/a |
| NZ_LGDY01000113.1; | | | | | |
| 621; 622; 18/19 | | | | | |
| 312; 703210604; | n/a | 3169 | n/a | n/a | n/a |
| NZ_JNYM01000124.1; | | | | | |
| 623; 624; 44/45 | | | | | |
| 313; 471319476; | 1647 | 2774 | 4059 | n/a | n/a |
| NC_020504.1; 625; 626; | | | | | |
| 21/22 | | | | | |
| 314; 485454803; | 2057 | 3525 | 4408 | n/a | n/a |
| NZ_AFRP01001656.1; | | | | | |
| 627; 628; 21/22 | | | | | |
| 315; 664487325; | 1896 | 3157 | 4290 | n/a | n/a |
| NZ_JOJI01000036.1; | | | | | |
| 629; 630; 29/30 | | | | | |
| 316; 297189896; | 1390 | 2438 | 3826 | n/a | n/a |
| NZ_CM000950.1; 631; | | | | | |
| 632; 21/22 | | | | | |
| 317; 297189896; | 1531 | 3268 | 3933 | n/a | n/a |
| NZ_CM000950.1; 633; | | | | | |
| 634; 21/22 | | | | | |
| 318; 398790069; | 2040 | 3371 | 4394 | n/a | n/a |
| NZ_JH725387.1; 635; | | | | | |
| 636; 21/22 | | | | | |
| 319; 754221033; | n/a | 3277 | 4362 | n/a | n/a |
| NZ_CP007574.1; 637; | | | | | |
| 638; 22/23 | | | | | |
| 320; 928998724; | 2273 | 3674 | n/a | n/a | n/a |
| NZ_BBYR01000007.1; | | | | | |
| 639; 640; 19/20 | | | | | |
| 321; 931609467; | n/a | 3683 | 4543 | n/a | n/a |
| NZ_CP012752.1; 641; | | | | | |
| 642; 24/25 | | | | | |
| 322; 484017897; | 1776 | 2829 | 4124 | n/a | n/a |
| NZ_ANBB01000025.1; | | | | | |
| 643; 644; 20/21 | | | | | |
| 323; 943388237; | 2055 | 3606 | 4406 | n/a | n/a |
| NZ_LIQD01000001.1; | | | | | |
| 645; 646; 21/22 | | | | | |
| 324; 398790069; | 1536 | 2625 | 3938 | n/a | n/a |
| NZ_JH725387.1; 647; | | | | | |
| 648; 21/22 | | | | | |
| 325; 224581107; | 1517 | 2602 | 3926 | n/a | n/a |
| NZ_GG657757.1; 649; | | | | | |
| 650; 19/20 | | | | | |
| 326; 664245663; | 1888 | 3109 | 4279 | n/a | n/a |
| NZ_JODF01000003.1; | | | | | |
| 651; 652; 21/22 | | | | | |
| 327; 664026629; | 1870 | 3096 | 4266 | n/a | n/a |
| NZ_JOAP01000049.1; | | | | | |
| 653; 654; 21/22 | | | | | |
| 328; 764439507; | 1848 | 3410 | 4245 | n/a | n/a |
| NZ_JRKI01000027.1; | | | | | |
| 655; 656; 21/22 | | | | | |
| 329; 662059070; | 1845 | 3076 | 4242 | n/a | n/a |
| NZ_KL571162.1; 657; | | | | | |
| 658; 29/30 | | | | | |
| 330; 739830264; | 1991 | 3260 | 4352 | n/a | n/a |
| NZ_JOJE01000040.1; | | | | | |
| 659; 660; 21/22 | | | | | |
| 331; 662063073; | 2082 | 3432 | 4426 | n/a | n/a |
| NZ_JNXV01000303.1; | | | | | |
| 661; 662; 22/23 | | | | | |
| 332; 664141810; | 1881 | 3105 | 4275 | n/a | n/a |
| NZ_JOCQ01000106.1; | | | | | |
| 663; 664; 29/30 | | | | | |
| 333; 799161588; | n/a | 2525 | 3873 | n/a | n/a |
| NZ_JZWZ01000076.1; | | | | | |
| 665; 666; 25/26 | | | | | |
| 334; 664523889; | 1897 | 3603 | 4291 | n/a | n/a |
| NZ_JOFH01000020.1; | | | | | |
| 667; 668; 23/24 | | | | | |
| 335; 754862786; | 1767 | 2968 | 4177 | n/a | n/a |
| NZ_CP007155.1; 669; | | | | | |
| 670; 40/41 | | | | | |
| 336; 655416831; | 1828 | 3054 | 4226 | n/a | n/a |
| NZ_KE386846.1; 671; | | | | | |
| 672; 20/21 | | | | | |
| 337; 662063073; | n/a | 3077 | 4243 | n/a | n/a |
| NZ_JNXV01000303.1; | | | | | |
| 673; 674; 22/23 | | | | | |
| 338; 664523889; | 1993 | 3552 | 4354 | n/a | n/a |
| NZ_JOFH01000020.1; | | | | | |
| 675; 676; 23/24 | | | | | |
| 339; 663122276; | 1853 | 3252 | 4249 | n/a | n/a |
| NZ_JOFJ01000001.1; | | | | | |
| 677; 678; 20/21 | | | | | |
| 340; 654239557; | 1814 | 3269 | 4213 | n/a | n/a |
| NZ_AZWL01000018.1; | | | | | |
| 679; 680; 21/22 | | | | | |
| 341; 926344107; | 2260 | 3654 | 4525 | n/a | n/a |
| NZ_LGEA01000058.1; | | | | | |
| 681; 682; 19/20 | | | | | |
| 342; 765016627; | 2074 | 3416 | 4416 | n/a | n/a |
| NZ_LK022849.1; 683; | | | | | |
| 684; 22/23 | | | | | |
| 343; 765016627; | 2074 | 3416 | 4416 | n/a | n/a |
| NZ_LK022849.1; 685; | | | | | |
| 686; 22/23 | | | | | |
| 344; 755908329; | 1353 | 2385 | 3790 | n/a | n/a |
| CP007219.1; 687; 688; | | | | | |
| 20/21 | | | | | |
| 345; 664061406; | 1863 | 3668 | 3923 | n/a | n/a |
| NZ_JOES01000059.1; | | | | | |
| 689; 690; 29/30 | | | | | |
| 346; 799161588; | n/a | 3620 | 4431 | n/a | n/a |
| NZ_JZWZ01000076.1; | | | | | |
| 691; 692; 25/26 | | | | | |
| 347; 664061406; | 1514 | 3103 | 3923 | n/a | n/a |
| NZ_JOES01000059.1; | | | | | |
| 693; 694; 29/30 | | | | | |
| 348; 664434000; | 1516 | 2601 | 3925 | n/a | n/a |
| NZ_JOIA01001078.1; | | | | | |
| 695; 696; 21/22 | | | | | |
| 349; 429195484; | 2120 | 2653 | 3959 | n/a | n/a |
| NZ_AEJC01000118.1; | | | | | |
| 697; 698; 22/23 | | | | | |
| 350; 664325162; | 1892 | 3112 | 4284 | n/a | n/a |
| NZ_JOJB01000032.1; | | | | | |
| 699; 700; 21/22 | | | | | |
| 351; 664061406; | 1875 | 3160 | 3923 | n/a | n/a |
| NZ_JOES01000059.1; | | | | | |
| 701; 702; 29/30 | | | | | |
| 352; 657301257; | 2070 | 3412 | 4236 | n/a | n/a |
| NZ_AZSD01000480.1; | | | | | |
| 703; 704; 21/22 | | | | | |
| 353; 657301257; | n/a | 3486 | 4236 | n/a | n/a |
| NZ_AZSD01000480.1; | | | | | |
| 705; 706; 21/22 | | | | | |
| 354; 458984960; | 1529 | 3550 | 3931 | n/a | n/a |
| NZ_AORZ01000079.1; | | | | | |
| 707; 708; 12/13 | | | | | |
| 355; 657301257; | 1835 | 3066 | 4236 | n/a | n/a |
| NZ_AZSD01000480.1; | | | | | |
| 709; 710; 21/22 | | | | | |
| 356; 925315417; | 1863 | 3090 | 3923 | n/a | n/a |
| LGCQ01000244.1; 711; | | | | | |
| 712; 29/30 | | | | | |
| 357; 926371517; | 2262 | 3656 | 4527 | n/a | n/a |
| NZ_LGCW01000271.1; | | | | | |
| 713; 714; 29/30 | | | | | |
| 358; 925315417; | 1514 | 3101 | 3923 | n/a | n/a |
| LGCQ01000244.1; 715; | | | | | |
| 716; 29/30 | | | | | |
| 359; 664325162; | 1858 | 3084 | 4254 | n/a | n/a |
| NZ_JOJB01000032.1; | | | | | |
| 717; 718; 21/22 | | | | | |
| 360; 664061406; | 1514 | 3162 | 3923 | n/a | n/a |
| NZ_JOES01000059.1; | | | | | |
| 719; 720; 29/30 | | | | | |
| 361; 926403453; | 2265 | 3661 | 4530 | n/a | n/a |
| NZ_LGDD01000321.1; | | | | | |
| 721; 722; 21/22 | | | | | |
| 362; 671472153; | 1905 | 2915 | 4152 | n/a | n/a |
| NZ_JOFR01000001.1; | | | | | |
| 723; 724; 21/22 | | | | | |
| 363; 471319476; | 1646 | 2773 | 4058 | n/a | n/a |
| NC_020504.1; 725; 726; | | | | | |
| 18/19 | | | | | |
| 364; 739854483; | 1992 | 3262 | 4353 | n/a | n/a |
| NZ_KL997447.1; 727; | | | | | |
| 728; 21/22 | | | | | |
| 365; 926371520; | n/a | 2540 | 3884 | n/a | n/a |
| NZ_LGCW01000274.1; | | | | | |
| 729; 730; 27/28 | | | | | |
| 366; 485454803; | n/a | 3546 | n/a | n/a | n/a |
| NZ_AFRP01001656.1; | | | | | |
| 731; 732; 21/22 | | | | | |
| 367; 738615271; | 2182 | 3218 | n/a | n/a | n/a |
| NZ_JFYZ01000008.1; | | | | | |
| 733; 734; 21/22 | | | | | |
| 368; 738615271; | 2182 | 3218 | n/a | n/a | n/a |
| NZ_JFYZ01000008.1; | | | | | |
| 735; 736; 21/22 | | | | | |
| 369; 738615271; | 2182 | 3218 | n/a | n/a | n/a |
| NZ_JFYZ01000008.1; | | | | | |
| 737; 738; 22/23 | | | | | |
| 370; 664479796; | n/a | 3120 | n/a | n/a | n/a |
| NZ_JOJI01000005.1; | | | | | |
| 739; 740; 19/20 | | | | | |
| 371; 357397620; | 1628 | 2747 | 4035 | n/a | n/a |
| NC_016111.1; 741; 742; | | | | | |
| 13/14 | | | | | |
| 372; 665604093; | 1904 | 3126 | 4299 | n/a | n/a |
| NZ_JNXR01000023.1; | | | | | |
| 743; 744; 21/22 | | | | | |
| 373; 739674258; | 1981 | 3247 | n/a | n/a | n/a |
| NZ_JQMC01000050.1; | | | | | |
| 745; 746; 23/24 | | | | | |
| 374; 664061406; | 1461 | 2532 | 3876 | n/a | n/a |
| NZ_JOES01000059.1; | | | | | |
| 747; 748; 29/30 | | | | | |
| 375; 664061406; | 1467 | 2538 | 3882 | n/a | n/a |
| NZ_JOES01000059.1; | | | | | |
| 749; 750; 29/30 | | | | | |
| 376; 926371517; | 1469 | 2541 | 3885 | n/a | n/a |
| NZ_LGCW01000271.1; | | | | | |
| 751; 752; 29/30 | | | | | |
| 377; 664244706; | 1886 | 3108 | 4277 | n/a | n/a |
| NZ_JOBD01000002.1; | | | | | |
| 753; 754; 24/25 | | | | | |
| 378; 925315417; | 1463 | 2534 | 3878 | n/a | n/a |
| LGCQ01000244.1; 755; | | | | | |
| 756; 29/30 | | | | | |
| 379; 646529442; | 1769 | 2973 | n/a | n/a | n/a |
| NZ_BATN01000092.1; | | | | | |
| 757; 758; 18/19 | | | | | |
| 380; 906344334; | 2132 | 3513 | n/a | n/a | n/a |
| NZ_LFXA01000002.1; | | | | | |
| 759; 760; 12/13 | | | | | |
| 381; 926344331; | 2261 | 3655 | 4526 | n/a | n/a |
| NZ_LGEA01000105.1; | | | | | |
| 761; 762; 21/22 | | | | | |
| 382; 664421883; | 1893 | 3115 | 4286 | n/a | n/a |
| NZ_JODC01000023.1; | | | | | |
| 763; 764; 21/22 | | | | | |
| 383; 755134941; | 2240 | 3626 | n/a | n/a | n/a |
| NZ_BBPI01000030.1; | | | | | |
| 765; 766; 22/23 | | | | | |
| 384; 663596322; | 1866 | 3602 | 4261 | n/a | n/a |
| NZ_JOEF01000022.1; | | | | | |
| 767; 768; 21/22 | | | | | |
| 385; 664063830; | 1876 | 3098 | 4271 | n/a | n/a |
| NZ_JODT01000002.1; | | | | | |
| 769; 770; 13/14 | | | | | |
| 386; 484203522; | 1691 | 2842 | 4100 | n/a | n/a |
| NZ_AQUI01000002.1; | | | | | |
| 771; 772; 12/13 | | | | | |
| 387; 365867746; | 1394 | 2445 | 3832 | n/a | n/a |
| NZ_AGSW01000272.1; | | | | | |
| 773; 774; 22/23 | | | | | |
| 388; 759802587; | 2059 | 3399 | 4409 | n/a | n/a |
| NZ_CP009438.1; 775; | | | | | |
| 776; 21/22 | | | | | |
| 389; 664325162; | 1358 | 2393 | 3795 | n/a | n/a |
| NZ_JOJB01000032.1; | | | | | |
| 777; 778; 21/22 | | | | | |
| 390; 484008051; | 1680 | 2824 | 4089 | n/a | n/a |
| NZ_ANAD01000197.1; | | | | | |
| 779; 780; 24/25 | | | | | |
| 391; 458848256; | 1540 | 3327 | 3942 | n/a | n/a |
| NZ_AOHO01000055.1; | | | | | |
| 781; 782; 21/22 | | | | | |
| 392; 458848256; | 1402 | 2456 | 3836 | n/a | n/a |
| NZ_AOHO01000055.1; | | | | | |
| 783; 784; 21/22 | | | | | |
| 393; 664478668; | 1855 | 3272 | 4251 | n/a | n/a |
| NZ_JOJI01000002.1; | | | | | |
| 785; 786; 19/20 | | | | | |
| 394; 484008051; | 1778 | 2825 | 4090 | n/a | n/a |
| NZ_ANAD01000197.1; | | | | | |
| 787; 788; 24/25 | | | | | |
| 395; 365867746; | n/a | 3155 | 3946 | n/a | n/a |
| NZ_AGSW01000272.1; | | | | | |
| 789; 790; 22/23 | | | | | |
| 396; 873282818; | n/a | 3487 | 4461 | n/a | n/a |
| NZ_LFEH01000123.1; | | | | | |
| 791; 792; 25/26 | | | | | |
| 397; 664061406; | 1514 | 3382 | 3923 | n/a | n/a |
| NZ_JOES01000059.1; | | | | | |
| 793; 794; 29/30 | | | | | |
| 398; 873282818; | n/a | 3466 | 4234 | n/a | n/a |
| NZ_LFEH01000123.1; | | | | | |
| 795; 796; 25/26 | | | | | |
| 399; 906344339; | 2133 | 3514 | 4471 | n/a | n/a |
| NZ_LFXA01000007.1; | | | | | |
| 797; 798; 19/20 | | | | | |
| 400; 759944049; | 2061 | 3609 | n/a | n/a | n/a |
| NZ_JOAG01000029.1; | | | | | |
| 799; 800; 28/29 | | | | | |
| 401; 557839714; | 1745 | 2913 | n/a | n/a | n/a |
| NZ_AWGF01000010.1; | | | | | |
| 801; 802; 28/29 | | | | | |
| 402; 695870063; | n/a | 3537 | 4306 | n/a | n/a |
| NZ_JNWW01000028.1; | | | | | |
| 803; 804; 23/24 | | | | | |
| 403; 749181963; | 2013 | 3598 | 4368 | n/a | n/a |
| NZ_CP003987.1; 805; | | | | | |
| 806; 12/13 | | | | | |
| 404; 852460626; | 1359 | 2394 | 3796 | n/a | n/a |
| CP011799.1; 807; 808; | | | | | |
| 13/14 | | | | | |
| 405; 374982757; | 1332 | 2357 | 3767 | n/a | 3768 |
| NC_016582.1; 809; 810; | | | | | |
| 13/14 | | | | | |
| 406; 374982757; | 1332 | 2357 | 3767 | n/a | 3768 |
| NC_016582.1; 811; 812; | | | | | |
| 28/29 | | | | | |
| 407; 914607448; | n/a | 2529 | n/a | n/a | n/a |
| NZ_JYNE01000028.1; | | | | | |
| 813; 814; 22/23 | | | | | |
| 408; 663373497; | 1861 | 3088 | 4257 | n/a | n/a |
| NZ_JOFL01000043.1; | | | | | |
| 815; 816; 19/20 | | | | | |
| 409; 764442321; | n/a | 3625 | 4415 | n/a | n/a |
| NZ_JRKI01000041.1; | | | | | |
| 817; 818; 29/30 | | | | | |
| 410; 739702045; | 2214 | 3250 | n/a | n/a | n/a |
| NZ_JNFC01000030.1; | | | | | |
| 819; 820; 18/19 | | | | | |
| 411; 485090585; | n/a | 2870 | 4115 | n/a | n/a |
| NZ_KB907209.1; 821; | | | | | |
| 822; 20/21 | | | | | |
| 412; 764442321; | 1847 | 3586 | 4501 | n/a | n/a |
| NZ_JRKI01000041.1; | | | | | |
| 823; 824; 29/30 | | | | | |
| 413; 514916412; | 1659 | 3591 | 4350 | n/a | n/a |
| NZ_AOPZ01000028.1; | | | | | |
| 825; 826; 33/34 | | | | | |
| 414; 514916412; | 1408 | 2462 | 3840 | n/a | n/a |
| NZ_AOPZ01000028.1; | | | | | |
| 827; 828; 33/34 | | | | | |
| 415; 970574347; | 1839 | 2873 | 4118 | n/a | n/a |
| NZ_LNZF01000001.1; | | | | | |
| 829; 830; 20/21 | | | | | |
| 416; 970574347; | 1768 | 2969 | 4084 | n/a | n/a |
| NZ_LNZI,01000001.1; | | | | | |
| 831; 832; 20/21 | | | | | |
| 417; 906292938; | 1915 | 3139 | n/a | n/a | n/a |
| CXPB01000073.1; 833; | | | | | |
| 834; 18/19 | | | | | |
| 418; 906292938; | 1383 | 2431 | n/a | n/a | n/a |
| CXPB01000073.1; 835; | | | | | |
| 836; 18/19 | | | | | |
| 419; 970574347; | 1662 | 2799 | 4074 | n/a | n/a |
| NZ_LNZF01000001.1; | | | | | |
| 837; 838; 20/21 | | | | | |
| 420; 671525382; | n/a | 3130 | 4496 | n/a | n/a |
| NZ_JODL01000019.1; | | | | | |
| 839; 840; 31/32 | | | | | |
| 421; 652698054; | 1748 | 2934 | 4159 | n/a | n/a |
| NZ_K1912610.1; 841; | | | | | |
| 842; 26/27 | | | | | |
| 422; 652698054; | 1750 | 2936 | 4159 | n/a | n/a |
| NZ_K1912610.1; 843; | | | | | |
| 844; 26/27 | | | | | |
| 423; 756828038; | 2050 | 3381 | 4403 | n/a | n/a |
| NZ_CCNC01000143.1; | | | | | |
| 845; 846; 26/27 | | | | | |
| 424; 662140302; | 2135 | 3356 | 3988 | n/a | n/a |
| NZ_JMUB01000087.1; | | | | | |
| 847; 848; 22/23 | | | | | |
| 425; 751285871; | 2224 | 3342 | 4382 | n/a | n/a |
| NZ_CCNA01000001.1; | | | | | |
| 849; 850; 26/27 | | | | | |
| 426; 662140302; | n/a | 2348 | 3763 | n/a | n/a |
| NZ_JMUB01000087.1; | | | | | |
| 851; 852; 22/23 | | | | | |
| 427; 751292755; | n/a | 3343 | 4381 | n/a | n/a |
| NZ_CCNE01000004.1; | | | | | |
| 853; 854; 26/27 | | | | | |
| 428; 970574347; | n/a | 3419 | 4418 | n/a | n/a |
| NZ_LNZF01000001.1; | | | | | |
| 855; 856; 20/21 | | | | | |
| 429; 484099183; | 1721 | 2880 | 4126 | n/a | n/a |
| NZ_AJTY01001072.1; | | | | | |
| 857; 858; 19/20 | | | | | |
| 430; 484099183; | n/a | 3324 | n/a | n/a | n/a |
| NZ_AJTY01001072.1; | | | | | |
| 859; 860; 19/20 | | | | | |
| 431; 751265275; | n/a | 3340 | 4380 | n/a | n/a |
| NZ_CCMY01000220.1; | | | | | |
| 861; 862; 26/27 | | | | | |
| 432; 662140302; | 2189 | 3079 | 4240 | n/a | n/a |
| NZ_JMUB01000087.1; | | | | | |
| 863; 864; 22/23 | | | | | |
| 433; 428296779; | n/a | 2764 | 4053 | n/a | n/a |
| NC_019751.1; 865; 866; | | | | | |
| 21/22 | | | | | |
| 434; 662140302; | 2162 | 3075 | 4240 | n/a | n/a |
| NZ_JMUB01000087.1; | | | | | |
| 867; 868; 22/23 | | | | | |
| 435; 563312125; | 1319 | 2340 | n/a | n/a | n/a |
| AYTZ01000052.1; 869; | | | | | |
| 870; 31/32 | | | | | |
| 436; 357028583; | n/a | 2621 | 3936 | n/a | n/a |
| NZ_AGSN01000187.1; | | | | | |
| 871; 872; 26/27 | | | | | |
| 437; 655569633; | 1971 | 3057 | 4491 | n/a | n/a |
| NZ_JIA101000002.1; | | | | | |
| 873; 874; 32/33 | | | | | |
| 438; 655569633; | 1971 | 3057 | 4491 | n/a | n/a |
| NZ_JIA101000002.1; | | | | | |
| 875; 876; 43/44 | | | | | |
| 439; 655569633; | 1971 | 3057 | 4491 | n/a | n/a |
| NZ_JI101000002.1; | | | | | |
| 877; 878; 32/33 | | | | | |
| 440; 970574347; | 2017 | 3330 | 4373 | n/a | n/a |
| NZ_LNZF01000001.1; | | | | | |
| 879; 880; 20/21 | | | | | |
| 441; 482849861; | 1563 | 2656 | 3963 | n/a | n/a |
| NZ_AKBU01000001.1; | | | | | |
| 881; 882; 3/4 | | | | | |
| 442; 482849861; | 1506 | 2779 | 3985 | n/a | n/a |
| NZ_AKBU01000001.1; | | | | | |
| 883; 884; 3/4 | | | | | |
| 443; 737350949; | 1945 | 3198 | 4328 | n/a | n/a |
| NZ_APVL01000034.1; | | | | | |
| 885; 886; 27/28 | | | | | |
| 444; 482849861; | 1590 | 2689 | 3985 | n/a | n/a |
| NZ_AKBU01000001.1; | | | | | |
| 887; 888; 3/4 | | | | | |
| 445; 671546962; | n/a | 3131 | n/a | n/a | n/a |
| NZ_KL370786.1; 889; | | | | | |
| 890; 33/34 | | | | | |
| 446; 652698054; | 1346 | 2379 | 3788 | n/a | n/a |
| NZ_KI912610.1; 891; | | | | | |
| 892; 26/27 | | | | | |
| 447; 808064534; | 2088 | 3445 | 4433 | n/a | n/a |
| NZ_KQ040798.1; 893; | | | | | |
| 894; 17/18 | | | | | |
| 448; 808051893; | 2088 | 3445 | 4433 | n/a | n/a |
| NZ_KQ040793.1; 895; | | | | | |
| 896; 17/18 | | | | | |
| 449; 808051893; | 2088 | 3445 | 4433 | n/a | n/a |
| NZ_KQ040793.1; 897; | | | | | |
| 898; 10/11 | | | | | |
| 450; 808051893; | 2088 | 3445 | 4433 | n/a | n/a |
| NZ_KQ040793.1; 899; | | | | | |
| 900; 11/12 | | | | | |
| 451; 484016872; | n/a | 2828 | n/a | n/a | n/a |
| NZ_ANAY01000016.1; | | | | | |
| 901; 902; 27/28 | | | | | |
| 452; 736629899; | n/a | 3185 | 4322 | n/a | n/a |
| NZ_JOTN01000004.1; | | | | | |
| 903; 904; 19/20 | | | | | |
| 453; 483219562; | 1698 | 2850 | 4104 | n/a | n/a |
| NZ_KB901875.1; 905; | | | | | |
| 906; 43/44 | | | | | |
| 454; 375307420; | 1542 | 2632 | 3945 | n/a | n/a |
| NZ_JH601049.1; 907; | | | | | |
| 908; 20/21 | | | | | |
| 455; 664540649; | 1898 | 3124 | 4293 | n/a | n/a |
| NZ_JOAX01000009.1; | | | | | |
| 909; 910; 21/22 | | | | | |
| 456; 765315585; | 2075 | 3417 | 4417 | n/a | n/a |
| NZ_LN812103.1; 911; | | | | | |
| 912; 27/28 | | | | | |
| 457; 765315585; | 2075 | 3417 | 4417 | n/a | n/a |
| NZ_LN812103.1; 913; | | | | | |
| 914; 19/20 | | | | | |
| 458; 484099183; | 1771 | 2976 | 4179 | n/a | n/a |
| NZ_AJTY01001072.1; | | | | | |
| 915; 916; 19/20 | | | | | |
| 459; 647274605; | 1752 | 2948 | 4164 | n/a | n/a |
| NZ_ASSA01000134.1; | | | | | |
| 917; 918; 20/21 | | | | | |
| 460; 970574347; | 1770 | 2974 | 4008 | n/a | n/a |
| NZ_LNZF01000001.1; | | | | | |
| 919; 920; 20/21 | | | | | |
| 461; 970574347; | 1610 | 2717 | 4008 | n/a | n/a |
| NZ_LNZF01000001.1; | | | | | |
| 921; 922; 20/21 | | | | | |
| 462; 749188513; | 2012 | 3318 | 4505 | n/a | n/a |
| NZ_CP009122.1; 923; | | | | | |
| 924; 25/26 | | | | | |
| 463; 749188513; | 2012 | 3318 | 4505 | n/a | n/a |
| NZ_CP009122.1; 925; | | | | | |
| 926; 19/20 | | | | | |
| 464; 647269417; | n/a | 2977 | 4180 | n/a | n/a |
| NZ_ASSB01000031.1; | | | | | |
| 927; 928; 20/21 | | | | | |
| 465; 749188513; | 1350 | 2382 | 3789 | n/a | n/a |
| NZ_CP009122.1; 929; | | | | | |
| 930; 25/26 | | | | | |
| 466; 749188513; | 1350 | 2382 | 3789 | n/a | n/a |
| NZ_CP009122.1; 931; | | | | | |
| 932; 19/20 | | | | | |
| 467; 746717390; | n/a | 3321 | n/a | n/a | n/a |
| NZ_JSEF01000015.1; | | | | | |
| 933; 934; 16/17 | | | | | |
| 468; 738760618; | 1966 | 3221 | 4503 | n/a | n/a |
| NZ_JQCR01000002.1; | | | | | |
| 935; 936; 19/20 | | | | | |
| 469; 647230448; | n/a | 2975 | 4178 | n/a | n/a |
| NZ_ASRY01000102.1; | | | | | |
| 937; 938; 20/21 | | | | | |
| 470; 485067426; | 1714 | 2869 | 4114 | n/a | n/a |
| NZ_KB235914.1; 939; | | | | | |
| 940; 26/27 | | | | | |
| 471; 378759075; | 1522 | 3498 | 3929 | n/a | n/a |
| NZ_AFXE01000029.1; | | | | | |
| 941; 942; 22/23 | | | | | |
| 472; 924434005; | 1840 | 3071 | 4238 | n/a | n/a |
| LIYK01000027.1; 943; | | | | | |
| 944; 20/21 | | | | | |
| 473; 647274605; | 1772 | 2978 | 4181 | n/a | n/a |
| NZ_ASSA01000134.1; | | | | | |
| 945; 946; 20/21 | | | | | |
| 474; 152991597; | 1594 | 2693 | 3989 | n/a | n/a |
| NC_009663.1; 947; 948; | | | | | |
| 36/37 | | | | | |
| 475; 647274605; | 2064 | 2716 | 4007 | n/a | n/a |
| NZ_ASSA01000134.1; | | | | | |
| 949; 950; 20/21 | | | | | |
| 476; 751292755; | n/a | 3341 | 4381 | n/a | n/a |
| NZ_CCNE01000004.1; | | | | | |
| 951; 952; 26/27 | | | | | |
| 477; 256419057; | 1602 | 2702 | 3995 | n/a | n/a |
| NC_013132.1; 953; 954; | | | | | |
| 27/28 | | | | | |
| 478; 256419057; | 1602 | 2702 | 3995 | n/a | n/a |
| NC_013132.1; 955; 956; | | | | | |
| 27/28 | | | | | |
| 479; 806905234; | 2236 | 3443 | 4432 | n/a | n/a |
| NZ_LARW01000040.1; | | | | | |
| 957; 958; 11/12 | | | | | |
| 480; 663372343; | 1860 | 3086 | 4256 | n/a | n/a |
| NZ_JOFL01000022.1; | | | | | |
| 959; 960; 44/45 | | | | | |
| 481; 808064534; | 2089 | 3622 | 4434 | n/a | n/a |
| NZ_KQ040798.1; 961; | | | | | |
| 962; 10/11 | | | | | |
| 482; 808064534; | 2089 | 3622 | 4434 | n/a | n/a |
| NZ_KQ040798.1; 963; | | | | | |
| 964; 17/18 | | | | | |
| 483; 808064534; | 2089 | 3622 | 4434 | n/a | n/a |
| NZ_KQ040798.1; 965; | | | | | |
| 966; 10/11 | | | | | |
| 484; 808064534; | 2089 | 3622 | 4434 | n/a | n/a |
| NZ_KQ040798.1; 967; | | | | | |
| 968; 17/18 | | | | | |
| 485; 566226100; | 1422 | 2477 | 3853 | n/a | n/a |
| AZLX01000058.1; 969; | | | | | |
| 970; 27/28 | | | | | |
| 486; 662097244; | 1846 | 3078 | 4244 | n/a | n/a |
| NZ_KL575165.1; 971; | | | | | |
| 972; 20/21 | | | | | |
| 487; 647274605; | 1823 | 3045 | 4181 | n/a | n/a |
| NZ_ASSA01000134.1; | | | | | |
| 973; 974; 20/21 | | | | | |
| 488; 924434005; | 2000 | 3306 | 4366 | n/a | n/a |
| LIYK01000027.1; 975; | | | | | |
| 976; 20/21 | | | | | |
| 489; 378759075; | 1522 | 2609 | 3929 | n/a | n/a |
| NZ_AFXE01000029.1; | | | | | |
| 977; 978; 22/23 | | | | | |
| 490; 647274605; | 1752 | 3637 | 4520 | n/a | n/a |
| NZ_ASSA01000134.1; | | | | | |
| 979; 980; 20/21 | | | | | |
| 491; 751299847; | n/a | 3344 | 4381 | n/a | n/a |
| NZ_CCMZ01000015.1; | | | | | |
| 981; 982; 26/27 | | | | | |
| 492; 375307420; | 1576 | 2665 | 3967 | n/a | n/a |
| NZ_JH601049.1; 983; | | | | | |
| 984; 20/21 | | | | | |
| 493; 906344334; | 2131 | 3512 | 4470 | n/a | n/a |
| NZ_LFXA01000002.1; | | | | | |
| 985; 986; 25/26 | | | | | |
| 494; 759948103; | 2063 | 3611 | 4412 | n/a | n/a |
| NZ_JOAG01000045.1; | | | | | |
| 987; 988; 27/28 | | | | | |
| 495; 664478668; | 1895 | 3119 | 4288 | n/a | n/a |
| NZ_JOJI01000002.1; | | | | | |
| 989; 990; 19/20 | | | | | |
| 496; 662043624; | n/a | 3264 | 4241 | n/a | n/a |
| NZ_JNXL01000469.1; | | | | | |
| 991; 992; 22/23 | | | | | |
| 497; 906344334; | 1458 | 2528 | 3874 | n/a | n/a |
| NZ_LFXA01000002.1; | | | | | |
| 993; 994; 25/26 | | | | | |
| 498; 664104387; | 1879 | 3102 | 3924 | n/a | n/a |
| NZ_JOJJ01000005.1; | | | | | |
| 995; 996; 19/20 | | | | | |
| 499; 664104387; | 1862 | 3089 | 4258 | n/a | n/a |
| NZ_JOJJ01000005.1; | | | | | |
| 997; 998; 19/20 | | | | | |
| 500; 664104387; | 1880 | 3104 | 4274 | n/a | n/a |
| NZ_JOJJ01000005.1; | | | | | |
| 999; 1000; 19/20 | | | | | |
| 501; 664565137; | 1900 | 3605 | 4511 | n/a | n/a |
| NZ_KL591029.1; 1001; | | | | | |
| 1002; 19/20 | | | | | |
| 502; 664104387; | 1466 | 2537 | 3881 | n/a | n/a |
| NZ_JOJJ01000005.1; | | | | | |
| 1003; 1004; 19/20 | | | | | |
| 503; 664104387; | 1462 | 2533 | 3877 | n/a | n/a |
| NZ_JOJJ01000005.1; | | | | | |
| 1005; 1006; 19/20 | | | | | |
| 504; 664104387; | 1515 | 3669 | 3924 | n/a | n/a |
| NZ_JOJJ01000005.1; | | | | | |
| 1007; 1008; 19/20 | | | | | |
| 505; 664104387; | 1515 | 3161 | 4307 | n/a | n/a |
| NZ_JOJJ01000005.1; | | | | | |
| 1009; 1010; 19/20 | | | | | |
| 506; 664104387; | 1515 | 2600 | 3924 | n/a | n/a |
| NZ_JOJJ01000005.1; | | | | | |
| 1011; 1012; 19/20 | | | | | |
| 507; 664323078; | 1891 | 3111 | 4283 | n/a | n/a |
| NZ_JOIB01000032.1; | | | | | |
| 1013; 1014; 19/20 | | | | | |
| 508; 315499382; | 2137 | 2723 | n/a | n/a | n/a |
| NC_014817.1; 1015; | | | | | |
| 1016; 25/26 | | | | | |
| 509; 315499382; | 2137 | 2723 | n/a | n/a | n/a |
| NC_014817.1; 1017; | | | | | |
| 1018; 25/26 | | | | | |
| 510; 664066234; | 2263 | 3658 | 4272 | n/a | n/a |
| NZ_JOES01000124.1; | | | | | |
| 1019; 1020; 19/20 | | | | | |
| 511; 740092143; | n/a | 3585 | 4358 | n/a | n/a |
| NZ_JFCB01000064.1; | | | | | |
| 1021; 1022; 19/20 | | | | | |
| 512; 930029075; | 2276 | 3677 | n/a | n/a | n/a |
| NZ_LJHO01000007.1; | | | | | |
| 1023; 1024; 18/19 | | | | | |
| 513; 664104387; | 1515 | 3100 | 4273 | n/a | n/a |
| NZ_JOJJ01000005.1; | | | | | |
| 1025; 1026; 19/20 | | | | | |
| 514; 664104387; | 1515 | 3127 | 4258 | n/a | n/a |
| NZ_JOJJ01000005.1; | | | | | |
| 1027; 1028; 19/20 | | | | | |
| 515; 664104387; | 1464 | 2535 | 3879 | n/a | n/a |
| NZ_JOJJ01000005.1; | | | | | |
| 1029; 1030; 19/20 | | | | | |
| 516; 902792184; | n/a | 3511 | 4469 | n/a | n/a |
| NZ_LFVW01000692.1; | | | | | |
| 1031; 1032; 22/23 | | | | | |
| 517; 485125031; | 2161 | 3553 | 4378 | n/a | n/a |
| NZ_BAGL01000055.1; | | | | | |
| 1033; 1034; 18/19 | | | | | |
| 518; 759934284; | 2223 | 3607 | 4410 | n/a | n/a |
| NZ_JOAG01000009.1; | | | | | |
| 1035; 1036; 23/24 | | | | | |
| 519; 759934284; | 2223 | 3607 | 4410 | n/a | n/a |
| NZ_JOAG01000009.1; | | | | | |
| 1037; 1038; 23/24 | | | | | |
| 520; 746288194; | 2004 | 3310 | n/a | n/a | n/a |
| NZ_JRVC01000013.1; | | | | | |
| 1039; 1040; 22/23 | | | | | |
| 521; 664194528; | n/a | 2389 | n/a | n/a | n/a |
| NZ_JOIG01000002.1; | | | | | |
| 1041; 1042; 23/24 | | | | | |
| 522; 664194528; | n/a | 3455 | n/a | n/a | n/a |
| NZ_JOIG01000002.1; | | | | | |
| 1043; 1044; 23/24 | | | | | |
| 523; 664066234; | 1877 | 3099 | 4272 | n/a | n/a |
| NZ_JOES01000124.1; | | | | | |
| 1045; 1046; 19/20 | | | | | |
| 524; 664066234; | 1468 | 2539 | 3883 | n/a | n/a |
| NZ_JOES01000124.1; | | | | | |
| 1047; 1048; 19/20 | | | | | |
| 525; 72160406; | 1584 | 2676 | 3975 | n/a | n/a |
| NC_007333.1; 1049; | | | | | |
| 1050; 22/23 | | | | | |
| 526; 926371520; | n/a | 3657 | 4528 | n/a | n/a |
| NZ_LGCW01000274.1; | | | | | |
| 1051; 1052; 27128 | | | | | |
| 527; 664244706; | 1887 | 3577 | 4278 | n/a | n/a |
| NZ_JOBD01000002.1; | | | | | |
| 1053; 1054; 27/28 | | | | | |
| 528; 739594477; | 1973 | 3236 | n/a | n/a | n/a |
| NZ_JFHR01000025.1; | | | | | |
| 1055; 1056; 22/23 | | | | | |
| 529; 808402906; | 1376 | 2422 | n/a | n/a | n/a |
| CCBH010000144.1; | | | | | |
| 1057; 1058; 23/24 | | | | | |
| 530; 746242072; | 2217 | 3308 | n/a | n/a | n/a |
| NZ_JTDI01000011.1; | | | | | |
| 1059; 1060; 23/24 | | | | | |
| 531; 72160406; | 1584 | 2790 | 3975 | n/a | n/a |
| NC_007333.1; 1061; | | | | | |
| 1062; 22/23 | | | | | |
| 532; 664194528; | n/a | 3106 | n/a | n/a | n/a |
| NZ_JOIG01000002.1; | | | | | |
| 1063; 1064; 23/24 | | | | | |
| 533; 483527356; | 1709 | 2863 | n/a | n/a | n/a |
| NZ_BARE01000016.1; | | | | | |
| 1065; 1066; 22/23 | | | | | |
| 534; 936191447; | n/a | 3687 | n/a | n/a | n/a |
| NZ_LBLZ01000002.1; | | | | | |
| 1067; 1068; 22/23 | | | | | |
| 535; 484226753; | 1692 | 2843 | n/a | n/a | n/a |
| NZ_AQWM01000013.1; | | | | | |
| 1069; 1070; 21/22 | | | | | |
| 536; 664104387; | 1465 | 2536 | 3880 | n/a | n/a |
| NZ_JOJJ01000005.1; | | | | | |
| 1071; 1072; 19/20 | | | | | |
| 537; 484227180; | 1694 | 2845 | 4101 | n/a | n/a |
| NZ_AQWO01000002.1; | | | | | |
| 1073; 1074; 18/19 | | | | | |
| 538; 664104387; | 1515 | 3667 | 3924 | n/a | n/a |
| NZ_JOJJ01000005.1; | | | | | |
| 1075; 1076; 19/20 | | | | | |
| 539; 936191447; | n/a | 2399 | n/a | n/a | n/a |
| NZ_LBLZ01000002.1; | | | | | |
| 1077; 1078; 22/23 | | | | | |
| 540; 484113405; | 1730 | 2895 | n/a | n/a | n/a |
| NZ_BACX01000237.1; | | | | | |
| 1079; 1080; 23/24 | | | | | |
| 541; 664063830; | 1990 | 3571 | 4497 | n/a | n/a |
| NZ_JODT01000002.1; | | | | | |
| 1081; 1082; 28/29 | | | | | |
| 542; 451338568; | 1530 | 2617 | 3932 | n/a | n/a |
| NZ_ANMG01000060.1; | | | | | |
| 1083; 1084; 18/19 | | | | | |
| 543; 544819688; | 1728 | 2892 | n/a | n/a | n/a |
| NZ_ATHL01000147.1; | | | | | |
| 1085; 1086; 18/19 | | | | | |
| 544; 557833377; | 1742 | 2910 | n/a | n/a | n/a |
| NZ_AWGE01000008.1; | | | | | |
| 1087; 1088; 20/21 | | | | | |
| 545; 557833377; | 1742 | 2910 | n/a | n/a | n/a |
| NZ_AWGE01000008.1; | | | | | |
| 1089; 1090; 22/23 | | | | | |
| 546; 347526385; | 1625 | 2743 | n/a | n/a | n/a |
| NC_015976.1; 1091; | | | | | |
| 1092; 21/22 | | | | | |
| 547; 334133217; | 2031 | 2732 | n/a | n/a | n/a |
| NC_015579.1; 1093; | | | | | |
| 1094; 23/24 | | | | | |
| 548; 746241774; | 2002 | 3594 | n/a | n/a | n/a |
| NZ_JTDI01000009.1; | | | | | |
| 1095; 1096; 24/25 | | | | | |
| 549; 659864921; | 1843 | 3074 | n/a | n/a | n/a |
| NZ_JONW01000006.1; | | | | | |
| 1097; 1098; 20/21 | | | | | |
| 550; 659864921; | 1843 | 3074 | n/a | n/a | n/a |
| NZ_JONW01000006.1; | | | | | |
| 1099; 1100;20/21 | | | | | |
| 551; 294023656; | 1608 | 2709 | n/a | n/a | n/a |
| NC_014007.1; 1101; | | | | | |
| 1102; 23/24 | | | | | |
| 552; 749321911; | 1765 | 2966 | n/a | n/a | n/a |
| NZ_CP006644.1; 1103; | | | | | |
| 1104; 18/19 | | | | | |
| 553; 739630357; | 1977 | 3559 | n/a | n/a | n/a |
| NZ_JFYY01000027.1; | | | | | |
| 1105; 1106; 21/22 | | | | | |
| 554; 739622900; | 1975 | 3240 | n/a | n/a | n/a |
| NZ_JPPQ01000069.1; | | | | | |
| 1107; 1108; 12/13 | | | | | |
| 555; 663365281; | n/a | 3589 | 4255 | n/a | n/a |
| NZ_JODN01000094.1; | | | | | |
| 1109; 1110; 22/23 | | | | | |
| 556; 484226810; | 1693 | 2844 | n/a | n/a | n/a |
| NZ_AQWM01000032.1; | | | | | |
| 1111; 1112; 24/25 | | | | | |
| 557; 759429528; | 2177 | 3387 | n/a | n/a | n/a |
| NZ_JEMV01000036.1; | | | | | |
| 1113; 1114; 23/24 | | | | | |
| 558; 654975403; | 2173 | 3043 | 4486 | n/a | n/a |
| NZ_KI601366.1; 1115; | | | | | |
| 1116; 27/28 | | | | | |
| 559; 541476958; | 1729 | 3334 | 4375 | n/a | n/a |
| AWSB01000006.1; | | | | | |
| 1117; 1118; 58/59 | | | | | |
| 560; 484207511; | 1720 | 2879 | 4125 | n/a | n/a |
| NZ_AQUZ01000008.1; | | | | | |
| 1119; 1120; 20/21 | | | | | |
| 561; 484867900; | n/a | 2864 | n/a | n/a | n/a |
| NZ_AGNH01000612.1; | | | | | |
| 1121; 1122; 15/16 | | | | | |
| 562; 544811486; | 1908 | 2891 | n/a | n/a | n/a |
| NZ_ATDP01000107.1; | | | | | |
| 1123; 1124; 17/18 | | | | | |
| 563; 783211546; | 2085 | 3439 | 4428 | n/a | n/a |
| NZ_JZKH01000064.1; | | | | | |
| 1125; 1126; 30/31 | | | | | |
| 564; 873296042; | 2116 | 3488 | n/a | n/a | n/a |
| NZ_LECE01000021.1; | | | | | |
| 1127; 1128; 14/15 | | | | | |
| 565; 651281457; | 1937 | 3557 | 4489 | n/a | n/a |
| NZ_JADG01000010.1; | | | | | |
| 1129; 1130; 20/21 | | | | | |
| 566; 664348063; | n/a | 3495 | 4465 | n/a | n/a |
| NZ_JOFN01000002.1; | | | | | |
| 1131; 1132; 29/30 | | | | | |
| 567; 893711343; | 2123 | 3246 | n/a | n/a | n/a |
| NZ_KQ235994.1; 1133; | | | | | |
| 1134; 12/13 | | | | | |
| 568; 893711343; | 2123 | 3499 | n/a | n/a | n/a |
| NZ_KQ235994.1; 1135; | | | | | |
| 1136; 12/13 | | | | | |
| 569; 663365281; | n/a | 3576 | 4255 | n/a | n/a |
| NZ_JODN01000094.1; | | | | | |
| 1137; 1138; 22/23 | | | | | |
| 570; 739661773; | 1980 | 3587 | n/a | n/a | n/a |
| NZ_JGVR01000002.1; | | | | | |
| 1139; 1140; 13/14 | | | | | |
| 571; 739661773; | 1978 | 2608 | n/a | n/a | n/a |
| NZ_JGVR01000002.1; | | | | | |
| 1141; 1142; 13/14 | | | | | |
| 572; 749188513; | 1349 | 2381 | n/a | n/a | n/a |
| NZ_CP009122.1; 1143; | | | | | |
| 1144; 23/24 | | | | | |
| 573; 734983422; | 1932 | 3181 | n/a | n/a | n/a |
| NZ_JSX101000079.1; | | | | | |
| 1145; 1146; 18/19 | | | | | |
| 574; 930029077; | 2277 | 3678 | n/a | n/a | n/a |
| NZ_LJHO01000009.1; | | | | | |
| 1147; 1148; 22/23 | | | | | |
| 575; 664556736; | 1899 | 3604 | 4294 | n/a | n/a |
| NZ_KL591003.1; 1149; | | | | | |
| 1150; 40/41 | | | | | |
| 576; 739701660; | 1984 | 3249 | n/a | n/a | n/a |
| NZ_JNFC01000024.1; | | | | | |
| 1151; 1152; 20/21 | | | | | |
| 577; 737322991; | 2200 | 3195 | n/a | n/a | n/a |
| NZ_JMQR01000005.1; | | | | | |
| 1153; 1154; 20/21 | | | | | |
| 578; 737322991; | 2200 | 3195 | n/a | n/a | n/a |
| NZ_JMQR01000005.1; | | | | | |
| 1155; 1156; 20/21 | | | | | |
| 579; 557839256; | 1744 | 2912 | n/a | n/a | n/a |
| NZ_AWGF01000005.1; | | | | | |
| 1157; 1158; 24/25 | | | | | |
| 580; 737322991; | 1437 | 2499 | n/a | n/a | n/a |
| NZ_JMQR01000005.1; | | | | | |
| 1159; 1160; 20/21 | | | | | |
| 581; 737322991; | 1437 | 2499 | n/a | n/a | n/a |
| NZ_JMQR01000005.1; | | | | | |
| 1161; 1162; 20/21 | | | | | |
| 582; 783211546; | 2086 | 3621 | 4429 | n/a | n/a |
| NZ_JZKH01000064.1; | | | | | |
| 1163; 1164; 30/31 | | | | | |
| 583; 893711364; | 2124 | 3500 | n/a | n/a | n/a |
| NZ_KQ236015.1; 1165; | | | | | |
| 1166; 21/22 | | | | | |
| 584; 543418148; | 1429 | 2487 | n/a | n/a | n/a |
| BATC01000005.1; | | | | | |
| 1167; 1168; 26/27 | | | | | |
| 585; 797049078; | 2269 | 3666 | 4536 | n/a | n/a |
| JZWX01001028.1; | | | | | |
| 1169; 1170; 25/26 | | | | | |
| 586; 893711364; | 1979 | 3244 | n/a | n/a | n/a |
| NZ_KQ236015.1; 1171; | | | | | |
| 1172; 21/22 | | | | | |
| 587; 327367349; | 1335 | 2361 | n/a | n/a | n/a |
| CP002599.1; 1173; | | | | | |
| 1174; 27/28 | | | | | |
| 588; 494022722; | 1539 | 3242 | n/a | n/a | n/a |
| NZ_CAVK010000217.1; | | | | | |
| 1175; 1176; 21/22 | | | | | |
| 589; 893711343; | 1457 | 2527 | n/a | n/a | n/a |
| NZ_KQ235994.1; 1177; | | | | | |
| 1178; 12/13 | | | | | |
| 590; 930473294; | 2278 | 3680 | 4540 | n/a | n/a |
| NZ_LJCV01000275.1; | | | | | |
| 1179; 1180; 36/37 | | | | | |
| 591; 514419386; | 1827 | 2894 | n/a | n/a | n/a |
| NZ_KE148338.1; 1181; | | | | | |
| 1182; 22/23 | | | | | |
| 592; 930473294; | 1472 | 2546 | 3888 | n/a | n/a |
| NZ_LJCV01000275.1; | | | | | |
| 1183; 1184; 36/37 | | | | | |
| 593; 893711364; | 1521 | 2607 | n/a | n/a | n/a |
| NZ_KQ236015.1; 1185; | | | | | |
| 1186; 21/22 | | | | | |
| 594; 483682977; | 1700 | 2852 | 4483 | n/a | n/a |
| NZ_KB904636.1; 1187; | | | | | |
| 1188; 29/30 | | | | | |
| 595; 893711364; | 1546 | 2637 | n/a | n/a | n/a |
| NZ_KQ236015.1; 1189; | | | | | |
| 1190; 21/22 | | | | | |
| 596; 914607448; | 2148 | 3539 | n/a | n/a | n/a |
| NZ_JYNE01000028.1; | | | | | |
| 1191; 1192;22/23 | | | | | |
| 597; 753809381; | n/a | 2967 | n/a | n/a | n/a |
| NZ_CP006850.1; 1193; | | | | | |
| 1194; 23/24 | | | | | |
| 598; 759941310; | n/a | n/a | n/a | 3608 | n/a |
| NZ_JOAG01000020.1; | | | | | |
| 1195; 1196; 30/31 | | | | | |
| 599; 484023808; | n/a | 2833 | 4092 | n/a | n/a |
| NZ_ANBF01000204.1; | | | | | |
| 1197; 1198; 22/23 | | | | | |
| 600; 763095630; | 2067 | 3405 | n/a | n/a | n/a |
| NZ_JXZE01000009.1; | | | | | |
| 1199; 1200; 23/24 | | | | | |
| 601; 797049078; | 1471 | 2543 | 3886 | n/a | n/a |
| JZWX01001028.1; | | | | | |
| 1201; 1202; 25/26 | | | | | |
| 602; 663818579; | 1867 | 3095 | n/a | n/a | n/a |
| NZ_JNAC01000042.1; | | | | | |
| 1203; 1204; 23/24 | | | | | |
| 603; 541476958; | 1414 | 2468 | 3846 | n/a | n/a |
| AWSB01000006.1; | | | | | |
| 1205; 1206; 58/59 | | | | | |
| 604; 663300941; | 1857 | 3083 | 4253 | n/a | n/a |
| NZ_JNZY01000037.1; | | | | | |
| 1207; 1208; 25/26 | | | | | |
| 605; 196476886; | 1325 | 2350 | n/a | n/a | n/a |
| CP000747.1; 1209; | | | | | |
| 1210; 23/24 | | | | | |
| 606; 797049078; | 1455 | 2524 | 3872 | n/a | n/a |
| JZWX01001028.1; | | | | | |
| 1211; 1212; 25/26 | | | | | |
| 607; 402821166; | 1555 | 2645 | n/a | n/a | n/a |
| NZ_ALVC01000003.1; | | | | | |
| 1213; 1214; 23/24 | | | | | |
| 608; 763095630; | 1451 | 2515 | n/a | n/a | n/a |
| NZ_JXZE01000009.1; | | | | | |
| 1215; 1216; 23/24 | | | | | |
| 609; 483996974; | 1675 | 2817 | n/a | n/a | n/a |
| NZ_AMYX01000026.1; | | | | | |
| 1217; 1218; 21122 | | | | | |
| 610; 759944490; | 2062 | 3610 | 4411 | n/a | n/a |
| NZ_JOAG01000030.1; | | | | | |
| 1219; 1220; 26/27 | | | | | |
| 611; 269095543; | 1327 | 2352 | 3764 | n/a | n/a |
| CP001819.1; 1221; | | | | | |
| 1222; 13/14 | | | | | |
| 612; 393773868; | 2060 | 2647 | n/a | n/a | n/a |
| NZ_AKFJ01000097.1; | | | | | |
| 1223; 1224; 18/19 | | | | | |
| 613; 765344939; | 1982 | 2657 | n/a | n/a | n/a |
| NZ_CP010954.1; 1225; | | | | | |
| 1226; 22/23 | | | | | |
| 614; 873296295; | n/a | 3490 | n/a | n/a | n/a |
| NZ_LECE01000071.1; | | | | | |
| 1227; 1228; 23/24 | | | | | |
| 615; 759431957; | 2053 | 3388 | n/a | n/a | n/a |
| NZ_JEMV01000094.1; | | | | | |
| 1229; 1230; 12/13 | | | | | |
| 616; 765344939; | 2076 | 3421 | n/a | n/a | n/a |
| NZ_CP010954.1; 1231; | | | | | |
| 1232; 22/23 | | | | | |
| 617; 262193326; | 1603 | 2703 | n/a | n/a | n/a |
| NC_013440.1; 1233; | | | | | |
| 1234; 24/25 | | | | | |
| 618; 329889017; | 1508 | 2591 | n/a | n/a | n/a |
| NZ_GL883086.1; 1235; | | | | | |
| 1236; 19/20 | | | | | |
| 619; 664428976; | 1854 | 3116 | 4250 | n/a | n/a |
| NZ_KL585179.1; 1237; | | | | | |
| 1238; 21/22 | | | | | |
| 620; 764364074; | 2230 | 3407 | n/a | n/a | n/a |
| NZ_CP010836.1; 1239; | | | | | |
| 1240; 22/23 | | | | | |
| 621; 764364074; | 2230 | 3407 | n/a | n/a | n/a |
| NZ_CP010836.1; 1241; | | | | | |
| 1242; 19/20 | | | | | |
| 622; 402821307; | 2183 | 3219 | n/a | n/a | n/a |
| NZ_ALVC01000008.1; | | | | | |
| 1243; 1244; 12/13 | | | | | |
| 623; 484115568; | 1775 | 2985 | n/a | n/a | n/a |
| NZ_BACX01000797.1; | | | | | |
| 1245; 1246; 22/23 | | | | | |
| 624; 402821307; | 1556 | 2646 | n/a | n/a | n/a |
| NZ_ALVC01000008.1; | | | | | |
| 1247; 1248; 12/13 | | | | | |
| 625; 386845069; | 1633 | 3599 | 4037 | n/a | n/a |
| NC_017803.1; 1249; | | | | | |
| 1250; 22/23 | | | | | |
| 626; 386845069; | 1339 | 2366 | 3773 | n/a | n/a |
| NC_017803.1; 1251; | | | | | |
| 1252; 22/23 | | | | | |
| 627; 347526385; | n/a | 2742 | n/a | n/a | n/a |
| NC_015976.1; 1253; | | | | | |
| 1254; 12/13 | | | | | |
| 628; 696542396; | 2207 | 3163 | n/a | n/a | n/a |
| NZ_JQH01000002.1; | | | | | |
| 1255; 1256; 20/21 | | | | | |
| 629; 702914619; | 1926 | 3168 | 4312 | n/a | n/a |
| NZ_JNXI01000006.1; | | | | | |
| 1257; 1258; 25/26 | | | | | |
| 630; 602262270; | 1427 | 2484 | 3857 | n/a | n/a |
| JENI01000029.1; 1259; | | | | | |
| 1260; 21/22 | | | | | |
| 631; 739629085; | 1976 | 3241 | n/a | n/a | n/a |
| NZ_JFYY01000016.1; | | | | | |
| 1261; 1262; 23/24 | | | | | |
| 632; 602262270; | 1956 | 3213 | 3980 | n/a | n/a |
| JENI01000029.1; 1263; | | | | | |
| 1264; 21/22 | | | | | |
| 633; 602262270; | n/a | 2683 | 3980 | n/a | n/a |
| JENI01000029.1; 1265; | | | | | |
| 1266; 21/22 | | | | | |
| 634; 602262270; | 1421 | 2476 | 3852 | n/a | n/a |
| JENI01000029.1; 1267; | | | | | |
| 1268; 21/22 | | | | | |
| 635; 659889283; | 1844 | 3253 | n/a | n/a | n/a |
| NZ_JOOE01000001.1; | | | | | |
| 1269; 1270; 18/19 | | | | | |
| 636; 737322991; | 2201 | 3196 | n/a | n/a | n/a |
| NZ_JMQR01000005.1; | | | | | |
| 1271; 1272; 19/20 | | | | | |
| 637; 444405902; | 1509 | 2592 | n/a | n/a | n/a |
| NZ_KB291784.1; 1273; | | | | | |
| 1274; 20/21 | | | | | |
| 638; 444405902; | 1509 | 2592 | n/a | n/a | n/a |
| NZ_KB291784.1; 1275; | | | | | |
| 1276; 20/21 | | | | | |
| 639; 602262270; | 1956 | 3210 | 3980 | n/a | n/a |
| JENI01000029.1; 1277; | | | | | |
| 1278; 21/22 | | | | | |
| 640; 546154317; | 1415 | 2469 | 3847 | n/a | n/a |
| NZ_ACVN02000045.1; | | | | | |
| 1279; 1280; 18/19 | | | | | |
| 641; 602262270; | 1956 | 3212 | 4333 | n/a | n/a |
| JENI01000029.1; 1281; | | | | | |
| 1282; 21/22 | | | | | |
| 642; 938956730; | 2284 | 3693 | n/a | n/a | n/a |
| NZ_CP009429.1; 1283; | | | | | |
| 1284; 20/21 | | | | | |
| 643; 602262270; | 1439 | 2501 | 3862 | n/a | n/a |
| JENI01000029.1; 1285; | | | | | |
| 1286; 21/22 | | | | | |
| 644; 737323704; | n/a | 3197 | n/a | n/a | n/a |
| NZ_JMQR01000012.1; | | | | | |
| 1287; 1288; 19/20 | | | | | |
| 645; 737323704; | n/a | 3197 | n/a | n/a | n/a |
| NZ_JMQR01000012.1; | | | | | |
| 1289; 1290; 18/19 | | | | | |
| 646; 602262270; | 1441 | 2503 | 3863 | n/a | n/a |
| JENI01000029.1; 1291; | | | | | |
| 1292; 21/22 | | | | | |
| 647; 657605746; | 1836 | 3067 | n/a | n/a | n/a |
| NZ_JNIX01000010.1; | | | | | |
| 1293; 1294; 18/19 | | | | | |
| 648; 647728918; | 1774 | 2980 | n/a | n/a | n/a |
| NZ_JHOF01000018.1; | | | | | |
| 1295; 1296; 19/20 | | | | | |
| 649; 938989745; | 2288 | 3697 | n/a | n/a | n/a |
| NZ_CP012897.1; 1297; | | | | | |
| 1298; 20/21 | | | | | |
| 650; 938989745; | 2288 | 3697 | n/a | n/a | n/a |
| NZ_CP012897.1; 1299; | | | | | |
| 1300; 19/20 | | | | | |
| 651; 664434000; | n/a | 3118 | n/a | n/a | n/a |
| NZ_JOIA01001078.1; | | | | | |
| 1301; 1302; 21/22 | | | | | |
| 652; 703243990; | n/a | 3588 | n/a | n/a | n/a |
| NZ_JNYM01001430.1; | | | | | |
| 1303; 1304; 20/21 | | | | | |
| 653; 739699072; | 1983 | 3248 | n/a | n/a | n/a |
| NZ_JNFC01000001.1; | | | | | |
| 1305; 1306; 19/20 | | | | | |
| 654; 739699072; | 1983 | 3248 | n/a | n/a | n/a |
| NZ_JNFC01000001.1; | | | | | |
| 1307; 1308; 19/20 | | | | | |
| 655; 739699072; | 1983 | 3319 | n/a | n/a | n/a |
| NZ_JNFC01000001.1; | | | | | |
| 1309; 1310; 19/20 | | | | | |
| 656; 739699072; | 1983 | 3319 | n/a | n/a | n/a |
| NZ_JNFC01000001.1; | | | | | |
| 1311; 1312; 19/20 | | | | | |
| 657; 343957487; | 1573 | 2662 | n/a | n/a | n/a |
| NZ_AEWF01000005.1; | | | | | |
| 1313; 1314; 31/32 | | | | | |
| 658; 343957487; | 1573 | 2662 | n/a | n/a | n/a |
| NZ_AEWF01000005.1; | | | | | |
| 1315; 1316; 31/32 | | | | | |
| 659; 938154362; | 1364 | 2401 | n/a | n/a | n/a |
| CP009430.1; 1317; | | | | | |
| 1318; 23/24 | | | | | |
| 660; 566155502; | 1746 | 2914 | 4151 | n/a | n/a |
| NZ_CM002285.1; 1319; | | | | | |
| 1320; 37/38 | | | | | |
| 661; 399903251; | n/a | 2453 | 3834 | n/a | n/a |
| ALJK01000024.1; 1321; | | | | | |
| 1322; 22/23 | | | | | |
| 662; 399903251; | n/a | 2453 | 3834 | n/a | n/a |
| ALJK01000024.1; 1323; | | | | | |
| 1324; 21/22 | | | | | |
| 663; 399903251; | n/a | 2453 | 3834 | n/a | n/a |
| ALJK01000024.1; 1325; | | | | | |
| 1326; 24/25 | | | | | |
| 664; 763097360; | 2229 | 3617 | n/a | n/a | n/a |
| NZ_JXZE01000017.1; | | | | | |
| 1327; 1328; 21/22 | | | | | |
| 665; 746290581; | 2218 | 3595 | n/a | n/a | n/a |
| NZ_JRVC01000028.1; | | | | | |
| 1329; 1330; 22/23 | | | | | |
| 666; 739287390; | 2206 | 3137 | 4303 | n/a | n/a |
| NZ_JMFA01000010.1; | | | | | |
| 1331; 1332; 21/22 | | | | | |
| 667; 694033726; | 2206 | 3137 | 4303 | n/a | n/a |
| NZ_JMEM01000016.1; | | | | | |
| 1333; 1334; 21/22 | | | | | |
| 668; 739287390; | 2206 | 3137 | 4303 | n/a | n/a |
| NZ_JMFA01000010.1; | | | | | |
| 1335; 1336; 21/22 | | | | | |
| 669; 483997957; | 1677 | 2819 | n/a | n/a | n/a |
| NZ_AMYY01000002.1; | | | | | |
| 1337; 1338; 20/21 | | | | | |
| 670; 898301838; | n/a | 3510 | n/a | n/a | n/a |
| NZ_LAVK01000307.1; | | | | | |
| 1339; 1340; 36/37 | | | | | |
| 671; 739287390; | 2205 | 3138 | 4303 | n/a | n/a |
| NZ_JMFA01000010.1; | | | | | |
| 1341; 1342; 21/22 | | | | | |
| 672; 739287390; | 2205 | 3138 | 4303 | n/a | n/a |
| NZ_JMFA01000010.1; | | | | | |
| 1343; 1344; 21/22 | | | | | |
| 673; 739287390; | 2205 | 3138 | 4303 | n/a | n/a |
| NZ_JMFA01000010.1; | | | | | |
| 1345; 1346; 21/22 | | | | | |
| 674; 739287390; | 2205 | 3230 | 4303 | n/a | n/a |
| NZ_JMFA01000010.1; | | | | | |
| 1347; 1348; 21/22 | | | | | |
| 675; 739287390; | 2205 | 3230 | 4303 | n/a | n/a |
| NZ_JMFA01000010.1; | | | | | |
| 1349; 1350; 21/22 | | | | | |
| 676; 739287390; | 2205 | 3230 | 4303 | n/a | n/a |
| NZ_JMFA01000010.1; | | | | | |
| 1351; 1352; 21/22 | | | | | |
| 677; 766589647; | 1754 | 2950 | 4166 | n/a | n/a |
| NZ_CEHJ01000007.1; | | | | | |
| 1353; 1354; 18/19 | | | | | |
| 678; 938989745; | 2289 | 3698 | n/a | n/a | n/a |
| NZ_CP012897.1; 1355; | | | | | |
| 1356; 20/21 | | | | | |
| 679; 938989745; | 2289 | 3698 | n/a | n/a | n/a |
| NZ_CP012897.1; 1357; | | | | | |
| 1358; 20/21 | | | | | |
| 680; 739610197; | 1974 | 3238 | n/a | n/a | n/a |
| NZ_EZA02000028.1; | | | | | |
| 1359; 1360; 22/23 | | | | | |
| 681; 766589647; | 2081 | 3430 | 4423 | n/a | n/a |
| NZ_CEHJ01000007.1; | | | | | |
| 1361; 1362; 18/19 | | | | | |
| 682; 896667361; | 2130 | 3509 | 4468 | n/a | n/a |
| NZ_JVGV01000030.1; | | | | | |
| 1363; 1364; 18/19 | | | | | |
| 683; 834156795; | 1435 | 2496 | n/a | n/a | n/a |
| BBRO01000001.1; | | | | | |
| 1365; 1366; 20/21 | | | | | |
| 684; 736736050; | 2184 | 3561 | n/a | n/a | n/a |
| NZ_AWFG01000029.1; | | | | | |
| 1367; 1368; 27/28 | | | | | |
| 685; 766589647; | 1754 | 3424 | 4166 | n/a | n/a |
| NZ_CEHJ01000007.1; | | | | | |
| 1369; 1370; 18/19 | | | | | |
| 686; 938956730; | 1363 | 2400 | n/a | n/a | n/a |
| NZ_CP009429.1; 1371; | | | | | |
| 1372; 19/20 | | | | | |
| 687; 938956730; | 1363 | 2400 | n/a | n/a | n/a |
| NZ_CP009429.1; 1373; | | | | | |
| 1374; 21/22 | | | | | |
| 688; 545327527; | n/a | 2893 | 4376 | n/a | n/a |
| NZ_KE951412.1; 1375; | | | | | |
| 1376; 25/26 | | | | | |
| 689; 545327527; | n/a | 2893 | 4376 | n/a | n/a |
| NZ_KE951412.1; 1377; | | | | | |
| 1378; 13/14 | | | | | |
| 690; 545327527; | n/a | 2893 | 4376 | n/a | n/a |
| NZ_KE951412.1; 1379; | | | | | |
| 1380; 19/20 | | | | | |
| 691; 545327527; | n/a | 2893 | 4376 | n/a | n/a |
| NZ_KE951412.1; 1381; | | | | | |
| 1382; 19/20 | | | | | |
| 692; 541473965; | n/a | 2893 | 4376 | n/a | n/a |
| AWSB01000041.1; | | | | | |
| 1383; 1384; 20/21 | | | | | |
| 693; 896567682; | 2128 | 3507 | n/a | n/a | n/a |
| NZ_JUMH01000022.1; | | | | | |
| 1385; 1386; 16/17 | | | | | |
| 694; 728827031; | 2210 | 3178 | n/a | n/a | n/a |
| NZ_JROG01000008.1; | | | | | |
| 1387; 1388; 20/21 | | | | | |
| 695; 896567682; | 2126 | 3502 | n/a | n/a | n/a |
| NZ_JUMH01000022.1; | | | | | |
| 1389; 1390; 16/17 | | | | | |
| 696; 896567682; | 1914 | 3136 | n/a | n/a | n/a |
| NZ_JUMH01000022.1; | | | | | |
| 1391; 1392; 16/17 | | | | | |
| 697; 387783149; | 2035 | 2752 | 4036 | n/a | n/a |
| NC_017595.1; 1393; | | | | | |
| 1394; 18/19 | | | | | |
| 698; 484021228; | 2156 | 2860 | n/a | n/a | n/a |
| NZ_KB895788.1; 1395; | | | | | |
| 1396; 21/22 | | | | | |
| 699; 269095543; | n/a | 3379 | 3997 | n/a | n/a |
| CP001819.1; 1397; | | | | | |
| 1398; 13/14 | | | | | |
| 700; 663372947; | n/a | 3087 | n/a | n/a | n/a |
| NZ_JOFL01000031.1; | | | | | |
| 1399; 1400; 32/33 | | | | | |
| 701; 692233141; | 1913 | 3135 | n/a | n/a | n/a |
| NZ_JQAK01000001.1; | | | | | |
| 1401; 1402; 24/25 | | | | | |
| 702; 692233141; | 1913 | 3135 | n/a | n/a | n/a |
| NZ_JQAK01000001.1; | | | | | |
| 1403; 1404; 24/25 | | | | | |
| 703; 896520167; | 2127 | 3504 | n/a | n/a | n/a |
| NZ_JVUI01000038.1; | | | | | |
| 1405; 1406; 16/17 | | | | | |
| 704; 194363778; | 1600 | 2699 | n/a | n/a | n/a |
| NC_011071.1; 1407; | | | | | |
| 1408; 36/37 | | | | | |
| 705; 737569369; | 1950 | 3204 | n/a | n/a | n/a |
| NZ_ARYL01000059.1; | | | | | |
| 1409; 1410; 27/28 | | | | | |
| 706; 484033611; | 1686 | 2836 | n/a | n/a | n/a |
| NZ_ANFZ01000008.1; | | | | | |
| 1411; 1412; 20/21 | | | | | |
| 707; 780834515; | n/a | 2522 | n/a | n/a | n/a |
| LADU01000087.1; | | | | | |
| 1413; 1414; 27/28 | | | | | |
| 708; 927084736; | 2268 | 3665 | 4535 | n/a | n/a |
| NZ_LITU01000056.1; | | | | | |
| 1415; 1416; 21/22 | | | | | |
| 709; 522837181; | 1406 | 2460 | 3839 | n/a | n/a |
| NZ_KE352807.1; 1417; | | | | | |
| 1418; 22/23 | | | | | |
| 710; 737569369; | 1938 | 3186 | n/a | n/a | n/a |
| NZ_ARYL01000059.1; | | | | | |
| 1419; 1420; 27/28 | | | | | |
| 711; 737577234; | 1952 | 3206 | n/a | n/a | n/a |
| NZ_AWFH01000002.1; | | | | | |
| 1421; 1422; 27/28 | | | | | |
| 712; 522837181; | 1405 | 2459 | 3838 | n/a | n/a |
| NZ_KE352807.1; 1423; | | | | | |
| 1424; 22/23 | | | | | |
| 713; 522837181; | 1505 | 2587 | 3918 | n/a | n/a |
| NZ_KE352807.1; 1425; | | | | | |
| 1426; 22/23 | | | | | |
| 714; 522837181; | 1504 | 2963 | 3918 | n/a | n/a |
| NZ_KE352807.1; 1427; | | | | | |
| 1428; 22/23 | | | | | |
| 715; 522837181; | 1410 | 2464 | 3842 | n/a | n/a |
| NZ_KE352807.1; 1429; | | | | | |
| 1430; 22/23 | | | | | |
| 716; 522837181; | n/a | 2454 | 3835 | n/a | n/a |
| NZ_KE352807.1; 1431; | | | | | |
| 1432; 22/23 | | | | | |
| 717; 522837181; | n/a | 2964 | 3918 | n/a | n/a |
| NZ_KE352807.1; 1433; | | | | | |
| 1434; 22/23 | | | | | |
| 718; 522837181; | 1763 | 2962 | 3918 | n/a | n/a |
| NZ_KE352807.1; 1435; | | | | | |
| 1436; 22/23 | | | | | |
| 719; 522837181; | 1503 | 2586 | 3918 | n/a | n/a |
| NZ_KE352807.1; 1437; | | | | | |
| 1438; 22/23 | | | | | |
| 720; 522837181; | 1372 | 2415 | 3810 | n/a | n/a |
| NZ_KE352807.1; 1439; | | | | | |
| 1440; 22/23 | | | | | |
| 721; 522837181; | n/a | 2439 | 3827 | n/a | n/a |
| NZ_KE352807.1; 1441; | | | | | |
| 1442; 22/23 | | | | | |
| 722; 822535978; | 2097 | 3462 | n/a | n/a | n/a |
| NZ_JPLE01000028.1; | | | | | |
| 1443; 1444; 35/36 | | | | | |
| 723; 924898949; | 1360 | 2395 | n/a | n/a | n/a |
| NZ_CP009452.1; 1445; | | | | | |
| 1446; 18/19 | | | | | |
| 724; 924516300; | 2252 | 3643 | n/a | n/a | n/a |
| NZ_LDVR01000003.1; | | | | | |
| 1447; 1448; 36/37 | | | | | |
| 725; 541473965; | 1413 | 2467 | 3845 | n/a | n/a |
| AWSB01000041.1; | | | | | |
| 1449; 1450; 20/21 | | | | | |
| 726; 483532492; | 1710 | n/a | n/a | n/a | n/a |
| NZ_BARE01000100.1; | | | | | |
| 1451; 1452; 19/20 | | | | | |
| 727; 655095554; | 1824 | 3224 | 4219 | n/a | n/a |
| NZ_AULE01000001.1; | | | | | |
| 1453; 1454; 22/23 | | | | | |
| 728; 541473965; | n/a | 2893 | 4376 | n/a | n/a |
| AWSB01000041.1; | | | | | |
| 1455; 1456; 20/21 | | | | | |
| 729; 545327527; | n/a | 2893 | 4376 | n/a | n/a |
| NZ_KE951412.1; 1457; | | | | | |
| 1458; 20/21 | | | | | |
| 730; 545327527; | n/a | 2893 | 4376 | n/a | n/a |
| NZ_KE951412.1; 1459; | | | | | |
| 1460; 13/14 | | | | | |
| 731; 545327527; | n/a | 2893 | 4376 | n/a | n/a |
| NZ_KE951412.1; 1461; | | | | | |
| 1462; 20/21 | | | | | |
| 732; 651445346; | n/a | 2994 | 4188 | n/a | n/a |
| NZ_AZVC01000006.1; | | | | | |
| 1463; 1464; 21/22 | | | | | |
| 733; 739650776; | 2208 | 3243 | n/a | n/a | n/a |
| NZ_KL662193.1; 1465; | | | | | |
| 1466; 29/30 | | | | | |
| 734; 260447107; | 1559 | 2651 | 3957 | n/a | n/a |
| NZ_GG703879.1; 1467; | | | | | |
| 1468; 13/14 | | | | | |
| 735; 260447107; | 1559 | 2651 | 3957 | n/a | n/a |
| NZ_GG703879.1; 1469; | | | | | |
| 1470; 20/21 | | | | | |
| 736; 260447107; | 1559 | 2651 | 3957 | n/a | n/a |
| NZ_GG703879.1; 1471; | | | | | |
| 1472; 20/21 | | | | | |
| 737; 260447107; | 1559 | 2651 | 3957 | n/a | n/a |
| NZ_GG703879.1; 1473; | | | | | |
| 1474; 20/21 | | | | | |
| 738; 260447107; | 1559 | 2651 | 3957 | n/a | n/a |
| NZ_GG703879.1; 1475; | | | | | |
| 1476; 20/21 | | | | | |
| 739; 737567115; | 1949 | 3203 | n/a | n/a | n/a |
| NZ_ARYL01000020.1; | | | | | |
| 1477; 1478; 26/27 | | | | | |
| 740; 343957487; | 1572 | 2661 | n/a | n/a | n/a |
| NZ_AEWF01000005.1; | | | | | |
| 1479; 1480; 29/30 | | | | | |
| 741; 528200987; | n/a | 3560 | 4135 | n/a | n/a |
| ATMS01000061.1; | | | | | |
| 1481; 1482; 22/23 | | | | | |
| 742; 896535166; | 1579 | 3505 | n/a | n/a | n/a |
| NZ_JVHW01000017.1; | | | | | |
| 1483; 1484; 33/34 | | | | | |
| 743; 896535166; | 2129 | 3508 | n/a | n/a | n/a |
| NZ_JVHW01000017.1; | | | | | |
| 1485; 1486; 33/34 | | | | | |
| 744; 896535166; | 1579 | 3503 | n/a | n/a | n/a |
| NZ_JVHW01000017.1; | | | | | |
| 1487; 1488; 33/34 | | | | | |
| 745; 730274767; | 2216 | 3179 | n/a | n/a | n/a |
| NZ_JSBN01000149.1; | | | | | |
| 1489; 1490; 22/23 | | | | | |
| 746; 896555871; | 1579 | 3506 | n/a | n/a | n/a |
| NZ_JVRD01000056.1; | | | | | |
| 1491; 1492; 33/34 | | | | | |
| 747; 740097110; | 1994 | 3273 | 4359 | n/a | n/a |
| NZ_JABQ01000001.1; | | | | | |
| 1493; 1494; 48/49 | | | | | |
| 748; 930169273; | 2129 | 3679 | n/a | n/a | n/a |
| NZ_LJJH01000098.1; | | | | | |
| 1495; 1496; 33/34 | | | | | |
| 749; 923067758; | 2250 | 3640 | n/a | n/a | n/a |
| NZ_CP011010.1; 1497; | | | | | |
| 1498; 33/34 | | | | | |
| 750; 484978121; | 1841 | 2866 | n/a | n/a | n/a |
| NZ_AGRB01000040.1; | | | | | |
| 1499; 1500; 33/34 | | | | | |
| 751; 664275807; | n/a | 3573 | 4280 | n/a | n/a |
| NZ_JOIX01000031.1; | | | | | |
| 1501; 1502; 39/40 | | | | | |
| 752; 737580759; | 1953 | 3207 | n/a | n/a | n/a |
| NZ_AWFH01000021.1; | | | | | |
| 1503; 1504;31/32 | | | | | |
| 753; 484978121; | 2249 | 3639 | n/a | n/a | n/a |
| NZ_AGRB01000040.1; | | | | | |
| 1505; 1506; 33/34 | | | | | |
| 754; 896535166; | 1579 | 2667 | n/a | n/a | n/a |
| NZ_JVHW01000017.1; | | | | | |
| 1507; 1508; 33/34 | | | | | |
| 755; 896535166; | 1579 | 3395 | n/a | n/a | n/a |
| NZ_JVHW01000017.1; | | | | | |
| 1509; 1510; 33/34 | | | | | |
| 756; 434402184; | 2027 | 2766 | 4386 | n/a | n/a |
| NC_019757.1; 1511; | | | | | |
| 1512; 27/28 | | | | | |
| 757; 522837181; | n/a | 2440 | 3828 | n/a | n/a |
| NZ_KE352807.1; 1513; | | | | | |
| 1514; 22/23 | | | | | |
| 758; 640451877; | 1759 | 2959 | n/a | n/a | n/a |
| NZ_AYSW01000160.1; | | | | | |
| 1515; 1516; 13/14 | | | | | |
| 759; 640451877; | 1759 | 2959 | n/a | n/a | n/a |
| NZ_AYSW01000160.1; | | | | | |
| 1517; 1518; 17/18 | | | | | |
| 760; 640451877; | 1759 | 2959 | n/a | n/a | n/a |
| NZ_AYSW01000160.1; | | | | | |
| 1519; 1520; 16/17 | | | | | |
| 761; 528200987; | 1411 | 2465 | 3843 | n/a | n/a |
| ATMS01000061.1; | | | | | |
| 1521; 1522; 22/23 | | | | | |
| 762; 780821511; | n/a | 2521 | n/a | n/a | n/a |
| LADW01000068.1; | | | | | |
| 1523; 1524; 24/25 | | | | | |
| 763; 566231608; | 1423 | 2478 | 3854 | n/a | n/a |
| AZMH01000257.1; | | | | | |
| 1525; 1526; 19/20 | | | | | |
| 764; 736764136; | 1940 | 3188 | n/a | n/a | n/a |
| NZ_AWFD01000033.1; | | | | | |
| 1527; 1528; 27/28 | | | | | |
| 765; 737608363; | 1954 | 3208 | n/a | n/a | n/a |
| NZ_ARYJ01000002.1; | | | | | |
| 1529; 1530; 17/18 | | | | | |
| 766; 145690656; | 1322 | 2344 | n/a | n/a | n/a |
| CP000408.1; 1531; | | | | | |
| 1532; 19/20 | | | | | |
| 767; 145690656; | 1322 | 2344 | n/a | n/a | n/a |
| CP000408.1; 1533; | | | | | |
| 1534; 19/20 | | | | | |
| 768; 815863894; | n/a | 3453 | 4436 | n/a | n/a |
| NZ_LAJC01000044.1; | | | | | |
| 1535; 1536; 13/14 | | | | | |
| 769; 145690656; | 1371 | 2413 | 3808 | n/a | n/a |
| CP000408.1; 1537; | | | | | |
| 1538; 19/20 | | | | | |
| 770; 145690656; | 1371 | 2413 | 3808 | n/a | n/a |
| CP000408.1; 1539; | | | | | |
| 1540; 19/20 | | | | | |
| 771; 550281965; | 1416 | 2470 | 3848 | n/a | n/a |
| NZ_ASSJ01000070.1; | | | | | |
| 1541; 1542; 27/28 | | | | | |
| 772; 484113491; | 1731 | 2896 | n/a | n/a | n/a |
| NZ_BACX01000258.1; | | | | | |
| 1543; 1544; 10/11 | | | | | |
| 773; 145690656; | 1592 | 2949 | 3994 | n/a | n/a |
| CP000408.1; 1545; | | | | | |
| 1546; 19/20 | | | | | |
| 774; 145690656; | 1592 | 2949 | 3994 | n/a | n/a |
| CP000408.1; 1547; | | | | | |
| 1548; 19/20 | | | | | |
| 775; 483258918; | 2077 | 3422 | 4419 | n/a | n/a |
| NZ_AMFE01000033.1; | | | | | |
| 1549; 1550; 19/20 | | | | | |
| 776; 483258918; | 2077 | 3422 | 4419 | n/a | n/a |
| NZ_AMFE01000033.1; | | | | | |
| 1551; 1552; 19/20 | | | | | |
| 777; 145690656; | n/a | 2345 | n/a | n/a | n/a |
| CP000408.1; 1553; | | | | | |
| 1554; 19/20 | | | | | |
| 778; 145690656; | n/a | 2345 | n/a | n/a | n/a |
| CP000408.1; 1555; | | | | | |
| 1556; 19/20 | | | | | |
| 779; 483258918; | 2078 | 3425 | 4419 | n/a | n/a |
| NZ_AMFE01000033.1; | | | | | |
| 1557; 1558; 19/20 | | | | | |
| 780; 766595491; | 2078 | 3425 | 4419 | n/a | n/a |
| NZ_CEHM01000004.1; | | | | | |
| 1559; 1560; 19/20 | | | | | |
| 781; 737951550; | 1959 | 3562 | 4334 | n/a | n/a |
| NZ_JAAG01000075.1; | | | | | |
| 1561; 1562; 19/20 | | | | | |
| 782; 879201007; | 1483 | 2557 | 3907 | n/a | n/a |
| CKIK01000005.1; 1563; | | | | | |
| 1564; 19/20 | | | | | |
| 783; 879201007; | 1484 | 3523 | 3907 | n/a | n/a |
| CKIK01000005.1; 1565; | | | | | |
| 1566; 19/20 | | | | | |
| 784; 879201007; | 1483 | 3684 | 3907 | n/a | n/a |
| CKIK01000005.1; 1567; | | | | | |
| 1568; 19/20 | | | | | |
| 785; 879201007; | 1484 | 3524 | 3907 | n/a | n/a |
| CKIK01000005.1; 1569; | | | | | |
| 1570; 19/20 | | | | | |
| 786; 879201007; | 1484 | 2558 | 3907 | n/a | n/a |
| CKIK01000005.1; 1571; | | | | | |
| 1572; 19/20 | | | | | |
| 787; 483258918; | 1671 | 2812 | 4082 | n/a | n/a |
| NZ_AMFE01000033.1; | | | | | |
| 1573; 1574; 19/20 | | | | | |
| 788; 483258918; | 1671 | 2812 | 4082 | n/a | n/a |
| NZ_AMFE01000033.1; | | | | | |
| 1575; 1576; 19/20 | | | | | |
| 789; 879201007; | 1382 | 2430 | 3822 | n/a | n/a |
| CKIK01000005.1; 1577; | | | | | |
| 1578; 19/20 | | | | | |
| 790; 950938054; | 1381 | 2429 | 3821 | n/a | n/a |
| NZ_CIHL01000007.1; | | | | | |
| 1579; 1580; 19/20 | | | | | |
| 791; 739748927; | 1986 | 3254 | 4346 | n/a | n/a |
| NZ_JJMT01000011.1; | | | | | |
| 1581; 1582; 19/20 | | | | | |
| 792; 739748927; | 1986 | 3254 | 4346 | n/a | n/a |
| NZ_JJMT01000011.1; | | | | | |
| 1583; 1584; 19/20 | | | | | |
| 793; 655069822; | 1822 | 3044 | 4218 | n/a | n/a |
| NZ_KI912489.1; 1585; | | | | | |
| 1586; 19/20 | | | | | |
| 794; 655069822; | 1822 | 3044 | 4218 | n/a | n/a |
| NZ_KI912489.1; 1587; | | | | | |
| 1588; 19/20 | | | | | |
| 795; 655069822; | 1822 | 3044 | 4218 | n/a | n/a |
| NZ_KI912489.1; 1589; | | | | | |
| 1590; 19/20 | | | | | |
| 796; 655069822; | 1822 | 3044 | 4218 | n/a | n/a |
| NZ_KI912489.1; 1591; | | | | | |
| 1592; 19/20 | | | | | |
| 797; 655069822; | 1822 | 3044 | 4218 | n/a | n/a |
| NZ_KI912489.1; 1593; | | | | | |
| 1594; 19/20 | | | | | |
| 798; 655069822; | 1822 | 3044 | 4218 | n/a | n/a |
| NZ_KI912489.1; 1595; | | | | | |
| 1596; 19/20 | | | | | |
| 799; 664428976; | 1854 | 3116 | 4250 | n/a | n/a |
| NZ_KL585179.1; 1597; | | | | | |
| 1598; 21/22 | | | | | |
| 800; 325680876; | 1393 | 2444 | 3831 | n/a | n/a |
| NZ_ADKM02000123.1; | | | | | |
| 1599; 1600; 19/20 | | | | | |
| 801; 325680876; | 1507 | 3231 | 4344 | n/a | n/a |
| NZ_ADKM02000123.1; | | | | | |
| 1601; 1602; 19/20 | | | | | |
| 802; 759443001; | n/a | 3389 | 4405 | n/a | n/a |
| NZ_JDUV01000004.1; | | | | | |
| 1603; 1604; 20/21 | | | | | |
| 803; 759443001; | n/a | 3406 | 4405 | n/a | n/a |
| NZ_JDUV01000004.1; | | | | | |
| 1605; 1606; 20/21 | | | | | |
| 804; 551695014; | 1417 | 2471 | 3849 | n/a | n/a |
| AXZG01000035.1; | | | | | |
| 1607; 1608; 18/19 | | | | | |
| 805; 551695014; | 1417 | 2471 | 3849 | n/a | n/a |
| AXZG01000035.1; | | | | | |
| 1609; 1610; 9/10 | | | | | |
| 806; 818310996; | 1456 | 2526 | n/a | n/a | n/a |
| LBRK01000013.1; | | | | | |
| 1611; 1612; 29/30 | | | | | |
| 807; 213690928; | n/a | 2700 | 3992 | n/a | n/a |
| NC_011593.1; 1613; | | | | | |
| 1614; 20/21 | | | | | |
| 808; 383809261; | 1538 | 2628 | 4343 | n/a | n/a |
| NZ_AJJQ01000036.1; | | | | | |
| 1615; 1616; 18/19 | | | | | |
| 809; 383809261; | 1538 | 2628 | 4343 | n/a | n/a |
| NZ_AJJQ01000036.1; | | | | | |
| 1617; 1618; 9/10 | | | | | |
| 810; 551695014; | 1738 | 3233 | 4146 | n/a | n/a |
| AXZG01000035.1; | | | | | |
| 1619; 1620; 18/19 | | | | | |
| 811; 551695014; | 1738 | 3233 | 4146 | n/a | n/a |
| AXZG01000035.1; | | | | | |
| 1621; 1622; 9/10 | | | | | |
| 812; 484007841; | 1679 | 2823 | 4088 | n/a | n/a |
| NZ_ANAD01000138.1; | | | | | |
| 1623; 1624; 28/29 | | | | | |
| 813; 739372122; | 2204 | 3592 | 4343 | n/a | n/a |
| NZ_JQHE01000003.1; | | | | | |
| 1625; 1626; 11/12 | | | | | |
| 814; 739372122; | 2204 | 3592 | 4343 | n/a | n/a |
| NZ_JQHE01000003.1; | | | | | |
| 1627; 1628; 13/14 | | | | | |
| 815; 357386972; | 1627 | 2745 | n/a | n/a | n/a |
| NC_016109.1; 1629; | | | | | |
| 1630; 26/27 | | | | | |
| 816; 749295448; | n/a | 2965 | 4173 | n/a | n/a |
| NZ_CP006714.1; 1631; | | | | | |
| 1632; 20/21 | | | | | |
| 817; 260447107; | 1559 | 2651 | 3957 | n/a | n/a |
| NZ_GG703879.1; 1633; | | | | | |
| 1634; 20/21 | | | | | |
| 818; 260447107; | 1559 | 2651 | 3957 | n/a | n/a |
| NZ_GG703879.1; 1635; | | | | | |
| 1636; 13/14 | | | | | |
| 819; 260447107; | 1559 | 2651 | 3957 | n/a | n/a |
| NZ_GG703879.1; 1637; | | | | | |
| 1638; 20/21 | | | | | |
| 820; 260447107; | 1559 | 2651 | 3957 | n/a | n/a |
| NZ_GG703879.1; 1639; | | | | | |
| 1640; 20/21 | | | | | |
| 821; 260447107; | 1559 | 2651 | 3957 | n/a | n/a |
| NZ_GG703879.1; 1641; | | | | | |
| 1642; 20/21 | | | | | |
| 822; 749295448; | n/a | 2397 | 3797 | n/a | n/a |
| NZ_CP006714.1; 1643; | | | | | |
| 1644; 20/21 | | | | | |
| 823; 759443001; | 1442 | n/a | n/a | 2504 | n/a |
| NZ_JDUV01000004.1; | | | | | |
| 1645; 1646; 20/21 | | | | | |
| 824; 67639376; | 1460 | 2531 | n/a | n/a | n/a |
| NZ_AAHO01000116.1; | | | | | |
| 1647; 1648; 28/29 | | | | | |
| 825; 483969755; | 1703 | 2857 | n/a | n/a | n/a |
| NZ_KB891596.1; 1649; | | | | | |
| 1650; 34/35 | | | | | |
| 826; 484026206; | 1684 | 3337 | 4094 | n/a | n/a |
| NZ_ANBH01000093.1; | | | | | |
| 1651; 1652; 31/32 | | | | | |
| 827; 919546672; | n/a | 3630 | n/a | n/a | n/a |
| NZ_JOEL01000066.1; | | | | | |
| 1653; 1654; 31/32 | | | | | |
| 828; 486399859; | 2160 | 2885 | 4130 | n/a | n/a |
| NZ_KB912942.1; 1655; | | | | | |
| 1656; 24/25 | | | | | |
| 829; 815864238; | n/a | 3623 | 4437 | n/a | n/a |
| NZ_LAJC01000053.1; | | | | | |
| 1657; 1658; 22/23 | | | | | |
| 830; 879201007; | 1380 | 2427 | 3820 | n/a | n/a |
| CKIK01000005.1; 1659; | | | | | |
| 1660; 19/20 | | | | | |
| 831; 655414006; | n/a | 3053 | n/a | n/a | 4225 |
| NZ_AUBE01000007.1; | | | | | |
| 1661; 1662; 57/58 | | | | | |
| 832; 749611130; | 2225 | 3331 | n/a | n/a | n/a |
| NZ_CDHL01000044.1; | | | | | |
| 1663; 1664; 22/23 | | | | | |
| 833; 664084661; | 1849 | 3535 | 4480 | n/a | n/a |
| NZ_JOED01000001.1; | | | | | |
| 1665; 1666; 33/34 | | | | | |
| 834; 256374160; | 1650 | 2778 | n/a | n/a | n/a |
| NC_013093.1; 1667; | | | | | |
| 1668; 40/41 | | | | | |
| 835; 822214995; | n/a | 3459 | n/a | n/a | n/a |
| NZ_CP007699.1; 1669; | | | | | |
| 1670; 73/74 | | | | | |
| 836; 664084661; | 1849 | 3533 | 4479 | n/a | n/a |
| NZ_JOED01000001.1; | | | | | |
| 1671; 1672; 33/34 | | | | | |
| 837; 357386972; | 1924 | 2746 | n/a | n/a | n/a |
| NC_016109.1; 1673; | | | | | |
| 1674; 26/27 | | | | | |
| 838; 822214995; | n/a | 2387 | n/a | n/a | n/a |
| NZ_CP007699.1; 1675; | | | | | |
| 1676; 73/74 | | | | | |
| 839; 558542923; | n/a | 3128 | n/a | n/a | 4150 |
| AWQW01000003.1; | | | | | |
| 1677; 1678; 19/20 | | | | | |
| 840; 671535174; | 1909 | 3390 | n/a | n/a | n/a |
| NZ_JOHY01000024.1; | | | | | |
| 1679; 1680; 29/30 | | | | | |
| 841; 671472153; | n/a | n/a | n/a | n/a | n/a |
| NZ_JOFR01000001.1; | | | | | |
| 1681; 1682; 21/22 | | | | | |
| 842; 919546534; | n/a | 3628 | n/a | n/a | n/a |
| NZ_JOEL01000027.1; | | | | | |
| 1683; 1684; 33/34 | | | | | |
| 843; 665530468; | n/a | 3581 | n/a | n/a | n/a |
| NZ_JOCD01000052.1; | | | | | |
| 1685; 1686; 26/27 | | | | | |
| 844; 563312125; | 1420 | 2475 | n/a | n/a | n/a |
| AYTZ01000052.1; | | | | | |
| 1687; 1688; 31/32 | | | | | |
| 845; 654993549; | n/a | 3265 | n/a | n/a | n/a |
| NZ_AZVE01000016.1; | | | | | |
| 1689; 1690; 29/30 | | | | | |
| 846; 663180071; | 1987 | 3081 | n/a | n/a | n/a |
| NZ_JOBE01000043.1; | | | | | |
| 1691; 1692; 28/29 | | | | | |
| 847; 664256887; | n/a | 3578 | n/a | n/a | 4499 |
| NZ_JODF01000036.1; | | | | | |
| 1693; 1694; 51/52 | | | | | |
| 848; 558542923; | n/a | 2473 | n/a | n/a | 3851 |
| AWQW01000003.1; | | | | | |
| 1695; 1696; 19/20 | | | | | |
| 849; 906344341; | 2247 | 3515 | 4472 | n/a | n/a |
| NZ_LFXA01000009.1; | | | | | |
| 1697; 1698; 25/26 | | | | | |
| 850; 563312125; | 1440 | 2502 | n/a | n/a | n/a |
| AYTZ01000052.1; | | | | | |
| 1699; 1700; 31/32 | | | | | |
| 851; 486330103; | 1724 | 2884 | n/a | n/a | n/a |
| NZ_KB913032.1; 1701; | | | | | |
| 1702; 31/32 | | | | | |
| 852; 663693444; | n/a | 3093 | n/a | n/a | n/a |
| NZ_JOFI01000027.1; | | | | | |
| 1703; 1704; 31/32 | | | | | |
| 853; 664299296; | 2198 | 3110 | 4282 | n/a | n/a |
| NZ_JOIK01000008.1; | | | | | |
| 1705; 1706; 25/26 | | | | | |
| 854; 925610911; | 1470 | 2542 | n/a | n/a | n/a |
| LGEE01000058.1; 1707; | | | | | |
| 1708; 28/29 | | | | | |
| 855; 663317502; | 2192 | 3085 | 4500 | n/a | n/a |
| NZ_JNZO01000008.1; | | | | | |
| 1709; 1710; 40/41 | | | | | |
| 856; 384145136; | n/a | 2714 | n/a | n/a | 4004 |
| NC_017186.1; 1711; | | | | | |
| 1712; 53/54 | | | | | |
| 857; 925610911; | 2259 | 3653 | n/a | n/a | n/a |
| LGEE01000058.1; 1713; | | | | | |
| 1714; 28/29 | | | | | |
| 858; 486324513; | 1715 | 2874 | n/a | n/a | n/a |
| NZ_KB913024.1; 1715; | | | | | |
| 1716; 37/38 | | | | | |
| 859; 759802587; | n/a | 3398 | n/a | n/a | 4512 |
| NZ_CP009438.1; 1717; | | | | | |
| 1718; 50/51 | | | | | |
| 860; 921220646; | 2069 | 3636 | n/a | n/a | n/a |
| NZ_JXYI02000059.1; | | | | | |
| 1719; 1720; 27/28 | | | | | |
| 861; 818476494; | n/a | 2391 | n/a | n/a | 3793 |
| KP274854.1; 1721; | | | | | |
| 1722; 53/54 | | | | | |
| 862; 365866490; | n/a | 3547 | n/a | n/a | n/a |
| NZ_AGSW01000226.1; | | | | | |
| 1723; 1724; 28/29 | | | | | |
| 863; 365866490; | n/a | 2446 | n/a | n/a | n/a |
| NZ_AGSW01000226.1; | | | | | |
| 1725; 1726; 28/29 | | | | | |
| 864; 937182893; | 2280 | 3688 | n/a | n/a | n/a |
| NZ_LFCW01000001.1; | | | | | |
| 1727; 1728; 31/32 | | | | | |
| 865; 484022237; | 1683 | 2831 | n/a | n/a | n/a |
| NZ_ANBD01000111.1; | | | | | |
| 1729; 1730; 22/23 | | | | | |
| 866; 747653426; | n/a | 2425 | n/a | n/a | 3818 |
| CDME01000011.1; | | | | | |
| 1731; 1732; 35/36 | | | | | |
| 867; 365866490; | n/a | 3569 | n/a | n/a | n/a |
| NZ_AGSW01000226.1; | | | | | |
| 1733; 1734; 28/29 | | | | | |
| 868; 926317398; | 2258 | 3652 | n/a | n/a | n/a |
| NZ_LGDO01000015.1; | | | | | |
| 1735; 1736; 27/28 | | | | | |
| 869; 746616581; | 1351 | 2383 | n/a | n/a | n/a |
| KF954512.1; 1737; | | | | | |
| 1738; 13/14 | | | | | |
| 870; 749658562; | 2019 | 3616 | n/a | n/a | n/a |
| NZ_CP010519.1; 1739; | | | | | |
| 1740; 29/30 | | | | | |
| 871; 487404592; | n/a | 2888 | n/a | n/a | 4132 |
| NZ_ARVW01000001.1; | | | | | |
| 1741; 1742; 41/42 | | | | | |
| 872; 389759651; | 1397 | 2449 | n/a | n/a | n/a |
| NZ_AJXS01000437.1; | | | | | |
| 1743; 1744; 26/27 | | | | | |
| 873; 930491003; | n/a | 3682 | n/a | n/a | 4542 |
| NZ_LJCU01000287.1; | | | | | |
| 1745; 1746; 29/30 | | | | | |
| 874; 484016556; | 1681 | 2986 | n/a | n/a | n/a |
| NZ_ANAX01000372.1; | | | | | |
| 1747; 1748; 27/28 | | | | | |
| 875; 433601838; | n/a | 3354 | n/a | n/a | 4045 |
| NC_019673.1; 1749; | | | | | |
| 1750; 44/45 | | | | | |
| 876; 483974021; | 1705 | 3270 | n/a | n/a | n/a |
| NZ_KB891893.1; 1751; | | | | | |
| 1752; 23/24 | | | | | |
| 877; 930491003; | n/a | 2545 | n/a | n/a | 3887 |
| NZ_LJCU01000287.1; | | | | | |
| 1753; 1754; 29/30 | | | | | |
| 878; 749658562; | 1352 | 2384 | n/a | n/a | n/a |
| NZ_CP010519.1; 1755; | | | | | |
| 1756; 29/30 | | | | | |
| 879; 759755931; | 2188 | 3396 | n/a | n/a | n/a |
| NZ_JAIY01000003.1; | | | | | |
| 1757; 1758; 27/28 | | | | | |
| 880; 484007204; | 1678 | 2821 | 4086 | n/a | n/a |
| NZ_ANAC01000034.1; | | | | | |
| 1759; 1760; 25/26 | | | | | |
| 881; 433601838; | n/a | 2416 | n/a | n/a | 3811 |
| NC_019673.1; 1761; | | | | | |
| 1762; 44/45 | | | | | |
| 882; 254387191; | 1554 | 3542 | n/a | n/a | n/a |
| NZ_DS570483.1; 1763; | | | | | |
| 1764; 27/28 | | | | | |
| 883; 345007457; | 1623 | 2740 | 4024 | n/a | n/a |
| NC_015951.1; 1765; | | | | | |
| 1766; 38/39 | | | | | |
| 884; 297558985; | 2138 | 2713 | n/a | n/a | n/a |
| NC_014210.1; 1767; | | | | | |
| 1768; 27/28 | | | | | |
| 885; 927872504; | 2270 | 3457 | 4439 | n/a | n/a |
| NZ_CP011452.2; 1769; | | | | | |
| 1770; 12/13 | | | | | |
| 886; 970555001; | 2334 | 3759 | 4593 | n/a | n/a |
| NZ_LNRZ01000006.1; | | | | | |
| 1771; 1772; 25/26 | | | | | |
| 887; 960424655; | 2331 | 3754 | 4589 | n/a | n/a |
| NZ_CYUE01000025.1; | | | | | |
| 1773; 1774; 21/22 | | | | | |
| 888; 483994857; | 1723 | 2989 | 4129 | n/a | n/a |
| NZ_KB893599.1; 1775; | | | | | |
| 1776; 33/34 | | | | | |
| 889; 817524426; | 2093 | 3452 | 4435 | n/a | n/a |
| NZ_CP010429.1; 1777; | | | | | |
| 1778; 33/34 | | | | | |
| 890; 970361514; | 1481 | 2556 | 3896 | n/a | n/a |
| LOCL01000028.1; 1779; | | | | | |
| 1780; 21/22 | | | | | |
| 891; 970574347; | 2335 | 3760 | 4008 | n/a | n/a |
| NZ_LNZF01000001.1; | | | | | |
| 1781; 1782; 20/21 | | | | | |
| 892; 970574347; | 1610 | 3758 | 4373 | n/a | n/a |
| NZ_LNZF01000001.1; | | | | | |
| 1783; 1784; 20/21 | | | | | |
| 893; 961447255; | 1365 | 2402 | 3799 | n/a | n/a |
| CP013653.1; 1785; | | | | | |
| 1786; 20/21 | | | | | |
| 894; 283814236; | 1329 | 2354 | 3766 | n/a | n/a |
| CP001769.1; 1787; | | | | | |
| 1788; 35/36 | | | | | |
| 895; 746187486; | n/a | 3304 | 4506 | n/a | n/a |
| NZ_MSY01000011.1; | | | | | |
| 1789; 1790; 12/13 | | | | | |
| 896; 960412751; | 2330 | 3753 | 4588 | n/a | n/a |
| NZ_LN881722.1; 1791; | | | | | |
| 1792; 19/20 | | | | | |
| 897; 970293907; | n/a | 2555 | n/a | n/a | n/a |
| LOHP01000076.1; 1793; | | | | | |
| 1794; 22/23 | | | | | |
| 898; 943388237; | 2295 | 3704 | 4547 | n/a | n/a |
| NZ_LIQD01000001.1; | | | | | |
| 1795; 1796; 21/22 | | | | | |
| 899; 944415035; | n/a | 3719 | n/a | n/a | 4562 |
| NZ_LIRG01000370.1; | | | | | |
| 1797; 1798; 51/52 | | | | | |
| 900; 944005810; | 2304 | 3714 | 4557 | n/a | n/a |
| NZ_LIQT01000057.1; | | | | | |
| 1799; 1800; 28/29 | | | | | |
| 901; 944020089; | n/a | 3716 | n/a | n/a | 4559 |
| NZ_LIPR01000230.1; | | | | | |
| 1801; 1802; 51/52 | | | | | |
| 902; 944020089; | n/a | 3718 | n/a | n/a | 4561 |
| NZ_LIPR01000230.1; | | | | | |
| 1803; 1804; 51/52 | | | | | |
| 903; 943922567; | n/a | 3711 | 4554 | n/a | n/a |
| NZ_LIQU01000247.1; | | | | | |
| 1805; 1806; 29/30 | | | | | |
| 904; 969919061; | 2333 | 3756 | 4591 | n/a | n/a |
| NZ_LDRR01000065.1; | | | | | |
| 1807; 1808; 21/22 | | | | | |
| 905; 969919061; | 2333 | 3756 | 4591 | n/a | n/a |
| NZ_LDRR01000065.1; | | | | | |
| 1809; 1810; 21/22 | | | | | |
| 906; 969919061; | 2333 | 3757 | 4592 | n/a | n/a |
| NZ_LDRR01000065.1; | | | | | |
| 1811; 1812; 21/22 | | | | | |
| 907; 969919061; | 2333 | 3757 | 4592 | n/a | n/a |
| NZ_LDRR01000065.1; | | | | | |
| 1813; 1814; 21/22 | | | | | |
| 908; 969919061; | 2332 | 3755 | 4590 | n/a | n/a |
| NZ_LDRR01000065.1; | | | | | |
| 1815; 1816; 21/22 | | | | | |
| 909; 969919061; | 2332 | 3755 | 4590 | n/a | n/a |
| NZ_LDRR01000065.1; | | | | | |
| 1817; 1818; 21/22 | | | | | |
| 910; 483454700; | 1722 | 2987 | 4128 | n/a | n/a |
| NZ_KB903974.1; 1819; | | | | | |
| 1820; 31/32 | | | | | |
| 911; 970579907; | 2336 | 3761 | n/a | n/a | n/a |
| NZ_KQ759763.1; 1821; | | | | | |
| 1822; 27/28 | | | | | |
| 912; 947401208; | 2311 | 3725 | n/a | n/a | n/a |
| NZ_LMKW01000010.1; | | | | | |
| 1823; 1824; 20/21 | | | | | |
| 913; 941965142; | 2293 | 3702 | n/a | n/a | n/a |
| NZ_LKIT01000002.1; | | | | | |
| 1825; 1826; 26/27 | | | | | |
| 914; 941965142; | 2293 | 3702 | n/a | n/a | n/a |
| NZ_LKIT01000002.1; | | | | | |
| 1827; 1828; 29/30 | | | | | |
| 915; 312193897; | n/a | 2720 | n/a | n/a | n/a |
| NC_014666.1; 1829; | | | | | |
| 1830; 35/36 | | | | | |
| 916; 736762362; | 1939 | 3187 | 4323 | n/a | n/a |
| NZ_CCDN010000009.1; | | | | | |
| 1831; 1832; 19/20 | | | | | |
| 917; 651596980; | 1784 | 2997 | 4190 | n/a | n/a |
| NZ_AXVB01000011.1; | | | | | |
| 1833; 1834; 19/20 | | | | | |
| 918; 850356871; | 2110 | 3482 | 4454 | n/a | n/a |
| NZ_LDWN01000016.1; | | | | | |
| 1835; 1836; 11/12 | | | | | |
| 919; 924654439; | 2253 | 3644 | 4523 | n/a | n/a |
| NZ_LIUS01000003.1; | | | | | |
| 1837; 1838; 19/20 | | | | | |
| 920; 238801497; | 1706 | 2620 | 3897 | n/a | n/a |
| NZ_CM000745.1; 1839; | | | | | |
| 1840; 19/20 | | | | | |
| 921; 651983111; | 2171 | 3001 | 4192 | n/a | n/a |
| NZ_KE387239.1; 1841; | | | | | |
| 1842; 23124 | | | | | |
| 922; 727343482; | 1706 | 2593 | 3897 | n/a | n/a |
| NZ_JMQD01000030.1; | | | | | |
| 1843; 1844; 19/20 | | | | | |
| 923; 423557538; | 1499 | 2580 | 3913 | n/a | n/a |
| NZ_JH792114.1; 1845; | | | | | |
| 1846; 19/20 | | | | | |
| 924; 727343482; | 1706 | 3175 | 3897 | n/a | n/a |
| NZ_JMQD01000030.1; | | | | | |
| 1847; 1848; 19/20 | | | | | |
| 925; 727343482; | 1486 | 2789 | 4066 | n/a | n/a |
| NZ_JMQD01000030.1; | | | | | |
| 1849; 1850; 19/20 | | | | | |
| 926; 727343482; | 1486 | 2785 | 4066 | n/a | n/a |
| NZ_JMQD01000030.1; | | | | | |
| 1851; 1852; 19/20 | | | | | |
| 927; 727343482; | 1486 | 2786 | 4067 | n/a | n/a |
| NZ_JMQD01000030.1; | | | | | |
| 1853; 1854; 19/20 | | | | | |
| 928; 727343482; | 1762 | 2961 | 3897 | n/a | n/a |
| NZ_JMQD01000030.1; | | | | | |
| 1855; 1856; 19/20 | | | | | |
| 929; 487368297; | 1718 | 2877 | 4122 | n/a | n/a |
| NZ_KB910953.1; 1857; | | | | | |
| 1858; 19/20 | | | | | |
| 930; 423614674; | 1488 | 2562 | 3904 | n/a | n/a |
| NZ_JH792165.1; 1859; | | | | | |
| 1860; 19/20 | | | | | |
| 931; 727343482; | 1502 | 2584 | 3916 | n/a | n/a |
| NZ_JMQD01000030.1; | | | | | |
| 1861; 1862; 19/20 | | | | | |
| 932; 727343482; | 1486 | 2788 | 4066 | n/a | n/a |
| NZ_JMQD01000030.1; | | | | | |
| 1863; 1864; 19/20 | | | | | |
| 933; 727343482; | 1486 | 2583 | 3897 | n/a | n/a |
| NZ_JMQD01000030.1; | | | | | |
| 1865; 1866; 19/20 | | | | | |
| 934; 736214556; | 1935 | 3183 | 4321 | n/a | n/a |
| NZ_KN360955.1; 1867; | | | | | |
| 1868; 19/20 | | | | | |
| 935; 507060152; | 1653 | 2787 | 4068 | n/a | n/a |
| NZ_KB976714.1; 1869; | | | | | |
| 1870; 19/20 | | | | | |
| 936; 727343482; | 1486 | 2570 | 3897 | n/a | n/a |
| NZ_JMQD01000030.1; | | | | | |
| 1871; 1872; 19/20 | | | | | |
| 937; 737456981; | 1948 | 3201 | 4502 | n/a | n/a |
| NZ_KN050811.1; 1873; | | | | | |
| 1874; 11/12 | | | | | |
| 938; 880954155; | 2118 | 3491 | 4462 | n/a | n/a |
| NZ_JVPL01000109.1; | | | | | |
| 1875; 1876; 19/20 | | | | | |
| 939; 751619763; | 2026 | 3348 | 4385 | n/a | n/a |
| NZ_JXRP01000009.1; | | | | | |
| 1877; 1878; 13/14 | | | | | |
| 940; 727343482; | 1486 | 3384 | 3897 | n/a | n/a |
| NZ_JMQD01000030.1; | | | | | |
| 1879; 1880; 19/20 | | | | | |
| 941; 806951735; | 1490 | 2561 | 3905 | n/a | n/a |
| NZ_JSFD01000011.1; | | | | | |
| 1881; 1882; 19/20 | | | | | |
| 942; 736160933; | 1934 | 3182 | 4320 | n/a | n/a |
| NZ_JQMI01000015.1; | | | | | |
| 1883; 1884; 19/20 | | | | | |
| 943; 736160933; | 1934 | 3182 | 4320 | n/a | n/a |
| NZ_JQMI01000015.1; | | | | | |
| 1885; 1886; 19/20 | | | | | |
| 944; 872696015; | 2115 | 3485 | 4460 | n/a | n/a |
| NZ_LABO01000035.1; | | | | | |
| 1887; 1888; 19/20 | | | | | |
| 945; 806951735; | 1493 | 2572 | 3905 | n/a | n/a |
| NZ_JSFD01000011.1; | | | | | |
| 1889; 1890; 19/20 | | | | | |
| 946; 806951735; | 2087 | 3444 | 3905 | n/a | n/a |
| NZ_JSFD01000011.1; | | | | | |
| 1891; 1892; 19/20 | | | | | |
| 947; 950170460; | 2323 | 3742 | 4580 | n/a | n/a |
| NZ_LMTA01000046.1; | | | | | |
| 1893; 1894; 19/20 | | | | | |
| 948; 872696015; | 1498 | 2585 | 3917 | n/a | n/a |
| NZ_LABO01000035.1; | | | | | |
| 1895; 1896; 19/20 | | | | | |
| 949; 163938013; | 1596 | 2695 | 3991 | n/a | n/a |
| NC_010184.1; 1897; | | | | | |
| 1898; 13/14 | | | | | |
| 950; 872696015; | 1498 | 2782 | 4064 | n/a | n/a |
| NZ_LABO01000035.1; | | | | | |
| 1899; 1900; 19/20 | | | | | |
| 951; 238801491; | 1487 | 2560 | 3902 | n/a | n/a |
| NZ_CM000739.1; 1901; | | | | | |
| 1902; 19/20 | | | | | |
| 952; 657629081; | | 1837 | 3068 | 4237 | n/a |
| NZ_AYPV01000024.1; | | | | | |
| 1903; 1904; 19/20 | | | | | |
| 953; 507035131; | 1652 | 2783 | 4065 | n/a | n/a |
| NZ_KB976800.1; 1905; | | | | | |
| 1906; 19/20 | | | | | |
| 954; 737576092; | 1951 | 3205 | 4331 | n/a | n/a |
| NZ_JRNX01000441.1; | | | | | |
| 1907; 1908; 3/4 | | | | | |
| 955; 947983982; | 2321 | 3737 | 4578 | n/a | n/a |
| NZ_LMRV01000044.1; | | | | | |
| 1909; 1910; 11/12 | | | | | |
| 956; 946400391; | 2324 | 3743 | 4581 | n/a | n/a |
| LMRY01000003.1; | | | | | |
| 1911; 1912; 23/24 | | | | | |
| 957; 423456860; | 1495 | 2568 | 3906 | n/a | n/a |
| NZ_JH791975.1; 1913; | | | | | |
| 1914; 19/20 | | | | | |
| 958; 514340871; | 1494 | 2575 | 3908 | n/a | n/a |
| NZ_KE150045.1; 1915; | | | | | |
| 1916; 19/20 | | | | | |
| 959; 946400391; | 1480 | 2554 | 3895 | n/a | n/a |
| LMRY01000003.1; | | | | | |
| 1917; 1918; 23/24 | | | | | |
| 960; 655103160; | 1825 | 3046 | 4220 | n/a | n/a |
| NZ_JMLS01000021.1; | | | | | |
| 1919; 1920; 11/12 | | | | | |
| 961; 910095435; | 1930 | 2577 | 3910 | n/a | n/a |
| NZ_JNLY01000005.1; | | | | | |
| 1921; 1922; 19/20 | | | | | |
| 962; 910095435; | 1931 | 2581 | 3910 | n/a | n/a |
| NZ_JNLY01000005.1; | | | | | |
| 1923; 1924; 19/20 | | | | | |
| 963; 910095435; | 1931 | 3519 | 4474 | n/a | n/a |
| NZ_JNLY01000005.1; | | | | | |
| 1925; 1926; 19/20 | | | | | |
| 964; 910095435; | 1930 | 3174 | 3910 | n/a | n/a |
| NZ_JNLY01000005.1; | | | | | |
| 1927; 1928; 19/20 | | | | | |
| 965; 922780240; | 2248 | 3638 | 4521 | n/a | n/a |
| NZ_LIGH01000001.1; | | | | | |
| 1929; 1930; 21/22 | | | | | |
| 966; 929005248; | 2275 | 3676 | 4539 | n/a | n/a |
| NZ_LGHP01000003.1; | | | | | |
| 1931; 1932; 21/22 | | | | | |
| 967; 767005659; | n/a | 3428 | n/a | n/a | n/a |
| NZ_CP010976.1; 1933; | | | | | |
| 1934; 19/20 | | | | | |
| 968; 507017505; | 1651 | 2780 | 4063 | n/a | n/a |
| NZ_KB976530.1; 1935; | | | | | |
| 1936; 19/20 | | | | | |
| 969; 423520617; | 1498 | 2579 | 3912 | n/a | n/a |
| NZ_JH792148.1; 1937; | | | | | |
| 1938; 19/20 | | | | | |
| 970; 910095435; | 1930 | 2574 | 4317 | n/a | n/a |
| NZ_JNLY01000005.1; | | | | | |
| 1939; 1940; 19/20 | | | | | |
| 971; 507020427; | 1497 | 2578 | 3911 | n/a | n/a |
| NZ_KB976152.1; 1941; | | | | | |
| 1942; 19/20 | | | | | |
| 972; 910095435; | 1488 | 2565 | 3900 | n/a | n/a |
| NZ_JNLY01000005.1; | | | | | |
| 1943; 1944; 19/20 | | | | | |
| 973; 483299154; | 1672 | 2813 | 4083 | n/a | n/a |
| NZ_AMGD01000001.1; | | | | | |
| 1945; 1946; 19/20 | | | | | |
| 974; 483299154; | 1672 | 2813 | 4083 | n/a | n/a |
| NZ_AMGD01000001.1; | | | | | |
| 1947; 1948; 19/20 | | | | | |
| 975; 910095435; | 1488 | 2784 | 3900 | n/a | n/a |
| NZ_JNLY01000005.1; | | | | | |
| 1949; 1950; 19/20 | | | | | |
| 976; 423468694; | 1496 | 2576 | 3909 | n/a | n/a |
| NZ_JH804628.1; 1951; | | | | | |
| 1952; 19/20 | | | | | |
| 977; 507020427; | 1491 | 2569 | 3898 | n/a | n/a |
| NZ_KB976152.1; 1953; | | | | | |
| 1954; 19/20 | | | | | |
| 978; 910095435; | 1488 | 2564 | 3900 | n/a | n/a |
| NZ_JNLY01000005.1; | | | | | |
| 1955; 1956; 19/20 | | | | | |
| 979; 910095435; | 1488 | 2566 | 3900 | n/a | n/a |
| NZ_JNLY01000005.1; | | | | | |
| 1957; 1958; 19/20 | | | | | |
| 980; 423609285; | 1501 | 2582 | 3915 | n/a | n/a |
| NZ_JH792232.1; 1959; | | | | | |
| 1960; 19/20 | | | | | |
| 981; 947966412; | 2320 | 3736 | 4576 | n/a | n/a |
| NZ_LMSD01000001.1; | | | | | |
| 1961; 1962; 19/20 | | | | | |
| 982; 947966412; | 2320 | 3736 | 4576 | n/a | n/a |
| NZ_LMSD01000001.1; | | | | | |
| 1963; 1964; 19/20 | | | | | |
| 983; 507020427; | 1497 | 2781 | 3911 | n/a | n/a |
| NZ_KB976152.1; 1965; | | | | | |
| 1966; 19/20 | | | | | |
| 984; 910095435; | 1489 | 2567 | 3899 | n/a | n/a |
| NZ_JNLY01000005.1; | | | | | |
| 1967; 1968; 19/20 | | | | | |
| 985; 950280827; | 2325 | 3744 | 4583 | n/a | n/a |
| NZ_LMSJ01000026.1; | | | | | |
| 1969; 1970; 19/20 | | | | | |
| 986; 656249802; | 1833 | 3062 | 4230 | n/a | n/a |
| NZ_AUGY01000047.1; | | | | | |
| 1971; 1972; 19/20 | | | | | |
| 987; 238801471; | 1500 | 2573 | 3914 | n/a | n/a |
| NZ_CM000719.1; 1973; | | | | | |
| 1974; 19/20 | | | | | |
| 988; 485048843; | 1711 | 2867 | 4111 | n/a | n/a |
| NZ_ALEG01000067.1; | | | | | |
| 1975; 1976; 19/20 | | | | | |
| 989; 647636934; | 1773 | 2979 | 4182 | n/a | n/a |
| NZ_JANV01000106.1; | | | | | |
| 1977; 1978; 19/20 | | | | | |
| 990; 910095435; | 1488 | 2563 | 3901 | n/a | n/a |
| NZ_JNLY01000005.1; | | | | | |
| 1979; 1980; 19/20 | | | | | |
| 991; 817541164; | 2092 | 3454 | 4438 | n/a | n/a |
| NZ_LATZ01000026.1; | | | | | |
| 1981; 1982; 19/20 | | | | | |
| 992; 488570484; | 2032 | 2770 | 4057 | n/a | n/a |
| NC_021171.1; 1983; | | | | | |
| 1984; 19/20 | | | | | |
| 993; 914730676; | 2149 | 3540 | 4481 | n/a | n/a |
| NZ_LFQJ01000032.1; | | | | | |
| 1985; 1986; 19/20 | | | | | |
| 994; 928874573; | 2052 | 3670 | 4404 | n/a | n/a |
| NZ_LIXL01000208.1; | | | | | |
| 1987; 1988; 19/20 | | | | | |
| 995; 928874573; | 2052 | 3670 | 4404 | n/a | n/a |
| NZ_LIXL01000208.1; | | | | | |
| 1989; 1990; 19/20 | | | | | |
| 996; 655165706; | 1969 | 3050 | 4222 | n/a | n/a |
| NZ_KE383843.1; 1991; | | | | | |
| 1992; 11/12 | | | | | |
| 997; 656245934; | 1832 | 3060 | 4229 | n/a | n/a |
| NZ_KE383845.1; 1993; | | | | | |
| 1994; 19/20 | | | | | |
| 998; 928874573; | 2052 | 3385 | 4404 | n/a | n/a |
| NZ_LIXL01000208.1; | | | | | |
| 1995; 1996; 19/20 | | | | | |
| 999; 928874573; | 2052 | 3385 | 4404 | n/a | n/a |
| NZ_LIXL01000208.1; | | | | | |
| 1997; 1998; 19/20 | | | | | |
| 1000; 924371245; | n/a | 3642 | n/a | n/a | n/a |
| NZ_LITP01000001.1; | | | | | |
| 1999; 2000; 19/20 | | | | | |
| 1001; 654948246; | 1819 | 3040 | 4216 | n/a | n/a |
| NZ_KI632505.1; 2001; | | | | | |
| 2002; 11/12 | | | | | |
| 1002; 657210762; | 2051 | 2750 | 4033 | n/a | n/a |
| NZ_AXZS01000018.1; | | | | | |
| 2003; 2004; 19/20 | | | | | |
| 1003; 571146044; | 1747 | 2916 | 4153 | n/a | n/a |
| BAUW01000006.1; | | | | | |
| 2005; 2006; 19/20 | | | | | |
| 1004; 935460965; | n/a | 3685 | n/a | n/a | n/a |
| NZ_LIUT01000006.1; | | | | | |
| 2007; 2008; 19/20 | | | | | |
| 1005; 651516582; | 2175 | 2995 | 4189 | n/a | n/a |
| NZ_JAEK01000001.1; | | | | | |
| 2009; 2010; 19/20 | | | | | |
| 1006; 657210762; | 1820 | 3042 | 4217 | n/a | n/a |
| NZ_AXZS01000018.1; | | | | | |
| 2011; 2012; 19/20 | | | | | |
| 1007; 657210762; | 2105 | 3476 | 4448 | n/a | n/a |
| NZ_AXZS01000018.1; | | | | | |
| 2013; 2014; 19/20 | | | | | |
| 1008; 723602665; | 1929 | 3173 | 4315 | n/a | n/a |
| NZ_JPIE01000001.1; | | | | | |
| 2015; 2016; 19/20 | | | | | |
| 1009; 657210762; | 1834 | 3065 | 4233 | n/a | n/a |
| NZ_AXZS01000018.1; | | | | | |
| 2017; 2018; 19/20 | | | | | |
| 1010; 933903534; | 1475 | 2549 | 3891 | n/a | n/a |
| LIXZ01000017.1; 2019; | | | | | |
| 2020; 11/12 | | | | | |
| 1011; 654954291; | n/a | 3041 | n/a | n/a | n/a |
| NZ_JAEO01000006.1; | | | | | |
| 2021; 2022; 19/20 | | | | | |
| 1012; 238801472; | 1482 | 2559 | 4316 | n/a | n/a |
| NZ_CM000720.1; 2023; | | | | | |
| 2024; 11/12 | | | | | |
| 1013; 651516582; | 2175 | 2995 | 4189 | n/a | n/a |
| NZ_JAEK01000001.1; | | | | | |
| 2025; 2026; 19/20 | | | | | |
| 1014; 910095435; | 1340 | 2369 | 3776 | n/a | n/a |
| NZ_JNLY01000005.1; | | | | | |
| 2027; 2028; 19/20 | | | | | |
| 1015; 403048279; | n/a | 2671 | n/a | n/a | n/a |
| NZ_HE610988.1; 2029; | | | | | |
| 2030; 19/20 | | | | | |
| 1016; 750677319; | 2222 | 3339 | 4509 | n/a | n/a |
| NZ_CBQR020000171.1; | | | | | |
| 2031; 2032; 20/21 | | | | | |
| 1017; 849078078; | 2109 | 3481 | 4453 | n/a | n/a |
| NZ_LFJO01000006.1; | | | | | |
| 2033; 2034; 18/19 | | | | | |
| 1018; 890672806; | 1712 | 3329 | 4112 | n/a | n/a |
| NZ_CP011974.1; 2035; | | | | | |
| 2036; 0/1 | | | | | |
| 1019; 890672806; | 1712 | 3446 | 4112 | n/a | n/a |
| NZ_CP011974.1; 2037; | | | | | |
| 2038; 0/1 | | | | | |
| 1020; 727078508; | n/a | 2514 | n/a | n/a | n/a |
| JRNV01000046.1; 2039; | | | | | |
| 2040; 19/20 | | | | | |
| 1021; 749299172; | 1995 | 3278 | 4363 | n/a | n/a |
| NZ_CP009241.1; 2041; | | | | | |
| 2042; 19/20 | | | | | |
| 1022; 652787974; | 2169 | 3015 | 4203 | n/a | n/a |
| NZ_AUCP01000055.1; | | | | | |
| 2043; 2044; 50/51 | | | | | |
| 1023; 652787974; | 2169 | 3015 | 4203 | n/a | n/a |
| NZ_AUCP01000055.1; | | | | | |
| 2045; 2046; 23/24 | | | | | |
| 1024; 486346141; | 1717 | 2876 | 4121 | n/a | n/a |
| NZ_KB910518.1; 2047; | | | | | |
| 2048; 19/20 | | | | | |
| 1025; 951610263; | 2328 | 3747 | 4586 | n/a | n/a |
| NZ_LMBV01000004.1; | | | | | |
| 2049; 2050; 19/20 | | | | | |
| 1026; 354585485; | n/a | 2629 | n/a | n/a | n/a |
| NZ_AGIP01000020.1; | | | | | |
| 2051; 2052; 19/20 | | | | | |
| 1027; 940346731; | 2292 | 3701 | 4546 | n/a | n/a |
| NZ_LJCO01000107.1; | | | | | |
| 2053; 2054; 19/20 | | | | | |
| 1028; 880997761; | 2119 | 3492 | 4463 | n/a | n/a |
| NZ_JVDT01000118.1; | | | | | |
| 2055; 2056; 20/21 | | | | | |
| 1029; 880997761; | 1910 | 3132 | 4300 | n/a | n/a |
| NZ_JVDT01000118.1; | | | | | |
| 2057; 2058; 20/21 | | | | | |
| 1030; 746258261; | 2038 | 3369 | 4514 | n/a | n/a |
| NZ_JUEI01000069.1; | | | | | |
| 2059; 2060; 19/20 | | | | | |
| 1031; 849059098; | 2108 | 3480 | 4452 | n/a | n/a |
| NZ_LDUE01000022.1; | | | | | |
| 2061; 2062; 22/23 | | | | | |
| 1032; 746258261; | 2003 | 3309 | 4367 | n/a | n/a |
| NZ_JUEI01000069.1; | | | | | |
| 2063; 2064; 19/20 | | | | | |
| 1033; 754884871; | 2038 | 3375 | 4513 | n/a | n/a |
| NZ_CP009282.1; 2065; | | | | | |
| 2066; 19/20 | | | | | |
| 1034; 939708105; | 2291 | 3700 | 4545 | n/a | n/a |
| NZ_LN831205.1; 2067; | | | | | |
| 2068; 19/20 | | | | | |
| 1035; 738803633; | 1970 | 3225 | 4341 | n/a | n/a |
| NZ_ASPS01000022.1; | | | | | |
| 2069; 2070; 19/20 | | | | | |
| 1036; 754841195; | 2044 | 3374 | 4398 | n/a | n/a |
| NZ_CCDG010000069.1; | | | | | |
| 2071; 2072; 19/20 | | | | | |
| 1037; 754841195; | 2016 | 3326 | 4372 | n/a | n/a |
| NZ_CCDG010000069.1; | | | | | |
| 2073; 2074; 19/20 | | | | | |
| 1038; 751586078; | 2227 | 3346 | 4384 | n/a | n/a |
| NZ_JXRR01000001.1; | | | | | |
| 2075; 2076; 19/20 | | | | | |
| 1039; 970574347; | n/a | 2749 | 4032 | n/a | n/a |
| NZ_LNZF01000001.1; | | | | | |
| 2077; 2078; 20/21 | | | | | |
| 1040; 754841195; | 2041 | 3372 | 4395 | n/a | n/a |
| NZ_CCDG010000069.1; | | | | | |
| 2079; 2080; 19/20 | | | | | |
| 1041; 927084730; | 2267 | 3664 | 4534 | n/a | n/a |
| NZ_LITU01000050.1; | | | | | |
| 2081; 2082; 20/21 | | | | | |
| 1042; 738716739; | 1965 | 3220 | 4339 | n/a | n/a |
| NZ_ASPU01000015.1; | | | | | |
| 2083; 2084; 20/21 | | | | | |
| 1043; 738716739; | 1965 | 3220 | 4339 | n/a | n/a |
| NZ_ASPU01000015.1; | | | | | |
| 2085; 2086; 20/21 | | | | | |
| 1044; 639451286; | 1756 | 2956 | 4169 | n/a | n/a |
| NZ_AWUK01000007.1; | | | | | |
| 2087; 2088; 20/21 | | | | | |
| 1045; 738803633; | 1967 | 3223 | 4340 | n/a | n/a |
| NZ_ASPS01000022.1; | | | | | |
| 2089; 2090; 19/20 | | | | | |
| 1046; 484070054; | 1688 | 2838 | 4097 | n/a | n/a |
| NZ_ANHX01000029.1; | | | | | |
| 2091; 2092; 20/21 | | | | | |
| 1047; 484070054; | 1688 | 2838 | 4097 | n/a | n/a |
| NZ_ANHX01000029.1; | | | | | |
| 2093; 2094; 20/21 | | | | | |
| 1048; 754841195; | 2043 | 3373 | 4397 | n/a | n/a |
| NZ_CCDG010000069.1; | | | | | |
| 2095; 2096; 19/20 | | | | | |
| 1049; 948045460; | 2322 | 3739 | 4579 | n/a | n/a |
| NZ_LMFO01000023.1; | | | | | |
| 2097; 2098; 22/23 | | | | | |
| 1050; 652787974; | 2169 | 3016 | 4203 | n/a | n/a |
| NZ_AUCP01000055.1; | | | | | |
| 2099; 2100; 50/51 | | | | | |
| 1051; 652787974; | 2169 | 3016 | 4203 | n/a | n/a |
| NZ_AUCP01000055.1; | | | | | |
| 2101; 2102; 23/24 | | | | | |
| 1052; 924434005; | 1459 | 2530 | 3875 | n/a | n/a |
| LIYK01000027.1; 2103; | | | | | |
| 2104; 20/21 | | | | | |
| 1053; 926268043; | 2257 | 3648 | 4524 | n/a | n/a |
| NZ_CP012600.1; 2105; | | | | | |
| 2106; 19/20 | | | | | |
| 1054; 374605177; | 2023 | 2626 | 3940 | n/a | n/a |
| NZ_AHKH01000064.1; | | | | | |
| 2107; 2108; 19/20 | | | | | |
| 1055; 392955666; | 1541 | 2630 | 3943 | n/a | n/a |
| NZ_AKKV01000020.1; | | | | | |
| 2109; 2110; 19/20 | | | | | |
| 1056; 651937013; | 1786 | 2999 | 4191 | n/a | n/a |
| NZ_JHYI01000013.1; | | | | | |
| 2111; 2112; 19/20 | | | | | |
| 1057; 843088522; | 2106 | 3478 | 4449 | n/a | n/a |
| NZ_BBIW01000001.1; | | | | | |
| 2113; 2114; 17/18 | | | | | |
| 1058; 656245934; | 1832 | 3060 | 4229 | n/a | n/a |
| NZ_KE383845.1; 2115; | | | | | |
| 2116; 19/20 | | | | | |
| 1059; 651937013; | 1786 | 2999 | 4191 | n/a | n/a |
| NZ_JHYI01000013.1; | | | | | |
| 2117; 2118; 19/20 | | | | | |
| 1060; 430748349; | 1640 | 2767 | 4055 | n/a | n/a |
| NC_019897.1; 2119; | | | | | |
| 2120; 20/21 | | | | | |
| 1061; 947983982; | 2321 | 3737 | 4578 | n/a | n/a |
| NZ_LMRV01000044.1; | | | | | |
| 2121; 2122; 11/12 | | | | | |
| 1062; 749182744; | 2015 | 3596 | 4371 | n/a | n/a |
| NZ_CP009416.1; 2123; | | | | | |
| 2124; 19/20 | | | | | |
| 1063; 802929558; | 2235 | 3059 | 4228 | n/a | n/a |
| NZ_CP009933.1; 2125; | | | | | |
| 2126; 20/21 | | | | | |
| 1064; 550916528; | 1733 | 2898 | 4138 | n/a | n/a |
| NC_022571.1; 2127; | | | | | |
| 2128; 25/26 | | | | | |
| 1065; 950938054; | 2326 | 3745 | 3907 | n/a | n/a |
| NZ_CIHL01000007.1; | | | | | |
| 2129; 2130; 19/20 | | | | | |
| 1066; 571146044; | 1431 | 2490 | 3859 | n/a | n/a |
| BAUW01000006.1; | | | | | |
| 2131; 2132; 19/20 | | | | | |
| 1067; 571146044; | 1431 | 2490 | 3859 | n/a | n/a |
| BAUW01000006.1; | | | | | |
| 2133; 2134; 19/20 | | | | | |
| 1068; 427733619; | 2221 | 2760 | 4048 | n/a | n/a |
| NC_019678.1; 2135; | | | | | |
| 2136; 22/23 | | | | | |
| 1069; 657706549; | 1838 | 3070 | n/a | n/a | n/a |
| NZ_JNLM01000001.1; | | | | | |
| 2137; 2138; 44/45 | | | | | |
| 1070; 514429123; | 1654 | 2791 | 4484 | n/a | n/a |
| NZ_KE332377.1; 2139; | | | | | |
| 2140; 29/30 | | | | | |
| 1071; 514429123; | 1654 | 2791 | 4484 | n/a | n/a |
| NZ_KE332377.1; 2141; | | | | | |
| 2142; 29/30 | | | | | |
| 1072; 514429123; | 1654 | 2791 | 4484 | n/a | n/a |
| NZ_KE332377.1; 2143; | | | | | |
| 2144; 29/30 | | | | | |
| 1073; 931536013; | 1474 | 2548 | 3890 | n/a | n/a |
| LJUL01000022.1; 2145; | | | | | |
| 2146; 38/39 | | | | | |
| 1074; 931536013; | 1474 | 2548 | 3890 | n/a | n/a |
| LJUL01000022.1; 2147; | | | | | |
| 2148; 38/39 | | | | | |
| 1075; 931536013; | 1474 | 2548 | 3890 | n/a | n/a |
| LJUL01000022.1; 2149; | | | | | |
| 2150; 38/39 | | | | | |
| 1076; 931536013; | 1474 | 2548 | 3890 | n/a | n/a |
| LJUL01000022.1; 2151; | | | | | |
| 2152; 38/39 | | | | | |
| 1077; 931536013; | 1474 | 2548 | 3890 | n/a | n/a |
| LJUL01000022.1; 2153; | | | | | |
| 2154; 38/39 | | | | | |
| 1078; 931536013; | 1474 | 2548 | 3890 | n/a | n/a |
| LJUL01000022.1; 2155; | | | | | |
| 2156; 38/39 | | | | | |
| 1079; 575082509; | 1432 | 2492 | 3860 | n/a | n/a |
| BAVS01000030.1; | | | | | |
| 2157; 2158; 19/20 | | | | | |
| 1080; 930349143; | 1362 | 2398 | 3798 | n/a | n/a |
| CP012036.1; 2159; | | | | | |
| 2160; 21/22 | | | | | |
| 1081; 575082509; | 1432 | 2492 | 3860 | n/a | n/a |
| BAVS01000030.1; | | | | | |
| 2161; 2162; 19/20 | | | | | |
| 1082; 427705465; | 1637 | 2759 | 4047 | n/a | n/a |
| NC_019676.1; 2163; | | | | | |
| 2164; 21/22 | | | | | |
| 1083; 428303693; | 1639 | 2765 | 4054 | n/a | n/a |
| NC_019753.1; 2165; | | | | | |
| 2166; 15/16 | | | | | |
| 1084; 359367134; | 1578 | 3064 | 3969 | n/a | n/a |
| NZ_AFEJ01000154.1; | | | | | |
| 2167; 2168; 21/22 | | | | | |
| 1085; 359367134; | 1578 | 3064 | 3969 | n/a | n/a |
| NZ_AFEJ01000154.1; | | | | | |
| 2169; 2170; 21/22 | | | | | |
| 1086; 325957759; | 1614 | 2726 | 4012 | n/a | n/a |
| NC_015216.1; 2171; | | | | | |
| 2172; 21/22 | | | | | |
| 1087; 851140085; | 2111 | 3601 | 4456 | n/a | n/a |
| NZ_JQKN01000008.1; | | | | | |
| 2173; 2174; 21/22 | | | | | |
| 1088; 748181452; | 2014 | 3322 | 4370 | n/a | n/a |
| NZ_JTCM01000043.1; | | | | | |
| 2175; 2176; 21/22 | | | | | |
| 1089; 748181452; | 2014 | 3322 | 4370 | n/a | n/a |
| NZ_JTCM01000043.1; | | | | | |
| 2177; 2178; 21/22 | | | | | |
| 1090; 158333233; | 1595 | 2694 | 3990 | n/a | n/a |
| NC_009925.1; 2179; | | | | | |
| 2180; 21/22 | | | | | |
| 1091; 158333233; | 1595 | 2694 | 3990 | n/a | n/a |
| NC_009925.1; 2181; | | | | | |
| 2182; 21/22 | | | | | |
| 1092; 851114167; | 2232 | 3619 | 4455 | n/a | n/a |
| NZ_LN515531.1; 2183; | | | | | |
| 2184; 23/24 | | | | | |
| 1093; 952971377; | 1379 | 2426 | 3819 | n/a | n/a |
| LN734822.1; 2185; | | | | | |
| 2186; 25/26 | | | | | |
| 1094; 428267688; | n/a | 2372 | 3779 | n/a | n/a |
| CP003653.1; 2187; | | | | | |
| 2188; 22/23 | | | | | |
| 1095; 333986242; | 1617 | 2731 | 4017 | n/a | n/a |
| NC_015574.1; 2189; | | | | | |
| 2190; 24/25 | | | | | |
| 1096; 739419616; | 2178 | 3232 | 4490 | n/a | n/a |
| NZ_KK088564.1; 2191; | | | | | |
| 2192; 20/21 | | | | | |
| 1097; 739419616; | 2178 | 3232 | 4490 | n/a | n/a |
| NZ_KK088564.1; 2193; | | | | | |
| 2194; 31/32 | | | | | |
| 1098; 427727289; | 1638 | 2763 | 4052 | n/a | n/a |
| NC_019684.1; 2195; | | | | | |
| 2196; 21/22 | | | | | |
| 1099; 890002594; | 2121 | 3496 | 4466 | n/a | n/a |
| NZ_JXCA01000005.1; | | | | | |
| 2197; 2198; 21/22 | | | | | |
| 1100; 652337551; | 1788 | 3003 | 4194 | n/a | n/a |
| NZ_KI912149.1; 2199; | | | | | |
| 2200; 31/32 | | | | | |
| 1101; 427415532; | 1535 | 2624 | 3937 | n/a | n/a |
| NZ_JH993797.1; 2201; | | | | | |
| 2202; 22/23 | | | | | |
| 1102; 551035505; | 1736 | 2901 | n/a | n/a | n/a |
| NZ_ATVS01000030.1; | | | | | |
| 2203; 2204; 20/21 | | | | | |
| 1103; 553740975; | 2172 | 2907 | 4145 | n/a | n/a |
| NZ_AWNH01000084.1; | | | | | |
| 2205; 2206; 22/23 | | | | | |
| 1104; 851351157; | 2112 | 3483 | 4457 | n/a | n/a |
| NZ_JQLY01000001.1; | | | | | |
| 2207; 2208; 25/26 | | | | | |
| 1105; 485067373; | 1713 | 2868 | 4113 | n/a | n/a |
| NZ_KB217478.1; 2209; | | | | | |
| 2210; 58/59 | | | | | |
| 1106; 451945650; | 1341 | 2373 | 3780 | n/a | n/a |
| NC_020304.1; 2211; | | | | | |
| 2212; 36/37 | | | | | |
| 1107; 938259025; | 1478 | 2552 | 3892 | n/a | n/a |
| LJSW01000006.1; 2213; | | | | | |
| 2214; 25/26 | | | | | |
| 1108; 557371823; | 1741 | 3517 | 4473 | n/a | n/a |
| NZ_ASGZ01000002.1; | | | | | |
| 2215; 2216; 26/27 | | | | | |
| 1109; 336251750; | 1619 | 2735 | 4020 | n/a | n/a |
| NC_015658.1; 2217; | | | | | |
| 2218; 26/27 | | | | | |
| 1110; 557371823; | 1418 | 2472 | 3850 | n/a | n/a |
| NZ_ASGZ01000002.1; | | | | | |
| 2219; 2220; 26/27 | | | | | |
| 1111; 484104632; | 1689 | 2839 | 4098 | n/a | n/a |
| NZ_KB235948.1; 2221; | | | | | |
| 2222; 32/33 | | | | | |
| 1112; 484104632; | 1689 | 2839 | 4098 | n/a | n/a |
| NZ_KB235948.1; 2223; | | | | | |
| 2224; 32/33 | | | | | |
| 1113; 448406329; | 1537 | 2627 | 3941 | n/a | n/a |
| NZ_AOIU01000004.1; | | | | | |
| 2225; 2226; 24/25 | | | | | |
| 1114; 751565075; | 2025 | 3345 | 4383 | n/a | n/a |
| NZ_JXCB01000004.1; | | | | | |
| 2227; 2228; 21/22 | | | | | |
| 1115; 119943794; | 2034 | 2688 | 3984 | n/a | n/a |
| NC_008709.1; 2229; | | | | | |
| 2230; 38/39 | | | | | |
| 1116; 563938926; | 2319 | 3741 | 4575 | n/a | n/a |
| NZ_AYWX01000007.1; | | | | | |
| 2231; 2232; 26/27 | | | | | |
| 1117; 451945650; | 1642 | 3367 | 4508 | n/a | n/a |
| NC_020304.1; 2233; | | | | | |
| 2234; 24/25 | | | | | |
| 1118; 563938926; | 2319 | 3735 | 4575 | n/a | n/a |
| NZ_AYWX01000007.1; | | | | | |
| 2235; 2236; 26/27 | | | | | |
| 1119; 655133038; | 1826 | 3048 | n/a | n/a | n/a |
| NZ_AUCV01000014.1; | | | | | |
| 2237; 2238; 32/33 | | | | | |
| 1120; 947704650; | 2316 | 3731 | 4572 | n/a | n/a |
| NZ_LMID01000016.1; | | | | | |
| 2239; 2240; 22/23 | | | | | |
| 1121; 294505815; | 2153 | 2710 | 4001 | n/a | n/a |
| NC_014032.1; 2241; | | | | | |
| 2242; 21/22 | | | | | |
| 1122; 294505815; | 2153 | 2710 | 4001 | n/a | n/a |
| NC_014032.1; 2243; | | | | | |
| 2244; 18/19 | | | | | |
| 1123; 947919015; | 2318 | 3734 | 4574 | n/a | n/a |
| NZ_LMHP01000012.1; | | | | | |
| 2245; 2246; 26/27 | | | | | |
| 1124; 780791108; | n/a | 2518 | 3869 | n/a | n/a |
| LADS01000058.1; 2247; | | | | | |
| 2248; 22/23 | | | | | |
| 1125; 738999090; | 2176 | 3226 | 4342 | n/a | n/a |
| NZ_KK073873.1; 2249; | | | | | |
| 2250; 26/27 | | | | | |
| 1126; 408381849; | 1519 | 2604 | 3927 | n/a | n/a |
| NZ_AMPO01000004.1; | | | | | |
| 2251; 2252; 28/29 | | | | | |
| 1127; 338209545; | n/a | 2738 | n/a | n/a | n/a |
| NC_015703.1; 2253; | | | | | |
| 2254; 33/34 | | | | | |
| 1128; 294505815; | 2153 | 2710 | 4001 | n/a | n/a |
| NC_014032.1; 2255; | | | | | |
| 2256; 19/20 | | | | | |
| 1129; 294505815; | 2153 | 2710 | 4001 | n/a | n/a |
| NC_014032.1; 2257; | | | | | |
| 2258; 18/19 | | | | | |
| 1130; 427705465; | n/a | 2370 | 3777 | n/a | n/a |
| NC_019676.1; 2259; | | | | | |
| 2260; 35/36 | | | | | |
| 1131; 427705465; | n/a | 3493 | 4046 | n/a | n/a |
| NC_019676.1; 2261; | | | | | |
| 2262; 35/36 | | | | | |
| 1132; 640169055; | 1757 | 2958 | 4487 | n/a | n/a |
| NZ_JAFS01000002.1; | | | | | |
| 2263; 2264; 40/41 | | | | | |
| 1133; 943897669; | 2298 | 3707 | 4550 | n/a | n/a |
| NZ_LIQQ01000007.1; | | | | | |
| 2265; 2266; 21/22 | | | | | |
| 1134; 943674269; | 2296 | 3705 | 4548 | n/a | n/a |
| NZ_LIQO01000205.1; | | | | | |
| 2267; 2268; 21/22 | | | | | |
| 1135; 386348020; | 1587 | 2680 | 3978 | n/a | n/a |
| NC_017584.1; 2269; | | | | | |
| 2270; 36/37 | | | | | |
| 1136; 931421682; | 1473 | 2547 | 3889 | n/a | n/a |
| LJTQ01000030.1; 2271; | | | | | |
| 2272; 29/30 | | | | | |
| 1137; 890444402; | 2122 | 3497 | 4467 | n/a | n/a |
| NZ_CP011310.1; 2273; | | | | | |
| 2274; 30/31 | | | | | |
| 1138; 41582259; | 1316 | 2337 | n/a | n/a | n/a |
| AY458641.2; 2275; | | | | | |
| 2276; 42/43 | | | | | |
| 1139; 41582259; | 2021 | 2631 | n/a | n/a | n/a |
| AY458641.2; 2277; | | | | | |
| 2278; 42/43 | | | | | |
| 1140; 554634310; | n/a | 3555 | 4147 | n/a | n/a |
| NC_022600.1; 2279; | | | | | |
| 2280; 28/29 | | | | | |
| 1141; 947721816; | 2317 | 3732 | 4573 | n/a | n/a |
| NZ_LMIB01000001.1; | | | | | |
| 2281; 2282; 22/23 | | | | | |
| 1142; 554634310; | n/a | 2377 | 3784 | n/a | n/a |
| NC_022600.1; 2283; | | | | | |
| 2284; 28/29 | | | | | |
| 1143; 483724571; | n/a | 2854 | 4106 | n/a | n/a |
| NZ_KB904821.1; 2285; | | | | | |
| 2286; 26/27 | | | | | |
| 1144; 557835508; | 1743 | 2911 | 4149 | n/a | n/a |
| NZ_AWGE01000033.1; | | | | | |
| 2287; 2288; 25/26 | | | | | |
| 1145; 575082509; | 1432 | 2492 | 3860 | n/a | n/a |
| BAVS01000030.1; | | | | | |
| 2289; 2290; 19/20 | | | | | |
| 1146; 553739852; | 1906 | 2905 | 4143 | n/a | n/a |
| NZ_AWNH01000066.1; | | | | | |
| 2291; 2292; 33/34 | | | | | |
| 1147; 484345004; | 1667 | 2806 | 4078 | n/a | n/a |
| NZ_JH947126.1; 2293; | | | | | |
| 2294; 30/31 | | | | | |
| 1148; 482909235; | n/a | 2808 | n/a | n/a | n/a |
| NZ_JH980292.1; 2295; | | | | | |
| 2296; 32/33 | | | | | |
| 1149; 737370143; | 1947 | 3200 | 4330 | n/a | n/a |
| NZ_JQKI01000040.1; | | | | | |
| 2297; 2298; 18/19 | | | | | |
| 1150; 734983081; | n/a | 3180 | n/a | n/a | n/a |
| NZ_JSXI01000073.1; | | | | | |
| 2299; 2300; 24/25 | | | | | |
| 1151; 736965849; | 1941 | 3189 | 4324 | n/a | n/a |
| NZ_JMIW01000009.1; | | | | | |
| 2301; 2302; 26/27 | | | | | |
| 1152; 483219562; | 1697 | 2849 | 4103 | n/a | n/a |
| NZ_KB901875.1; 2303; | | | | | |
| 2304; 38/39 | | | | | |
| 1153; 326793322; | 1615 | 2727 | 4013 | n/a | n/a |
| NC_015276.1; 2305; | | | | | |
| 2306; 40/41 | | | | | |
| 1154; 347753732; | 1626 | 2744 | 4027 | n/a | n/a |
| NC_016024.1; 2307; | | | | | |
| 2308; 41/42 | | | | | |
| 1155; 947472882; | 2312 | 3726 | 4566 | n/a | n/a |
| NZ_LMRH01000002.1; | | | | | |
| 2309; 2310; 21/22 | | | | | |
| 1156; 953813788; | n/a | 3748 | n/a | n/a | n/a |
| NZ_LNBE01000002.1; | | | | | |
| 2311; 2312; 12/13 | | | | | |
| 1157; 943922224; | 2301 | 3710 | 4553 | n/a | n/a |
| NZ_LIQU01000122.1; | | | | | |
| 2313; 2314; 12/13 | | | | | |
| 1158; 944029528; | 2306 | 3717 | 4560 | n/a | n/a |
| NZ_LIQZ01000126.1; | | | | | |
| 2315; 2316; 12/13 | | | | | |
| 1159; 943898694; | 2299 | 3708 | 4551 | n/a | n/a |
| NZ_LIQN01000037.1; | | | | | |
| 2317; 2318; 19/20 | | | | | |
| 1160; 953813789; | n/a | 3749 | n/a | n/a | n/a |
| NZ_LNBE01000003.1; | | | | | |
| 2319; 2320; 49/50 | | | | | |
| 1161; 943881150; | 2297 | 3706 | 4549 | n/a | n/a |
| NZ_LIPP01000138.1; | | | | | |
| 2321; 2322; 35/36 | | | | | |
| 1162; 943927948; | 2302 | 3712 | 4555 | n/a | n/a |
| NZ_LIQV01000315.1; | | | | | |
| 2323; 2324; 24/25 | | | | | |
| 1163; 943949281; | 2303 | 3713 | 4556 | n/a | n/a |
| NZ_LIPN01000124.1; | | | | | |
| 2325; 2326; 21/22 | | | | | |
| 1164; 951121600; | 2327 | 3746 | 4585 | n/a | n/a |
| NZ_LMEQ01000031.1; | | | | | |
| 2327; 2328; 21/22 | | | | | |
| 1165; 944495433; | 2307 | 3720 | 4563 | n/a | n/a |
| NZ_LIRK01000018.1; | | | | | |
| 2329; 2330; 21/22 | | | | | |
| 1166; 943899498; | 2300 | 3709 | 4552 | n/a | n/a |
| NZ_LIQN01000384.1; | | | | | |
| 2331; 2332; 21/22 | | | | | |
| 1167; 483258918; | 1392 | 2443 | 3830 | n/a | n/a |
| NZ_AMFE01000033.1; | | | | | |
| 2333; 2334; 19/20 | | | | | |
| 1168; 483258918; | 1392 | 2443 | 3830 | n/a | n/a |
| NZ_AMFE01000033.1; | | | | | |
| 2335; 2336; 19/20 | | | | | |
| 1169; 944012845; | 2305 | 3715 | 4558 | n/a | n/a |
| NZ_LIPQ01000171.1; | | | | | |
| 2337; 2338; 40/41 | | | | | |
| 1170; 664052786; | 1874 | 3097 | 4270 | n/a | n/a |
| NZ_JOES01000014.1; | | | | | |
| 2339; 2340; 21/22 | | | | | |
| 1171; 652876473; | n/a | 2634 | 3947 | n/a | n/a |
| NZ_KI912267.1; 2341; | | | | | |
| 2342; 34/35 | | | | | |
| 1172; 959926096; | 1815 | 3036 | 4337 | n/a | n/a |
| NZ_LMTZ01000085.1; | | | | | |
| 2343; 2344; 21/22 | | | | | |
| 1173; 959868240; | 2329 | 3751 | 4165 | n/a | n/a |
| NZ_CP013252.1; 2345; | | | | | |
| 2346; 18/19 | | | | | |
| 1174; 483254584; | 2157 | 2881 | 4127 | n/a | n/a |
| NZ_KB902362.1; 2347; | | | | | |
| 2348; 42/43 | | | | | |
| 1175; 655990125; | 1831 | 3600 | 4510 | n/a | n/a |
| NZ_AUBC01000024.1; | | | | | |
| 2349; 2350; 26/27 | | | | | |
| 1176; 746187665; | 2219 | 3305 | 4365 | n/a | n/a |
| NZ_JWSY01000013.1; | | | | | |
| 2351; 2352; 12/13 | | | | | |
| 1177; 443625867; | 1518 | 2603 | 4356 | n/a | n/a |
| NZ_AMLP01000127.1; | | | | | |
| 2353; 2354; 20/21 | | | | | |
| 1178; 386284588; | 1551 | 2641 | 3952 | n/a | n/a |
| NZ_AJLE01000006.1; | | | | | |
| 2355; 2356; 26/27 | | | | | |
| 1179; 826051019; | 2244 | 3631 | 4446 | n/a | n/a |
| NZ_LDES01000074.1; | | | | | |
| 2357; 2358; 22/23 | | | | | |
| 1180; 312128809; | n/a | 2718 | n/a | n/a | n/a |
| NC_014655.1; 2359; | | | | | |
| 2360; 25/26 | | | | | |
| 1181; 482849861; | 1506 | 2589 | 3920 | n/a | n/a |
| NZ_AKBU01000001.1; | | | | | |
| 2361; 2362; 3/4 | | | | | |
| 1182; 879201007; | 1380 | 2427 | 3820 | n/a | n/a |
| CKIK01000005.1; 2363; | | | | | |
| 2364; 19/20 | | | | | |
| 1183; 482849861; | 1585 | 2677 | 3963 | n/a | n/a |
| NZ_AKBU01000001.1; | | | | | |
| 2365; 2366; 3/4 | | | | | |
| 1184; 835319962; | 2213 | 3474 | 4447 | n/a | n/a |
| NZ_JTLD01000119.1; | | | | | |
| 2367; 2368; 22/23 | | | | | |
| 1185; 766607514; | 1839 | 3426 | 4421 | n/a | n/a |
| NZ_JTHO01000003.1; | | | | | |
| 2369; 2370; 20/21 | | | | | |
| 1186; 671525382; | n/a | 3130 | 4496 | n/a | n/a |
| NZ_JODL01000019.1; | | | | | |
| 2371; 2372; 31/32 | | | | | |
| 1187; 146276058; | 1591 | 2691 | 3986 | n/a | n/a |
| NC_009428.1; 2373; | | | | | |
| 2374; 32/33 | | | | | |
| 1188; 563938926; | 1620 | 2736 | 4021 | n/a | n/a |
| NZ_AYWX01000007.1; | | | | | |
| 2375; 2376; 26/27 | | | | | |
| 1189; 739662450; | n/a | n/a | n/a | n/a | n/a |
| NZ_JNFD01000038.1; | | | | | |
| 2377; 2378; 20/21 | | | | | |
| 1190; 739662450; | 1444 | n/a | n/a | n/a | n/a |
| NZ_JNFD01000038.1; | | | | | |
| 2379; 2380; 20/21 | | | | | |
| 1191; 906292938; | 1740 | 2909 | n/a | n/a | n/a |
| CXPB01000073.1; 2381; | | | | | |
| 2382; 18/19 | | | | | |
| 1192; 653556699; | 1813 | 3034 | n/a | n/a | n/a |
| NZ_AUEZ01000087.1; | | | | | |
| 2383; 2384; 26/27 | | | | | |
| 1193; 844809159; | 2107 | 3479 | 4450 | n/a | n/a |
| NZ_LDPH01000011.1; | | | | | |
| 2385; 2386; 20/21 | | | | | |
| 1194; 483961722; | n/a | 2988 | n/a | n/a | n/a |
| NZ_KB890915.1; 2387; | | | | | |
| 2388; 71/72 | | | | | |
| 1195; 739487309; | n/a | 3235 | n/a | n/a | 4504 |
| NZ_JPLW01000007.1; | | | | | |
| 2389; 2390; 27/28 | | | | | |
| 1196; 921170702; | 1884 | 3456 | n/a | n/a | n/a |
| NZ_CP009922.2; 2391; | | | | | |
| 2392; 13/14 | | | | | |
| 1197; 644043488; | 1764 | 3202 | 4174 | n/a | n/a |
| NZ_AZUQ01000001.1; | | | | | |
| 2393; 2394; 19/20 | | | | | |
| 1198; 921170702; | 1356 | 2390 | n/a | n/a | n/a |
| NZ_CP009922.2; 2395; | | | | | |
| 2396; 13/14 | | | | | |
| 1199; 254392242; | 1513 | 2598 | 3922 | n/a | n/a |
| NZ_DS570678.1; 2397; | | | | | |
| 2398; 39/40 | | | | | |
| 1200; 483975550; | 2158 | 3263 | n/a | n/a | n/a |
| NZ_KB892001.1; 2399; | | | | | |
| 2400; 30/31 | | | | | |
| 1201; 550281965; | n/a | 3336 | n/a | n/a | n/a |
| NZ_ASSJ01000070.1; | | | | | |
| 2401; 2402; 27/28 | | | | | |
| 1202; 291297538; | 1330 | 2355 | n/a | n/a | n/a |
| NC_013947.1; 2403; | | | | | |
| 2404; 29/30 | | | | | |
| 1203; 662129456; | n/a | 3532 | n/a | n/a | n/a |
| NZ_KL573544.1; 2405; | | | | | |
| 2406; 28/29 | | | | | |
| 1204; 291297538; | 1606 | 3362 | 4389 | n/a | n/a |
| NC_013947.1; 2407; | | | | | |
| 2408; 29/30 | | | | | |
| 1205; 484015294; | 1777 | 2826 | 4091 | n/a | n/a |
| NZ_ANAX01000026.1; | | | | | |
| 2409; 2410; 29/30 | | | | | |
| 1206; 655370026; | 2166 | 3051 | 4223 | n/a | n/a |
| NZ_ATZF01000001.1; | | | | | |
| 2411; 2412; 21/22 | | | | | |
| 1207; 484016825; | n/a | 2827 | n/a | n/a | n/a |
| NZ_ANAY01000003.1; | | | | | |
| 2413; 2414; 22/23 | | | | | |
| 1208; 926283036; | n/a | 3650 | n/a | n/a | n/a |
| NZ_LGEC01000103.1; | | | | | |
| 2415; 2416; 66/67 | | | | | |
| 1209; 408675720; | 1636 | 2757 | n/a | n/a | n/a |
| NC_018750.1; 2417; | | | | | |
| 2418; 27128 | | | | | |
| 1210; 254387191; | 1554 | 3634 | n/a | n/a | n/a |
| NZ_DS570483.1; 2419; | | | | | |
| 2420; 27/28 | | | | | |
| 1211; 772744565; | n/a | 2517 | 3868 | n/a | n/a |
| NZ_JYJG01000059.1; | | | | | |
| 2421; 2422; 33/34 | | | | | |
| 1212; 919531973; | 2243 | 3627 | 4519 | n/a | n/a |
| NZ_JOEK01000003.1; | | | | | |
| 2423; 2424; 25/26 | | | | | |
| 1213; 671498318; | 2194 | 3580 | n/a | n/a | n/a |
| NZ_JOFR01000042.1; | | | | | |
| 2425; 2426; 23/24 | | | | | |
| 1214; 671498318; | 2194 | 3580 | n/a | n/a | n/a |
| NZ_JOFR01000042.1; | | | | | |
| 2427; 2428; 34/35 | | | | | |
| 1215; 514917321; | 1660 | 2796 | 4072 | n/a | n/a |
| NZ_AOPZ01000063.1; | | | | | |
| 2429; 2430; 37/38 | | | | | |
| 1216; 739097522; | 2174 | 3227 | n/a | n/a | n/a |
| NZ_KI911740.1; 2431; | | | | | |
| 2432; 28/29 | | | | | |
| 1217; 665618015; | 2187 | 3567 | 4310 | n/a | n/a |
| NZ_JODR01000032.1; | | | | | |
| 2433; 2434; 40/41 | | | | | |
| 1218; 926412094; | n/a | 3662 | n/a | n/a | 4532 |
| NZ_LGDY01000103.1; | | | | | |
| 2435; 2436; 30/31 | | | | | |
| 1219; 935540718; | n/a | 2544 | n/a | n/a | n/a |
| NZ_LGJH01000063.1; | | | | | |
| 2437; 2438; 23/24 | | | | | |
| 1220; 665536304; | 2195 | 3582 | 4297 | n/a | n/a |
| NZ_JOCD01000152.1; | | | | | |
| 2439; 2440; 35/36 | | | | | |
| 1221; 665618015; | 2187 | 3564 | 4310 | n/a | n/a |
| NZ_JODR01000032.1; | | | | | |
| 2441; 2442; 40/41 | | | | | |
| 1222; 772744565; | n/a | 3431 | 4425 | n/a | n/a |
| NZ_JYJG01000059.1; | | | | | |
| 2443; 2444; 33/34 | | | | | |
| 1223; 483112234; | 2212 | 2798 | n/a | n/a | n/a |
| NZ_AGVX02000406.1; | | | | | |
| 2445; 2446; 24/25 | | | | | |
| 1224; 739372122; | n/a | n/a | 3865 | n/a | n/a |
| NZ_JQHE01000003.1; | | | | | |
| 2447; 2448; 11/12 | | | | | |
| 1225; 739372122; | n/a | n/a | 3865 | n/a | n/a |
| NZ_JQHE01000003.1; | | | | | |
| 2449; 2450; 13/14 | | | | | |
| 1226; 664360925; | 2197 | 3114 | 4285 | n/a | n/a |
| NZ_JOGD01000054.1; | | | | | |
| 2451; 2452; 25/26 | | | | | |
| 1227; 358468594; | n/a | 2669 | n/a | n/a | n/a |
| NZ_FR873693.1; 2453; | | | | | |
| 2454; 14/15 | | | | | |
| 1228; 358468594; | n/a | 2669 | n/a | n/a | n/a |
| NZ_FR873693.1; 2455; | | | | | |
| 2456; 26/27 | | | | | |
| 1229; 358468601; | 1580 | 2670 | n/a | n/a | n/a |
| NZ_FR873700.1; 2457; | | | | | |
| 2458; 69/70 | | | | | |
| 1230; 663199697; | n/a | 3082 | n/a | n/a | n/a |
| NZ_JOHO01000012.1; | | | | | |
| 2459; 2460; 30/31 | | | | | |
| 1231; 665671804; | 2145 | 3538 | 4308 | n/a | n/a |
| NZ_JOCK01000052.1; | | | | | |
| 2461; 2462; 40/41 | | | | | |
| 1232; 254387191; | 1388 | 2436 | n/a | n/a | n/a |
| NZ_DS570483.1; 2463; | | | | | |
| 2464; 27/28 | | | | | |
| 1233; 224581098; | 1557 | 2648 | n/a | n/a | n/a |
| NZ_GG657748.1; 2465; | | | | | |
| 2466; 35/36 | | | | | |
| 1234; 110677421; | 1589 | 2685 | 3982 | n/a | n/a |
| NC_008209.1; 2467; | | | | | |
| 2468; 22/23 | | | | | |
| 1235; 563312125; | 1588 | 2682 | n/a | n/a | n/a |
| AYTZ01000052.1; | | | | | |
| 2469; 2470; 31/32 | | | | | |
| 1236; 935540718; | n/a | 3686 | n/a | n/a | n/a |
| NZ_LGJH01000063.1; | | | | | |
| 2471; 2472; 23/24 | | | | | |
| 1237; 326336949; | n/a | 2659 | n/a | n/a | n/a |
| NZ_CM001018.1; 2473; | | | | | |
| 2474; 35/36 | | | | | |
| 1238; 663670981; | n/a | 3092 | n/a | n/a | 4262 |
| NZ_JODQ01000007.1; | | | | | |
| 2475; 2476; 20/21 | | | | | |
| 1239; 546154317; | n/a | n/a | n/a | n/a | n/a |
| NZ_ACVN02000045.1; | | | | | |
| 2477; 2478; 18/19 | | | | | |
| 1240; 563312125; | 1588 | 3211 | n/a | n/a | n/a |
| AYTZ01000052.1; | | | | | |
| 2479; 2480; 31/32 | | | | | |
| 1241; 483258918; | 1392 | 2443 | 3830 | n/a | n/a |
| NZ_AMFE01000033.1; | | | | | |
| 2481; 2482; 19/20 | | | | | |
| 1242; 483258918; | 1392 | 2443 | 3830 | n/a | n/a |
| NZ_AMFE01000033.1; | | | | | |
| 2483; 2484; 19/20 | | | | | |
| 1243; 820820518; | 2237 | 3624 | n/a | n/a | n/a |
| NZ_KQ061219.1; 2485; | | | | | |
| 2486; 31/32 | | | | | |
| 1244; 514348304; | 1657 | 2795 | n/a | n/a | n/a |
| NZ_ASQH01000001.1; | | | | | |
| 2487; 2488; 26/27 | | | | | |
| 1245; 928675838; | 1386 | 2434 | n/a | n/a | n/a |
| CYTQ01000003.1; | | | | | |
| 2489; 2490; 27/28 | | | | | |
| 1246; 652698054; | 1793 | 3009 | 4198 | n/a | n/a |
| NZ_KI912610.1; 2491; | | | | | |
| 2492; 26/27 | | | | | |
| 1247; 759875025; | n/a | 3400 | n/a | n/a | n/a |
| NZ_JONS01000016.1; | | | | | |
| 2493; 2494; 12/13 | | | | | |
| 1248; 664141438; | n/a | 3584 | n/a | n/a | n/a |
| NZ_JOJM01000019.1; | | | | | |
| 2495; 2496; 29/30 | | | | | |
| 1249; 483258918; | 1392 | 2443 | 3830 | n/a | n/a |
| NZ_AMFE01000033.1; | | | | | |
| 2497; 2498; 19/20 | | | | | |
| 1250; 483258918; | 1392 | 2443 | 3830 | n/a | n/a |
| NZ_AMFE01000033.1; | | | | | |
| 2499; 2500; 19/20 | | | | | |
| 1251; 929862756; | 1732 | 2897 | 4137 | n/a | n/a |
| NZ_LGKI01000090.1; | | | | | |
| 2501; 2502; 27/28 | | | | | |
| 1252; 378759075; | 1575 | 2664 | 3966 | n/a | n/a |
| NZ_AFXE01000029.1; | | | | | |
| 2503; 2504; 22/23 | | | | | |
| 1253; 484005069; | n/a | 3551 | n/a | n/a | n/a |
| NZ_KB894416.1; 2505; | | | | | |
| 2506; 18/19 | | | | | |
| 1254; 563478461; | n/a | 2932 | 4154 | n/a | n/a |
| NZ_AYVQ01000029.1; | | | | | |
| 2507; 2508; 30/31 | | | | | |
| 1255; 482984722; | 1780 | 2848 | n/a | n/a | n/a |
| NZ_KB900605.1; 2509; | | | | | |
| 2510; 23/24 | | | | | |
| 1256; 563478461; | n/a | 2923 | 4156 | n/a | n/a |
| NZ_AYVQ01000029.1; | | | | | |
| 2511; 2512; 30/31 | | | | | |
| 1257; 563478461; | n/a | 2920 | 4156 | n/a | n/a |
| NZ_AYVQ01000029.1; | | | | | |
| 2513; 2514; 30/31 | | | | | |
| 1258; 563478461; | n/a | 2917 | 4154 | n/a | n/a |
| NZ_AYVQ01000029.1; | | | | | |
| 2515; 2516; 30/31 | | | | | |
| 1259; 563478461; | n/a | 2940 | 4161 | n/a | n/a |
| NZ_AYVQ01000029.1; | | | | | |
| 2517; 2518; 30/31 | | | | | |
| 1260; 563478461; | n/a | 2924 | 4158 | n/a | n/a |
| NZ_AYVQ01000029.1; | | | | | |
| 2519; 2520; 30/31 | | | | | |
| 1261;563478461; | n/a | 2933 | 4154 | n/a | n/a |
| NZ_AYVQ01000029.1; | | | | | |
| 2521; 2522; 30/31 | | | | | |
| 1262; 563478461; | n/a | 2926 | 4156 | n/a | n/a |
| NZ_AYVQ01000029.1; | | | | | |
| 2523; 2524; 30/31 | | | | | |
| 1263; 563312125; | 1426 | 2482 | n/a | n/a | n/a |
| AYTZ01000052.1; | | | | | |
| 2525; 2526; 31/32 | | | | | |
| 1264; 563478461; | n/a | 2928 | 4154 | n/a | n/a |
| NZ_AYVQ01000029.1; | | | | | |
| 2527; 2528; 30/31 | | | | | |
| 1265; 652698054; | 1800 | 3014 | 4202 | n/a | n/a |
| NZ_KI912610.1; 2529; | | | | | |
| 2530; 26/27 | | | | | |
| 1266; 652698054; | 1796 | 3011 | 4200 | n/a | n/a |
| NZ_KI912610.1; 2531; | | | | | |
| 2532; 26/27 | | | | | |
| 1267; 484023389; | 2154 | 2832 | n/a | n/a | n/a |
| NZ_ANBF01000087.1; | | | | | |
| 2533; 2534; 24/25 | | | | | |
| 1268; 655569633; | 1971 | 3057 | 4491 | n/a | n/a |
| NZ_JIAI01000002.1; | | | | | |
| 2535; 2536; 32/33 | | | | | |
| 1269; 655569633; | 1971 | 3057 | 4491 | n/a | n/a |
| NZ_JIAI01000002.1; | | | | | |
| 2537; 2538; 43/44 | | | | | |
| 1270; 655569633; | 1971 | 3057 | 4491 | n/a | n/a |
| NZ_JIAI01000002.1; | | | | | |
| 2539; 2540; 32/33 | | | | | |
| 1271; 563478461; | n/a | 2925 | 4158 | n/a | n/a |
| NZ_AYVQ01000029.1; | | | | | |
| 2541; 2542; 30/31 | | | | | |
| 1272; 740292158; | 2186 | 3276 | 4361 | n/a | n/a |
| NZ_AUNB01000028.1; | | | | | |
| 2543; 2544; 22/23 | | | | | |
| 1273; 563478461; | n/a | 2921 | 4157 | n/a | n/a |
| NZ_AYVQ01000029.1; | | | | | |
| 2545; 2546; 30/31 | | | | | |
| 1274; 563478461; | n/a | 2930 | 4154 | n/a | n/a |
| NZ_AYVQ01000029.1; | | | | | |
| 2547; 2548; 30/31 | | | | | |
| 1275; 563478461; | n/a | 2927 | 4154 | n/a | n/a |
| NZ_AYVQ01000029.1; | | | | | |
| 2549; 2550; 30/31 | | | | | |
| 1276; 563478461; | n/a | 2918 | 4155 | n/a | n/a |
| NZ_AYVQ01000029.1; | | | | | |
| 2551; 2552; 30/31 | | | | | |
| 1277; 740220529; | 2185 | 3274 | 4495 | n/a | n/a |
| NZ_JHEH01000002.1; | | | | | |
| 2553; 2554; 13/14 | | | | | |
| 1278; 563478461; | n/a | 2919 | 4154 | n/a | n/a |
| NZ_AYVQ01000029.1; | | | | | |
| 2555; 2556; 30/31 | | | | | |
| 1279; 483454700; | 1722 | 2987 | 4128 | n/a | n/a |
| NZ_KB903974.1; 2557; | | | | | |
| 2558; 31/32 | | | | | |
| 1280; 835355240; | 2103 | 3475 | n/a | n/a | n/a |
| NZ_KN549147.1; 2559; | | | | | |
| 2560; 13/14 | | | | | |
| 1281; 563478461; | n/a | 2929 | 4154 | n/a | n/a |
| NZ_AYVQ01000029.1; | | | | | |
| 2561; 2562; 30/31 | | | | | |
| 1282; 563478461; | n/a | 2944 | 4158 | n/a | n/a |
| NZ_AYVQ01000029.1; | | | | | |
| 2563; 2564; 30/31 | | | | | |
| 1283; 652698054; | 1921 | 3158 | 3972 | n/a | n/a |
| NZ_KI912610.1; 2565; | | | | | |
| 2566; 26/27 | | | | | |
| 1284; 563478461; | n/a | 2931 | 4154 | n/a | n/a |
| NZ_AYVQ01000029.1; | | | | | |
| 2567; 2568; 30/31 | | | | | |
| 1285; 563478461; | n/a | 2943 | 4154 | n/a | n/a |
| NZ_AYVQ01000029.1; | | | | | |
| 2569; 2570; 30/31 | | | | | |
| 1286; 652879634; | 1802 | 3019 | 4204 | n/a | n/a |
| NZ_AZUY01000007.1; | | | | | |
| 2571; 2572; 26/27 | | | | | |
| 1287; 652698054; | 1795 | 3010 | 4199 | n/a | n/a |
| NZ_KI912610.1; 2573; | | | | | |
| 2574; 26/27 | | | | | |
| 1288; 563478461; | n/a | 2922 | 4154 | n/a | n/a |
| NZ_AYVQ01000029.1; | | | | | |
| 2575; 2576; 30/31 | | | | | |
| 1289; 652698054; | 1803 | 3020 | 4205 | n/a | n/a |
| NZ_KI912610.1; 2577; | | | | | |
| 2578; 26/27 | | | | | |
| 1290; 563478461; | n/a | 3012 | 4154 | n/a | n/a |
| NZ_AYVQ01000029.1; | | | | | |
| 2579; 2580; 30/31 | | | | | |
| 1291; 563478461; | n/a | 2945 | 4154 | n/a | n/a |
| NZ_AYVQ01000029.1; | | | | | |
| 2581; 2582; 30/31 | | | | | |
| 1292; 652698054; | 1582 | 2673 | 3972 | n/a | n/a |
| NZ_KI912610.1; 2583; | | | | | |
| 2584; 26/27 | | | | | |
| 1293; 563478461; | n/a | 2942 | 4154 | n/a | n/a |
| NZ_AYVQ01000029.1; | | | | | |
| 2585; 2586; 30/31 | | | | | |
| 1294; 652698054; | 1798 | 3013 | 4201 | n/a | n/a |
| NZ_KI912610.1; 2587; | | | | | |
| 2588; 26/27 | | | | | |
| 1295; 563938926; | 2147 | 2941 | 4162 | n/a | n/a |
| NZ_AYWX01000007.1; | | | | | |
| 2589; 2590; 26/27 | | | | | |
| 1296; 483314733; | 1699 | 2851 | n/a | n/a | n/a |
| NZ_KB902785.1; 2591; | | | | | |
| 2592; 13/14 | | | | | |
| 1297; 652698054; | 1716 | 2875 | 4120 | n/a | n/a |
| NZ_KI912610.1; 2593; | | | | | |
| 2594; 26/27 | | | | | |
| 1298; 652698054; | 1920 | 2954 | 4009 | n/a | n/a |
| NZ_KI912610.1; 2595; | | | | | |
| 2596; 26/27 | | | | | |
| 1299; 652670206; | 1791 | 3008 | 4197 | n/a | n/a |
| NZ_AUEL01000005.1; | | | | | |
| 2597; 2598; 26/27 | | | | | |
| 1300; 657698352; | 1739 | 2908 | n/a | n/a | n/a |
| NZ_JDWO01000067.1; | | | | | |
| 2599; 2600; 25/26 | | | | | |
| 1301; 653526890; | 1961 | 3033 | n/a | n/a | n/a |
| NZ_AXAZ01000002.1; | | | | | |
| 2601; 2602; 26/27 | | | | | |
| 1302; 433771415; | 1749 | 2937 | 4056 | n/a | n/a |
| NC_019973.1; 2603; | | | | | |
| 2604; 26/27 | | | | | |
| 1303; 433771415; | 1749 | 2938 | 4056 | n/a | n/a |
| NC_019973.1; 2605; | | | | | |
| 2606; 26/27 | | | | | |
| 1304; 433771415; | 1641 | 2768 | 4056 | n/a | n/a |
| NC_019973.1; 2607; | | | | | |
| 2608; 26/27 | | | | | |
| 1305; 657698352; | 1739 | 3069 | n/a | n/a | n/a |
| NZ_JDWO01000067.1; | | | | | |
| 2609; 2610; 25/26 | | | | | |
| 1306; 339501577; | 1622 | 2739 | 4023 | n/a | n/a |
| NC_015730.1; 2611; | | | | | |
| 2612; 22/23 | | | | | |
| 1307; 639168743; | 1755 | 2955 | n/a | n/a | n/a |
| NZ_AWZU01000010.1; | | | | | |
| 2613; 2614; 21/22 | | | | | |
| 1308; 433771415; | 1749 | 2935 | 4056 | n/a | n/a |
| NC_019973.1; 2615; | | | | | |
| 2616; 26/27 | | | | | |
| 1309; 484075173; | n/a | 2801 | n/a | n/a | 4076 |
| NZ_AJLK01000109.1; | | | | | |
| 2617; 2618; 27/28 | | | | | |
| 1310; 906292938; | 1384 | 2432 | n/a | n/a | n/a |
| CXPB01000073.1; 2619; | | | | | |
| 2620; 18/19 | | | | | |
| 1311; 652912253; | 1962 | 3021 | 4206 | n/a | n/a |
| NZ_ATYO01000004.1; | | | | | |
| 2621; 2622; 26/27 | | | | | |
| 1312; 906292938; | 2018 | 3332 | n/a | n/a | n/a |
| CXPB01000073.1; 2623; | | | | | |
| 2624; 18/19 | | | | | |
| 1313; 970574347; | 1768 | 2814 | 4084 | n/a | n/a |
| NZ_LNZF01000001.1; | | | | | |
| 2625; 2626; 20/21 | | | | | |
| 1314; 970574347; | 2001 | 3307 | 4074 | n/a | n/a |
| NZ_LNZF01000001.1; | | | | | |
| 2627; 2628; 20/21 | | | | | |
| 1315; 970574347; | 1768 | 3129 | 4084 | n/a | n/a |
| NZ_LNZF01000001.1; | | | | | |
| 2629; 2630; 20/21 | | | | | |

TABLE 3
Exemplary Lasso Peptidase
Lasso Peptidase Peptide No: #; Species of Origin; GI #; Accession #

1316; Uncultured marine bacterium 463 clone EBAC080-L32B05 genomic
sequence; 41582259; AY458641.2
1317; Burkholderia pseudomallei 1710b chromosome I, complete sequence;
76808520; NC_007434.1
1318; Burkholderia thailandensis E555 BTHE555_314, whole genome shotgun sequence;
485035557; NZ_AECN01000315.1
1319; Frankia sp. CcI6 CcI6DRAFT_scaffold_51.52, whole genome shotgun sequence;
563312125; AYTZ01000052.1
1320; Sphingopyxis alaskensis RB2256, complete genome; 103485498;
NC_008048.1
1321; Sphingopyxis alaskensis RB2256, complete genome; 103485498;
NC_008048.1
1322; Streptococcus suis SC84 complete genome, strain SC84; 253750923;
NC_012924.1
1323; Geobacter uraniireducens Rf4, complete genome; 148262085;
NC_009483.1
1324; Caulobacter sp. K31, complete genome; 167643973; NC_010338.1
1325; Phenylobacterium zucineum HLK1, complete genome; 196476886;
CP000747.1
1326; Phenylobacterium zucineum HLK1, complete genome; 196476886;
CP000747.1
1327; Sanguibacter keddieii DSM 10542, complete genome; 269793358;
NC_013521.1
1328; Xylanimonas cellulosilytica DSM 15894, complete genome; 269954810;
NC_013530.1
1329; Spirosoma linguale DSM 74, complete genome; 283814236; CP001769.1
1330; Stackebrandtia nassauensis DSM 44728, complete genome; 291297538;
NC_013947.1
1331; Caulobacter segnis ATCC 21756, complete genome; 295429362;
CP002008.1
1332; Streptomyces bingchenggensis BCW-1, complete genome; 374982757;
NC_016582.1
1333; Gallionella capsifeniformans ES-2, complete genome; 302877245;
NC_014394.1
1334; Asticcacaulis excentricus CB 48 chromosome 1, complete sequence;
315497051; NC_014816.1
1335; Burkholderia gladioli BSR3 chromosome 1, complete sequence;
327367349; CP002599.1
1336; Sphingobium chlorophenolicum L-1 chromosome 1, complete sequence;
334100279; CP002798.1
1337; Streptomyces violaceusniger Tu 4113, complete genome; 345007964;
NC_015957.1
1338; Rhodospirillum rubrum F11, complete genome; 386348020; NC_017584.1
1339; Actinoplanes sp. SE50/110, complete genome; 386845069; NC_017803.1
1340; Bacillus thuringiensis MC28, complete genome; 407703236; NC_018693.1
1341; Desulfocapsa sulfexigens DSM 10523, complete genome; 451945650;
NC_020304.1
1342; Xanthomonas citri pv. punicae str. LMG 859, whole genome shotgun
sequence; 390991205; NZ_CAGJ01000031.1
1343; Streptomyces fulvissimus DSM 40593, complete genome; 488607535;
NC_021177.1
1344; Streptomyces rapamycinicus NRRL 5491 genome; 521353217;
CP006567.1
1345; Kutzneria albida strain NRRL B-24060 contig305.1, whole genome shotgun
sequence; 662161093; NZ_JNYH01000515.1
1346; Mesorhizobium huakuii 7653R genome; 657121522; CP006581.1
1347; Mesorhizobium huakuii 7653R genome; 657121522; CP006581.1
1348; Burkholderia thailandensis E555 BTHE555 314, whole genome shotgun
sequence; 485035557; NZ_AECN01000315.1
1349; Sphingopyxis fiibergensis strain Kp5.2, complete genome; 749188513;
NZ_CP009122.1
1350; Sphingopyxis fiibergensis strain Kp5.2, complete genome; 749188513;
NZ_CP009122.1
1351; Streptomyces sp. ZJ306 hydroxylase, deacetylase, and hypothetical proteins
genes, complete cds; ikarugamycin gene cluster, complete sequence; and GCN5-
related N-acetyltransferase, hypothetical protein, aspamgine synthase,
transcriptional regulator, ABC transporter, hypothetical proteins, putative
membrane transport protein, putative acetyltransferase, cytochrome P450, putative
alpha-glucosidase, phosphoketolase, helix-turn-helix domain-containing protein,
membrane protein, NAD-dependent epimera; 746616581; KF954512.1
1352; Streptomyces albus strain DSM 41398, complete genome; 749658562;
NZ_CP010519.1
1353; Amycolatopsis lurida NRRL 2430, complete genome; 755908329;
CP007219.1
1354; Streptomyces lydicus A02, complete genome; 822214995;
NZ_CP007699.1
1355; Streptomyces lydicus A02, complete genome; 822214995;
NZ_CP007699.1
1356; Streptomyces xiamenensis strain 318, complete genome; 921170702;
NZ_CP009922.2
1357; Streptomyces sp. PBH53 genome; 852460626; CP011799.1
1358; Streptomyces sp. PBH53 genome; 852460626; CP011799.1
1359; Streptomyces sp. PBH53 genome; 852460626; CP011799.1
1360; Sphingopyxis sp. 113P3, complete genome; 924898949; NZ_CP009452.1
1361; Sphingopyxis sp. 113P3, complete genome; 924898949; NZ_CP009452.1
1362; Nostoc piscinale CENA21 genome; 930349143; CP012036.1
1363; Sphingopyxis macrogoltabida strain 203, complete genome; 938956730;
NZ_CP009429.1
1364; Sphingopyxis macrogoltabida strain 203 plasmid, complete sequence;
938956814;
1365; Paenibacillus sp. 320-W, complete genome; 961447255; CP013653.1
1366; Streptomyces avermitilis MA-4680 = NBRC 14893, complete genome;
162960844; NC_003155 .4
1367; Kitasatospora setae KM-6054 DNA, complete genome; 357386972;
NC_016109.1
1368; Rhodococcus jostii lariatin biosynthetic gene cluster (larA, larB, larC, larD, larE),
complete cds; 380356103; AB593691.1
1369; Rubrivivax gelatinosus IL144 DNA, complete genome; 383755859;
NC_017075.1
1370; Fischerellathermalis PCC 7521 contig00099, whole genome shotgun
sequence; 484076371; NZ_AJLL01000098.1
1371; Streptococcus suis 5C84 complete genome, strain 5C84; 253750923;
NC_012924.1
1372; Enterococcus faecalis ATCC 29212 contig24, whole genome shotgun
sequence; 401673929; ALOD01000024.1
1373; Roseburia sp. CAG:197 WGS project CBBL01000000 data, contig, whole
genome shotgun sequence; 524261006; CBBL010000225.1
1374; Closltidium sp. CAG:221 WGS project CBDC01000000 data, contig,
whole genome shotgun sequence; 524362382; CBDC010000065.1
1375; Closltidium sp. CAG:411 WGS project CBIY01000000 data, contig, whole
genome shotgun sequence; 524742306; CBIY010000075.1
1376; Novosphingobium sp. KN65.2 WGS project CCBH000000000 data, contig
SPHv1_Contig_228, whole genome shotgun sequence; 808402906;
CCBH010000144.1
1377; Mesorhizobium plurifarium genome assembly Mesorhizobium plurifarium
ORS1032T genome assembly, contig MPL1032_Contig_21, whole genome
shotgun sequence; 927916006; CCND01000014.1
1378; Kibdelosporangium sp. MJ126-NF4, whole genome shotgun sequence;
754819815; NZ_CDME01000002.1
1379; Methanobacterium formicicum genome assembly isolate Mb9,
chromosome: I; 952971377; LN734822.1
1380; Streptococcus pneumoniae strain 37, whole genome shotgun sequence;
912676034; NZ_CMPZ01000004.1
1381; Streptococcus pneumoniae strain type strain: N, whole genome shotgun
NZ_CP009430.1 sequence; 950938054; NZ_CIHL01000007.1
1382; Streptococcus pneumoniae strain 37, whole genome shotgun sequence;
912676034; NZ_CMPZ01000004.1
1383; Klebsiella variicola genome assembly Kv4880, contig BN1200_Contig_75,
whole genome shotgun sequence; 906292938; CXPB01000073.1
1384; Klebsiella variicola genome assembly KvT29A, contig
BN1200_Contig_98, whole genome shotgun sequence; 906304012;
CXPA01000125.1
1385; Bacillus cereus genome assembly Bacillus JRS4, contig contig000025,
whole genome shotgun sequence; 924092470; CYHM01000025.1
1386; Achromobacter sp. 27895TDY5663426 genome assembly, contig.
ERS372662SCcontig000003, whole genome shotgun sequence; 928675838;
CYTQ01000003.1
1387; Pedobacter sp. BAL39 1103467000492, whole genome shotgun sequence;
149277373; NZ_ABCM01000005.1
1388; Streptomyces sp. Mg1 supercont1.100, whole genome shotgun sequence;
254387191; NZ_D5570483.1
1389; Streptomyces sviceus ATCC 29083 chromosome, whole genome shotgun
sequence; 297196766; NZ_CM000951.1
1390; Streptomyces pristinaespiralis ATCC 25486 chromosome, whole genome
shotgun sequence; 297189896; NZ_CM000950.1
1391; Streptomyces roseosporus NRRL 15998 supercont3.1 genomic scaffold,
whole genome shotgun sequence; 221717172; DS999644.1
1392; Streptococcus vestibularis F0396 ctg1126932565723, whole genome
shotgun sequence; 311100538; AEKO01000007.1
1393; Ruminococcus albus 8 contig00035, whole genome shotgun sequence;
325680876; NZ_ADKM02000123.1
1394; Streptomyces sp. W007 contig00293, whole genome shotgun sequence;
365867746; NZ_AGSW01000272.1
1395; Burkholderia pseudomallei 1258a Contig0089, whole genome shotgun
sequence; 418540998; NZ_AHJB01000089.1
1396; Burkholderia pseudomallei 1258a Contig0089, whole genome shotgun
sequence; 418540998; NZ_AHJB01000089.1
1397; Rhodanobacter sp. 115 contig437, whole genome shotgun sequence;
389759651; NZ_AJXS01000437.1
1398; Rhodanobacter thiooxydans LCS2 contig057, whole genome shotgun
sequence; 389809081; NZ_AJXW01000057.1
1399; Burkholderia thailandensis MSMB43 Scaffold3, whole genome shotgun
sequence; 424903876; NZ_JH692063.1
1400; Streptomyces auratus AGR0001 Scaffold1_85, whole genome shotgun
sequence; 396995461; AJGV01000085.1
1401; Uncultured bacterium ACD_75C02634, whole genome shotgun sequence;
406886663; AMFJ01033303.1
1402; Amycolatopsis decaplanina DSM 44594 Contig0055, whole genome
shotgun sequence; 458848256; NZ_AOHO01000055.1
1403; Streptomyces mobaraensis NBRC 13819 = DSM 40847 contig024, whole
genome shotgun sequence; 458977979; NZ_AORZ01000024.1
1404; Burkholderia mallei GB8 horse 4 contig 394, whole genome shotgun
sequence; 67639376; NZ_AAO001000116.1
1405; Enterococcus faecalis EnGen0363 strain RMC5 acAqY-supercont1.4,
whole genome shotgun sequence; 502232520; NZ_KB944632.1
1406; Enterococcus faecalis EnGen0233 strain UAA1014 acvJV-
supercont1.10.C18, whole genome shotgun sequence; 487281881;
AIZW01000018.1
1407; Pandoraea sp. SD6-2 scaffold29, whole genome shotgun sequence;
505733815; NZ_KB944444.1
1408; Streptomyces aurantiacus JA 4570 Seq28, whole genome shotgun sequence;
514916412; NZ_AOPZ01000028.1
1409; Streptomyces aurantiacus JA 4570 Seq17, whole genome shotgun sequence;
514916021; NZ_AOPZ01000017.1
1410; Enterococcus faecalis LA3B-2 Scaffold22, whole genome shotgun
sequence; 522837181; NZ_KE352807.1
1411; Paenibacillus alvei A6-6i-x PAAL66ix_14, whole genome shotgun
sequence; 528200987; ATMS01000061.1
1412; Dehalobacter sp. UNSWDHB Contig_139, whole genome shotgun
sequence; 544905305; NZ_AUUR01000139.1
1413; Actinobaculum sp. oral taxon 183 str. F0552 Scaffold15, whole genome
shotgun sequence; 545327527; NZ_KE951412.1
1414; Actinobaculum sp. oral taxon 183 str. F0552 A_P1HMPREF0043-1.0_Cont1.1,
whole genome shotgun sequence; 541476958; AWSB01000006.1
1415; Propionibacterium acidifaciens F0233 ctg1127964738299, whole genome
shotgun sequence; 544249812; ACVN02000045.1
1416; Rubidibacter lacunae KORDI 51-2 KR5l_contig00121, whole genome
shotgun sequence; 550281965; NZ_ASSJ01000070.1
1417; Rothia aeria F0184 R aerigIMPREF0742-1.0_Cont136.4, whole genome
shotgun sequence; 551695014; AXZG01000035.1
1418; Candidatus Halobonum tyrrellensis G22 contig00002, whole genome
shotgun sequence; 557371823; NZ_ASGZ01000002.1
1419; Blastomonas sp. CACIA14H2 contig00049, whole genome shotgun
sequence; 563282524; AYSC01000019.1
1420; Frankia sp. CcI6 CcI6DRAFT_scaffold_51.52, whole genome shotgun
sequence; 563312125; AYTZ01000052.1
1421; Frankia sp. CeD CEDDRAFT_scaffold_22.23, whole genome shotgun
sequence; 737947180; NZ_JPGU01000023.1
1422; Closltidium butyricum DORA_1 Q607_CBUC00058, whole genome
shotgun sequence; 566226100; AZLX01000058.1
1423; Streptococcus sp. DORA_10 Q617_SPSC00257, whole genome shotgun
sequence; 566231608; AZMH01000257.1
1424; Candidatus Entotheonella gemina TSY2_contig00559, whole genome
shotgun sequence; 575423213; AZHX01000559.1
1425; Streptomyces roseosporus NRRL 15998 supercont3.1 genomic scaffold,
whole genome shotgun sequence; 221717172; DS999644.1
1426; Frankia sp. CcI6 CcI6DRAFT_scaffold_51.52, whole genome shotgun
sequence; 563312125; AYTZ01000052.1
1427; Frankia sp. Thr ThrDRAFT_scaffold_28.29, whole genome shotgun
sequence; 602262270; JENI01000029.1
1428; Novosphingobium resinovorum strain KF1 contig000008, whole genome
shotgun sequence; 738615271; NZ_JFYZ01000008.1
1429; Brevundimonas abyssalis TAR-001 DNA, contig: BAB005, whole genome
shotgun sequence; 543418148; BATC01000005.1
1430; Bacillus akibai JCM 9157, whole genome shotgun sequence; 737696658;
NZ_BAUV01000025.1
1431; Bacillus boroniphilus JCM 21738 DNA, contig: contig_6, whole genome
shotgun sequence; 571146044; BAUW01000006.1
1432; Gracilibacillus boraciitolerans JCM 21714 DNA, contig:contig_30, whole
genome shotgun sequence; 575082509; BAVS01000030.1
1433; Bacterium endosymbiont of Mortierella elongata FMR23-6, whole genome
shotgun sequence; 779889750; NZ_DF850521.1
1434; Sphingopyxis sp. C-1 DNA, contig contig_1, whole genome shotgun
sequence; 834156795; BBRO01000001.1
1435; Sphingopyxis sp. C-1 DNA, contig contig_1, whole genome shotgun
sequence; 834156795; BBRO01000001.1
1436; Ideonella sakaiensis strain 201-F6, whole genome shotgun sequence;
928998724; NZ_BBYR01000007.1
1437; Brevundimonas sp. EAKA contig5, whole genome shotgun sequence;
737322991; NZ_JMQR01000005.1
1438; Streptomyces griseorubens strain JSD-1 contig143, whole genome shotgun
sequence; 657284919; JJMG01000143.1
1439; Frankia sp. CeD CEDDRAFT_scaffold_22.23, whole genome shotgun
sequence; 737947180; NZ_JPGU01000023.1
1440; Frankia sp. CcI6 CcI6DRAFT_scaffold_51.52, whole genome shotgun
sequence; 563312125; AYTZ01000052.1
1441; Frankia sp. CeD CEDDRAFT_scaffold_22.23, whole genome shotgun
sequence; 737947180; NZ_JPGU01000023.1
1442; Bifidobacterium callitrichos DSM 23973 contig4, whole genome shotgun
sequence; 759443001; NZ_JDUV01000004.1
1443; Streptomyces sp. JS01 contig2, whole genome shotgun sequence;
695871554; NZ_JPWW01000002.1
1444; Sphingopyxis sp. LC81 contig43, whole genome shotgun sequence;
686469310; JNFD01000038.1
1445; Sphingopyxis sp. LC81 contig24, whole genome shotgun sequence;
739659070; NZ_JNFD01000017.1
1446; Sphingopyxis sp. LC363 contig36, whole genome shotgun sequence;
739702045; NZ_JNFC01000030.1
1447; Burkholderia pseudomallei strain BEF DP42.Contig323, whole genome
shotgun sequence; 686949962; JPNR01000131.1
1448; Xanthomonas cannabis pv. phaseoli strain Nyagatare scf 52938_7, whole
genome shotgun sequence; 835885587; NZ_KN265462.1
1449; Burkholderia pseudomallei M5HR435 Y033.Contig530, whole genome
shotgun sequence; 715120018; JRFP01000024.1
1450; Candidatus Thiomargarita nelsonii isolate Hydrate Ridge contig_1164,
whole genome shotgun sequence; 723288710; JSZA01001164.1
1451; Novosphingobium sp. P6W scaffold9, whole genome shotgun sequence;
763095630; NZ_JXZE01000009.1
1452; Streptomyces griseus strain S4-7 contig113, whole genome shotgun
sequence; 764464761; NZ_JYBE01000113.1
1453; Peptococcaceae bacterium BRH c4b BRHa_1001357, whole genome
shotgun sequence; 780813318; LAD001000010.1
1454; Streptomyces rubellomurinus subsp. indigoferus strain ATCC 31304 contig-
55, whole genome shotgun sequence; 783374270; NZ_JZKG01000056.1
1455; Streptomyces sp. NRRL S-444 c0ntig322.4, whole genome shotgun
sequence; 797049078; JZWX01001028.1
1456; Candidate division TM6 bacterium GW2011_GWF2_36_131
US03_C0013, whole genome shotgun sequence; 818310996; LBRK01000013.1
1457; Sphingobium czechense LL01 25410_1, whole genome shotgun sequence;
861972513; JACT01000001.1
1458; Streptomyces caatingaensis strain CMAA 1322 contig02, whole genome
shotgun sequence; 906344334; NZ_LFXA01000002.1
1459; Paenibacillus polymyxa strain YUPP-8 scaffold32, whole genome shotgun
sequence; 924434005; LIYK01000027.1
1460; Burkholderi amallei GB8 horse 4 contig_394, whole genome shotgun
sequence; 67639376; NZ_AAHO01000116.1
1461; Streptomyces rimosus subsp. rimosus ATCC 10970 contig00312, whole
genome shotgun sequence; 441176881; NZ_ANSJ01000243.1
1462; Streptomyces rimosus subsp. rimosus ATCC 10970 contig00333, whole
genome shotgun sequence; 441178796; NZ_ANSJ01000259.1
1463; Streptomyces rimosus subsp. rimosus ATCC 10970 contig00312, whole
genome shotgun sequence; 441176881; NZ_ANSJ01000243.1
1464; Streptomyces rimosus subsp. rimosus ATCC 10970 contig00333, whole
genome shotgun sequence; 441178796; NZ_ANSJ01000259.1
1465; Streptomyces rimosus subsp. rimosus ATCC 10970 contig00333, whole
genome shotgun sequence; 441178796; NZ_ANSJ01000259.1
1466; Streptomyces rimosus subsp. rimosus ATCC 10970 contig00333, whole
genome shotgun sequence; 441178796; NZ_ANSJ01000259.1
1467; Streptomyces rimosus subsp. rimosus strain NRRL WC-3924 contig82.1,
whole genome shotgun sequence; 663379797; NZ_JOBW01000082.1
1468; Streptomyces sp. NRRL F-5755 P309contig7.1, whole genome shotgun
sequence; 926371541; NZ_LGCW01000295.1
1469; Streptomyces sp. NRRL F-5755 P309contig48.1, whole genome shotgun
sequence; 926371517; NZ_LGCW01000271.1
1470; Streptomyces sp. NRRL F-6491 P443contig15.1, whole genome shotgun
sequence; 925610911; LGEE01000058.1
1471; Streptomyces sp. NRRL S-444 contig322.4, whole genome shotgun
sequence; 797049078; JZWX01001028.1
1472; Actinobacteria bacterium OK074 ctg60, whole genome shotgun sequence;
930473294; NZ_LJCV01000275.1
1473; Betaproteobacteria bacterium 5G8 39 WOR 8-12 2589, whole genome
shotgun sequence; 931421682; LJTQ01000030.1
1474; Candidate division BRC1 bacterium SM23_51 WORSMTZ_10094 whole
genome shotgun sequence; 931536013; LJUL01000022.1
1475; Bacillus vietnamensis strain UCD-SED5 scaffold 15, whole genome
shotgun sequence; 933903534; LIXZ01000017.1
1476; Xanthomonas arboricola strain CITA 44 CITA_44_contig_26, whole
genome shotgun sequence; 937505789; NZ_LJGM01000026.1
1477; Xanthomonas sp. Mitacek01 contig_17, whole genome shotgun sequence;
941965142; NZ_LKIT01000002.1
1478; Erythrobacteraceae bacterium HL-111 ITZY_scaf_51, whole genome
shotgun sequence; 938259025; LJSW01000006.1
1479; Halomonas sp. HL-93 ITZY_scaf_415, whole genome shotgun sequence;
938285459; LJST01000237.1
1480; Paenibacillus sp. Soi1724D2 contig_11, whole genome shotgun sequence;
946400391; LMRY01000003.1
1481; Streptomyces silvensis strain ATCC 53525 53525_Assembly_Contig_22,
whole genome shotgun sequence; 970361514; LOCL01000028.1
1482; Bacillus cereus R309803 chromosome, whole genome shotgun sequence;
238801472; NZ_CM000720.1
1483; Streptococcus pneumoniae strain P18082 isolate E3GXY, whole genome
shotgun sequence; 935445269; NZ_CIEC02000098.1
1484; Streptococcus pneumoniae strain 37, whole genome shotgun sequence;
912676034; NZ_CMPZ01000004.1
1485; Bacillus cereus Rock3-44 chromosome, whole genome shotgun sequence;
238801485; NZ_CM000733.1
1486; Bacillus cereus VDM006 acrHb-supercont1.1, whole genome shotgun
sequence; 507060269; NZ_KB976864.1
1487; Bacillus cereus AH1271 chromosome, whole genome shotgun sequence;
238801491; NZ_CM000739.1
1488; Bacillus cereus VD115 supercont1.1, whole genome shotgun sequence;
423614674; NZ_JH792165.1
1489; Bacillus thuringiensis MC28, complete genome; 407703236; NC_018693.1
1490; Bacillus thuringiensis serovar andalousiensis BGSC 4AW1 chromosome,
whole genome shotgun sequence; 238801506; NZ_CM000754.1
1491; Bacillus cereus BAG3X2-1 supercont1.1, whole genome shotgun sequence;
423416528; NZ_JH791923.1
1492; Escherichia coli strain EC2_3 Contig93, whole genome shotgun sequence;
742921760; NZ_JWKL01000093.1
1493; Bacillus cereus NVH0597-99 gcontig2_1106483384196, whole genome
shotgun sequence; 196038187; NZ_ABDK02000003.1
1494; Bacillus cereus VD142 actaa-supercont2.2, whole genome shotgun
sequence; 514340871; NZ_KE150045.1
1495; Bacillus cereus BAG5X2-1 supercont1.1, whole genome shotgun sequence;
423456860; NZ_JH791975.1
1496; Bacillus cereus BAG60-2 supercont1.1, whole genome shotgun sequence;
423468694; NZ_JH804628.1
1497; Bacillus cereus HuA2-9 acqVt-supercont1.1, whole genome shotgun
sequence; 507020427; NZ_KB976152.1
1498; Bacillus cereus HuA3-9 acqVv-supercont1.4, whole genome shotgun
sequence; 507024338; NZ_KB976146.1
1499; Bacillus cereus MC67 supercont1.2, whole genome shotgun sequence;
423557538; NZ_JH792114.1
1500; Bacillus cereus AH621 chromosome, whole genome shotgun sequence;
238801471; NZ_CM000719.1
1501; Bacillus cereus VD107 supercont1.1, whole genome shotgun sequence;
423609285; NZ_JH792232.1
1502; Bacillus cereus VDM034 supercont1.1, whole genome shotgun sequence;
423666303; NZ_JH791809.1
1503; Enterococcus faecalis D6 supercont1.4, whole genome shotgun sequence;
242358782; NZ_GG688629.1
1504; Enterococcus faecalis EnGen0363 strain RMC5 acAqY-supercont1.4,
whole genome shotgun sequence; 502232520; NZ_KB944632.1
1505; Enterococcus faecalis TX1341 Scfld578, whole genome shotgun sequence;
422736691; NZ_GL457197.1
1506; Rhodobacter sphaeroides WS8N chromosome chrI, whole genome shotgun
sequence; 332561612; NZ_CM001161.1
1507; Ruminococcus albus 8 contig00035, whole genome shotgun sequence;
325680876; NZ_ADKM02000123.1
1508; Brevundimonas diminuta ATCC 11568 BDIM_scaffold00005, whole
genome shotgun sequence; 329889017; NZ_GL883086.1
1509; Brevundimonas diminuta 470-4 Scfld7, whole genome shotgun sequence;
444405902; NZ_KB291784.1
1510; Clostridium butyricum 5521 gcontig_1106103650482, whole genome
shotgun sequence; 182420360; NZ_ABDT01000120.2
1511; Clostridium butyricum strain HM-68 Contig83, whole genome shotgun
sequence; 760273878; NZ_JXBT01000001.1
1512; Xanthomonas citti pv. punicae str. LMG 859, whole genome shotgun
sequence; 390991205; NZ_CAGJ01000031.1
1513; Streptomyces clavuligerus ATCC 27064 supercont1.55, whole genome
shotgun sequence; 254392242; NZ_DS570678.1
1514; Streptomyces rimosus subsp. rimosus ATCC 10970 contig00312, whole
genome shotgun sequence; 441176881; NZ_ANSJ01000243.1
1515; Streptomyces rimosus subsp. rimosus ATCC 10970 contig00333, whole
genome shotgun sequence; 441178796; NZ_ANSJ01000259.1
1516; Streptomyces viridochromogenes DSM 40736 supercont1.1, whole genome
shotgun sequence; 224581107; NZ_GG657757.1
1517; Streptomyces viridochromogenes DSM 40736 supercont1.1, whole genome
shotgun sequence; 224581107; NZ_GG657757.1
1518; Streptomyces viridochromogenes Tue57 Seq127, whole genome shotgun
sequence; 443625867; NZ_AMLP01000127.1
1519; Methanobacterium formicicum DSM 3637 Contig04, whole genome
shotgun sequence; 408381849; NZ_AMP001000004.1
1520; Burkholderia mallei GB8 horse 4 contig_394, whole genome shotgun
sequence; 67639376; NZ_AAH001000116.1
1521; Sphingobium yanoikuyae ATCC 51230 supercont1.1, whole genome
shotgun sequence; 427407324; NZ_JH992904.1
1522; Sphingobium yanoikuyae strain SHJ scaffold2, whole genome shotgun
sequence; 893711333; NZ_KQ235984.1
1523; Burkholderia mallei GB8 horse 4 contig_394, whole genome shotgun
sequence; 67639376; NZ_AAH001000116.1
1524; Burkholderia pseudomallei 1710b chromosome 1, complete sequence;
76808520; NC_007434.1
1525; Burkholderia pseudomallei 1258a Contig0089, whole genome shotgun
sequence; 418540998; NZ_AHJB01000089.1
1526; Burkholderia pseudomallei strain BEF DP42.Contig323, whole genome
shotgun sequence; 686949962; JPNR01000131.1
1527; [Eubacterium] cellulosolvens 6 chromosome, whole genome shotgun
sequence; 389575461; NZ_CM001487.1
1528; Streptomyces mobaraensis NBRC 13819 = DSM 40847 contig024, whole
genome shotgun sequence; 458977979; NZ_AORZ01000024.1
1529; Streptomyces mobaraensis NBRC 13819 = DSM 40847 contig079, whole
genome shotgun sequence; 458984960; NZ_AORZ01000079.1
1530; Amycolatopsis azurea DSM 43854 contig60, whole genome shotgun
sequence; 451338568; NZ_ANMG01000060.1
1531; Streptomyces pristinaespiralis ATCC 25486 chromosome, whole genome
shotgun sequence; 297189896; NZ_CM000950.1
1532; Xanthomonas axonopodis pv. malvacearum str. GSPB1386
1386_Scaffold6, whole genome shotgun sequence; 418516056; NZ_AHIB01000006.1
1533; Burkholderia thailandensis MSMB43 Scaffold3, whole genome shotgun
sequence; 424903876; NZ_JH692063.1
1534; Xanthomonas gardneri ATCC 19865 XANTHO7DRAF_Contig52, whole
genome shotgun sequence; 325923334; NZ_AEQX01000392.1
1535; Leptolyngbya sp. PCC 7375 Lepto7375DRAFT_LPA.5, whole genome
shotgun sequence; 427415532; NZ_JH993797.1
1536; Streptomyces auratus AGR0001 Scaffold1, whole genome shotgun
sequence; 398790069; NZ_JH725387.1
1537; Halosimplex carlsbadense 2-9-1 contig_4, whole genome shotgun sequence;
448406329; NZ_AOIU01000004.1
1538; Rothia aeria F0474 contig00003, whole genome shotgun sequence;
383809261; NZ_AJJQ01000036.1
1539; Sphingobium japonicum BiD32, whole genome shotgun sequence;
494022722; NZ_CAVK010000217.1
1540; Amycolatopsis decaplanina DSM 44594 Contig0055, whole genome
shotgun sequence; 458848256; NZ_AOH001000055.1
1541; Fictibacillus macauensis ZFHKF-1 Contig20, whole genome shotgun
sequence; 392955666; NZ_AKKV01000020.1
1542; Paenibacillus sp. Aloe-11 GW8_15, whole genome shotgun sequence;
375307420; NZ_JH601049.1
1543; Rhodanobacter denitrificans strain 116-2 contig032, whole genome shotgun
sequence; 389798210; NZ_AJXV01000032.1
1544; Caulobacter sp. AP07 PMI01_contig_53.53, whole genome shotgun
sequence; 399069941; NZ_AKKF01000033.1
1545; Novosphingobium sp. AP12 PMI02_contig_78.78, whole genome shotgun
sequence; 399058618; NZ_AKKE01000021.1
1546; Sphingobium sp. AP49 PMI04_contig490.490, whole genome shotgun
sequence; 398386476; NZ_AJVL01000086.1
1547; Moorea producens 3L scf52054, whole genome shotgun sequence;
332710503; NZ_GL890955.1
1548; Rhodanobacter sp. 115 contig437, whole genome shotgun sequence;
389759651; NZ_AJXS01000437.1
1549; Pedobacter sp. BAL39 1103467000500, whole genome shotgun sequence;
149277003; NZ_ABCM01000004.1
1550; Pedobacter sp. BAL39 1103467000492, whole genome shotgun sequence;
149277373; NZ_ABCM01000005.1
1551; Sulfurovum sp. AR contig00449, whole genome shotgun sequence;
386284588; NZ_AJLE01000006.1
1552; Mucilaginibacter paludis DSM 18603 chromosome, whole genome shotgun
sequence; 373951708; NZ_CM001403.1
1553; Magnetospirillum caucaseum strain SO-1 contig00006, whole genome
shotgun sequence; 458904467; NZ_AONQ01000006.1
1554; Streptomyces sp. Mg1 supercont1.100, whole genome shotgun sequence;
254387191; NZ_DS570483.1
1555; Sphingomonas sp. LH128 Contig3, whole genome shotgun sequence;
402821166; NZ_ALVC01000003.1
1556; Sphingomonas sp. LH128 Contig8, whole genome shotgun sequence;
402821307; NZ_ALVC01000008.1
1557; Streptomyces sp. AA4 supercont1.3, whole genome shotgun sequence;
224581098; NZ_GG657748.1
1558; Cecembia lonarensis LW9 contig000133, whole genome shotgun sequence;
406663945; NZ_AMGM01000133.1
1559; Actinomyces sp. oral taxon 848 str. F0332 Scfld0, whole genome shotgun
sequence; 260447107; NZ_GG703879.1
1560; Streptomyces ipomoeae 91-03 gcontig_1108499715961, whole genome
shotgun sequence; 429196334; NZ_AEJC01000180.1
1561; Frankia sp. QA3 chromosome, whole genome shotgun sequence;
392941286; NZ_CM001489.1
1562; Fischerella thermalis PCC 7521 contig00099, whole genome shotgun
sequence; 484076371; NZ_AJLL01000098.1
1563; Rhodobacter sp. AKP1 contig19, whole genome shotgun sequence;
429208285; NZ_ANFS01000019.1
1564; Rubrivivax benzoatilyticus JA2 = ATCC BAA-35 strain JA2 contig_155,
whole genome shotgun sequence; 332527785; NZ_AEWG01000155.1
1565; Burkholderia thailandensis E555 BTHE555_314, whole genome shotgun
sequence; 485035557; NZ_AECNO1000315.1
1566; Burkholdefia thailandensis E555 BTHE555 314, whole genome shotgun
sequence; 485035557; NZ_AECNO1000315.1
1567; Streptomyces chartreusis NRRL 12338 12338 Doro1_scaffold19, whole
genome shotgun sequence; 381200190; NZ_JH164855.1
1568; Streptomyces globisporus C-1027 Scaffold24_1, whole genome shotgun
sequence; 410651191; NZ_AJUO01000171.1
1569; Streptomyces roseosporus NRRL 15998 supercont3.1 genomic scaffold,
whole genome shotgun sequence; 221717172; DS999644.1
1570; Burkholdefia oklahomensis EO147 PMP6xxBPSxxE0147-248, whole
genome shotgun sequence; 149146238; NZ_ABBF01000248.1
1571; Burkholdefia oklahomensis C6786 PMP6xxBOKxxC6786-168, whole
genome shotgun sequence; 149147045; NZ_ABBG01000168.1
1572; Candidatus Odyssella thessalonicensis L13 HMO_scaffold00016, whole
genome shotgun sequence; 343957487; NZ_AEWF01000005.1
1573; Candidatus Odyssella thessalonicensis L13 HMO_scaffold00016, whole
genome shotgun sequence; 343957487; NZ_AEWF01000005.1
1574; Sphingobium yanoikuyae XLDN2-5 contig000022, whole genome shotgun
sequence; 378759068; NZ_AFXE01000022.1
1575; Sphingobium yanoikuyae XLDN2-5 contig000029, whole genome shotgun
sequence; 378759075; NZ_AFXE01000029.1
1576; Paenibacillus peofiae KCTC 3763 contig9, whole genome shotgun
sequence; 389822526; NZ_AGFX01000048.1
1577; Citromicrobium sp. JLT1363 contig00009, whole genome shotgun
sequence; 341575924; NZ_AEUE01000009.1
1578; Acaryochlofis sp. CCMEE 5410 contig00232, whole genome shotgun
sequence; 359367134; NZ__AFEJ01000154.1
1579; Stenotrophomonas maltophilia strain 419_SMAL
707_128228_1961615_4_642_523_, whole genome shotgun sequence;
896535166; NZ_JVHW01000017.1
1580; Streptomyces sp. S4, whole genome shotgun sequence; 358468601;
NZ_FR873700.1
1581; Pandoraea sp. 5D6-2 scaffold29, whole genome shotgun sequence;
505733815; NZ_KB944444.1
1582; Mesorhizobium loti MAFF303099 DNA, complete genome; 57165207;
NC_002678.2
1583; Streptomyces avermitilis MA-4680 = NBRC 14893, complete genome;
162960844; NC_003155 .4
1584; Thermobifida fusca TM51 contig028, whole genome shotgun sequence;
510814910; NZ_AOSG01000028.1
1585; Rhodobacter sphaeroides 2.4.1 chromosome 1, whole genome shotgun
sequence; 482849861; NZ_AKBU01000001.1
1586; Rhodospirillum rubrum F11, complete genome; 386348020; NC_017584.1
1587; Rhodospirillum rubrum F11, complete genome; 386348020; NC_017584.1
1588; Frankia sp. CcI6 CcI6DRAFT_scaffold_51.52, whole genome shotgun
sequence; 563312125; AYTZ01000052.1
1589; Roseobacter denitfificans OCh 114, complete genome; 110677421;
NC_008209.1
1590; Rhodobacter sphaeroides ATCC 17029 chromosome 1, complete sequence;
126460778; NC_009049.1
1591; Rhodobacter sphaeroides ATCC 17025, complete genome; 146276058;
NC_009428.1
1592; Streptococcus suis SC84 complete genome, strain SC84; 253750923;
NC_012924.1
1593; Geobacter uraniireducens Rf4, complete genome; 148262085;
NC_009483.1
1594; Sulfurovum sp. NBC37-1 genomic DNA, complete genome; 152991597;
NC_009663.1
1595; Acaryochloris marina MBIC11017, complete genome; 158333233;
NC_009925.1
1596; Bacillus weihenstephanensis KBAB4, complete genome; 163938013;
NC_010184.1
1597; Caulobacter sp. K31 plasmid pCAUL01, complete sequence; 167621728;
NC_010335.1
1598; Caulobacter sp. K31, complete genome; 167643973; NC_010338.1
1599; Candidatus Amoebophilus asiaticus 5a2, complete genome; 189501470;
NC_010830.1
1600; Stenotrophomonas maltophilia R551-3, complete genome; 194363778;
NC_011071.1
1601; Cyanothece sp. PCC 7425, complete genome; 220905643; NC_011884.1
1602; Chitinophaga pinensis DSM 2588, complete genome; 256419057;
NC_013132.1
1603; Haliangium ochraceum DSM 14365, complete genome; 262193326;
NC_013440.1
1604; Thermobaculum terrenum ATCC BAA-798 chromosome 2, complete
sequence; 269838913; NC_013526.1
1605; Xylanimonas cellulosilytica DSM 15894, complete genome; 269954810;
NC_013530.1
1606; Stackebrandtia nassauensis DSM 44728, complete genome; 291297538;
NC_013947.1
1607; Sphingobium japonicum UT26S DNA, chromosome 1, complete genome;
294009986;
1608; Sphingobium japonicum UT26S plasmid pCHQ1 DNA, complete genome;
294023656; NC_014007.1
1609; Butyrivibrio proteoclasticus B316 chromosome 1, complete sequence;
302669374; NC_014387.1
1610; Paenibacillus jamilae strain NS115 contig_27, whole genome shotgun
sequence; 970428876; NZ_LDRX01000027.1
1611; Frankia inefficax, complete genome; 312193897; NC_014666.1
1612; Asticcacaulis excentricus CB 48 chromosome 1, complete sequence;
315497051; NC_014816.1
1613; Teniglobus saanensis SP1PR4, complete genome; 320105246;
NC_014963.1
1614; Methanobacterium lacus strain AL-21, complete genome; 325957759;
NC_015216.1
1615; Marinomonas meditenanea MMB-1, complete genome; 326793322;
NC_015276.1
1616; Desulfobacca acetoxidans DSM 11109, complete genome; 328951746;
NC_015388.1
1617; Methanobacterium paludis strain SWAN1, complete genome; 333986242;
NC_015574.1
1618; Frankia symbiont of Datisca glomerata, complete genome; 336176139;
NC_015656.1
1619; Halopiger xanaduensis SH-6 plasmid pHALXA01, complete genome;
336251750; NC_015658.1
1620; Mesorhizobium opportunistum WSM2075, complete genome; 337264537;
NC_015675.1
1621; Runella slithyformis DSM 19594, complete genome; 338209545;
NC_015703.1
1622; Roseobacter litoralis Och 149, complete genome; 339501577;
NC_015730.1
1623; Streptomyces violaceusniger Tu 4113 plasmid pSTRVI01, complete
sequence; 345007457; NC_015951.1
1624; Streptomyces violaceusniger Tu 4113, complete genome; 345007964;
NC_015957.1
1625; Sphingobium sp. SYK-6 DNA, complete genome; 347526385;
NC_014006.1 NC_015976.1
1626; Chloracidobacterium thermophilum B chromosome 1, complete sequence;
347753732; NCO16024.1
1627; Kitasatospora setae KM-6054 DNA, complete genome; 357386972;
NC_016109.1
1628; Streptomyces cattleya str. NRRL 8057 main chromosome, complete
genome; 357397620; NC_016111.1
1629; Legionella pneumophila subsp. pneumophila ATCC 43290, complete
genome; 378775961; NC_016811.1
1630; Rubrivivax gelatinosus IL144 DNA, complete genome; 383755859;
NC_017075.1
1631; Francisella cf novicida 3523, complete genome; 387823583; NC_017449.1
1632; Rhodospirillum rubrum F11, complete genome; 386348020; NC_017584.1
1633; Actinoplanes sp. SE50/110, complete genome; 386845069; NC_017803.1
1634; Legionella pneumophila subsp. pneumophila str. Lonaine chromosome,
complete genome; 397662556; NC_018139.1
1635; Emticicia oligotrophica DSM 17448, complete genome; 408671769;
NC_018748.1
1636; Streptomyces venezuelae ATCC 10712 complete genome; 408675720;
NC_018750.1
1637; Nostoc sp. PCC 7107, complete genome; 427705465; NC_019676.1
1638; Nostoc sp. PCC 7524, complete genome; 427727289; NC_019684.1
1639; Crinalium epipsammum PCC 9333, complete genome; 428303693;
NC_019753.1
1640; Thermobacillus composti KWC4, complete genome; 430748349;
NC_019897.1
1641; Mesorhizobium australicum WSM2073, complete genome; 433771415;
NC_019973.1
1642; Desulfocapsa sulfexigens DSM 10523, complete genome; 451945650;
NC_020304.1
1643; Rhodanobacter denitrificans strain 2APBS1, complete genome; 469816339;
NC_020541.1
1644; Burkholderia thailandensis MSMB121 chromosome 1, complete sequence;
488601775;
1645; Streptomyces fulvissimus DSM 40593, complete genome; 488607535;
NC_021177.1
1646; Streptomyces davawensis strain JCM 4913 complete genome; 471319476;
NC_020504.1
1647; Streptomyces davawensis strain JCM 4913 complete genome; 471319476;
NC_020504.1
1648; Desulfotomaculum acetoxidans DSM 771, complete genome; 258513366;
NC_013216.1
1649; Desulfotomaculum acetoxidans DSM 771, complete genome; 258513366;
NC_013216.1
1650; Actinosynnema mirum DSM43827, complete genome; 256374160;
NC_013093.1
1651; Bacillus cereus BAG20-3 acfXF-supercont1.1, whole genome shotgun
sequence; 507017505; NZ_KB976530.1
1652; Bacillus cereus VD118 acrHo-supercont1.9, whole genome shotgun
sequence; 507035131; NZ_KB976800.1
1653; Bacillus cereus VDM053 acrGS-supercont1.7, whole genome shotgun
sequence; 507060152; NZ_KB976714.1
1654; Halomonas anticariensis FP35 = DSM 16096 strain FP35 Scaffold', whole
genome shotgun sequence; 514429123; NZ_KE332377.1
1655; Halomonas anticariensis FP35 = DSM 16096 strain FP35 Scaffold', whole
genome shotgun sequence; 514429123; NZ_KE332377.1
1656; Streptomyces sp. NRRL F-5639 contig75.1, whole genome shotgun
sequence; 664515060; NZ_JOGK01000075.1
1657; Acinetobacter gyllenbergii MTCC 11365 contigl, whole genome shotgun
sequence; 514348304; NZ_ASQH01000001.1
1658; Streptomyces aurantiacus JA 4570 Seq17, whole genome shotgun sequence;
514916021; NZ_AOPZ01000017.1
1659; Streptomyces aurantiacus JA 4570 Seq28, whole genome shotgun sequence;
514916412; NZ_AOPZ01000028.1
1660; Streptomyces aurantiacus JA 4570 Seq63, whole genome shotgun sequence;
514917321; NZ_AOPZ01000063.1
1661; Streptomyces aurantiacus JA 4570 Seq109, whole genome shotgun
sequence; 514918665; NZ_AOPZ01000109.1
1662; Paenibacillus polymyxa OSY-DF Contig136, whole genome shotgun
NC_021173.1 sequence; 484036841; NZ_AIPP01000136.1
1663; Fischerella muscicola SAG 1427-1 = PCC 73103 contig00215, whole
genome shotgun sequence; 484073367; NZ_AJLJ01000207.1
1664; Fischerella muscicola PCC 7414 contig00153, whole genome shotgun
sequence; 484075372; NZ_AJLK01000153.1
1665; Xanthomonas arboricola pv. corylina str. NCCB 100457 Contig50, whole
genome shotgun sequence; 507418017; NZ_APMCO2000050.1
1666; Sphingobium xenophagum QYY contig015, whole genome shotgun
sequence; 484272664; NZ_AKM01000015.1
1667; Pedobacter arcticus A12 Scaffold2, whole genome shotgun sequence;
484345004; NZ_JH947126.1
1668; Leptolyngbya boryana PCC 6306 LepboDRAFT_LPC.1, whole genome
shotgun sequence; 482909028; N_KB731324.1
1669; Fischerella sp. PCC 9339 PCC9339DRAFT_scaffold1.1, whole genome
shotgun sequence; 482909394; NZJI-1992898.1
1670; Mastigocladopsis repens PCC 10914 Mas10914DRAFT_scaffold1.1, whole
genome shotgun sequence; 482909462; NZ_J14992901.1
1671; Lactococcus garvieae Tac2 Tac2Contig_33, whole genome shotgun
sequence; 483258918; NZ_AMFE01000033.1
1672; Paenisporosarcina sp. TG-14 111.TG14.1_1, whole genome shotgun
sequence; 483299154; NZ_AMGD01000001.1
1673; Amphibacillus jilinensis Y1 Scaffold2, whole genome shotgun sequence;
483992405; NZ_JH976435.1
1674; Alpha proteobacterium LLX12A LLX12A_contig00014, whole genome
shotgun sequence; 483996931; NZ_AMYX01000014.1
1675; Alpha proteobacterium LLX12A LLX12A_contig00026, whole genome
shotgun sequence; 483996974; NZ_AMYX01000026.1
1676; Alpha proteobacterium LLX12A LLX12A_contig00084, whole genome
shotgun sequence; 483997176; NZ_AMYX01000084.1
1677; Alpha proteobacterium L4 1A L41A_contig00002, whole genome shotgun
sequence; 483997957; NZ_AMYY01000002.1
1678; Nocardiopsis alba DSM 43377 contig_34, whole genome shotgun
sequence; 484007204; NZ_ANAC01000034.1
1679; Nocardiopsis halophila DSM 44494 contig_138, whole genome shotgun
sequence; 484007841; NZ_ANAD01000138.1
1680; Nocardiopsis halophila DSM 44494 contig_197, whole genome shotgun
sequence; 484008051; NZ_ANAD01000197.1
1681; Nocardiopsis halotolerans DSM 44410 contig_372, whole genome shotgun
sequence; 484016556; NZ_ANAX01000372.1
1682; Nocardiopsis lucentensis DSM 44048 contig_935, whole genome shotgun
sequence; 484021665; NZ_ANBC01000935.1
1683; Nocardiopsis alkaliphila YIM 80379 contig_111, whole genome shotgun
sequence; 484022237; NZ_ANBD01000111.1
1684; Nocardiopsis chromatogenes YIM 90109 contig_93, whole genome
shotgun sequence; 484026206; NZ_ANBH01000093.1
1685; Porphyrobacter sp. AAP82 Contig35, whole genome shotgun sequence;
484033307; NZ_ANFX01000035.1
1686; Blastomonas sp. AAP53 Contig8, whole genome shotgun sequence;
484033611; NZ_ANFZ01000008.1
1687; Blastomonas sp. AAP53 Contig14, whole genome shotgun sequence;
484033631; NZ_ANFZ01000014.1
1688; Paenibacillus sp. PAMC 26794 5104_29, whole genome shotgun sequence;
484070054; NZ_ANHX01000029.1
1689; Oscillatoria sp. PCC 10802 Osc10802DRAFT_Contig7.7, whole genome
shotgun sequence; 484104632; NZ_KB235948.1
1690; Clostridium botulinum CB11/1-1 CB contig00105, whole genome shotgun
sequence; 484141779; NZ_AORM01000006.1
1691; Actinopolyspora halophila DSM 43834 ActhaDRAFT_contig1.1_C, whole
genome shotgun sequence; 484203522; NZ_AQUI01000002.1
1692; Asticcacaulis benevestitus DSM 16100 =ATCC BAA-896 strain DSM
16100 B060DRAFT_scaffold_12.13_C, whole genome shotgun sequence;
484226753; NZ_AQWM01000013.1
1693; Asticcacaulis benevestitus DSM 16100 = ATCC BAA-896 strain DSM
16100 B060DRAFT_scaffold_31.32S, whole genome
484226810; NZ_AQWM01000032.1
1694; Streptomyces sp. FxanaC1 B074DRAFT_scaffold_1.2_C, whole genome
shotgun sequence; 484227180; NZ_AQW001000002.1
1695; Streptomyces sp. FxanaC1 B074DRAFT_scaffold_7.8_C, whole genome
shotgun sequence; 484227195; NZ_AQW001000008.1
1696; Smamgdicoccus niigatensis DSM 44881 =NBRC 103563 strain DSM
44881 F600DRAFT_scaffold00011.11_C, whole genome shotgun sequence;
484234624; NZ_AQXZ01000009.1
1697; Verrucomicrobium sp. 3C A37ADRAFT_scaffold1.1, whole genome
shotgun sequence; 483219562; NZ_KB901875.1
1698; Verrucomicrobium sp. 3C A37ADRAFT_scaffold1.1, whole genome
shotgun sequence; 483219562; NZ_KB901875.1
1699; Bradyrhizobium sp. WSM2793 A3ASDRAFT_scaffold_24.25, whole
genome shotgun sequence; 483314733; NZ_KB902785.1
1700; Streptomyces vitaminophilus DSM 41686 A3IGDRAFT_scaffold_10.11,
whole genome shotgun sequence; 483682977; NZ_KB904636.1
1701; Streptomyces sp. CcalMP-8W B053DRAFT_scaffold_17.18, whole
genome shotgun sequence; 483961830; NZ_KB890924.1
1702; Streptomyces sp. ScaeMP-e10 B061DRAFT scaffold_01, whole genome
shotgun sequence; 483967534; NZ_KB891296.1
1703; Streptomyces sp. KhCrAH-244 B069DRAFT_scaffold_11.12, whole
genome shotgun sequence; 483969755; NZ_KB891596.1
1704; Streptomyces sp. HmicA12 B072DRAFT_scaffold_19.20, whole genome
shotgun sequence; 483972948; NZ_KB891808.1
1705; Streptomyces sp. MspMP-M5 B073DRAFT_scaffold 27.28, whole
genome shotgun sequence; 483974021; NZ_KB891893.1
1706; Bacillus mycoides strain Flugge 10206 DJ94.contig-100_16, whole genome
shotgun sequence; 727343482; NZ_JMQD01000030.1
1707; Streptomyces sp. CNY228 D330DRAFT_scaffold00011.11, whole genome
shotgun sequence; 484057944; NZ_KB898231.1
1708; Streptomyces sp. CNB091 D581DRAFT_scaffold00010.10, whole genome
shotgun sequence; 484070161; NZ_KB898999.1
1709; Sphingobium xenophagum NBRC 107872, whole genome shotgun
sequence; 483527356; NZ_BARE01000016.1
1710; Sphingobium xenophagum NBRC 107872, whole genome shotgun
shotgun sequence; sequence; 483532492; NZ_BARE01000100.1
1711; Bacillus oceanisediminis 2691 contig2644, whole genome shotgun
sequence; 485048843; NZ_ALEG01000067.1
1712; Bacillus sp. REN51N contig_2, whole genome shotgun sequence;
748816024; NZ_JXAB01000002.1
1713; Calothrix sp. PCC 7103 Cal7103DRAFT_CPM.6, whole genome shotgun
sequence; 485067373; NZ_KB217478.1
1714; Pseudanabaena sp. PCC 6802 Pse6802_scaffold_5, whole genome shotgun
sequence; 485067426; NZ_KB235914.1
1715; Actinopolyspora mortivallis DSM 44261 strain HS-1
ActmoDRAFT_scaffold1.1, whole genome shotgun sequence; 486324513;
NZ_KB913024.1
1716; Mesorhizobium huakuii 7653R genome; 657121522; CP006581.1
1717; Paenibacillus sp. FIW567 B212DRAFT_scaffold1.1, whole genome
shotgun sequence; 486346141; NZ_KB910518.1
1718; Bacillus sp. 123MFChir2 H280DRAFT scaffold00030.30, whole genome
shotgun sequence; 487368297; NZ_KB910953.1
1719; Streptomyces canus 299MFChir4.1 H293DRAFT_scaffold00032.32, whole
genome shotgun sequence; 487385965; NZ_KB911613.1
1720; Kribbella catacumbae DSM 19601 A3ESDRAFT scaffold_7.8_C, whole
genome shotgun sequence; 484207511; NZ_AQUZ01000008.1
1721; Paenibacillus riograndensis SBR5 Contig78, whole genome shotgun
sequence; 485470216; NZ__A
1722; Nonomumea coxensis DSM 45129 A3G7DRAFT_scaffold_4.5, whole
genome shotgun sequence; 483454700; NZ_KB903974.1
1723; Spirosoma spitsbergense DSM 19989 B157DRAFT_scaffold_76.77, whole
genome shotgun sequence; 483994857; NZ_KB893599.1
1724; Amycolatopsis alba DSM 44262 scaffold1, whole genome shotgun
sequence; 486330103; NZ_KB913032.1
1725; Amycolatopsis nigrescens CSC17Ta-90 AmyniDRAFT_Contig68.1_C,
whole genome shotgun sequence; 487404592; NZ_ARVW01000001.1
1726; Reyranella massiliensis 521, whole genome shotgun sequence; 484038067;
NZ_HE997181.1
1727; Acidobacteriaceae bacterium KBS 83 G002DRAFT_scaffold00007.7,
whole genome shotgun sequence; 485076323; NZ_KB906739.1
1728; Novosphingobium lindaniclasticum LE124 contig147, whole genome
shotgun sequence; 544819688; NZ_ATHL01000147.1
1729; Actinobaculum sp. oral taxon 183 str. F0552 A_P1HMPREF0043-
1.0_Cont1.1, whole genome shotgun sequence; 541476958; AWSB01000006.1
1730; Sphingomonas-like bacterium B12, whole genome shotgun sequence;
484113405; NZ_BACX01000237.1
1731; Sphingomonas-like bacterium B12, whole genome shotgun sequence;
484113491; NZ_BACX01000258.1
1732; Thermoactinomyces vulgaris strain NRRL F-5595 F5595contig15.1, whole
genome shotgun sequence; 929862756; NZ_LGKI01000090.1
1733; Closltidium saccharobutylicum DSM 13864, complete genome;
550916528; NC_022571.1
1734; Butyrivibrio fibrisolvens AB2020 G616DRAFT_scaffold00015.15_C,
whole genome shotgun sequence; 551012921; NZ_ATVZ01000015.1
1735; Butyrivibrio sp. XPD2006 G590DRAFT_scaffold00008.8_C, whole
genome shotgun sequence; 551021553; NZ_ATVT01000008.1
1736; Butyrivibrio sp. AE3009 G588DRAFT_scaffold00030.30_C, whole
genome shotgun sequence; 551035505; NZ_ATVS01000030.1
1737; Acidobacteriaceae bacterium TAA166 strain TAA 166
H979DRAFT_scaffold_0.1S, whole genome shotgun sequence; 551216990;
NZ_ATWD01000001.1
1738; Rothia aeria F0184 R_aeriaHMPREF0742-1.0_Cont136.4, whole genome
shotgun sequence; 551695014; AXZG01000035.1
1739; Klebsiella pneumoniae 4541-2 4541_2_67, whole genome shotgun
sequence; 657698352; NZ_JDW001000067.1
1740; Klebsiella pneumoniae MGH 19 addTc-supercont1.2, whole genome
shotgun sequence; 556494858; NZ_KI535678.1
1741; Candidatus Halobonum tyrrellensis G22 contig00002, whole genome
shotgun sequence; 557371823; NZ_ASGZ01000002.1
1742; Asticcacaulis sp. AC466 contig00008, whole genome shotgun sequence;
557833377; NZ_AWGE01000008.1
1743; Asticcacaulis sp. AC466 contig00033, whole genome shotgun sequence;
557835508; NZ_AWGE01000033.1
1744; Asticcacaulis sp. YBE204 contig00005, whole genome shotgun sequence;
557839256; NZ_AWGF01000005.1
1745; Asticcacaulis sp. YBE204 contig00010, whole genome shotgun sequence;
557839714; NZ_AWGF01000010.1
1746; Streptomyces roseochromogenus subsp. oscitans DS 12.976 chromosome,
whole genome shotgun sequence; 566155502; NZ_CM002285.1
1747; Bacillus boroniphilus JCM 21738 DNA, contig: contig_6, whole genome
shotgun sequence; 571146044; BAUW01000006.1
1748; Mesorhizobium sp. LNHC232B00 scaffold0020, whole genome shotgun
sequence; 563561985; NZ_AYWP01000020.1
1749; Mesorhizobium sp. LNHC220B00 scaffold0002, whole genome shotgun
sequence; 563576979; NZ_AYWS01000002.1
1750; Mesorhizobium sp. LNHC221B00 scaffold0001, whole genome shotgun
sequence; 563570867; NZ_AYWR01000001.1
1751; Clostridium pasteurianum NRRL B-598, complete genome; 930593557;
NZ_CP011966.1
1752; Paenibacillus peoriae strain HS311, complete genome; 922052336;
NZ_CP011512.1
1753; Magnetospirillum gryphiswaldense MSR-1 v2, complete genome;
568144401; NC_023065.1
1754; Streptococcus suis strain LS8F, whole genome shotgun sequence;
766589647; NZ_CEHJ01000007.1
1755; Bradyrhizobium sp. ARR65 BraARR65DRAFT_scaffold_9.10_C, whole
genome shotgun sequence; 639168743; NZ_AWZU01000010.1
1756; Paenibacillus sp. MAEPY2 contig7, whole genome shotgun sequence;
639451286; NZ_AWUK01000007.1
1757; Verrucomicrobia bacterium LP2A G346DRAFT_scf7180000000012_quiver.2_C,
whole genome shotgun sequence; 640169055; NZ_JAFS01000002.1
1758; Verrucomicrobia bacterium LP2A G346DRAFT_scf7180000000012_quiver.2_C,
whole genome shotgun sequence; 640169055; NZ_JAFS01000002.1
1759; Robbsia andropogonis Ba3549 160, whole genome shotgun sequence;
640451877; NZ_AYSW01000160.1
1760; Xanthomonas arboricola 3004 contig00003, whole genome shotgun
sequence; 640500871; NZ_AZQY01000003.1
1761; Bacillus mannanilyticus JCM 10596, whole genome shotgun sequence;
640600411; NZ_BAMO01000071.1
1762; Bacillus sp. H1a Contig_1, whole genome shotgun sequence; 640724079;
NZ_AYMH01000001.1
1763; Enterococcus faecalis ATCC 4200 supercont1.2, whole genome shotgun
sequence; 239948580; NZ_GG670372.1
1764; Haloglycomyces albus DSM 45210 HalalDRAFT chromosome 1.1_C,
whole genome shotgun sequence; 644043488; NZ_AZUQ01000001.1
1765; Sphingomonas sanxanigenens NX02, complete genome; 749321911;
NZ_CP006644.1
1766; Kutzneria albida strain NRRL B-24060 contig305.1, whole genome shotgun
sequence; 662161093; NZ_JNYH01000515.1
1767; Kutzneria albida DSM 43870, complete genome; 754862786;
NZ_CP007155.1
1768; Paenibacillus sp. ICGEB2008 Contig_7, whole genome shotgun sequence;
483624383; NZ_AMQUO1000007.1
1769; Sphingobium barthaii strain KK22, whole genome shotgun sequence;
646529442; NZ_BATN01000092.1
1770; Paenibacillus polymyxa 1-43 S143_contig00221, whole genome shotgun
sequence; 647225094; NZ_ASRZ01000173.1
1771; Paenibacillus graminis RSA19 S2_contig00597, whole genome shotgun
sequence; 647256651; NZ_ASSG01000304.1
1772; Paenibacillus polymyxa TD94 STD94_contig00759, whole genome
shotgun sequence; 647274605; NZ_ASSA01000134.1
1773; Bacillus flexus T6186-2 contig_106, whole genome shotgun sequence;
647636934; NZ_JANV01000106.1
1774; Brevundimonas naejangsanensis strain B1 contig000018, whole genome
shotgun sequence; 647728918; NZ_JHOF01000018.1
1775; Sphingomonas-like bacterium B12, whole genome shotgun sequence;
484115568; NZ_BACX01000797.1
1776; Nocardiopsis potens DSM 45234 contig_25, whole genome shotgun
sequence; 484017897; NZ_ANBB01000025.1
1777; Nocardiopsis halotolerans DSM 44410 contig_26, whole genome shotgun
sequence; 484015294; NZ_ANAX01000026.1
1778; Nocardiopsis baichengensis YIM 90130 Scaffold15_1, whole genome
shotgun sequence; 484012558; NZ_ANAS01000033.1
1779; Nocardiopsis alba DSM 43377 contig_10, whole genome shotgun
sequence; 484007121; NZ_ANAC01000010.1
1780; Sphingomonas melonis DAPP-PG 224 Sphme3DRAFT_scaffold1.1, whole
genome shotgun sequence; 482984722; NZ_KB900605.1
1781; Acidobacteriaceae bacterium TAA166 strain TAA 166
H979DRAFT_scaffold_0.1S, whole genome shotgun sequence; 551216990;
NZ_ATWD01000001.1
1782; Actinomadura oligospora ATCC 43269 P696DRAFT_scaffold00008.8_C,
whole genome shotgun sequence; 651281457; NZ_JADG01000010.1
1783; Butyrivibrio sp. XPD2002 G587DRAFT scaffold00011.11, whole genome
shotgun sequence; 651381584; NZ_KE384117.1
1784; Bacillus sp. UNC437CL72CviS29 M014DRAFT_scaffold00009.9_C,
whole genome shotgun sequence; 651596980; NZ_AXVB01000011.1
1785; Butyrivibrio sp. FC2001 G601DRAFT_scaffold00001.1, whole genome
shotgun sequence; 651921804; NZ_KE384132.1
1786; Bacillus bogoriensis ATCC BAA-922 T323DRAFT_scaffold00008.8_C,
whole genome shotgun sequence; 651937013; NZ_JHYI01000013.1
1787; Fischerella sp. PCC 9431 Fis9431DRAFT_Scaffold1.2, whole genome
shotgun sequence; 652326780; NZ_KE650771.1
1788; Fischerella sp. PCC 9605 FIS9605DRAFT_scaffold2.2, whole genome
shotgun sequence; 652337551; NZ_KI912149.1
1789; Clostridium akagii DSM 12554 BR66DRAFT_scaffold00010.10_C, whole
genome shotgun sequence; 652488076; NZ_JMLK01000014.1
1790; Glomeribacter sp. 1016415 H174DRAFT scaffold00001.1, whole genome
shotgun sequence; 652527059; NZ_KE384226.1
1791; Mesorhizobium sp. URHA0056 H959DRAFT_scaffold00004.4_C, whole
genome shotgun sequence; 652670206; NZ_AUEL01000005.1
1792; Mesorhizobium sp. URHA0056 H959DRAFT_scaffold00004.4_C, whole
genome shotgun sequence; 652670206; NZ_AUEL01000005.1
1793; Mesorhizobium loti R88b Meslo2DRAFT_Scaffold1.1, whole genome
shotgun sequence; 652688269; NZ_KI912159.1
1794; Mesorhizobium loti R88b Meslo2DRAFT_Scaffold1.1, whole genome
shotgun sequence; 652688269; NZ_KI912159.1
1795; Mesorhizobium ciceri W5M4083 MESCI2DRAFT_scaffold_01, whole
genome shotgun sequence; 652698054; NZ_K1912610.1
1796; Mesorhizobium sp. URHC0008 N549DRAFT_scaffold00001.1_C, whole
genome shotgun sequence; 652699616; NZ_JIAP01000001.1
1797; Mesorhizobium huakuii 7653R genome; 657121522; CP006581.1
1798; Mesorhizobium erdmanii USDA 3471 A3AUDRAFT_scaffold_7.8_C,
whole genome shotgun sequence; 652719874; NZ_AXAE01000013.1
1799; Mesorhizobium erdmanii USDA 3471 A3AUDRAFT_scaffold_7.8S,
whole genome shotgun sequence; 652719874; NZ_AXAE01000013.1
1800; Mesorhizobium loti CJ3sym A3A9DRAFT_scaffold_25.26_C, whole
genome shotgun sequence; 652734503; NZ_AXAL01000027.1
1801; Cobnella thennotolerans DSM 17683 G485DRAFT_scaffold00003.3,
whole genome shotgun sequence; 652794305; NZ_KE386956.1
1802; Mesorhizobium sp. WSM3626 Mesw3626DRAFT_scaffold_6.7_C, whole
genome shotgun sequence; 652879634; NZ_AZUY01000007.1
1803; Mesorhizobium sp. W5M1293 MesloDRAFT_scaffold_4.5, whole genome
shotgun sequence; 652910347; NZ_KI911320.1
1804; Legionella pneumophila subsp. pneumophila strain ATCC 33155
contig032, whole genome shotgun sequence; 652971687; NZ_JFIN01000032.1
1805; Legionella pneumophila subsp. pneumophila strain ATCC 33154 Scaffold2,
whole genome shotgun sequence; 653016013; NZ_KK074241.1
1806; Legionella pneumophila subsp. pneumophila strain ATCC 33823 Scaffold7,
whole genome shotgun sequence; 653016661; NZ_KK074199.1
1807; Bacillus sp. URHB0009 H980DRAFT_scaffold00016.16_C, whole
genome shotgun sequence; 653070042; NZ_AUER01000022.1
1808; Lachnospira multipara MC2003 T520DRAFT_scaffold00007.7_C, whole
genome shotgun sequence; 653225243; NZ_RIWY01000011.1
1809; Rhodanobacter sp. OR87 RhoOR87DRAFT_scaffold_24.25S, whole
genome shotgun sequence; 653308965; NZ_AXBJ01000026.1
1810; Rhodanobacter sp. OR92 RhoOR92DRAFT scaffold_6.7_C, whole
genome shotgun sequence; 653321547; NZ_ATYFO1000013.1
1811; Rhodanobacter sp. OR444 RHOOR444DRAFT
NODE_5_len_27336_cov_289_843719.5_C, whole
genome shotgun sequence; 653325317; NZ_ATYD01000005.1
1812; Rhodanobacter sp. OR444 RHOOR444DRAFT
NODE_39_len_52063_cov_320_872864.39, whole
genome shotgun sequence; 653330442; NZ_KE386531.1
1813; Bradyrhizobium sp. Aila-2 K288DRAFT_scaffold00086.86_C, whole
genome shotgun sequence; 653556699; NZ_AUEZ01000087.1
1814; Streptomyces sp. CNH099 B121DRAFT_scaffold_16.17_C, whole
genome shotgun sequence; 654239557; NZ_AZWL01000018.1
1815; Mastigocoleus testarum BC008 Contig-2, whole genome shotgun sequence;
959926096, NZ_LMTZ01000085.1
1816; [Eubacterium] cellulosolvens LD2006 T358DRAFT_scaffold00002.2_C,
whole genome shotgun sequence; 654392970; NZ_JHXY01000005.1
1817; Caulobacter sp. URHA0033 H963DRAFT_scaffold00023.23_C, whole
genome shotgun sequence; 654573246; NZ_AUE001000025.1
1818; Legionella pneumophila subsp. fraseri strain ATCC 35251 contig031, whole
genome shotgun sequence; 654928151; NZ_JFIG01000031.1
1819; Bacillus sp. FJAT-14578 Scaffold2, whole genome shotgun sequence;
654948246; NZ_K1632505.1
1820; Bacillus sp. 278922_107 H622DRAFT_scaffold00001.1, whole genome
shotgun sequence; 654964612; NZ_KI911354.1
1821; Streptomyces sp. SolWspMP-sol2th B083DRAFT_scaffold_17.18_C,
whole genome shotgun sequence; 654969845; NZ_ARPF01000020.1
1822; Ruminococcus flavefaciens ATCC 19208 L870DRAFT_scaffold00001.1,
whole genome shotgun sequence; 655069822; NZ_KI912489.1
1823; Paenibacillus sp. UNCCL52 BRO1DRAFT_scaffold00001.1, whole
genome shotgun sequence; 655095448; NZ_KK366023.1
1824; Paenibacillus taiwanensis DSM 18679 H509DRAFT_scaffold00010.10_C,
whole genome shotgun sequence; 655095554; NZ_AULE01000001.1
1825; Paenibacillus sp. UNC451MF BP97DRAFT_scaffold00018.18_C, whole
genome shotgun sequence; 655103160; NZ_JMLS01000021.1
1826; Desulfobulbus japonicus DSM 18378 G493DRAFT_scaffold00011.11_C,
whole genome shotgun sequence; 655133038; NZ_AUCV01000014.1
1827; Novosphingobium sp. B-7 scaffold147, whole genome shotgun sequence;
514419386; NZ_KE148338.1
1828; Streptomyces flavidovirens DSM 40150 G412DRAFT_scaffold00009.9,
whole genome shotgun sequence; 655416831; NZ_KE386846.1
1829; Terasakiella pusilla DSM 6293 Q397DRAFT_scaffold00039.39_C, whole
genome shotgun sequence; 655499373; NZ_JHY001000039.1
1830; Pseudoxanthomonas suwonensis J43 Psesu2DRAFT_scaffold_44.45S,
whole genome shotgun sequence; 655566937; NZ_JAES01000046.1
1831; Salinatimonas rosea DSM 21201 G407DRAFT_scaffold00021.21_C,
whole genome shotgun sequence; 655990125; NZ_AUBC01000024.1
1832; Paenibacillus harenae DSM 16969 H581DRAFT_scaffold00004.4, whole
genome shotgun sequence; 656245934; NZ_KE383845.1
1833; Paenibacillus alginolyticus DSM 5050 = NBRC 15375 strain DSM 5050
G519DRAFT_scaffold00043.43_C, whole genome shotgun sequence;
656249802; NZ_AUGY01000047.1
1834; Bacillus sp. RP1137 contig_18, whole genome shotgun sequence;
657210762; NZ_AXZS01000018.1
1835; Streptomyces leeuwenhoekii strain C34(2013) c34_sequence_0501, whole
genome shotgun sequence; 657301257; NZ_AZSD01000480.1
1836; Brevundimonas bacteroides DSM 4726 Q333DRAFT_scaffold00004.4_C,
whole genome shotgun sequence; 657605746; NZ_JNIX01000010.1
1837; Bacillus thuringiensis LM1212 scaffold 08, whole genome shotgun
sequence; 657629081; NZ_AYPV01000024.1
1838; Lachnoclosltidium phytofermentans KNHs212
BO10DRAFT_scf7180000000004_quiver.1_C, whole genome shotgun sequence;
657706549; NZ_JNLM01000001.1
1839; Paenibacillus polymyxa strain NRRL B-30509 contig00003, whole genome
shotgun sequence; 766607514; NZ_JTH001000003.1
1840; Paenibacillus polymyxa strain WLY78 S6_contig00095, whole genome
shotgun sequence; 657719467; NZ_ALJV01000094.1
1841; Stenotrophomonas maltophilia RR-10 STMALcontig40, whole genome
shotgun sequence; 484978121; NZ_AGRB01000040.1
1842; [Scytonema hofmanni] UTEX 2349 To19009DRAFT TPD.8, whole
genome shotgun sequence; 657935980; NZ_KK073768.1
1843; Caulobacter sp. UNC358MFTsu5.1 BR39DRAFT_scaffold00002.2_C,
whole genome shotgun sequence; 659864921; NZ_JONW01000006.1
1844; Sphingomonas sp. UNC305MFCo15.2 BR78DRAFT scaffold00001.1_C,
whole genome shotgun sequence; 659889283; NZ_JOOE01000001.1
1845; Streptomyces monomycini strain NRRL B-24309 P063_Doro1_scaffold135,
whole genome shotgun sequence; 662059070; NZ_KL571162.1
1846; Streptomyces peruviensis strain NRRL ISP-5592 P18 l_Doro l_scaffold152,
whole genome shotgun sequence; 662097244; NZ_KL575165.1
1847; Streptomyces natalensis strain NRRL B-5314 P055_Doro1_scaffold13,
whole genome shotgun sequence; 662108422; NZ_KL570019.1
1848; Streptomyces natalensis ATCC 27448 Scaffold_33, whole genome shotgun
sequence; 764439507; NZ_JRKI01000027.1
1849; Streptomyces baamensis strain NRRL B-2842 P144_Doro1_scaffold6,
whole genome shotgun sequence; 662129456; NZ_KL573544.1
1850; Streptomyces decoyicus strain NRRL ISP-5087 P056_Doro1_scaffold78,
whole genome shotgun sequence; 662133033; NZ_KL570321.1
1851; Streptomyces baamensis strain NRRL B-2842 P144_Doro1_scaffold26,
whole genome shotgun sequence; 662135579; NZ_KL573564.1
1852; Streptomyces puniceus strain NRRL ISP-5083 contig3.1, whole genome
shotgun sequence; 663149970; NZ_JOBQ01000003.1
1853; Spirillospora albida strain NRRL B-3350 contig1.1, whole genome shotgun
sequence; 663122276; NZ_JOFJ01000001.1
1854; Streptomyces sp. NRRL S-481 P269_Doro1_scaffold20, whole genome
shotgun sequence; 664428976; NZ_KL585179.1
1855; Streptomyces sp. NRRL S-87 contig69.1, whole genome shotgun sequence;
663169513; NZ_JO
1856; Streptomyces katrae strain NRRL B-16271 contig33.1, whole genome
shotgun sequence; 663300513; NZ_JNZY01000033.1
1857; Streptomyces katrae strain NRRL B-16271 contig37.1, whole genome
shotgun sequence; 663300941; NZ_JNZY01000037.1
1858; Streptomyces sp. NRRL B-3229 contig5.1, whole genome shotgun
sequence; 663316931; NZ_JOGP01000005.1
1859; Streptomyces griseus subsp. griseus strain NRRL F-2227 contig41.1, whole
genome shotgun sequence; 664325626; NZ_JOIT01000041.1
1860; Streptomyces roseoverticillatus strain NRRL B-3500 contig22.1, whole
genome shotgun sequence; 663372343; NZ_JOFL01000022.1
1861; Streptomyces roseoverticillatus strain NRRL B-3500 contig43.1, whole
genome shotgun sequence; 663373497; NZ_JOFL01000043.1
1862; Streptomyces rimosus subsp. rimosus strain NRRL WC-3924 contig19.1,
whole genome shotgun sequence; 663376433; NZ_JOBW01000019.1
1863; Streptomyces rimosus subsp. rimosus strain NRRL WC-3924 contig82.1,
whole genome shotgun sequence; 663379797; NZ_JOBW01000082.1
1864; Streptomyces sp. NRRL F-5917 contig68.1, whole genome shotgun
sequence; 663414324; NZ_JOHQ01000068.1
1865; Streptomyces sp. NRRL S-1448 contig134.1, whole genome shotgun
sequence; 663421576; NZ_JOGE01000134.1
1866; Allokutzneria albata strain NRRL B-24461 contig22.1, whole genome
shotgun sequence; 663596322; NZ_JOEF01000022.1
1867; Sphingobium sp. DC-2 ODE 45, whole genome shotgun sequence;
663818579; NZ_JNAC01000042.1
1868; Streptomyces aureocirculatus strain NRRL ISP-5386 contig11.1, whole
genome shotgun sequence; 664013282; NZ_JOAP01000011.1
1869; Streptomyces cyaneofuscatus strain NRRL B-2570 contig9.1, whole
genome shotgun sequence; 664021017; NZ_JOEM01000009.1
1870; Streptomyces aureocirculatus strain NRRL ISP-5386 contig49.1, whole
genome shotgun sequence; 664026629; NZ_JOAP01000049.1
1871; Streptomyces sclerotialus strain NRRL B-2317 contig7.1, whole genome
shotgun sequence; 664034500; NZ_JODX01000007.1
1872; Streptomyces anulatus strain NRRL B-2873 contig21.1, whole genome
shotgun sequence; 664049400; NZ_JOEZ01000021.1
1873; Streptomyces globisporus subsp. globisporus strain NRRL B-2709
contig24.1, whole genome shotgun sequence; 664051798; NZ_JNZK01000024.1
1874; Streptomyces rimosus subsp. rimosus strain NRRL B-2660 contig14.1,
whole genome shotgun sequence; 664052786; NZ_JOES01000014.1
1875; Streptomyces rimosus subsp. rimosus strain NRRL B-2660 contig59.1,
whole genome shotgun sequence; 664061406; NZ_JOES01000059.1
1876; Streptomyces achromogenes subsp. achromogenes strain NRRL B-2120
contig2.1, whole genome shotgun sequence; 664063830; NZ_JODT01000002.1
1877; Streptomyces rimosus subsp. rimosus strain NRRL B-2660 contig124.1,
whole genome shotgun sequence; 664066234; NZ_JOES01000124.1
1878; Streptomyces albus subsp. albus strain NRRL B-2445 contig28.1, whole
genome shotgun sequence; 664095100; NZ_JOED01000028.1
1879; Streptomyces rimosus subsp. rimosus strain NRRL WC-3929 contig5.1,
whole genome shotgun sequence; 664104387; NZ_JOJJ01000005.1
1880; Streptomyces rimosus subsp. rimosus strain NRRL WC-3904 contig10.1,
whole genome shotgun sequence; 664126885; NZ_JOCQ01000010.1
1881; Streptomyces rimosus subsp. rimosus strain NRRL WC-3904 contig106.1,
whole genome shotgun sequence; 664141810; NZ_JOCQ01000106.1
1882; Streptomyces griseus subsp. griseus strain NRRL F-5144 contig19.1, whole
genome shotgun sequence; 664184565; NZ_JOGA01000019.1
1883; Streptomyces sp. NRRL F-2295 P395contig79.1, whole genome shotgun
sequence; 926288193; NZ_LGCY01000146.1
1884; Streptomyces xiamenensis strain 318, complete genome; 921170702;
NZ_CP009922.2
1885; Streptomyces griseus subsp. griseus strain NRRL F-5618 contig4.1, whole
genome shotgun sequence; 664233412; NZ_JOGN01000004.1
1886; Streptomyces lavenduligriseus strain NRRL ISP-5487 contig2.1, whole
genome shotgun sequence; 664244706; NZ_JOBD01000002.1
1887; Streptomyces lavenduligriseus strain NRRL ISP-5487 contig2.1, whole
genome shotgun sequence; 664244706; NZ_JOBD01000002.1
1888; Streptomyces sp. NRRL S-920 contig3.1, whole genome shotgun sequence;
664245663; NZ_JODF01000003.1
1889; Streptomyces sp. NRRL S-337 contig41.1, whole genome shotgun
sequence; 664277815; NZ_JOIX01000041.1
1890; Streptomyces griseus strain S4-7 contig113, whole genome shotgun
sequence; 764464761; NZ_JYBE01000113.1
1891; Streptomyces sp. NRRL F-4474 contig32.1, whole genome shotgun
sequence; 664323078; NZ_JOIB01000032.1
1892; Streptomyces sp. NRRL S-475 contig32.1, whole genome shotgun
sequence; 664325162; NZ_JOJB01000032.1
1893; Streptomyces sp. NRRL S-646 contig23.1, whole genome shotgun
sequence; 664421883; NZ_JODC01000023.1
1894; Streptomyces sp. NRRL S-1813 contig13.1, whole genome shotgun
sequence; 664466568; NZ_JOHB01000013.1
1895; Streptomyces sp. NRRL WC-3773 contig2.1, whole genome shotgun
sequence; 664478668; NZ_JOJI01000002.1
1896; Streptomyces sp. NRRL WC-3773 contig36.1, whole genome shotgun
sequence; 664487325; NZ_JOJI01000036.1
1897; Streptomyces olivaceus strain NRRL B-3009 contig20.1, whole genome
shotgun sequence; 664523889; NZ_JOFH01000020.1
1898; Streptomyces ochraceiscleroticus strain NRRL ISP-5594 contig9.1, whole
genome shotgun sequence; 664540649; NZ_JOAX01000009.1
1899; Streptomyces sp. NRRL S-118 P205 Doro1_scaffold2, whole genome
shotgun sequence; 664556736; NZ_KL591003.1
1900; Streptomyces sp. NRRL S-118 P205_Doro1_scaffold34, whole genome
shotgun sequence; 664565137; NZ_KL591029.1
1901; Streptomyces olindensis strain DAUFPE 5622 103, whole genome shotgun
sequence; 739918964; NZ_JJOH01000097.1
1902; Streptomyces sp. NRRL S-623 contig14.1, whole genome shotgun
sequence; 665522165; NZ_JOJC01000016.1
1903; Streptomyces durhamensis strain NRRL B-3309 contig3.1, whole genome
shotgun sequence; 665586974; NZ_JNXR01000003.1
1904; Streptomyces durhamensis strain NRRL B-3309 contig23.1, whole genome
shotgun sequence; 665604093; NZ_JNXR01000023.1
1905; Streptomyces roseochromogenus subsp. oscitans DS 12.976 chromosome,
whole genome shotgun sequence; 566155502; NZ_CM002285.1
1906; Leptolyngbya sp. Heron Island J 50, whole genome shotgun sequence;
553739852; NZ_AWNH01000066.1
1907; Leptolyngbya sp. Heron Island J 50, whole genome shotgun sequence;
553739852; NZ_AWNH01000066.1
1908; Sphingobium lactosutens DS20 contig107, whole genome shotgun
sequence; 544811486; NZ_ATDP01000107.1
1909; Streptomyces sp. NRRL F-5123 contig24.1, whole genome shotgun
sequence; 671535174; NZ_JOHY01000024.1
1910; Bacillus sp. MB2021 T349DRAFT_scaffold00010.10_C, whole genome
shotgun sequence; 671553628; NZ_JN1101000011.1
1911; Lachnospira multipara LB2003 T537DRAFT_scaffold00010.10_C, whole
genome shotgun sequence; 671578517; NZ_JNKW01000011.1
1912; Closltidium drakei strain SL1 contig_20, whole genome shotgun sequence;
692121046; NZ_JIBU02000020.1
1913; Candidatus Paracaedibacter symbiosus strain PRA9 Scaffold_1, whole
genome shotgun sequence; 692233141; NZ_JQAK01000001.1
1914; Stenotrophomonas maltophilia strain 53 contig_2, whole genome shotgun
sequence; 692316574; NZ_JRJA01000002.1
1915; Klebsiella variicola genome assembly Kv4880, contig BN1200_Contig_75,
whole genome shotgun sequence; 906292938; CXPB01000073.1
1916; Streptomyces alboviridis strain NRRL B-1579 contig18.1, whole genome
shotgun sequence; 695845602; NZ_JNWU01000018.1
1917; Streptomyces sp. CN5654 CD02DRAFT_scaffold00023.23S, whole
genome shotgun sequence; 695856316; NZ_JNLT01000024.1
1918; Streptomyces albus subsp. albus strain NRRL B-16041 contig26.1, whole
genome shotgun sequence; 695869320; NZ_JNWW01000026.1
1919; Streptomyces sp. JS01 contig2, whole genome shotgun sequence;
695871554; NZ_JPWW01000002.1
1920; Mesorhizobium ciceri CMG6 MescicDRAFT_scaffold_1.2_C, whole
genome shotgun sequence; 639162053; NZ_AWZS01000002.1
1921; Mesorhizobium japonicum R7A MesloDRAFT_Scaffold1.1, whole
genome shotgun sequence; 696358903; NZ_KI632510.1
1922; Stenotrophomonas maltophilia RA8, whole genome shotgun sequence;
493412056; NZ_CALM01000701.1
1923; Streptomyces griseus subsp. griseus strain NRRL B-2307 contig15.1, whole
genome shotgun sequence; 702684649; NZ_iNZI01000015.1
1924; Kitasatospora setae KM-6054 DNA, complete genome; 357386972;
NC_016109.1
1925; Streptomyces lydicus strain NRRL ISP-5461 contig41.1, whole genome
shotgun sequence; 702808005; NZ_JNZA01000041.1
1926; Streptomyces iakyrus strain NRRL ISP-5482 contig6.1, whole genome
shotgun sequence; 702914619; NZ_JNXI01000006.1
1927; Kibdelosporangium afidum subsp. largum strain NRRL B-24462
contig91.4, whole genome shotgun sequence; 703243970; NZ_JNYM01001429.1
1928; Streptomyces galbus strain KCCM 41354 contig00021, whole genome
shotgun sequence; 716912366; NZ_JRHJ01000016.1
1929; Bacillus aryabhattai strain GZ03 contig1_scaffoldl, whole genome shotgun
sequence; 723602665; NZ_JPIE01000001.1
1930; Bacillus mycoides FSL H7-687 Contig052, whole genome shotgun
sequence; 727271768; NZ_ASPY01000052.1
1931; Bacillus weihenstephanensis strain JAS 83/3 Bw_JAS-83/3_contig00005,
whole genome shotgun sequence; 910095435; NZ_JNLY01000005.1
1932; Sphingomonas sp. ERGS Contig80, whole genome shotgun sequence;
734983422; NZ_JSXI01000079.1
1933; Lachnospira multipara ATCC 19207 G600DRAFT_scaffold00009.9_C,
whole genome shotgun sequence; 653218978; NZ_AUJG01000009.1
1934; Bacillus sp. 72 T409DRAFT_scf7180000000077_quiver.15S, whole
genome shotgun sequence; 736160933; NZ_JQMI01000015.1
1935; Bacillus simplex BA2H3 scaffold2, whole genome shotgun sequence;
736214556; NZ_KN360955.1
1936; Dehalobacter sp. UNSWDHB Contig_139, whole genome shotgun
sequence; 544905305; NZ_AUUR01000139.1
1937; Actinomadura oligospora ATCC 43269 P696DRAFT_scaffold00008.8_C,
whole genome shotgun sequence; 651281457; NZ_JADG01000010.1
1938; Hyphomonas oceanitis 5CH89 contig59, whole genome shotgun sequence;
737569369; NZ_ARYL01000059.1
1939; Bacillus vietnamensis strain HD-02, whole genome shotgun sequence;
736762362; NZ_CCDN010000009.1
1940; Hyphomonas sp. CY54-11-8 contig4, whole genome shotgun sequence;
736764136; NZ_AWFD01000033.1
1941; Erythrobacter longus strain DSM 6997 contig9, whole genome shotgun
sequence; 736965849; NZ_JMIW01000009.1
1942; Caulobacter henricii strain CF287 EW90DRAFT_scaffold00023.23_C,
whole genome shotgun sequence; 737089868; NZ_JQJNO1000025.1
1943; Caulobacter henricii strain YR570 EX13DRAFT_scaffold00022.22_C,
whole genome shotgun sequence; 737103862; NZ_JQJP01000023.1
1944; Calothfix sp. 336/3, complete genome; 821032128; NZ_CP011382.1
1945; Bacillus firmus DS1 scaffold33, whole genome shotgun sequence;
737350949; NZ_APVL01000034.1
1946; Bacillus hemicellulosilyticus JCM 9152, whole genome shotgun sequence;
737360192; NZ_BAUU01000008.1
1947; Edaphobacter aggregans DSM 19364 Q363DRAFT_scaffold00032.32_C,
whole genome shotgun sequence; 737370143; NZ_JQKI01000040.1
1948; Bacillus sp. UNC322MFChir4.1 BR72DRAFT_scaffold00004.4, whole
genome shotgun sequence; 737456981; NZ_KN050811.1
1949; Hyphomonas oceanitis SCH89 contig20, whole genome shotgun sequence;
737567115; NZ_ARYL01000020.1
1950; Hyphomonas oceanitis SCH89 contig59, whole genome shotgun sequence;
737569369; NZ_ARYL01000059.1
1951; Halobacillus sp. BBL2006 cont444, whole genome shotgun sequence;
737576092; NZ_JRNX01000441.1
1952; Hyphomonas atlantica strain 22II1-22F38 contig10, whole genome shotgun
sequence; 737577234; NZ_AWFH01000002.1
1953; Hyphomonas atlantica strain 22II1-22F38 contig28, whole genome shotgun
sequence; 737580759; NZ_AWFH01000021.1
1954; Hyphomonas jannaschiana VP2 contig2, whole genome shotgun sequence;
737608363; NZ_ARYJ01000002.1
1955; Bacillus akibai JCM 9157, whole genome shotgun sequence; 737696658;
NZ_BAUV01000025.1
1956; Frankia sp. CeD CEDDRAFT_scaffold_22.23, whole genome shotgun
sequence; 737947180; NZ_JPGU01000023.1
1957; Clostridium butyricum strain NEC8, whole genome shotgun sequence;
960334134; NZ_CBYK010000003.1
1958; Clostridium butyricum AGR2140 G607DRAFT_scaffold00008.8_C,
whole genome shotgun sequence; 653632769; NZ_AUJN01000009.1
1959; Fusobacterium necrophorum BFTR-2 contig0075, whole genome shotgun
sequence; 737951550; NZ_JAAG01000075.1
1960; [Leptolyngbya] sp. JSC-1 Osccy1DRAFT_CYJSC_l_DRAF_scaffold00069.1,
whole genome shotgun sequence; 738050739; NZ_KL662191.1
1961; Bradyrhizobium sp. WSM1743 YU9DRAFT_scaffold_1.2_C, whole
genome shotgun sequence; 653526890; NZ_AXAZ01000002.1
1962; Mesorhizobium sp. WSM3224 YU3DRAFT_scaffold_3.4_C, whole
genome shotgun sequence; 652912253; NZ_ATY001000004.1
1963; Myxosarcina sp. GI1 contig_5, whole genome shotgun sequence;
738529722; NZ_JRFE01000006.1
1964; Novosphingobium resinovorum strain KF1 contig000002, whole genome
shotgun sequence; 738613868; NZ_JFYZ01000002.1
1965; Paenibacillus sp. FSL H7-689 Contig015, whole genome shotgun sequence;
738716739; NZ_ASPU01000015.1
1966; Paenibacillus wynnii strain DSM 18334 unitig_2, whole genome shotgun
sequence; 738760618; NZ_JQCR01000002.1
1967; Paenibacillus sp. FSL R7-269 Contig022, whole genome shotgun sequence;
738803633; NZ_ASPS01000022.1
1968; Paenibacillus pinihumi DSM 23905 = JCM 16419 strain DSM 23905
H583DRAFT_scaffold00005.5, whole genome shotgun sequence; 655115689;
NZ_KE383867.1
1969; Paenibacillus harenae DSM 16969 H58 1DRAFT_scaffold00002.2, whole
genome shotgun sequence; 655165706; NZ_KE383843.1
1970; Paenibacillus sp. FSL R7-277 Contig088, whole genome shotgun sequence;
738841140; NZ_ASPX01000088.1
1971; Pseudonocardia acaciae DSM 45401 N912DRAFT_scaffold00002.2_C,
whole genome shotgun sequence; 655569633; NZ_JIAI01000002.1
1972; Amycolatopsis orientalis DSM 40040 = KCTC 9412 contig_32, whole
genome shotgun sequence; 499136900; NZ_ASJB01000015.1
1973; Sphingobium chlorophenolicum strain NBRC 16172 contig000025, whole
genome shotgun sequence; 739594477; NZ_JFHR01000025.1
1974; Sphingobium herbicidovorans NBRC 16415 contig000028, whole genome
shotgun sequence; 739610197; NZ_JFZA02000028.1
1975; Sphingobium sp. bal seq0028, whole genome shotgun sequence;
739622900; NZ_JPPQ01000069.1
1976; Sphingomonas paucimobilis strain EPA505 contig000016, whole genome
shotgun sequence; 739629085; NZ_JFYY01000016.1
1977; Sphingomonas paucimobilis strain EPA505 contig000027, whole genome
shotgun sequence; 739630357; NZ_JFYY01000027.1
1978; Sphingobium yanoikuyae ATCC 51230 supercont1.1, whole genome
shotgun sequence; 427407324; NZ_JH992904.1
1979; Sphingobium yanoikuyae strain B1 scaffold28, whole genome shotgun
sequence; 739656825; NZ_KL662220.1
1980; Sphingobium yanoikuyae strain B1 contig000002, whole genome shotgun
sequence; 739661773; NZ_JGVR01000002.1
1981; Sphingomonas wittichii strain YR128 EX04DRAFT_scaffold00050.50_C,
whole genome shotgun sequence; 739674258; NZ_JQMC01000050.1
1982; Sphingomonas sp. SKA58 scf_1100007010440, whole genome shotgun
sequence; 211594417; NZ_CH959308.1
1983; Sphingopyxis sp. LC363 contig1, whole genome shotgun sequence;
739699072; NZ_JNFC01000001.1
1984; Sphingopyxis sp. LC363 contig30, whole genome shotgun sequence;
739701660; NZ_JNFC01000024.1
1985; Sphingopyxis sp. LC363 contig5, whole genome shotgun sequence;
739702995; NZ_JNFC01000045.1
1986; Streptococcus salivarius strain NU10 contig_11, whole genome shotgun
sequence; 739748927; NZ_BMT01000011.1
1987; Streptomyces griseoluteus strain NRRL ISP-5360 contig43.1, whole
genome shotgun sequence; 663180071; NZ_JOBE01000043.1
1988; Streptomyces griseorubens strain JSD-1 contig143, whole genome shotgun
sequence; 657284919; BMG01000143.1
1989; Streptomyces avermitilis MA-4680 = NBRC 14893, complete genome;
162960844; NC_003155.4
1990; Streptomyces achromogenes subsp. achromogenes strain NRRL B-2120
contig2.1, whole genome shotgun sequence; 664063830; NZ_JODT01000002.1
1991; Streptomyces griseus subsp. griseus strain NRRL WC-3645 contig40.1,
whole genome shotgun sequence; 739830264; NZ_JOJE01000040.1
1992; Streptomyces scabiei strain NCPPB 4086 scf 65433_365.1, whole genome
shotgun sequence; 739854483; NZ_KL997447.1
1993; Streptomyces sp. FXJ7.023 Contig10, whole genome shotgun sequence;
510871397; NZ_APIV01000010.1
1994; Streptomyces sp. PRh5 contig001, whole genome shotgun sequence;
740097110; NZ_JABQ01000001.1
1995; Paenibacillus sp. FSL H7-0357, complete genome; 749299172;
NZ_CP009241.1
1996; Paenibacillus stellifer strain DSM 14472, complete genome; 753871514;
NZ_CP009286.1
1997; Burkholderia pseudomallei strain MSHR4018 scaffold2, whole genome
shotgun sequence; 740942724; NZ_KN323080.1
1998; Burkholderia sp. ABCPW 111 X946.contig-100_0, whole genome shotgun
sequence; 740958729; NZ_JPWT01000001.1
1999; Cupriavidus sp. IDO NODE 7, whole genome shotgun sequence;
742878908; NZ_JWMA01000006.1
2000; Paenibacillus polymyxa strain DSM 365 Contig001, whole genome shotgun
sequence; 746220937; NZ_JMIQ01000001.1
2001; Paenibacillus polymyxa strain CF05 genome; 746228615; NZ_CP009909.1
2002; Novosphingobium malaysiense strain MUSC 273 Contig9, whole genome
shotgun sequence; 746241774; NZ_JIDI01000009.1
2003; Paenibacillus sp. IL-IB B 3415 contig_069, whole genome shotgun sequence;
746258261; NZ_JUB01000069.1
2004; Novosphingobium subtenaneum strain DSM 12447 NJ75_contig000013,
whole genome shotgun sequence; 746288194; NZ_JRVC01000013.1
2005; Pandoraea sputorum strain DSM 21091, complete genome; 749204399;
NZ_CP010431.1
2006; Xanthomonas cannabis pv. cannabis strain NCPPB 3753 contig_67, whole
genome shotgun sequence; 746366822; NZ_JSZF01000067.1
2007; Xanthomonas arboricola pv. pruni MAFF 301420 strain MAFF301420,
whole genome shotgun sequence; 759376814; NZ_BAVC01000017.1
2008; Xanthomonas arboricola pv. celebensis strain NCPPB 1630
scf_49108_10.1, whole genome shotgun sequence; 746486416; NZ_KL638873.1
2009; Xanthomonas arboricola pv. celebensis strain NCPPB 1832
scf_23466_141.1, whole genome shotgun sequence; 746494072;
NZ_KL638866.1
2010; Xanthomonas cannabis pv. cannabis strain NCPPB 2877 contig_94, whole
genome shotgun sequence; 746532813; NZ_JSZE01000094.1
2011; Sphingopyxis fiibergensis strain Kp5.2, complete genome; 749188513;
NZ_CP009122.1
2012; Sphingopyxis fiibergensis strain Kp5.2, complete genome; 749188513;
NZ_CP009122.1
2013; Streptomyces sp. 769, complete genome; 749181963; NZ_CP003987.1
2014; Hassallia byssoidea VB512170 scaffold_0, whole genome shotgun
sequence; 748181452; NZ_JTCM01000043.1
2015; Jeotgalibacillus malaysiensis strain D5 chromosome, complete genome;
749182744; NZ_CP009416.1
2016; Paenibacillus sp. FSL R7-0273, complete genome; 749302091;
NZ_CP009283.1
2017; Paenibacillus polymyxa strain Sb3-1, complete genome; 749204146;
NZ_CP010268.1
2018; Klebsiella pneumoniae CCHB01000016, whole genome shotgun sequence;
749639368; NZ_CCHB01000016.1
2019; Streptomyces albus strain DSM 41398, complete genome; 749658562;
NZ_CP010519.1
2020; Streptomonospora alba strain YIM 90003 contig_9, whole genome shotgun
sequence; 749673329; NZ_JR0001000009.1
2021; Uncultured marine bacterium 463 clone EBAC080-L32B05 genomic
sequence; 41582259; AY458641.2
2022; Nocardiopsis chromatogenes YIM 90109 contig_59, whole genome
shotgun sequence; 484026076; NZ_ANBH01000059.1
2023; Paenibacillus dendritiformis C454 PDENDC1000064, whole genome
shotgun sequence; 374605177; NZ_AHKH01000064.1
2024; Streptomyces auratus AGR0001 Scaffold1_85, whole genome shotgun
sequence; 396995461; AJGV01000085.1
2025; Tolypothrix campylonemoides VB511288 scaffold 0, whole genome
shotgun sequence; 751565075; NZ_JXCB01000004.1
2026; Jeotgalibacillus soli strain P9 c0ntig00009, whole genome shotgun
sequence; 751619763; NZ_JXRP01000009.1
2027; Cylindrospermum stagnale PCC 7417, complete genome; 434402184;
NC_019757.1
2028; Sphingopyxis alaskensis RB2256, complete genome; 103485498;
NC_008048.1
2029; Syntrophobotulus glycolicus DSM 8271, complete genome; 325288201;
NC_015172.1
2030; Novosphingobium aromaticivorans DSM 12444, complete genome;
87198026; NC_007794.1
2031; Novosphingobium sp. PP1Y Lpl large plasmid, complete replicon;
334133217; NC_015579.1
2032; Bacillus sp. 1NLA3E, complete genome; 488570484; NC_021171.1
2033; Burkholderia rhizoxinica HKI 454, complete genome; 312794749;
NC_014722.1
2034; Psychromonas ingrahamii 37, complete genome; 119943794; NC_008709.1
2035; Streptococcus salivarius JI1V18777 complete genome; 387783149;
NC_017595.1
2036; Actinosynnema mirum DSM 43827, complete genome; 256374160; whole
NC_013093.1
2037; Legionella pneumophila 2300/99 Alcoy, complete genome; 296105497;
NC_014125.1
2038; Paenibacillus sp. FSL R5-0912, complete genome; 754884871;
NZ_CP009282.1
2039; Streptomyces sp. NBRC 110027, whole genome shotgun sequence;
754788309; NZ_BBN001000002.1
2040; Streptomyces sp. NBRC 110027, whole genome shotgun sequence;
754796661; NZ_BBN001000008.1
2041; Paenibacillus sp. FSL R7-0331, complete genome; 754821094;
NZ_CP009284.1
2042; Kibdelosporangium sp. MJ126-NF4, whole genome shotgun sequence;
754819815; NZ_CDME01000002.1
2043; Paenibacillus camerounensis strain G4, whole genome shotgun sequence;
754841195; NZ_CCDG010000069.1
2044; Paenibacillus borealis strain DSM 13188, complete genome; 754859657;
NZ_CP009285.1
2045; Legionella pneumophila serogroup 1 strain TUM 13948, whole genome
shotgun sequence; 754875479; NZ_BAYQ01000013.1
2046; Streptacidiphilus neutrinimicus strain NBRC 100921, whole genome
shotgun sequence; 755016073; NZ_BBP001000030.1
2047, Streptacidiphilus melanogenes strain NBRC 103184, whole genome
shotgun sequence; 755032408; NZ_BBPP01000024.1
2048, Streptacidiphilus anmyonensis strain NBRC 103185, whole genome
shotgun sequence; 755077919; NZ_BBPQ01000048.1
2049, Streptacidiphilus jiangxiensis strain NBRC 100920, whole genome shotgun
sequence; 755108320; NZ_BBPN01000056.1
2050; Mesorhizobium sp. ORS3359, whole genome shotgun sequence;
756828038; NZ_CCNC01000143.1
2051; Bacillus megaterium WSH-002, complete genome; 384044176;
NC_017138.1
2052; Aneurinibacillus migulanus strain Nagano E1 contig_36, whole genome
shotgun sequence; 928874573; NZ_LIXL01000208.1
2053; Sphingobium sp. Ant17 Contig_90, whole genome shotgun sequence;
759431957; NZ_JEMV01000094.1
2054; Pseudomonas sp. HMP271 Pseudomonas HMP271_contig_7,
genome shotgun sequence; 759578528; NZ_JMFZ01000007.1
2055; Streptomyces luteus strain TRM 45540 Scaffoldl, whole genome shotgun
sequence; 759659849; NZ_KNO39946.1
2056; Streptomyces nodosus strain ATCC 14899 genome; 759739811;
NZ_CP009313.1
2057; Streptomyces fradiae strain ATCC 19609 contig0008, whole genome
shotgun sequence; 759752221; NZ_JNAD01000008.1
2058; Streptomyces bingchenggensis BCW-1, complete genome; 374982757;
NC_016582.1
2059; Streptomyces glaucescens strain GLA.O, complete genome; 759802587;
NZ_CP009438.1
2060; Novosphingobium sp. Rr 2-17 contig98, whole genome shotgun sequence;
393773868; NZ_AKFJ01000097.1
2061; Nonomumea candida strain NRRL B-24552 contig27.1, whole genome
shotgun sequence; 759944049; NZ_JOAG01000029.1
2062; Nonomumea candida strain NRRL B-24552 contig28.1, whole genome
shotgun sequence; 759944490; NZ_JOAG01000030.1
2063; Nonomumea candida strain NRRL B-24552 contig42.1, whole genome
shotgun sequence; 759948103; NZ_JOAG01000045.1
2064; Paenibacillus polymyxa E681, complete genome; 864439741; NC_014483.2
2065; Xanthomonas hortorum pv. carotae str. M081 chromosome, whole genome
shotgun sequence; 565808720; NZ_CM002307.1
2066; Novosphingobium sp. P6W scaffold3, whole genome shotgun sequence;
763092879; NZ_JXZE01000003.1
2067; Novosphingobium sp. P6W scaffold9, whole genome shotgun sequence;
763095630; NZ_JXZE01000009.1
2068; Sphingomonas hengshuiensis strain WHSC-8, complete genome;
764364074; NZ_CP010836.1
2069; Streptomyces ahygroscopicus subsp. wuyiensis strain CK-15 contig3, whole
lgenome shotgun sequence; 921220646;
2070; Streptomyces cyaneogriseus subsp. noncyanogenus strain NMWT 1,
complete genome; 764487836; NZ_CP010849.1
2071; Bacillus subtilis subsp. spizizenii RFWG1A4 contig00010, whole genome
shotgun sequence; 764657375; NZ_AJHM01000010.1
2072; Mastigocladus laminosus UU774 scaffold 22, whole genome shotgun
sequence; 764671177; NZ_JX1101000139.1
2073; Mooreaproducens 3L scf52052, whole genome shotgun sequence;
332710285; NZ_GL890953.1
2074; Streptomyces iranensis genome assembly Siranensis, scaffold SCAF00002;
765016627; NZ_LK022849.1
2075; Risungbinella massiliensis strain GD1, whole genome shotgun sequence;
765315585; NZ_LN812103.1
2076; Sphingobium sp. YBL2, complete genome; 765344939; NZ_CP010954.1
2077; Streptococcus suis strain LS5J, whole genome shotgun sequence;
765394696; NZ_CEEZ01000028.1
2078; Streptococcus suis strain LS8I, whole genome shotgun sequence;
766595491; NZ_CEHM01000004.1
2079; Thalassospira sp. HJ NODE 2, whole genome shotgun sequence;
766668420; NZ_JY1101000010.1
2080; Frankia sp. CpIl-P FF86_1013, whole genome shotgun sequence;
946950294; NZ_LEX01000013.1
2081; Streptococcus suis strain B28P, whole genome shotgun sequence;
769231516; NZ_CDTB01000010.1
2082; Streptomyces sp. NRRL F-4428 contig40.2, whole genome shotgun
sequence; 772774737; NZ_JYJI01000131.1
2083; Bacterium endosymbiont of Mortierella elongata FMR23-6, whole genome
shotgun sequence; 779889750; NZ_DF850521.1
2084; Streptomyces sp. FxanaA7 F611DRAFT_scaffold00041.41_C, whole
genome shotgun sequence; 780340655; NZ_LACL01000054.1
2085; Streptomyces rubellomurinus strain ATCC 31215 contig-63, whole genome
shotgun sequence; 783211546; NZ_JZKH01000064.1
2086; Streptomyces rubellomurinus subsp. indigoferus strain ATCC 31304 contig-55,
whole genome shotgun sequence; 783374270; NZ_JZKG01000056.1
2087; Bacillus sp. UMTAT18 contig000011, whole genome shotgun sequence;
NZ_PM02000059.1 806951735; NZ_JSFD01000011.1
2088; Paenibacillus wulumuqiensis strain Y24 Scaffold4, whole genome shotgun
sequence; 808051893; NZ_KQ040793.1
2089; Paenibacillus daici strain H9 Scaffold3, whole genome shotgun sequence;
808064534; NZ_KQ040798.1
2090; Paenibacillus algorifonticola strain XJ259 Scaffold20_1, whole genome
shotgun sequence; 808072221; NZ_LAQ001000025.1
2091; Xanthomonas campestris strain 17, complete genome; 810489403;
NZ_CP011256.1
2092; Bacillus sp. SA1-12 scf7180000003378, whole genome shotgun sequence;
817541164; NZ_LATZ01000026.1
2093; Spirosoma radiotolerans strain DG5A, complete genome; 817524426;
NZ_CP010429.1
2094; Streptomyces lydicus A02, complete genome; 822214995;
NZ_CP007699.1
2095; Streptomyces lydicus A02, complete genome; 822214995;
NZ_CP007699.1
2096; Bacillus cereus strain B4147 NODE_5, whole genome shotgun sequence;
822530609; NZ_LCYN01000004.1
2097; Xanthomonas pisi DSM 18956 Contig_28, whole genome shotgun
sequence; 822535978; NZ_JPLE01000028.1
2098; Erythrobacter luteus strain KA37 contig1, whole genome shotgun sequence;
822631216; NZ_LBHB01000001.1
2099; Xanthomonas arboricola strain CFBP 7634 Xarjug-CFBP7634-G11, whole
genome shotgun sequence; 825139250; NZ_JZEH01000001.1
2100; Xanthomonas arboricola strain CFBP 7651 Xarjug-CFBP7651-G11, whole
genome shotgun sequence; 825156557; NZ_JZEI01000001.1
2101; Luteimonas sp. FCS-9 scf7180000000225, whole genome shotgun
sequence; 825314716; NZ_LASZ01000002.1
2102; Streptomyces sp. KE1 Contig11, whole genome shotgun sequence;
825353621; NZ_LAYX01000011.1
2103; Streptomyces sp. M10 Scaffold2, whole genome shotgun sequence;
835355240; NZ_KN549147.1
2104; Xanthomonas cannabis pv. phaseoli strain Nyagatare scf 52938_7, whole
genome shotgun sequence; 835885587; NZ_KN265462.1
2105; Bacillus aryabhattai strain T61 Scaffold1, whole genome shotgun sequence;
836596561; NZ_KQ087173.1
2106; Paenibacillus sp. TCA20, whole genome shotgun sequence; 843088522;
NZ_BBIWO1000001.1
2107; Bacillus circulans strain RIT379 contig11, whole genome shotgun sequence;
844809159; NZ_LDPH01000011.1
2108; Omithinibacillus califomiensis strain DSM 16628 contig_22, whole genome
shotgun sequence; 849059098; NZ_LDUE01000022.1
2109; Bacillus pseudalcaliphilus strain DSM 8725 super11, whole genome
shotgun sequence; 849078078;
2110; Bacillus aryabhattai strain LK25 16, whole genome shotgun sequence;
850356871; NZ_LDWN01000016.1
2111; Methanobactenum arcticum strain M2 EI99DRAFT_scaffold00005.5_C,
whole genome shotgun sequence; 851140085; NZ_JQKN01000008.1
2112; Methanobacterium sp. SMA-27 DL91DRAFT_unitig_0_quiver.1_C, whole
genome shotgun sequence; 851351157; NZ_JQLY01000001.1
2113; Cellulomonas sp. A375-1 contig_129, whole genome shotgun sequence;
856992287; NZ_LFKW01000127.1
2114; Streptomyces sp. HNS054 contig28, whole genome shotgun sequence;
860547590; NZ_LDZX01000028.1
2115; Bacillus cereus strain RIMV BC 126 212, whole genome shotgun sequence;
872696015; NZ_LAB001000035.1
2116; Sphingomonas sp. MEA3-1 contig00021, whole genome shotgun sequence;
873296042; NZ_LECE01000021.1
2117; Sphingomonas sp. MEA3-1 contig00040, whole genome shotgun sequence;
873296160; NZ_LECE01000040.1
2118; Bacillus sp. 220_BSPC 1447_75439_1072255, whole genome shotgun
sequence; 880954155; NZ_JVPL01000109.1
2119; Bacillus sp. 522_BSPC 2470_72498_1083579_594_ . . . _522_, whole
genome shotgun sequence; 880997761; NZ_JVDT01000118.1
2120; Streptomyces ipomoeae 91-03 gcontig_1108499710267, whole genome
shotgun sequence; 429195484; NZ_AEJC01000118.1
2121; Scytonema tolypothlichoides VB-61278 scaffold 6, whole genome shotgun
sequence; 890002594; NZ_JXCA01000005.1
2122; Erythrobacter atlanticus strain s21-N3, complete genome; 890444402;
NZ_CP011310.1
2123; Sphingobium yanoikuyae strain SHJ scaffold12, whole genome shotgun
sequence; 893711343; NZ_KQ235994.1
2124; Sphingobium yanoikuyae strain SHJ scaffold33, whole genome shotgun
sequence; 893711364; NZ_KQ236015.1
2125; Sphingobium yanoikuyae strain SHJ scaffold47, whole genome shotgun
sequence; 893711378; NZ_KQ236029.1
2126; Stenotrophomonas maltophilia strain 544_SMAL
1161_223966_2976806_599_ . . . _882_, whole genome shotgun sequence;
896492362; NZ_JVCU01000107.1 NZ_LFJ001000006.1
2127; Stenotrophomonas maltophilia strain 131_SMAL
1126_236170_8501292_717_ . . . _1018_, whole genome shotgun sequence;
896520167; NZ_JVUI01000038.1
2128; Stenotrophomonas maltophilia strain 95l_SMAL 71_125859_2268311,
whole genome shotgun sequence; 896567682; NZ_JUMH01000022.1
2129; Stenotrophomonas maltophilia strain OC194 contig_98, whole genome
shotgun sequence; 930169273; NZ_LEH01000098.1
2130; Streptococcus pseudopneumoniae strain 445_SPSE
347_91401_2272315_31_ . . . _319_, whole genome shotgun sequence;
896667361; NZ_JVGV01000030.1
2131; Streptomyces caatingaensis strain CMAA 1322 contig02, whole genome
shotgun sequence; 906344334; NZ_LFXA01000002.1
2132; Streptomyces caatingaensis strain CMAA 1322 contig02, whole genome
shotgun sequence; 906344334; NZ_LFXA01000002.1
2133; Streptomyces caatingaensis strain CMAA 1322 contig07, whole genome
shotgun sequence; 906344339; NZ_LFXA01000007.1
2134; Sphingopyxis alaskensis RB2256, complete genome; 103485498;
NC_008048.1
2135; Sphingomonas wittichii RW1, complete genome; 148552929;
NC_009511.1
2136; Caulobacter sp. K31, complete genome; 167643973; NC_010338.1
2137; Asticcacaulis excentricus CB 48 chromosome 2, complete sequence;
315499382; NC_014817.1
2138; Nocardiopsis dassonvillei subsp. dassonvillei DSM 43111 chromosome 1,
complete sequence; 297558985; NC_014210.1
2139; Streptomyces wadayamensis strain A23 LGO_A2_AS7_C00257, whole
genome shotgun sequence; 910050821; NZ_JHDU01000034.1
2140; Tolypothrix bouteillei VB521301 scaffold_1, whole genome shotgun
sequence; 910242069; NZ_JHEG02000048.1
2141; Silvibacterium bohemicum strain S15 contig_3, whole genome shotgun
sequence; 910257956; NZ_LBHJ01000003.1
2142; Silvibacterium bohemicum strain S15 contig_3, whole genome shotgun
sequence; 910257956; NZ_LBHJ01000003.1
2143; Silvibacterium bohemicum strain S15 contig_30, whole genome shotgun
sequence; 910257973; NZ_LBHJ01000020.1
2144; Streptomyces sp. NRRL WC-3773 contig11.1, whole genome shotgun
sequence; 664481891; NZ_JOJI01000011.1
2145; Streptomyces peucetius strain NRRL WC-3868 contig49.1, whole genome
shotgun sequence; 665671804; NZ_JOCK01000052.1
2146; Xanthomonas citri pv. mangiferaeindicae LMG 941, whole genome shotgun
sequence; 381171950; NZ_CAH001000029.1
2147; Mesorhizobium sp. L2C084A000 scaffold0007, whole genome shotgun
sequence; 563938926; NZ_AYWX01000007.1
2148; Erythrobacter citreus LAMA 915 Contig13, whole genome shotgun
sequence; 914607448; NZ_JYNE01000028.1
2149; Bacillus flexus strain Riq5 contig_32, whole genome shotgun sequence;
914730676;NZ_LFQJ01000032.1
2150; Rhodanobacter thiooxydans LCS2 contig057, whole genome shotgun
sequence; 389809081; NZ_AJXWO1000057.1
2151; Frankia alni str. ACN14A chromosome, complete sequence; 111219505;
NC_008278.1
2152; Novosphingobium sp. PP1Y main chromosome, complete replicon;
334139601; NC_015580.1
2153; Salinibacter ruber M8 chromosome, complete genome; 294505815;
NC_014032.1
2154; Nocardiopsis sauna YIM 90010 contig_87, whole genome shotgun
sequence; 484023389; NZ_ANBF01000087.1
2155; Kitasatospora setae KM-6054 DNA, complete genome; 357386972;
NC_016109.1
2156; Arthrobacter sp. 161MFSha2.1 C567DRAFT_scaffold00006.6, whole
genome shotgun sequence; 484021228; NZ_KB895788.1
2157; Lamprocystis purpura DSM 4197 A390DRAFT_scaffold_01, whole
genome shotgun sequence; 483254584; NZ_KB902362.1
2158; Streptomyces sp. ATexAB-D23 B082DRAFT_scaffold_01, whole genome
shotgun sequence; 483975550; NZ_KB892001.1
2159; Lunatimonas lonarensis strain AK24 S14_contig_18, whole genome
shotgun sequence; 499123840; NZ_AQHR01000021.1
2160; Amycolatopsis benzoatilytica AK 16/65 AmybeDRAFT_scaffold1.1, whole
genome shotgun sequence; 486399859; NZ_KB912942.1
2161; Nocardia transvalensis NBRC 15921, whole genome shotgun sequence;
485125031; NZ_BAGL01000055.1
2162; Sphingomonas sp. YL-JM2C contig056, whole genome shotgun sequence;
661300723; NZ_ASTM01000056.1
2163; Butyrivibrio sp. XBB1001 G631DRAFT_scaffold00005.5_C, whole
genome shotgun sequence; 651376721; NZ_AUKA01000006.1
2164; Butyrivibrio fibrisolvens MD2001 G635DRAFT scaffold00033.33_C,
whole genome shotgun sequence; 652963937; NZ_AUKD01000034.1
2165; Butyrivibrio sp. NC3005 G634DRAFT scaffold00001.1, whole genome
shotgun sequence; 651394394; NZ_KE384206.1
2166; Shimazuella kribbensis DSM 45090 A3GQDRAFT_scaffold_0.1S, whole
genome shotgun sequence; 655370026; NZ_ATZFO1000001.1
2167; Shimazuella kribbensis DSM 45090 A3GQDRAFT_scaffold_5.6_C, whole
genome shotgun sequence; 655371438; NZ_ATZFO1000006.1
2168; Desulfobulbus mediterraneus DSM 13871 G494DRAFT_scaffold00028.28_C,
whole genome shotgun sequence; 655138083; NZ_AUCW01000035.1
2169; Cohnella thennotolerans DSM 17683 G485DRAFT_scaffold00041.41_C,
whole genome shotgun sequence; 652787974; NZ_AUCP01000055.1
2170; Azospirillum halopraeferens DSM 3675 G472DRAFT_scaffold00039.39_C,
whole genome shotgun sequence; 655967838; NZ_AUCF01000044.1
2171; Bacillus kribbensis DSM 17871 H539DRAFT_scaffold00003.3, whole
genome shotgun sequence; 651983111; NZ_KE387239.1
2172; Leptolyngbya sp. Heron Island J 67, whole genome shotgun sequence;
553740975; NZ_AWNH01000084.1
2173; Streptomyces sp. GXT6 genomic scaffold Scaffold4, whole genome
shotgun sequence; 654975403; NZ_KI601366.1
2174; Promicromonospora kroppenstedtii DSM 19349 ProkrDRAFT_PKA.71,
whole genome shotgun sequence; 739097522; NZ_KI911740.1
2175; Bacillus sp. J37 BacJ37DRAFT_scaffold_0.1S, whole genome shotgun
sequence; 651516582; NZ_JAEK01000001.1
2176; Prevotella oryzae DSM 17970 XylorDRAFT_X0A.1, whole genome
shotgun sequence; 738999090; NZ_KK073873.1
2177; Sphingobium sp. Ant17 Contig_45, whole genome shotgun sequence;
759429528; NZ_JEMV01000036.1
2178; Rubellimicrobium mesophilum DSM 19309 scaffold23, whole genome
shotgun sequence; 739419616; NZ_KK088564.1
2179; Butyrivibrio sp. MC2021 T359DRAFT_scaffold00010.10_C, whole
genome shotgun sequence; 651407979; NZ_JHXX01000011.1
2180; Clostridium beijerinckii HUN142 T483DRAFT_scaffold00004.4, whole
genome shotgun sequence; 652494892; NZ_KK211337.1
2181; Streptomyces sp. Tu 6176 scaffold00003, whole genome shotgun sequence;
740044478; NZ_KK106990.1
2182; Novosphingobium resinovorum strain KF1 contig000008, whole genome
shotgun sequence; 738615271; NZ_JFYZ01000008.1
2183; Novosphingobium resinovorum strain KF1 contig000015, whole genome
shotgun sequence; 738617000; NZ_JFYZ01000015.1
2184; Hyphomonas chukchiensis strain BH-BN04-4 contig29, whole genome
shotgun sequence; 736736050; NZ_AWFG01000029.1
2185; Thioclava dalianensis strain DLFJ1-1 contig2, whole genome shotgun
sequence; 740220529; NZ_JHEH01000002.1
2186; Thioclava indica strain DT23-4 contig29, whole genome shotgun sequence;
740292158; NZ_AUNB01000028.1
2187; Streptomyces albus subsp. albus strain NRRL B-1811 contig32.1, whole
genome shotgun sequence; 665618015; NZ_JODR01000032.1
2188; Kitasatospora sp. MBT66 scaffold3, whole genome shotgun sequence;
759755931; NZ_JAIY01000003.1
2189; Sphingomonas sp. DC-6 scaffold87, whole genome shotgun sequence;
662140302; NZ_JMUB01000087.1
2190; Sphingobium chlorophenolicum strain NBRC 16172 contig000062, whole
genome shotgun sequence; 739598481; NZ_JFHR01000062.1
2191; Nocardia sp. NRRL WC-3656 contig2.1, whole genome shotgun sequence;
663737675; NZ_JOJF01000002.1
2192; Streptomyces flavochromogenes strain NRRL B-2684 contig8.1, whole
genome shotgun sequence; 663317502; NZ_JNZ001000008.1
2193; Bacillus indicus strain DSM 16189 Contig01, whole genome shotgun
sequence; 737222016; NZ_JNVC02000001.1
2194; Streptomyces bicolor strain NRRL B-3897 contig42.1, whole genome
shotgun sequence; 671498318; NZ_JOFR01000042.1
2195; Streptomyces sp. NRRL WC-3719 contig152.1, whole genome shotgun
sequence; 665536304; NZ_JOCD01000152.1
2196; Streptomyces sp. NRRL F-5053 contig1.1, whole genome shotgun
sequence; 664356765; NZ_JOHT01000001.1
2197; Streptomyces sp. NRRL S-1868 contig54.1, whole genome shotgun
sequence; 664360925; NZ_JOGD01000054.1
2198; Streptomyces hygroscopicus subsp. hygroscopicus strain NRRL B-1477
contig8.1, whole genome shotgun sequence; 664299296; NZ_JOIK01000008.1
2199; Desulfobacter vibrioformis DSM 8776 Q366DRAFT_scaffold00036.35_C,
whole genome shotgun sequence; 737257311; NZ_JQKJ01000036.1
2200; Brevundimonas sp. EAKA contig5, whole genome shotgun sequence;
737322991; NZ_JMQR01000005.1
2201; Brevundimonas sp. EAKA contig5, whole genome shotgun sequence;
737322991; NZ_JMQR01000005.1
2202; Actinokineospora spheciospongiae strain EG49 contig1268_1, whole
genome shotgun sequence; 737301464; NZ_AYXG01000139.1
2203; Sphingobium sp. bal seq0028, whole genome shotgun sequence;
739622900; NZ_JPPQ01000069.1
2204; Rothia dentocariosa strain C6B contig_5, whole genome shotgun sequence;
739372122; NZ_JQHE01000003.1
2205; Rhodococcus fascians A21d2 contig10, whole genome shotgun sequence;
739287390; NZ_JMFA01000010.1
2206; Rhodococcus fascians LMG 3625 contig38, whole genome shotgun
sequence; 694033726; NZ_JMEM01000016.1
2207; Sphingopyxis sp. MWB1 contig00002, whole genome shotgun sequence;
696542396; NZ_JQFJ01000002.1
2208; Sphingobium yanoikuyae strain B1 scaffold1, whole genome shotgun
sequence; 739650776; NZ_KL662193.1
2209; Lysobacter daejeonensis GH1-9 contig23, whole genome shotgun sequence;
738180952; NZ_AVPU01000014.1
2210; Sphingomonas sp. 35-24ZXX contig11_scaffold4, whole genome shotgun
sequence; 728827031; NZ_JR0G01000008.1
2211; Sphingomonas sp. 37zxx contig3_scaffold2, whole genome shotgun
sequence; 728813405; NZ_JR0H01000003.1
2212; Actinoalloteichus spitiensis RMV-1378 Contig406, whole genome shotgun
sequence; 483112234; NZ_AGVX02000406.1
2213; Alistipes sp. ZOR0009 L990_140, whole genome shotgun sequence;
835319962; NZ_JTLD01000119.1
2214; Sphingopyxis sp. LC363 contig36, whole genome shotgun sequence;
739702045; NZ_JNFC01000030.1
2215; Sphingopyxis sp. LC81 contig24, whole genome shotgun sequence;
739659070; NZ_JNFD01000017.1
2216; Sphingomonas sp. Ant H11 contig_149, whole genome shotgun sequence;
730274767; NZ_JSBN01000149.1
2217; Novosphingobium malaysiense strain MUSC 273 Contig_11, whole genome
shotgun sequence; 746242072; NZ_JIDI01000011.1
2218; Novosphingobium subtenaneum strain DSM 12447 NJ75_contig000028,
whole genome shotgun sequence; 746290581; NZ_JRVC01000028.1
2219; Brevundimonas nasdae strain TPW30 Contig_13, whole genome shotgun
sequence; 746187665; NZ_JWSY01000013.1
2220; Desulfosporosinus youngiae DSM 17734 chromosome, whole genome
shotgun sequence; 374578721; NZ_CM001441.1
2221; Rivularia sp. PCC 7116, complete genome; 427733619; NC_019678.1
2222; Gorillibacterium massiliense strain G5, whole genome shotgun sequence;
750677319; NZ_CBQR020000171.1
2223; Nonomumea candida strain NRRL B-24552 contig8 1, whole genome
shotgun sequence; 759934284; NZ_JOAG01000009.1
2224; Mesorhizobium sp. SOD10, whole genome shotgun sequence; 751285871;
NZ_CCNA01000001.1
2225; Citrobacter pasteurii strain CIP 55.13, whole genome shotgun sequence;
749611130; NZ_CDHL01000044.1
2226; Cohnella kolymensis strain VKM B-2846 B2846_22, whole genome
shotgun sequence; 751596254; NZ_JXAL01000022.1
2227; Jeotgalibacillus campisalis strain SF-57 contig00001, whole genome
shotgun sequence; 751586078; NZ_ARR01000001.1
2228; Closltidium beijerinckii strain NCIMB 14988 genome; 754484184;
NZ_CP010086.1
2229; Novosphingobium sp. P6W scaffold17, whole genome shotgun sequence;
763097360; NZ_JXZE01000017.1
2230; Sphingomonas hengshuiensis strain WHSC-8, complete genome;
764364074; NZ_CP010836.1
2231; Sphingobium sp. YBL2, complete genome; 765344939; NZ_CP010954.1
2232; Methanobacterium formicicum genome assembly DSM1535,
chromosome: chr1; 851114167; NZ_LN515531.1
2233; Bacillus cereus genome assembly Bacillus JRS4, contig contig000025,
whole genome shotgun sequence; 924092470; CYHM01000025.1
2234; Frankia sp. DC12 FraDC12DRAFT_scaffold1.1, whole genome shotgun
sequence; 797224947; NZ_KQ031391.1
2235; Closltidium scatologenes strain ATCC 25775, complete genome;
802929558; NZ_CP009933.1
2236; Sphingomonas sp. SRS2 contig40, whole genome shotgun sequence;
806905234; NZ_LARW01000040.1
2237; Jiangella alkaliphila strain KCTC 19222 Scaffold1, whole genome shotgun
sequence; 820820518; NZ_KQ061219.1
2238; Erythrobacter marinus strain HWDM-33 contig3, whole genome shotgun
sequence; 823659049; NZ_LBHU01000003.1
2239; Luteimonas sp. FCS-9 scf7180000000226, whole genome shotgun
sequence; 825314728; NZ_LASZ01000003.1
2240; Sphingomonas parapaucimobilis NBRC 15100 BBPI01000030, whole
genome shotgun sequence; 755134941; NZ_BBPI01000030.1
2241; Sphingobium barthaii strain KK22, whole genome shotgun sequence;
646523831; NZ_BATN01000047.1
2242; Erythrobacter matinus strain HWDM-33 contig3, whole genome shotgun
sequence; 823659049; NZ_LBHU01000003.1
2243; Streptomyces avicenniae strain NRRL B-24776 contig3.1, whole genome
shotgun sequence; 919531973; NZ_JOEK01000003.1
2244; Sphingomonas sp. Y57 scaffold74, whole genome shotgun sequence;
826051019; NZ_LDES01000074.1
2245; Xanthomonas campestris strain CFSAN033089 contig_46, whole genome
shotgun sequence; 920684790; NZ_LHBW01000046.1
2246; Croceicoccus naphthovorans strain PQ-2, complete genome; 836676868;
NZ_CP011770.1
2247; Streptomyces caatingaensis strain CMAA 1322 contig09, whole genome
shotgun sequence; 906344341; NZ_LFXA01000009.1
2248; Paenibacillus sp. FJAT-27812 scaffold_0, whole genome shotgun sequence;
922780240; NZ_LIGH01000001.1
2249; Stenotrophomonas maltophilia strain ISMMS2R, complete genome;
923060045; NZ_CP011306.1
2250; Stenotrophomonas maltophilia strain ISMMS3, complete genome;
923067758; NZ_CP011010.1
2251; Hapalosiphon sp. MRB220 contig_91, whole genome shotgun sequence;
923076229; NZ_LIRN01000111.1
2252; Stenotrophomonas maltophilia strain B4 contig779, whole genome shotgun
sequence; 924516300; NZ_LDVR01000003.1
2253; Bacillus sp. FJAT-21352 Scaffold1, whole genome shotgun sequence;
924654439; NZ_LIU501000003.1
2254; Sphingopyxis sp. 113P3, complete genome; 924898949; NZ_CP009452.1
2255; Sphingopyxis sp. 113P3, complete genome; 924898949; NZ_CP009452.1
2256; Streptomyces sp. CFMR 7 strain CFMR-7, complete genome; 924911621;
NZ_CP011522.1
2257; Bacillus gobiensis strain FJAT-4402 chromosome; 926268043;
NZ_CP012600.1
2258; Streptomyces sp. XY431 P412contig111.1, whole genome shotgun
sequence; 926317398; NZ_LGDO01000015.1
2259; Streptomyces sp. NRRL F-6491 P443contig15.1, whole genome shotgun
sequence; 925610911; LGEE01000058.1
2260; Streptomyces sp. NRRL B-1140 P439contig15.1, whole genome shotgun
sequence; 926344107; NZ_LGEA01000058.1
2261; Streptomyces sp. NRRL B-1140 P439contig32.1, whole genome shotgun
sequence; 926344331; NZ_LGEA01000105.1
2262; Streptomyces sp. NRRL F-5755 P309contig48.1, whole genome shotgun
sequence; 926371517; NZ_LGCW01000271.1
2263; Streptomyces sp. NRRL F-5755 P309contig7.1, whole genome shotgun
sequence; 926371541; NZ_LGCW01000295.1
2264; Streptomyces sp. WM6378 P402contig63.1, whole genome shotgun
sequence; 926403453; NZ_LGDD01000321.1
2265; Streptomyces sp. WM6378 P402contig63.1, whole genome shotgun
sequence; 926403453; NZ_LGDD01000321.1
2266; Nocardia sp. NRRL S-836 P437contig39.1, whole genome shotgun
sequence; 926412104; NZ_LGDY01000113.1
2267; Paenibacillus sp. A59 contig_353, whole genome shotgun sequence;
927084730; NZ_LITU01000050.1
2268; Paenibacillus sp. A59 contig_416, whole genome shotgun sequence;
927084736; NZ_L1TU01000056.1
2269; Streptomyces sp. NRRL S-444 contig322.4, whole genome shotgun
sequence; 797049078; JZWX01001028.1
2270; Altererythrobacter atlanticus strain 26DY36, complete genome; 927872504;
NZ_CP011452.2
2271; Streptomyces chattanoogensis strain NRRL ISP-5002 ISP5002contig8.1,
whole genome shotgun sequence; 928897585; NZ_LGKG01000196.1
2272; Streptomyces chattanoogensis strain NRRL ISP-5002 ISP5002contig9.1,
whole genome shotgun sequence; 928897596; NZ_LGKG01000207.1
2273; Ideonella sakaiensis strain 201-F6, whole genome shotgun sequence;
928998724; NZ_BBYR01000007.1
2274; Ideonella sakaiensis strain 201-F6, whole genome shotgun sequence;
928998800; NZ_BBYR01000083.1
2275; Bacillus sp. FJAT-28004 scaffold 2, whole genome shotgun sequence;
929005248; NZ_LGHP01000003.1
2276; Novosphingobium sp. AAP1 AAP1Contigs7, whole genome shotgun
sequence; 930029075; NZ_LJHO01000007.1
2277; Novosphingobium sp. AAP1 AAP1Contigs9, whole genome shotgun
sequence; 930029077; NZ_LJHO01000009.1
2278; Actinobacteria bacterium OK074 ctg60, whole genome shotgun sequence;
930473294; NZ_LJCV01000275.1
2279; Actinobacteria bacterium OK006 ctg112, whole genome shotgun sequence;
930490730; NZ_UCUO1000014.1
2280; Frankia sp. R43 contig001, whole genome shotgun sequence; 937182893;
NZ_LFCW01000001.1
2281; Sphingopyxis macrogoltabida strain EY-1, complete genome; 937372567;
NZ_CP012700.1
2282; Xanthomonas arboricola strain CITA 44 CITA_44_contig_26, whole
genome shotgun sequence; 937505789; NZ_LJGM01000026.1
2283; Stenotrophomonas acidaminiphila strain ZAC14D2 NAIMI4 2, complete
genome; 938883590; NZ_CP012900.1
2284; Sphingopyxis macrogoltabida strain 203, complete genome; 938956730;
NZ_CP009429.1
2285; Sphingopyxis macrogoltabida strain 203, complete genome; 938956730;
NZ_CP009429.1
2286; Sphingopyxis macrogoltabida strain 203 plasmid, complete sequence;
938956814; NZ_CP009430.1
2287; Cellulosilyticum ruminicola JCM 14822, whole genome shotgun sequence;
938965628; NZ_BBCG01000065.1
2288; Brevundimonas sp. DS20, complete genome; 938989745; NZ_CP012897.1
2289; Brevundimonas sp. DS20, complete genome; 938989745; NZ_CP012897.1
2290; Paenibacillus sp. GD6, whole genome shotgun sequence; 939708098;
NZ_LN831198.1
2291; Paenibacillus sp. GD6, whole genome shotgun sequence; 939708105;
NZ_LN831205.1
2292; Alicyclobacillus ferrooxydans strain TC-34 contig_22, whole genome
shotgun sequence; 940346731; NZ_LJC001000107.1
2293; Xanthomonas sp. Mitacek01 contig_17, whole genome shotgun sequence;
941965142; NZ_LKIT01000002.1
2294; Streptomyces bingchenggensis BCW-1, complete genome; 374982757;
NC_016582.1
2295; Streptomyces pactum strain ACT12 scaffold1, whole genome shotgun
sequence; 943388237; NZ_LIQD01000001.1
2296; Streptomyces flocculus strain NRRL B-2465 B2465_contig_205, whole
genome shotgun sequence; 943674269; NZ_LIQO01000205.1
2297; Streptomyces aurantiacus strain NRRL ISP-5412 ISP-5412_contig_138,
whole genome shotgun sequence; 943881150; NZ_LIPP01000138.1
2298; Streptomyces graminilatus strain NRRL B-59124 B59124_contig_7, whole
genome shotgun sequence; 943897669; NZ_LIQQ01000007.1
2299; Streptomyces alboniger strain NRRL B-1832 B-1832_contig_37, whole
genome shotgun sequence; 943898694; NZ_LIQN01000037.1
2300; Streptomyces alboniger strain NRRL B-1832 B-1832_contig_384, whole
genome shotgun sequence; 943899498; NZ_LIQN01000384.1
2301; Streptomyces kanamyceticus strain NRRL B-2535 B-2535_contig_122,
whole genome shotgun sequence; 943922224; NZ_LIQUo1000122.1
2302; Streptomyces luridiscabiei strain NRRL B-24455 B24455 contig_315,
whole genome shotgun sequence; 943927948; NZ_LIQV01000315.1
2303; Streptomyces attiruber strain NRRL B-24165 contig_124, whole genome
shotgun sequence; 943949281; NZ_LIPN01000124.1
2304; Streptomyces hirsutus strain NRRL B-2713 B2713_contig_57, whole
genome shotgun sequence; 944005810; NZ_LIQT01000057.1
2305; Streptomyces aureus strain NRRL B-2808 contig_171, whole genome
shotgun sequence; 944012845; NZ_LIPQ01000171.1
2306; Streptomyces phaeochromogenes strain NRRL B-1248 B-1248_contig_126,
whole genome shotgun sequence; 944029528; NZ_LIQZ01000126.1
2307; Streptomyces torulosus strain NRRL B-3889 B-3889_contig_18, whole
genome shotgun sequence; 944495433; NZ_LIRK01000018.1
2308; Frankia alni str. ACN14A chromosome, complete sequence; 111219505;
NC_008278.1
2309; Sphingomonas sp. Leaf20 contig_1, whole genome shotgun sequence;
947349881; NZ_LMKN01000001.1
2310; Paenibacillus sp. Leaf72 contig_6, whole genome shotgun sequence;
947378267; NZ_LMLV01000032.1
2311; Sphingomonas sp. Leaf230 contig_4, whole genome shotgun sequence;
947401208; NZ_LMKW01000010.1
2312; Sanguibacter sp. Leaf3 contig_2, whole genome shotgun sequence;
947472882; NZ_LMRH01000002.1
2313; Aeromicrobium sp. Root344 contig_1, whole genome shotgun sequence;
947552260; NZ_LMDH01000001.1
2314; Sphingopyxis sp. Root1497 contig_3, whole genome shotgun sequence;
947689975; NZ_LMGF01000003.1
2315; Sphingomonas sp. Root720 contig_7, whole genome shotgun sequence;
947704642; NZ_LMID01000015.1
2316; Sphingomonas sp. Root720 contig_8, whole genome shotgun sequence;
947704650; NZ_LMID01000016.1
2317; Sphingomonas sp. Root710 contig_1, whole genome shotgun sequence;
947721816; NZ_LM1B01000001.1
2318; Mesorhizobium sp. Root172 contig_2, whole genome shotgun sequence;
947919015; NZ_LMHP01000012.1
2319; Mesorhizobium sp. Root102 contig_3, whole genome shotgun sequence;
947937119; NZ_LMCP01000023.1
2320; Paenibacillus sp. Soil750 contig_1, whole genome shotgun sequence;
947966412; NZ_LMSD01000001.1
2321; Paenibacillus sp. Soi1522 contig_3, whole genome shotgun sequence;
947983982; NZ_LMRV01000044.1
2322; Paenibacillus sp. Root52 contig_3, whole genome shotgun sequence;
948045460; NZ_LMF001000023.1
2323; Bacillus sp. Soi1768D1 contig_5, whole genome shotgun sequence;
950170460; NZ_LMTA01000046.1
2324; Paenibacillus sp. Root444D2 contig_4, whole genome shotgun sequence;
950271971; NZ_LME001000034.1
2325; Paenibacillus sp. Soi1766 contig_32, whole genome shotgun sequence;
950280827; NZ_LMSJ01000026.1
2326; Streptococcus pneumoniae strain type strain: N, whole genome shotgun
sequence; 950938054; NZ_CIHL01000007.1
2327; Streptomyces sp. Root1310 contig_5, whole genome shotgun sequence;
951121600; NZ_LMEQ01000031.1
2328; Bacillus muralis strain DSM 16288 Scaffold4, whole genome shotgun
sequence; 951610263; NZ_LMBV01000004.1
2329; Closliidium butyricum strain KNU-L09 chromosome 1, complete sequence;
959868240; NZ_CP013252.1
2330; Gorillibacterium sp. SN4, whole genome shotgun sequence; 960412751;
NZ_LN881722.1
2331; Thalassobius activus strain CECT 5114, whole genome shotgun sequence;
960424655; NZ_CYUE01000025.1
2332; Microbacterium testaceum strain NS283 contig_37, whole genome shotgun
sequence; 969836538; NZ_LDRU01000037.1
2333; Microbacterium testaceum strain NS183 contig_65, whole genome shotgun
sequence; 969919061; NZ_LDRR01000065.1
2334; Sphingopyxis sp. H050 H050 c0ntig000006, whole genome shotgun
sequence; 970555001; NZ_LNRZ01000006.1
2335; Paenibacillus polymyxa strain KF-1 scaffold00001, whole genome shotgun
sequence; 970574347; NZ_LNZFO1000001.1
2336; Luteimonas abyssi strain XH031 Scaffold1, whole genome shotgun
sequence; 970579907; NZ_KQ759763.1

TABLE 4
Exemplary Lasso Cyclase
Lasso Cyclase Peptide No: #; Species of Origin; GI#; Accession#

2337; Uncultured marine bacterium 463 clone EBAC080-
L32B05 genomic sequence; 41582259; AY458641.2
2338; Burkholderia pseudomallei strain BEF DP42. Contig323,
whole genome shotgun sequence; 686949962; JPNR01000131.1
2339; Burkholderia thailandensis E264 chromosome I,
complete sequence; 83718394; NC_007651.1
2340; Frankia sp. Thr ThrDRAFT_scaffold_48.49, whole
genome shotgun sequence; 602261491; JENI01000049.1
2341; Frankia sp. Thr ThrDRAFT_scaffold_48.49, whole
genome shotgun sequence; 602261491; JENI01000049.1
2342; Sphingopyxis alaskensis RB2256, complete genome;
103485498; NC_008048.1
2343; Sphingopyxis alaskensis RB2256, complete genome;
103485498; NC_008048.1
2344; Streptococcus suis strain LS8I, whole genome shotgun
sequence; 766595491; NZ_CEHM01000004.1
2345; Streptococcus suis SC84 complete genome, strain
SC84; 253750923; NC_012924.1
2346; Geobacter uraniireducens Rf4, complete genome;
148262085; NC_009483.1
2347; Geobacter uraniireducens Rf4, complete genome;
148262085; NC_009483.1
2348; Sphingomonas wittichii RW1, complete genome;
148552929; NC_009511.1
2349; Caulobacter sp. K31, complete genome; 167643973;
NC_010338.1
2350; Phenylobacterium zucineum HLK1, complete
genome; 196476886; CP000747.1
2351; Phenylobacterium zucineum HLK1, complete genome;
196476886; CP000747.1
2352; Sanguibacter keddieii DSM 10542, complete genome;
269793358; NC_013521.1
2353; Xylanimonas cellulosilytica DSM 15894, complete
genome; 269954810; NC_013530.1
2354; Spirosoma linguale DSM 74, complete genome;
283814236; CP001769.1
2355; Stackebrandtia nassauensis DSM 44728, complete
genome; 291297538; NC_013947.1
2356; Caulobacter segnis ATCC 21756, complete genome;
295429362; CP002008.1
2357; Streptomyces bingchenggensis BCW-1, complete genome;
374982757; NC_016582.1
2358; Streptomyces bingchenggensis BCW-1, complete genome;
374982757; NC_016582.1
2359; Gallionella capsifeniformans ES-2, complete genome;
302877245; NC_014394.1
2360; Asticcacaulis excentricus CB 48 chromosome 1, complete
sequence; 315497051; NC_014816.1
2361; Burkholderia gladioli BSR3 chromosome 1, complete
sequence; 327367349; CP002599.1
2362; Mycobacterium sinense strain JDM601, complete
genome; 333988640; NC_015576.1
2363; Sphingobium chlorophenolicum L-1 chromosome 1,
complete sequence; 334100279; CP002798.1
2364; Streptomyces violaceusniger Tu 4113, complete genome;
345007964; NC_015957.1
2365; Rhodospirillum rubrum F11, complete genome; 386348020;
NC_017584.1
2366; Actinoplanes sp. SE50/110, complete genome;
386845069; NC_017803.1
2367; Emticicia oligotrophica DSM 17448, complete
genome; 408671769; NC_018748.1
2368; Tistrella mobilis KA081020-065 plasmid pTM1, complete
sequence; 442559580; NC_017957.2
2369; Bacillus thuringiensis MC28, complete genome;
407703236; NC_018693.1
2370; Nostoc sp. PCC 7107, complete genome; 427705465;
NC_019676.1
2371; Synechococcus sp. PCC 6312, complete genome;
427711179; NC_019680.1
2372; Stanieria cyanosphaera PCC 7437, complete
genome; 428267688; CP003653.1
2373; Desulfocapsa sulfexigens DSM 10523, complete
genome; 451945650; NC_020304.1
2374; Xanthomonas citri pv. mangiferaeindicae LMG 941, whole
genome shotgun sequence; 381169556; NZ_CAHO01000002.1
2375; Streptomyces fulvissimus DSM 40593, complete
genome; 488607535; NC_021177.1
2376; Streptomyces rapamycinicus NRRL 5491 genome;
521353217; CP006567.1
2377; Gloeobacter kilaueensis JS1, complete genome;
554634310; NC_022600.1
2378; Kutzneria albida DSM 43870, complete genome;
754862786; NZ_CP007155.1
2379; Mesorhizobium huakuii 7653R genome; 657121522;
CP006581.1
2380; Burkholderia thailandensis E264 chromosome I,
complete sequence; 83718394; NC_007651.1
2381; Sphingopyxis fiibergensis strain Kp5.2, complete
genome; 749188513; NZ_CP009122.1
2382; Sphingopyxis fiibergensis strain Kp5.2, complete
genome; 749188513; NZ_CP009122.1
2383; Streptomyces sp. ZJ306 hydroxylase, deacetylase, and
hypothetical proteins genes, complete cds; ikarugamycin gene
cluster, complete sequence; and GCN5-related N-acetyltransferase,
hypothetical protein, asparagine synthase, transcriptional
regulator, ABC transporter, hypothetical proteins, putative
membrane transport protein, putative acetyltransferase,
cytochrome P450, putative alpha-glucosidase, phosphoketolase,
helix-turn-helix domain-containing protein,
membrane protein, NAD-dependent epimera; 746616581;
KF954512.1
2384; Streptomyces albus strain DSM 41398, complete genome;
749658562; NZ_CP010519.1
2385; Amycolatopsis lurida NRRL 2430, complete genome;
755908329; CP007219.1
2386; Streptomyces lydicus A02, complete genome; 822214995;
NZ_CP007699.1
2387; Streptomyces lydicus A02, complete genome; 822214995;
NZ_CP007699.1
2388; Streptomyces lydicus A02, complete genome; 822214995;
NZ_CP007699.1
2389; Streptomyces xiamenensis strain 318, complete genome;
921170702; NZ_CP009922.2
2390; Streptomyces xiamenensis strain 318, complete genome;
921170702; NZ_CP009922.2
2391; Uncultured bacterium clone AZ25P121 genomic sequence;
818476494; KP274854.1
2392; Streptomyces sp. PBH53 genome; 852460626; CP011799.1
2393; Streptomyces sp. PBH53 genome; 852460626; CP011799.1
2394; Streptomyces sp. PBH53 genome; 852460626; CP011799.1
2395; Sphingopyxis sp. 113P3, complete genome; 924898949;
NZ_CP009452.1
2396; Sphingopyxis sp. 113P3, complete genome; 924898949;
NZ_CP009452.1
2397; Bifidobacterium longum subsp infantis strain BT1,
complete genome; 927296881; CP010411.1
2398; Nostoc piscinale CENA21 genome; 930349143; CP012036.1
2399; Citromicrobium sp. JL477, complete genome; 932136007;
CP011344.1
2400; Sphingopyxis macrogoltabida strain 203, complete genome;
938956730; NZ_CP009429.1
2401; Sphingopyxis macrogoltabida strain 203 plasmid,
complete sequence; 938956814; NZ_CP009430.1
2402; Paenibacillus sp. 32O-W, complete genome; 961447255;
CP013653.1
2403; Streptomyces avermitilis MA-4680 = NBRC 14893,
complete genome; 162960844; NC_003155.4
2404; Streptomyces avermitilis MA-4680 = NBRC 14893,
complete genome; 162960844; NC_003155.4
2405; Kitasatospora setae KM-6054 DNA, complete genome;
357386972; NC_016109.1
2406; Rhodococcus jostii lariatin biosynthetic gene cluster (larA,
larB, larC, larD, larE), complete cds; 380356103dbjAB593691.1; 0
2407; Rubrivivax gelatinosus IL144 DNA, complete genome;
383755859; NC_017075.1
2408; Pseudomonas sp. Os17 DNA, complete genome;
771839907dbjAP014627.1; 0
2409; Pseudomonas sp. St29 DNA, complete genome;
771846103dbjAP014628.1; 0
2410; Fischerella sp. NIES-3754 DNA, complete genome;
965684975dbjAP017305.1; 0
2411; Magnetospirillum gryphiswaldense MSR-1 v2, complete
genome; 568144401; NC_023065.1
2412; Magnetospirillum gryphiswaldense MSR-1 v2, complete
genome; 568144401; NC_023065.1
2413; Streptococcus suis SC84 complete genome, strain SC84;
253750923; NC_012924.1
2414; Salinibacter ruber M8 chromosome, complete genome;
294505815; NC_014032.1
2415; Enterococcus faecalis ATCC 29212 contig24, whole
genome shotgun sequence; 401673929; ALOD01000024.1
2416; Saccharothrix espanaensis DSM 44229 complete genome;
433601838; NC_019673.1
2417; Roseburia sp. CAG: 197 WGS project CBBL01000000
data, contig, whole genome shotgun sequence; 524261006;
CBBL010000225.1
2418; Roseburia sp. CAG: 197 WGS project CBBL01000000
data, contig, whole genome shotgun sequence; 524261006;
CBBL010000225.1
2419; Clostridium sp. CAG: 221 WGS project CBDC01000000
data, contig, whole genome shotgun sequence; 524362382;
CBDC010000065.1
2420; Clostridium sp. CAG: 411 WGS project CBIY01000000 data,
contig, whole genome shotgun sequence; 524742306;
CBIY010000075.1
2421; Roseburia sp. CAG: 100 WGS project CBKV01000000 data,
contig, whole genome shotgun sequence; 524842500;
CBKV010000277.1
2422; Novosphingobium sp. KN65.2 WGS project CCBH000000000
data, contig SPHy1_Contig_228, whole genome shotgun sequence;
808402906; CCBH010000144.1
2423; Mesorhizobium plurifarium genome assembly Mesorhizobium
plurifarium ORS1032T genome assembly, contig MPL1032_Contig_21,
whole genome shotgun sequence; 927916006; CCND01000014.1
2424; Kibdelosporangium sp. MJ126-NF4, whole genome shotgun
sequence; 754819815; NZ_CDME01000002.1
2425; Kibdelosporangium sp. MJ126-NF4 genome assembly High
quaKibdelosporangium sp. MJ126-NF4, scaffold BPA_8, whole
genome shotgun sequence; 747653426; CDME01000011.1
2426; Methanobacterium formicicum genome assembly isolate
Mb9, chromosome: I; 952971377; LN734822.1
2427; Streptococcus pneumoniae strain 37, whole genome shotgun
sequence; 912648153; NZ_CKHR01000004.1
2428; Streptococcus pneumoniae genome assembly 6631_3#4,
scaffold ERS019570SCcontig000005, whole genome shotgun
sequence; 879201007; CKIK01000005.1
2429; Streptococcus pneumoniae strain type strain: N, whole
genome shotgun sequence; 950938054; NZ_CIHL01000007.1
2430; Streptococcus pneumoniae strain 37, whole genome
shotgun sequence; 912648153; NZ_CKHR01000004.1
2431; Klebsiella variicola genome assembly Kv4880, contig
BN1200_Contig_75, whole genome shotgun sequence; 906292938;
CXPB01000073.1
2432; Klebsiella variicola genome assembly KvT29A, contig
BN1200_Contig_98, whole genome shotgun sequence; 906304012;
CXPA01000125.1
2433; Bacillus cereus genome assembly Bacillus JRS4, contig
contig000025, whole genome shotgun sequence; 924092470;
CYHM01000025.1
2434; Achromobacter sp. 27895TDY5663426 genome assembly,
contig. ERS372662SCcontig000003, whole genome shotgun
sequence; 928675838; CYTQ01000003.1
2435; Pedobacter sp. BAL39 1103467000492, whole genome
shotgun sequence; 149277373; NZ_ABCM01000005.1
2436; Streptomyces sp. Mg1 supercont1.100, whole genome
shotgun sequence; 254387191; NZ_D5570483.1
2437; Streptomyces sviceus ATCC 29083 chromosome, whole
genome shotgun sequence; 297196766; NZ_CM000951.1
2438; Streptomyces pristinaespiralis ATCC 25486 chromosome,
whole genome shotgun sequence; 297189896; NZ_CM000950.1
2439; Enterococcus faecalis ATCC 4200 supercont1.2, whole
genome shotgun sequence; 239948580; NZ_GG670372.1
2440; Enterococcus faecalis ATCC 29212 contig24, whole genome
shotgun sequence; 401673929; ALOD01000024.1
2441; Streptomyces roseosporus NRRL 15998 supercont3.1 genomic
scaffold, whole genome shotgun sequence; 221717172; D5999644.1
2442; Streptococcus vestibularis F0396 ctg1126932565723, whole
genome shotgun sequence; 311100538; AEKO01000007.1
2443; Streptococcus vestibularis F0396 ctg1126932565723, whole
genome shotgun sequence; 311100538; AEKO01000007.1
2444; Ruminococcus albus 8 contig00035, whole genome shotgun
sequence; 325680876; NZ_ADKM02000123.1
2445; Streptomyces sp. W007 contig00293, whole genome shotgun
sequence; 365867746; NZ_AGSW01000272.1
2446; Streptomyces sp. W007 contig00241, whole genome shotgun
sequence; 365866490; NZ_AGSW01000226.1
2447; Burkholderia pseudomallei 1258a Contig0089, whole genome
shotgun sequence; 418540998; NZ_AHJB01000089.1
2448; Burkholderia pseudomallei 1026a Contig0036, whole genome
shotgun sequence; 385360120; AHJA01000036.1
2449; Rhodanobacter sp. 115 contig437, whole genome shotgun
sequence; 389759651; NZ_AJXS01000437.1
2450; Rhodanobacter thiooxydans LCS2 contig057, whole
genome shotgun sequence; 389809081; NZ_AJXW01000057.1
2451; Burkholderia thailandensis MSMB43 Scaffold3, whole
genome shotgun sequence; 424903876; NZ_JH692063.1
2452; Streptomyces auratus AGR0001 Scaffold1, whole
genome shotgun sequence; 398790069; NZ_JH725387.1
2453; Actinomyces naeslundii str. Howell 279 ctg1130888818142,
whole genome shotgun sequence; 399903251; ALJK01000024.1
2454; Enterococcus faecalis ATCC 29212 contig24, whole
genome shotgun sequence; 401673929; ALOD01000024.1
2455; Uncultured bacterium ACD_75C02634, whole genome
shotgun sequence; 406886663; AMFJ01033303.1
2456; Amycolatopsis decaplanina DSM 44594 Contig0055,
whole genome shotgun sequence; 458848256; NZ_AOHO01000055.1
2457; Streptomyces mobaraensis NBRC 13819 = DSM 40847
contig024, whole genome shotgun sequence; 458977979;
NZ_AORZ01000024.1
2458; Burkholderia pseudomallei MSHR1043 seq0003, whole
genome shotgun sequence; 469643984; AOGU01000003.1
2459; Enterococcus faecalis EnGen0363 strain RMC5 acAqY-
supercont1.4, whole genome shotgun sequence; 502232520;
NZ_KB944632.1
2460; Enterococcus faecalis EnGen0233 strain UAA1014 acvJV-
supercont1.10.C18, whole genome shotgun sequence; 487281881;
AIZW01000018.1
2461; Pandoraea sp. SD6-2 scaffold29, whole genome shotgun
sequence; 505733815; NZ_KB944444.1
2462; Streptomyces aurantiacus JA 4570 Seq28, whole genome
shotgun sequence; 514916412; NZ_AOPZ01000028.1
2463; Streptomyces aurantiacus JA 4570 Seq17, whole genome
shotgun sequence; 514916021; NZ_AOPZ01000017.1
2464; Enterococcus faecalis LA3B-2 Scaffold22, whole genome
shotgun sequence; 522837181; NZ_KE352807.1
2465; Paenibacillus alvei A6-6i-x PAAL66ix 14, whole genome
shotgun sequence; 528200987; ATMS01000061.1
2466; Dehalobacter sp. UNSWDHB Contig_139, whole genome
shotgun sequence; 544905305; NZ_AUUR01000139.1
2467; Actinobaculum sp. oral taxon 183 str. F0552 Scaffold15,
whole genome shotgun sequence; 545327527; NZ_KE951412.1
2468; Actinobaculum sp. oral taxon 183 str. F0552 Scaffold1,
whole genome shotgun sequence; 545327174; NZ_KE951406.1
2469; Propionibacterium acidifaciens F0233 ctg1127964738299,
whole genome shotgun sequence; 544249812; ACVN02000045.1
2470; Rubidibacter lacunae KORDI 51-2 KR5 l_contig00121,
whole genome shotgun sequence; 550281965; NZ_ASSJ01000070.1
2471; Rothia aeria F0184 R aeriaHMPREF0742-1.0_Cont136.4,
whole genome shotgun sequence; 551695014; AXZG01000035.1
2472; Candidatus Halobonum tyrrellensis G22 contig00002,
whole genome shotgun sequence; 557371823; NZ_ASGZ01000002.1
2473; Streptomyces niveus NCIMB 11891 chromosome, whole
genome shotgun sequence; 566146291; NZ_CM002280.1
2474; Blastomonas sp. CACIA14H2 contig00049, whole
genome shotgun sequence; 563282524; AYSC01000019.1
2475; Frankia sp. CcI6 CcI6DRAFT_scaffold_51.52, whole
genome shotgun sequence; 563312125; AYTZ01000052.1
2476; Frankia sp. CcI6 CcI6DRAFT_scaffold_16.17, whole
genome shotgun sequence; 564016690; NZ_AYTZ01000017.1
2477; Clostridium butyricum DORA_1 Q607_CBUC00058,
whole genome shotgun sequence; 566226100; AZLX01000058.1
2478; Streptococcus sp. DORA_10 Q617_5P5C00257, whole
genome shotgun sequence; 566231608; AZMH01000257.1
2479; Candidatus Entotheonella factor TSY1_contig00913, whole
genome shotgun sequence; 575408569; AZHW01000959.1
2480; Candidatus Entotheonellagemina TSY2_contig00559,
whole genome shotgun sequence; 575423213; AZHX01000559.1
2481; Streptomyces roseosporus NRRL 11379 supercont4.1, whole
genome shotgun sequence; 588273405; NZ_ABYX02000001.1
2482; Frankia sp. Thr ThrDRAFT_scaffold_48.49, whole
genome shotgun sequence; 602261491; JENI01000049.1
2483; Frankia sp. CcI6 CcI6DRAFT_scaffold_51.52, whole genome
shotgun sequence; 563312125; AYTZ01000052.1
2484; Frankia sp. Thr ThrDRAFT_scaffold_28.29, whole genome
shotgun sequence; 602262270; JENI01000029.1
2485; Novosphingobium resinovorum strain KF1 contig000008,
whole genome shotgun sequence; 738615271; NZ_JFYZ01000008.1
2486; Novosphingobium resinovorum strain KF1 contig000008,
whole genome shotgun sequence; 738615271; NZ_JFYZ01000008.1
2487; Brevundimonas abyssalis TAR-001 DNA, contig: BAB005,
whole genome shotgun sequence; 543418148dbjBATC01000005.1; 0
2488; Bacillus akibai JCM 9157, whole genome shotgun sequence;
737696658; NZ_BAUV01000025.1
2489; Bacillus akibai JCM 9157, whole genome shotgun sequence;
737696658; NZ_BAUV01000025.1
2490; Bacillus boroniphilus JCM 21738 DNA, contig: contig_6,
whole genome shotgun sequence; 571146044dbjBAUW01000006.1; 0
2491; Bacillus sp. 17376 scaffold00002, whole genome shotgun
sequence; 560433869; NZ_K1547189.1
2492; Gracilibacillus boraciitolerans JCM 21714 DNA,
contig: contig_30, whole genome shotgun sequence;
575082509dbjBAVS01000030.1; 0
2493; Gracilibacillus boraciitolerans JCM 21714 DNA,
contig: contig_30, whole genome shotgun sequence;
575082509dbjBAVS01000030.1; 0
2494; Bacterium endosymbiont of Mortierella elongata FMR23-6,
whole genome shotgun sequence; 779889750; NZ_DF850521.1
2495; Sphingopyxis sp. C-1 DNA, contig: contig_1, whole genome
shotgun sequence; 834156795dbjBBRO01000001.1; 0
2496; Sphingopyxis sp. C-1 DNA, contig: contig_1, whole genome
shotgun sequence; 834156795dbjBBRO01000001.1; 0
2497; Sphingopyxis sp. C-1 DNA, contig: contig_1, whole genome
shotgun sequence; 834156795dbjBBRO01000001.1; 0
2498; Ideonella sakaiensis strain 201-F6, whole genome shotgun
sequence; 928998724; NZ_BBYR01000007.1
2499; Brevundimonas sp. EAKA contig5, whole genome shotgun
sequence; 737322991; NZ_JMQR01000005.1
2500; Streptomyces griseorubens strain JSD-1 scaffold1, whole
genome shotgun sequence; 739792456; NZ_KL503830.1
2501; Frankia sp. Thr ThrDRAFT_scaffold 28.29, whole
genome shotgun sequence; 602262270; JENI01000029.1
2502; Frankia sp. Allo2 ALLO2DRAFT scaffold 25.26, whole
genome shotgun sequence; 737764929; NZ_JPHT01000026.1
2503; Frankia sp. CcI6 CcI6DRAFT_scaffold_16.17, whole
genome shotgun sequence; 564016690; NZ_AYTZ01000017.1
2504; Bifidobacterium reuteri DSM 23975 Contig04, whole
genome shotgun sequence; 672991374; JGZK01000004.1
2505; Streptomyces sp. JS01 contig2, whole genome
shotgun sequence; 695871554; NZ_JPWW01000002.1
2506; Sphingopyxis sp. LC81 contig28, whole genome
shotgun sequence; 686470905; JNFD01000021.1
2507; Sphingopyxis sp. LC81 contig24, whole genome
shotgun sequence; 739659070; NZ_JNFD01000017.1
2508; Sphingopyxis sp. LC363 contig36, whole genome
shotgun sequence; 739702045; NZ_JNFC01000030.1
2509; Burkholderia pseudomallei strain BEF DP42.Contig323,
whole genome shotgun sequence; 686949962; JPNR01000131.1
2510; Xanthomonas cannabis pv. phaseoli strain Nyagatare scf_52938_
7, whole 0 genome shotgun sequence; 835885587; NZ_KN265462.1
2511; Burkholderia pseudomallei MSHR1000 scaffold1, whole
genome shotgun 0 sequence; 740963677; NZ_KN323065.1
2512; Burkholderia pseudomallei M5HR435 Y033. Contig530,
whole genome shotgun sequence; 715120018; JRFP01000024.1
2513; Candidatus Thiomargarita nelsonii isolate Hydrate Ridge contig
1164, whole genome shotgun sequence; 723288710; JSZA01001164.1
2514; Paenibacillus sp. P1XP2 CM49_contig000046, whole
genome shotgun sequence; 727078508; JRNV01000046.1
2515; Novosphingobium sp. P6W scaffold9, whole genome
shotgun sequence; 763095630; NZ_JXZE01000009.1
2516; Streptomyces griseus strain S4-7 contig113, whole genome
shotgun sequence; 764464761; NZ_JYBE01000113.1
2517; Lechevalieria aerocolonigenes strain NRRL B-16140 contig11.3,
whole genome shotgun sequence; 772744565; NZ_JYJG01000059.1
2518; Desulfobulbaceae bacterium BRH_c16a BRHa_1001515,
whole genome shotgun sequence; 780791108; LADS01000058.1
2519; Peptococcaceae bacterium BRH_c4b BRHa_1001357,
whole genome shotgun sequence; 780813318; LADO01000010.1
2520; Peptococcaceae bacterium BRH_c4b BRHa_1001357,
whole genome shotgun sequence; 780813318; LADO01000010.1
2521; Hyphomonadaceae bacterium BRH_c29 BRHa_1005676,
whole genome shotgun sequence; 780821511; LADW01000068.1
2522; Hyphomonas sp. BRH_c22 BRHa_1001979, whole
sequence; 780834515; LADU01000087.1
2523; Streptomyces rubellomurinus subsp. indigoferus strain
ATCC 31304 contig-55, whole genome shotgun sequence;
783374270; NZ_JZKG01000056.1
2524; Streptomyces sp. NRRL S-444 contig322.4, whole
genome shotgun sequence; 797049078; JZWX01001028.1
2525; Streptomyces sp. NRRL B-1568 contig-76, whole
genome shotgun sequence; 799161588; NZ_JZWZ01000076.1
2526; Candidate division TM6 bacterium GW2011_GWF2_36_131
US03_C0013, whole genome shotgun sequence; 818310996;
LBRK01000013.1
2527; Sphingobium czechense LL01 25410_1, whole genome
shotgun sequence; 861972513; JACT01000001.1
2528; Streptomyces caatingaensis strain CMAA 1322 contig02,
whole genome shotgun sequence; 906344334; NZ_LFXA01000002.1
2529; Erythrobacter citreus LAMA 915 Contig13, whole genome
shotgun sequence; 914607448; NZ_JYNE01000028.1
2530; Paenibacillus polymyxa strain YUPP-8 scaffold32, whole
genome shotgun sequence; 924434005; LIYK01000027.1
2531; Burkholderia mallei GB8 horse 4 contig_394, whole genome
shotgun sequence; 67639376; NZ_AAHO01000116.1
2532; Streptomyces rimosus subsp. rimosus strain NRRL WC-3909
P217contig95.1, whole genome shotgun sequence; 925286515;
LGCO01000284.1
2533; Streptomyces rimosus subsp. rimosus strain NRRL WC-3909
P217contig56.1, whole genome shotgun sequence; 925291008;
LGCO01000241.1
2534; Streptomyces rimosus subsp. rimosus strain NRRL WC-3869
P248contig50.1, whole genome shotgun sequence; 925315417;
LGCQ01000244.1
2535; Streptomyces rimosus subsp. rimosus strain NRRL WC-3869
P248contig20.1, whole genome shotgun sequence; 925322461;
LGCQ01000113.1
2536; Streptomyces rimosus subsp. rimosus strain NRRL WC-3898
P259contig86.1, whole genome shotgun sequence; 927279089;
BRHa_1005676, whole genome NZ_LGCU01000353.1
2537; Streptomyces rimosus subsp. pseudoverticillatus strain
NRRL WC-3896 genome shotgun P270contig8.1, whole genome
shotgun sequence; 927292684; NZ_LGCV01000415.1
2538; Streptomyces rimosus subsp. pseudoverticillatus strain
NRRL WC-3896 P270contig51.1, whole genome shotgun sequence;
927292651; NZ_LGCV01000382.1
2539; Streptomyces sp. NRRL F-5755 P309contig7.1, whole
genome shotgun sequence; 926371541; NZ_LGCW01000295.1
2540; Streptomyces sp. NRRL F-5755 P309contig50.1, whole
genome shotgun sequence; 926371520; NZ_LGCW01000274.1
2541; Streptomyces sp. NRRL F-5755 P309contig48.1, whole
genome shotgun sequence; 926371517; NZ_LGCW01000271.1
2542; Streptomyces sp. NRRL F-6492 P446contig3.1, whole
genome shotgun sequence; 926315769; NZ_LGEG01000211.1
2543; Streptomyces sp. XY332 P409contig34.1, whole genome
shotgun sequence; 927093145; NZ_LGHN01000166.1
2544; Novosphingobium sp. ST904 contig_104, whole genome
shotgun sequence; 935540718; NZ_LGJH01000063.1
2545; Actinobacteria bacterium OK006 ctg96, whole genome
shotgun sequence; 930491003; NZ_LJCU01000287.1
2546; Actinobacteria bacterium OK074 ctg60, whole genome
shotgun sequence; 930473294; NZ_LJCV01000275.1
2547; Betaproteobacteria bacterium SG8_39_WOR_8-12_2589,
whole genome shotgun sequence; 931421682; LJTQ01000030.1
2548; Candidate division BRC1 bacterium SM23_51 WORSMTZ_
10094, whole genome shotgun sequence; 931536013; LJUL01000022.1
2549; Bacillus vietnamensis strain UCD-SED5 scaffold_15, whole
genome shotgun sequence; 933903534; LIXZ01000017.1
2550; Xanthomonas arboricola strain CITA 44 CITA_44_contig_26,
whole genome shotgun sequence; 937505789; NZ_LJGM01000026.1
2551; Xanthomonas sp. Mitacek01 contig_17, whole genome
shotgun sequence; 941965142; NZ_LKIT01000002.1
2552; Erythrobacteraceae bacterium HL-111 ITZY_scaf_51,
whole genome shotgun sequence; 938259025; LJSW01000006.1
2553; Halomonas sp. HL-93 ITZY_scaf_415, whole genome
shotgun sequence; 938285459; LJST01000237.1
2554; Paenibacillus sp. Soil724D2 contig_11, whole genome
shotgun sequence; 946400391; LMRY01000003.1
2555; Leucobacter sp. G161 contig50, whole genome shotgun
sequence; 970293907; LOHP01000076.1
2556; Streptomyces silvensis strain ATCC 53525 53525_Assembly_
Contig_22, whole genome shotgun sequence; 970361514;
LOCL01000028.1
2557; Streptococcus pneumoniae 2071004 gspj3.contig.3,
whole genome shotgun sequence; 421236283; NZ_ALBJ01000004.1
2558; Streptococcus pneumoniae 70585, complete genome;
225857809; NC_012468.1
2559; Bacillus cereus R309803 chromosome, whole genome
shotgun sequence; 238801472; NZ_CM000720.1
2560; Bacillus cereus AH1271 chromosome, whole genome
shotgun sequence; 238801491; NZ_CM000739.1
2561; Bacillus thuringiensis serovar andalousiensis BGSC 4AW1
chromosome, whole genome shotgun sequence; 238801506;
NZ_CM000754.1
2562; Bacillus cereus VD115 supercont1.1, whole genome
shotgun sequence; 423614674; NZ_JH792165.1
2563; Bacillus cereus Rock4-18 chromosome, whole genome
shotgun sequence; 238801487; NZ_CM000735.1
2564; Bacillus cereus Rock1-3 chromosome, whole genome
shotgun sequence; 238801480; NZ_CM000728.1
2565; Bacillus cereus Rock3-29 chromosome, whole genome
shotgun sequence; 238801483; NZ_CM000731.1
2566; Bacillus cereus VD148 supercont1.1, whole genome shotgun
sequence; 423621402; NZ_JH792156.1
2567; Bacillus thuringiensis MC28, complete genome; 407703236;
NC_018693.1
2568; Bacillus cereus BAG5X2-1 supercont1.1, whole
genome shotgun sequence; 423456860; NZ_JH791975.1
2569; Bacillus cereus BAG3X2-1 supercont1.1, whole genome
shotgun sequence; 423416528; NZ_JH791923.1
2570; Bacillus cereus BAG1X1-3 supercont1.1, whole genome
shotgun sequence; 423388152; NZ_JH792182.1
2571; Escherichia coli KTE150 acwoI-supercont1.4, whole
genome shotgun sequence; 433109554; NZ_ANYF01000004.1
2572; Bacillus cereus NVH0597-99 gcontig2_1106483384196,
whole genome shotgun sequence; 196038187; NZ_ABDK02000003.1
2573; Bacillus cereus AH621 chromosome, whole genome
shotgun sequence; 238801471; NZ_CM000719.1
2574; Bacillus cereus AH603 chromosome, whole genome
shotgun sequence; 238801489; NZ_CM000737.1
2575; Bacillus cereus VD142 actaa-supercont2.2, whole
genome shotgun sequence; 514340871; NZ_KE150045.1
2576; Bacillus cereus BAG6O-2 supercont1.1, whole genome
shotgun sequence; 423468694; NZ_JH804628.1
2577; Bacillus cereus BtB2-4 supercont1.1, whole genome
shotgun sequence; 423485377; NZ_JH804642.1
2578; Bacillus cereus HuA2-1 supercont1.1, whole genome
shotgun sequence; 423508503; NZ_JH804672.1
2579; Bacillus cereus HuA4-10 supercont1.1, whole genome
shotgun sequence; 423520617; NZ_JH792148.1
2580; Bacillus cereus MC67 supercont1.2, whole genome
shotgun sequence; 423557538; NZ_JH792114.1
2581; Bacillus cereus VD078 supercont1.1, whole genome
shotgun sequence; 423597198; NZ_JH792251.1
2582; Bacillus cereus VD107 supercont1.1, whole genome
shotgun sequence; 423609285; NZ_JH792232.1
2583; Bacillus mycoides DSM 2048 chromosome, whole genome
shotgun sequence; 238801494; NZ_CM000742.1
2584; Bacillus cereus VDM034 supercont1.1, whole genome
shotgun sequence; 423666303; NZ_JH791809.1
2585; Bacillus cereus BAG5X1-1 supercont1.1, whole genome
shotgun sequence; 423451256; NZ_JH791996.1
2586; Enterococcus faecalis ATCC 29212 contig24, whole
genome shotgun sequence; 401673929; ALOD01000024.1
2587; Enterococcus faecalis TX1341 Scfld578, whole genome
shotgun sequence; 422736691; NZ_GL457197.1
2588; Clostridium butyricum 60E.3 actYk-supercont1.1, whole
genome shotgun sequence; 488644557; NZ_KB851128.1
2589; Rhodobacter sphaeroides WS8N chromosome chrI, whole
genome shotgun sequence; 332561612; NZ_CM001161.1
2590; Microcystis aeruginosa PCC 9807, whole genome
shotgun sequence; 425454132; NZ_HE973326.1
2591; Brevundimonas diminuta ATCC 11568 BDIM_scaffold00005,
whole genome shotgun sequence; 329889017; NZ_GL883086.1
2592; Brevundimonas diminuta 470-4 Scfld7, whole genome
shotgun sequence; 444405902; NZ_KB291784.1
2593; Bacillus mycoides Rock1-4 chromosome, whole genome
shotgun sequence; 238801495; NZ_CM000743.1
2594; Clostridium butyricum 5521 gcontig_1106103650482, whole
genome shotgun sequence; 182420360; NZ_ABDT01000120.2
2595; Xanthomonas citri pv. mangiferaeindicae LMG 941, whole
genome shotgun sequence; 381169556; NZ_CAHO01000002.1
2596; Xanthomonas citri pv. mangiferaeindicae LMG 941, whole
genome shotgun sequence; 381171950; NZ_CAHO01000029.1
2597; Methylosinus trichosporium OB3b MettrDRAFT_Contig106_C,
whole genome shotgun sequence; 639846426; NZ_ADVE02000001.1
2598; Streptomyces clavuligerus ATCC 27064 supercont1.55,
whole genome shotgun sequence; 254392242; NZ_DS570678.1
2599; Streptomyces rimosus subsp. rimosus strain NRRL WC-3909
P217contig95.1, whole genome shotgun sequence; 925286515;
LGCO01000284.1
2600; Streptomyces rimosus subsp. rimosus strain NRRL WC-3909
P217contig56.1, whole genome shotgun sequence; 925291008;
LGCO01000241.1
2601; Streptomyces viridochromogenes DSM 40736 supercont1.1,
whole genome shotgun sequence; 224581107; NZ_GG657757.1
2602; Streptomyces viridochromogenes DSM 40736 supercont1.1,
whole genome shotgun sequence; 224581107; NZ_GG657757.1
2603; Streptomyces viridochromogenes Tue57 Seq127, whole
genome shotgun sequence; 443625867; NZ_AMLP01000127.1
2604; Methanobacterium formicicum DSM 3637 Contig04, whole
genome shotgun sequence; 408381849; NZ_AMPO01000004.1
2605; Burkholderia pseudomallei MSHR435 Y033. Contig530,
whole genome shotgun sequence; 715120018; JRFP01000024.1
2606; Burkholderia mallei GB8 horse 4 contig_394, whole
genome shotgun sequence; 67639376; NZ_AAHO01000116.1
2607; Sphingobium yanoikuyae ATCC 51230 supercont1.1,
whole genome shotgun sequence; 427407324; NZ_H4992904.1
2608; Sphingobium yanoikuyae ATCC 51230 supercont1.1,
whole genome shotgun sequence; 427407324; NZ_JH992904.1
2609; Sphingobium yanoikuyae ATCC 51230 supercont1.1,
whole genome shotgun sequence; 427407324; NZ_JH992904.1
2610; Burkholderia pseudomallei MSHR1043 seq0003, whole
genome shotgun sequence; 469643984; AOGU01000003.1
2611; Burkholderia pseudomallei strain BEF DP42. Contig323,
whole genome shotgun sequence; 686949962; JPNR01000131.1
2612; Burkholderia pseudomallei S13 scf_1041068450778, whole
shotgun sequence; 254197184; NZ_CH899773.1
genome 2613; Burkholderia pseudomallei 1026a Contig0036, whole
genome shotgun sequence; 385360120; AHJA01000036.1
2614; Burkholderia pseudomallei 305 g_contig_BUA. Contig1097,
whole genome shotgun sequence; 134282186; NZ_AAYX01000011.1
2615; Burkholderia pseudomallei 576 BUC. Contig184, whole
genome shotgun sequence; 217421258; NZ_ACCE01000004.1
2616; [Eubacterium] cellulosolvens 6 chromosome, whole genome
shotgun sequence; 389575461; NZ_CM001487.1
2617; Amycolatopsis azurea DSM 43854 contig60, whole genome
shotgun sequence; 451338568; NZ_ANMG01000060.1
2618; Xanthomonas axonopodis pv. malvacearum str. GSPB1386
1386_Scaffold6, whole genome shotgun sequence; 418516056;
NZ_AHIB01000006.1
2619; Xanthomonas citti pv. punicae str. LMG 859, whole genome
shotgun sequence; 390991205; NZ_CAGJ01000031.1
2620; Bacillus pseudomycoides DSM 12442 chromosome, whole
genome shotgun sequence; 238801497; NZ_CM000745.1
2621; Mesorhizobium amorphae CCNWGS0123 contig00204, whole
genome shotgun sequence; 357028583; NZ_AGSN01000187.1
2622; Xanthomonas gardneri ATCC 19865 XANTHO7DRAF_
Contig52, whole genome shotgun sequence; 325923334;
NZ_AEQX01000392.1
2623; Xenococcus sp. PCC 7305 scaffold_00124, whole genome
shotgun sequence; 443325429; NZ_ALVZ01000124.1
2624; Leptolyngbya sp. PCC 7375 Lepto7375DRAFT_LPA.5,
whole genome shotgun sequence; 427415532; NZ_M993797.1
2625; Streptomyces auratus AGR0001 Scaffold1, whole genome
shotgun sequence; 398790069; NZ_JH725387.1
2626; Paenibacillus dendritiformis C454 PDENDC1000064, whole
genome shotgun sequence; 374605177; NZ_AHKH01000064.1
2627; Halosimplex carlsbadense 2-9-1 contig_4, whole genome
shotgun sequence; 448406329; NZ_AOIU01000004.1
2628; Rothia aeria F0474 contig00003, whole genome shotgun
sequence; 383809261; NZ_AJJQ01000036.1
2629; Paenibacillus lactis 154 ctg179, whole genome shotgun
sequence; 354585485; NZ_AGIP01000020.1
2630; Fictibacillus macauensis ZFHKF-1 Contig20, whole genome
shotgun sequence; 392955666; NZ_AKKV01000020.1
2631; Marine gamma proteobacterium HTCC2148 scf_1106774214169,
whole genome shotgun sequence; 254480798; NZ_DS999224.1
2632; Paenibacillus sp. Aloe-11 GW8_15, whole genome
shotgun sequence; 375307420; NZ_JH601049.1
2633; Rhodanobacter denitrificans strain 116-2 contig032, whole
genome shotgun sequence; 389798210; NZ_AJXV01000032.1
2634; Frankia saprophytica strain CN3 FrCN3DRAFT_FCB.2, whole
genome shotgun sequence; 652876473; NZ_KI912267.1
2635; Caulobacter sp. AP07 PMI01_contig_53.53, whole genome
shotgun sequence; 399069941; NZ_AKKF01000033.1
2636; Novosphingobium sp. AP12 PMI02_contig_78.78, whole
genome shotgun sequence; 399058618; NZ_AKKE01000021.1
2637; Sphingobium sp. AP49 PMI04_contig490.490, whole
genome shotgun sequence; 398386476; NZ_AJVL01000086.1
2638; Desulfosporosinus youngiae DSM 17734 chromosome,
whole genome shotgun sequence; 374578721; NZ_CM001441.1
2639; Moorea producens 3L scf52054, whole genome shotgun
sequence; 332710503; NZ_GL890955.1
2640; Pedobacter sp. BAL39 1103467000500, whole genome
shotgun sequence; 149277003; NZ_ABCM01000004.1
2641; Sulfurovum sp. AR contig00449, whole genome shotgun
sequence; 386284588; NZ_AJLE01000006.1
2642; Mucilaginibacter paludis DSM 18603 chromosome, whole
genome shotgun sequence; 373951708; NZ_CM001403.1
2643; Mucilaginibacter paludis DSM 18603 chromosome, whole
genome shotgun sequence; 373951708; NZ_CM001403.1
2644; Magnetospirillum caucaseum strain SO-1 contig00006, whole
genome shotgun sequence; 458904467; NZ_AONQ01000006.1
2645; Sphingomonas sp. LH128 Contig3, whole genome
shotgun sequence; 402821166; NZ_ALVC01000003.1
2646; Sphingomonas sp. LH128 Contig8, whole genome
shotgun sequence; 402821307; NZ_ALVC01000008.1
2647; Novosphingobium sp. Rr 2-17 contig98, whole genome
shotgun sequence; 393773868; NZ_AKFJ01000097.1
2648; Streptomyces sp. AA4 supercont1.3, whole genome
shotgun sequence; 224581098; NZ_GG657748.1
2649; Moorea producens 3L scf52052, whole genome shotgun
sequence; 332710285; NZ_GL890953.1
2650; Cecembia lonarensis LW9 contig000133, whole genome
shotgun sequence; 406663945; NZ_AMGM01000133.1
2651; Actinomyces sp. oral taxon 848 str. F0332 Scfld0, whole
genome shotgun sequence; 260447107; NZ_GG703879.1
2652; Actinomyces sp. oral taxon 848 str. F0332 Scfld0, whole
genome shotgun sequence; 260447107; NZ_GG703879.1
2653; Streptomyces ipomoeae 91-03 gcontig_1108499710267, whole
genome shotgun sequence; 429195484; NZ_AEJC01000118.1
2654; Frankia sp. QA3 chromosome, whole genome
shotgun sequence; 392941286; NZ_CM001489.1
2655; Fischerella sp. JSC-11 ctg112, whole genome
shotgun sequence; 354566316; NZ_AGIZ01000005.1
2656; Rhodobacter sp. AKP1 contig19, whole genome
shotgun sequence; 429208285; NZ_ANFS01000019.1
2657; Sphingomonas sp. SKA58 scf_1100007010440, whole
genome shotgun sequence; 211594417; NZ_CH959308.1
2658; Rubfivivax benzoatilyticus JA2 = ATCC BAA-35 strain
JA2 contig_155, whole genome shotgun sequence; 332527785;
NZ_AEWG01000155.1
2659; Streptomyces clavuligerus ATCC 27064 plasmid pSCL3,
whole genome shotgun sequence; 326336949; NZ_CM001018.1
2660; Streptomyces chartreusis NRRL 12338 12338_Doro1_scaffold19,
whole genome shotgun sequence; 381200190; NZ_JH164855.1
2661; Candidatus Odyssella thessalonicensis L13 HMO_scaffold00016,
whole genome shotgun sequence; 343957487; NZ_AEWF01000005.1
2662; Candidatus Odyssella thessalonicensis L13 HMO_scaffold00016,
whole genome shotgun sequence; 343957487; NZ_AEWF01000005.1
2663; Sphingobium yanoikuyae XLDN2-5 contig000022, whole
genome shotgun sequence; 378759068; NZ_AFXE01000022.1
2664; Sphingobium yanoikuyae XLDN2-5 contig000029, whole
genome shotgun sequence; 378759075; NZ_AFXE01000029.1
2665; Paenibacillus peofiae KCTC 3763 contig9, whole
genome shotgun sequence; 389822526; NZ_AGFX01000048.1
2666; Citromicrobium sp. JLT1363 contig00009, whole
genome shotgun sequence; 341575924; NZ_AEUE01000009.1
2667; [Pseudomonas] geniculata N1 contig35, whole genome
shotgun sequence; 921165904; NZ_AJLO02000014.1
2668; Pseudomonas extremaustralis 14-3 substr. 14-3b strain
14-3 contig00001, whole genome shotgun sequence;
394743069; NZ_AHIP01000001.1
2669; Streptomyces sp. S4, whole genome shotgun sequence;
358468594; NZ_FR873693.1
2670; Streptomyces sp. S4, whole genome shotgun sequence;
358468601; NZ_FR873700.1
2671; Bacillus timonensis strain MM10403188, whole genome
shotgun sequence; 403048279; NZ_HE610988.1
2672; Lunatimonas lonarensis strain AK24 S14_contig_18, whole
genome shotgun sequence; 499123840; NZ_AQHR01000021.1
2673; Mesorhizobium loti MAFF303099 DNA, complete
genome; 57165207; NC_002678.2
2674; Legionella pneumophila subsp. pneumophila ATCC
43290, complete genome; 378775961; NC_016811.1
2675; Xanthomonas axonopodis pv. citfi str. 306, complete
genome; 21240774; NC_003919.1
2676; Thermobifida fusca YX, complete genome; 72160406;
NC_007333.1
2677; Rhodobacter sphaeroides 2.4.1 chromosome 1, whole
genome shotgun sequence; 482849861; NZ_AKBU01000001.1
2678; Rhodospirillum rubrum F11, complete genome;
386348020; NC_017584.1
2679; Rhodospirillum rubrum F11, complete genome;
386348020; NC_017584.1
2680; Rhodospirillum rubrum F11, complete genome;
386348020; NC_017584.1
2681; Hahella chejuensis KCTC 2396, complete genome;
83642913; NC_007645.1
2682; Frankia sp. Thr ThrDRAFT_scaffold_48.49, whole
genome shotgun sequence; 602261491; JENI01000049.1
2683; Frankia sp. Thr ThrDRAFT_scaffold_28.29, whole
genome shotgun sequence; 602262270; JENI01000029.1
2684; Novosphingobium aromaticivorans DSM 12444,
complete genome; 87198026; NC_007794.1
2685; Roseobacter denitfificans OCh 114, complete
genome; 110677421; NC_008209.1
2686; Frankia alni str. ACN14A chromosome, complete
sequence; 111219505; NC_008278.1
2687; Pelobacter propionicus DSM 2379, complete genome;
118578449; NC_008609.1
2688; Psychromonas ingrahamii 37, complete genome;
119943794; NC_008709.1
2689; Rhodobacter sphaeroides ATCC 17029 chromosome 1,
complete sequence; 126460778; NC_009049.1
2690; Burkholdefia pseudomallei 668 chromosome I,
complete sequence; 126438353; NC_009074.1
2691; Rhodobacter sphaeroides ATCC 17025, complete
genome; 146276058; NC_009428.1
2692; Geobacter uraniireducens Rf4, complete genome;
148262085; NC_009483.1
2693; Sulfurovum sp. NBC37-1 genomic DNA, complete
genome; 152991597; NC_009663.1
2694; Acaryochloris marina MBIC11017, complete
genome; 158333233; NC_009925.1
2695; Bacillus weihenstephanensis KBAB4, complete
genome; 163938013; NC_010184.1
2696; Caulobacter sp. K31 plasmid pCAUL01, complete
sequence; 167621728; NC_010335.1
2697; Caulobacter sp. K31, complete genome; 167643973;
NC_010338.1
2698; Candidatus Amoebophilus asiaticus 5a2, complete
genome; 189501470; NC_010830.1
2699; Stenotrophomonas maltophilia R551-3, complete
genome; 194363778; NC_011071.1
2700; Bifidobacterium longum subsp infantis ATCC 15697,
complete genome; 213690928; NC_011593.1
2701; Cyanothece sp. PCC 7425, complete genome; 220905643;
NC_011884.1
2702; Chitinophaga pinensis DSM 2588, complete genome;
256419057; NC_013132.1
2703; Haliangium ochraceum DSM 14365, complete genome;
262193326; NC_013440.1
2704; Rhodothermus marinus DSM 4252, complete genome;
268315578; NC_013501.1
2705; Thermobaculum terrenum ATCC BAA-798 chromosome
1, complete sequence; 269925123; NC_013525.1
2706; Thermobaculum terrenum ATCC BAA-798 chromosome
2, complete sequence; 269838913; NC_013526.1
2707; Thermobaculum terrenum ATCC BAA-798 chromosome
2, complete sequence; 269838913; NC_013526.1
2708; Sphingobium japonicum UT26S DNA, chromosome 1,
complete genome; 294009986; NC_014006.1
2709; Sphingobium japonicum UT26S plasmid pCHQ1 DNA,
complete genome; 294023656; NC_014007.1
2710; Salinibacter ruber M8 chromosome, complete
genome; 294505815; NC_014032.1
2711; Salinibacter ruber M8 chromosome, complete
genome; 294505815; NC_014032.1
2712; Legionella pneumophila 2300/99 Alcoy, complete
genome; 296105497; NC_014125.1
2713; Nocardiopsis dassonvillei subsp. dassonvillei DSM 43111
chromosome 1, complete sequence; 297558985; NC_014210.1
2714; Amycolatopsis mediten-anei S699, complete
genome; 384145136; NC_017186.1
2715; Butyrivibrio proteoclasticus B316 chromosome 1,
complete sequence; 302669374; NC_014387.1
2716; Paenibacillus polymyxa E681, complete genome;
864439741; NC_014483.2
2717; Paenibacillus polymyxa M1 main chromosome,
complete genome; 386038690; NC_017542.1
2718; Leadbetterella byssophila DSM 17132, complete
genome; 312128809; NC_014655.1
2719; Frankia inefficax, complete genome; 312193897;
NC_014666.1
2720; Frankia inefficax, complete genome; 312193897;
NC_014666.1
2721; Burkholderia rhizoxinica HKI 454, complete
genome; 312794749; NC_014722.1
2722; Burkholderia rhizoxinica HKI 454, complete
genome; 312794749; NC_014722.1
2723; Asticcacaulis excentricus CB 48 chromosome 2,
complete sequence; 315499382; NC_014817.1
2724; Teniglobus saanensis SP1PR4, complete genome;
320105246; NC_014963.1
2725; Syntrophobotulus glycolicus DSM 8271, complete
genome; 325288201; NC_015172.1
2726; Methanobacterium lacus strain AL-21, complete
genome; 325957759; NC_015216.1
2727; Marinomonas mediterranea MMB-1, complete
genome; 326793322; NC_014006.1 NC_015276.1
2728; Desulfobacca acetoxidans DSM 11109, complete
genome; 328951746; NC_015388.1
2729; Methylomonas methanica MC09, complete genome;
333981747; NC_015572.1
2730; Methylomonas methanica MC09, complete genome;
333981747;NC_015572.1
2731; Methanobacterium paludis strain SWAN1, complete
genome; 333986242; NC_015574.1
2732; Novosphingobium sp. PP1Y Lpl large plasmid,
complete replicon; 334133217; NC_015579.1
2733; Novosphingobium sp. PP1Y main chromosome,
complete replicon; 334139601; NC_015580.1
2734; Frankia symbiont of Datisca glomerata, complete
genome; 336176139; NC_015656.1
2735; Halopiger xanaduensis SH-6 plasmid pHALXA01,
complete genome; 336251750; NC_015658.1
2736; Mesorhizobium opportunistum WSM2075, complete
genome; 337264537; NC_015675.1
2737; Runella slithyformis DSM 19594, complete genome;
338209545; NC_015703.1
2738; Runella slithyformis DSM 19594, complete genome;
338209545; NC_015703.1
2739; Roseobacter litoralis Och 149, complete genome;
339501577; NC_015730.1
2740; Streptomyces violaceusniger Tu 4113 plasmid pSTRVI01,
complete sequence; 345007457; NC_015951.1
2741; Rhodothennus marinus SG0.5JP17-172, complete genome;
345301888; NC_015966.1
2742; Sphingobium sp. SYK-6 DNA, complete genome;
347526385; NC_015976.1
2743; Sphingobium sp. SYK-6 DNA, complete genome;
347526385; NC_015976.1
2744; Chloracidobacterium thermophilum B chromosome 1,
complete sequence; 347753732; NC_016024.1
2745; Kitasatospora setae KM-6054 DNA, complete genome;
357386972; NC_016109.1
2746; Kitasatospora setae KM-6054 DNA, complete genome;
357386972; NC_016109.1
2747; Streptomyces cattleya str. NRRL 8057 main chromosome,
complete genome; 357397620; NC_016111.1
2748; Desulfosporosinus orientis DSM 765, complete genome;
374992780; NC_016584.1
2749; Paenibacillus tenae HPL-003, complete genome;
374319880; NC_016641.1
2750; Bacillus megaterium WSH-002, complete genome;
384044176; NC_017138.1
2751; Francisella cf. novicida 3523, complete genome;
387823583; NC_017449.1
2752; Streptococcus salivarius JIM8777 complete genome;
387783149; NC_017595.1
2753; Tistrella mobilis KA081020-065, complete genome;
389875858; NC_017956.1
2754; Tistrella mobilis KA081020-065 plasmid pTM3,
complete sequence; 389874236; NC_017958.1
2755; Legionella pneumophila subsp. pneumophila str. Lorraine
chromosome, complete genome; 397662556; NC_018139.1
2756; Nocardiopsis alba ATCC BAA-2165, complete
genome; 403507510; NC_018524.1
2757; Streptomyces venezuelae ATCC 10712 complete
genome; 408675720; NC_018750.1
2758; Saccharothrix espanaensis DSM 44229 complete
genome; 433601838; NC_019673.1
2759; Nostoc sp. PCC 7107, complete genome;
427705465; NC_019676.1
2760; Rivularia sp. PCC 7116, complete genome;
427733619; NC_019678.1
2761; Rivularia sp. PCC 7116, complete genome;
427733619; NC_019678.1
2762; Synechococcus sp. PCC 6312, complete genome;
427711179; NC_019680.1
2763; Nostoc sp. PCC 7524, complete genome;
427727289; NC_019684.1
2764; Calothrix sp. PCC 6303, complete genome;
428296779; NC_019751.1
2765; Crinalium epipsammum PCC 9333, complete
genome; 428303693; NC_019753.1
2766; Cylindrospermum stagnale PCC 7417, complete
genome; 434402184; NC_019757.1
2767; Thermobacillus composti KWC4, complete genome;
430748349; NC_019897.1
2768; Mesorhizobium australicum WSM2073, complete
genome; 433771415; NC_019973.1
2769; Rhodanobacter denitrificans strain 2APBS1, complete
genome; 469816339; NC_020541.1
2770; Bacillus sp. 1NLA3E, complete genome; 488570484;
NC_021171.1
2771; Bacillus sp. 1NLA3E, complete genome; 488570484;
NC_021171.1
2772; Burkholdefia thailandensis MSMB121 chromosome 1,
complete sequence; 488601775; NC_021173.1
2773; Streptomyces davawensis strain JCM 4913 complete
genome; 471319476; NC_020504.1
2774; Streptomyces davawensis strain JCM 4913 complete
genome; 471319476; NC_020504.1
2775; Desulfotomaculum acetoxidans DSM 771, complete
genome; 258513366; NC_013216.1
2776; Desulfotomaculum acetoxidans DSM 771, complete
genome; 258513366; NC_013216.1
2777; Actinosynnema mirum DSM 43827, complete genome;
256374160; NC_013093.1
2778; Actinosynnema mirum DSM 43827, complete genome;
256374160; NC_013093.1
2779; Rhodobacter sphaeroides KD131 chromosome 1,
complete sequence; 221638099; NC_011963.1
2780; Bacillus cereus BAG2O-3 acfXF-supercont1.1, whole
genome shotgun sequence; 507017505; NZ_KB976530.1
2781; Bacillus cereus HuA2-9 acqVt-supercont1.1, whole
genome shotgun sequence; 507020427; NZ_KB976152.1
2782; Bacillus cereus HuA3-9 acqVv-supercont1.4, whole
genome shotgun sequence; 507024338; NZ_KB976146.1
2783; Bacillus cereus VD118 acrHo-supercont1.9, whole
genome shotgun sequence; 507035131; NZ_KB976800.1
2784; Bacillus cereus VD131 acrHi-supercont1.9, whole
genome shotgun sequence; 507037581; NZ_KB976660.1
2785; Bacillus cereus VD136 acrHc-supercont1.1, whole
genome shotgun sequence; 507041177; NZ_KB976717.1
2786; Bacillus cereus VDM019 achrj-supercont1.2, whole
genome shotgun sequence; 507056808; NZ_KB976199.1
2787; Bacillus cereus VDM053 acrGS-supercont1.7, whole
genome shotgun sequence; 507060152; NZ_KB976714.1
2788; Bacillus cereus VDM006 acrHb-supercont1.1, whole
genome shotgun sequence; 507060269; NZ_KB976864.1
2789; Bacillus cereus VDM021 acrHe-supercont1.1, whole
genome shotgun sequence; 507061629; NZ_KB976905.1
2790; Thermobifida fusca TM51 contig028, whole genome
shotgun sequence; 510814910; NZ_AOSG01000028.1
2791; Halomonas anticafiensis FP35 = DSM 16096 strain FP35
Scaffold1, whole genome shotgun sequence; 514429123;
NZ_KE332377.1
2792; Halomonas anticafiensis FP35 = DSM 16096 strain FP35
Scaffold1, whole genome shotgun sequence; 514429123;
NZ_KE332377.1
2793; Halomonas anticafiensis FP35 = DSM 16096 strain FP35
Scaffold1, whole genome shotgun sequence; 514429123;
NZ_KE332377.1
2794; Streptomyces sp. HPH0547 aczHZ-supercont1.2, whole
genome shotgun sequence; 512676856; NZ_KE150472.1
2795; Acinetobacter gyllenbergii MTCC 11365 contig1, whole
genome shotgun sequence; 514348304; NZ_ASQH01000001.1
2796; Streptomyces aurantiacus JA 4570 Seq63, whole genome
shotgun sequence; 514917321; NZ_AOPZ01000063.1
2797; Streptomyces aurantiacus JA 4570 Seq109, whole
genome shotgun sequence; 514918665; NZ_AOPZ01000109.1
2798; Actinoalloteichus spitiensis RMV-1378 Contig406, whole
genome shotgun sequence; 483112234; NZ_AGVX02000406.1
2799; Paenibacillus polymyxa OSY-DF Contig136, whole
genome shotgun sequence; 484036841; NZ_AIPP01000136.1
2800; Fischerella muscicola SAG 1427-1 = PCC 73103 contig00215,
whole genome shotgun sequence; 484073367; NZ_AJLJ01000207.1
2801; Fischerella muscicola PCC 7414 contig00109, whole
genome shotgun sequence; 484075173; NZ_AJLK01000109.1
2802; Fischerella muscicola PCC 7414 contig00153, whole
genome shotgun sequence; 484075372; NZ_AJLK01000153.1
2803; Fischerella thermalis PCC 7521 contig00099, whole
genome shotgun sequence; 484076371; NZ_AJLL01000098.1
2804; Xanthomonas arboficola pv. juglandis str. NCPPB 1447
contig00105, whole genome shotgun sequence; 484083029;
NZ_AJTL01000105.1
2805; Sphingobium xenophagum QYY contig015, whole
genome shotgun sequence; 484272664; NZ_AKM01000015.1
2806; Pedobacter arcticus A12 Scaffold2, whole genome
shotgun sequence; 484345004; NZ_JH947126.1
2807; Leptolyngbya boryana PCC 6306 LepboDRAFT_LPC.1,
whole genome shotgun sequence; 482909028; NZ_KB731324.1
2808; Spirulina subsalsa PCC 9445 Contig210, whole genome
shotgun sequence; 482909235; NZ_JH980292.1
2809; Fischerella sp. PCC 9339 PCC9339DRAFT_scaffold1.1,
whole genome shotgun sequence; 482909394; NZ_JH992898.1
2810; Mastigocladopsis repens PCC 10914 Mas10914DRAFT_
scaffold1.1, whole genome shotgun sequence; 482909462;
NZ_JH992901.1
2811; Methylowccus capsulatus str. Texas = ATCC 19069 strain
Texas contig0129, whole genome shotgun sequence;
483090991; NZ_AMCE01000064.1
2812; Lactococcus garvieae Tac2 Tac2Contig_33, whole genome
shotgun sequence; 483258918; NZ_AMFE01000033.1
2813; Paenisporosarcina sp. TG-14 111.TG14.1_1, whole
genome shotgun sequence; 483299154; NZ_AMGD01000001.1
2814; Paenibacillus sp. ICGEB2008 Contig_7, whole genome
shotgun sequence; 483624383; NZ_AMQU01000007.1
2815; Amphibacillus jilinensis Y1 Scaffold2, whole genome
shotgun sequence; 483992405; NZ_JH976435.1
2816; Alpha proteobacterium LLX12A LLX12A_contig00014,
whole genome shotgun sequence; 483996931; NZ_AMYX01000014.1
2817; Alpha proteobacterium LLX12A LLX12A_contig00026,
whole genome shotgun sequence; 483996974; NZ_AMYX01000026.1
2818; Alpha proteobacterium LLX12A LLX12A_contig00084,
whole genome shotgun sequence; 483997176; NZ_AMYX01000084.1
2819; Alpha proteobacterium LA1A L41A_contig00002, whole
genome shotgun sequence; 483997957; NZ_AMYY01000002.1
2820; Nocardiopsis alba DSM 43377 contig 10, whole genome
shotgun sequence; 484007121; NZ_ANAC01000010.1
2821; Nocardiopsis sp. TP-A0876 strain NBRC 110039, whole
genome shotgun sequence; 754924215; NZ_BAZE01000001.1
2822; Nocardiopsis halophila DSM 44494 contig_138, whole
genome shotgun sequence; 484007841; NZ_ANAD01000138.1
2823; Nocardiopsis halophila DSM 44494 contig_138, whole
genome shotgun sequence; 484007841; NZ_ANAD01000138.1
2824; Nocardiopsis halophila DSM 44494 contig_197, whole
genome shotgun sequence; 484008051; NZ_ANAD01000197.1
2825; Nocardiopsis baichengensis YIM 90130 Scaffold15_1, whole
genome shotgun sequence; 484012558; NZ_ANAS01000033.1
2826; Nocardiopsis halotolerans DSM 44410 contig_26, whole
genome shotgun sequence; 484015294; NZ_ANAX01000026.1
2827; Nocardiopsis kunsanensis DSM 44524 contig_3, whole
genome shotgun sequence; 484016825; NZ_ANAY01000003.1
2828; Nocardiopsis kunsanensis DSM 44524 contig_16, whole
genome shotgun sequence; 484016872; NZ_ANAY01000016.1
2829; Nocardiopsis potens DSM 45234 contig_25, whole
genome shotgun sequence; 484017897; NZ_ANBB01000025.1
2830; Nocardiopsis lucentensis DSM 44048 contig_935, whole
genome shotgun sequence; 484021665; NZ_ANBC01000935.1
2831; Nocardiopsis alkaliphila YIM 80379 contig_111, whole
genome shotgun sequence; 484022237; NZ_ANBD01000111.1
2832; Nocardiopsis sauna YIM 90010 contig_87, whole genome
shotgun sequence; 484023389; NZ_ANBF01000087.1
2833; Nocardiopsis sauna YIM 90010 contig_204, whole genome
shotgun sequence; 484023808; NZ_ANBF01000204.1
2834; Nocardiopsis chromatogenes YIM 90109 contig_59, whole
genome shotgun sequence; 484026076; NZ_ANBH01000059.1
2835; Porphyrobacter sp. AAP82 Contig35, whole genome
shotgun sequence; 484033307; NZ_ANFX01000035.1
2836; Blastomonas sp. AAP53 Contig8, whole genome shotgun
sequence; 484033611; NZ_ANFZ01000008.1
2837; Blastomonas sp. AAP53 Contig14, whole genome shotgun
sequence; 484033631; NZ_ANFZ01000014.1
2838; Paenibacillus sp. PAMC 26794 5104_29, whole genome
shotgun sequence; 484070054; NZ_ANHX01000029.1
2839; Oscillatoria sp. PCC 10802 Osc10802DRAFT_Contig7.7,
whole genome shotgun sequence; 484104632; NZ_KB235948.1
2840; Oscillatoria sp. PCC 10802 Osc10802DRAFT_Contig7.7,
whole genome shotgun sequence; 484104632; NZ_KB235948.1
2841; Clostridium botulinum CB11/1-1 CB_contig00105, whole
genome shotgun sequence; 484141779; NZ_AORM01000006.1
2842; Actinopolyspora halophila DSM 43834 ActhaDRAFT_
contig1.1_C, whole genome shotgun sequence; 484203522;
NZ_AQUI01000002.1
2843; Asticcacaulis benevestitus DSM 16100 = ATCC BAA-896
strain DSM 16100 B060DRAFT_scaffold_12.13_C, whole genome
shotgun sequence; 484226753; NZ_AQWM01000013.1
2844; Asticcacaulis benevestitus DSM 16100 = ATCC BAA-896
strain DSM 16100 B060DRAFT_scaffold_31.32_C, whole genome
shotgun sequence; 484226810; NZ_AQWM01000032.1
2845; Streptomyces sp. FxanaC1 B074DRAFT_scaffold_1.2_C,
whole genome shotgun sequence; 484227180; NZ_AQW001000002.1
2846; Streptomyces sp. FxanaC1 B074DRAFT_scaffold_7.8_C,
whole genome shotgun sequence; 484227195; NZ_AQW001000008.1
2847; Smaragdicoccus niigatensis DSM 44881 = NBRC 103563
strain DSM 44881 F600DRAFT_scaffold00011.11_C, whole genome
shotgun sequence; 484234624; NZ_AQXZ01000009.1
2848; Sphingomonas melonis DAPP-PG 224 Sphme3DRAFT_
scaffold1.1, whole genome shotgun sequence; 482984722;
NZ_KB900605.1
2849; Verrucomicrobium sp. 3C A37ADRAFT_scaffold1.1,
whole genome shotgun sequence; 483219562; NZ_KB901875.1
2850; Verrucomicrobium sp. 3C A37ADRAFT_scaffold1.1,
whole genome shotgun sequence; 483219562; NZ_KB901875.1
2851; Bradyrhizobium sp. WSM2793 A3ASDRAFT_scaffold
genome shotgun sequence; 483314733; NZ_KB902785.1
2852; Streptomyces vitaminophilus DSM 41686 A3IGDRAFT_
scaffold_10.11, whole genome shotgun sequence; 483682977;
NZ_KB904636.1
2853; Ancylobacter sp. FA202 A3M1DRAFT_scaffold1.1, whole
genome shotgun sequence; 483720774; NZ_KB904818.1
2854; Filamentous cyanobactenum ESFC-1 A3MYDRAFT_
scaffold1.1, whole genome shotgun sequence; 483724571;
NZ_KB904821.1
2855; Streptomyces sp. CcaIMP-8W B053DRAFT_scaffold_17.18,
whole genome shotgun sequence; 483961830; NZ_KB890924.1
2856; Streptomyces sp. ScaeMP-e10 B061DRAFT_scaffold_01,
whole genome shotgun sequence; 483967534; NZ_KB891296.1
2857; Streptomyces sp. KhCrAH-244 B069DRAFT_scaffold_11.12,
whole genome shotgun sequence; 483969755; NZ_KB891596.1
2858; Streptomyces sp. HmicA12 B072DRAFT_scaffold_19.20,
whole genome shotgun sequence; 483972948; NZ_KB891808.1
2859; Streptomyces sp. MspMP-M5 B073DRAFT_scaffold 27.28,
whole genome shotgun sequence; 483974021; NZ_KB891893.1
2860; Arthrobacter sp. 161MFSha2.1 C567DRAFT_scaffold00006.6,
whole genome shotgun sequence; 484021228; NZ_KB895788.1
2861; Streptomyces sp. CNY228 D330DRAFT_scaffold00011.11,
whole genome shotgun sequence; 484057944; NZ_KB898231.1
2862; Streptomyces sp. CNB091 D581DRAFT_scaffold00010.10,
whole genome shotgun sequence; 484070161; NZ_KB898999.1
2863; Sphingobium xenophagum NBRC 107872, whole genome
shotgun sequence; 483527356; NZ_BARE01000016.1
2864; Streptomyces sp. TOR3209 Contig612, whole genome shotgun
sequence; 484867900; NZ_AGNH01000612.1
2865; Streptomyces sp. TOR3209 Contig613, whole genome shotgun
sequence; 484867902; NZ_AGNH01000613.1
2866; Stenotrophomonas maltophilia RR-10 STMALcontig40,
whole genome shotgun sequence; 484978121; NZ_AGRB01000040.1
2867; Bacillus oceanisediminis 2691 contig2644, whole genome
shotgun sequence; 485048843; NZ_ALEG01000067.1
2868; Calothrix sp. PCC 7103 Cal7103DRAFT_CPM.6, whole
genome shotgun 24.25, whole sequence; 485067373; NZ_KB217478.1
2869; Pseudanabaena sp. PCC 6802 Pse6802_scaffold_5, whole
genome shotgun sequence; 485067426; NZ_KB235914.1
2870; Actinomadura atramentaiia DSM 43919 strain SF2197
G339DRAFT_scaffold00002.2, whole genome shotgun sequence;
485090585; NZ_KB907209.1
2871; Novispirillum itersonii subsp. itersonii ATCC 12639
G365DRAFT_scaffold00001.1, whole genome shotgun sequence;
485091510; NZ_KB907337.1
2872; Novispirillum itersonii subsp. itersonii ATCC 12639
G365DRAFT_scaffold00001.1, whole genome shotgun sequence;
485091510; NZ_KB907337.1
2873; Paenibacillus polymyxa ATCC 842 PPt02_scaffold1,
whole genome shotgun sequence; 485269841; NZ_GL905390.1
2874; Actinopolyspora mortivallis DSM 44261 strain HS-1
ActmoDRAFT_scaffold1.1, whole genome shotgun sequence;
486324513; NZ_KB913024.1
2875; Mesorhizobium loti NZP2037 Meslo3DRAFT_scaffold1.1,
whole genome shotgun sequence; 486325193; NZ_KB913026.1
2876; Paenibacillus sp. HW567 B212DRAFT_scaffold1.1, whole
genome shotgun sequence; 486346141; NZ_KB910518.1
2877; Bacillus sp. 123MFChir2 H280DRAFT_scaffold00030.30,
whole genome shotgun sequence; 487368297; NZ_KB910953.1
2878; Streptomyces canus 299MFChir4.1 H293DRAFT_
scaffold00032.32, whole genome shotgun sequence; 487385965;
NZ_KB911613.1
2879; Kribbella catacumbae DSM 19601 A3ESDRAFT_scaffold_
7.8_C, whole genome shotgun sequence; 484207511;
NZ_AQUZ01000008.1
2880; Paenibacillus riograndensis SBR5 Contig78, whole genome
shotgun sequence; 485470216; NZ_A
2881; Lamprocystis purpurea DSM 4197 A39ODRAFT_scaffold_0.1,
whole genome shotgun sequence; 483254584; NZ_KB902362.1
2882; Nonomumea coxensis DSM 45129 A3G7DRAFT_scaffold_4.5,
whole genome shotgun sequence; 483454700; NZ_KB903974.1
2883; Streptomyces scabrisporus DSM 41855 A3ICDRAFT_scaffold_01,
whole genome shotgun sequence; 483624586; NZ_KB889561.1
2884; Amycolatopsis alba DSM 44262 scaffold1, whole genome shotgun
sequence; 486330103; NZ_KB913032.1
2885; Amycolatopsis benzoatilytica AK 16/65 AmybeDRAFT_scaffold1.1,
whole genome shotgun sequence; 486399859; NZ_KB912942.1
2886; Amycolatopsis nigrescens CSC17Ta-90 AmyniDRAFT_Contig68.1_
C, whole genome shotgun sequence; 487404592; NZ_ARVW01000001.1
2887; Amycolatopsis nigrescens CSC17Ta-90 AmyniDRAFT_Contig68.1_
C, whole genome shotgun sequence; 487404592; NZ_ARVW01000001.1
2888; Amycolatopsis nigrescens CSC17Ta-90 AmyniDRAFT_Contig68.1_
C, whole genome shotgun sequence; 487404592; NZ_ARVW01000001.1
2889; Reyranella massiliensis 521, whole genome shotgun
sequence; 484038067; NZ_HE997181.1
2890; Acidobacteriaceae bacterium KBS 83 G002DRAFT_
scaffold00007.7, whole genome shotgun sequence; 485076323;
NZ_KB906739.1
2891; Sphingobium lactosutens DS20 contig107, whole genome
shotgun sequence; 544811486; NZ_ATDP01000107.1
2892; Novosphingobium lindaniclasticum LE124 contig147,
whole genome shotgun sequence; 544819688; NZ_ATHL01000147.1
2893; Actinobaculum sp. oral taxon 183 str. F0552 Scaffold15,
whole genome shotgun sequence; 545327527; NZ_KE951412.1
2894; Novosphingobium sp. B-7 scaffold147, whole genome shotgun
sequence; 514419386; NZ_KE148338.1
2895; Sphingomonas-like bacterium B12, whole genome shotgun
sequence; 484113405; NZ_BACX01000237.1
2896; Sphingomonas-like bacterium B12, whole genome shotgun
sequence; 484113491; NZ_BACX01000258.1
2897; Thermoactinomyces vulgaris strain NRRL F-5595 F5595contig15.1,
whole genome shotgun sequence; 929862756; NZ_LGKI01000090.1
2898; Clostridium saccharobutylicum DSM 13864, complete genome;
550916528; NC_022571.1
2899; Butyrivibrio fibrisolvens AB2020 G616DRAFT_scaffold00015.15_
C, whole genome shotgun sequence; 551012921; NZ_ATVZ01000015.1
2900; Butyrivibrio sp. XPD2006 G590DRAFT_scaffold00008.8_C, whole
whole genome shotgun sequence; 551021553; NZ_ATVT01000008.1
2901; Butyrivibrio sp. AE3009 G588DRAFT_scaffold00030.30_C,
whole genome shotgun sequence; 551035505; NZ_ATVS01000030.1
2902; Acidobacteriaceae bacterium TAA166 strain TAA 166
H979DRAFT_scaffold_0.1_C, whole genome shotgun sequence;
551216990; NZ_ATWD01000001.1
2903; Acidobacteriaceae bacterium TAA166 strain TAA 166
H979DRAFT_scaffold_0.1_C, whole genome shotgun sequence;
551216990; NZ_ATWD01000001.1
2904; Acidobacteriaceae bacterium TAA166 strain TAA 166
H979DRAFT_scaffold_0. l_C, whole genome shotgun sequence;
551216990; NZ_ATWD01000001.1
2905; Leptolyngbya sp. Heron Island J 50, whole genome
shotgun sequence; 553739852; NZ_AWNH01000066.1
2906; Leptolyngbya sp. Heron Island J 50, whole genome
shotgun sequence; 553739852; NZ_AWNH01000066.1
2907; Leptolyngbya sp. Heron Island J 67, whole genome
shotgun sequence; 553740975; NZ_AWNH01000084.1
2908; Klebsiella pneumoniae BIDMC 22 addSE-supercont1.4,
whole genome shotgun sequence; 556268595; NZ_KI535436.1
2909; Klebsiella pneumoniae MGH 19 addTc-supercont1.2,
whole genome shotgun sequence; 556494858; NZ_KI535678.1
2910; Asticcacaulis sp. AC466 contig00008, whole genome
shotgun sequence; 557833377; NZ_AWGE01000008.1
2911; Asticcacaulis sp. AC466 contig00033, whole genome
shotgun sequence; 557835508; NZ_AWGE01000033.1
2912; Asticcacaulis sp. YBE204 contig00005, whole genome
shotgun sequence; 557839256; NZ_AWGF01000005.1
2913; Asticcacaulis sp. YBE204 contig00010, whole genome
shotgun sequence; 557839714; NZ_AWGF01000010.1
2914; Streptomyces roseochromogenus subsp. oscitans DS 12.976
chromosome, whole genome shotgun sequence; 566155502;
NZ_CM002285.1
2915; Streptomyces roseochromogenus subsp. oscitans DS 12.976
chromosome, whole genome shotgun sequence; 566155502;
NZ_CM002285.1
2916; Bacillus sp. 17376 scaffold00002, whole genome shotgun
sequence; 560433869; NZ_KI547189.1
2917; Mesorhizobium sp. LSJC285A00 scaffold0007, whole
genome shotgun sequence; 563442031; NZ_AYVK01000007.1
2918; Mesorhizobium sp. LSJC277A00 scaffold0014, whole
genome shotgun sequence; 563459186; NZ_AYVM01000014.1
2919; Mesorhizobium sp. LSJC269B00 scaffold0015, whole
genome shotgun sequence; 563464990; NZ_AYVN01000015.1
2920; Mesorhizobium sp. LSJC268A00 scaffold0012, whole
genome shotgun sequence; 563469252; NZ_AYVO01000012.1
2921; Mesorhizobium sp. LSJC265A00 scaffold0015, whole
genome shotgun sequence; 563472037; NZ_AYVP01000015.1
2922; Mesorhizobium sp. LSJC264A00 scaffold0029, whole
genome shotgun sequence; 563478461; NZ_AYVQ01000029.1
2923; Mesorhizobium sp. LSJC255A00 scaffold0001, whole
genome shotgun sequence; 563480247; NZ_AYVR01000001.1
2924; Mesorhizobium sp. LSHC426A00 scaffold0005, whole
genome shotgun sequence; 563492715; NZ_AYVV01000005.1
2925; Mesorhizobium sp. LSHC422A00 scaffold0012, whole
genome shotgun sequence; 563497640; NZ_AYVX01000012.1
2926; Mesorhizobium sp. LNJC405B00 scaffold0005, whole
genome shotgun sequence; 563523441; NZ_AYWC01000005.1
2927; Mesorhizobium sp. LNJC403B00 scaffold0001, whole
genome shotgun sequence; 563526426; NZ_AYWD01000001.1
2928; Mesorhizobium sp. LNJC399B00 scaffold0004, whole
genome shotgun sequence; 563530011; NZ_AYWE01000004.1
2929; Mesorhizobium sp. LNJC398B00 scaffold0002, whole
genome shotgun sequence; 563532486; NZ_AYWF01000002.1
2930; Mesorhizobium sp. LNJC395A00 scaffold0011, whole
genome shotgun sequence; 563536456; NZ_AYWG01000011.1
2931; Mesorhizobium sp. LNJC394B00 scaffold0005, whole
genome shotgun sequence; 563539234; NZ_AYWH01000005.1
2932; Mesorhizobium sp. LNJC384A00 scaffold0009, whole
genome shotgun sequence; 563544477; NZ_AYWK01000009.1
2933; Mesorhizobium sp. LNJC380A00 scaffold0009, whole
genome shotgun sequence; 563546593; NZ_AYWL01000009.1
2934; Mesorhizobium sp. LNHC232B00 scaffold0020, whole
genome shotgun sequence; 563561985; NZ_AYWP01000020.1
2935; Mesorhizobium sp. LNHC229A00 scaffold0006, whole
genome shotgun sequence; 563567190; NZ_AYWQ01000006.1
2936; Mesorhizobium sp. LNHC221B00 scaffold0001, whole
genome shotgun sequence; 563570867; NZ_AYWR01000001.1
2937; Mesorhizobium sp. LNHC220B00 scaffold0002, whole
genome shotgun sequence; 563576979; NZ_AYWS01000002.1
2938; Mesorhizobium sp. LNHC209A00 scaffold0002, whole
genome shotgun sequence; 563784877; NZ_AYWT01000002.1
2939; Mesorhizobium sp. L48C026A00 scaffold0030, whole
genome shotgun sequence; 563848676; NZ_AYWU01000030.1
2940; Mesorhizobium sp. L2C089B000 scaffold0011, whole
genome shotgun sequence; 563888034; NZ_AYWV01000011.1
2941; Mesorhizobium sp. L2C084A000 scaffold0007, whole
genome shotgun sequence; 563938926; NZ_AYWX01000007.1
2942; Mesorhizobium sp. L2C067A000 scaffold0014, whole
genome shotgun sequence; 563977521; NZ_AYWY01000014.1
2943; Mesorhizobium sp. L2C066B000 scaffold0012, whole
genome shotgun sequence; 563993080; NZ_AYWZ01000012.1
2944; Mesorhizobium sp. L103C119B0 scaffold0005, whole
genome shotgun sequence; 564005047; NZ_AYXE01000005.1
2945; Mesorhizobium sp. L103C105A0 scaffold0004, whole
genome shotgun sequence; 564008267; NZ_AYXF01000004.1
2946; Xanthomonas hortorum pv. carotae str. M081 chromosome,
whole genome shotgun sequence; 565808720; NZ_CM002307.1
2947; Clostridium pasteurianum NRRL B-598, complete genome;
930593557; NZ_CP011966.1
2948; Paenibacillus polymyxa CR1, complete genome;
734699963; NC_023037.2
2949; Streptococcus suis SC84 complete genome, strain
SC84; 253750923; NC_012924.1
2950; Streptococcus suis 10581 Contig00069, whole genome
shotgun sequence; 636868927; NZ_ALKQ01000069.1
2951; Burkholderia pseudomallei HBPUB10134a BP_10134a_103,
whole genome shotgun sequence; 638832186; NZ_AVAL01000102.1
2952; Mycobacterium sp. UM_WGJ Contig_32, whole genome
shotgun sequence; 638971293; NZ_AUWR01000032.1
2953; Mycobacterium iranicum UM_TJL Contig_42, whole genome
shotgun sequence; 638987534; NZ_AUWT01000042.1
2954; Mesorhizobium ciceri CMG6 MescicDRAFT_scaffold_1.2_C,
whole genome shotgun sequence; 639162053; NZ_AWZS01000002.1
2955; Bradyrhizobium sp. ARR65 BraARR65DRAFT_scaffold_
9.10_C, whole genome shotgun sequence; 639168743;
NZ_AWZU01000010.1
2956; Paenibacillus sp. MAEPY2 contig7, whole genome
shotgun sequence; 639451286; NZ_AWUK01000007.1
2957; Verrucomicrobia bacterium LP2A
G346DRAFT_scf7180000000012_quiver.2_C, whole genome
shotgun sequence; 640169055; NZ_JAFS01000002.1
2958; Verrucomicrobia bacterium LP2A
G346DRAFT_scf7180000000012_quiver.2_C, whole genome
shotgun sequence; 640169055; NZ_JAFS01000002.1
2959; Robbsia andropogonis Ba3549 160, whole genome shotgun
sequence; 640451877; NZ_AYSW01000160.1
2960; Bacillus mannanilyticus JCM 10596, whole genome shotgun
sequence; 640600411; NZ_BAMO01000071.1
2961; Bacillus sp. H1a Contig1, whole genome shotgun sequence;
640724079; NZ_AYMH01000001.1
2962; Enterococcus faecalis ATCC 4200 supercont1.2, whole
genome shotgun sequence; 239948580; NZ_GG670372.1
2963; Enterococcus faecalis EnGen0363 strain RMC5 acAqY-
supercont1.4, whole genome shotgun sequence; 502232520;
NZ_KB944632.1
2964; Enterococcus faecalis LA3B-2 Scaffold22, whole genome
shotgun sequence; 522837181; NZ_KE352807.1
2965; Bifidobacterium breve NCFB 2258, complete genome;
749295448; NZ_CP006714.1
2966; Sphingomonas sanxanigenens NX02, complete genome;
749321911; NZ_CP006644.1
2967; Nocardia nova SH22a, complete genome; 753809381;
NZ_CP006850.1
2968; Kutzneria albida DSM 43870, complete genome; 754862786;
NZ_CP007155.1
2969; Paenibacillus polymyxa SQR-21, complete genome;
749205063; NZ_CP006872.1
2970; Burkholderia thailandensis E264 chromosome I, complete
sequence; 83718394; NC_007651.1
2971; Burkholderia thailandensis H0587 chromosome 1, complete
sequence; 759581710; NZ_CP004089.1
2972; Sphingobium barthaii strain KK22, whole genome shotgun
sequence; 646523831; NZ_BATN01000047.1
2973; Sphingobium barthaii strain KK22, whole genome shotgun
sequence; 646529442; NZ_BATN01000092.1
2974; Paenibacillus polymyxa 1-43 S143_contig00221, whole
genome shotgun sequence; 647225094; NZ_ASRZ01000173.1
2975; Paenibacillus sp. 1-49 5149_contig00281, whole genome
shotgun sequence; 647230448; NZ_ASRY01000102.1
2976; Paenibacillus graminis RSA19 S2_contig00597, whole
genome shotgun sequence; 647256651; NZ_ASSG01000304.1
2977; Paenibacillus sp. 1-18 S118_contig00103, whole genome
shotgun sequence; 647269417; NZ_ASSB01000031.1
2978; Paenibacillus polymyxa TD94 STD94_contig00759, whole
genome shotgun sequence; 647274605; NZ_ASSA01000134.1
2979; Bacillus flexus T6186-2 contig_106, whole genome shotgun
sequence; 647636934; NZ_JANV01000106.1
2980; Brevundimonas naejangsanensis strain B1 contig000018,
whole genome shotgun sequence; 647728918; NZ_JHOF01000018.1
2981; Burkholderia thailandensis E555 BTHE555_314, whole
genome shotgun sequence; 485035557; NZ_AECN01000315.1
2982; Burkholderia oklahomensis C6786 chromosome I, complete
sequence; 780352952; NZ_CP009555.1
2983; Bacillus endophyticus 2102 contig21, whole genome shotgun
sequence; 485049179; NZ_ALIM01000014.1
2984; Methylococcus capsulatus str. Texas = ATCC 19069 strain Texas
contig0129, whole genome shotgun sequence; 483090991;
NZ_AMCE01000064.1
2985; Sphingomonas-like bacterium B12, whole genome shotgun
sequence; 484115568; NZ_BACX01000797.1
2986; Nocardiopsis halotolerans DSM 44410 contig 372, whole
genome shotgun sequence; 484016556; NZ_ANAX01000372.1
2987; Nonomumea coxensis DSM 45129 A3G7DRAFT_scaffold_
4.5, whole genome shotgun sequence; 483454700; NZ_KB903974.1
2988; Streptomyces sp. CcalMP-8W B053DRAFT_scaffold_01,
whole genome shotgun sequence; 483961722; NZ_KB890915.1
2989; Spirosoma spitsbergense DSM 19989 B157DRAFT_scaffold_
76.77, whole genome shotgun sequence; 483994857; NZ_KB893599.1
2990; Butyrivibrio sp. XBB1001 G631DRAFT_scaffold00005.5_C,
whole genome shotgun sequence; 651376721; NZ_AUKA01000006.1
2991; Butyrivibrio sp. XPD2002 G587DRAFT_scaffold00011.11,
whole genome shotgun sequence; 651381584; NZ_KE384117.1
2992; Butyrivibrio sp. NC3005 G634DRAFT_scaffold00001.1,
whole genome shotgun sequence; 651394394; NZ_KE384206.1
2993; Butyrivibrio sp. MC2021 T359DRAFT_scaffold00010.10_C,
whole genome shotgun sequence; 651407979; NZ_JHXX01000011.1
2994; Paenarthrobacter nicotinovorans 231Sha2.1M6
I960DRAFT_scaffold00004.4_C, whole genome shotgun sequence;
651445346; NZ_AZVC01000006.1
2995; Bacillus sp. J37 BacJ37DRAFT_scaffold_0.1_C, whole
genome shotgun sequence; 651516582; NZ_JAEK01000001.1
2996; Bacillus sp. J37 BacJ37DRAFT_scaffold_0.1_C, whole
genome shotgun sequence; 651516582; NZ_JAEK01000001.1
2997; Bacillus sp. UNC437CL72CviS29 M014DRAFT_
scaffold00009.9_C, whole genome shotgun sequence; 651596980;
NZ_AXVB01000011.1
2998; Butyrivibrio sp. FC2001 G601DRAFT_scaffold00001.1,
whole genome shotgun sequence; 651921804; NZ_KE384132.1
2999; Bacillus bogoriensis ATCC BAA-922 T323DRAFT_
scaffold00008.8_C, whole genome shotgun sequence; 651937013;
NZ_JHYI01000013.1
3000; Bacillus bogoriensis ATCC BAA-922 T323DRAFT_
scaffold00008.8_C, whole genome shotgun sequence; 651937013;
NZ_JHYI01000013.1
3001; Bacillus kribbensis DSM 17871 H539DRAFT_scaffold00003.3,
whole genome shotgun sequence; 651983111; NZ_KE387239.1
3002; Fischerella sp. PCC 9431 Fis9431DRAFT_Scaffold1.2, whole
genome shotgun sequence; 652326780; NZ_KE650771.1
3003; Fischerella sp. PCC 9605 FIS9605DRAFT_scaffold2.2, whole
genome shotgun sequence; 652337551; NZ_KI912149.1
3004; Clostridium akagii DSM 12554 BR66DRAFT_scaffold00010.10_C,
whole genome shotgun sequence; 652488076; NZ_JMLK01000014.1
3005; Clostridium beijerinckii HUN142 T483DRAFT_scaffold00004.4,
whole genome shotgun sequence; 652494892; NZ_KK211337.1
3006; Glomeribacter sp. 1016415 H174DRAFT_scaffold00001.1, whole
genome shotgun sequence; 652527059; NZ_KE384226.1
3007; Glomeribacter sp. 1016415 H174DRAFT_scaffold00001.1, whole
genome shotgun sequence; 652527059; NZ_KE384226.1
3008; Mesorhizobium sp. URHA0056 H959DRAFT_scaffold00004.4_C,
whole genome shotgun sequence; 652670206; NZ_AUEL01000005.1
3009; Mesorhizobium loti R88b Meslo2DRAFT_Scaffold1.1, whole
genome shotgun sequence; 652688269; NZ_KI912159.1
3010; Mesorhizobium ciceri WSM4083 MESCI2DRAFT_scaffold_0.1,
whole genome shotgun sequence; 652698054; NZ_KI912610.1
3011; Mesorhizobium sp. URHC0008 N549DRAFT_scaffold00001.1_C,
whole genome shotgun sequence; 652699616; NZ_JIAP01000001.1
3012; Mesorhizobium sp. URHB0007 N550DRAFT_scaffold00001.1_C,
whole genome shotgun sequence; 652714310; NZ_JIA001000011.1
3013; Mesorhizobium erdmanii USDA 3471 A3AUDRAFT_
scaffold_7.8_C, whole genome shotgun sequence; 652719874;
NZ_AXAE01000013.1
3014; Mesorhizobium loti CJ3sym A3A9DRAFT_scaffold 25.26_C,
whole genome shotgun sequence; 652734503; NZ_AXAL01000027.1
3015; Cohnella thermotolerans DSM 17683 G485DRAFT_
scaffold00041.41_C, whole genome shotgun sequence; 652787974;
NZ_AUCP01000055.1
3016; Cohnella thermotolerans DSM 17683 G485DRAFT_
scaffold00041.41_C, whole genome shotgun sequence; 652787974;
NZ_AUCP01000055.1
3017; Cohnella thermotolerans DSM 17683 G485DRAFT_
scaffold00003.3, whole genome shotgun sequence; 652794305;
NZ_KE386956.1
3018; Lachnospiraceae bacterium NK4A144 G619DRAFT_
scaffold00002.2_C, whole genome shotgun sequence; 652826657;
NZ_AUJT01000002.1
3019; Mesorhizobium sp. WSM3626 Mesw3626DRAFT_scaffold_6.7_
C, whole genome shotgun sequence; 652879634; NZ_AZUY01000007.1
3020; Mesorhizobium sp. WSM1293 MesloDRAFT_scaffold_4.5,
whole genome shotgun sequence; 652910347; NZ_KI911320.1
3021; Mesorhizobium sp. WSM3224 YU3DRAFT_scaffold_3.4_C,
whole genome shotgun sequence; 652912253; NZ_ATYO01000004.1
3022; Butyrivibrio fibrisolvens MD2001 G635DRAFT_
scaffold00033.33_C, whole genome shotgun sequence; 652963937;
NZ_AUKD01000034.1
3023; Legionella pneumophila subsp. pneumophila strain ATCC 33155
contig032, whole genome shotgun sequence; 652971687;
NZ_JFIN01000032.1
3024; Legionella pneumophila subsp. pneumophila strain ATCC 33154
Scaffold2, whole genome shotgun sequence; 653016013; NZ_KK074241.1
3025; Legionella pneumophila subsp. pneumophila strain ATCC 33823
Scaffold7, whole genome shotgun sequence; 653016661; NZ_KK074199.1
3026; Bacillus sp. URHB0009 H980DRAFT_scaffold00016.16_C,
whole genome shotgun sequence; 653070042; NZ_AUER01000022.1
3027; Lachnospira multipara ATCC 19207 G600DRAFT_
scaffold00009.9_C, whole genome shotgun sequence;
653218978; NZ_AUJG01000009.1
3028; Lachnospira multipara MC2003 T520DRAFT_scaffold00007.7_C,
whole genome shotgun sequence; 653225243; NZ_JHWY01000011.1
3029; Rhodanobacter sp. OR87 RhoOR87DRAFT_scaffold_24.25S, whole
genome shotgun sequence; 653308965; NZ_AXBJ01000026.1
3030; Rhodanobacter sp. OR92 RhoOR92DRAFT_scaffold_6.7_C, whole
genome shotgun sequence; 653321547; NZ_ATYF01000013.1
3031; Rhodanobacter sp. OR444RHOOR444DRAFT
NODES len_27336_cov_289_843719.5_C, whole
genome shotgun sequence; 653325317; NZ_ATYD01000005.1
3032; Rhodanobacter sp. OR444 RHOOR444DRAFT_
NODE_39_len_52063_cov_320_872864.39, whole
genome shotgun sequence; 653330442; NZ_KE386531.1
3033; Bradyrhizobium sp. WSM1743 YU9DRAFT_scaffold_1.2_C, whole
genome shotgun sequence; 653526890; NZ_AXAZ01000002.1
3034; Bradyrhizobium sp. Ai1a-2 K288DRAFT_scaffold00086.86_C,
whole genome shotgun sequence; 653556699; NZ_AUEZ01000087.1
3035; Clostridium butyricum AGR2140 G607DRAFT_scaffold00008.8_C,
whole genome shotgun sequence; 653632769; NZ_AUJN01000009.1
3036; Mastigocoleus testarum BC008 Contig-2, whole genome
shotgun sequence; 959926096; NZ_LMTZ01000085.1
3037; [Eubacterium] cellulosolvens LD2006 T358DRAFT_
scaffold00002.2_C, whole genome shotgun sequence; 654392970;
NZ_JHXY01000005.1
3038; Desulfatiglans anilini DSM 4660 H567DRAFT_scaffold00005.5_
C, whole genome shotgun sequence; 654868823; NZ_AULM01000005.1
3039; Legionella pneumophila subsp. fraseri strain ATCC 35251
contig031, whole genome shotgun sequence; 654928151;
NZ_JFIG01000031.1
3040; Bacillus sp. FJAT-14578 Scaffold2, whole genome
shotgun sequence; 654948246; NZ_KI632505.1
3041; Bacillus sp. J13 PaeJ13DRAFT_scaffold_4.5_C, whole
sequence; 654954291; NZ_JAEO01000006.1
3042; Bacillus sp. 278922_107 H622DRAFT_scaffold00001.1,
whole genome shotgun sequence; 654964612; NZ_KI911354.1
3043; Streptomyces sp. GXT6 genomic scaffold Scaffold4, whole
genome shotgun sequence; 654975403; NZ_KI601366.1
3044; Ruminococcus flavefaciens ATCC 19208 L870DRAFT_
scaffold00001.1, whole genome shotgun sequence; 655069822;
NZ_KI912489.1
3045; Paenibacillus sp. UNCCL52 BR01DRAFT_scaffold00001.1,
whole genome shotgun sequence; 655095448; NZ_KK366023.1
3046; Paenibacillus sp. UNC451MF BP97DRAFT_scaffold00018.18_C,
whole genome shotgun sequence; 655103160; NZ_JMLS01000021.1
3047; Paenibacillus pinihumi DSM 23905 = JCM 16419 strain
DSM 23905 H583DRAFT_scaffold00005.5, whole genome shotgun
sequence; 655115689; NZ_KE383867.1
3048; Desulfobulbus japonicus DSM 18378 G493DRAFT_
scaffold00011.11_C, whole genome shotgun sequence; 655133038;
NZ_AUCV01000014.1
3049; Desulfobulbus mediterraneus DSM 13871
G494DRAFT_scaffold00028.28_C, whole genome shotgun sequence;
655138083; NZ_AUCW01000035.1
3050; Paenibacillus harenae DSM 16969 H581DRAFT_scaffold00002.2,
whole genome shotgun sequence; 655165706; NZ_KE383843.1
3051; Shimazuella kribbensis DSM 45090 A3GQDRAFT_scaffold_0.1_C,
whole genome shotgun sequence; 655370026; NZ_ATZF01000001.1
3052; Shimazuella kribbensis DSM 45090 A3GQDRAFT_scaffold_5.6_C,
whole genome shotgun sequence; 655371438; NZ_ATZF01000006.1
3053; Streptomyces flavidovirens DSM 40150 G412DRAFT_
scaffold00007.7_C, whole genome shotgun sequence; 655414006;
NZ_AUBE01000007.1
3054; Streptomyces flavidovirens DSM 40150 G412DRAFT_
scaffold00009.9, whole genome shotgun sequence; 655416831;
NZ_KE386846.1
3055; Terasakiella pusilla DSM 6293 Q397DRAFT_scaffold00039.39_C,
whole genome shotgun sequence; 655499373; NZ_JHYO01000039.1
3056; Pseudoxanthomonas suwonensis J43 Psesu2DRAFT_
scaffold_44.45_C, whole genome shotgun sequence; 655566937;
NZ_JAES01000046.1
3057; Pseudonocardia acaciae DSM 45401 N912DRAFT_
scaffold00002.2_C, whole genome shotgun sequence; 655569633;
NZ_JIAI01000002.1
3058; Azospirillum halopraeferens DSM 3675
G472DRAFT_scaffold00039.39_C, whole genome shotgun sequence;
655967838; NZ_AUCF01000044.1
3059; Clostridium scatologenes strain ATCC 25775, complete genome;
802929558; NZ_CP009933.1
3060; Paenibacillus harenae DSM 16969 H581DRAFT_
scaffold00004.4, whole genome shotgun sequence; 656245934;
NZ_KE383845.1
3061; Paenibacillus harenae DSM 16969 H581DRAFT_
scaffold00004.4, whole genome shotgun sequence; 656245934;
NZ_KE383845.1
3062; Paenibacillus alginolyticus DSM 5050 = NBRC 15375 strain
DSM 5050 G519DRAFT_scaffold00043.43_C, whole genome
shotgun sequence; 656249802; NZ_AUGY01000047.1
3063; Bacillus indicus strain DSM 16189 Contig01, whole genome
shotgun sequence; 737222016; NZ_JNVC02000001.1
3064; Acaryochloris sp. CCMEE 5410 contig00232, whole genome
shotgun sequence; 359367134; NZ_AFEJ01000154.1
3065; Bacillus sp. RP1137 contig_18, whole genome shotgun sequence;
657210762; NZ_AXZS01000018.1
3066; Streptomyces leeuwenhoekii strain C34(2013) c34_sequence_0501,
whole genome shotgun sequence; 657301257; NZ_AZSD01000480.1
3067; Brevundimonas bacteroides DSM 4726 Q333DRAFT_
scaffold00004.4_C, whole genome shotgun sequence; 657605746;
NZ_JNIX01000010.1
3068; Bacillus thuringiensis LM1212 scaffold_08, whole genome shotgun
sequence; 657629081; NZ_AYPV01000024.1
3069; Klebsiella pneumoniae 4541-2 4541_2_67, whole genome shotgun
sequence; 657698352; NZ_JDWO01000067.1
3070; Lachnoclosritidium phytofermentans KNHs212
B010DRAFT_scf7180000000004_quiver.1_C, whole genome shotgun
sequence; 657706549; NZ_JNLM01000001.1
3071; Paenibacillus polymyxa strain WLY78 S6_contig00095, whole
genome shotgun sequence; 657719467; NZ_ALJV01000094.1
3072; Bacillus indicus strain DSM 16189 Contig01, whole genome
shotgun sequence; 737222016; NZ_JNVC02000001.1
3073; [Scytonema hofmanni] UTEX 2349 Tol9009DRAFT_TPD.8, whole
genome shotgun sequence; 657935980; NZ_KK073768.1
3074; Caulobacter sp. UNC358MFTsu5.1 BR39DRAFT_
scaffold00002.2_C, whole genome shotgun sequence; 659864921;
NZ_JONW01000006.1
3075; Sphingomonas sp. YL-JM2C contig056, whole genome
shotgun sequence; 661300723; NZ_ASTM01000056.1
3076; Streptomyces monomycini strain NRRL B-24309
P063_Doro1_scaffold135, whole genome shotgun sequence;
662059070; NZ_KL571162.1
3077; Streptomyces flavotricini strain NRRL B-5419 contig237.1,
whole genome shotgun sequence; 662063073; NZ_JNXV01000303.1
3078; Streptomyces peruviensis strain NRRL ISP-5592 P181_Doro1_
scaffold152, whole genome shotgun sequence; 662097244;
NZ_KL575165.1
3079; Sphingomonas sp. DC-6 scaffold87, whole genome shotgun
sequence; 662140302; NZ_JMUB01000087.1
3080; Streptomyces sp. NRRL S-455 contig1.1, whole genome
shotgun sequence; 663192162; NZ_JOCT01000001.1
3081; Streptomyces griseoluteus strain NRRL ISP-5360 contig43.1,
whole genome shotgun sequence; 663180071; NZ_JOBE01000043.1
3082; Streptomyces sp. NRRL S-350 contig12.1, whole genome
shotgun sequence; 663199697; NZ_JOH001000012.1
3083; Streptomyces katrae strain NRRL B-16271 contig37.1, whole
genome shotgun sequence; 663300941; NZ_JNZY01000037.1
3084; Streptomyces sp. NRRL B-3229 contig5.1, whole genome
shotgun sequence; 663316931; NZ_JOGP01000005.1
3085; Streptomyces flavochromogenes strain NRRL B-2684
contig8.1, whole genome shotgun sequence; 663317502;
NZ_JNZ001000008.1
3086; Streptomyces roseoverticillatus strain NRRL B-3500
contig22.1, whole genome shotgun sequence; 663372343;
NZ_JOFL01000022.1
3087; Streptomyces roseoverticillatus strain NRRL B-3500
contig31.1, whole genome shotgun sequence; 663372947;
NZ_JOFL01000031.1
3088; Streptomyces roseoverticillatus strain NRRL B-3500
contig43.1, whole genome shotgun sequence; 663373497;
NZ_JOFL01000043.1
3089; Streptomyces rimosus subsp. rimosus strain NRRL
WC-3924 contig19.1, whole genome shotgun sequence;
663376433; NZ_JOBW01000019.1
3090; Streptomyces rimosus subsp. rimosus strain NRRL
WC-3924 contig82.1, whole genome shotgun sequence;
663379797; NZ_JOBW01000082.1
3091; Streptomyces sp. NRRL B-12105 contig1.1, whole genome
shotgun sequence; 663380895; NZ_JNZW01000001.1
3092; Herbidospora cretacea strain NRRL B-16917 contig7.1,
whole genome shotgun sequence; 663670981; NZ_JODQ01000007.1
3093; Lechevalieria aerocolonigenes strain NRRL B-3298 contig27.1,
whole genome shotgun sequence; 663693444; NZ_JOFI01000027.1
3094; Microbispora rosea subsp. nonnitritogenes strain NRRL
B-2631 contig12.1, whole genome shotgun sequence; 663732121;
NZ_JNZQ01000012.1
3095; Sphingobium sp. DC-2 ODE_45, whole genome shotgun
6sequence; 63818579; NZ_JNAC01000042.1
3096; Streptomyces aureocirculatus strain NRRL ISP-5386 contig49.1,
whole genome shotgun sequence; 664026629; NZ_JOAP01000049.1
3097; Streptomyces rimosus subsp. rimosus strain NRRL
B-2660 contig14.1, whole genome shotgun sequence; 664052786;
NZ_JOES01000014.1
3098; Streptomyces achromogenes subsp. achromogenes strain
NRRL B-2120 contig2.1, whole genome shotgun sequence;
664063830; NZ_JODT01000002.1
3099; Streptomyces rimosus subsp. rimosus strain NRRL B-2660
contig124.1, whole genome shotgun sequence; 664066234;
NZ_JOES01000124.1
3100; Streptomyces rimosus subsp. rimosus strain NRRL WC-3927
contig5.1, whole genome shotgun sequence; 664091759;
NZ_JOBO01000005.1
3101; Streptomyces rimosus subsp. rimosus strain NRRL WC-3869
P248contig50.1, whole genome shotgun sequence; 925315417;
LGCQ01000244.1
3102; Streptomyces rimosus subsp. rimosus strain NRRL
WC-3929 contig5.1, whole genome shotgun sequence;
664104387; NZ_J0JJ01000005.1
3103; Streptomyces rimosus subsp. rimosus strain NRRL
WC-3929 contig46.1, whole genome shotgun sequence;
664115745; NZ__JOJJ01000046.1
3104; Streptomyces rimosus subsp. rimosus strain NRRL
WC-3904 contig10.1, whole genome shotgun sequence;
664126885; NZ_JOCQ01000010.1
3105; Streptomyces rimosus subsp. rimosus strain NRRL
WC-3904 contig106.1, whole genome shotgun sequence;
664141810; NZ_JOCQ01000106.1
3106; Streptomyces sp. NRRL F-2890 contig2.1, whole genome
shotgun sequence; 664194528; NZ_JOIG01000002.1
3107; Streptomyces griseus subsp. griseus strain NRRL F-5618
contig4.1, whole genome shotgun sequence; 664233412;
NZ_JOGN01000004.1
3108; Streptomyces lavenduligriseus strain NRRL ISP-5487 contig2.1,
whole genome shotgun sequence; 664244706; NZ_JOBD01000002.1
3109; Streptomyces sp. NRRL S-920 contig3.1, whole genome
shotgun sequence; 664245663; NZ_JODF01000003.1
3110; Streptomyces hygroscopicus subsp. hygroscopicus strain
NRRL B-1477 contig8.1, whole genome shotgun sequence;
664299296; NZ_JOIK01000008.1
3111; Streptomyces sp. NRRL F-4474 contig32.1, whole genome
shotgun sequence; 664323078; NZ_JOIB01000032.1
3112; Streptomyces sp. NRRL S-475 contig32.1, whole genome
shotgun sequence; 664325162; NZ_JOJB01000032.1
3113; Streptomyces sp. NRRL F-5053 contig1.1, whole genome
shotgun sequence; 664356765; NZ_JOHT01000001.1
3114; Streptomyces sp. NRRL S-1868 contig54.1, whole genome
shotgun sequence; 664360925; NZ_JOGD01000054.1
3115; Streptomyces sp. NRRL S-646 contig23.1, whole genome
shotgun sequence; 664421883; NZ_JODC01000023.1
3116; Streptomyces sp. NRRL S-455 contig1.1, whole genome
shotgun sequence; 663192162; NZ_JOCT01000001.1
3117; Streptomyces sp. NRRL S-481 P269_Doro1_scaffold20,
whole genome shotgun sequence; 664428976; NZ_KL585179.1
3118; Streptomyces sp. NRRL F-5140 contig927.1, whole
genome shotgun sequence; 664434000; NZ_JOIA01001078.1
3119; Streptomyces sp. NRRL WC-3773 contig2.1, whole
genome shotgun sequence; 664478668; NZ_JOJI01000002.1
3120; Streptomyces sp. NRRL WC-3773 contig5.1, whole
genome shotgun sequence; 664479796; NZ_JOJI01000005.1
3121; Streptomyces sp. NRRL WC-3773 contig11.1, whole
genome shotgun sequence; 664481891; NZ_JOJI01000011.1
3122; Streptomyces sp. NRRL WC-3773 contig11.1, whole
genome shotgun sequence; 664481891; NZ_JOJI01000011.1
3123; Streptomyces puniceus strain NRRL ISP-5083 contig3.1,
whole genome shotgun sequence; 663149970; NZ_JOBQ01000003.1
3124; Streptomyces ochraceiscleroticus strain NRRL ISP-5594
contig9.1, whole genome shotgun sequence; 664540649;
NZ_JOAX01000009.1
3125; Streptomyces durhamensis strain NRRL B-3309 contig3.1,
whole genome shotgun sequence; 665586974; NZ_JNXR01000003.1
3126; Streptomyces durhamensis strain NRRL B-3309 contig23.1,
whole genome shotgun sequence; 665604093; NZ_JNXR01000023.1
3127; Streptomyces rimosus subsp. rimosus strain NRRL WC-3869
P248contig20.1, whole genome shotgun sequence; 925322461;
LGCQ01000113.1
3128; Streptomyces niveus NCIMB 11891 chromosome, whole
genome shotgun sequence; 566146291; NZ_CM002280.1
3129; Paenibacillus polymyxa strain CICC 10580 contig_11,
whole genome shotgun sequence; 670516032; NZ_JNCB01000011.1
3130; Streptomyces megasporus strain NRRL B-16372 contig19.1,
whole genome shotgun sequence; 671525382; NZ_JODL01000019.1
3131; Dyadobacter crusticola DSM 16708 Q369DRAFT_
scaffold00002.2, whole genome shotgun sequence; 671546962;
NZ_KL370786.1
3132; Bacillus sp. MB2021 T349DRAFT_scaffold00010.10_C,
whole genome shotgun sequence; 671553628; NZ_JNJJ01000011.1
3133; Lachnospira multipara LB2003 T537DRAFT_
scaffold00010.10_C, whole genome shotgun sequence; 671578517;
NZ_JNKW01000011.1
3134; Clostridium drakei strain SL1 contig 20, whole genome
shotgun sequence; 692121046; NZ_JIBU02000020.1
3135; Candidatus Paracaedibacter symbiosus strain PRA9 Scaffold 1,
whole genome shotgun sequence; 692233141; NZ_JQAK01000001.1
3136; Stenotrophomonas maltophilia strain 53 contig_2, whole
genome shotgun sequence; 692316574; NZ_JRJA01000002.1
3137; Rhodococcus fascians LMG 3625 contig38, whole genome
shotgun sequence; 694033726; NZ_JMEM01000016.1
3138; Rhodococcus fascians 04-516 contig54, whole genome
shotgun sequence; 694058371; NZ_JMFD01000020.1
3139; Klebsiella michiganensis strain R8A contig_44, whole
genome shotgun sequence; 695806661; NZ_JNCH01000044.1
3140; Streptomyces globisporus C-1027 Scaffold24_1, whole
genome shotgun sequence; 410651191; NZ_AJUO01000171.1
3141; Streptomyces sp. NRRL B-1381 contig33.1, whole genome
shotgun sequence; 663334964; NZ_JOHG01000033.1
3142; Streptomyces sp. SolWspMP-sol2th B083DRAFT_
scaffold_17.18_C, whole genome shotgun sequence; 654969845;
NZ_ARPF01000020.1
3143; Streptomyces alboviridis strain NRRL B-1579 contig18.1,
whole genome shotgun sequence; 695845602; NZ_JNWU01000018.1
3144; Streptomyces sp. NRRL F-5681 contig10.1, whole genome
shotgun sequence; 663292631; NZ_JOHA01000010.1
3145; Streptomyces globisporus subsp. globisporus strain
NRRL B-2709 contig24.1, whole genome shotgun sequence;
664051798; NZ_JNZK01000024.1
3146; Streptomyces griseus subsp. griseus strain NRRL
F-5144 contig19.1, whole genome shotgun sequence;
664184565; NZ_JOGA01000019.1
3147; Streptomyces floridar strain NRRL 2423 contig7.1, whole
genome shotgun sequence; 663343774; NZ_JOAC01000007.1
3148; Streptomyces roseosporus NRRL 11379 supercont4.1, whole
genome shotgun sequence; 588273405; NZ_ABYX02000001.1
3149; Streptomyces cyaneofuscatus strain NRRL B-2570 contig9.1,
whole genome shotgun sequence; 664021017; NZ_JOEM01000009.1
3150; Streptomyces sp. NRRL S-623 contig14.1, whole genome
shotgun sequence; 665522165; NZ_JOJC01000016.1
3151; Streptomyces sp. JS01 contig2, whole genome shotgun
sequence; 695871554; NZ_JPWW01000002.1
3152; Streptomyces albus subsp. albus strain NRRL B-2445
contig28.1, whole genome shotgun sequence; 664095100;
NZ_JOED01000028.1
3153; Streptomyces baamensis strain NRRL B-2842 P144_
Doro1_scaffold26, whole genome shotgun sequence;
662135579; NZ_KL573564.1
3154; Streptomyces griseus subsp. griseus strain NRRL F-2227
contig41.1, whole genome shotgun sequence;
664325626; NZ_JOIT01000041.1
3155; Streptomyces sp. W007 contig00293, whole genome
shotgun sequence; 365867746; NZ_AGSW01000272.1
3156; Streptomyces mediolani strain NRRL WC-3934 contig31.1,
whole genome shotgun sequence; 664285409;
NZ_JOJK01000031.1
3157; Streptomyces sp. NRRL WC-3773 contig36.1, whole
genome shotgun sequence; 664487325; NZ_JOJI01000036.1
3158; Mesorhizobium japonicum R7A MesloDRAFT_Scaffold1.1,
whole genome shotgun sequence; 696358903; NZ_KI632510.1
3159; Stenotrophomonas maltophilia RA8, whole genome
shotgun sequence; 493412056; NZ_CALM01000701.1
3160; Streptomyces rimosus subsp. rimosus strain NRRL
B-2660 contig59.1, whole genome shotgun sequence;
664061406; NZ_JOES01000059.1
3161; Streptomyces rimosus subsp. rimosus strain NRRL
B-16073 contig7.1, whole genome shotgun sequence;
696493030; NZ_JNWX01000007.1
3162; Streptomyces rimosus subsp. rimosus strain NRRL
B-16073 contig48.1, whole genome shotgun sequence;
696497741; NZ_JNWX01000048.1
3163; Sphingopyxis sp. MWB1 contig00002, whole genome
shotgun sequence; 696542396; NZ_JQFJ01000002.1
3164; Blautia producta strain ER3 contig_8, whole genome
shotgun sequence; 696661199; NZ_JPJF01000008.1
3165; Streptomyces griseus subsp. griseus strain NRRL B-2307
contig15.1, whole genome shotgun sequence; 702684649;
NZ_JNZI01000015.1
3166; Kitasatospora setae KM-6054 DNA, complete genome;
NC_016109.1
3167; Streptomyces lydicus strain NRRL ISP-5461 contig41.1,
whole genomes hotgun sequence; 702808005; NZ_JNZA01000041.1
3168; Streptomyces iakyrus strain NRRL ISP-5482 contig6.1,
whole genome shotgun sequence; 702914619; NZ_JNXI01000006.1
3169; Kibdelosporangium aridum subsp. largum strain NRRL
B-24462 contig4.56, whole genome shotgun sequence; 703210604;
NZ_JNYM01000124.1
3170; Kibdelosporangium aridum subsp. largum strain NRRL
B-24462 contig91.4, whole genome shotgun sequence;
703243970; NZ_JNYM01001429.1
3171; Xanthomonas campestris pv. viticola strain LMG 965,
whole genome shotgun sequence; 704493846;
NZ_CBZT010000006.1
3172; Streptomyces galbus strain KCCM 41354 contig00021,
whole genome shotgun sequence; 716912366; NZ_JRHJ01000016.1
3173; Bacillus aryabhattai strain GZ03 contig1_scaffold1, whole
genome shotgun sequence; 723602665; NZ_JPIE01000001.1
3174; Bacillus mycoides FSL H7-687 Contig052, whole genome
shotgun sequence; 727271768; NZ_ASPY01000052.1
3175; Bacillus mycoides strain Flugge 10206 DJ94.contig-100_16,
whole genome shotgun sequence; 727343482; NZ_JMQD01000030.1
3176; Streptomyces anulatus strain NRRL B-2873 contig21.1,
whole genome shotgun sequence; 664049400; NZ_JOEZ01000021.1
3177; Sphingomonas sp. 37zxx contig3_scaffold2, whole
genome shotgun sequence; 728813405; NZ_JROH01000003.1
3178; Sphingomonas sp. 35-24ZXX contig00_scaffold4, whole
genome shotgun sequence; 728827031; NZ_JROG01000008.1
3179; Sphingomonas sp. Ant H11 contig_149, whole genome
shotgun sequence; 730274767; NZ_JSBN01000149.1
3180; Sphingomonas sp. ERG5 Contig74, whole genome
shotgun sequence; 734983081; NZ_JSXI01000073.1
3181; Sphingomonas sp. ERG5 Contig80, whole genome
shotgun sequence; 734983422; NZ_JSXI01000079.1
3182; Bacillus sp. 72 T409DRAFT_scf7180000000077_quiver.
15_C, whole genome shotgun sequence; 736160933;
NZ_JQMI01000015.1
3183; Bacillus simplex BA2H3 scaffold2, whole genome
shotgun sequence; 736214556; NZ_KN360955.1
3184; Dehalobacter sp. UNSWDHB Contig_139, whole genome
shotgun sequence; 544905305; NZ_AUUR01000139.1
3185; Bacillus manliponensis strain JCM 15802 contig4, whole
genome shotgun sequence; 736629899; NZ_JOTN01000004.1
3186; Hyphomonas chukchiensis strain BH-BN04-4 contig6,
whole genome shotgun sequence; 736739493;
NZ_AWFG01000063.1
3187; Bacillus vietnamensis strain HD-02, whole genome
shotgun sequence; 736762362; NZ_CCDN010000009.1
3188; Hyphomonas sp. CY54-11-8 contig4, whole genome
shotgun sequence; 736764136; NZ_AWFD01000033.1
3189; Erythrobacter longus strain DSM 6997 contig9, whole
genome shotgun sequence; 736965849; NZ_JMEW01000009.1
3190; Caulobacter henficii strain CF287 EW90DRAFT
scaffold00023.23_C, whole genome shotgun sequence;
737089868; NZ_JQJN01000025.1
3191; Caulobacter henficii strain YR570 EX13DRAFT_
scaffold00022.22_C, whole genome shotgun sequence;
737103862; NZ_JQJP01000023.1
3192; Calothrix sp. 336/3, complete genome; 821032128;
NZ_CP011382.1
3193; Desulfobacter vibfiofonnis DSM 8776 Q366DRAFT_
scaffold00036.35_C, whole genome shotgun sequence;
737257311; NZ_JQKJ01000036.1
3194; Actinokineospora spheciospongiae strain EG49
contig1268_1, whole genome shotgun sequence; 737301464;
NZ_AYXG01000139.1
3195; Brevundimonas sp. EAKA contig5, whole genome
shotgun sequence; 737322991; NZ_JMQR01000005.1
3196; Brevundimonas sp. EAKA contig5, whole genome
shotgun sequence; 737322991; NZ_JMQR01000005.1
3197; Brevundimonas sp. EAKA contig12, whole genome
shotgun sequence; 737323704; NZ_JMQR01000012.1
3198; Bacillus firmus DS1 scaffold33, whole genome shotgun
sequence; 737350949; NZ_APVL01000034.1
3199; Bacillus hemicellulosilyticus JCM 9152, whole genome
shotgun sequence; 737360192; NZ_BAUU01000008.1
3200; Edaphobacter aggregans DSM 19364 Q363DRAFT_
scaffold00032.32_C, whole genome shotgun sequence;
737370143; NZ_JQKI01000040.1
3201; Bacillus sp. UNC322MFChir4.1 BR72DRAFT_
scaffold00004.4, whole genome shotgun sequence; 737456981;
NZ_KN050811.1
3202; Haloglycomyces albus DSM 45210 HalalDRAFT_
chromosome1.1_C, whole genome shotgun sequence; 644043488;
NZ_AZUQ01000001.1
3203; Hyphomonas oceanitis SCH89 contig20, whole genome
shotgun sequence; 737567115; NZ_ARYL01000020.1
3204; Hyphomonas oceanitis SCH89 contig59, whole genome
shotgun sequence; 737569369; NZ_ARYL01000059.1
3205; Halobacillus sp. BBL2006 cont444, whole genome
shotgun sequence; 737576092; NZ_JRNX01000441.1
3206; Hyphomonas atlantica strain 22II1-22F38 contig10, whole
genome shotgun sequence; 737577234; NZ_AWFH01000002.1
3207; Hyphomonas atlantica strain 22II1-22F38 contig28, whole
genome shotgun sequence; 737580759; NZ_AWFH01000021.1
3208; Hyphomonas jannaschiana VP2 contig2, whole genome
shotgun sequence; 737608363; NZ_ARYJ01000002.1
3209; Bacillus akibai JCM 9157, whole genome shotgun
sequence; 737696658; NZ_BAUV01000025.1
3210; Frankia sp. CcI6 CcI6DRAFT_scaffold_16.17, whole
genome shotgun sequence; 564016690; NZ_AYTZ01000017.1
3211; Frankia sp. Thr ThrDRAFT_scaffold_48.49, whole
genome shotgun sequence; 602261491; JENI01000049.1
3212; Frankia sp. Thr ThrDRAFT_scaffold 28.29, whole
genome shotgun sequence; 602262270; JENI01000029.1
3213; Frankia sp. CcI6 CcI6DRAFT_scaffold_16.17, whole
genome shotgun sequence; 564016690; NZ_AYTZ01000017.1
3214; [Leptolyngbya] sp. JSC-1
Osccy1DRAFT_CYJSC1_DRAF_scaffold00069.1, whole
genome shotgun sequence; 738050739; NZ_KL662191.1
3215; Lysobacter darjeonensis GH1-9 contig23, whole genome
shotgun sequence; 738180952; NZ_AVPU01000014.1
3216; Myxosarcina sp. GI1 contig_5, whole genome shotgun
sequence; 738529722; NZ_JRFE01000006.1
3217; Novosphingobium resinovorum strain KF1 contig000002,
whole genome shotgun sequence; 738613868; NZ_JFYZ01000002.1
3218; Novosphingobium resinovorum strain KF1 contig000008,
whole genome shotgun sequence; 738615271; NZ_JFYZ01000008.1
3219; Novosphingobium resinovorum strain KF1 contig000015,
whole genome shotgun sequence; 738617000; NZ_JFYZ01000015.1
3220; Paenibacillus sp. FSL H7-689 Contig015, whole genome
shotgun sequence; 738716739; NZ_ASPU01000015.1
3221; Paenibacillus wynnii strain DSM 18334 unitig_2, whole
genome shotgun sequence; 738760618; NZ_JQCR01000002.1
3222; Pandoraea sp. SD6-2 scaffold29, whole genome
shotgun sequence; 505733815; NZ_KB944444.1
3223; Paenibacillus sp. FSL R7-269 Contig022, whole genome
shotgun sequence; 738803633; NZ_ASPS01000022.1
3224; Paenibacillus taiwanensis DSM 18679 H509DRAFT_
scaffold00010.10_C, whole genome shotgun sequence; 655095554;
NZ_AULE01000001.1
3225; Paenibacillus sp. FSL R7-277 Contig088, whole genome
shotgun sequence; 738841140; NZ_ASPX01000088.1
3226; Prevotella oryzae DSM 17970 XylorDRAFT_XOA.1, whole
genome shotgun sequence; 738999090; NZ_KK073873.1
3227; Promicromonospora kroppenstedtii DSM 19349 ProkrDRAFT_
PKA.71, whole genome shotgun sequence; 739097522; NZ_KI911740.1
3228; Pseudonocardia acaciae DSM 45401 N912DRAFT_
scaffold00002.2_C, whole genome shotgun sequence; 655569633;
NZ_JIAI01000002.1
3229; Rhodanobacter sp. 115 contig437, whole genome
shotgun sequence; 389759651; NZ_AJXS01000437.1
3230; Rhodococcus fascians A21d2 contig10, whole genome
shotgun sequence; 739287390; NZ_JMFA01000010.1
3231; Ruminococcus albus 8 contig00035, whole genome
shotgun sequence; 325680876; NZ_ADKM02000123.1
3232; Rubellimicrobium mesophilum DSM 19309 scaffold23,
whole genome shotgun sequence; 739419616; NZ_KK088564.1
3233; Rothia aeria F0184 Scaffold136, whole genome shotgun
sequence; 553804563; NZ_K1518028.1
3234; Amycolatopsis orientalis DSM 40040 = KCTC 9412 contig_32,
whole genome shotgun sequence; 499136900; NZ_ASJB01000015.1
3235; Amycolatopsis sp. MJM2582 contig00007, whole
genome shotgun sequence; 739487309; NZ_JPLW01000007.1
3236; Sphingobium chlorophenolicum strain NBRC 16172
contig000025, whole genome shotgun sequence; 739594477;
NZ_JFHR01000025.1
3237; Sphingobium chlorophenolicum strain NBRC 16172
contig000062, whole genome shotgun sequence; 739598481; NZ_
JFHR01000062.1
3238; Sphingobium herbicidovorans NBRC 16415 contig000028,
whole genome shotgun sequence; 739610197; NZ_JFZA02000028.1
3239; Sphingobium sp. ba1 seq0028, whole genome shotgun
sequence; 739622900; NZ_JPPQ01000069.1
3240; Sphingobium sp. ba1 seq0028, whole genome shotgun
sequence; 739622900; NZ_JPPQ01000069.1
3241; Sphingomonas paucimobilis strain EPA505 contig000016,
whole genome shotgun sequence; 739629085; NZ_JFYY01000016.1
3242; Sphingobium japonicum BiD32, whole genome
shotgun sequence; 494022722; NZ_CAVK010000217.1
3243; Sphingobium yanoikuyae strain B1 scaffold1, whole
genome shotgun sequence; 739650776; NZ_KL662193.1
3244; Sphingobium yanoikuyae strain B1 scaffold28, whole
genome shotgun sequence; 739656825; NZ_KL662220.1
3245; Sphingopyxis sp. LC81 contig24, whole genome shotgun
sequence; 739659070; NZ_JNFD01000017.1
3246; Sphingobium yanoikuyae strain B1 contig000019, whole
genome shotgun sequence; 739665456; NZ_JGVR01000019.1
3247; Sphingomonas wittichii strain YR128 EX04DRAFT_
scaffold00050.50_C, whole genome shotgun sequence; 739674258;
NZ_JQMC01000050.1
3248; Sphingopyxis fiibergensis strain Kp5.2, complete
genome; 749188513; NZ_CP009122.1
3249; Sphingopyxis sp. LC363 contig30, whole genome
shotgun sequence; 739701660; NZ_JNFC01000024.1
3250; Sphingopyxis sp. LC363 contig36, whole genome
shotgun sequence; 739702045; NZ_JNFC01000030.1
3251; Sphingopyxis sp. LC363 contig5, whole genome
shotgun sequence; 739702995; NZ_JNFC01000045.1
3252; Spirillospora albida strain NRRL B-3350 contig1.1, whole
genome shotgun sequence; 663122276; NZ_JOFJ01000001.1
3253; Sphingomonas sp. UNC305MFCo15.2 BR78DRAFT_
scaffold00001.1_C, whole genome shotgun sequence; 659889283;
NZ_JOOE01000001.1
3254; Streptococcus salivarius strain NU10 contig_11, whole
genome shotgun sequence; 739748927; NZ_JJMT01000011.1
3255; Streptomyces katrae strain NRRL B-16271 contig33.1,
whole genome shotgun sequence; 663300513; NZ_
JNZY01000033.1
3256; Streptomyces avermitilis MA-4680 = NBRC 14893,
complete genome; 162960844, NC_003155.4
3257; Streptomyces avermitilis MA-4680 = NBRC 14893,
complete genome; 162960844; NC_003155.4
3258; Streptomyces aurantiacus JA 4570 Seq17, whole genome
shotgun sequence; 514916021; NZ_AOPZ01000017.1
3259; Streptomyces griseus subsp. griseus strain NRRL WC-3645
contig39.1, whole genome shotgun sequence; 739830131;
NZ_JOJE01000039.1
3260; Streptomyces griseus subsp. griseus strain NRRL
WC-3645 contig40.1, whole genome shotgun
sequence; 739830264; NZ_JOJE01000040.1
3261; Streptomyces aureocirculatus strain NRRL ISP-5386 contig11.1,
whole genome shotgun sequence; 664013282; NZ_JOAP01000011.1
3262; Streptomyces scabiei strain NCPPB 4086 scf_65433_365.1,
whole genome shotgun sequence; 739854483; NZ_KL997447.1
3263; Streptomyces sp. ATexAB-D23 B082DRAFT_scaffold_0.1,
whole genome shotgun sequence; 483975550; NZ_KB892001.1
3264; Streptomyces lavendulae strain Fujisawa #8006 contig417.1,
whole genome shotgun sequence; 662043624; NZ_JNXL01000469.1
3265; Streptomyces sp. DpondAA-B6 K379DRAFT_
scaffold00015.15_C, wholegenome shotgun sequence;
654993549; NZ_AZVE01000016.1
3266; Streptomyces sclerotialus strain NRRL B-2317 contig7.1,
whole genome shotgun sequence; 664034500; NZ_JODX01000007.1
3267; Streptomyces olindensis strain DAUFPE 5622 103, whole
genome shotgun sequence; 739918964; NZ_JJOH01000097.1
3268; Streptomyces pristinaespiralis ATCC 25486 chromosome,
whole genome shotgun sequence; 297189896; NZ_CM000950.1
3269; Streptomyces sp. CNH099 B121DRAFT_scaffold_
16.17_C, whole genome shotgun sequence; 654239557;
NZ_AZWL01000018.1
3270; Streptomyces sp. MspMP-M5 B073DRAFT_scaffold_
27.28, whole genome shotgun sequence; 483974021;
NZ_KB891893.1
3271; Streptomyces sp. NRRL S-1813 contig13.1, whole
genome shotgun sequence; 664466568; NZ_JOHB01000013.1
3272; Streptomyces sp. NRRL S-87 contig69.1, whole genome
shotgun sequence; 663169513; NZ_JO
3273; Streptomyces sp. PRh5 contig001, whole genome
shotgun sequence; 740097110; NZ_JABQ01000001.1
3274; Thioclava dalianensis strain DLFJ1-1 contig2, whole
genome shotgun sequence; 740220529; NZ_JHEH01000002.1
3275; Tolypothrix bouteillei VB521301 scaffold_1, whole
genome shotgun sequence; 910242069; NZ_JHEG02000048.1
3276; Thioclava indica strain DT23-4 contig29, whole genome
shotgun sequence; 740292158; NZ_AUNB01000028.1
3277; Streptomyces albulus strain NK660, complete genome;
754221033; NZ_CP007574.1
3278; Paenibacillus sp. FSL H7-0357, complete genome;
749299172; NZ_CP009241.1
3279; Paenibacillus stellifer strain DSM 14472, complete
genome; 753871514; NZ_CP009286.1
3280; Burkholderia pseudomallei 1258a Contig0089, whole
genome shotgun sequence; 418540998; NZ_AHJB01000089.1
3281; Burkholderia pseudomallei ABCPW 91 scaffold1, whole
genome shotgun sequence; 740941050; NZ_KN323016.1
3282; Burkholderia pseudomallei strain MSHR4018 scaffold2,
whole genome shotgun sequence; 740942724; NZ_KN323080.1
3283; Burkholderia pseudomallei MSHR1357 scaffold1, whole
genome shotgun sequence; 740944663; NZ_KN323054.1
3284; Burkholderia pseudomallei ABCPW 30 scaffold1, whole
genome shotgun sequence; 740947478; NZ_KN323024.1
3285; Burkholderia pseudomallei MSHR465J scaffold1, whole
genome shotgun sequence; 740992312; NZ_KN322994.1
3286; Burkholderia pseudomallei T5V32 Y025.contig-100_19,
whole genome shotgun sequence; 740951623; NZ_JQHT01000093.1
3287; Burkholderia pseudomallei MSHR2990 scaffold2, whole
genome shotgun sequence; 740957131; NZ_KN323051.1
3288; Burkholderia sp. ABCPW 111 X946.contig-100_0, whole
genome shotgun sequence; 740958729; NZ_JPWT01000001.1
3289; Burkholderia pseudomallei MSHR1000 scaffold1, whole
genome shotgun sequence; 740963677; NZ_KN323065.1
3290; Burkholderia pseudomallei strain BDM scaffold1, whole
genome shotgun sequence; 740964046; NZ_KN150935.1
3291; Burkholderia pseudomallei strain BEG scaffold1, whole
genome shotgun sequence; 740978899; NZ_KN150957.1
3292; Burkholderia pseudomallei strain BDZ scaffold40, whole
genome shotgun sequence; 740989169; NZ_KN150904.1
3293; Burkholderia pseudomallei MSHR4377 scaffold1, whole
genome shotgun sequence; 740998359; NZ_KN322996.1
3294; Burkholderia pseudomallei strain BGH scaffold1, whole
genome shotgun sequence; 741001323; NZ_KN150943.1
3295; Burkholderia pseudomallei MSHR7343 X962.contig-100_
14, whole genome shotgun sequence; 741003124;
NZ_JQDM01000047.1
3296; Burkholderia pseudomallei strain PFGE_B T6 scaffold1,
whole genome shotgun sequence; 741007242; NZ_KN150983.1
3297; Burkholderia pseudomallei MSHR3965 chromosome
1 sequence; 752520733; NZ_CP009153.1
3298; Burkholderia pseudomallei MSHR5492.X992.contig-100_
0, whole genome shotgun sequence; 741015160;
NZ_JQD001000001.1
3299; Burkholderia oklahomensis strain EO147 chromosome
1, complete sequence; 752612400; NZ_CP008726.1
3300; Burkholderia oklahomensis strain EO147 chromosome
1, complete sequence; 752612400; NZ_CP008726.1
3301; Cupriavidus sp. IDO NODE_7, whole genome
shotgun sequence; 742878908; NZ_JWMA01000006.1
3302; Cupriavidus sp. IDO NODE_7, whole genome
shotgun sequence; 742878908; NZ_JWMA01000006.1
3303; Escherichia coli strain EC2_3 Contig93, whole genome
shotgun sequence; 742921760; NZ_JWKL01000093.1
3304; Brevundimonas nasdae strain TPW30 Contig_11, whole
genome shotgun sequence; 746187486; NZ_JWSY01000011.1
3305; Brevundimonas nasdae strain TPW30 Contig_13, whole
genome shotgun sequence; 746187665; NZ_JWSY01000013.1
3306; Paenibacillus polymyxa strain DSM 365 Contig001, whole
genome shotgun sequence; 746220937; NZ_JMIQ01000001.1
3307; Paenibacillus polymyxa strain CF05 genome; 746228615;
NZ_CP009909.1
3308; Novosphingobium malaysiense strain MUSC 273 Contig 11,
whole genome shotgun sequence; 746242072; NZ_,
JTDI01000011.1
3309; Paenibacillus sp. IHB B 3415 contig_069, whole
genome shotgun sequence; 746258261; NZ_JUEI01000069.1
3310; Novosphingobium subtenaneum strain DSM 12447 NJ75_
contig000013, whole genome shotgun sequence; 746288194;
NZ_JRVC01000013.1
3311; Pandoraea sputorum strain DSM 21091, complete
genome; 749204399; NZ_CP010431.1
3312; Xanthomonas cannabis pv. cannabis strain NCPPB 3753
contig_67, whole genome shotgun sequence; 746366822;
NZ_JSZF01000067.1
3313; Xanthomonas arboricola pv. pruni MAFF 301420 strain
MAFF301420, whole genome shotgun sequence; 759376814;
NZ_BAVC01000017.1
3314; Xanthomonas arboricola pv. celebensis strain
NCPPB 1630 scf 4910810.1, whole genome shotgun
sequence; 746486416; NZ_KL638873.1
3315; Xanthomonas arboricola pv. celebensis strain NCPPB
1832 scf 23466_141.1, whole genome shotgun sequence;
746494072; NZ_KL638866.1
3316; Xanthomonas cannabis pv. cannabis strain NCPPB
2877 contig_94, whole genome shotgun sequence;
746532813; NZ_JSZE01000094.1
3317; Sphingopyxis fribergensis strain Kp5.2, complete
genome; 749188513; NZ_CP009122.1
3318; Sphingopyxis fribergensis strain Kp5.2, complete
genome; 749188513; NZ_CP009122.1
3319; Sphingopyxis fribergensis strain Kp5.2, complete
genome; 749188513; NZ_CP009122.1
3320; Xanthomonas phaseoli pv. phaseoli strain NCPPB 557
scf 22337_104.contig_l, whole genome shotgun sequence;
821373081; NZ_JWTE02000036.1
3321; Corynebacterium minutissimum strain ATCC 23348
Ordered_Contig_015, whole genome shotgun sequence;
746717390; NZ_JSEF01000015.1
3322; Hassallia byssoidea VB512170 scaffold_0, whole
genome shotgun sequence; 748181452; NZ_JTCM01000043.1
3323; Xanthomonas arboricola pv. corylina str. NCCB 100457
Contig50, whole genome shotgun sequence; 507418017;
NZ_APMC02000050.1
3324; Paenibacillus sonchi X19-5 S5_contig01138, whole
genome shotgun sequence; 484099183; NZ_AJTY01001072.1
3325; Pedobacter sp. BAL39 1103467000492, whole genome
shotgun sequence; 149277373; NZ_ABCM01000005.1
3326; Paenibacillus sp. FSL R7-0273, complete genome;
749302091; NZ_CP009283.1
3327; Amycolatopsis decaplanina DSM 44594 Contig0055, whole
genome shotgun sequence; 458848256; NZ_AOHO01000055.1
3328; Rhodanobacter thiooxydans LCS2 contig057, whole
genome shotgun sequence; 389809081; NZ_AJXW01000057.1
3329; Bacillus sp. REN51N contig 2, whole genome shotgun
sequence; 748816024; NZ_JXAB01000002.1
3330; Paenibacillus polymyxa strain Sb3-1, complete genome;
749204146; NZ_CP010268.1
3331; Citrobacter pasteurii strain CIP 55.13, whole genome
shotgun sequence; 749611130; NZ_CDHL01000044.1
3332; Klebsiella pneumoniae CCHB01000016, whole genome
shotgun sequence; 749639368; NZ_CCHB01000016.1
3333; Streptomonospora alba strain YIM 90003 contig 9, whole
genome shotgun sequence; 749673329; NZ_JR0001000009.1
3334; Actinobaculum sp. oral taxon 183 str. F0552 Scaffold1,
whole genome shotgun sequence; 545327174; NZ_KE951406.1
3335; Actinobaculum sp. oral taxon 183 str. F0552 Scaffold15,
whole genome shotgun sequence; 545327527; NZ_KE951412.1
3336; Rubidibacter lacunae KORDI 51-2 KR5 l_contig00121,
whole genome shotgun sequence; 550281965;
NZ_ASSJ01000070.1
3337; Nocardiopsis chromatogenes YIM 90109 contig_93,
whole genome shotgun sequence; 484026206;
NZ_ANBH01000093.1
3338; Streptomyces auratus AGR0001 Scaffoldl, whole
genome shotgun sequence; 398790069; NZ_JH725387.1
3339; Gorillibacterium massiliense strain G5, whole genome
shotgun sequence; 750677319; NZ_CBQR020000171.1
3340; Mesorhizobium sp. 0RS3324, whole genome shotgun
sequence; 751265275; NZ_CCMY01000220.1
3341; Mesorhizobium plurifarium, whole genome shotgun
sequence; 751280166; NZ_CCNB01000034.1
3342; Mesorhizobium sp. SOD10, whole genome shotgun
sequence; 751285871; NZ_CCNA01000001.1
3343; Mesorhizobium plurifarium, whole genome shotgun
sequence; 751292755; NZ_CCNE01000004.1
3344; Mesorhizobium plurifarium, whole genome shotgun
sequence; 751299847; NZ_CCMZ01000015.1
3345; Tolypothrix campylonemoides VB511288 scaffold_0,
whole genome shotgun sequence; 751565075;
NZ_JXCB01000004.1
3346; Jeotgalibacillus campisalis strain SF-57 contig00001,
whole genome shotgun sequence; 751586078;
NZ_ARR01000001.1
3347; Cohnella kolymensis strain VKM B-2846 B2846_22,
whole genome shotgun sequence; 751596254;
NZ_JXAL01000022.1
3348; Jeotgalibacillus soli strain P9 contig00009, whole
genome shotgun sequence; 751619763; NZ_JXRP01000009.1
3349; Burkholderia pseudomallei MSHR4000 scaffold1, whole
genome shotgun sequence; 752517538; NZ_KN323041.1
3350; Burkholderia pseudomallei MSHR4303 scaffold1, whole
genome shotgun sequence; 752519380; NZ_KN323039.1
3351; Burkholderia pseudomallei MSHR4300 scaffold1, whole
genome shotgun sequence; 752522535; NZ_KN322998.1
3352; Burkholderia pseudomallei MSHR4032 scaffold1, whole
genome shotgun sequence; 752526735; NZ_KN323008.1
3353; Geobacter uraniireducens Rf4, complete genome;
148262085; NC_009483.1
3354; Saccharothrix espanaensis DSM 44229 complete
genome; 433601838; NC_019673.1
3355; Mycobacterium sinense strain JDM601, complete
genome; 333988640; NC_015576.1
3356; Sphingomonas wittichii RW1, complete genome;
148552929; NC_009511.1
3357; Sphingopyxis alaskensis RB2256, complete genome;
103485498; NC_008048.1
3358; Sphingopyxis alaskensis RB2256, complete genome;
103485498; NC_008048.1
3359; Synechococcus sp. PCC 6312, complete genome;
427711179; NC_019680.1
3360; Caulobacter sp. K31, complete genome; 167643973;
NC_010338.1
3361; Tistrella mobilis KA081020-065 plasmid pTM1,
complete sequence; 442559580; NC_017957.2
3362; Stackebrandtia nassauensis DSM 44728, complete
genome; 291297538; NC_013947.1
3363; Magnetospirillum gryphiswaldense MSR-1 v2,
complete genome; 568144401; NC_023065.1
3364; Asticcacaulis excentricus CB 48 chromosome 1,
complete sequence; 315497051; NC_014816.1
3365; Emticicia oligotrophica DSM 17448, complete genome;
408671769; NC_018748.1
3366; Clostridium beijerinckii strain NCIMB 14988 genome;
754484184; NZ_CP010086.1
3367; Desulfocapsa sulfexigens DSM 10523, complete
genome; 451945650; NC_020304.1
3368; Gallionella capsifeniformans ES-2, complete genome;
302877245; NC_014394.1
3369; Paenibacillus sp. FSL P4-0081, complete genome;
754777894; NZ_CP009280.1
3370; Streptomyces sp. NBRC 110027, whole genome shotgun
sequence; 754788309; NZ_BBNO01000002.1
3371; Streptomyces sp. NBRC 110027, whole genome shotgun
sequence; 754796661; NZ_BBNO01000008.1
3372; Paenibacillus sp. FSL R7-0331, complete genome;
754821094; NZ_CP009284.1
3373; Paenibacillus camerounensis strain G4, whole genome
shotgun sequence; 754841195; NZ_CCDG010000069.1
3374; Paenibacillus borealis strain DSM 13188, complete
genome; 754859657; NZ_CP009285.1
3375; Paenibacillus sp. FSL R5-0912, complete genome;
754884871; NZ_CP009282.1
3376; Legionella pneumophila serogroup 1 strain TUM 13948,
whole genome shotgun sequence; 754875479; NZ_
BAYQ01000013.1
3377; Nocardiopsis sp. TP-A0876 strain NBRC 110039, whole
genome shotgun sequence; 754924215; NZ_BAZE01000001.1
3378; Streptacidiphilus neutrinimicus strain NBRC 100921,
whole genome shotgun sequence; 755016073;
NZ_BBP001000030.1
3379; Sanguibacter keddieii DSM 10542, complete genome;
269793358; NC_013521.1
3380; Streptacidiphilus jiangxiensis strain NBRC 100920, whole
genome shotgun sequence; 755108320; NZ_BBPN01000056.1
3381; Mesorhizobium sp. ORS3359, whole genome shotgun
sequence; 756828038; NZ_CCNC01000143.1
3382; Streptomyces rimosus strain R6-500MV9-R8 contig021,
whole genome shotgun sequence; 757577710;
NZ_JMGY01000021.1
3383; Burkholderia pseudomallei Bp22 chromosome I, whole
genome shotgun sequence; 485065055; NZ_CM001156.1
3384; Bacillus mycoides strain BHP DJ93.Contig42, whole
genome shotgun sequence; 757763573; NZ_JMQC01000008.1
3385; Aneurinibacillus migulanus strain NCTC 7096 contig_153,
whole genome shotgun sequence; 759007555; NZ_
JYBO01000079.1
3386; Xanthomonas arboricola pv. pruni strain Xap33 contig_
176, whole genome shotgun sequence; 759358038; NZ_
JHUQ01000175.1
3387; Sphingobium sp. Ant17 Contig_45, whole genome
shotgun sequence; 759429528; NZ_JEMV01000036.1
3388; Sphingobium sp. Ant17 Contig_90, whole genome
shotgun sequence; 759431957; NZ_JEMV01000094.1
3389; Bifidobacterium callitrichos DSM 23973 contig4, whole
genome shotgun sequence; 759443001; NZ_JDUV01000004.1
3390; Streptomyces sp. NRRL F-5123 contig24.1, whole
genome shotgun sequence; 671535174; NZ_JOHY01000024.1
3391; Streptomyces vinaceus strain NRRL ISP-5257
contig5.1, whole genome shotgun sequence; 759527818;
NZ_JNYP01000005.1
3392; Burkholderia pseudomallei MSHR1153 chromosome 1,
complete sequence; 759555751; NZ_CP009271.1
3393; Burkholderia thailandensis MSMB43 Scaffold3, whole
genome shotgun sequence; 424903876; NZ_JH692063.1
3394; Pseudomonas sp. HMP271 Pseudomonas HMP271_
contig_7, whole genome shotgun sequence; 759578528;
NZ_JMFZ01000007.1
3395; Stenotrophomonas maltophilia strain B418 Contig_4_,
whole genome shotgun sequence; 759679095; NZ_
JSXG01000004.1
3396; Kitasatospora sp. MBT66 scaffold3, whole genome
shotgun sequence; 759755931; NZ_JAIY01000003.1
3397; Streptomyces bingchenggensis BCW-1, complete
genome; 374982757; NC_016582.1
3398; Streptomyces glaucescens strain GLA.0, complete
genome; 759802587; NZ_CP009438.1
3399; Streptomyces glaucescens strain GLA.0, complete
genome; 759802587; NZ_CP009438.1
3400; Actinomyces israelii DSM 43320 O145DRAFT_
scaffold00014.14_C, whole genome shotgun sequence;
759875025; NZ_JONS01000016.1
3401; Rubrivivax gelatinosus IL144 DNA, complete genome;
383755859; NC_017075.1
3402; Clostridium butyricum strain HM-68 Contig83, whole
genome shotgun sequence; 760273878; NZ_JXBT01000001.1
3403; Novosphingobium sp. P6W scaffold3, whole genome
shotgun sequence; 763092879; NZ_JXZE01000003.1
3404; Streptomyces fulvissimus DSM 40593, complete
genome; 488607535; NC_021177.1
3405; Novosphingobium sp. P6W scaffold9, whole genome
shotgun sequence; 763095630; NZ_JXZE01000009.1
3406; Bifidobacterium reuteri DSM 23975 contig4, whole
genome shotgun sequence; 763216595; NZ_JDUW01000004.1
3407; Sphingomonas hengshuiensis strain WHSC-8, complete
genome; 764364074; NZ_CP010836.1
3408; Sphingomonas hengshuiensis strain WHSC-8, complete
genome; 764364074; NZ_CP010836.1
3409; Burkholderia pseudomallei strain QCMRI_BP13 Contig 7,
whole genome shotgun sequence; 764427571; NZ_JYBH01000021.1
3410; Streptomyces natalensis ATCC 27448 Scaffold_33, whole
genome shotgun sequence; 764439507; NZ_JRKI01000027.1
3411; Streptomyces griseus strain S4-7 contig113, whole
genome shotgun sequence; 764464761; NZ_JYBE01000113.1
3412; Streptomyces cyaneogriseus subsp. noncyanogenus strain
NMWT 1, complete genome; 764487836; NZ_CP010849.1
3413; Bacillus subtilis subsp. spizizenii RFWG1A4 contig00010,
whole genome shotgun sequence; 764657375; NZ_AJHM01000010.1
3414; Mastigocladus laminosus UU774 scaffold_22, whole
genome shotgun sequence; 764671177; NZ_JX1101000139.1
3415; Bacillus subtilis subsp. spizizenii RFWG5B15 contig00010,
whole genome shotgun sequence; 764677272; NZ_AJHO01000010.1
3416; Streptomyces iranensis genome assembly Siranensis,
scaffold SCAF00002; 765016627; NZ_LK022849.1
3417; Risungbinella massiliensis strain GD1, whole genome
shotgun sequence; 765315585; NZ_LN812103.1
3418; Risungbinella massiliensis strain GD1, whole genome
shotgun sequence; 765315585; NZ_LN812103.1
3419; Paenibacillus tenae strain NRRL B-30644 contig00007,
whole genome shotgun sequence; 765319397; NZ_JTHP01000007.1
3420; Sphingobium sp. YBL2, complete genome; 765344939;
NZ_CP010954.1
3421; Sphingobium sp. YBL2, complete genome; 765344939;
NZ_CP010954.1
3422; Streptococcus suis strain LS5J, whole genome
shotgun sequence; 765394696; NZ_CEEZ01000028.1
3423; Bacillus mycoides strain 11kri323 LG56_082, whole
genome shotgun sequence; 765533368; NZ_JYCJ01000082.1
3424; Streptococcus suis strain LS8F, whole genome shotgun
sequence; 766589647; NZ_CEHJ01000007.1
3425; Streptococcus suis strain L58I, whole genome shotgun
sequence; 766595491; NZ_CEHM01000004.1
3426; Paenibacillus polymyxa strain NRRL B-30509 contig00003,
whole genome shotgun sequence; 766607514; NZ_JTHO01000003.1
3427; Thalassospira sp. HJNODE_2, whole genome shotgun
sequence; 766668420; NZ_JYII01000010.1
3428; Paenibacillus sp. IHBB 10380, complete genome;
767005659; NZ_CP010976.1
3429; Frankia sp. CpI1-S FF36_scaffold_9.10, whole genome
shotgun sequence; 768715243; NZ_JYFN01000010.1
3430; Streptococcus suis strain B28P, whole genome shotgun
sequence; 769231516; NZ_CDTB01000010.1
3431; Lechevalieria aerocolonigenes strain NRRL B-16140
contig11.3, whole genome shotgun sequence; 772744565;
NZ_JYJG01000059.1
3432; Streptomyces sp. NRRL F-4428 contig40.2, whole genome
shotgun sequence; 772774737; NZ_JYJI01000131.1
3433; Bacterium endosymbiont of Mortierella elongata FMR23-6,
whole genome shotgun sequence; 779889750; NZ_DF850521.1
3434; Bacterium endosymbiont of Mortierella elongata FMR23-6,
whole genome shotgun sequence; 779889750; NZ_DF850521.1
3435; Streptomyces sp. FxanaA7 F611DRAFT_scaffold00041.41_
C, whole genome shotgun sequence; 780340655;
NZ_LACL01000054.1
3436; Burkholderia oklahomensis C6786 chromosome I,
complete sequence; 780352952; NZ_CP009555.1
3437; Burkholderia pseudomallei MSHR2543 chromosome I,
complete sequence; 782642065; NZ_CP009478.1
3438; Burkholderia thailandensis 34 chromosome 1, complete
sequence; 782674607; NZ_CP010017.1
3439; Streptomyces rubellomurinus strain ATCC 31215 contig-63,
whole genome shotgun sequence; 783211546;
3440; Burkholderia pseudomallei strain MSHR5107 Contig_3,
whole genome shotgun sequence; 785595141; NZ_JZXP01000013.1
3441; Elstera litoralis strain Dia-1 c21, whole genome shotgun
sequence; 788026242; NZ_LAJY01000021.1
3442; Frankia sp. DC12 FraDC12DRAFT_scaffold1.1, whole
genome shotgun sequence; 797224947; NZ_KQ031391.1
3443; Sphingomonas sp. SRS2 contig40, whole genome shotgun
sequence; 806905234; NZ_LARW01000040.1
3444; Bacillus sp. UMTAT18 contig000011, whole genome
shotgun sequence; 806951735; NZ_JSFD01000011.1
3445; Paenibacillus wulumuqiensis strain Y24 Scaffold4, whole
genome shotgun sequence; 808051893; NZ_KQ040793.1
3446; Bacillus endophyticus strain Hbe603, complete genome;
890672806; NZ_CP011974.1
3447; Paenibacillus algorifonticola strain XJ259 Scaffold20_1,
whole genome shotgun sequence; 808072221; NZ_LAQ001000025.1
3448; Streptomyces sp. MBT28 contig_50, whole genome
shotgun sequence; 808090008; NZ_LARV01000050.1
3449; Mycobacterium sp. UM_Kg27 contig000002, whole
genome shotgun sequence; 809025315; NZ_JRMM01000002.1
3450; Mycobacterium sp. UM_Kg1 contig000164, whole
genome shotgun sequence; 809073490; NZ_JRMK01000164.1
3451; Xanthomonas campesiiis strain 17, complete genome;
810489403; NZ_CP011256.1
3452; Spirosoma radiotolerans strain DG5A, complete genome;
817524426; NZ_CP010429.1
3453; Allosalinactinospora lopnorensis strain CA15-2 contig00044,
whole genome shotgun sequence; 815863894; NZ_LAJC01000044.1
3454; Bacillus sp. SA1-12 scf7180000003378, whole genome
shotgun sequence; 817541164; NZ_LATZ01000026.1
3455; Streptomyces xiamenensis strain 318, complete genome;
921170702; NZ_CP009922.2
3456; Streptomyces xiamenensis strain 318, complete genome;
921170702; NZ_CP009922.2
3457; Altererythrobacter atlanticus strain 26DY36, complete
genome; 927872504; NZ_JZKH01000064.1 NZ_CP011452.2
3458; Streptomyces lydicus A02, complete genome; 822214995;
NZ_CP007699.1
3459; Streptomyces lydicus A02, complete genome; 822214995;
NZ_CP007699.1
3460; Streptomyces lydicus A02, complete genome; 822214995;
NZ_CP007699.1
3461; Bacillus cereus strain B4147 NODES, whole genome
shotgun sequence; 822530609; NZ_LCYN01000004.1
3462; Xanthomonas pisi DSM 18956 Contig_28, whole
genome shotgun sequence; 822535978; NZ_JPLE01000028.1
3463; Erythrobacter luteus strain KA37 contig 1, whole genome
shotgun sequence; 822631216; NZ_LBHB01000001.1
3464; Erythrobacter marinus strain KCTC 23554
KCTC23554_C3, whole genome shotgun sequence; 829088381;
NZ_LDCP01000003.1
3465; Streptomyces leeuwenhoekii strain C34(2013) c34_
sequence_0041, whole genome shotgun sequence;
657295264; NZ_AZSD01000040.1
3466; Streptomyces leeuwenhoekii strain C34(2013) c34_
sequence_0012, whole genome shotgun sequence;
657294764; NZ_AZSD01000012.1
3467; Xanthomonas arboricola strain CFBP 7634 Xaijug-
CFBP7634-G11, whole genome shotgun sequence;
825139250; NZ_JZEH01000001.1
3468; Xanthomonas arboricola strain CFBP 7651 Xaijug-
CFBP7651-G11, whole genome shotgun
sequence; 825156557; NZ_JZEI01000001.1
3469; Luteimonas sp. FCS-9 scf7180000000225, whole
genome shotgun sequence; 825314716; NZ_LASZ01000002.1
3470; Luteimonas sp. FCS-9 scf7180000000226, whole
genome shotgun sequence; 825314728; NZ_LASZ01000003.1
3471; Streptomyces sp. KE1 Contigll, whole genome
shotgun sequence; 825353621; NZ_LAYX01000011.1
3472; Frankia coriariae strain BMG5.1 scaffold41.42, whole
genome shotgun sequence; 827465632; NZ__MO01000042.1
3473; Erythrobacter marinus strain KCTC 23554 KCTC23554_C3,
whole genome shotgun sequence; 829088381;
NZ_LDCP01000003.1
3474; Alistipes sp. ZOR0009 L990_140, whole genome
shotgun sequence; 835319962; NZ_JTLD01000119.1
3475; Streptomyces sp. M10 Scaffold2, whole genome
shotgun sequence; 835355240; NZ_KN549147.1
3476; Bacillus aryabhattai strain T61 Scaffold1, whole
genome shotgun sequence; 836596561; NZ_KQ087173.1
3477; Croceicoccus naphthovorans strain PQ-2, complete
genome; 836676868; NZ_CP011770.1
3478; Paenibacillus sp. TCA20, whole genome shotgun sequence;
843088522; NZ_BBIW01000001.1
3479; Bacillus circulans strain RIT379 contig11, whole genome
shotgun sequence; 844809159; NZ_LDPH01000011.1
3480; Omithinibacillus califomiensis strain DSM 16628 contig_22,
whole genome shotgun sequence; 849059098; NZ_LDUE01000022.1
3481; Bacillus pseudalcaliphilus strain DSM 8725 super11, whole
genome shotgun sequence; 849078078; NZ_LFJO01000006.1
3482; Bacillus aryabhattai strain LK25 16, whole genome
shotgun sequence; 850356871; NZ_LDWN01000016.1
3483; Methanobacterium sp. SMA-27 DL91DRAFT_unitig_0_
quiver. l_C, whole genome shotgun sequence; 851351157;
NZ_JQLY01000001.1
3484; Cellulomonas sp. A375-1 contig_129, whole genome
shotgun sequence; 856992287; NZ_LFKW01000127.1
3485; Bacillus cereus strain RIMV BC 126 212, whole genome
shotgun sequence; 872696015; NZ_LABO01000035.1
3486; Streptomyces leeuwenhoekii strain C58 contig69, whole
genome shotgun sequence; 873282617; NZ_LFEH01000068.1
3487; Streptomyces leeuwenhoekii strain C58 contig126, whole
genome shotgun sequence; 873282818; NZ_LFEH01000123.1
3488; Sphingomonas sp. MEA3-1 contig00021, whole genome
shotgun sequence; 873296042; NZ_LECE01000021.1
3489; Sphingomonas sp. MEA3-1 contig00040, whole genome
shotgun sequence; 873296160; NZ_LECE01000040.1
3490; Sphingomonas sp. MEA3-1 contig00071, whole genome
shotgun sequence; 873296295; NZ_LECE01000071.1
3491; Bacillus sp. 220_BSPC 1447_75439_1072255, whole
genome shotgun sequence; 880954155; NZ_JVPL01000109.1
3492; Bacillus sp. 522_BSPC 2470_72498_1083579_
594__ . . . _522_, whole genome shotgun sequence; 880997761;
NZ_JVDT01000118.1
3493; Nostoc sp. PCC 7107, complete genome; 427705465;
NC_019676.1
3494; Streptomyces decoyicus strain NRRL ISP-5087 P056_
Doro1_scaffold78, whole genome shotgun sequence;
662133033; NZ_KL570321.1
3495; Streptomyces varsoviensis strain NRRL B-3589 contig2.1,
whole genome shotgun sequence; 664348063; NZ_JOFN01000002.1
3496; Scytonema tolypothrichoides VB-61278 scaffold_6, whole
genome shotgun sequence; 890002594; NZ_JXCA01000005.1
3497; Erythrobacter atlanticus strain s21-N3, complete genome;
890444402; NZ_CP011310.1
3498; Sphingobium yanoikuyae strain SHJ scaffold2, whole
genome shotgun sequence; 893711333; NZ_KQ235984.1
3499; Sphingobium yanoikuyae strain SHJ scaffold12, whole
genome shotgun sequence; 893711343; NZ_KQ235994.1
3500; Sphingobium yanoikuyae strain SHJ scaffold33, whole
genome shotgun sequence; 893711364; NZ_KQ236015.1
3501; Sphingobium yanoikuyae strain SHJ scaffold47, whole
genome shotgun sequence; 893711378; NZ_KQ236029.1
3502; Stenotrophomonas maltophilia strain 544_SMAL
1161_223966_2976806_599__ . . . _882_, whole genome shotgun
sequence; 896492362; NZ_JVCU01000107.1
3503; Stenotrophomonas maltophilia strain 517_SMAL
472_405557_4951990_20_ . . . _115_, whole genome shotgun
sequence; 896506125; NZ_JVDZ01000045.1
3504; Stenotrophomonas maltophilia strain 131_SMAL
1126_236170_8501292_717__ . . . _1018_, whole genome shotgun
sequence; 896520167; NZ_JVUI01000038.1
3505; Stenotrophomonas maltophilia strain 419_SMAL
707_128228_1961615_4__642__523_, whole genome shotgun
sequence; 896535166; NZ_JVHW01000017.1
3506; Stenotrophomonas maltophilia strain 179_SMAL
631_468538_7028045_522__ . . . _127_, whole genome shotgun
sequence; 896555871; NZ_JVRD01000056.1
3507; Stenotrophomonas maltophilia strain 951_SMAL 71_
125859_2268311, whole genome shotgun sequence; 896567682;
NZ_JUMH01000022.1
3508; Stenotrophomonas maltophilia strain 22_SMAL
361_494818_13518495_244__194__203_, whole genome shotgun
sequence; 896599318; NZ_JVPM01000019.1
3509; Streptococcus pseudopneumoniae strain 445_SPSE
347_91401_2272315_318__ . . . _319_, whole genome shotgun
sequence; 896667361; NZ_JVGV01000030.1
3510; Streptomyces sp. SBT349 scaffold307_size9018, whole
genome shotgun sequence; 898301838; NZ_LAVK01000307.1
3511; Kitasatospora sp. MY 5-36 Contig_703_, whole genome
shotgun sequence; 902792184; NZ_LFVW01000692.1
3512; Streptomyces caatingaensis strain CMAA 1322 contig02,
whole genome shotgun sequence; 906344334; NZ_LFXA01000002.1
3513; Streptomyces caatingaensis strain CMAA 1322 contig02,
whole genome shotgun sequence; 906344334; NZ_LFXA01000002.1
3514; Streptomyces caatingaensis strain CMAA 1322 contig07,
whole genome shotgun sequence; 906344339; NZ_LFXA01000007.1
3515; Streptomyces caatingaensis strain CMAA 1322 contig09,
whole genome shotgun sequence; 906344341; NZ_LFXA01000009.1
3516; Xanthomonas arboricola 3004 contig00003, whole genome
shotgun sequence; 640500871; NZ_AZQY01000003.1
3517; Candidatus Halobonum tynellensis G22 contig00002, whole
genome shotgun sequence; 557371823; NZ_ASGZ01000002.1
3518; Streptomyces wadayamensis strain A23 LGO_A23_A57_
CO0257, whole genome shotgun sequence; 910050821;
NZ_JHDU01000034.1
3519; Bacillus weihenstephanensis strain JAS 83/3 Bw_
JAS-83/3_contig00005, whole genome shotgun sequence;
910095435; NZ_JNLY01000005.1
3520; Silvibacterium bohemicum strain S15 contig_3, whole
genome shotgun sequence; 910257956; NZ_LBHJ01000003.1
3521; Silvibacterium bohemicum strain S15 contig_3, whole
genome shotgun sequence; 910257956; NZ_LBHJ01000003.1
3522; Silvibacterium bohemicum strain S15 contig_30, whole
genome shotgun sequence; 910257973; NZ_LBHJ01000020.1
3523; Streptococcus pneumoniae strain 37, whole genome
shotgun sequence; 912648153; NZ_CKHR01000004.1
3524; Streptococcus pneumoniae strain 37, whole genome
shotgun sequence; 912676034; NZ_CMPZ01000004.1
3525; Streptomyces fradiae strain ATCC 19609 contig0008,
whole genome shotgun sequence; 759752221; NZ_JNAD01000008.1
3526; Streptomyces sp. CNS654 CD02DRAFT_
scaffold00023.23S, whole genome shotgun sequence; 695856316;
NZ_JNLT01000024.1
3527; Streptomyces griseus subsp. rhodochrous strain NRRL
B-2931 contig3.1, whole genome shotgun sequence; 664191782;
NZ_JOFE01000003.1
3528; Streptomyces sp. NRRL F-2202 contig25.1, whole
genome shotgun sequence; 695860443; NZ_JOIH01000025.1
3529; Streptomyces puipeochromogenes strain NRRL B-3012
contig5.1, whole genome shotgun sequence; 663242068;
NZ_JODK01000005.1
3530; Streptomyces griseus subsp. rhodochrous strain NRRL
B-2932 contig37.1, whole genome shotgun sequence; 664207653;
NZ_JOFF01000037.1
3531; Streptomyces sp. NRRL F-5702 contig3.1, whole
genome shotgun sequence; 664537198; NZ_JOHD01000003.1
3532; Streptomyces albus subsp. albus strain NRRL B-2445
contig1.1, whole genome shotgun sequence; 664084661;
NZ_JOED01000001.1
3533; Streptomyces baamensis strain NRRL B-2842 P144_
Doro1_scaffold6, whole genome shotgun sequence; 662129456;
NZ_KL573544.1
3534; Streptomyces sp. NRRL F-3218 contig19.1, whole
genome shotgun sequence; 664170107; NZ_JOIP01000019.1
3535; Streptomyces albus subsp. albus strain NRRL B-2445
contig1.1, whole genome shotgun sequence; 664084661;
NZ_JOED01000001.1
3536; Streptomyces albus subsp. albus strain NRRL B-16041
contig26.1, whole genome shotgun sequence; 695869320;
NZ_JNWW01000026.1
3537; Streptomyces albus subsp. albus strain NRRL B-16041
contig28.1, whole genome shotgun sequence; 695870063;
NZ_JNWW01000028.1
3538; Streptomyces peucetius strain NRRL WC-3868 contig49.1,
whole genome shotgun sequence; 665671804; NZ_JOCK01000052.1
3539; Erythrobacter citreus LAMA 915 Contig13, whole
genome shotgun sequence; 914607448; NZ_JYNE01000028.1
3540; Bacillus flexus strain Riq5 contig_32, whole genome
shotgun sequence; 914730676; NZ_LFQJ01000032.1
3541; Xylanimonas cellulosilytica DSM 15894, complete
genome; 269954810; NC_013530.1
3542; Streptomyces sp. Mg1, complete genome; 847063800;
NZ_CP011664.1
3543; Streptomyces sviceus ATCC 29083 chromosome, whole
genome shotgun sequence; 297196766; NZ_CM000951.1
3544; Burkholderia pseudomallei strain MSHR0169 Contig_2,
whole genome shotgun sequence; 915621003; NZ_LGKL01000002.1
3545; Burkholderia pseudomallei strain E25, whole genome
shotgun sequence; 915671105; NZ_CSLP01000001.1
3546; Streptomyces xinghaiensis S187 contig_1763_1, whole
genome shotgun sequence; 485454803; NZ_AFRP01001656.1
3547; Streptomyces sp. W007 contig00241, whole genome
shotgun sequence; 365866490; NZ_AGSW01000226.1
3548; Streptomyces violaceusniger Tu 4113, complete genome;
345007964; NC_015957.1
3549; Streptomyces mobaraensis NBRC 13819 = DSM
40847 contig024, whole genome shotgun sequence; 458977979;
NZ_AORZ01000024.1
3550; Streptomyces mobaraensis NBRC 13819 = DSM 40847
contig079, whole genome shotgun sequence;
458984960; NZ_AORZ01000079.1
3551; Actinokineospora enzanensis DSM 44649 C503DRAFT_
scaffold00014.14, whole genome shotgun sequence;
484005069; NZ_KB894416.1
3552; Streptomyces sp. FXJ7.023 Contig10, whole genome
shotgun sequence; 510871397; NZ_APIV01000010.1
3553; Nocardia transvalensis NBRC 15921, whole genome
shotgun sequence; 485125031; NZ_BAGL01000055.1
3554; Caulobacter sp. URHA0033 H963DRAFT_
scaffold00023.23_C, whole genome shotgun sequence; 654573246;
NZ_AUEO01000025.1
3555; Gloeobacter kilaueensis JS1, complete genome;
554634310; NC_022600.1
3556; Actinomadura oligospora ATCC 43269
P696DRAFT_scaffold00008.8_C, whole genome shotgun sequence;
651281457; NZ_JADG01000010.1
3557; Actinomadura oligospora ATCC 43269
P696DRAFT_scaffold00008.8_C, whole genome shotgun sequence;
651281457; NZ_JADG01000010.1
3558; Streptomyces sp. Tu 6176 scaffold00003, whole genome
shotgun sequence; 740044478; NZ_KK106990.1
3559; Sphingomonas paucimobilis strain EPA505 contig000027,
whole genome shotgun sequence; 739630357; NZ_JFYY01000027.1
3560; Paenibacillus sp. UNC217MF BP95DRAFT_
scaffold00011.11_C, whole genome shotgun sequence;
655084059; NZ_JMLT01000016.1
3561; Hyphomonas chukchiensis strain BH-BN04-4 contig29,
whole genome shotgun sequence; 736736050; NZ_AWFG01000029.1
3562; Fusobacterium necrophorum BFTR-2 contig0075, whole
genome shotgun sequence; 737951550; NZ_JAAG01000075.1
3563; Streptomyces sp. NRRL F-5917 contig68.1, whole genome
shotgun sequence; 663414324; NZ_JOHQ01000068.1
3564; Streptomyces sp. NRRL F-5639 contig31.1, whole genome
shotgun sequence; 664512262; NZ_JOGK01000031.1
3565; Streptomyces sp. NRRL F-5639 contig75.1, whole genome
shotgun sequence; 664515060; NZ_JOGK01000075.1
3566; Streptomyces megasporus strain NRRL B-16372 contig19.1,
whole genome shotgun sequence; 671525382; NZ_JODL01000019.1
3567; Streptomyces albus subsp. albus strain NRRL B-1811 contig32.1,
whole genome shotgun sequence; 665618015; NZ_JODR01000032.1
3568; Streptomyces albus subsp. albus strain NRRL B-1811 contig49.1,
whole genome shotgun sequence; 665618560; NZ_JODR01000049.1
3569; Streptomyces griseus subsp. griseus strain NRRL WC-3480
contig2.1, whole genome shotgun sequence; 664166765;
NZ_JOBR01000002.1
3570; Streptomyces griseorubens strain JSD-1 scaffold1, whole
genome shotgun sequence; 739792456; NZ_KL503830.1
3571; Streptomyces achromogenes subsp. achromogenes strain
NRRL B-2120 contig2.1, whole genome shotgun sequence;
664063830; NZ_JODT01000002.1
3572; Nocardia sp. NRRL WC-3656 contig2.1, whole genome
shotgun sequence; 663737675; NZ_JOJF01000002.1
3573; Streptomyces sp. NRRL S-337 contig31.1, whole genome
shotgun sequence; 664275807; NZ_JOIX01000031.1
3574; Streptomyces sp. NRRL S-337 contig41.1, whole genome
shotgun sequence; 664277815; NZ_JOIX01000041.1
3575; Streptomyces albus subsp. albus strain NRRL B-2362 contig48.1,
whole genome shotgun sequence; 739761647; NZ_JODZ01000048.1
3576; Streptomyces ruber strain NRRL ISP-5378 contig2.1, whole
genome shotgun sequence; 665674644; NZ_JOAQ01000002.1
3577; Streptomyces lavenduligriseus strain NRRL ISP-5487 contig2.1,
whole genome shotgun sequence; 664244706; NZ_JOBD01000002.1
3578; Streptomyces sp. NRRL S-920 contig36.1, whole genome
shotgun sequence; 664256887; NZ_JODF01000036.1
3579; Streptomyces sp. NRRL S-1448 contig134.1, whole
genome shotgun sequence; 663421576; NZ_JOGE01000134.1
3580; Streptomyces bicolor strain NRRL B-3897 contig42.1,
whole genome shotgun sequence; 671498318; NZ_JOFRO1000042.1
3581; Streptomyces sp. NRRL WC-3719 contig52.1, whole
genome shotgun sequence; 665530468; NZ_JOCD01000052.1
3582; Streptomyces sp. NRRL WC-3719 contig152.1, whole
genome shotgun sequence; 665536304; NZ_JOCD01000152.1
3583; Streptomyces sp. NRRL WC-3641 P206_Dorol_scaffold18,
whole genome shotgun sequence; 664607641; NZ_KL579016.1
3584; Streptomyces sp. NRRL B-1347 contig19.1, whole genome
shotgun sequence; 664141438; NZ_JOJM01000019.1
3585; Streptomyces toyocaensis strain NRRL 15009 contig00064,
whole genome shotgun sequence; 740092143; NZ_JFCB01000064.1
3586; Streptomyces natalensis strain NRRL B-5314 P055_
Dorol_scaffold13, whole genome shotgun sequence; 662108422;
NZ_KL570019.1
3587; Sphingobium yanoikuyae strain B1 contig000002, whole
genome shotgun sequence; 739661773; NZ_JGVR01000002.1
3588; Kibdelosporangium aridum subsp. largum strain NRRL
B-24462 contig91.5, whole genome shotgun sequence;
703243990; NZ_JNYM01001430.1
3589; Streptomyces ruber strain NRRL ISP-5378 contig2.1, whole
genome shotgun sequence; 665674644; NZ_JOAQ01000002.1
3590; Kutzneria albida DSM 43870, complete genome;
754862786; NZ_CP007155.1
3591; Streptomyces aurantiacus JA 4570 Seq28, whole genome
shotgun sequence; 514916412; NZ_AOPZ01000028.1
3592; Rothia dentocariosa strain C6B contig 5, whole genome
shotgun sequence; 739372122; NZJOHE01000003.1
3593; Xanthomonas cannabis pv. phaseoli strain Nyagatare scf
52938_7, whole genome shotgun sequence; 835885587;
NZ_KN265462.1
3594; Novosphingobium malaysiense strain MUSC 273 Contig9,
whole genome shotgun sequence; 746241774; NZ_JIDI01000009.1
3595; Novosphingobium subtenaneum strain DSM 12447 NJ75_
contig000028, whole genome shotgun sequence; 746290581;
NZJRVC01000028.1
3596; Jeotgalibacillus malaysiensis strain D5 chromosome,
complete genome; 749182744; NZ_CP009416.1
3597; Microcystis panniformis FACHB-1757, complete
genome; 917764592; NZ_CP011339.1
3598; Streptomyces sp. 769, complete genome; 749181963;
NZ_CP003987.1
3599; Actinoplanes sp. SE50/110, complete genome; 386845069;
NC_017803.1
3600; Salinarimonas rosea DSM 21201 G407DRAFT_
scaffold00021.21_C, whole genome shotgun sequence; 655990125;
NZ_AUBC01000024.1
3601; Methanobacterium arcticum strain M2 EI99DRAFT_
scaffold00005.5_C, whole genome shotgun sequence; 851140085;
NZ_JQKN01000008.1
3602; Allokutzneria albata strain NRRL B-24461 contig22.1,
whole genome shotgun sequence; 663596322; NZ_JOEF01000022.1
3603; Streptomyces olivaceus strain NRRL B-3009 contig20.1,
whole genome shotgun sequence; 664523889; NZ_JOFH01000020.1
3604; Streptomyces sp. NRRL S-118 P205_Doro1_scaffold2,
whole genome shotgun sequence; 664556736; NZ_KL591003.1
3605; Streptomyces sp. NRRL S-118 P205_Doro1_scaffold34,
whole genome shotgun sequence; 664565137; NZ_KL591029.1
3606; Streptomyces luteus strain TRM 45540 Scaffold1, whole
genome shotgun sequence; 759659849; NZ_KN039946.1
3607; Nonomumea candida strain NRRL B-24552 contig8 1, whole
genome shotgun sequence; 759934284; NZ_JOAG01000009.1
3608; Nonomumea candida strain NRRL B-24552 contig19.1, whole
genome shotgun sequence; 759941310; NZ_JOAG01000020.1
3609; Nonomumea candida strain NRRL B-24552 contig27.1, whole
genome shotgun sequence; 759944049; NZ_JOAG01000029.1
3610; Nonomumea candida strain NRRL B-24552 contig28.1, whole
genome shotgun sequence; 759944490; NZ_JOAG01000030.1
3611; Nonomumea candida strain NRRL B-24552 contig42.1, whole
genome shotgun sequence; 759948103; NZ_JOAG01000045.1
3612, Streptacidiphilus melanogenes strain NBRC 103184, whole
genome shotgun sequence; 755032408; NZ_BBPP01000024.1
3613, Streptacidiphilus anmyonensis strain NBRC 103185, whole
genome shotgun sequence; 755077919; NZ_BBPQ01000048.1
3614; Streptomyces nodosus strain ATCC 14899 genome;
759739811; NZ_CP009313.1
3615; Kibdelosporangium sp. MJ126-NF4, whole genome
shotgun sequence; 754819815; NZ_CDME01000002.1
3616; Streptomyces albus strain DSM 41398, complete
genome; 749658562; NZ_CP010519.1
3617; Novosphingobium sp. P6W scaffold17, whole genome
shotgun sequence; 763097360; NZ_JXZE01000017.1
3618; Magnetospirillum gryphiswaldense MSR-1 v2,
complete genome; 568144401; NC_023065.1
3619; Methanobacterium fonnicicum genome assembly DSM1535,
chromosome: chrI; 851114167; NZ_LN515531.1
3620; Streptomyces sp. NRRL B-1568 contig-76, whole
genome shotgun sequence; 799161588; NZ_JZWZ01000076.1
3621; Streptomyces rubellomurinus subsp. indigoferus strain
ATCC 31304 contig-55, whole genome shotgun sequence;
783374270; NZ_JZKG01000056.1
3622; Paenibacillus dauci strain H9 Scaffold3, whole genome
shotgun sequence; 808064534; NZ_KQ040798.1
3623; Allosalinactinospora lopnorensis strain CA15-2 contig00053,
whole genome shotgun sequence; 815864238; NZ_LAJC01000053.1
3624; Jiangella alkaliphila strain KCTC 19222 Scaffold1, whole
genome shotgun sequence; 820820518; NZ_KQ061219.1
3625; Streptomyces natalensis ATCC 27448 Scaffold 46, whole
genome shotgun sequence; 764442321; NZ_JRKI01000041.1
3626; Sphingomonas parapaucimobilis NBRC 15100 BBPI01000030,
whole genome shotgun sequence; 755134941; NZ_BBPI01000030.1
3627; Streptomyces avicenniae strain NRRL B-24776 contig3.1,
whole genome shotgun sequence; 919531973; NZ_JOEK01000003.1
3628; Streptomyces celluloflavus strain NRRL B-2493 contig27.1,
whole genome shotgun sequence; 919546534; NZ_JOEL01000027.1
3629; Streptomyces celluloflavus strain NRRL B-2493 contig60.1,
whole genome shotgun sequence; 919546651; NZ_JOEL01000060.1
3630; Streptomyces celluloflavus strain NRRL B-2493 contig66.1,
whole genome shotgun sequence; 919546672; NZ_JOEL01000066.1
3631; Sphingomonas sp. Y57 scaffold74, whole genome
shotgun sequence; 826051019; NZ_LDE501000074 .1
3632; Xanthomonas arboricola pv. juglandis strain Xaj 417
genome; 920673152; NZ_CP012251.1
3633; Xanthomonas campestris strain CFSAN033089 contig 46,
whole genome shotgun sequence; 920684790;
NZ_LHBW01000046.1
3634; Streptomyces sp. Mg1 supercont1.100, whole genome
shotgun sequence; 254387191; NZ_DS570483.1
3635; Streptomyces sp. HNS054 contig28, whole genome
shotgun sequence; 860547590; NZ_LDZX01000028.1
3636; Streptomyces ahygroscopicus subsp. wuyiensis strain
CK-15 contig3, whole genome shotgun sequence; 921220646;
NZ_JXYI02000059.1
3637; Paenibacillus peoriae strain HS311, complete genome;
922052336; NZ_CP011512.1
3638; Paenibacillus sp. FJAT-27812 scaffold_0, whole
genome shotgun sequence; 922780240; NZ_LIGH01000001.1
3639; Stenotrophomonas maltophilia strain ISMMS2R,
complete genome; 923060045; NZ_CP011306.1
3640; Stenotrophomonas maltophilia strain ISMMS3,
complete genome; 923067758; NZ_CP011010.1
3641; Hapalosiphon sp. MRB220 contig 91, whole genome
shotgun sequence; 923076229; NZ_LIRN01000111.1
3642; Bacillus sp. FJAT-18019 superl, whole genome
shotgun sequence; 924371245; NZ_LITP01000001.1
3643; Stenotrophomonas maltophilia strain B4 contig779, whole
genome shotgun sequence; 924516300; NZ_LDVR01000003.1
3644; Bacillus sp. FJAT-21352 Scaffold 1, whole genome
shotgun sequence; 924654439; NZ_LIUS01000003.1
3645; Sphingopyxis sp. 113P3, complete genome; 924898949;
NZ_CP009452.1
3646; Sphingopyxis sp. 113P3, complete genome; 924898949;
NZ_CP009452.1
3647; Streptomyces sp. CFMR 7 strain CFMR-7, complete
genome; 924911621; NZ_CP011522.1
3648; Bacillus gobiensis strain FJAT-4402 chromosome;
926268043; NZ_CP012600.1
3649; Streptomyces sp. M1V1G1522 P406contig11.1, whole
genome shotgun sequence; 926270045; NZ_LGDF01000013.1
3650; Nocardiopsis sp. NRRL B-16309 P441contig5.1, whole
genome shotgun sequence; 926283036; NZ_LGEC01000103.1
3651; Streptomyces sp. NRRL F-2295 P395contig79.1, whole
genome shotgun sequence; 926288193; NZ_LGCY01000146.1
3652; Streptomyces sp. XY431 P412contig111.1, whole
genome shotgun sequence; 926317398; NZ_LGDO01000015 .1
3653; Streptomyces sp. NRRL F-6492 P446contig3.1, whole
genome shotgun sequence; 926315769; NZ_LGEG01000211.1
3654; Streptomyces sp. NRRL B-1140 P439contig15.1, whole
genome shotgun sequence; 926344107; NZ_LGEA01000058.1
3655; Streptomyces sp. NRRL B-1140 P439contig32.1, whole
genome shotgun sequence; 926344331; NZ_LGEA01000105.1
3656; Streptomyces sp. NRRL F-5755 P309contig48.1, whole
genome shotgun sequence; 926371517; NZ_LGCW01000271.1
3657; Streptomyces sp. NRRL F-5755 P309contig50.1, whole
genome shotgun sequence; 926371520; NZ_LGCW01000274.1
3658; Streptomyces sp. NRRL F-5755 P309contig7.1, whole
genome shotgun sequence; 926371541; NZ_LGCW01000295.1
3659; Saccharothrix sp. NRRL B-16348 P442contig71.1, whole
genome shotgun sequence; 926395199; NZ_LGED01000246.1
3660; Streptomyces sp. WM6378 P402contig63.1, whole
genome shotgun sequence; 926403453; NZ_LGDD01000321.1
3661; Streptomyces sp. WM6378 P402contig63.1, whole
genome shotgun sequence; 926403453; NZ_LGDD01000321.1
3662; Nocardia sp. NRRL S-836 P437contig3.1b, whole
genome shotgun sequence; 926412094; NZ_LGDY01000103.1
3663; Nocardia sp. NRRL S-836 P437contig39.1, whole
genome shotgun sequence; 926412104; NZ_LGDY01000113.1
3664; Paenibacillus sp. A59 contig_353, whole genome
shotgun sequence; 927084730; NZ_LITU01000050.1
3665; Paenibacillus sp. A59 contig_416, whole genome
shotgun sequence; 927084736; NZ_LITU01000056.1
3666; Streptomyces sp. XY332 P409contig34.1, whole genome
shotgun sequence; 927093145; NZ_LGHN01000166.1
3667; Streptomyces rimosus subsp. rimosus strain NRRL
WC-3898 P259contig86.1, whole genome shotgun
sequence; 927279089; NZ_LGCU01000353.1
3668; Streptomyces rimosus subsp. pseudoverticillatus strain
NRRL WC-3896 P270contig51.1, whole genome shotgun
sequence; 927292651; NZ_LGCV01000382.1
3669; Streptomyces rimosus subsp. pseudoverticillatus strain
NRRL WC-3896 P270contig8.1, whole genome shotgun
sequence; 927292684; NZ_LGCV01000415.1
3670; Aneurinibacillus migulanus strain Nagano E1 contig_36,
whole genome shotgun sequence; 928874573; NZ_LIXL01000208.1
3671; Streptomyces chattanoogensis strain NRRL ISP-5002
ISP5002contig8.1, whole genome shotgun sequence; 928897585;
NZ_LGKG01000196.1
3672; Streptomyces chattanoogensis strain NRRL ISP-5002
ISP5002contig9.1, whole genome shotgun sequence; 928897596;
NZ_LGKG01000207.1
3673; Streptomyces sp. NRRL F-6602 F6602contig54.1, whole
genome shotgun sequence; 928910033; NZ_LGKH01004848.1
3674; Ideonella sakaiensis strain 201-F6, whole genome
shotgun sequence; 928998724; NZ_BBYR01000007.1
3675; Ideonella sakaiensis strain 201-F6, whole genome
shotgun sequence; 928998800; NZ_BBYR01000083.1
3676; Bacillus sp. FJAT-28004 scaffold_2, whole genome
shotgun sequence; 929005248; NZ_LGHP01000003.1
3677; Novosphingobium sp. AAP1 AAP1Contigs7, whole
genome shotgun sequence; 930029075; NZ_UHO01000007.1
3678; Novosphingobium sp. AAP1 AAP1Contigs9, whole
genome shotgun sequence; 930029077; NZ_LJHO01000009.1
3679; Stenotrophomonas maltophilia strain OC194 contig_98,
whole genome shotgun sequence; 930169273; NZ_LEH01000098.1
3680; Actinobacteria bacterium OK074 ctg60, whole genome
shotgun sequence; 930473294; NZ_LJCV01000275.1
3681; Actinobacteria bacterium OK006 ctg112, whole genome
shotgun sequence; 930490730; NZ_LJCU01000014.1
3682; Actinobacteria bacterium OK006 ctg96, whole genome
shotgun sequence; 930491003; NZ_LJCU01000287.1
3683; Kibdelosporangium phytohabitans strain KLBMP1111,
complete genome; 931609467; NZ_CP012752.1
3684; Streptococcus pneumoniae strain P18082 isolate E3GXY,
whole genome shotgun sequence; 935445269; NZ_CIEC02000098.1
3685; Paenibacillus solani strain FJAT-22460 super3, whole
genome shotgun sequence; 935460965; NZ_LIUT01000006.1
3686; Novosphingobium sp. ST904 contig_104, whole genome
shotgun sequence 935540718; NZ_LGJH01000063.1
3687; Citromicrobium sp. RCC1878 contig2, whole genome
shotgun sequence; 936191447; NZ_LBLZ01000002.1
3688; Frankia sp. R43 contig001, whole genome shotgun
sequence; 937182893; NZ_LFCW01000001.1
3689; Sphingopyxis macrogoltabida strain EY-1, complete
genome; 937372567; NZ_CP012700.1
3690; Sphingopyxis macrogoltabida strain EY-1, complete
genome; 937372567; NZ_CP012700.1
3691; Xanthomonas arboricola strain CITA 44 CITA_44_contig 26,
whole genome shotgun sequence; 937505789; NZ_LJGM01000026.1
3692; Stenotrophomonas acidaminiphila strain ZAC14D2_
NAIMI4_2, complete genome; 938883590; NZ_CP012900.1
3693; Sphingopyxis macrogoltabida strain 203, complete
genome; 938956730; NZ_CP009429.1
3694; Sphingopyxis macrogoltabida strain 203, complete
genome; 938956730; NZ_CP009429.1
3695; Sphingopyxis macrogoltabida strain 203 plasmid,
complete sequence; 938956814; NZ_CP009430.1
3696; Cellulosilyticum ruminicola JCM 14822, whole genome
shotgun sequence; 938965628; NZ_BBCG01000065.1
3697; Brevundimonas sp. DS20, complete genome; 938989745;
NZ_CP012897.1
3698; Brevundimonas sp. D520, complete genome; 938989745;
NZ_CP012897.1
3699; Paenibacillus sp. GD6, whole genome shotgun sequence;
939708098; NZ_LN831198.1
3700; Paenibacillus sp. GD6, whole genome shotgun sequence;
939708105; NZ_LN831205.1
3701; Alicyclobacillus ferrooxydans strain TC-34 contig 22, whole
genome shotgun sequence; 940346731; NZ_LJC001000107.1
3702; Xanthomonas sp. Mitacek01 contig_17, whole genome
shotgun sequence; 941965142; NZ_LKIT01000002.1
3703; Streptomyces bingchenggensis BCW-1, complete
genome; 374982757; NC_016582.1
3704; Streptomyces pactum strain ACT12 scaffold1, whole
genome shotgun sequence; 943388237; NZ_LIQD01000001.1
3705; Streptomyces flocculus strain NRRL B-2465 B2465_
contig_205, whole genome shotgun sequence; 943674269;
NZ_LIQ001000205.1
3706; Streptomyces aurantiacus strain NRRL ISP-5412 ISP-5412_
contig_138, whole genome shotgun sequence; 943881150;
NZ_LIPP01000138.1
3707; Streptomyces graminilatus strain NRRL B-59124 B59124_
contig_7, whole genome shotgun sequence; 943897669;
NZ_LIQQ01000007.1
3708; Streptomyces alboniger strain NRRL B-1832 B-1832_
contig_37, whole genome shotgun sequence; 943898694;
NZ_LIQN01000037.1
3709; Streptomyces alboniger strain NRRL B-1832 B-1832_
contig_384, whole genome shotgun sequence; 943899498;
NZ_LIQN01000384.1
3710; Streptomyces kanamyceticus strain NRRL B-2535 B-2535_
contig_122, whole genome shotgun sequence; 943922224;
NZ_LIQU01000122.1
3711; Streptomyces kanamyceticus strain NRRL B-2535 B-2535_
contig_247, whole genome shotgun sequence; 943922567;
NZ_LIQU01000247.1
3712; Streptomyces luridiscabiei strain NRRL B-24455 B24455_
contig_315, whole genome shotgun sequence; 943927948;
NZ_LIQV01000315.1
3713; Streptomyces attiruber strain NRRL B-24165 contig_
124, whole genome shotgun sequence; 943949281;
NZ_LIPN01000124.1
3714; Streptomyces hirsutus strain NRRL B-2713 B2713_
contig_57, whole genome shotgun sequence; 944005810;
NZ_LIQT01000057.1
3715; Streptomyces aureus strain NRRL B-2808 contig 171, whole
genome shotgun sequence; 944012845; NZ_LIPQ01000171.1
3716; Streptomyces prasinus strain NRRL B-12521 B12521_
contig_230, whole genome shotgun sequence; 944020089;
NZ_LIPR01000230.1
3717; Streptomyces phaeochromogenes strain NRRL B-1248 B-
1248_contig_126, whole genome shotgun sequence; 944029528;
NZ_LIQZ01000126.1
3718; Streptomyces prasinus strain NRRL B-2712 B2712_
contig_323, whole genome shotgun sequence; 944410649;
NZ_LIRH01000323.1
3719; Streptomyces prasinopilosus strain NRRL B-2711
B2711_contig_370, whole genome shotgun sequence; 944415035;
NZ_LIRG01000370.1
3720; Streptomyces torulosus strain NRRL B-3889 B-3889_
contig_18, whole genome shotgun sequence; 944495433;
NZ_LIRK01000018.1
3721; Frankia alni str. ACN14A chromosome, complete
sequence; 111219505; NC_008278.1
3722; Frankia sp. CpI1-S FF36_scaffold_9.10, whole genome
shotgun sequence; 768715243; NZ_JYFN01000010.1
3723; Sphingomonas sp. Leaf20 contig_1, whole genome
shotgun sequence; 947349881; NZ_LMKN01000001.1
3724; Paenibacillus sp. Leaf72 contig_6, whole genome
shotgun sequence; 947378267; NZ_LMLV01000032.1
3725; Sphingomonas sp. Leaf230 contig 4, whole genome
shotgun sequence; 947401208; NZ_LMKW01000010.1
3726; Sanguibacter sp. Leaf3 contig_2, whole genome
shotgun sequence; 947472882; NZ_LMRH01000002.1
3727; Aeromicrobium sp. Root344 contig_1, whole genome
shotgun sequence; 947552260; NZ_LMDH01000001.1
3728; Sphingopyxis sp. Root1497 contig_3, whole genome
shotgun sequence; 947689975; NZ_LMGF01000003.1
3729; Sphingomonas sp. Root1294 contig_7, whole genome
shotgun sequence; 947890193; NZ_LMEJ01000014.1
3730; Sphingomonas sp. Root720 contig_7, whole genome
shotgun sequence; 947704642; NZ_LMID01000015.1
3731; Sphingomonas sp. Root720 contig_8, whole genome
shotgun sequence; 947704650; NZ_LMID01000016.1
3732; Sphingomonas sp. Root710 contig_1, whole genome
shotgun sequence; 947721816; NZ_LMLB01000001.1
3733; Sphingomonas sp. Root1294 contig_7, whole genome
shotgun sequence; 947890193; NZ_LMEJ01000014.1
3734; Mesorhizobium sp. Root172 contig_2, whole genome
shotgun sequence; 947919015; NZ_LMHP01000012.1
3735; Mesorhizobium sp. Root102 contig_3, whole genome
shotgun sequence; 947937119; NZ_LMCP01000023.1
3736; Paenibacillus sp. 5oi1750 contig_1, whole genome
shotgun sequence; 947966412; NZ_LMSD01000001.1
3737; Paenibacillus sp. 5oi1522 contig_3, whole genome
shotgun sequence; 947983982; NZ_LMRV01000044.1
3738; Paenibacillus sp. 5oi1522 contig_3, whole genome
shotgun sequence; 947983982; NZ_LMRV01000044.1
3739; Paenibacillus sp. Root52 contig_3, whole genome
shotgun sequence; 948045460; NZ_LMFO01000023.1
3740; Enterococcus faecalis ATCC 29212 contig24, whole
genome shotgun sequence; 401673929; ALOD01000024.1
3741; Mesorhizobium sp. Root695 contig_1, whole genome
shotgun sequence; 950019035; NZ_LMH001000001.1
3742; Bacillus sp. Soil768D1 contig_5, whole genome
shotgun sequence; 950170460; NZ_LMTA01000046.1
3743; Paenibacillus sp. Root444D2 contig_4, whole genome
shotgun sequence; 950271971; NZ_LME001000034.1
3744; Paenibacillus sp. Soil766 contig_32, whole genome
shotgun sequence; 950280827; NZ_LMSJ01000026.1
3745; Streptococcus pneumoniae strain type strain: N, whole
genome shotgun sequence; 950938054; NZ_CIHL01000007.1
3746; Streptomyces sp. Root1310 contig_5, whole genome
shotgun sequence; 951121600; NZ_LMEQ01000031.1
3747; Bacillus mumlis strain DSM 16288 Scaffold4, whole
genome shotgun sequence; 951610263; NZ_LMBV01000004.1
3748; Streptomyces sp. MBT76 scaffold_2, whole genome
shotgun sequence; 953813788; NZ_LNBE01000002.1
3749; Streptomyces sp. MBT76 scaffold_3, whole genome
shotgun sequence; 953813789; NZ_LNBE01000003.1
3750; Streptomyces sp. MBT76 scaffold_4, whole genome
shotgun sequence; 953813790; NZ_LNBE01000004.1
3751; Clostridium butyricum strain KN1J-L09 chromosome
1, complete sequence; 959868240; NZ_CP013252.1
3752; Clostridium butyricum strain NEC8, whole genome
shotgun sequence; 960334134; NZ_CBYK010000003.1
3753; Gorillibacterium sp. SN4, whole genome shotgun
sequence; 960412751; NZ_LN881722.1
3754; Thalassobius activus strain CECT 5114, whole genome
shotgun sequence; 960424655; NZ_CYUE01000025.1
3755; Microbacterium testaceum strain N5283 contig_37, whole
genome shotgun sequence; 969836538; NZ_LDRU01000037.1
3756; Microbacterium testaceum strain N5206 contig_27, whole
genome shotgun sequence; 969912012; NZ_LDRS01000027.1
3757; Microbacterium testaceum strain N5183 contig_65, whole
genome shotgun sequence; 969919061; NZ_LDRR01000065.1
3758; Paenibacillus jamilae strain NS115 contig_7, whole
genome shotgun sequence; 970428876; NZ_LDRX01000027.1
3759; Sphingopyxis sp. H050 H050_contig000006, whole
genome shotgun sequence; 970555001; NZ_LNRZ01000006.1
3760; Paenibacillus polymyxa strain KF-1 scaffold00001, whole
genome shotgun sequence; 970574347; NZ_LNZF01000001.1
3761; Luteimonas abyssi strain XH031 Scaffold1, whole
genome shotgun sequence; 970579907; NZ_KQ759763.1

TABLE 5
Exemplary Lasso RRE
Lasso RRE Peptide No: #; Species of Origin; GI #; Accession #

3762; Geobacter uraniireducens Rf4, complete genome; 148262085;
NC_009483.1
3763; Sphingomonas wittichii RW1, complete genome; 148552929;
NC_009511.1
3764; Sanguibacter keddieii DSM 10542, complete genome; 269793358;
NC_013521.1
3765; Xylanimonas cellulosilytica DSM 15894, complete genome; 269954810;
NC_013530.1
3766; Spirosoma linguale DSM 74, complete genome; 283814236; CP001769.1
3767; Streptomyces bingchenggensis BCW-1, complete genome; 374982757;
NC_016582.1
3768; Streptomyces bingchenggensis BCW-1, complete genome; 374982757;
NC_016582.1
3769; Gallionella capsifeniformans ES-2, complete genome; 302877245;
NC_014394.1
3770; Mycobacterium sinense strain JDM601, complete genome; 333988640;
NC_015576.1
3771; Streptomyces violaceusniger Tu 4113, complete genome; 345007964;
NC_015957.1
3772; Rhodospirillum rubrum F11, complete genome; 386348020; NC_017584.1
3773; Actinoplanes sp. SE50/110, complete genome; 386845069; NC_017803.1
3774; Emticicia oligotrophica DSM 17448, complete genome; 408671769;
NC_018748.1
3775; Tistrellamobilis KA081020-065 plasmid pTM1, complete sequence;
442559580; NC_017957.2
3776; Bacillus thuringiensis MC28, complete genome; 407703236; NC_018693.1
3777; Nostoc sp. PCC 7107, complete genome; 427705465; NC_019676.1
3778; Synechococcus sp. PCC 6312, complete genome; 427711179;
NC_019680.1
3779; Stanieria cyanosphaera PCC 7437, complete genome; 428267688;
CP003653.1
3780; Desulfocapsa sulfexigens DSM 10523, complete genome; 451945650;
NC_020304.1
3781; Xanthomonas citri pv. punicae str. LMG 859, whole genome shotgun
sequence; 390991205; NZ_CAGJ01000031.1
3782; Streptomyces fulvissimus DSM 40593, complete genome; 488607535;
NC_021177.1
3783; Streptomyces rapamycinicus NRRL 5491 genome; 521353217;
CP006567.1
3784; Gloeobacter kilaueensis JS1, complete genome; 554634310; NC_022600.1
3785; Gloeobacter kilaueensis JS1, complete genome; 554634310; NC_022600.1
3786; Kutzneria albida strain NRRL B-24060 contig305.1, whole genome shotgun
sequence; 662161093; NZ_JNYH01000515.1
3787; Kutzneria albida strain NRRL B-24060 contig305.1, whole genome shotgun
sequence; 662161093; NZ_JNYH01000515.1
3788; Mesorhizobium huakuii 7653R genome; 657121522; CP006581.1
3789; Sphingopyxis fiibergensis strain Kp5.2, complete genome; 749188513;
NZ_CP009122.1
3790; Amycolatopsis lurida NRRL 2430, complete genome; 755908329;
CP007219.1
3791; Streptomyces lydicus A02, complete genome; 822214995;
NZ_CP007699.1
3792; Streptomyces lydicus A02, complete genome; 822214995;
NZ_CP007699.1
3793; Uncultured bacterium clone AZ25P121 genomic sequence; 818476494;
KP274854.1
3794; Streptomyces sp. PBH53 genome; 852460626; CP011799.1
3795; Streptomyces sp. PBH53 genome; 852460626; CP011799.1
3796; Streptomyces sp. PBH53 genome; 852460626; CP011799.1
3797; Bifidobacterium longum subsp infantis strain BT1, complete genome;
927296881; CP010411.1
3798; Nostoc piscinale CENA21 genome; 930349143; CP012036.1
3799; Paenibacillus sp. 320-W, complete genome; 961447255; CP013653.1
3800; Streptomyces avermitilis MA-4680 = NBRC 14893, complete genome;
162960844; NC_003155.4
3801; Streptomyces avermitilis MA-4680 = NBRC 14893, complete genome;
162960844; NC_003155.4
3802; Kitasatospora setae KM-6054 DNA, complete genome; 357386972;
NC_016109.1
3803; Rhodococcus jostii lariatin biosynthetic gene cluster (larA, larB, larC, larD, larE),
complete cds; 380356103; AB593691.1
3804; Pseudomonas sp. St29 DNA, complete genome; 771846103; AP014628.1
3805; Pseudomonas sp. St29 DNA, complete genome; 771846103; AP014628.1
3806; Fischerella sp. NIES-3754 DNA, complete genome; 965684975;
AP017305.1
3807; Magnetospirillum gryphiswaldense MSR-1, WORKING DRAFT
SEQUENCE, 373 unordered pieces; 144897097; CU459003.1
3808; Streptococcus suis 98HAH33, complete genome; 145690656; CP000408.1
3809; Salinibacter ruber M8 chromosome, complete genome; 294505815;
NC_014032.1
3810; Enterococcus faecalis strain P9-1 scaffold484.1, whole genome shotgun
sequence; 949763393; NZ_LKGS01000484.1
3811; Saccharothrix espanaensis DSM 44229 complete genome; 433601838;
NC_019673.1
3812; Roseburia sp. CAG:197 WGS project CBBL01000000 data, contig, whole
genome shotgun sequence; 524261006; CBBL010000225.1
3813; Clostridium sp. CAG:221 WGS project CBDC01000000 data, contig,
whole genome shotgun sequence; 524362382; CBDC010000065.1
3814; Clostridium sp. CAG:411 WGS project CBIY01000000
genome shotgun sequence; 524742306; CBIY010000075.1
3815; Roseburia sp. CAG:100 WGS project CBKV01000000 data, contig, whole
genome shotgun sequence; 524842500; CBKV010000277.1
3816; Mesorhizobium plurifarium, whole genome shotgun sequence; 751292755;
NZ_CCNE01000004.1
3817; Kibdelosporangium sp. MJ126-NF4, whole genome shotgun sequence;
754819815; NZ_CDME01000002.1
3818; Kibdelosporangium sp. MJ126-NF4 genome assembly High
quaKibdelosporangium sp. MJ126-NF4, scaffold BPA_8, whole genome shotgun
sequence; 747653426; CDME01000011.1
3819; Methanobacterium formicicum genome assembly isolate Mb9,
chromosome : I; 952971377; LN734822.1
3820; Streptococcus pneumoniae strain type strain: N, whole genome shotgun
sequence; 950938054; NZ_CIHL01000007.1
3821; Streptococcus pneumoniae strain type strain: N, whole genome shotgun
sequence; 950938054; NZ_CIHL01000007.1
3822; Streptococcus pneumoniae strain type strain: N, whole genome shotgun
sequence; 950938054; NZ_CIHL01000007.1
3823; Bacillus cereus genome assembly Bacillus JRS4, contig contig000025,
whole genome shotgun sequence; 924092470; CYHM01000025.1
3824; Pedobacter sp. BAL39 1103467000492, whole genome shotgun sequence;
149277373; NZ_ABCM01000005.1
3825; Streptomyces sviceus ATCC 29083 chromosome, whole genome shotgun
sequence; 297196766; NZ_CM000951.1
3826; Streptomyces pristinaespiralis ATCC 25486 chromosome, whole genome
shotgun sequence; 297189896; NZ_CM000950.1
3827; Enterococcus faecalis strain P9-1 scaffold484.1, whole genome shotgun
sequence; 949763393; NZ_LKGS01000484.1
3828; Enterococcus faecalis strain P9-1 scaffold484.1, whole genome shotgun
sequence; 949763393; NZ_LKGS01000484.1
3829; Streptomyces sp. CN5654 CD02DRAFT_scaffold00023.23_C, whole
genome shotgun sequence; 695856316; NZ_INLT01000024.1
3830; Streptococcus vestibularis F0396 ctg1126932565723, whole genome
shotgun sequence; 311100538; AEK001000007.1
3831; Ruminococcus albus 8 contig00035, whole genome shotgun sequence;
data, contig, whole 325680876; NZ_ADKM02000123.1
3832; Streptomyces sp. W007 contig00293, whole genome shotgun sequence;
365867746; NZ_AGSW01000272.1
3833; Streptomyces auratus AGR0001 Scaffold1_85, whole genome shotgun
sequence; 396995461; AJGV01000085.1
3834; Actinomyces naeslundii str. Howell 279 ctg1130888818142, whole genome
shotgun sequence; 399903251; ALJK01000024.1
3835; Enterococcus faecalis strain P9-1 scaffold484.1, whole genome shotgun
sequence; 949763393; NZ_LKGS01000484.1
3836; Amycolatopsis decaplanina DSM 44594 Contig0055, whole genome
shotgun sequence; 458848256; NZ_AOHO01000055.1
3837; Streptomyces mobaraensis NBRC 13819 = DSM 40847 contig024, whole
genome shotgun sequence; 458977979; NZ_AORZ01000024.1
3838; Enterococcus faecalis strain P9-1 scaffold484.1, whole genome shotgun
sequence; 949763393; NZ_LKGS01000484.1
3839; Enterococcus faecalis strain P9-1 scaffold484.1, whole genome shotgun
sequence; 949763393; NZ_LKGS01000484.1
3840; Streptomyces aurantiacus JA 4570 Seq28, whole genome shotgun sequence;
514916412; NZ_AOPZ01000028.1
3841; Streptomyces aurantiacus JA 4570 Seq17, whole genome shotgun sequence;
514916021; NZ_AOPZ01000017.1
3842; Enterococcus faecalis strain P9-1 scaffold484.1, whole genome shotgun
sequence; 949763393; NZ_LKGS01000484.1
3843; Paenibacillus alvei A6-6i-x PAAL66ix_14, whole genome shotgun
sequence; 528200987; ATMS01000061.1
3844; Dehalobacter sp. UNSWDHB Contig_139, whole genome shotgun
sequence; 544905305; NZ_AUUR01000139.1
3845; Actinobaculum sp. oral taxon 183 str. F0552 A_P1HMPREF0043-
1.0 Cont15.2, whole genome shotgun sequence; 541473965; AWSB01000041.1
3846; Actinobaculum sp. oral taxon 183 str. F0552 A_P1HMPREF0043-
1.0_Cont1.1, whole genome shotgun sequence; 541476958; AWSB01000006.1
3847; Propionibacterium acidifaciens F0233 ctg1127964738299, whole genome
shotgun sequence; 544249812; ACVN02000045.1
3848; Rubidibacter lacunae KORDI 51-2 KR5 l_contig00121, whole genome
shotgun sequence; 550281965; NZ_ASSJ01000070.1
3849; Rothia aeria F0184 R aerigIMPREF0742-1.0_Cont136.4, whole genome
shotgun sequence; 551695014; AXZGO1000035.1
3850; Candidatus Halobonum tyrrellensis G22 contig00002, whole genome
shotgun sequence; 557371823; NZ_ASGZ01000002.1
3851; Streptomyces niveus NCIMB 11891 contig00003, whole genome shotgun
sequence; 558542923; AWQW01000003.1
3852; Frankia sp. Thr ThrDRAFT_scaffold_28.29, whole genome shotgun
sequence; 602262270; JENI01000029.1
3853; Clostridium butyricum DORA_1 Q607_CBUC00058, whole genome
shotgun sequence; 566226100; AZLX01000058.1
3854; Streptococcus sp. DORA_10 Q617_5P5C00257, whole genome shotgun
sequence; 566231608; AZMH01000257.1
3855; Candidatus Entotheonella factor TSYl_contig00913, whole genome
shotgun sequence; 575408569; AZHW01000959.1
3856; Streptomyces sp. CN5654 CD02DRAFT_scaffold00023.23S, whole
genome shotgun sequence; 695856316; NZ_JNLT01000024.1
3857; Frankia sp. Thr ThrDRAFT_scaffold_28.29, whole genome shotgun
sequence; 602262270; JENI01000029.1
3858; Bacillus akibai JCM 9157, whole genome shotgun sequence; 737696658;
NZ_BAUV01000025.1
3859; Bacillus boroniphilus JCM 21738 DNA, contig: contig 6, whole genome
shotgun sequence; 571146044; BAUW01000006.1
3860; Gracilibacillus boraciitolerans JCM 21714 DNA, contig:contig_30, whole
genome shotgun sequence; 575082509; BAVS01000030.1
3861; Streptomyces griseorubens strain JSD-1 contig143, whole genome shotgun
sequence; 657284919; BMG01000143.1
3862; Frankia sp. CeD CEDDRAFT_scaffold_22.23, whole genome shotgun
sequence; 737947180; NZ_JPGU01000023.1
3863; Frankia sp. Thr ThrDRAFT_scaffold 28.29, whole genome shotgun
sequence; 602262270; JENI01000029.1
3864; Streptomyces sp. JS01 contig2, whole genome shotgun sequence;
695871554; NZ_JPWW01000002.1
3865; Rothia dentocariosa strain C6B contig 5, whole genome shotgun sequence;
739372122; NZ_JQHE01000003.1
3866; Candidatus Thiomargarita nelsonii isolate Hydrate Ridge contig_1164,
whole genome shotgun sequence; 723288710; JSZA01001164.1
3867; Streptomyces globisporus C-1027 Scaffold24_1, whole genome shotgun
sequence; 410651191; NZ_AJU001000171.1
3868; Lechevalieria aerocolonigenes strain NRRL B-16140 contig11.3, whole
genome shotgun sequence; 772744565; NZ_JYJG01000059.1
3869; Desulfobulbaceae bacterium BRH_c16a BRHa_1001515, whole genome
shotgun sequence; 780791108; LADS01000058.1
3870; Peptococcaceae bacterium BRH_c4b BRHa_1001357, whole genome
shotgun sequence; 780813318; LAD001000010.1
3871; Streptomyces rubellomurinus subsp. indigoferus strain ATCC 31304 contig-55,
whole genome shotgun sequence; 783374270; NZ_JZKG01000056.1
3872; Streptomyces sp. NRRL S-444 contig322.4, whole genome shotgun
sequence; 797049078; JZWX01001028.1
3873; Streptomyces sp. NRRL B-1568 contig-76, whole genome shotgun
sequence; 799161588; NZ_JZWZ01000076.1
3874; Streptomyces caatingaensis strain CMAA 1322 contig02, whole genome
shotgun sequence; 906344334; NZ_LFXA01000002.1
3875; Paenibacillus polymyxa strain YUPP-8 scaffold32, whole genome shotgun
sequence; 924434005; LIYK01000027.1
3876; Streptomyces rimosus subsp. rimosus strain NRRL B-16073 contig48.1,
whole genome shotgun sequence; 696497741; NZ_JNWX01000048.1
3877; Streptomyces rimosus subsp. rimosus ATCC 10970 contig00333, whole
genome shotgun sequence; 441178796; NZ_ANSJ01000259.1
3878; Streptomyces rimosus subsp. rimosus strain NRRL B-16073 contig48.1,
whole genome shotgun sequence; 696497741; NZ_JNWX01000048.1
3879; Streptomyces rimosus subsp. rimosus strain NRRL WC-3869
P248contig20.1, whole genome shotgun sequence; 925322461;
LGCQ01000113.1
3880; Streptomyces rimosus subsp. rimosus ATCC 10970 contig00333, whole
genome shotgun sequence; 441178796; NZ_ANSJ01000259.1
3881; Streptomyces rimosus subsp. rimosus ATCC 10970 contig00333, whole
genome shotgun sequence; 441178796; NZ_ANSJ01000259.1
3882; Streptomyces rimosus subsp. rimosus strain NRRL B-16073 contig48.1,
whole genome shotgun sequence; 696497741; NZ_JNWX01000048.1
3883; Streptomyces rimosus subsp. rimosus strain NRRL B-2660 contig124.1,
whole genome shotgun sequence; 664066234; NZ_JOES01000124.1
3884; Streptomyces sp. NRRL F-5755 P309contig50.1, whole genome shotgun
sequence; 926371520; NZ_LGCW01000274.1
3885; Streptomyces sp. NRRL F-5755 P309contig48.1, whole genome shotgun
sequence; 926371517; NZ_LGCW01000271.1
3886; Streptomyces sp. NRRL S-444 contig322.4, whole genome shotgun
sequence; 797049078; JZWX01001028.1
3887; Actinobacteria bacterium 01006 ctg96, whole genome shotgun sequence;
930491003; NZ_LJCU01000287.1
3888; Actinobacteria bacterium OK074 ctg60, whole genome shotgun sequence;
930473294; NZ_LJCV01000275.1
3889; Betaproteobacteria bacterium 5G8 39 WOR 8-12 2589, whole genome
shotgun sequence; 931421682; LJTQ01000030.1
3890; Candidate division BRC1 bacterium SM23_51 WORSMTZ_10094, whole
genome shotgun sequence; 931536013; LJUL01000022.1
3891; Bacillus vietnamensis strain UCD-SED5 scaffold_15, whole genome
shotgun sequence; 933903534; LIXZ01000017.1
3892; Erythrobacteraceae bacterium HL-111 ITZY_scaf_51, whole genome
shotgun sequence; 938259025; LJSW01000006.1
3893; Halomonas sp. HL-93 ITZY_scaf 415, whole genome shotgun sequence;
938285459; LJST01000237.1
3894; Paenibacillus sp. Soi1724D2 contig_11, whole genome shotgun sequence;
946400391; LMRY01000003.1
3895; Paenibacillus sp. Root444D2 contig_4, whole genome shotgun sequence;
950271971; NZ_LME001000034.1
3896; Streptomyces silvensis strain ATCC 53525 53525 Assembly_Contig_22,
whole genome shotgun sequence; 970361514; LOCL01000028.1
3897; Bacillus mycoides strain Flugge 10206 DJ94.contig-100_16, whole genome
shotgun sequence; 727343482; NZ_JMQD01000030.1
3898; Bacillus cereus BAG3X2-1 supercont1.1, whole genome shotgun sequence;
423416528; NZ_JH791923.1
3899; Bacillus thuringiensis MC28, complete genome; 407703236; NC_018693.1
3900; Bacillus cereus VD131 acrHi-supercont1.9, whole genome shotgun
sequence; 507037581; NZ_KB976660.1
3901; Bacillus cereus Rock4-18 chromosome, whole genome shotgun sequence;
238801487; NZ_CM000735.1
3902; Bacillus cereus AH1271 chromosome, whole genome shotgun sequence;
238801491; NZ_CM000739.1
3903; Bacillus cereus Rock3-44 chromosome, whole genome shotgun sequence;
238801485; NZ_CM000733.1
3904; Bacillus cereus VD115 supercont1.1, whole genome shotgun sequence;
423614674; NZ_JH792165.1
3905; Bacillus sp. UMTAT18 contig000011, whole genome shotgun sequence;
806951735; NZ_JSFD01000011.1
3906; Bacillus cereus BAG5X2-1 supercont1.1, whole genome shotgun sequence;
423456860; NZ_JH791975.1
3907; Streptococcus pneumoniae strain type strain: N, whole genome shotgun
sequence; 950938054; NZ_CIHL01000007.1
3908; Bacillus cereus VD142 actaa-supercont2.2, whole genome shotgun
sequence; 514340871; NZ_KE150045.1
3909; Bacillus cereus BAG60-2 supercont1.1, whole genome shotgun sequence;
423468694; NZ_JH804628.1
3910; Bacillus mycoides FSL H7-687 Contig052, whole genome shotgun
sequence; 727271768; NZ_ASPY01000052.1
3911; Bacillus cereus HuA2-9 acqVt-supercont1.1, whole genome shotgun
sequence; 507020427; NZ_KB976152.1
3912; Bacillus cereus HuA4-10 supercont1.1, whole genome shotgun sequence;
423520617; NZ_JH792148.1
3913; Bacillus cereus MC67 supercont1.2, whole genome shotgun sequence;
423557538; NZ_JH792114.1
3914; Bacillus cereus AH621 chromosome, whole genome shotgun sequence;
238801471; NZ_CM000719.1
3915; Bacillus cereus VD107 supercont1.1, whole genome shotgun sequence;
423609285; NZ_JH792232.1
3916; Bacillus cereus VDM034 supercont1.1, whole genome shotgun sequence;
423666303; NZ_JH791809.1
3917; Bacillus cereus BAG5X1-1 supercont1.1, whole genome shotgun sequence;
423451256 ;NZ_JH791996.1
3918; Enterococcus faecalis strain P9-1 scaffold484.1, whole genome shotgun
sequence; 949763393; NZ_LKGS01000484.1
3919; Clostridium butyricum 5521 gcontig_1106103650482, whole genome
shotgun sequence; 182420360; NZ_ABDT01000120.2
3920; Rhodobacter sphaeroides WS8N chromosome chrI, whole genome shotgun
sequence; 332561612; NZ_CM001161.1
3921; Methylosinus ttichosporium OB3b MettrDRAFT_Contig106_C, whole
genome shotgun sequence; 639846426; NZ_ADVE02000001.1
3922; Streptomyces clavuligerus ATCC 27064 supercont1.55, whole genome
shotgun sequence; 254392242; NZ_DS570678.1
3923; Streptomyces rimosus subsp. rimosus strain NRRL B-16073 contig48.1,
whole genome shotgun sequence; 696497741; NZ_JNWX01000048.1
3924; Streptomyces rimosus subsp. rimosus ATCC 10970 contig00333, whole
genome shotgun sequence; 441178796; NZ_ANSJ01000259.1
3925; Streptomyces viridochromogenes DSM 40736 supercont1.1, whole genome
shotgun sequence; 224581107; NZ_GG657757.1
3926; Streptomyces viridochromogenes DSM 40736 supercont1.1, whole genome
shotgun sequence; 224581107; NZ_GG657757.1
3927; Methanobacterium formicicum DSM 3637 Contig04, whole genome
shotgun sequence; 408381849; NZ_AMP001000004.1
3928; Methanobacterium formicicum DSM 3637 Contig04, whole genome
shotgun sequence; 408381849; NZ_AMP001000004.1
3929; Sphingobium yanoikuyae strain SHJ scaffold2, whole genome shotgun
sequence; 893711333; NZ_KQ235984.1
3930; Streptomyces mobaraensis NBRC 13819 = DSM 40847 contig024, whole
genome shotgun sequence; 458977979; NZ_AORZ01000024.1
3931; Streptomyces mobaraensis NBRC 13819 = DSM 40847 contig079, whole
genome shotgun sequence; 458984960; NZ_AORZ01000079.1
3932; Amycolatopsis azurea DSM 43854 contig60, whole genome shotgun
sequence; 451338568; NZ_ANMG01000060.1
3933; Streptomyces pristinaespiralis ATCC 25486 chromosome, whole genome
shotgun sequence; 297189896; NZ_CM000950.1
3934; Xanthomonas citri pv. punicae str. LMG 859, whole genome shotgun
sequence; 390991205; NZ_CAGJO1000031.1
3935; Streptomyces sp. CNS654 CD02DRAFT_scaffold00023.23_C, whole
genome shotgun sequence; 695856316; NZ_JNLT01000024.1
3936; Mesorhizobium amolphae CCNWGS0123 contig00204, whole genome
shotgun sequence; 357028583; NZ_AGSNO1000187.1
3937; Leptolyngbya sp. PCC 7375 Lepto7375DRAFT_LPA.5, whole genome
shotgun sequence; 427415532; NZ_JH993797.1
3938; Streptomyces auratus AGR0001 Scaffold1, whole genome shotgun
sequence; 398790069; NZ_JH725387.1
3939; Streptomyces auratus AGR0001 Scaffold1_85, whole genome shotgun
sequence; 396995461; AJGV01000085.1
3940; Paenibacillus dendritiformis C454 PDENDC1000064, whole genome
shotgun sequence; 374605177; NZ_AHKH01000064.1
3941; Halosimplex carlsbadense 2-9-1 contig 4, whole genome shotgun sequence;
448406329; NZ_AOIU01000004.1
3942; Amycolatopsis decaplanina DSM 44594 Contig0055, whole genome
shotgun sequence; 458848256; NZ_AOH001000055.1
3943; Fictibacillus macauensis ZFHKF-1 Contig20, whole genome shotgun
sequence; 392955666; NZ_AKKV01000020.1
3944; Streptomyces sviceus ATCC 29083 chromosome, whole genome shotgun
sequence; 297196766; NZ_CM000951.1
3945; Paenibacillus sp. Aloe-11 GW8_15, whole genome shotgun sequence;
375307420; NZ_JH601049.1
3946; Streptomyces sp. W007 contig00293, whole genome shotgun sequence;
365867746; NZ_AGSW01000272.1
3947; Frankia saprophytica strain CN3 FrCN3DRAFT FCB.2, whole genome
shotgun sequence; 652876473; NZ_KI912267.1
3948; Desulfosporosinus youngiae DSM 17734 chromosome, whole genome
shotgun sequence; 374578721; NZ_CM001441.1
3949; Mooreaproducens 3L scf52054, whole genome shotgun sequence;
332710503; NZ_GL890955.1
3950; Pedobacter sp. BAL39 1103467000500, whole genome shotgun sequence;
149277003; NZ_ABCM01000004.1
3951; Pedobacter sp. BAL39 1103467000492, whole genome shotgun sequence;
149277373; NZ_ABCM01000005.1
3952; Sulfurovum sp. AR contig00449, whole genome shotgun sequence;
386284588; NZ_AJLE01000006.1
3953; Mucilaginibacter paludis DSM 18603 chromosome, whole genome shotgun
sequence; 373951708; NZ_CM001403.1
3954; Magnetospinllum caucaseum strain SO-1 contig00006, whole genome
shotgun sequence; 458904467; NZ_AONQ01000006.1
3955; Mooreaproducens 3L scf52052, whole genome shotgun sequence;
332710285; NZ_GL890953.1
3956; Cecembia lonarensis LW9 contig000133, whole genome shotgun sequence;
406663945; NZ_AMGM01000133.1
3957; Actinomyces sp. oral taxon 848 str. F0332 Scfld0, whole genome shotgun
sequence; 260447107; NZ_GG703879.1
3958; Actinomyces sp. oral taxon 848 str. F0332 Scfld0, whole genome shotgun
sequence; 260447107; NZ_GG703879.1
3959; Streptomyces ipomoeae 91-03 gcontig_1108499710267, whole genome
shotgun sequence; 429195484; NZ_AEJC01000118.1
3960; Streptomyces ipomoeae 91-03 gcontig_1108499715961, whole genome
shotgun sequence; 429196334; NZ_AEJC01000180.1
3961; Frankia sp. QA3 chromosome, whole genome shotgun sequence;
392941286; NZ_CM001489.1
3962; Fischerella thermalis PCC 7521 contig00099, whole genome shotgun
sequence; 484076371; NZ_AJLL01000098.1
3963; Rhodobacter sp. AKP1 contig19, whole genome shotgun sequence;
429208285; NZ_ANFS01000019.1
3964; Streptomyces chartreusis NRRL 12338 12338 Dorol_scaffold19, whole
genome shotgun sequence; 381200190; NZJH164855.1
3965; Streptomyces globisporus C-1027 Scaffold24_1, whole genome shotgun
sequence; 410651191; NZ_AJU001000171.1
3966; Sphingobium yanoikuyae XLDN2-5 contig000029, whole genome shotgun
sequence; 378759075; NZ_AFXE01000029.1
3967; Paenibacillus peonae KCTC 3763 contig9, whole genome shotgun
sequence; 389822526; NZ_AGFX01000048.1
3968; Citromicrobium sp. JLT1363 contig00009, whole genome shotgun
sequence; 341575924; NZ_AEUE01000009.1
3969; Acaryochloris sp. CCMEE 5410 contig00232, whole genome shotgun
sequence; 359367134; NZ_AFEJ01000154.1
3970; Pseudomonas extremaustralis 14-3 substr. 14-3b strain 14-3 contig00001,
whole genome shotgun sequence; 394743069; NZ_AHIP01000001.1
3971; Lunatimonas lonarensis strain AK24 S14_contig_18, whole genome
shotgun sequence; 499123840; NZ_AQHR01000021.1
3972; Mesorhizobium japonicum R7A MesloDRAFT_Scaffold1.1, whole
genome shotgun sequence; 696358903; NZ_KI632510.1
3973; Legionella pneumophila subsp. pneumophila ATCC 43290, complete
genome; 378775961; NC_016811.1
3974; Methylococcus capsulatus str. Texas = ATCC 19069 strain Texas
contig0129, whole genome shotgun sequence; 483090991;
NZ_AMCE01000064.1
3975; Thermobifida fusca TM51 contig028, whole genome shotgun sequence;
510814910; NZ_AOSG01000028.1
3976; Rhodospirillum rubrum F11, complete genome; 386348020; NC_017584.1
3977; Rhodospirillum rubrum F11, complete genome; 386348020; NC_017584.1
3978; Rhodospirillum rubrum F11, complete genome; 386348020; NC_017584.1
3979; Hahella chejuensis KCTC 2396, complete genome; 83642913; NC_007645.1
3980; Frankia sp. Thr ThrDRAFT_scaffold 28.29, whole genome shotgun
sequence; 602262270; JENI01000029.1
3981; Novosphingobium aromaticivorans DSM 12444, complete genome;
87198026; NC_007794.1
3982; Roseobacter denitnficans OCh 114, complete genome; 110677421; NC_008209.1
3983; Pelobacter propionicus DSM 2379, complete genome; 118578449; NC_008609.1
3984; Psychromonas ingrahamii 37, complete genome; 119943794; NC_008709.1
3985; Rhodobacter sphaeroides ATCC 17029 chromosome 1, complete sequence;
126460778; NC_009049.1
3986; Rhodobacter sphaeroides ATCC 17025, complete genome; 146276058;
NC_009428.1
3987; Geobacter uraniireducens Rf4, complete genome; 148262085; NC_009483.1
3988; Sphingomonas wittichii RW1, complete genome; 148552929; NC_009511.1
3989; Sulfurovum sp. NBC37-1 genomic DNA, complete genome; 152991597;
NC_009663.1
3990; Acaryochloris marina MBIC11017, complete genome; 158333233;
NC_009925.1
3991; Bacillus weihenstephanensis KBAB4, complete genome; 163938013;
NC_010184.1
3992; Bifidobacterium longum subsp infantis ATCC 15697, complete genome;
213690928; NC_011593.1
3993; Cyanothece sp. PCC 7425, complete genome; 220905643; NC_011884.1
3994; Streptococcus suis 98HAH33, complete genome; 145690656; CP000408.1
3995; Chitinophagapinensis DSM 2588, complete genome; 256419057;
NC_013132.1
3996; Rhodothermus marinus DSM 4252, complete genome; 268315578;
NC_013501.1
3997; Sanguibacter keddieii DSM 10542, complete genome; 269793358;
NC_013521.1
3998; Thermobaculum terrenum ATCC AA-798 chromosome 1, complete
sequence; 269925123; NC_013525.1
3999; Thermobaculum terrenum ATCC BAA-798 chromosome 2, complete
sequence; 269838913; NC_013526.1
4000; Xylanimonas cellulosilytica DSM 15894, complete genome; 269954810;
NC_013530.1
4001; Salinibacter ruber M8 chromosome, complete genome; 294505815;
NC_014032.1
4002; Salinibacter ruber M8 chromosome, complete genome; 294505815;
NC_014032.1
4003; Legionella pneumophila 2300/99 Alcoy, complete genome; 296105497;
NC_014125.1
4004; Amycolatopsis mediterranei S699, complete genome; 384145136;
NC_017186.1
4005; Butyrivibrio proteoclasticus B316 chromosome 1, complete sequence;
302669374; NC_014387.1
4006; Gallionella capsifeniformans ES-2, complete genome; 302877245;
NC_014394.1
4007; Paenibacillus polymyxa E681, complete genome; 864439741;
NC_014483.2
4008; Paenibacillus polymyxa 1-43 S143_contig00221, whole genome shotgun
sequence; 647225094; NZ_ASRZ01000173.1
4009; Mesorhizobium ciceri CMG6 MescicDRAFT_scaffold_1.2_C, whole
genome shotgun sequence; 639162053; NZ_AWZS01000002.1
4010; Teniglobus saanensis SP1PR4, complete genome; 320105246;
NC_014963.1
4011; Syntrophobotulus glycolicus DSM 8271, complete genome; 325288201;
NC_015172 .1
4012; Methanobacterium lacus strain AL-21, complete genome; 325957759;
NC_015216.1
4013; Marinomonas mediterranea MMB-1, complete genome; 326793322;
NC_015276.1
4014; Desuffobacca acetoxidans DSM 11109, complete genome; 328951746;
NC_015388.1
4015; Methylomonas methanica MC09, complete genome; 333981747;
B NC_015572.1
4016; Methylomonas methanica MC09, complete genome; 333981747;
NC_015572.1
4017; Methanobacterium paludis strain SWAN1, complete genome; 333986242;
NC_015574.1
4018; Mycobacterium sinense strain JDM601, complete genome; 333988640;
NC_015576.1
4019; Frankia coriariae strain BMG5.1 scaffold41.42, whole genome shotgun
sequence; 827465632; NZ_JWIO01000042.1
4020; Halopiger xanaduensis SH-6 plasmid pHALXA01, complete genome;
336251750; NC_015658.1

4021; Mesorhizobium opportunistum WSM2075, complete genome; 337264537;

NC_015675.1
4022; Runella slithyformis DSM 19594, complete genome; 338209545;
NC_015703.1
4023; Roseobacter litoralis Och 149, complete genome; 339501577;
NC_015730.1
4024; Streptomyces violaceusniger Tu 4113 plasmid pSTRVI01, complete
sequence; 345007457; NC_015951.1
4025; Streptomyces violaceusniger Tu 4113, complete genome; 345007964;
NC_015957.1
4026; Rhodothennus marinus SG0.5JP17-172, complete genome; 345301888;
NC_015966.1
4027; Chloracidobacterium thermophilum B chromosome 1, complete sequence;
347753732; NC_016024.1
4028; Kitasatospora setae KM-6054 DNA, complete genome; 357386972;
NC_016109.1
4029; Streptomyces bingchenggensis BCW-1, complete genome; 374982757;
NC_016582.1
4030; Desulfosporosinus orientis DSM 765, complete genome; 374992780;
NC_016584.1
4031; Desulfosporosinus orientis DSM 765, complete genome; 374992780;
NC_016584.1
4032; Paenibacillus tenae HPL-003, complete genome; 374319880;
NC_016641.1
4033; Bacillus megaterium WSH-002, complete genome; 384044176;
NC_017138.1
4034; Francisella cf. novicida 3523, complete genome; 387823583; NC_017449.1
4035; Streptomyces cattleya str. NRRL 8057 main chromosome, complete
genome; 357397620; NC_016111.1
4036; Streptococcus salivarius JI1V18777 complete genome; 387783149;
NC_017595.1
4037; Actinoplanes sp. SE50/110, complete genome; 386845069; NC_017803.1
4038; Tistrella mobilis KA081020-065 plasmid pTM1, complete sequence;
442559580; NC_017957.2
4039; Tistrella mobilis KA081020-065 plasmid pTM3, complete sequence;
389874236; NC_017958.1
4040; Tistrella mobilis KA081020-065 plasmid pTM3, complete sequence;
389874236; NC_017958.1
4041; Legionella pneumophila subsp. pneumophila str. Lorraine chromosome,
complete genome; 397662556; NC_018139.1
4042; Nocardiopsis sp. TP-A0876 strain NBRC 110039, whole genome shotgun
sequence; 754924215; NZ_BAZE01000001.1
4043; Emticicia oligotrophica DSM 17448, complete genome; 408671769;
NC_018748.1
4044; Saccharothrix espanaensis DSM 44229 complete genome; 433601838;
NC_019673.1
4045; Saccharothrix espanaensis DSM 44229 complete genome; 433601838;
NC_019673.1
4046; Nostoc sp. PCC 7107, complete genome; 427705465; NC_019676.1
4047; Nostoc sp. PCC 7107, complete genome; 427705465; NC_019676.1
4048; Rivularia sp. PCC 7116, complete genome; 427733619; NC_019678.1
4049; Rivularia sp. PCC 7116, complete genome; 427733619; NC_019678.1
4050; Synechococcus sp. PCC 6312, complete genome; 427711179;
NC_019680.1
4051; Synechococcus sp. PCC 6312, complete genome; 427711179;
NC_019680.1
4052; Nostoc sp. PCC 7524, complete genome; 427727289; NC_019684.1
4053; Calothrix sp. PCC 6303, complete genome; 428296779; NC_019751.1
4054; Crinalium epipsammum PCC 9333, complete genome; 428303693;
NC_019753.1
4055; Thermobacillus composti KWC4, complete genome; 430748349;
NC_019897.1
4056; Mesorhizobium sp. LNHC220B00 scaffold0002, whole genome shotgun
sequence; 563576979; NZ_AYWS01000002.1
4057; Bacillus sp. 1NLA3E, complete genome; 488570484; NC_021171.1
4058; Streptomyces davawensis strain JCM 4913 complete genome; 471319476;
NC_020504.1
4059; Streptomyces davawensis strain JCM 4913 complete genome; 471319476;
NC_020504.1
4060; Desulfotomaculum acetoxidans DSM 771, complete genome; 258513366;
NC_013216.1
4061; Desulfotomaculum acetoxidans DSM 771, complete genome; 258513366;
NC_013216.1
4062; Actinosynnema mirum DSM 43827, complete genome; 256374160;
NC_013093.1
4063; Bacillus cereus BAG20-3 acfXF-supercont1.1, whole genome shotgun
sequence; 507017505; NZ_KB976530.1
4064; Bacillus cereus HuA3-9 acqVv-supercont1.4, whole genome shotgun
sequence; 507024338; NZ_KB976146.1
4065; Bacillus cereus VD118 acrHo-supercont1.9, whole genome shotgun
sequence; 507035131; NZ_KB976800.1
4066; Bacillus cereus VDM006 acrHb-supercont1.1, whole genome shotgun
sequence; 507060269; NZ_KB976864.1
4067; Bacillus cereus VDM019 acluj-supercont1.2, whole genome shotgun
sequence; 507056808; NZ_KB976199.1
4068; Bacillus cereus VDM053 acrGS-supercont1.7, whole genome shotgun
sequence; 507060152; NZ_KB976714.1
4069; Halomonas anticaliensis FP35 = DSM 16096 strain FP35 Scaffold 1, whole
genome shotgun sequence; 514429123; NZ_KE332377.1
4070; Streptomyces sp. NRRL F-5917 contig68.1, whole genome shotgun
sequence; 663414324; NZ_JOHQ01000068.1
4071; Streptomyces aurantiacus JA 4570 Seq17, whole genome shotgun sequence;
514916021; NZ_AOPZ01000017.1
4072; Streptomyces aurantiacus JA 4570 Seq63, whole genome shotgun sequence;
514917321; NZ_AOPZ01000063.1
4073; Streptomyces aurantiacus JA 4570 Seq109, whole genome shotgun
sequence; 514918665; NZ_AOPZ01000109.1
4074; Paenibacillus polymyxa OSY-DF Contig136, whole genome shotgun
sequence; 484036841; NZ_AIPP01000136.1
4075; Fischerella muscicola SAG 1427-1 = PCC 73103 contig00215, whole
genome shotgun sequence; 484073367; NZ_AJLJ01000207.1
4076; Fischerella muscicola PCC 7414 contig00109, whole genome shotgun
sequence; 484075173; NZ_AJLK01000109.1
4077; Fischerella muscicola PCC 7414 contig00153, whole genome shotgun
sequence; 484075372; NZ_AJLK01000153.1
4078; Pedobacter arcticus A12 Scaffold2, whole genome shotgun sequence;
484345004; NZ_JH947126.1
4079; Leptolyngbyaboryana PCC 6306 LepboDRAFT LPC.1, whole genome
shotgun sequence; 482909028; NZ_KB731324.1
4080; Mastigocladus laminosus UU774 scaffold 22, whole genome shotgun
sequence; 764671177; NZ_JX1101000139.1
4081; Fischerella sp. PCC 9339 PCC9339DRAFT_scaffold1.1, whole genome
shotgun sequence; 482909394; NZ_JI4992898.1
4082; Lactococcus garvieae Tac2 Tac2Contig_33, whole genome shotgun
sequence; 483258918; NZ_AMFE01000033.1
4083; Paenisporosarcina sp. TG-14 111.TG14.1_1, whole genome shotgun
sequence; 483299154; NZ_AMGD01000001.1
4084; Paenibacillus sp. ICGEB2008 Contig_7, whole genome shotgun sequence;
483624383; NZ_AMQUO1000007.1
4085; Amphibacillus jilinensis Y1 Scaffold2, whole genome shotgun sequence;
483992405; NZ__M976435.1
4086; Nocardiopsis alba DSM 43377 contig 34, whole genome shotgun
sequence; 484007204; NZ_ANAC01000034.1
4087; Nocardiopsis halophila DSM 44494 contig 138, whole genome shotgun
sequence; 484007841; NZ_ANAD01000138.1
4088; Nocardiopsis halophila DSM 44494 contig 138, whole genome shotgun
sequence; 484007841; NZ_ANAD01000138.1
4089; Nocardiopsis halophila DSM 44494 contig 197, whole genome shotgun
sequence; 484008051; NZ_ANAD01000197.1
4090; Nocardiopsis baichengensis YIM 90130 Scaffold15_1, whole genome
shotgun sequence; 484012558; NZ_ANAS01000033.1
4091; Nocardiopsis halotolerans DSM 44410 contig 26, whole genome shotgun
sequence; 484015294; NZ_ANAX01000026.1
4092; Nocardiopsis salina YIM 90010 contig 204, whole genome shotgun
sequence; 484023808; NZ_ANBF01000204.1
4093; Nocardiopsis chromatogenes YIM 90109 contig 59, whole genome
shotgun sequence; 484026076; NZ_ANBH01000059.1
4094; Nocardiopsis chromatogenes YIM 90109 contig 93, whole genome
shotgun sequence; 484026206; NZ_ANBH01000093.1
4095; Porphyrobacter sp. AAP82 Contig35, whole genome shotgun sequence;
484033307; NZ_ANFX01000035.1
4096; Blastomonas sp. AAP53 Contig14, whole genome shotgun sequence;
484033631; NZ_ANFZ01000014.1
4097; Paenibacillus sp. PAMC 26794 5104_29, whole genome shotgun sequence;
484070054; NZ_ANHX01000029.1
4098; Oscillatoria sp. PCC 10802 Osc10802DRAFT_Contig7.7, whole genome
shotgun sequence; 484104632; NZ_KB235948.1
4099; Clostridium botulinum CB11/1-1 CB contig00105, whole genome shotgun
sequence; 484141779; NZ_AORM01000006.1
4100; Actinopolyspora halophila DSM 43834 ActhaDRAFT_contig1.1_C, whole
genome shotgun sequence; 484203522; NZ_AQUI01000002.1
4101; Streptomyces sp. FxanaC1 B074DRAFT scaffold_1.2_C, whole genome
shotgun sequence; 484227180; NZ_AQW001000002.1
4102; Smaragdicoccus niigatensis DSM 44881 = NBRC 103563 strain DSM
44881 F600DRAFT_scaffold00011.11_C, whole genome shotgun sequence;
484234624; NZ_AQXZ01000009.1
4103; Verrucomicrobium sp. 3C A37ADRAFT_scaffold1.1, whole genome
shotgun sequence; 483219562; NZ_KB901875.1
4104; Verrucomicrobium sp. 3C A37ADRAFT_scaffold1.1, whole genome
shotgun sequence; 483219562; NZ_KB901875.1
4105; Ancylobacter sp. FA202 A3M1DRAFT_scaffold1.1, whole genome
shotgun sequence; 483720774; NZ_KB904818.1
4106; Filamentous cyanobacterium ESFC-1 A3MYDRAFT_scaffold1.1, whole
genome shotgun sequence; 483724571; NZ_KB904821.1
4107; Streptomyces sp. CcalMP-8W B053DRAFT_scaffold_17.18, whole
genome shotgun sequence; 483961830; NZ_KB890924.1
4108; Streptomyces sp. ScaeMP-e10 B061DRAFT_scaffold_01, whole genome
shotgun sequence; 483967534; NZ_KB891296.1
4109; Streptomyces sp. CNB091 D581DRAFT_scaffold00010.10, whole genome
shotgun sequence; 484070161; NZ_KB898999.1
4110; Streptomyces sp. T0R3209 Contig613, whole genome shotgun sequence;
484867902; NZ_AGNH01000613.1
4111; Bacillus oceanisediminis 2691 contig2644, whole genome shotgun
sequence; 485048843; NZ_ALEG01000067.1
4112; Bacillus sp. REN51N contig 2, whole genome shotgun sequence;
748816024; NZ_JXAB01000002.1
4113; Calothrix sp. PCC 7103 Ca17103DRAFT_CPM.6, whole genome shotgun
sequence; 485067373; NZ_KB217478.1
4114; Pseudanabaena sp. PCC 6802 Pse6802_scaffold_5, whole genome shotgun
sequence; 485067426; NZ_KB235914.1
4115; Actinomadura atramentaria DSM 43919 strain SF2197
G339DRAFT_scaffold00002.2, whole genome shotgun sequence; 485090585;
NZ_KB907209.1
4116; Novispirillum itersonii subsp. itersonii ATCC 12639
G365DRAFT_scaffold00001.1, whole genome shotgun sequence; 485091510;
NZ_KB907337.1
4117; Novispirillum itersonii subsp. itersonii ATCC 12639
G365DRAFT_scaffold00001.1, whole genome shotgun sequence; 485091510;
NZ_KB907337.1
4118; Paenibacillus polymyxa ATCC 842 PPt02_scaffold1, whole genome
shotgun sequence; 485269841; NZ_GL905390.1
4119; Streptomyces sp. SolWspMP-so12th B083DRAFT_scaffold_17.18_C,
whole genome shotgun sequence; 654969845; NZ_ARPF01000020.1
4120; Mesorhizobium hualcuii 7653R genome; 657121522; CP006581.1
4121; Paenibacillus sp. FIW567 B212DRAFT_scaffold1.1, whole genome
shotgun sequence; 486346141; NZ_KB910518.1
4122; Bacillus sp. 123MFChir2 H280DRAFT_scaffold00030.30, whole genome
shotgun sequence; 487368297; NZ_KB910953.1
4123; Streptomyces canus 299MFChir4.1 H293DRAFT_scaffold00032.32, whole
genome shotgun sequence; 487385965; NZ_KB911613.1
4124; Nocardiopsis potens DSM 45234 contig 25, whole genome shotgun
sequence; 484017897; NZ_ANBB01000025.1
4125; Kribbella catacumbae DSM 19601 A3ESDRAFT_scaffold_7.8S, whole
genome shotgun sequence; 484207511; NZ_AQUZ01000008.1
4126; Paenibacillus riograndensis SBR5 Contig78, whole genome shotgun
sequence; 485470216; NZ_A
4127; Lamprocystis purpurea DSM 4197 A390DRAFT_scaffold_01, whole
genome shotgun sequence; 483254584; NZ_KB902362.1
4128; Nonomumea coxensis DSM 45129 A3G7DRAFT_scaffold_4.5, whole
genome shotgun sequence; 483454700; NZ_KB903974.1
4129; Spirosoma spitsbergense DSM 19989 B157DRAFT_scaffold_76.77, whole
genome shotgun sequence; 483994857; NZ_KB893599.1
4130; Amycolatopsis benzoatilytica AK 16/65 AmybeDRAFT_scaffold1.1, whole
genome shotgun sequence; 486399859; NZ_KB912942.1
4131; Amycolatopsis nigrescens CSC17Ta-90 AmyniDRAFT Contig68.1_C,
whole genome shotgun sequence; 487404592; NZ_ARVW01000001.1
4132; Amycolatopsis nigrescens CSC17Ta-90 AmyniDRAFT Contig68.1S,
whole genome shotgun sequence; 487404592; NZ_ARVW01000001.1
4133; Reyranella massiliensis 521, whole genome shotgun sequence; 484038067;
NZ_HE997181.1
4134; Acidobacteriaceae bacterium KBS 83 GO02DRAFT scaffold00007.7,
whole genome shotgun sequence; 485076323; NZ_KB906739.1
4135; Paenibacillus alvei A6-6i-x PAAL66ix_14, whole genome shotgun
sequence; 528200987; ATMS01000061.1
4136; Dehalobacter sp. UNSWDHB Contig 139, whole genome shotgun
sequence; 544905305; NZ_AUUR01000139.1
4137; Thermoactinomyces vulgaris strain NRRL F-5595 F5595contig15.1, whole
genome shotgun sequence; 929862756; NZ_LGKI01000090.1
4138; Clostridium saccharobutylicum DSM 13864, complete genome;
550916528; NC_022571.1
4139; Butyrivibrio fibrisolvens AB2020 G616DRAFT scaffold00015.15_C,
whole genome shotgun sequence; 551012921; NZ_ATVZ01000015.1
4140; Butyrivibrio sp. XPD2006 G590DRAFT scaffold00008.8S, whole
genome shotgun sequence; 551021553; NZ_ATVT01000008.1
4141; Acidobacteriaceae bacterium TAA166 strain TAA 166
H979DRAFT scaffold 0.1S, whole genome shotgun sequence; 551216990;
NZ_ATWD01000001.1
4142; Acidobacteriaceae bacterium TAA166 strain TAA 166
H979DRAFT scaffold 0.1S, whole genome shotgun sequence; 551216990;
NZ_ATWD01000001.1
4143; Leptolyngbya sp. Heron Island J 50, whole genome shotgun sequence;
553739852; NZ_AWNH01000066.1
4144; Leptolyngbya sp. Heron Island J 50, whole genome shotgun sequence;
553739852; NZ_AWNH01000066.1
4145; Leptolyngbya sp. Heron Island J 67, whole genome shotgun sequence;
553740975; NZ_AWNH01000084.1
4146; Rothia aeria F0184 R aeriaFIMPREF0742-1.0_Cont136.4, whole genome
shotgun sequence; 551695014; AXZGO1000035.1
4147; Gloeobacter kilaueensis JS1, complete genome; 554634310; NC_022600.1
4148; Gloeobacter kilaueensis JS1, complete genome; 554634310; NC_022600.1
4149; Asticcacaulis sp. AC466 contig00033, whole genome shotgun sequence;
557835508; NZ_AWGE01000033.1
4150; Streptomyces niveus NCIMB 11891 contig00003, whole genome shotgun
sequence; 558542923; AWQW01000003.1
4151; Streptomyces roseochromogenus subsp. oscitans DS 12.976 chromosome,
whole genome shotgun sequence; 566155502; NZ_CM002285.1
4152; Streptomyces roseochromogenus subsp. oscitans DS 12.976 chromosome,
whole genome shotgun sequence; 566155502; NZ_CM002285.1
4153; Bacillus boroniphilus JCM 21738 DNA, contig: contig_6, whole genome
shotgun sequence; 571146044; BAUW01000006.1
4154; Mesorhizobium sp. LSJC285A00 scaffold0007, whole genome shotgun
sequence; 563442031; NZ_AYVK01000007.1
4155; Mesorhizobium sp. LSJC277A00 scaffold0014, whole genome shotgun
sequence; 563459186; NZ_AYVM01000014.1
4156; Mesorhizobium sp. LNJC405B00 scaffold0005, whole genome shotgun
sequence; 563523441; NZ_AYWC01000005.1
4157; Mesorhizobium sp. LSJC265A00 scaffold0015, whole genome shotgun
sequence; 563472037; NZ_AYVP01000015.1
4158; Mesorhizobium sp. LSHC426A00 scaffold0005, whole genome shotgun
sequence; 563492715; NZ_AYVV01000005.1
4159; Mesorhizobium sp. LNHC232B00 scaffold0020, whole genome shotgun
sequence; 563561985; NZ_AYWP01000020.1
4160; Mesorhizobium sp. L48CO26A00 scaffold0030, whole genome shotgun
sequence; 563848676; NZ_AYWU01000030.1
4161; Mesorhizobium sp. L2C089B000 scaffold0011, whole genome shotgun
sequence; 563888034; NZ_AYWV01000011.1
4162; Mesorhizobium sp. L2C084A000 scaffold0007, whole genome shotgun
sequence; 563938926; NZ_AYWX01000007.1
4163; Closltidium pasteurianum NRRL B-598, complete genome; 930593557;
NZ_CP011966.1
4164; Paenibacillus polymyxa CR1, complete genome; 734699963; NC_023037.2
4165; Closltidium butyricum DORA 1 Q607 CBUC00058, whole genome
shotgun sequence; 566226100; AZLX01000058.1
4166; Streptococcus suis strain LS8F, whole genome shotgun sequence;
766589647; NZ_CEHJ01000007.1
4167; Mycobacterium sp. UM Kg27 contig000002, whole genome shotgun
sequence; 809025315; NZ_JRMM01000002.1
4168; Mycobacterium iranicum UM TJL Contig 42, whole genome shotgun
sequence; 638987534; NZ_AUWT01000042.1
4169; Paenibacillus sp. MAEPY2 contig7, whole genome shotgun sequence;
639451286; NZ_AWUK01000007.1
4170; Verrucomicrobia bacterium LP2A G346DRAFT_scf7180000000012_quiver.2_C,
whole genome shotgun sequence; shotgun sequence;
640169055; NZ_JAFS01000002.1
4171; Verrucomicrobia bacterium LP2A G346DRAFT_scf7180000000012_quiver.2_C,
whole genome shotgun sequence; 640169055; NZ_JAFS01000002.1
4172; Bacillus mannanilyticus JCM 10596, whole genome shotgun sequence;
640600411; NZ_BAM001000071.1
4173; Bifidobacterium breve NCFB 2258, complete genome; 749295448;
NZ_CP006714.1
4174; Haloglycomyces albus DSM 45210 HalaIDRAFT_chromosome1.1_C,
whole genome shotgun sequence; 644043488; NZ_AZUQ01000001.1
4175; Kutzneria albida strain NRRL B-24060 contig305.1, whole genome shotgun
sequence; 662161093; NZ_JNYHO1000515.1
4176; Kutzneria albida strain NRRL B-24060 contig305.1, whole genome shotgun
sequence; 662161093; NZ_JNYH01000515.1
4177; Kutzneria albida DSM 43870, complete genome; 754862786;
NZ_CP007155.1
4178; Paenibacillus sp. 1-49 S149_contig00281, whole genome shotgun sequence;
647230448; NZ_ASRY01000102.1
4179; Paenibacillus graminis RSA19 S2_contig00597, whole genome shotgun
sequence; 647256651; NZ_ASSG01000304.1
4180; Paenibacillus sp. 1-18 S118_contig00103, whole genome shotgun sequence;
647269417; NZ_ASSB01000031.1
4181; Paenibacillus polymyxa TD94 STD94_contig00759, whole genome
shotgun sequence; 647274605; NZ_ASSA01000134.1
4182; Bacillus flexus T6186-2 contig 106, whole genome shotgun sequence;
647636934; NZ_JANV01000106.1
4183; Mastigocladopsis repens PCC 10914 Mas10914DRAFT_scaffold1.1, whole
genome shotgun sequence; 482909462; NZJH992901.1
4184; Streptomyces sp. FxanaC1 B074DRAFT_scaffold_7.8_C, whole genome
shotgun sequence; 484227195; NZ_AQW001000008.1
4185; Streptomyces sp. HmicAl2 B072DRAFT_scaffold_19.20, whole genome
shotgun sequence; 483972948; NZ_KB891808.1
4186; Butyrivibrio sp. XPD2002 G587DRAFT_scaffold00011.11, whole genome
shotgun sequence; 651381584; NZ_KE384117.1
4187; Butyrivibrio sp. NC3005 G634DRAFT scaffold00001.1, whole genome
651394394; NZ_KE384206.1
4188; Paenarthrobacter nicotinovorans 231Sha2.1M6I960DRAFT_scaffold00004.4_C,
whole genome shotgun sequence; 651445346; NZ_AZVC01000006.1
4189; Bacillus sp. J37 BacJ37DRAFT_scaffold_0.1_C, whole genome shotgun
sequence; 651516582; NZ_JAEK01000001.1
4190; Bacillus sp. UNC437CL72CviS29 M014DRAFT_scaffold00009.9_C,
whole genome shotgun sequence; 651596980; NZ_AXVB01000011.1
4191; Bacillus bogoriensis ATCC BAA-922 T323DRAFT_scaffold00008.8_C,
whole genome shotgun sequence; 651937013; NZJHYI01000013.1
4192; Bacillus kribbensis DSM 17871 H539DRAFT_scaffold00003.3, whole
genome shotgun sequence; 651983111; NZ_KE387239.1
4193; Fischerella sp. PCC 9431 Fis9431DRAFT_Scaffold1.2, whole genome
shotgun sequence; 652326780; NZ_KE650771.1
4194; Fischerella sp. PCC 9605 FIS9605DRAFT_scaffold2.2, whole genome
shotgun sequence; 652337551; NZ_K1912149.1
4195; Clostridium akagii DSM 12554 BR66DRAFT_scaffold00010.10_C, whole
genome shotgun sequence; 652488076; NZ_JMLK01000014.1
4196; Closltidium beijerinckii HUN142 T483DRAFT_scaffold00004.4, whole
genome shotgun sequence; 652494892; NZ_KK211337.1
4197; Mesorhizobium sp. URHA0056 H959DRAFT scaffold00004.4_C, whole
genome shotgun sequence; 652670206; NZ_AUEL01000005.1
4198; Mesorhizobium loti R88b Meslo2DRAFT_Scaffold1.1, whole genome
shotgun sequence; 652688269; NZ_KI912159.1
4199; Mesorhizobium ciceri W5M4083 MESCI2DRAFT_scaffold_0.1, whole
genome shotgun sequence; 652698054; NZ_KI912610.1
4200; Mesorhizobium sp. URHC0008 N549DRAFT_scaffold00001.1_C, whole
genome shotgun sequence; 652699616; NZ_JIAP01000001.1
4201; Mesorhizobium erdmanii USDA 3471 A3AUDRAFT_scaffold_7.8_C,
whole genome shotgun sequence; 652719874; NZ_AXAE01000013.1
4202; Mesorhizobium loti CJ3sym A3A9DRAFT_scaffold 25.26_C, whole
genome shotgun sequence; 652734503; NZ_AXAL01000027.1
4203; Cohnella thermotolerans DSM 17683 G485DRAFT_scaffold00041.41_C,
whole genome shotgun sequence; 652787974; NZ_AUCP01000055.1
4204; Mesorhizobium sp. WSM3626 Mesw3626DRAFT_scaffold_6.7_C, whole
genome shotgun sequence; 652879634; NZ_AZUY01000007.1
4205; Mesorhizobium sp. W5M1293 MesloDRAFT scaffold 4.5, whole genome
shotgun sequence; 652910347; NZ_KI911320.1
4206; Mesorhizobium sp. W5M3224 YU3DRAFT_scaffold_3.4_C, whole
genome shotgun sequence; 652912253; NZ_ATY001000004.1
4207; Butyrivibrio fibrisolvens MD2001 G635DRAFT_scaffold00033.33_C,
whole genome shotgun sequence; 652963937; NZ_AUKDO1000034.1
4208; Legionella pneumophila subsp. pneumophila strain ATCC 33155
contig032, whole genome shotgun sequence; 652971687; NZ_JFIN01000032.1
4209; Legionella pneumophila subsp. pneumophila strain ATCC 33154 5caffold2,
whole genome shotgun sequence; 653016013; NZ_KK074241.1
4210; Legionella pneumophila subsp. pneumophila strain ATCC 33823 5caffold7,
whole genome shotgun sequence; 653016661; NZ_KK074199.1
4211; Bacillus sp. URHB0009 H980DRAFT_scaffold00016.16_C, whole
genome shotgun sequence; 653070042; NZ_AUER01000022.1
4212; Lachnospira multipara ATCC 19207 G600DRAFT_scaffold00009.9_C,
whole genome shotgun sequence; 653218978; NZ_AUJG01000009.1
4213; Streptomyces sp. CNH099 B121DRAFT_scaffold_16.17_C, whole
genome shotgun sequence; 654239557; NZ_AZWL01000018.1
4214; Desulfatiglans anilini DSM 4660 H567DRAFT_scaffold00005.5_C, whole
genome shotgun sequence; 654868823; NZ_AULM01000005.1
4215; Legionella pneumophila subsp. fraseri strain ATCC 35251 contig031, whole
genome shotgun sequence; 654928151; NZ_JFIG01000031.1
4216; Bacillus sp. FJAT-14578 5caffold2, whole genome shotgun sequence;
654948246; NZ_K1632505.1
4217; Bacillus sp. 278922_107 H622DRAFT_scaffold00001.1, whole genome
shotgun sequence; 654964612; NZ_KI911354.1
4218; Ruminococcus flavefaciens ATCC 19208 L870DRAFT_scaffold00001.1,
whole genome shotgun sequence; 655069822; NZ_KI912489.1
4219; Paenibacillus taiwanensis DSM 18679 H.509DRAFT_scaffold00010.10_C,
whole genome shotgun sequence; 655095554; NZ_AULE01000001.1
4220; Paenibacillus sp. UNC451MF BP97DRAFT_scaffold00018.18_C, whole
genome shotgun sequence; 655103160; NZ_JMLS01000021.1
4221; Paenibacillus pinihumi DSM 23905 = JCM 16419 strain DSM 23905
H583DRAFT_scaffold00005.5, whole genome shotgun sequence; 655115689;
NZ_KE383867.1
4222; Paenibacillus harenae DSM 16969 H581DRAFT scaffold00002.2, whole
genome shotgun sequence; 655165706; NZ_KE383843.1
4223; Shimazuella kribbensis DSM 45090 A3GQDRAFT_scaffold_0.1S, whole
genome shotgun sequence; 655370026; NZ_ATZFO1000001.1
4224; Shimazuella kribbensis DSM 45090 A3GQDRAFT_scaffold_5.6_C, whole
genome shotgun sequence; 655371438; NZ_ATZFO1000006.1
4225; Streptomyces flavidovirens DSM 40150 G412DRAFT_scaffold00007.7_C,
whole genome shotgun sequence; 655414006; NZ_AUBE01000007.1
4226; Streptomyces flavidovirens DSM 40150 G412DRAFT_scaffold00009.9,
whole genome shotgun sequence; 655416831; NZ_KE386846.1
4227; Azospirillum halopraeferens DSM 3675 G472DRAFT_scaffold00039.39_C,
whole genome shotgun sequence; 655967838; NZ_AUCF01000044.1
4228; Closltidium scatologenes strain ATCC 25775, complete genome;
802929558; NZ_CP009933.1
4229; Paenibacillus harenae DSM 16969 H581DRAFT_scaffold00004.4, whole
genome shotgun sequence; 656245934; NZ_KE383845.1
4230; Paenibacillus alginolyticus DSM 5050 = NBRC 15375 strain DSM 5050
G519DRAFT_scaffold00043.43_C, whole genome shotgun sequence;
656249802; NZ_AUGY01000047.1
4231; Paenibacillus alginolyticus DSM 5050 = NBRC 15375 strain DSM 5050
G519DRAFT_scaffold00043.43_C, whole genome shotgun sequence;
656249802; NZ_AUGY01000047.1
4232; Bacillus indicus strain DSM 16189 Contig01, whole genome shotgun
sequence; 737222016; NZ_JNVCO2000001.1
4233; Bacillus sp. RP1137 contig 18, whole genome shotgun sequence;
657210762; NZ_AXZS01000018.1
4234; Streptomyces leeuwenhoekii strain C34(2013) c34_sequence_0012, whole
genome shotgun sequence; 657294764; NZ_AZSD01000012.1
4235; Streptomyces leeuwenhoekii strain C34(2013) c34_sequence_0041, whole
genomeshotgun sequence; 657295264; NZ_AZSD01000040.1
4236; Streptomyces leeuwenhoekii strain C58 contig69, whole genome shotgun
sequence; 873282617; NZ_LFEH01000068.1
4237; Bacillus thuringiensis LM1212 scaffold 08, whole genome shotgun
sequence; 657629081; NZ_AYPV01000024.1
4238; Paenibacillus polymyxa strain WLY78 S6_contig00095, whole genome
shotgun sequence; 657719467; NZ_ALN01000094.1
4239; [Scytonema hofmanni] UTEX 2349 To19009DRAFT_TPD.8, whole
genome shotgun sequence; 657935980; NZ_KK073768.1
4240; Sphingomonas sp. DC-6 scaffold87, whole genome shotgun sequence;
662140302; NZ_JMUB01000087.1
4241; Streptomyces lavendulae strain Fujisawa #8006 contig417.1, whole genome
shotgun sequence; 662043624; NZ_JNXL01000469.1
4242; Streptomyces sp. NRRL WC-3773 contig36.1, whole genome shotgun
sequence; 664487325; NZ_J01101000036.1
4243; Streptomyces flavotricini strain NRRL B-5419 contig237.1, whole genome
shotgun sequence; 662063073; NZ_JNXV01000303.1
4244; Streptomyces peruviensis strain NRRL ISP-5592 P181_Doro1_scaffold152,
whole genome shotgun sequence; 662097244; NZ_KL575165.1
4245; Streptomyces natalensis ATCC 27448 Scaffold 33, whole genome shotgun
sequence; 764439507; NZ_JRKI01000027.1
4246; Streptomyces decoyicus strain NRRL ISP-5087 P056_Doro1_scaffold78,
whole genome shotgun sequence; 662133033; NZ_KL570321.1
4247; Streptomyces baamensis strain NRRL B-2842 P144_Doro1_scaffold26,
whole genome shotgun sequence; 662135579; NZ_KL573564.1
4248; Streptomyces vinaceus strain NRRL ISP-5257 contig5.1, whole genome
shotgun sequence; 759527818; NZ_JNYP01000005.1
4249; Spirillospora albida strain NRRL B-3350 contig1.1, whole genome shotgun
sequence; 663122276; NZ_JOFJ01000001.1
4250; Streptomyces sp. NRRL S-455 contig1.1, whole genome shotgun sequence;
663192162; NZ_JOCT01000001.1
4251; Streptomyces sp. NRRL S-87 contig69.1, whole genome shotgun sequence;
663169513; NZ_JO
4252; Streptomyces katrae strain NRRL B-16271 contig33.1, whole genome
shotgun sequence; 663300513; NZ_JNZY01000033.1
4253; Streptomyces katrae strain NRRL B-16271 contig37.1, whole genome
shotgun sequence; 663300941; NZ_JNZY01000037.1
4254; Streptomyces sp. NRRL B-3229 contig5.1, whole genome shotgun
sequence; 663316931; NZ_JOGP01000005.1
4255; Streptomyces ruber strain NRRL B-1661 contig94.1, whole genome
shotgun sequence; 663365281; NZ_JODN01000094.1
4256; Streptomyces roseoverticillatus strain NRRL B-3500 contig22.1, whole
genome shotgun sequence; 663372343; NZ_JOFL01000022.1
4257; Streptomyces roseoverticillatus strain NRRL B-3500 contig43.1, whole
genome shotgun sequence; 663373497; NZ_JOFL01000043.1
4258; Streptomyces rimosus subsp. rimosus strain NRRL WC-3869
P248contig20.1, whole genome shotgun sequence; 925322461;
LGCQ01000113.1
4259; Streptomyces sp. NRRL B-12105 contig1.1, whole genome shotgun
sequence; 663380895; NZ_JNZW01000001.1
4260; Streptomyces sp. NRRL S-1448 contig134.1, whole genome shotgun
sequence; 663421576; NZ_JOGE01000134.1
4261; Allokutzneria albata strain NRRL B-24461 contig22.1, whole genome
shotgun sequence; 663596322; NZ_JOEF01000022.1
4262; Herbidospora cretacea strain NRRL B-16917 contig7.1, whole genome
shotgun sequence; 663670981; NZ_JODQ01000007.1
4263; Nocardia sp. NRRL WC-3656 contig2.1, whole genome shotgun sequence;
663737675; NZ_JOJF01000002.1
4264; Streptomyces aureocirculatus strain NRRL ISP-5386 contig11.1, whole
genome shotgun sequence; 664013282; NZ_JOAP01000011.1
4265; Streptomyces cyaneofuscatus strain NRRL B-2570 contig9.1, whole
genome shotgun sequence; 664021017; NZ_JOEM01000009.1
4266; Streptomyces aureocirculatus strain NRRL ISP-5386 contig49.1, whole
genome shotgun sequence; 664026629; NZ_JOAP01000049.1
4267; Streptomyces sclerotialus strain NRRL B-2317 contig7.1, whole genome
shotgun sequence; 664034500; NZ_JODX01000007.1
4268; Streptomyces anulatus strain NRRL B-2873 contig21.1, whole genome
shotgun sequence; 664049400; NZ_JOEZ01000021.1
4269; Streptomyces globisporus subsp. globisporus strain NRRL B-2709
contig24.1, whole genome shotgun sequence; 664051798; NZ_JNZK01000024.1
4270; Streptomyces rimosus subsp. rimosus strain NRRL B-2660 contig14.1,
whole genome shotgun sequence; 664052786; NZ_JOES01000014.1
4271; Streptomyces achromogenes subsp. achromogenes strain NRRL B-2120
contig2.1, whole genome shotgun sequence; 664063830; NZ_JODT01000002.1
4272; Streptomyces rimosus subsp. rimosus strain NRRL B-2660 contig124.1,
whole genome shotgun sequence; 664066234; NZ_JOES01000124.1
4273; Streptomyces rimosus subsp. rimosus strain NRRL WC-3927 contig5.1,
whole genome shotgun sequence; 664091759; NZ_JOB001000005.1
4274; Streptomyces rimosus subsp. rimosus strain NRRL WC-3904 contig10.1,
whole genome shotgun sequence; 664126885; NZ_JOCQ01000010.1
4275; Streptomyces rimosus subsp. rimosus strain NRRL WC-3904 contig106.1,
whole genome shotgun sequence; 664141810; NZ_JOCQ01000106.1
4276; Streptomyces sp. NRRL F-2295 P395contig79.1, whole genome shotgun
sequence; 926288193; NZ_LGCY01000146.1
4277; Streptomyces lavenduligriseus strain NRRL ISP-5487 contig2.1, whole
genome shotgun sequence; 664244706; NZ_JOBD01000002.1
4278; Streptomyces lavenduligriseus strain NRRL ISP-5487 contig2.1, whole
genome shotgun sequence; 664244706; NZ_JOBD01000002.1
4279; Streptomyces alboniger strain NRRL B-1832 B-1832_contig_384, whole
genome shotgun sequence; 943899498; NZ_LIQN01000384.1
4280; Streptomyces sp. NRRL S-337 contig31.1, whole genome shotgun
sequence; 664275807; NZ_JOIX01000031.1
4281; Streptomyces sp. NRRL S-337 contig41.1, whole genome shotgun
sequence; 664277815; NZ_JOIX01000041.1
4282; Streptomyces hygroscopicus subsp. hygroscopicus strain NRRL B-1477
contig8.1, whole genome shotgun sequence; 664299296; NZ_JOIK01000008.1
4283; Streptomyces sp. NRRL F-4474 contig32.1, whole genome shotgun
sequence; 664323078; NZ_JOIB01000032.1
4284; Streptomyces sp. NRRL S-475 contig32.1, whole genome shotgun
sequence; 664325162; NZ_JOJB01000032.1
4285; Streptomyces sp. NRRL S-1868 contig54.1, whole genome shotgun
sequence; 664360925; NZ_JOGD01000054.1
4286; Streptomyces sp. NRRL S-646 contig23.1, whole genome shotgun
sequence; 664421883; NZ_JODC01000023.1
4287; Streptomyces sp. NRRL S-1813 contig13.1, whole genome shotgun
sequence; 664466568; NZ_JOHB01000013.1
4288; Streptomyces sp. NRRL WC-3773 contig2.1, whole genome shotgun
sequence; 664478668; NZ_JOJI01000002.1
4289; Streptomyces sp. NRRL WC-3773 contig11.1, whole genome shotgun
sequence; 664481891; NZ_JOJI01000011.1
4290; Streptomyces sp. NRRL WC-3773 contig36.1, whole genome shotgun
sequence; 664487325; NZ_JOJI01000036.1
4291; Streptomyces olivaceus strain NRRL B-3009 contig20.1, whole genome
shotgun sequence; 664523889; NZ_JOFH01000020.1
4292; Streptomyces sp. NRRL F-5702 contig3.1, whole genome shotgun
sequence; 664537198; NZ_JOHD01000003.1
4293; Streptomyces ochmceiscleroticus strain NRRL ISP-5594 contig9.1, whole
genome shotgun sequence; 664540649; NZ_JOAX01000009.1
4294; Streptomyces sp. NRRL S-118 P205_Doro1_scaffold2, whole genome
shotgun sequence; 664556736; NZ_KL591003.1
4295; Streptomyces sp. NRRL WC-3641 P206 Dorol_scaffold18, whole
genome shotgun sequence; 664607641; NZ_KL579016.1
4296; Streptomyces sp. NRRL S-623 contig14.1, whole genome shotgun
sequence; 665522165; NZ_JOJC01000016.1
4297; Streptomyces sp. NRRL WC-3719 contig152.1, whole genome shotgun
sequence; 665536304; NZ_JOCD01000152.1
4298; Streptomyces durhamensis strain NRRL B-3309 contig3.1, whole genome
shotgun sequence; 665586974; NZ_JNXR01000003.1
4299; Streptomyces durhamensis strain NRRL B-3309 contig23.1, whole genome
shotgun sequence; 665604093; NZ_JNXR01000023.1
4300; Bacillus sp. MB2021 T349DRAFT_scaffold00010.10_C, whole genome
shotgun sequence; 671553628; NZ_JN1101000011.1
4301; Lachnospira multipara LB2003 T537DRAFT_scaffold00010.10_C, whole
genome shotgun sequence; 671578517; NZ_JNKW01000011.1
4302; Closltidium drakei strain SL1 contig 20, whole genome shotgun sequence;
692121046; NZ_JIBUO2000020.1
4303; Rhodococcus fascians A2ld2 contig10, whole genome shotgun sequence;
739287390; NZ_JMFA01000010.1
4304; Streptomyces alboviridis strain NRRL B-1579 contig18.1, whole genome
shotgun sequence; 695845602; NZ_JNWU01000018.1
4305; Streptomyces sp. JS01 contig2, whole genome shotgun sequence;
695871554; NZ_JPWW01000002.1
4306; Streptomyces albus subsp. albus strain NRRL B-16041 contig28.1, whole
genome shotgun sequence; 695870063; NZ_JNWW01000028.1
4307; Streptomyces fimosus subsp. fimosus strain NRRL B-16073 contig7.1,
whole genome shotgun sequence; 696493030; NZ_JNWX01000007.1
4308; Streptomyces peucetius strain NRRL WC-3868 contig49.1, whole genome
shotgun sequence; 665671804; NZ_JOCK01000052.1
4309; Blautia producta strain ER3 contig 8, whole genome shotgun sequence;
696661199; NZ_JPJF01000008.1
4310; Streptomyces albus subsp. albus strain NRRL B-1811 contig32.1, whole
genome shotgun sequence; 665618015; NZ_JODR01000032.1
4311; Streptomyces lydicus strain NRRL ISP-5461 contig41.1, whole genome
shotgun sequence; 702808005; NZ_JNZA01000041.1
4312; Streptomyces iakyrus strain NRRL ISP-5482 contig6.1, whole genome
shotgun sequence; 702914619; NZ_JNXI01000006.1
4313; Kibdelosporangium afidum subsp. largum strain NRRL B-24462
contig91.4, whole genome shotgun sequence; 703243970; NZ_JNYM01001429.1
4314; Streptomyces galbus strain KCCM 41354 contig00021, whole genome
shotgun sequence; 716912366; NZ_JRHJ01000016.1
4315; Bacillus aryabhattai strain GZO3 contigl_scaffold 1, whole genome shotgun
sequence; 723602665; NZ_JPIE01000001.1
4316; Bacillus cereus R309803 chromosome, whole genome shotgun sequence;
238801472; NZ_CM000720.1
4317; Bacillus cereus AH603 chromosome, whole genome shotgun sequence;
238801489; NZ_CM000737.1
4318; Sphingomonas sp. 37zxx contig3_scaffold2, whole genome shotgun
sequence; 728813405; NZ_JR0H01000003.1
4319; Lachnospira multipara MC2003 T520DRAFT_scaffold00007.7_C, whole
genome shotgun sequence; 653225243; NZ_JHWY01000011.1
4320; Bacillus sp. 72 T409DRAFT_scf7180000000077_quiver.15S, whole
genome shotgun sequence; 736160933; NZ_JQMI01000015.1
4321; Bacillus simplex BA2H3 scaffold2, whole genome shotgun sequence;
736214556; NZ_KN360955.1
4322; Bacillus manliponensis strain JCM 15802 contig4, whole genome shotgun
sequence; 736629899; NZ_JOTN01000004.1
4323; Bacillus vietnamensis strain HD-02, whole genome shotgun sequence;
736762362; NZ_CCDN010000009.1
4324; Erythrobacter longus strain DSM 6997 contig9, whole genome shotgun
sequence; 736965849; NZ_JMIW01000009.1
4325; Calothfix sp. 336/3, complete genome; 821032128; NZ_CP011382.1
4326; Desulfobacter vibfioformis DSM 8776 Q366DRAFT_scaffold00036.35_C,
whole genome shotgun sequence; 737257311; NZ_JQKJ01000036.1
4327; Actinokineospora spheciospongiae strain EG49 contig1268_1, whole
genome shotgun sequence; 737301464; NZ_AYXG01000139.1
4328; Bacillus firmus DS1 scaffold33, whole genome shotgun sequence;
737350949; NZ_APVL01000034.1
4329; Bacillus hemicellulosilyticus JCM 9152, whole genome shotgun sequence;
737360192; NZ_BAUU01000008.1
4330; Edaphobacter aggregans DSM 19364 Q363DRAFT_scaffold00032.32_C,
whole genome shotgun sequence; 737370143; NZ_JQKI01000040.1
4331; Halobacillus sp. BBL2006 cont444, whole genome shotgun sequence;
737576092; NZ_JRNX01000441.1
4332; Bacillus akibai JCM 9157, whole genome shotgun sequence; 737696658;
NZ_BAUV01000025.1
4333; Frankia sp. CeD CEDDRAFT_scaffold_22.23, whole genome shotgun
sequence; 737947180; NZ_JPGU01000023.1
4334; Fusobacterium necrophorum BI, IR-2 contig0075, whole genome shotgun
sequence; 737951550; NZ_JAAG01000075.1
4335; [Leptolyngbya] sp. JSC-1 Osccy1DRAFT_CYJSC1 DRAF_scaffold00069.1,
whole genome shotgun sequence; 738050739; NZ_KL662191.1
4336; Lysobacter darjeonensis GH1-9 contig23, whole genome shotgun sequence;
738180952; NZ_AVPU01000014.1
4337; Mastigocoleus testarum BC008 Contig-2, whole genome shotgun sequence;
959926096; NZ_LMTZ01000085.1
4338; Myxosarcina sp. GI1 contig 5, whole genome shotgun sequence;
738529722; NZ_JRFE01000006.1
4339; Paenibacillus sp. FSL H7-689 Contig015, whole genome shotgun sequence;
738716739; NZ_ASPU01000015.1
4340; Paenibacillus sp. FSL R7-269 Contig022, whole genome shotgun sequence;
738803633; NZ_ASPS01000022.1
4341; Paenibacillus sp. FSL R7-277 Contig088, whole genome shotgun sequence;
738841140; NZ_ASPX01000088.1
4342; Prevotella oryzae DSM 17970 XylorDRAFT_X0A.1, whole genome
shotgun sequence; 738999090; NZ_KK073873.1
4343; Rothia dentocariosa strain C6B contig 5, whole genome shotgun sequence;
739372122; NZ_JQHE01000003.1
4344; Ruminococcus albus 8 contig00035, whole genome shotgun sequence;
325680876; NZ_ADKM02000123.1
4345; Amycolatopsis orientalis DSM 40040 = KCTC 9412 contig 32, whole
genome shotgun sequence; 499136900; NZ_ASJB01000015.1
4346; Streptococcus salivarius strain NU10 contig_11, whole genome shotgun
sequence; 739748927; NZ_BMT01000011.1
4347; Streptomyces griseorubens strain JSD-1 contig143, whole genome shotgun
sequence; 657284919; IIMG01000143.1
4348; Streptomyces avermitilis MA-4680 = NBRC 14893, complete genome;
162960844; NC_003155.4
4349; Streptomyces avermitilis MA-4680 = NBRC 14893, complete genome;
162960844; NC_003155.4
4350; Streptomyces aurantiacus JA 4570 Seq28, whole genome shotgun sequence;
514916412; NZ_AOPZ01000028.1
4351; Streptomyces griseus subsp. griseus strain NRRL WC-3645 contig39.1,
whole genome shotgun sequence; 739830131; NZ_JOJE01000039.1
4352; Streptomyces griseus subsp. griseus strain NRRL WC-3645 contig40.1,
whole genome shotgun sequence; 739830264; NZ_JOJE01000040.1
4353; Streptomyces scabiei strain NCPPB 4086 scf 65433_365.1, whole genome
shotgun sequence; 739854483; NZ_KL997447.1
4354; Streptomyces sp. FXJ7.023 Contig10, whole genome shotgun sequence;
510871397; NZ_APIV01000010.1
4355; Streptomyces sp. NRRL F-5053 contig1.1, whole genome shotgun
sequence; 664356765; NZ_JOHT01000001.1
4356; Streptomyces viridochromogenes Tue57 Seq127, whole genome shotgun
sequence; 443625867; NZ_AMLP01000127.1
4357; Streptomyces sp. Tu 6176 scaffold00003, whole genome shotgun sequence;
740044478; NZ_KK106990.1
4358; Streptomyces toyocaensis strain NRRL 15009 contig00064, whole genome
shotgun sequence; 740092143; NZ_JFCB01000064.1
4359; Streptomyces sp. PRh5 contig001, whole genome shotgun sequence;
740097110; NZ_JABQ01000001.1
4360; Tolypothrix bouteillei VB521301 scaffold 1, whole genome shotgun
sequence; 910242069; NZ_JHEG02000048.1
4361; Thioclava indica strain DT23-4 contig29, whole genome shotgun sequence;
740292158; NZ_AUNB01000028.1
4362; Streptomyces albulus strain NK660, complete genome; 754221033;
NZ_CP007574.1
4363; Paenibacillus sp. FSL H7-0357, complete genome; 749299172;
NZ_CP009241.1
4364; Paenibacillus stellifer strain DSM 14472, complete genome; 753871514;
NZ_CP009286.1
4365; Brevundimonas nasdar strain TPW30 Contig 13, whole genome shotgun
sequence; 746187665; NZ_MSY01000013.1
4366; Paenibacillus polymyxa strain DSM 365 Contig001, whole genome shotgun
sequence; 746220937; NZ_JMIQ01000001.1
4367; Paenibacillus sp. IHB B 3415 contig 069, whole genome shotgun sequence;
746258261; NZ_JUB01000069.1
4368; Streptomyces sp. 769, complete genome; 749181963; NZ_CP003987.1
4369; Hassallia byssoidea VB512170 scaffold 0, whole genome shotgun
sequence; 748181452; NZ_JTCM01000043.1
4370; Hassallia byssoidea VB512170 scaffold 0, whole genome shotgun
sequence; 748181452; NZ_JTCM01000043.1
4371; Jeotgalibacillus malaysiensis strain D5 chromosome, complete genome;
749182744; NZ_CP009416.1
4372; Paenibacillus sp. FSL R7-0273, complete genome; 749302091;
NZ_CP009283.1
4373; Paenibacillus jamilae strain NS115 contig 27, whole genome shotgun
sequence; 970428876; NZ_LDRX01000027.1
4374; Streptomonospora alba strain YIM 90003 contig 9, whole genome shotgun
sequence; 749673329; NZ_JR0001000009.1
4375; Actinobaculum sp. oral taxon 183 str. F0552 A_P1HMPREF0043-
1.0_Cont1.1, whole genome shotgun sequence; 541476958; AWSB01000006.1
4376; Actinobaculum sp. oral taxon 183 str. F0552 5caffold15, whole genome
shotgun sequence; 545327527; NZ_KE951412.1
4377; Actinobaculum sp. oral taxon 183 str. F0552 A_P1HMPREF0043-
1.0_Cont15.2, whole genome shotgun sequence; 541473965; AWSB01000041.1
4378; Nocardia transvalensis NBRC 15921, whole genome shotgun sequence;
485125031; NZ_BAGL01000055.1
4379; Xenococcus sp. PCC 7305 scaffold 00124, whole genome shotgun
sequence; 443325429; NZ_ALVZ01000124.1
4380; Mesorhizobium sp. ORS3324, whole genome shotgun sequence;
751265275; NZ_CCMY01000220.1
4381; Mesorhizobium plurifarium, whole genome shotgun sequence; 751292755;
NZ_CCNE01000004.1
4382; Mesorhizobium sp. SOD10, whole genome shotgun sequence; 751285871;
NZ_CCNA01000001.1
4383; Tolypothrix campylonemoides VB511288 scaffold 0, whole genome
shotgun sequence; 751565075; NZ_JXCB01000004.1
4384; Jeotgalibacillus campisalis strain SF-57 contig00001, whole genome
shotgun sequence; 751586078; NZ__ARR01000001.1
4385; Jeotgalibacillus soli strain P9 contig00009, whole genome shotgun
sequence; 751619763; NZ_JXRP01000009.1
4386; Cylindrospennum stagnale PCC 7417, complete genome; 434402184;
NC_019757.1
4387; Bacillus sp. 1NLA3E, complete genome; 488570484; NC_021171.1
4388; Tistrella mobilis KA081020-065, complete genome; 389875858;
NC_017956.1
4389; Stackebrandtia nassauensis DSM 44728, complete genome; 291297538;
NC_013947.1
4390; Magnetospirillum gryphiswaldense MSR-1, WORKING DRAFT
SEQUENCE, 373 unordered pieces; 144897097; CU459003.1
4391; Clostddium beijerinckii strain NCIMB 14988 genome; 754484184;
NZ_CP010086.1
4392; Frankia alni str. ACN14A chromosome, complete sequence; 111219505;
NC_008278.1
4393; Streptomyces sp. NBRC 110027, whole genome shotgun sequence;
754788309; NZ_BBN001000002.1
4394; Streptomyces sp. NBRC 110027, whole genome shotgun sequence;
754796661; NZ_BBN001000008.1
4395; Paenibacillus sp. FSL R7-0331, complete genome; 754821094;
NZ_CP009284.1
4396; Kibdelosporangium sp. MJ126-NF4, whole genome shotgun sequence;
754819815; NZ_CDME01000002.1
4397; Paenibacillus camerounensis strain G4, whole genome shotgun sequence;
754841195; NZ_CCDG010000069.1
4398; Paenibacillus borealis strain DSM 13188, complete genome; 754859657;
NZ_CP009285.1
4399, Streptacidiphilus neutrinimicus strain NBRC 100921, whole genome
shotgun sequence; 755016073; NZ_BBP001000030.1
4400, Streptacidiphilus melanogenes strain NBRC 103184, whole genome
shotgun sequence; 755032408; NZ_BBPP01000024.1
4401, Streptacidiphilus anmyonensis strain NBRC 103185, whole genome
shotgun sequence; 755077919; NZ_BBPQ01000048.1
4402, Streptacidiphilus jiangxiensis strain NBRC 100920, whole genome shotgun
sequence; 755108320; NZ_BBPN01000056.1
4403; Mesorhizobium sp. 0RS3359, whole genome shotgun sequence;
756828038; NZ_CCNC01000143.1
4404; Aneurinibacillus migulanus strain Nagano El contig 36, whole genome
shotgun sequence; 928874573; NZ_LIXL01000208.1
4405; Bifidobacterium reuteri DSM 23975 Contig04, whole genome shotgun
sequence; 672991374; JGZKO1000004.1
4406; Streptomyces luteus strain TRM 45540 Scaffoldl, whole genome shotgun
sequence; 759659849; NZ_KN039946.1
4407; Streptomyces nodosus strain ATCC 14899 genome; 759739811;
NZ_CP009313.1
4408; Streptomyces fradiae strain ATCC 19609 contig0008, whole genome
shotgun sequence; 759752221; NZ_JNAD01000008.1
4409; Streptomyces glaucescens strain GLA.0, complete genome; 759802587;
NZ_CP009438.1
4410; Nonomumea candida strain NRRL B-24552 contig8 1, whole genome
shotgun sequence; 759934284; NZ_JOAG01000009.1
4411; Nonomumea candida strain NRRL B-24552 contig28.1, whole genome
shotgun sequence; 759944490; NZ_JOAG01000030.1
4412; Nonomumea candida strain NRRL B-24552 contig42.1, whole genome
shotgun sequence; 759948103; NZ_JOAG01000045.1
4413; Streptomyces fulvissimus DSM 40593, complete genome; 488607535;
NC_021177.1
4414; Microcystis aeruginosa PCC 9807, whole genome shotgun sequence;
425454132; NZ_HE973326.1
4415; Streptomyces natalensis ATCC 27448 Scaffold 46, whole genome shotgun
sequence; 764442321; NZ_JRKI01000041.1
4416; Streptomyces iranensis genome assembly Siranensis, scaffold SCAF00002;
765016627; NZ_LK022849.1
4417; Risungbinella massiliensis strain GD1, whole genome shotgun sequence;
765315585; NZ_LN812103.1
4418; Paenibacillus tenae strain NRRL B-30644 contig00007, whole genome
shotgun sequence; 765319397; NZ_JTHP01000007.1
4419; Streptococcus suis strain L58I, whole genome shotgun sequence;
766595491; NZ_CEHM01000004.1
4420; Bacillus mycoides strain 11kri323 LG56_082, whole genome shotgun
sequence; 765533368; NZ_JYCJ01000082.1
4421; Paenibacillus polymyxa strain NRRL B-30509 contig00003, whole genome
shotgun sequence; 766607514; NZ_JTH001000003.1
4422; Frankia sp. CpIl-P FF86_1013, whole genome shotgun sequence;
946950294; NZ_LEX01000013.1
4423; Streptococcus suis strain B28P, whole genome shotgun sequence;
769231516; NZ_CDTB01000010.1
4424; Lachnospiraceae bacterium NK4A144 G619DRAFT_scaffold00002.2_C,
whole genome shotgun sequence; 652826657; NZ_AUJT01000002.1
4425; Lechevalieria aerocolonigenes strain NRRL B-16140 contig11.3, whole
genome shotgun sequence; 772744565; NZ_JYJG01000059.1
4426; Streptomyces sp. NRRL F-4428 contig40.2, whole genome shotgun
sequence; 772774737; NZ_JYJI01000131.1
4427; Streptomyces sp. FxanaA7 F611DRAFT_scaffold00041.41_C, whole
genome shotgun sequence; 780340655; NZ_LACL01000054.1
4428; Streptomyces rubellomurinus strain ATCC 31215 contig-63, whole genome
shotgun sequence; 783211546; NZ_JZKH01000064.1
4429; Streptomyces rubellomurinus subsp. indigoferus strain ATCC 31304 contig-
55, whole genome shotgun sequence; 783374270; NZ_JZKG01000056.1
4430; Elstera litoralis strain Dia-1 c21, whole genome shotgun sequence;
788026242; NZ_LAJY01000021.1
4431; Streptomyces sp. NRRL B-1568 contig-76, whole genome shotgun
sequence; 799161588; NZ_JZWZ01000076.1
4432; Sphingomonas sp. SRS2 contig40, whole genome shotgun sequence;
806905234; NZ_LARW01000040.1
4433; Paenibacillus wulumuqiensis strain Y24 Scaffold4, whole genome shotgun
sequence; 808051893; NZ_KQ040793.1
4434; Paenibacillus daici strain H9 Scaffold3, whole genome shotgun sequence;
808064534; NZ_KQ040798.1
4435; Spirosoma radiotolerans strain DG5A, complete genome; 817524426;
NZ_CP010429.1
4436; Allosalinactinospora lopnorensis strain CA15-2 contig00044, whole genome
shotgun sequence; 815863894; NZ_LAJC01000044.1
4437; Allosalinactinospora lopnorensis strain CA15-2 contig00053, whole genome
shotgun sequence; 815864238; NZ_LAJC01000053.1
4438; Bacillus sp. SA1-12 scf7180000003378, whole genome shotgun sequence;
817541164; NZ_LATZ01000026.1
4439; Altererythrobacter atlanticus strain 26DY36, complete genome; 927872504;
NZ_CP011452.2
4440; Streptomyces lydicus A02, complete genome; 822214995; NZ_CP007699.1
4441; Streptomyces lydicus A02, complete genome; 822214995; NZ_CP007699.1
4442; Bacillus cereus strain B4147 NODES, whole genome shotgun sequence;
822530609; NZ_LCYN01000004.1
4443; Erythrobacter luteus strain KA37 contig 1, whole genome shotgun sequence;
822631216; NZ_LBHB01000001.1
4444; Erythrobacter marinus strain HWDM-33 contig3, whole genome shotgun
sequence; 823659049; NZ_LBHU01000003.1
4445; Streptomyces sp. KE1 Contig 11, whole genome shotgun sequence;
825353621; NZ_LAYX01000011.1
4446; Sphingomonas sp. Y57 scaffold74, whole genome shotgun sequence;
826051019; NZ_LDES01000074.1
4447; Alistipes sp. ZOR0009 L990_140, whole genome shotgun sequence;
835319962; NZ_JTLD01000119.1
4448; Bacillus aryabhattai strain T61 Scaffold', whole genome shotgun sequence;
836596561; NZ_KQ087173.1
4449; Paenibacillus sp. TCA20, whole genome shotgun sequence; 843088522;
NZ_BBIW01000001.1
4450; Bacillus circulans strain RIT379 contig11, whole genome shotgun sequence;
844809159; NZ_LDPH01000011.1
4451; Bacillus circulans strain RIT379 contig11, whole genome shotgun sequence;
844809159; NZ_LDPH01000011.1
4452; Omithinibacillus califomiensis strain DSM 16628 contig 22, whole genome
shotgun sequence; 849059098; NZ_LDUE01000022.1
4453; Bacillus pseudalcaliphilus strain DSM 8725 superll, whole genome
shotgun sequence; 849078078; NZ_LFJ001000006.1
4454; Bacillus aryabhattai strain LK25 16, whole genome shotgun sequence;
850356871; NZ_LDWN01000016.1
4455; Methanobacterium formicicum genome assembly D5M1535,
chromosome: chrI; 851114167; NZ_LN515531.1
4456; Methanobacterium arcticum strain M2 EI99DRAFT_scaffold00005.5_C,
whole genome shotgun sequence; 851140085; NZ_JQKN01000008.1
4457; Methanobacterium sp. SMA-27 DL91DRAFT unitig_0_quiver. l_C, whole
genome shotgun sequence; 851351157; NZ_JQLY01000001.1
4458; Cellulomonas sp. A375-1 contig 129, whole genome shotgun sequence;
856992287; NZ_LFKW01000127.1
4459; Streptomyces sp. HNS054 contig28, whole genome shotgun sequence;
860547590; NZ_LDZX01000028.1
4460; Bacillus cereus strain RIMV BC 126 212, whole genome shotgun sequence;
872696015; NZ_LAB001000035.1
4461; Streptomyces leeuwenhoekii strain C58 contig126, whole genome shotgun
sequence; 873282818; NZ_LFEH01000123.1
4462; Bacillus sp. 220_BSPC 1447_75439_1072255, whole genome shotgun
sequence; 880954155; NZ_JVPL01000109.1
4463; Bacillus sp. 522_BSPC 2470_72498_1083579_594_ . . . _522_, whole
genome shotgun sequence; 880997761; NZ_JVDT01000118.1
4464; Bacillus sp. 522_BSPC 2470_72498_1083579_594_ . . . _522_, whole
genome shotgun sequence; 880997761; NZ_JVDT01000118.1
4465; Streptomyces varsoviensis strain NRRL B-3589 contig2.1, whole genome
shotgun sequence; 664348063; NZ_JOFN01000002.1
4466; Scytonema tolypothrichoides VB-61278 scaffold 6, whole genome shotgun
sequence; 890002594; NZ_JXCA01000005.1
4467; Erythrobacter atlanticus strain s21-N3, complete genome; 890444402;
NZ_CP011310.1
4468; Streptococcus pseudopneumoniae strain 445 SPSE
347_91401_2272315_318_ . . . _319_, whole genome shotgun sequence;
896667361; NZ_JVGV01000030.1
4469; Kitasatospora sp. MY 5-36 Contig_703_, whole genome shotgun sequence;
902792184; NZ_LFVW01000692.1
4470; Streptomyces caatingaensis strain CMAA 1322 contig02, whole genome
shotgun sequence; 906344334; NZ_LFXA01000002.1
4471; Streptomyces caatingaensis strain CMAA 1322 contig07, whole genome
shotgun sequence; 906344339; NZ_LFXA01000007.1
4472; Streptomyces caatingaensis strain CMAA 1322 contig09, whole genome
shotgun sequence; 906344341; NZ_LFXA01000009.1
4473; Candidatus Halobonum tyrrellensis G22 contig00002, whole genome
shotgun sequence; 557371823; NZ_ASGZ01000002.1
4474; Bacillus weihenstephanensis strain JAS 83/3 Bw_JAS-83/3_contig00005,
whole genome shotgun sequence; 910095435; NZ_JNLY01000005.1
4475; Silvibacterium bohemicum strain S15 contig 3, whole genome shotgun
sequence; 910257956; NZ_LBHJ01000003.1
4476; Silvibacterium bohemicum strain S15 contig 3, whole genome shotgun
sequence; 910257956; NZ_LBHJ01000003.1
4477; Silvibacterium bohemicum strain S15 contig 30, whole genome shotgun
sequence; 910257973; NZ_LBHJ01000020.1
4478; Xanthomonas campestris pv. viticola strain LMG 965, whole genome
shotgun sequence; 704493846; NZ_CBZT010000006.1
4479; Streptomyces baamensis strain NRRL B-2842 P144_Dorol_scaffold6,
whole genome shotgun sequence; 662129456; NZ_KL573544.1
4480; Streptomyces albus subsp. albus strain NRRL B-2445 contig1.1, whole
genome shotgun sequence; 664084661; NZ_JOED01000001.1
4481; Bacillus flexus strain Riq5 contig 32, whole genome shotgun sequence;
914730676; NZ_LFQJ01000032.1
4482; Salinibacter ruber M8 chromosome, complete genome; 294505815;
NC_014032.1
4483; Streptomyces vitaminophilus DSM 41686 A3IGDRAFT_scaffold_10.11,
whole genome shotgun sequence; 483682977; NZ_KB904636.1
4484; Halomonas anticariensis FP35 = DSM 16096 strain FP35 Scaffoldl, whole
genome shotgun sequence; 514429123; NZ_KE332377.1
4485; Cohnella thermotolerans DSM 17683 G485DRAFT_scaffold00003.3,
whole genome shotgun sequence; 652794305; NZ_KE386956.1
4486; Streptomyces sp. GXT6 genomic scaffold Scaffold4, whole genome
shotgun sequence; 654975403; NZ_K1601366.1
4487; Verrucomicrobia bacterium LP2A G346DRAFT_scf7180000000012_quiver.2_C,
whole genome shotgun sequence; 640169055; NZ_JAFS01000002.1
4488; Actinomadura oligospora ATCC 43269 P696DRAFT_scaffold00008.8_C,
whole genome shotgun sequence; 651281457; NZ_JADG01000010.1
4489; Actinomadura oligospora ATCC 43269 P696DRAFT_scaffold00008.8_C,
whole genome shotgun sequence; 651281457; NZ_JADG01000010.1
4490; Rubellimicrobium mesophilum DSM 19309 scaffold23, whole genome
shotgun sequence; 739419616; NZ_KK088564.1
4491; Pseudonocardia acaciae DSM 45401 N912DRAFT_scaffold00002.2_C,
whole genome shotgun sequence; 655569633; NZ_JIA101000002.1
4492; Terasakiellapusilla DSM 6293 Q397DRAFT scaffold00039.39_C, whole
genome shotgun sequence; 655499373; NZ_JHY001000039.1
4493; Bacillus sp. MB2021 T349DRAFT_scaffold00010.10_C, whole genome
shotgun sequence; 671553628; NZ_JN1101000011.1
4494; Streptomyces olindensis strain DAUFPE 5622 103, whole genome shotgun
sequence; 739918964; NZ_JJOH01000097.1
4495; Thioclava dalianensis strain DLFJ1-1 contig2, whole genome shotgun
sequence; 740220529; NZ_JHEH01000002.1
4496; Streptomyces megasporus strain NRRL B-16372 contig19.1, whole genome
shotgun sequence; 671525382; NZ_JODL01000019.1
4497; Streptomyces achromogenes subsp. achromogenes strain NRRL B-2120
contig2.1, whole genome shotgun sequence; 664063830; NZ_JODT01000002.1
4498; Microbispora rosea subsp. nonnitritogenes strain NRRL B-2631 contig12.1,
whole genome shotgun sequence; 663732121; NZ_JNZQ01000012.1
4499; Streptomyces sp. NRRL S-920 contig36.1, whole genome shotgun
sequence; 664256887; NZ_JODF01000036.1
4500; Streptomyces flavochromogenes strain NRRL B-2684 contig8.1, whole
genome shotgun sequence; 663317502; NZ_JNZ001000008.1
4501; Streptomyces natalensis strain NRRL B-5314 P055_Dorol_scaffold13,
whole genome shotgun sequence; 662108422; NZ_KL570019.1
4502; Bacillus sp. UNC322MFChir4.1 BR72DRAFT_scaffold00004.4, whole
genome shotgun sequence; 737456981; NZ_KNO50811.1
4503; Paenibacillus wynnii strain DSM 18334 unitig 2, whole genome shotgun
sequence; 738760618; NZ_JQCR01000002.1
4504; Amycolatopsis sp. MJ11V12582 contig00007, whole genome shotgun
sequence; 739487309; NZ_JPLW01000007.1
4505; Sphingopyxis fiibergensis strain Kp5.2, complete genome; 749188513;
NZ_CP009122.1
4506; Brevundimonas nasdar strain TPW30 Contig 11, whole genome shotgun
sequence; 746187486; NZ_JWSY01000011.1
4507; Microcystis panniformis FACHB-1757, complete genome; 917764592;
NZ_CP011339.1
4508; Desulfocapsa sulfexigens DSM 10523, complete genome; 451945650;
NC_020304.1
4509; Gorillibacterium massiliense strain G5, whole genome shotgun sequence;
750677319; NZ_CBQR020000171.1
4510; Salinarimonas rosea DSM 21201 G407DRAFT_scaffold00021.21_C,
whole genome shotgun sequence; 655990125; NZ_AUBC01000024.1
4511; Streptomyces sp. NRRL S-118 P205_Doro1_scaffold34, whole genome
shotgun sequence; 664565137; NZ_KL591029.1
4512; Streptomyces glaucescens strain GLA.0, complete genome; 759802587;
NZ_CP009438.1
4513; Paenibacillus sp. FSL R5-0912, complete genome; 754884871;
NZ_CP009282.1
4514; Paenibacillus sp. FSL P4-0081, complete genome; 754777894;
NZ_CP009280.1
4515; Bacillus subtilis subsp. spizizenii RFWG1A4 contig00010, whole genome
shotgun sequence; 764657375; NZ_AJHM01000010.1
4516; Paenibacillus algorifonticola strain XJ259 5caffold20_1, whole genome
shotgun sequence; 808072221; NZ_LAQ001000025.1
4517; Mycobacterium sp. UM_Kg27 contig000002, whole genome shotgun
sequence; 809025315; NZ_JRMM01000002.1
4518; Mycobacterium sp. UM_Kg 1 contig000164, whole genome shotgun
sequence; 809073490; NZ_JRMK01000164.1
4519; Streptomyces avicenniae strain NRRL B-24776 contig3.1, whole genome
shotgun sequence; 919531973; NZ_JOEK01000003.1
4520; Paenibacillus peoriae strain HS311, complete genome; 922052336;
NZ_CP011512.1
4521; Paenibacillus sp. FJAT-27812 scaffold 0, whole genome shotgun sequence;
922780240; NZ_LIGH01000001.1
4522; Hapalosiphon sp. MRB220 contig 91, whole genome shotgun sequence;
923076229; NZ_LIRN01000111.1
4523; Bacillus sp. FJAT-21352 Scaffold 1, whole genome shotgun sequence;
924654439; NZ_LIUS01000003.1
4524; Bacillus gobiensis strain FJAT-4402 chromosome; 926268043;
NZ_CP012600.1
4525; Streptomyces sp. NRRL B-1140 P439contig15.1, whole genome shotgun
sequence; 926344107; NZ_LGEA01000058.1
4526; Streptomyces sp. NRRL B-1140 P439contig32.1, whole genome shotgun
sequence; 926344331; NZ_LGEA01000105.1
4527; Streptomyces sp. NRRL F-5755 P309contig48.1, whole genome shotgun
sequence; 926371517; NZ_LGCW01000271.1
4528; Streptomyces sp. NRRL F-5755 P309contig50.1, whole genome shotgun
sequence; 926371520; NZ_LGCW01000274.1
4529; Saccharothrix sp. NRRL B-16348 P442contig71.1, whole genome shotgun
sequence; 926395199; NZ_LGED01000246.1
4530; Streptomyces sp. WM6378 P402contig63.1, whole genome shotgun
sequence; 926403453; NZ_LGDD01000321.1
4531; Streptomyces sp. WM6378 P402contig63.1, whole genome shotgun
sequence; 926403453; NZ_LGDD01000321.1
4532; Nocardia sp. NRRL S-836 P437contig3.1b, whole genome shotgun
sequence; 926412094; NZ_LGDY01000103.1
4533; Nocardia sp. NRRL S-836 P437contig39.1, whole genome shotgun
sequence; 926412104; NZ_LGDY01000113.1
4534; Paenibacillus sp. A59 contig 353, whole genome shotgun sequence;
927084730; NZ_LITU01000050.1
4535; Paenibacillus sp. A59 contig_416, whole genome shotgun sequence;
927084736; NZ_LITU01000056.1
4536; Streptomyces sp. NRRL S-444 contig322.4, whole genome shotgun
sequence; 797049078; JZWX01001028.1
4537; Streptomyces chattanoogensis strain NRRL ISP-5002 ISP5002contig8.1,
whole genome shotgun sequence; 928897585; NZ_LGKG01000196.1
4538; Streptomyces chattanoogensis strain NRRL ISP-5002 ISP5002contig9.1,
whole genome shotgun sequence; 928897596; NZ_LGKG01000207.1
4539; Bacillus sp. FJAT-28004 scaffold 2, whole genome shotgun sequence;
929005248; NZ_LGHP01000003.1
4540; Actinobacteria bacterium OK074 ctg60, whole genome shotgun sequence;
930473294; NZ_LJCV01000275.1
4541; Actinobacteria bacterium OK006 ctg112, whole genome shotgun sequence;
930490730; NZ_UCU01000014.1
4542; Actinobacteria bacterium OK006 ctg96, whole genome shotgun sequence;
930491003; NZ_LJCU01000287.1
4543; Kibdelosporangium phytohabitans strain KLBMP1111, complete genome;
931609467; NZ_CP012752.1
4544; Paenibacillus sp. GD6, whole genome shotgun sequence; 939708098;
NZ_LN831198.1
4545; Paenibacillus sp. GD6, whole genome shotgun sequence; 939708105;
NZ_LN831205 .1
4546; Alicyclobacillus ferrooxydans strain TC-34 contig 22, whole genome
shotgun sequence; 940346731; NZ_LJC001000107.1
4547; Streptomyces pactum strain ACT12 scaffold', whole genome shotgun
sequence; 943388237; NZ_LIQD01000001.1
4548; Streptomyces flocculus strain NRRL B-2465 B2465_contig_205, whole
genome shotgun sequence; 943674269; NZ_LIQ001000205.1
4549; Streptomyces aurantiacus strain NRRL ISP-5412 ISP-5412_contig_138,
whole genome shotgun sequence; 943881150; NZ_LIPP01000138.1
4550; Streptomyces graminilatus strain NRRL B-59124 B59124_contig_7, whole
genome shotgun sequence; 943897669; NZ_LIQQ01000007.1
4551; Streptomyces alboniger strain NRRL B-1832 B-1832_contig_37, whole
genome shotgun sequence; 943898694; NZ_LIQN01000037.1
4552; Streptomyces alboniger strain NRRL B-1832 B-1832_contig_384, whole
genome shotgun sequence; 943899498; NZ_LIQN01000384.1
4553; Streptomyces kanamyceticus strain NRRL B-2535 B-2535_contig_122,
whole genome shotgun sequence; 943922224; NZ_LIQU01000122.1
4554; Streptomyces kanamyceticus strain NRRL B-2535 B-2535_contig_247,
whole genome shotgun sequence; 943922567; NZ_LIQU01000247.1
4555; Streptomyces luridiscabiei strain NRRL B-24455 B24455 contig_315,
whole genome shotgun sequence; 943927948; NZ_LIQV01000315.1
4556; Streptomyces atriruber strain NRRL B-24165 contig 124, whole genome
shotgun sequence; 943949281; NZ_LIPN01000124.1
4557; Streptomyces hirsutus strain NRRL B-2713 B2713_contig_57, whole
genome shotgun sequence; 944005810; NZ_LIQT01000057.1
4558; Streptomyces aureus strain NRRL B-2808 contig 171, whole genome
shotgun sequence; 944012845; NZ_LIPQ01000171.1
4559; Streptomyces prasinus strain NRRL B-12521 B1252 l_contig_230, whole
genome shotgun sequence; 944020089; NZ_LIPRO1000230.1
4560; Streptomyces phaeochromogenes strain NRRL B-1248 B-
1248_contig_126, whole genome shotgun sequence; 944029528;
NZ_LIQZ01000126.1
4561; Streptomyces prasinus strain NRRL B-2712 B2712_contig_323, whole
genome shotgun sequence; 944410649; NZ_LIRH01000323.1
4562; Streptomyces prasinopilosus strain NRRL B-2711 B2711_contig_370,
whole genome shotgun sequence; 944415035; NZ_LIRG01000370.1
4563; Streptomyces torulosus strain NRRL B-3889 B-3889_contig_18, whole
genome shotgun sequence; 944495433; NZ_LIRK01000018.1
4564; Frankia alni str. ACN14A chromosome, complete sequence; 111219505;
NC_008278.1
4565; Paenibacillus sp. Leaf72 contig 6, whole genome shotgun sequence;
947378267; NZ_LMLV01000032.1
4566; Sanguibacter sp. Leaf3 contig 2, whole genome shotgun sequence;
947472882; NZ_LMRH01000002.1
4567; Aeromicrobium sp. Root344 contig 1, whole genome shotgun sequence;
947552260; NZ_LMDH01000001.1
4568; Sphingopyxis sp. Root1497 contig 3, whole genome shotgun sequence;
947689975; NZ_LMGF01000003.1
4569; Sphingopyxis sp. Root1497 contig 3, whole genome shotgun sequence;
947689975; NZ_LMGF01000003.1
4570; Sphingomonas sp. Root1294 contig 7, whole genome shotgun sequence;
947890193; NZ_LMEJ01000014.1
4571; Sphingomonas sp. Root720 contig 7, whole genome shotgun sequence;
947704642; NZ_LMID01000015.1
4572; Sphingomonas sp. Root720 contig 8, whole genome shotgun sequence;
947704650; NZ_LMID01000016.1
4573; Sphingomonas sp. Root710 contig_l, whole genome shotgun sequence;
947721816; NZ_LMIB01000001.1
4574; Mesorhizobium sp. Root172 contig_2, whole genome shotgun sequence;
947919015; NZ_LMHP01000012.1
4575; Mesorhizobium sp. Root102 contig 3, whole genome shotgun sequence;
947937119; NZ_LMCP01000023.1
4576; Paenibacillus sp. Soi1750 contig_l, whole genome shotgun sequence;
947966412; NZ_LMSD01000001.1
4577; Paenibacillus sp. Soi1750 contig 1, whole genome shotgun sequence;
947966412; NZ_LMSD01000001.1
4578; Paenibacillus sp. Soi1522 contig 3, whole genome shotgun sequence;
947983982; NZ_LMRV01000044.1
4579; Paenibacillus sp. Root52 contig 3, whole genome shotgun sequence;
948045460; NZ_LMF001000023.1
4580; Bacillus sp. Soi1768D1 contig 5, whole genome shotgun sequence;
950170460; NZ_LMTA01000046.1
4581; Paenibacillus sp. Soi1724D2 contig 11, whole genome shotgun sequence;
946400391; LMRY01000003.1
4582; Paenibacillus sp. Root444D2 contig 4, whole genome shotgun sequence;
950271971; NZ_LME001000034.1
4583; Paenibacillus sp. Soi1766 contig 32, whole genome shotgun sequence;
950280827; NZ_LMSJ01000026.1
4584; Paenibacillus sp. Soi1766 contig 32, whole genome shotgun sequence;
950280827; NZ_LMSJ01000026.1
4585; Streptomyces sp. Root1310 contig 5, whole genome shotgun sequence;
951121600; NZ_LMEQ01000031.1
4586; Bacillus mumlis strain DSM 16288 5caffold4, whole genome shotgun
sequence; 951610263; NZ_LMBV01000004.1
4587; Streptomyces sp. MBT76 scaffold 4, whole genome shotgun sequence;
953813790; NZ_LNBE01000004.1
4588; Gorillibacterium sp. 5N4, whole genome shotgun sequence; 960412751;
NZ_LN881722.1
4589; Thalassobius activus strain CECT 5114, whole genome shotgun sequence;
960424655; NZ_CYUE01000025.1
4590; Microbacterium testaceum strain N5283 contig 37, whole genome shotgun
sequence; 969836538; NZ_LDRU01000037.1
4591; Microbacterium testaceum strain NS206 contig 27, whole genome shotgun
sequence; 969912012; NZ_LDRS01000027.1
4592; Microbacterium testaceum strain NS183 contig 65, whole genome shotgun
sequence; 969919061; NZ_LDRR01000065.1
4593; Sphingopyxis sp. H050 H050_contig000006, whole genome shotgun
sequence; 970555001; NZ_LNRZ01000006.1

What is claimed is:

1. A method for production and optional screening of one or more lasso peptides (LPs) or one or more lasso peptide analogs or their combination using a cell-free biosynthesis (CFB) reaction mixture, comprising the steps:
(i) combining and contacting one or more lasso precursor peptides (LPP), one or more lasso core peptide (LCP), or their combination, with a lasso cyclase (LCase) enzyme, and optionally with a lasso peptidase (LPase) enzyme when the one or more LPP is present, in a CFB reaction mixture,
(ii) synthesizing the one or more lasso peptides or LP analogs in the CFB reaction mixture, and
(iii) optionally screening the one or more lasso peptides or LP analogs for one or more desired properties or activities by (1) screening the CFB reaction mixture, or (2) screening the partially purified or substantially purified lasso peptide or LP analog.

2. The method according to claim 1, further comprising:
(i) obtaining at least one of the LPP, the LCP, the LPase or the LCase by chemical synthesis or by biological synthesis, optionally
(ii) where the biological synthesis comprises transcription and/or translation of a gene or oligonucleotide encoding the LCP, a gene or oligonucleotide encoding the LPP, a gene or oligonucleotide encoding the LPAse, or a gene or oligonucleotide encoding the LCase, and
optionally
(iii) where the transcription and/or translation of these genes or oligonucleotides occurs in the CFB reaction mixture.

3. The method according to claim 2, further comprising:
(i) designing the LP gene or oligonucleotide, the LPP gene or oligonucleotide, the LPase gene or oligonucleotide, or the LCase gene or oligonucleotide for transcription and/or translation in the CFB reaction mixture, and optionally
(ii) where the designing uses genetic sequences for the lasso precursor peptide gene, the lasso core peptide gene, the lasso peptidase gene, and/or the lasso cyclase gene, and optionally
(iii) where the genetic sequences are identified using a genome-mining algorithm, and optionally where the genome-mining algorithm is anti-SMASH, BAGEL3, or RODEO.

4. The method according to any of the preceding claims wherein the combining and contacting comprises a minimal set of lasso peptide biosynthesis components in the CFB reaction mixture, where the minimal set of lasso peptide biosynthesis components comprises the one or more lasso precursor peptides (A), one lasso peptidase (B), and one lasso cyclase (C), each of which may be independently generated by the biological and/or chemical synthesis methods, or the minimal set optionally further comprises the one or more lasso core peptide and one lasso cyclase, each of which may be independently generated by the biological and/or the chemical synthesis methods.

5. The method according to anyone of the preceding claims wherein the CFB reaction mixture contains a minimal set of lasso peptide biosynthesis components and comprises one or more of:
(i) a substantially isolated lasso precursor peptide or lasso precursor peptide fusion, a substantially isolated lasso cyclase enzyme or fusion thereof, and a substantially isolated lasso peptidase enzyme or fusion thereof, or
(ii) oligonucleotides (linear or circular constructs of DNA or RNA) that encode for a lasso precursor peptide or a fusion thereof, a substantially isolated lasso cyclase enzyme or fusion thereof, and a substantially isolated lasso peptidase enzyme or fusion thereof, or
(iii) a substantially isolated precursor peptide or fusion thereof, an oligonucleotide that encodes for a lasso cyclase or fusion thereof, and an oligonucleotide that encodes for a lasso peptidase or fusion thereof, or
(iv) an oligonucleotide that encodes for a precursor peptide, an oligonucleotide that encodes for a lasso cyclase or fusion thereof, and an oligonucleotide that encodes for a lasso peptidase, or fusion thereof, or
(v) a substantially isolated lasso core peptide or fusion thereof and a substantially isolated lasso cyclase or fusion thereof, or
(vi) an oligonucleotide that encodes for a lasso core peptide and a substantially isolated lasso cyclase or fusion thereof, or
(vii) an oligonucleotide that encodes for a lasso core peptide and an oligonucleotide that encodes for a lasso cyclase or fusion thereof.

6. The method according to any one of the preceding claims wherein the lasso precursor (A) is a peptide or polypeptide produced chemically or biologically, with a sequence corresponding to the even number of SEQ ID Nos: 1-2630 or a sequence with sequence identity greater than 30% of the even number of SEQ ID Nos: 1-2630, or a protein or peptide fusion or portion thereof.

7. The method according to any one of the preceding claims wherein the lasso peptidase (B) is an enzyme produced chemically or biologically, with a sequence corresponding to peptide Nos: 1316-2336 or a natural sequence with sequence identity greater than 30% of peptide Nos: 1316-2336.

8. The method according to any one of the preceding claims wherein the lasso cyclase (C) is an enzyme produced chemically or biologically with a sequence corresponding to peptide Nos: 2337-3761 or a natural sequence with sequence identity greater than 30% of peptide Nos: 2337-3761.

9. A method according to any one of the preceding claims wherein the CFB reaction mixture further comprises one or more RiPP recognition elements (RREs) or the genes encoding such RREs.

10. The method according to any one of the preceding claims wherein the RiPP recognition elements (RREs) are proteins produced chemically or biologically with a sequence corresponding to peptide Nos: 3762-4593 or a natural sequence with sequence identity greater than 30% of peptide Nos: 3762-4593, or a protein or peptide fusion or portion thereof.

11. A method according to any one of the preceding claims wherein the CFB reaction mixture contains a lasso peptidase or a lasso cyclase that is fused at the N- or C-terminus with one or more RiPP recognition elements (RREs).

12. The method according to any one of the preceding claims wherein the one or more lasso peptide or the one or more lasso peptide analog or their combination is produced.

13. The method according to any one of the preceding claims wherein the one or more lasso peptides or the one or more lasso peptide analogs or their combination is produced and screened.

14. The method according to any one of the preceding claims wherein the one or more lasso core peptide or lasso peptide or lasso peptide analogs, containing no fusion partners, comprises at least eleven amino acid residues and a maximum of about fifty amino acid residues.

15. The method according to any one of the preceding claims wherein the CFB reaction mixture (or system) comprises a whole cell extract, a cytoplasmic extract, a nuclear extract, or any combination thereof, wherein each are independently derived from a prokaryotic or a eukaryotic cell.

16. The method according to anyone of the preceding claims wherein the CFB reaction mixture comprises substantially isolated individual transcription and/or translation components derived from a prokaryotic or a eukaryotic cell.

17. The method according to any one of the preceding claims wherein the CFB reaction mixture further comprises one or more lasso peptide modifying enzymes or genes that encode the lasso peptide modifying enzymes, and optionally wherein the one or more lasso peptide modifying enzymes is independently selected from the group consisting of N-methyltransferases, O-methyltransferases, biotin ligases, glycosyltransferases, esterases, acylases, acyltransferases, aminotransferases, amidases, hydroxylases, dehydrogenases, halogenases, kinases, RiPP heterocyclases, RiPP cyclodehydratases, and prenyltransferases.

18. The method according to any one of the preceding claims wherein the CFB reaction mixture comprises a buffered solution comprising salts, trace metals, ATP and co-factors required for activity of one or more of the LPase, the LCase, an enzyme required for the translation, an enzyme required for the transcription, or a lasso peptide modifying enzyme.

19. The method according to any one of the preceding claims wherein the CFB reaction mixture comprises the substantially isolated lasso precursor peptides or lasso core peptide, or fusions thereof, combined and contacted with the substantially isolated enzymes that include a lasso cyclase, and optionally a lasso peptidase, or fusions thereof, in a buffered solution containing salts, trace metals, ATP, and co-factors required for enzymatic activity

20. The method according to any one of the preceding claims wherein the CFB system is used to facilitate the discovery of new lasso peptides from Nature, further comprising the steps:
(i) analyzing bacterial genome sequence data and predict the sequence of lasso peptide gene clusters and associated genes, optionally using the genome-mining algorithm, optionally where the genome-mining algorithm is anti-SMASH, BAGEL3, or RODEO,
(ii) cloning or synthesizing the minimal set of lasso peptide biosynthesis genes (A-C) or oligonucleotides containing these gene sequences, and
(iii) synthesizing known or previously undiscovered natural lasso peptides using the cell-free biosynthesis methods described herein.

21. A method according to any one of the preceding claims wherein the one or more lasso peptides, the one or more lasso peptide analogs, or their combination comprises a library containing at least one lasso peptide analog in which at least one amino acid residue is changed from its natural residue.

22. A method according to any one of the preceding claims wherein the one or more lasso peptides, the one or more lasso peptide analogs, or their combination comprises a library wherein substantially all or all amino acid mutational variants of the lasso core peptide or the lasso precursor peptide, optionally where the amino acid mutational variants of the lasso core peptide or the lasso precursor peptide are obtained by biological or chemical synthesis, and optionally where the biological synthesis uses a gene library encoding substantially all or all genetic mutational variants of the lasso core peptide or the lasso precursor peptide, optionally where the gene library is rationally designed, and optionally where the mutational variants of the lasso core peptide or the lasso precursor peptide are converted to lasso peptide mutational variants, and optionally where the lasso peptide mutational variants are screened for desired properties or activities.

23. A method according to claims 21 and 22 wherein a library of lasso peptides or lasso peptide analogs is created by (1) directed evolution technologies, or (2) chemical synthesis of lasso precursor peptide or lasso core peptide variants and enzymatic conversion to lasso peptide mutational variants, or (3) display technologies, optionally wherein the display technologies are in vitro display technologies, and optionally wherein in vitro display technologies are RNA or DNA display technologies, or combination thereof, and optionally where the library of lasso peptides or lasso peptide analogs is screened for desired properties or activities.

24. A lasso peptide library, a LP analog library or a combination thereof, comprising at least two lasso peptides, at least two lasso peptide analogs, or at least one lasso peptide and one lasso peptide analog, which may be pooled together in one vessel or where each member is separated into individual vessels (e.g., wells of a plate), and wherein the library member are isolated and purified, or partially isolated and purified, or substantially isolated and purified, or optionally wherein the library members are contained in a CFB reaction mixture.

25. A library of claim 24 wherein the library is created using the methods of claims 1-5.

26. A CFB reaction mixture useful for the synthesis of lasso peptides and lasso peptide analogs comprising one or more cell extracts or cell-free reaction media that support and facilitate a biosynthetic process wherein one or more lasso peptides or lasso peptide analogs is formed by converting one or more lasso precursor peptides or one or more lasso core peptides through the action of a lasso cyclase, and optionally a lasso peptidase, and optionally wherein transcription and/or translation of oligonucleotide inputs occurs to produce the lasso cyclase, lasso peptidase, lasso precursor peptides, and/or lasso core peptides.

27. A CFB reaction mixture of claim 26 further comprising a supplemented cell extract.

28. A CFB reaction mixture of claims 26 and 27 also comprising the oligonucleotides, genes, biosynthetic gene clusters, enzymes, proteins, and final peptide products, including lasso precursor peptides, lasso core peptides, lasso peptides, or lasso peptide analogs that result from performing a CFB reaction.

29. A kit for the production of lasso peptides and/or lasso peptide analogs according to any of the preceding claims comprising a CFB reaction mixture, a cell extract or cell extracts, cell extract supplements, a lasso precursor peptide or gene or a library of such, a lasso core peptide or gene or a library of such, a lasso cyclase or gene or genes, and/or a lasso peptidase or gene, along with information about the contents and instructions for producing lasso peptides or lasso peptide analogs.

30. A lasso peptidase library comprising at least two lasso peptidases, wherein the lasso peptidases are encoded by genes of a same organism or encoded by genes of different organisms.

31. The lasso peptidase library of claim 30, wherein each lasso peptidase of the at least two lasso peptidases comprises an amino acid sequence selected from peptide Nos: 1316-2336.

32. The lasso peptidase library of anyone of claims 30-31, wherein the library is produced by a cell-free biosynthesis system.

33. A lasso cyclase library comprising at least two lasso cyclases, wherein the lasso cyclases are encoded by genes of a same organism or encoded by genes of different organisms.

34. The lasso cyclase library of claim 33, wherein each lasso peptidase of the at least two lasso cyclases comprises an amino acid sequence selected from peptide Nos: 2337-3761.

35. The lasso cyclase library of any one of claims 33-34, wherein the library is produced by a cell-free biosynthesis system.

36. A cell free biosynthesis (CFB) system for producing one or more lasso peptide or lasso peptide analogs, wherein the CFB system comprises at least one component capable of producing one or more lasso precursor peptide.

37. The CFB system of claim 36, wherein the CFB system further comprises at least one component capable of producing one or more lasso peptidase.

38. The CFB system of claim 37, wherein the CFB system further comprises at least one component capable of producing one or more lasso cyclase.

39. The CFB system of any one of claims 36-38, wherein the at least one component capable of producing the one or more lasso precursor peptide comprises the one or more lasso precursor peptide.

40. The CFB system of any one of claims 36-39, wherein the one or more lasso precursor peptide is synthesized outside the CFB system.

41. The CFB system of any one of claims 36-39, wherein the one or more lasso precursor peptide is isolated from a naturally-occurring microorganism.

42. The CFB system of any one of claims 36-39, wherein the one or more lasso precursor peptide is isolated from a plurality naturally-occurring microorganisms.

43. The CFB system of claim 41 or 42, wherein the lasso precursor peptide is isolated as a cell extract of the naturally occurring microorganism.

44. The CFB system of any one of claims 36-43, wherein the at least one component capable of producing the one or more lasso precursor peptide comprises a polynucleotide encoding for the one or more lasso precursor peptide.

45. The CFB system of claim 44, wherein the polynucleotide comprises a genomic sequence of a naturally-existing microbial organism.

46. The CFB system of claim 45, wherein the polynucleotide comprises a mutated genomic sequence of a naturally-existing microbial organism.

47. The CFB system of any one of claims 44 to 46, wherein the polynucleotide comprises a plurality polynucleotides.

48. The CFB system of claim 47, wherein the plurality of polynucleotides each comprises a genomic sequence of a naturally existing microbial organism and/or a mutated genomic sequence of a naturally existing microbial organism.

49. The CFB system of claim 47, wherein at least two of the plurality of polynucleotides comprise genomic sequences or mutated genomic sequences of different naturally existing microbial organisms.

50. The CFB system of any one of claims 43 to 49 wherein the polynucleotide comprises a sequence selected from the odd numbers of SEQ ID Nos: 1-2630 or a homologous sequence thereof.

51. The CFB system of any one of claims 36-50, wherein the at least one component capable of producing the one or more lasso peptidase comprises the one or more lasso peptidase.

52. The CFB system of any one of claims 36-51, wherein the one or more lasso peptidase is synthesized outside the CFB system.

53. The CFB system of any one of claims 36-52, wherein the one or more lasso peptidase is isolated from a naturally-occurring microorganism.

54. The CFB system of claim 53, wherein the lasso peptidase is isolated as a cell extract of the naturally occurring microorganism.

55. The CFB system of any one of claims 36-54, wherein the at least one component capable of producing the one or more lasso peptidase comprises a polynucleotide encoding for the one or more lasso peptidase.

56. The CFB system of claim 55, wherein the polynucleotide encoding for the lasso peptidase comprises a genomic sequence of a naturally-existing microbial organism.

57. The CFB system of claim 56, wherein the polynucleotide encoding for the one or more lasso peptidase comprises a plurality of polynucleotide encoding for the one or more lasso peptidase.

58. The CFB system of claim 55 or 56, wherein the plurality of polynucleotides each comprises a genomic sequence of a naturally existing microbial organism.

59. The CFB system of claim 58, wherein at least two of the plurality of polynucleotides encoding the one or more lasso peptidase comprise genomic sequences of different naturally existing microbial organisms.

60. The CFB system of any one of claims 36-59, wherein the at least one component capable of producing the one or more lasso cyclase comprises the one or more lasso cyclase.

61. The CFB system of any one of claims 36-60, wherein the one or more lasso cyclase is synthesized outside the CFB system.

62. The CFB system of any one of claims 36-61, wherein the one or more lasso cyclase is isolated from a naturally-occurring microorganism.

63. The CFB system of any one of claims 36-61, wherein at least two of the one or more lasso cyclases are isolated from different naturally-occurring microorganisms.

64. The CFB system of claim 62 or 63, wherein the lasso peptidase is isolated as a cell extract of the naturally occurring microorganism.

65. The CFB system of any one of claims 36-64, wherein the at least one component capable of producing the one or more lasso cyclase comprises a polynucleotide encoding for the one or more lasso cyclase.

66. The CFB system of any one of claims 36-64, wherein the at least one component capable of producing the one or more lasso cyclase comprises a plurality of polynucleotides encoding for the one or more lasso cyclase.

67. The CFB system of claim 65 or 66, wherein the polynucleotide encoding for the lasso cyclase comprises a genomic sequence of a naturally-existing microbial organism.

68. The CFB system of claim 66 or 67, wherein at least two of the plurality of polynucleotides encoding the one or more lasso cyclase comprise genomic sequences of different naturally existing microbial organisms.

69. The CFB system of any one of claims 43 to 68, wherein the one or more lasso precursor peptide each comprises an amino acid sequence selected from the even number of SEQ ID Nos: 1-2630 or a homologous sequence having at least 30% sequence identity to the even number of SEQ ID Nos: 1-2630.

70. The CFB system of any one of claims 43 to 69, wherein the one or more lasso peptidase each comprises an amino acid sequence selected from peptide Nos: 1316-2336.

71. The CFB system of any one of claims 43 to 70, wherein the one or more lasso peptidase each comprises an amino acid sequence selected from peptide Nos: 2337-3761.

72. The CFB system of anyone of claims 43 to 71, further comprises at least one component capable of producing one or more RIPP recognition element (RRE).

73. The CFB system of claim 72, wherein the one or more RRE each comprises an amino acid sequence selected from peptide Nos: 3762-4593.

74. The CFB system of claim 72 or 73, wherein the at least one component capable of producing the one or more RRE comprises the one more RRE.

75. The CFB system of claim 72 or 74, wherein the RRE comprises at least one component capable of producing the one or more RRE comprises a polynucleotide encoding for the one or more RRE.

76. The CFB system of claim 75, wherein the polynucleotide encoding for the one or more RRE comprises a plurality of polynucleotides encoding for the one or more RRE.

77. The CFB system of claim 75 or 76, wherein the polynucleotide encoding for the one or more RRE comprises a genomic sequence or a naturally existing microorganism.

78. The CFB system of claim 76, wherein at least two of the plurality of polynucleotides encoding the one or more RREs comprise genomic sequences of different naturally existing microbial organisms.

79. The CFB system according to any one of claims 36 to 78 wherein the CFB system comprises a minimal set of lasso biosynthesis components.

80. The CFB system according to any one of claims 36-79, wherein the CFB system is capable of producing a combination of (i) lasso precursor peptide or a lasso core peptide, (ii) lasso cyclase, and (iii) lasso peptidase as listed in Table 1.

81. The CFB system according to any one of claims 36-79, wherein the CFB system is capable of producing a lasso peptide library.

82. The CFB system according to any one of claims 36-81, wherein the CFB system comprises a cell extract.

83. The CFB system according to any one of claims 36-82, wherein the CFB system comprises a supplemented cell extract.

84. The CFB system according to any one of claims 36-83, wherein the CFB system comprises a CFB reaction mixture.

85. The CFB system according to any one of claims 36-84, wherein the CFB system is capable of producing at least one lasso peptide or lasso peptide analog when incubated under a suitable condition.

86. The CFB system according to claim 85, wherein the suitable condition is a substantially anaerobic condition.

87. The CFB system according to claim 85, wherein the CFB comprises a cell extract, and the suitable condition comprises the natural growth condition of the cell where the cell extract is derived.

88. The CFB system according to any one of claims 36-87, wherein the CFB system is in the form of a kit.

89. The CFB system according to claim 88, wherein the one or more components of the CFB systems are separated into a plurality of parts forming the kit.

90. The CFB system according to claim 89, the plurality of parts forming the kit, when separated from one another, are substantially free of chemical or biochemical activity.
